(19) United States
(12) Patent Application Publication
(10) Pub. No.: US 2026/0258376 A1
(43) Pub. Date: Sep. 3, 2026

(54) COMPOSITIONS AND METHODS FOR EPIGENETIC REGULATION OF CIITA EXPRESSION

(71) Applicant: Chroma Medicine, Inc., Boston, MA (US)

(72) Inventors: Jamie Lynn Schafer, Boston, MA (US); Noorussahar Abubucker, Watertown, MA (US); Ricardo Noel Ramirez, Hyde Park, MA (US); Ari Friedland, Cambridge, MA (US); Morgan Maeder, Waban, MA (US); Vic Myer, Arlington, MA (US)

(73) Assignee: Chroma Medicine, Inc., Boston, MA (US)

(21) Appl. No.: 18/877,836

(22) PCT Filed: Jun. 23, 2023
(86) PCT No.: PCT/US2023/069033

§ 371 Date: Dec. 20, 2024

Related U.S. Application Data
(60) Provisional application No. 63/355,063, filed on Jun. 23, 2022.

(51) Int. Cl. C12N 9/10; A61K 38/00; A61P 37/06; C07K 14/47; C12N 9/22; C12N 15/11; C12N 15/88

(52) U.S. Cl. CPC C12N 9/1007; A61P 37/06; C07K 14/4703; C12N 9/226; C12N 15/111; C12N 15/88; A61K 38/00; C07K 2319/09; C07K 2319/80; C12N 2310/11; C12Y 201/01037

(57) ABSTRACT

The present disclosure relates to compositions and methods comprising epigenetic editors for epigenetic modification of CIITA, as well as nucleic acids and vectors encoding the same. Also disclosed are cells epigenetically modified by the epigenetic editors.

### CROSS-REFERENCE TO RELATED APPLICATIONS

This application claims the benefit under 35 U.S.C. § 119 (e) of U.S. Provisional Application No. 63/355,063, filed Jun. 23, 2023, entitled “COMPOSITIONS AND METHODS FOR EPIGENETIC REGULATION OF CIITA EXPRESSION,” the entire disclosure of each of which is hereby incorporated by reference in its entirety.

### REFERENCE TO AN ELECTRONIC SEQUENCE LISTING

The contents of the electronic sequence listing (C169870010WO00-SEQ-AXW.xml; Size: 2,284,448 bytes; and Date of Creation: Jun. 23, 2023) is herein incorporated by reference in its entirety.

### BACKGROUND

Adoptive cell therapy using genetically engineered immune cells has emerged as a promising approach to treat cancer, infections, autoimmune diseases, and other disorders. However, traditional genetic engineering strategies typically rely on permanent manipulation of cells at the genomic level, which is associated with certain risks, including, for example, chromosomal translocations, undesired insertions and deletions of nucleotides at the targeted site, and off-target mutations. There remains a need for efficient and safe methods of genetically engineering immune cells.

### SUMMARY

The present disclosure provides systems and compositions for epigenetic modification (“epigenetic editors” or “epigenetic editing systems” herein), and methods of using the same to generate epigenetic modification at CIITA, including in host cells and organisms.

In some aspects, the present disclosure provides a system for repressing transcription of a human CIITA gene in a human cell, optionally a human T lymphocyte or a human NK cell, comprising
a) one or more fusion proteins that collectively comprise
a DNA methyltransferase (DNMT) domain and/or a domain that recruits a DNMT, optionally wherein the DNMT domain and/or the recruiter domain comprise a DNMT3A domain and/or a DNMT3L domain, and optionally wherein the recruited DNMT is DNMT3A, and a transcriptional repressor domain,
each domain being linked to a DNA-binding domain that binds to a target region in the human CIITA gene; or
b) one or more nucleic acid molecules encoding the one or more fusion proteins.

In some aspects, the system comprises
a) a single fusion protein comprising the DNMT3A domain, the DNMT3L domain, the transcriptional repressor domain, and the DNA-binding domain, or
b) a nucleic acid molecule encoding the single fusion protein.

In some embodiments, the DNA-binding domain comprises a dead CRISPR Cas (dCas) domain, a ZFP domain, or a TALE domain. For example, the DNA-binding domain may comprise a dCas9 domain, and the system may further comprise (i) one or more guide RNAs comprising any one of SEQ ID NOs: 1034-1312), or (ii) nucleic acid molecules coding for the one or more guide RNAs.

In certain embodiments, the dCas domain comprises a dCas9 sequence, such as a sequence with at least 90% identity to SEQ ID NO: 12 or 13.

In some embodiments, the DNA-binding domain binds to a target sequence in SEQ ID NO: 1313 or 1314.

In some embodiments, the DNA-binding domain comprises a ZFP domain that targets a nucleotide sequence selected from SEQ ID NOs: 700-754.

In some embodiments, the DNMT3A domain comprises a sequence with at least 90% identity to SEQ ID NO: 574 or 575.

The DNMT3L domain may comprise, e.g., a sequence with at least 90% identity to a sequence selected from SEQ ID NOs: 578-581. In some embodiments, the DNMT3L domain comprises a sequence with at least 90% identity to a sequence selected from SEQ ID NOs: 582-603. In some embodiments, the DNMT3L domain comprises a sequence with at least 90% identity to a sequence selected from SEQ ID NOs: 601-603.

In some embodiments, the transcriptional repressor domain comprises a sequence with at least 90% identity to a sequence selected from SEQ ID NOs: 33-570. In certain embodiments, the transcriptional repressor domain is a KRAB domain derived from KOX1, ZIM3, ZFP28, or ZN627. The KRAB domain may comprise, e.g., a sequence with at least 90% identity to a sequence selected from SEQ ID NOs: 89, 116, 245, and 255. In some embodiments, the transcriptional repressor domain comprises a fusion of the N- and C-terminal regions of ZIM3 and KOX1 KRAB, and optionally comprises the amino acid sequence of SEQ ID NO: 571 or 572. In certain embodiments, the transcriptional repressor domain is derived from KAP1, MECP2, HP1a/CBX5, HP1b, CBX8, CDYL2, TOX, TOX3, TOX4, EED, EZH2, RBBP4, RCOR1, or SCML2.

In some embodiments, the system comprises
a) a fusion protein comprising the DNMT3A domain, the DNMT3L domain, the transcriptional repressor domain, and the DNA-binding domain,
optionally wherein one or both of the DNMT3A domain and the DNMT3L domain are human, and
optionally wherein the DNA-binding domain is a dead CRISPR Cas domain or a ZFP domain; or
b) a nucleic acid molecule encoding the fusion protein.

In certain embodiments, the fusion protein comprises, from N-terminus to C-terminus, the DNMT3A domain, a first peptide linker, the DNMT3L domain, a second peptide linker, the DNA-binding domain, a third peptide linker, and the transcriptional repressor domain. For example, the fusion protein may comprise, from N-terminus to C-terminus, the DNMT3A domain, the first peptide linker, the DNMT3L domain, the second peptide linker, a first nuclear localization signal (NLS), the DNA-binding domain, a second NLS, the third peptide linker, and the transcriptional repressor domain. The fusion protein may comprise, from N-terminus to C-terminus, a first NLS, the DNMT3A domain, the first peptide linker, the DNMT3L domain, the second peptide linker, the DNA-binding domain, the third peptide linker, the transcriptional repressor domain, and a second NLS. The fusion protein may comprise, from N-terminus to C-terminus, first and second NLSs, the DNMT3A domain, the first peptide linker, the DNMT3L domain, the second peptide linker, the DNA-binding domain, the third peptide linker, the transcriptional repressor domain, and third and fourth NLSs. In particular embodiments, the transcriptional repressor domain is a KRAB domain, such as a human KOX1, ZFP28, ZN627, or ZIM3 KRAB domain. In particular embodiments, one or both of the second and third peptide linkers are XTEN linkers, which may be selected from XTEN80 (e.g., SEQ ID NO: 643) and XTEN16 (e.g., SEQ ID NO: 638), e.g., wherein the second peptide linker is XTEN80, and the third peptide linker is XTEN16.

In some embodiments, the fusion protein may comprise, from N-terminus to C-terminus, a human DNMT3A domain, a first peptide linker, a human DNMT3L domain, an XTEN80 peptide linker, a first NLS, a dSpCas9 domain, a second NLS, an XTEN16 peptide linker, and a human KOX1 KRAB domain. In certain embodiments, the fusion protein comprises SEQ ID NO: 658 or a sequence at least 90% identical thereto.

In some embodiments, the fusion protein comprises, from N-terminus to C-terminus, a human DNMT3A domain, a first peptide linker, a human DNMT3L domain, an XTEN80 peptide linker, a first NLS, a ZFP domain, a second NLS, an XTEN16 linker, and a human KOX1 KRAB domain. In certain embodiments, the fusion protein comprises SEQ ID NO: 659 or a sequence at least 90% identical thereto.

In some embodiments, the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human KOX1 KRAB domain, and third and fourth NLSs. In particular embodiments, the fusion protein may comprise the amino acid sequence of SEQ ID NO: 660 or a sequence at least 90% identical thereto.

In some embodiments, the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human KOX1 KRAB domain, and third and fourth NLSs.

In some embodiments, the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human ZFP28 KRAB domain, and third and fourth NLSs. In particular embodiments, the fusion protein may comprise the amino acid sequence of SEQ ID NO: 661 or a sequence at least 90% identical thereto.

In some embodiments, the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human ZFP28 KRAB domain, and third and fourth NLSs.

In some embodiments, the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human ZN627 KRAB domain, and third and fourth NLSs. In particular embodiments, the fusion protein may comprise the amino acid sequence of SEQ ID NO: 662 or a sequence at least 90% identical thereto.

In some embodiments, the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human ZN627 KRAB domain, and third and fourth NLSs.

In some embodiments, the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human ZIM3 KRAB domain, and third and fourth NLSs. In particular embodiments, the fusion protein may comprise the amino acid sequence of SEQ ID NO: 663 or a sequence at least 90% identical thereto.

In some embodiments, the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human ZIM3 KRAB domain, and third and fourth NLSs.

In some embodiments, at least one of the NLSs in a fusion protein described herein is an SV40 NLS (e.g., SEQ ID NO: 644).

In some embodiments, the system comprises:
a) a first fusion protein comprising a first DNA-binding domain and comprising or recruiting the DNMT3A domain,
a second fusion protein comprising a second DNA-binding domain and comprising or recruiting the DNMT3L domain, and
a third fusion protein comprising a third DNA-binding domain and comprising or recruiting the transcriptional repressor domain; or
b) one or more nucleic acid molecules encoding the fusion proteins.

The present disclosure also provides a human cell comprising a system described herein, or progeny of the cell. In some embodiments, the cell is a T lymphocyte or a NK cell.

The present disclosure also provides a human cell modified (optionally ex vivo) by a system described herein, or progeny of the cell. In some embodiments, the cell is a T lymphocyte or a NK cell.

The present disclosure also provides a pharmaceutical composition comprising a system described herein and a pharmaceutically acceptable excipient. In some embodiments, the composition comprises lipid nanoparticles (LNPs) comprising the system, and/or the DNA-binding domain is a dCas domain and the LNPs further comprise one or more gRNAs.

The present disclosure also provides a pharmaceutical composition comprising human cells as described herein and a pharmaceutically acceptable excipient.

The present disclosure also provides a method of treating a patient in need thereof, comprising administering a system, human cells, or a pharmaceutical composition described herein to the patient (e.g., intravenously). In some embodiments, the patient has cancer or autoimmune disease.

The present disclosure also provides a system, human cells, or a pharmaceutical composition described herein for use in treating a patient in need thereof, e.g., in a method described herein.

The present disclosure also provides use of a system or human cells described herein in the manufacture of a medicament for treating a patient in need thereof, e.g., in a method described herein.

The present disclosure also provides articles and kits comprising the systems or human cells described herein.

Other features, objectives, and advantages of the invention are apparent in the detailed description that follows. It should be understood, however, that the detailed description, while indicating embodiments and embodiments of the invention, is given by way of illustration only, not limitation. Various changes and modifications within the scope of the invention will become apparent to those skilled in the art from the detailed description.

### BRIEF DESCRIPTION OF THE DRAWINGS

FIGS. 1A-1D are scatter plots showing the relative HLA-DR (MHC Class II) expression (y-axis) at day 6 (FIGS. 1A and 1C) and day 20 (FIGS. 1B and 1D) in cells treated with a CRISPR-off epigenetic editing system (DNMT3A-3L-dCas9-KRAB plus gRNAs) versus cells treated with no gRNAs. The distance from the gRNA target site to the CIITA transcription start site (TSS) is shown on the x-axis. An alternative TSS containing a well-annotated CpG island (CGI) is also indicated (at about 2 kb downstream of the canonical TSS).

FIG. 2 shows silencing of CIITA observed with non-limiting guide RNAs on Day 6 post-administration, separated by donor.

FIGS. 3A-3B shows the results of a CIITA gRNA screen using pairs of CIITA-targeting gRNAs (“duplex” CIITA targeting). Results are shown over time. The lefthand panel is not normalized; righthand is normalized.

FIG. 4 shows the gating strategy for a dose response experiment performed using duplex CIITA targeting gRNA pairs.

FIGS. 5-8 show the results of dose response experiments utilizing the specified gRNAs.

FIG. 9 shows a timecourse of a screen of CIITA-targeting zinc finger (ZF) domains.

FIG. 10 shows silencing by ZF constructs binding at various distances from the CIITA TSS.

FIGS. 11A-11B show the performance of indicated epigenetic editors and guides in two donors in frozen cells.

FIGS. 12-15 show silencing of CIITA using depicted CRISPR-Off variants. FIG. 12 shows the gating strategy employed. FIG. 13 (not normalized) FIG. 14 (normalized) show CIITA silencing over time.

FIGS. 15-17 show the results of transduction timing experiments. FIG. 15 compares results at day 6 for cells nucleofected at day 2 or day 3. FIG. 16 shows transduction efficiency. FIG. 17 shows the results for CAR+ and CAR− cells.

FIGS. 18A-18B show the results of RNAseq experiments, including CIITA mRNA/FACS comparisons (FIG. 18A) and a differentially expressed gene (DEG) summary (FIG. 18B). FIG. 18A shows the differential expression of CIITA and presence of cell-surface CIITA in cells treated with WTCas9, effector only, or effector+gRNA. FIG. 18B shows the number of differentially expressed genes under relevant conditions shown in the legend.

FIG. 19A-19B show exon level differences of CIITA expression between WTCas9 and CRISPR-Off. Results indicate that CRISPR-Off reduces CIITA isoform/exon expression more robustly than WTCas9.

FIGS. 20A-20C show differential expression of genes in cells epigenetically edited with RNA_232_269 compared with an effector only control (FIG. 8B) or WTCas9 (FIG. 8C).

FIGS. 21A-21C show differential expression of genes in cells epigenetically edited with RNA_236_269 compared with an effector only control (FIG. 9B) or WTCas9 (FIG. 9C).

FIGS. 22A-22C show differential expression of genes in cells epigenetically edited with RNA_268_241 compared with an effector only control (FIG. 10B) or WTCas9 (FIG. 10C).

FIGS. 23A-23C show differential expression of genes in cells epigenetically edited with RNA_265_268 compared with an effector only control (FIG. 11B) or WTCas9 (FIG. 11C).

FIGS. 24A-24C show differential expression of genes in cells epigenetically edited with RNA_268_339 compared with an effector only control (FIG. 12B) or WTCas9 (FIG. 12C).

FIGS. 25A-25C show differential expression of genes in cells epigenetically edited with RNA_269_339 compared with an effector only control (FIG. 12B) or WTCas9 (FIG. 12C).

FIG. 26A-26B shows down-regulated differentially expressed genes (vs. Effector control) that are shared between guide RNA pairs and an interaction map of some DEGs. FIG. 26B shows details of the CIITA pathway.

FIG. 27A-27B show performance of ZF constructs (as measured by % HLA-DR+), both normalized (FIG. 27A) and normalized to off-target (FIG. 27B).

### DETAILED DESCRIPTION OF THE INVENTION

The present disclosure provides epigenetic editors for repressing expression of the human CIITA gene. By altering expression of CIITA, the editors herein may be used to generate allogeneic cells (e.g., T cells, NK cells, etc.) with reduced alloreactivity. Unless otherwise stated, “CIITA” (in italic) refers herein to a human CIITA gene. A human CIITA gene sequence can be found at Ensembl Accession No. ENSG00000179583.21. The present epigenetic editors have several advantages compared to other genome engineering methods, including reversibility, decreased risk of chromosomal translocation, and durable, inheritable silencing.

In some embodiments, the region of the human CIITA gene targeted for epigenetic regulation is about 2 kb long, and is approximately +/−1 kb of the CIITA transcription start site (TSS). In certain embodiments, the region has the nucleotide sequence of SEQ ID NO: 1314 (shown below). In some embodiments, the targeted CIITA region is about 1000 bps long, and is approximately +/−500 bps of the CIITA TSS. In certain embodiments, the region targeted has the nucleotide sequence of SEQ ID NO: 1313 (shown below). TSS1 of CIITA is at #chr16: 10877202 and TSS2 (alternative TSS) of CIITA is at #chr16: 10878968 of Genome GRCh38: CM000678.2.

(SEQ ID NO: 1313)
TCCAATGTCTTGGGATGAAAATGACAGGTGGGCCACTTATGATCT
CCAGAGAAATTCAGGGCAATTTGGTGTGGGAGTAGGCATGGTAGA
GGAGAGCAGCATCTAAGAAGTCCCCAGCAGAGGCTCTCAGCTTGT
CTTGAGGCATCTGGGCGGAGGGCTATGATACTGGCCCCATCCTGC
AGAAGGTGGCAGATATTGGCAGCTGGCACCAGTGCGGTTCCATTG
TGATCATCATTTCTGAACGTCAGACTGTTGAAGGTTCCCCCAACA
GACTTTCTGTGCAACTTTCTGTCTTCACCAAATTCAGTCCACAGT
AAGGAAGTGAAATTAATTTCAGAGGTGTGGGGAGGGCTTAAGGGA
GTGTGGTAAAATTAGAGGGTGTTCAGAAACAGAAATCTGACCGCT
TGGGGCCACCTTGCAGGGAGAGTTTTTTTGATGATCCCTCACTTG
TTTCTTTGCATGTTGGCTTAGCTTGGCGGGCTCCCAACTGGTGAC
TGGTTAGTGATGAGGCTAGTGATGAGGCTGTGTGCTTCTGAGCTG
GGCATCCGAAGGCATCCTTGGGGAAGCTGAGGGCACGAGGAGGGG
CTGCCAGACTCCGGGAGCTGCTGCCTGGCTGGGATTCCTACACAA
TGCGTTGCCTGGCTCCACGCCCTGCTGGGTCCTACCTGTCAGAGC
CCCAAGGTAAAAAGGCCGGGAAAGCATCTTAATTTAGCGTGCAGT
CTCAGCTGGTCCTGCCATTCCAGATAAACAGAGAAACCATTCTGA
ATTGGGGATGGGGGTGAGGATGGGAACAGGAGTCTGTGTCCTGCT
GGGGCAGGCCATTGGAAGATGTGAAAGAGTTGTCTATTTCCTTCC
ACCGGAGGGAGACTTCAGGTCAGCCAGGTGTCTGGAGTATGAACC
ATGTATCAGCACCGAAAGGTTCTAGAAGTCAGACTTTCGGGCAGT
GTGTCACTAACTCTCAGCATGCTGGCCTGGCTCGGCCCACAGCAA
GGTCTTCTCGC
(SEQ ID NO: 1314)
ATAGGGTGTCACTATGTTGCCCAGGCTAGCCTCCAACTCCCGGCT
TCAAGCAATCCTCCTGCTTCGGCCTCCCAAAATGTTGGAATTACA
GGCACAAGCCACCTGGCCCAGCCATCTACTTTATATTCAAATAAA
ACTTTACGTCCCATTATAAAGGGAAAAAATGGCAAAAACAGGAGG
TAACCATTTAACAAGAAAGCAGAGTGATGTTAGATTATAGCAAGA
TACTGTTGACTGTAGAAGGCTCTGAGGCTAGAGAGCTGCTTTCTA
TAAAACAGAGTGATCATATATTAGAAGAGGTGTTAAAGACATGTT
CACACCAAGCTGAGACTTCCTCCTTGATACCACCAGGAGGATGGG
CAGAGACTGGAAAAGACACTAACTTTCTCCCTATGGGAGTCAGTA
TTATTTAGCATCACTTTGGCGGGTCACCCCAAACCATCTGACTAC
AAGGGTACCATATTTGGGTTAACACTCTTTTGGTATAATTTATGT
TTTAGTCCAATGTCTTGGGATGAAAATGACAGGTGGGCCACTTAT
GATCTCCAGAGAAATTCAGGGCAATTTGGTGTGGGAGTAGGCATG
GTAGAGGAGAGCAGCATCTAAGAAGTCCCCAGCAGAGGCTCTCAG
CTTGTCTTGAGGCATCTGGGCGGAGGGCTATGATACTGGCCCCAT
CCTGCAGAAGGTGGCAGATATTGGCAGCTGGCACCAGTGCGGTTC
CATTGTGATCATCATTTCTGAACGTCAGACTGTTGAAGGTTCCCC
CAACAGACTTTCTGTGCAACTTTCTGTCTTCACCAAATTCAGTCC
ACAGTAAGGAAGTGAAATTAATTTCAGAGGTGTGGGGAGGGCTTA
AGGGAGTGTGGTAAAATTAGAGGGTGTTCAGAAACAGAAATCTGA
CCGCTTGGGGCCACCTTGCAGGGAGAGTTTTTTTGATGATCCCTC
ACTTGTTTCTTTGCATGTTGGCTTAGCTTGGCGGGCTCCCAACTG
GTGACTGGTTAGTGATGAGGCTAGTGATGAGGCTGTGTGCTTCTG
AGCTGGGCATCCGAAGGCATCCTTGGGGAAGCTGAGGGCACGAGG
AGGGGCTGCCAGACTCCGGGAGCTGCTGCCTGGCTGGGATTCCTA
CACAATGCGTTGCCTGGCTCCACGCCCTGCTGGGTCCTACCTGTC
AGAGCCCCAAGGTAAAAAGGCCGGGAAAGCATCTTAATTTAGCGT
GCAGTCTCAGCTGGTCCTGCCATTCCAGATAAACAGAGAAACCAT
TCTGAATTGGGGATGGGGGTGAGGATGGGAACAGGAGTCTGTGTC
CTGCTGGGGCAGGCCATTGGAAGATGTGAAAGAGTTGTCTATTTC
CTTCCACCGGAGGGAGACTTCAGGTCAGCCAGGTGTCTGGAGTAT
GAACCATGTATCAGCACCGAAAGGTTCTAGAAGTCAGACTTTCGG
GCAGTGTGTCACTAACTCTCAGCATGCTGGCCTGGCTCGGCCCAC
AGCAAGGTCTTCTCGCCTCCCTTTGGGTAAATACTGAGGGGTGCC
TCTGCAGGACGGGACCTCTGCCAGACTCCACTCCATACCCAGAGA
AGCAGGGAAACCAAAATTGGAGTCAGCCTTGAGGTGTAGCTGTTG
AGCCCTCAGCAGCTGGGGAGAGCTGGCGGATGCTGCCCTCCCCCC
AGTTTCCTAATGGTGTTGTTTAAAAAGGGTCAGGGGACGGGGGAA
CAGATGGTGGGAAGAGCACAGTGCAGACACCTGGCACCGGCTCTG
AAGGCAGCATGGCAGCTACACCGTTGGCTGGGAAGGGTGTGCCCC
TGAAGAAGTCGTTTACATTCTCGAGTCAATTTTCCTGGAGTGTAC
AATGGACCTGTGGGAAAGCCTGTATGAAAGGGTAATGATGAGGGA
CCTAGCACAGTGTCCAATATTTTATAGGAACTGGAATTGAGCTCA
TAGGAGCTCAATTTTATTGGCATTGCTGTTGTTGGATGGTTAAAG
GGGTGGTATCCCTTTTCTCAG

In some embodiments, the targeted site may be 10 to 50 bps (e.g., 10 to 40, 10 to 30, 10 to 20, 15 to 30, 15 to 25, or 15 to 20 bps) in length. In some embodiments, the targeted strand in the targeted region is the sense strand of the gene. In other embodiments, the targeted strand in the targeted region is the antisense strand of the gene.

In some embodiments, an epigenetic editor as described herein may comprise one or more fusion proteins, wherein each fusion protein comprises a DNA-binding domain linked to one or more effector domains for epigenetic modification. In certain embodiments, where the DNA-binding domain is a polynucleotide guided DNA-binding domain, the epigenetic editor may further comprise one or more guide polynucleotides. DNA-binding domains, effector domains, and guide polynucleotides of an epigenetic editor as described herein may be selected, e.g., from those described below, in any functional combination.

The epigenetic editors described herein may be expressed in a host cell transiently, or may be integrated in a genome of the host cell; such cells and their progeny are also contemplated by the present disclosure. Both transiently expressed and integrated epigenetic editors or components thereof can effect stable epigenetic modifications. For example, after introducing to a host cell an epigenetic editor described herein, the target gene in the host cell may be stably or permanently repressed or silenced. In some embodiments, expression of the target gene is reduced or silenced for at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, or for the entire lifetime of the cell or the subject carrying the cell, as compared to the level of expression in the absence of the epigenetic editor. The epigenetic modification may be inherited by the progeny of the host cells into which the epigenetic editor was introduced.

The present epigenetic editors may be introduced to a cell (e.g., a human T lymphocyte or a human NK cell) that is then introduced into a patient (e.g., a human patient) in need thereof.

### I. DNA-Binding Domains

An epigenetic editor described herein may comprise one or more DNA-binding domains that direct the effector domain(s) of the epigenetic editor to target sequences within or close to the CIITA gene locus. A DNA-binding domain as described herein may be, e.g., a polynucleotide guided DNA-binding domain, a zinc finger protein (ZFP) domain, a transcription activator like effector (TALE) domain, a meganuclease DNA-binding domain, and the like. Examples of DNA-binding domains can be found in U.S. Pat. No. 11,162,114, which is incorporated by refence herein in its entirety.

In some embodiments, a DNA-binding domain described herein is encoded by its native coding sequence. In other embodiments, the DNA-binding domain is encoded by a nucleotide sequence that has been codon-optimized for optimal expression in human cells.

### A. Polynucleotide Guided DNA-Binding Domains

In some embodiments, a DNA-binding domain herein may be a protein domain directed by a guide nucleic acid sequence (e.g., a guide RNA sequence) to a target site in the CIITA gene locus. In certain embodiments, the protein domain may be derived from a CRISPR-associated nuclease, such as a Class I or II CRISPR-associated nuclease. In some embodiments, the protein domain may be derived from a Cas nuclease such as a Type II, Type IIA, Type IIB, Type IIC, Type V, or Type VI Cas nuclease. In certain embodiments, the protein domain may be derived from a Class II Cas nuclease selected from Cas1, Cas1B, Cas2, Cas3, Cas4, Cas5, Cas6, Cas7, Cas8, Cas9, Cas10, Cas14a, Cas14b, Cas14c, CasX, CasY, CasPhi, C2c4, C2c8, C2c9, C2c10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, and homologues and modified versions thereof. “Derived from” is used to mean that the protein domain comprises the full polypeptide sequence of the parent protein, or comprises a variant thereof (e.g., with amino acid residue deletions, insertions, and/or substitutions). The variant retains the desired function of the parent protein (e.g., the ability to form a complex with the guide nucleic acid sequence and the target DNA).

In some embodiments, the CRISPR-associated protein domain may be a Cas9 domain described herein. Cas9 may, for example, refer to a polypeptide with at least about 85%, 90%, 91%, 92%, 93%, 94%, 95%, 96%, 97%, 98%, 99%, or 100% sequence identity and/or sequence similarity to a wildtype Cas9 polypeptide described herein. In some embodiments, said wildtype polypeptide is Cas9 from Streptococcus pyogenes (NCBI Ref. No. NC_002737.2 (SEQ ID NO: 1)) and/or UniProt Ref. No. Q99ZW2 (SEQ ID NO: 2). In some embodiments, said wildtype polypeptide is Cas9 from Staphylococcus aureus (SEQ ID NO: 3). In some embodiments, the CRISPR-associated protein domain is a Cpf1 domain or protein, or a polypeptide with at least about 85%, 90%, 91%, 92%, 93%, 94%, 95%, 96%, 97%, 98%, 99%, or 100% sequence identity and/or sequence similarity to a wildtype Cpf1 polypeptide described herein (e.g., Cpf1 from Franscisella novicida (UniProt Ref. No. U2UMQ6 or SEQ ID NO: 4). In certain embodiments, the CRISPR-associated protein domain may be a modified form of the wildtype protein comprising one or more amino acid residue changes such as a deletion, an insertion, or a substitution; a fusion or chimera; or any combination thereof.

Cas9 sequences and structures of variant Cas9 orthologs have been described for various organisms. Exemplary organisms from which a Cas9 domain herein can be derived include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, and Acaryochloris marina. Cas9 sequences also include those from the organisms and loci disclosed in Chylinski et al., RNA Biol. (2013) 10 (5): 726-37.

In some embodiments, the Cas9 domain is from Streptococcus pyogenes. In some embodiments, the Cas9 domain is from Staphylococcus aureus.

Other Cas domains are also contemplated for use in the epigenetic editors herein. These include, for example, those from CasX (Cas12E) (e.g., SEQ ID NO: 5), CasY (Cas12d) (e.g., SEQ ID NO: 6), Casφ (CasPhi) (e.g., SEQ ID NO: 7), Cas12f1 (Cas14a) (e.g., SEQ ID NO: 8), Cas12f2 (Cas14b) (e.g., SEQ ID NO: 9), Cas12f3 (Cas14c) (e.g., SEQ ID NO: 10), and C2c8 (e.g., SEQ ID NO: 11).

For epigenetic editing, the nuclease-derived protein domain (e.g., a Cas9 or Cpf1 domain) may have reduced or no nuclease activity through mutations such that the protein domain does not cleave DNA or has reduced DNA-cleaving activity while retaining the ability to complex with the guide nucleic acid sequence (e.g., guide RNA) and the target DNA. For example, the nuclease activity may be reduced by at least 30%, 35%, 40%, 45%, 50%, 55%, 60%, 65%, 70%, 75%, 80%, 85%, 90%, 95%, or 99% compared to the wildtype domain. In some embodiments, a CRISPR-associated protein domain described herein is catalytically inactive (“dead”). Examples of such domains include, for example, dCas9 (“dead” Cas9), dCpf1, ddCpf1, dCasPhi, ddCas12a, dLbCpf1, and dFnCpf1. A dCas9 protein domain, for example, may comprise one, two, or more mutations as compared to wildtype Cas9 that abrogate its nuclease activity. The DNA cleavage domain of Cas9 is known to include two subdomains: the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A (in RuvC1) and H840A (in HNH) completely inactivate the nuclease activity of SpCas9. SaCas9, similarly, may be inactivated by the mutations D10A and N580A. In some embodiments, the dCas9 comprises at least one mutation in the HNH subdomain and/or the RuvC1 subdomain that reduces or abrogates nuclease activity. In some embodiments, the dCas9 only comprises a RuvC1 subdomain, or only comprises an HNH subdomain. It is to be understood that any mutation that inactivates the RuvC1 and/or the HNH domain may be included in a dCas9 herein, e.g., insertion, deletion, or single or multiple amino acid substitution in the RuvC1 domain and/or the HNH domain.

In some embodiments, a dCas9 protein herein comprises a mutation at position(s) corresponding to position D10 (e.g., D10A), H840 (e.g., H840A), or both, of a wildtype SpCas9 sequence as numbered in the sequence provided at UniProt Accession No. Q99ZW2 (SEQ ID NO: 2). In particular embodiments, the dCas9 comprises the amino acid sequence of dSpCas9 (D10A and H840A) (SEQ ID NO: 12).

In some embodiments, a dCas9 protein as described herein comprises a mutation at position(s) corresponding to position D10 (e.g., D10A), N580 (e.g., N580A), or both, of a wildtype SaCas9 sequence (e.g., SEQ ID NO: 3). In particular embodiments, the dCas9 comprises the amino acid sequence of dSaCas9 (D10A and N580A) (SEQ ID NO: 13).

Additional suitable mutations that inactivate Cas9 will be apparent to those of skill in the art based on this disclosure and knowledge in the field and are within the scope of this disclosure. Such mutations may include, but are not limited to, D839A, N863A, and/or K603R in SpCas9. The present disclosure contemplates any mutations that reduce or abrogate the nuclease activity of any Cas9 described herein (e.g., mutations corresponding to any of the Cas9 mutations described herein).

A dCpf1 protein domain may comprise one, two, or more mutations as compared to wildtype Cpf1 that reduce or abrogate its nuclease activity. The Cpf1 protein has a RuvC-like endonuclease domain that is similar to the RuvC domain of Cas9, but does not have an HNH endonuclease domain, and the N-terminal of Cpf1 does not have the alpha-helical recognition lobe of Cas9. In some embodiments, the dCpf1 comprises one or more mutations corresponding to position D917A, E1006A, or D1255A as numbered in the sequence of the Francisella novicida Cpf1 protein (FnCpf1; SEQ ID NO: 4). In certain embodiments, the dCpf1 protein comprises mutations corresponding to D917A, E1006A, D1255A, D917A/E1006A, D917A/D1255A, E1006A/D1255A, or D917A/E1006A/D1255A, or corresponding mutation(s) in any of the Cpf1 amino acid sequences described herein. In some embodiments, the dCpf1 comprises a D917A mutation. In particular embodiments, the dCpf1 comprises the amino acid sequence of dFnCpf1 (SEQ ID NO: 14).

Further nuclease inactive CRISPR-associated protein domains contemplated herein include those from, for example, dNmeCas9 (e.g., SEQ ID NO: 15), dCjCas9 (e.g., SEQ ID NO: 16), dSt1Cas9 (e.g., SEQ ID NO: 17), dSt3Cas9 (e.g., SEQ ID NO: 18), dLbCpf1 (e.g., SEQ ID NO: 19), dAsCpf1 (e.g., SEQ ID NO: 20), denAsCpf1 (e.g., SEQ ID NO: 21), dHFAsCpf1 (e.g., SEQ ID NO: 22), dRVRAsCpf1 (e.g., SEQ ID NO: 23), dRRAsCpf1 (e.g., SEQ ID NO: 24), dCasX (e.g., SEQ ID NO: 25), and dCasPhi (e.g., SEQ ID NO: 26).

In some embodiments, a Cas9 domain described herein may be a high fidelity Cas9 domain, e.g., comprising one or more mutations that decrease electrostatic interactions between the Cas9 domain and the sugar-phosphate backbone of DNA to confer increased target binding specificity. In certain embodiments, the high fidelity Cas9 domain may be nuclease inactive as described herein.

A CRISPR-associated protein domain described herein may recognize a protospacer adjacent motif (PAM) sequence in a target gene. A “PAM” sequence is typically a 2 to 6 bp DNA sequence immediately following the sequence targeted by the CRISPR-associated protein domain. The PAM sequence is required for CRISPR protein binding and cleavage but is not part of the target sequence. The CRISPR-associated protein domain may either recognize a naturally occurring or canonical PAM sequence or may have altered PAM specificity. CRISPR-associated protein domains that bind to non-canonical PAM sequences have been described in the art. For example, Cas9 domains that bind non-canonical PAM sequences have been described in Kleinstiver et al., Nature (2015) 523 (7561): 481-5 and Kleinstiver et al., Nat Biotechnol. (2015) 33:1293-8. Such Cas9 domains may include, for example, those from “VRER” SpCas9, “EQR” SpCas9, “VQR” SpCas9, “SpG Cas9,” “SpRYCas9,” and “KKH” SaCas9. Nuclease inactive versions of these Cas9 domains are also contemplated, such as nuclease inactive VRER SpCas9 (e.g., SEQ ID NO: 27), nuclease inactive EQR SpCas9 (e.g., SEQ ID NO: 28), nuclease inactive VQR SpCas9 (e.g., SEQ ID NO: 29), nuclease inactive SpG Cas9 (e.g., SEQ ID NO: 30), nuclease inactive SpRY Cas9 (e.g., SEQ ID NO: 31), and nuclease inactive KKH SaCas9 (e.g., SEQ ID NO: 32). Another example is the Cas9 of Francisella novicida engineered to recognize 5′-YG-3′ (where “Y” is a pyrimidine).

Additional suitable CRISPR-associated proteins, orthologs, and variants, including nuclease inactive variants and sequences, will be apparent to those of skill in the art based on this disclosure.

Guide RNAs that can be used in conjunction with the CRISPR-associated protein domains herein are further described in Section II below.

### B. Zinc Finger Protein Domains

In some embodiments, the DNA-binding domain of an epigenetic editor described herein comprises a zinc finger protein (ZFP) domain (or “ZF domain” as used herein). ZFPs are proteins having at least one zinc finger, and bind to DNA in a sequence-specific manner. A “zinc finger” (ZF) or “zinc finger motif” (ZF motif) refers to a polypeptide domain comprising a beta-beta-alpha (ββα)-protein fold stabilized by a zinc ion. A ZF binds from two to four base pairs of nucleotides, typically three or four base pairs (contiguous or noncontiguous). Each ZF typically comprises approximately 30 amino acids. ZFP domains may contain multiple ZFs that make tandem contacts with their target nucleic acid sequence. A tandem array of ZFs may be engineered to generate artificial ZFPs that bind desired nucleic acid targets. ZFPs may be rationally designed by using databases comprising triplet (or quadruplet) nucleotide sequences and individual ZF amino acid sequences, in which each triplet or quadruplet nucleotide sequence is associated with one or more amino acid sequences of ZFs that bind the particular triplet or quadruplet sequence. See, e.g., U.S. Pat. Nos. 6,453,242, 6,534,261, and 8,772,453.

ZFPs are widespread in eukaryotic cells, and may belong to, e.g., C2H2 class, CCHC class, PHD class, or RING class. An exemplary motif characterizing one class of these proteins (C2H2 class) is -Cys-(X)₂₋₄-Cys-(EAAAKX)₁₂-His-(X)₃₋₅-His-(SEQ ID NO: 657), where X is any independently chosen amino acid. In some embodiments, a ZFP domain herein may comprise a ZF array comprising sequential C2H2-ZFs each contacting three or more sequential nucleotides.

A ZFP domain of an epigenetic editor described herein may include 2, 3, 4, 5, 6, 7, 8, 9, 10, 11, 12, or more ZFs. The ZFP domain may include an array of two-finger or three-finger units, e.g., 3, 4, 5, 6, 7, 8, 9 or 10 or more units, wherein each unit binds a subsite in the target sequence. In some embodiments, a ZFP domain comprising at least three ZFs recognizes a target DNA sequence of 9 or 10 nucleotides. In some embodiments, a ZFP domain comprising at least four ZFs recognizes a target DNA sequence of 12 to 14 nucleotides. In some embodiments, a ZFP domain comprising at least six ZFs recognizes a target DNA sequence of 18 to 21 nucleotides.

In some embodiments, ZFs in a ZFP domain described herein are connected via peptide linkers. The peptide linkers may be, e.g., 2, 3, 4, 5, 6, 7, 8, 9, 10, 11, 12, 13, 14, 15, 16, 17, 18, 19, 20, or more amino acids in length. In some embodiments, a linker comprises 5 or more amino acids. In some embodiments, a linker comprises 7-17 amino acids. The linker may be flexible or rigid.

In some embodiments a zinc finger array may have the sequence:

(SEQ ID NO: 650)
SRPGERPFQCRICMRNFSXXXXXXXHXXTHTGEKPFQCRICMRN
FSXXXXXXXHXXTH[linker]FQCRICMRNFSXXXXXXXHXXT
HTGEKPFQCRICMRNFSXXXXXXXHXXTH[linker]PFQCRIC
MRNFSXXXXXXXHXXTHTGEKPFQCRICMRNFSXXXXXXXHXXT
HLRGS,

or a sequence at least 70%, 75%, 80%, 85%, 90%, 95%, 96%, 97%, 98%, or 99% identical thereto, where “XXXXXXX” represents the amino acids of the ZF recognition helix, which confers DNA-binding specificity upon the zinc finger; each X may be independently chosen. In the above sequence, “XX” in italics may be TR, LR or LK, and “[linker]” represents a linker sequence. In some embodiments, the linker sequence is TGSQKP (SEQ ID NO: 651); this linker may be used when sub-sites targeted by the ZFs are adjacent. In some embodiments, the linker sequence is TGGGGSQKP (SEQ ID NO: 652); this linker may be used when there is a base between the sub-sites targeted by the zinc fingers. The two indicated linkers may be the same or different. In some embodiments, the linker sequence is a minimum of 5 amino acids in length. In some embodiments, the linker sequence is a maximum of 250 amino acids in length.

ZFP domains herein may contain arrays of two or more adjacent ZFs that are directly adjacent to one another (e.g., separated by a short (canonical) linker sequence), or are separated by longer, flexible or structured polypeptide sequences. In some embodiments, directly adjacent fingers bind to contiguous nucleic acid sequences, i.e., to adjacent trinucleotides/triplets. In some embodiments, adjacent fingers cross-bind between each other's respective target triplets, which may help to strengthen or enhance the recognition of the target sequence, and leads to the binding of overlapping sequences. In some embodiments, distant ZFs within the ZFP domain may recognize (or bind to) non-contiguous nucleotide sequences.

Exemplary CIITA target genomic sequences are shown in Table 1 below.

TABLE 1
ZFP Target Sequences Within CIITA

| ZF Target No. | CIITA Target Site | SEQ ID NO |
|---|---|---|
| ZFTAR001 | GAAAATGACAGGTGGGCC | 700 |
| ZFTAR002 | TTGGTGTGGGAGTAGGCA | 701 |
| ZFTAR003 | GGGTGTTCAGAAACAGAA | 702 |
| ZFTAR004 | GGGGAAGCTGAGGGCACG | 703 |
| ZFTAR005 | GGTCAGGGGACGGGGGAA | 704 |
| ZFTAR006 | GGCTGGGAAGGGTGTGCC | 705 |
| ZFTAR007 | GCCGGTGTGGTTGGGTGC | 706 |
| ZFTAR008 | TAAGTTGCTACGGGAAAG | 707 |
| ZFTAR009 | GTTGGAGGGGGAGAAGTC | 708 |
| ZFTAR010 | GAGGACCCAGCAGGAATC | 709 |
| ZFTAR011 | GACAGGTAGGACCCAGCA | 710 |
| ZFTAR012 | CTGGAATGGCAGGACCAG | 711 |
| ZFTAR013 | GGAGTGGAGTCTGGCAGA | 712 |
| ZFTAR014 | GCCAACGGTGTAGCTGCC | 713 |
| ZFTAR015 | GGAGTCTGAGAAAAGGGA | 714 |
| ZFTAR016 | GCATGAGACTTTGAGGTG | 715 |
| ZFTAR017 | GATACTGTTGACTGTAGAA | 716 |
| ZFTAR018 | GACTGTAGAAGGCTCTGAG | 717 |
| ZFTAR019 | GCAATTTGGTGTGGGAGTA | 718 |
| ZFTAR020 | GAAGTCCCCAGCAGAGGCT | 719 |
| ZFTAR021 | GAGGCATCTGGGCGGAGGG | 720 |
| ZFTAR022 | TGGGGAAGCTGAGGGCACG | 721 |
| ZFTAR023 | GCTGAGGGCACGAGGAGGG | 722 |
| ZFTAR024 | AGGGCACGAGGAGGGGCTG | 723 |
| ZFTAR025 | GGAGCTGCTGCCTGGCTGG | 724 |
| ZFTAR026 | TGGGGATGGGGGTGAGGAT | 725 |
| ZFTAR027 | TGGGGGTGAGGATGGGAAC | 726 |
| ZFTAR028 | GAGGATGGGAACAGGAGTC | 727 |
| ZFTAR029 | GCAGCTGGGGAGAGCTGGC | 728 |
| ZFTAR030 | GCTGGGGAGAGCTGGCGGA | 729 |
| ZFTAR031 | GGGGAGAGCTGGCGGATGC | 730 |
| ZFTAR032 | GACGGGGGAACAGATGGTG | 731 |
| ZFTAR033 | GAACAGATGGTGGGAAGAG | 732 |
| ZFTAR034 | GTTGGCTGGGAAGGGTGTG | 733 |
| ZFTAR035 | TAGGAACTGGAATTGAGCT | 734 |
| ZFTAR036 | GATGGTTAAAGGGGTGGTA | 735 |
| ZFTAR037 | GGTGTGGTTGGGTGCAGCC | 736 |
| ZFTAR038 | GCAGCCTTAAGTTGCTACG | 737 |
| ZFTAR039 | GCTACGGGAAAGTGTTGGA | 738 |
| ZFTAR040 | GGAGGGGGAGAAGTCAGAG | 739 |
| ZFTAR041 | GGGGGAGAAGTCAGAGGTA | 740 |
| ZFTAR042 | GGAGGACCCAGCAGGAATC | 741 |
| ZFTAR043 | GTGGTATCAAGGAGGAAGT | 742 |
| ZFTAR044 | GTAGTCAGATGGTTTGGGG | 743 |
| ZFTAR045 | GAGAGCCTCTGCTGGGGAC | 744 |
| ZFTAR046 | ACAGAAAGTCTGTTGGGGG | 745 |
| ZFTAR047 | GAATTTGGTGAAGACAGAA | 746 |
| ZFTAR048 | GTGGACTGAATTTGGTGAA | 747 |
| ZFTAR049 | GCAACGCATTGTGTAGGAA | 748 |
| ZFTAR050 | TAGGACCCAGCAGGGCGTG | 749 |
| ZFTAR051 | GACAGGTAGGACCCAGCAG | 750 |
| ZFTAR052 | TGGGTATGGAGTGGAGTCT | 751 |
| ZFTAR053 | GGGGAGGGCAGCATCCGCC | 752 |
| ZFTAR054 | GTTGACTGTAGAAGGCTCTG | 753 |
| ZFTAR055 | GAGGCTGTGTGCTTCTGAG | 754 |

In some embodiments, the ZFP domain of the present epigenetic editor binds to a target sequence selected from any one of SEQ ID NOs: 700-754. The ZF may comprise the ZF framework sequence of SEQ ID NO: 650, or any other ZF framework known in the art.

### C. TALEs

In some embodiments, the DNA-binding domain of an epigenetic editor described herein comprises a transcription activator-like effector (TALE) domain. The DNA-binding domain of a TALE comprises a highly conserved sequence of about 33-34 amino acids, with a repeat variable di-residue (RVD) at positions 12 and 13 that is central to the recognition of specific nucleotides. TALEs can be engineered to bind practically any desired DNA sequence. Methods for programming TALEs are known in the art. For example, such methods are described in Carroll et al., Genet Soc Amer. (2011) 188 (4): 773-82; Miller et al., Nat Biotechnol. (2007) 25 (7): 778-85; Christian et al., Genetics (2008) 186 (2): 757-61; Li et al., Nucl Acids Res. (2010) 39 (1): 359-72; and Moscou et al., Science (2009) 326 (5959): 1501.

### D. Other DNA-Binding Domains

Other DNA-binding domains are contemplated for the epigenetic editors described herein. In some embodiments, the DNA-binding domain comprises an argonaute protein domain, e.g., from Natronobacterium gregoryi (NgAgo). NgAgo is a ssDNA-guided endonuclease that is guided to its target site by 5′ phosphorylated ssDNA (gDNA), where it produces double-strand breaks. In contrast to Cas9, the NgAgo-gDNA system does not require a protospacer-adjacent motif (PAM). Thus, using a nuclease inactive NgAgo (dNgAgo) can greatly expand the bases that may be targeted. The characterization and use of NgAgo have been described, e.g., in Gao et al., Nat Biotechnol. (2016) 34 (7): 768-73; Swarts et al., Nature (2014) 507 (7491): 258-61; and Swarts et al., Nucl Acids Res. (2015) 43 (10): 5120-9.

In some embodiments, the DNA-binding domain comprises an inactivated nuclease, for example, an inactivated meganuclease. Additional non-limiting examples of DNA-binding domains include tetracycline-controlled repressor (tetR) DNA-binding domains, leucine zippers, helix-loop-helix (HLH) domains, helix-turn-helix domains, β-sheet motifs, steroid receptor motifs, bZIP domains homeodomains, and AT-hooks.

### II. Guide Polynucleotides

Epigenetic editors described herein that comprise a polynucleotide guided DNA-binding domain may also include a guide polynucleotide that is capable of forming a complex with the DNA-binding domain. The guide polynucleotide may comprise RNA, DNA, or a mixture of both. For example, where the polynucleotide guided DNA-binding domain is a CRISPR-associated protein domain, the guide polynucleotide may be a guide RNA (gRNA). A “guide RNA” or “gRNA” refers to a nucleic acid that is able to hybridize to a target sequence and direct binding of the CRISPR-Cas complex to the target sequence. Methods of using guide polynucleotide sequences with programmable DNA-binding proteins (e.g., CRISPR-associated protein domains) for site-specific DNA targeting (e.g., to modify a genome) are known in the art.

A guide polynucleotide sequence (e.g., a gRNA sequence) may comprise two parts: 1) a nucleotide sequence comprising a “targeting sequence” that is complementary to a target nucleic acid sequence (“target sequence”), e.g., to a nucleic acid sequence comprised in a genomic target site; and 2) a nucleotide sequence that binds a polynucleotide guided DNA-binding domain (e.g., a CRISPR-Cas protein domain). The nucleotide sequence in 1) may comprise a targeting sequence that is 100% complementary to a genomic nucleic acid sequence, e.g., a nucleic acid sequence comprised in a genomic target site, and thus may hybridize to the target nucleic acid sequence. The nucleotide sequence in 1) may be referred to as, e.g., a crispr RNA, or crRNA. The nucleotide sequence in 2) may be referred to as a scaffold sequence of a guide nucleic acid, e.g., a tracrRNA, or an activating region of a guide nucleic acid, and may comprise a stem-loop structure. Parts 1) and 2) as described above may be fused to form one single guide (e.g., a single guide RNA, or sgRNA), or may be on two separate nucleic acid molecules. In some embodiments, a guide polynucleotide comprises parts 1) and 2) connected by a linker. In some embodiments, a guide polynucleotide comprises parts 1) and 2) connected by a non-nucleic acid linker, for example, a peptide linker or a chemical linker.

Part 2 (the scaffold sequence) of a guide polynucleotide as described herein may be, for example, as described in Jinek et al., Science (2012) 337:816-21; U.S. Patent Publication 2016/0208288; or U.S. Patent Publication 2016/0200779. Variants of part 2) are also contemplated by the present disclosure. For example, the tetraloop and stem loop of a gRNA scaffold (tracrRNA) sequence may be modified to include RNA aptamers, which can be bound by specific protein domains. In some embodiments, such modified gRNAs can be used to facilitate the recruitment of repressive or activating domains fused to the protein-interacting RNA aptamers.

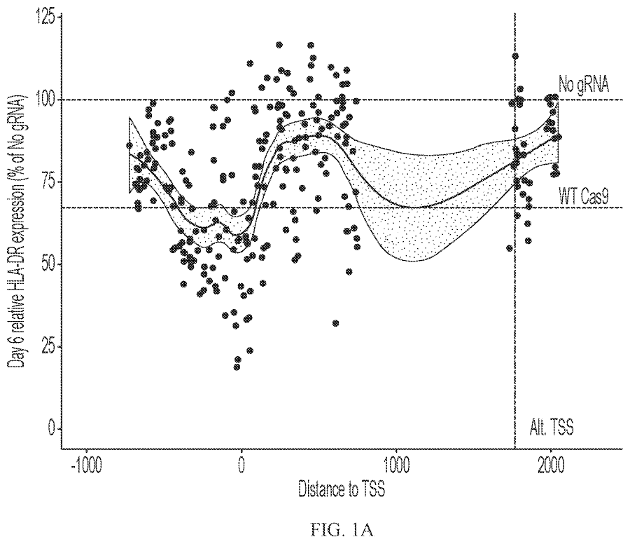

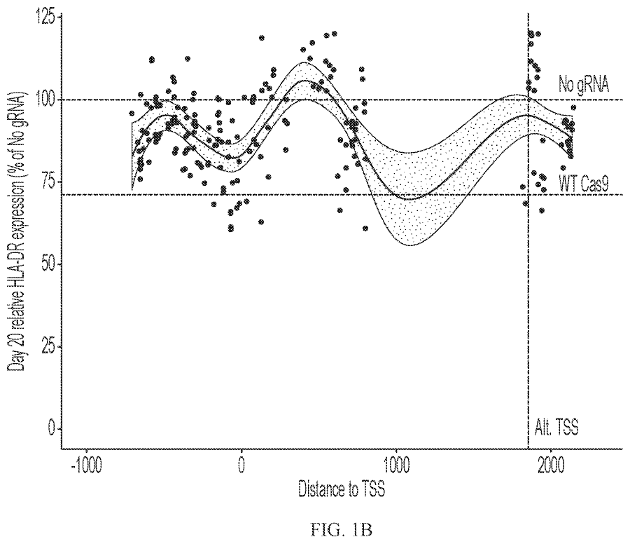

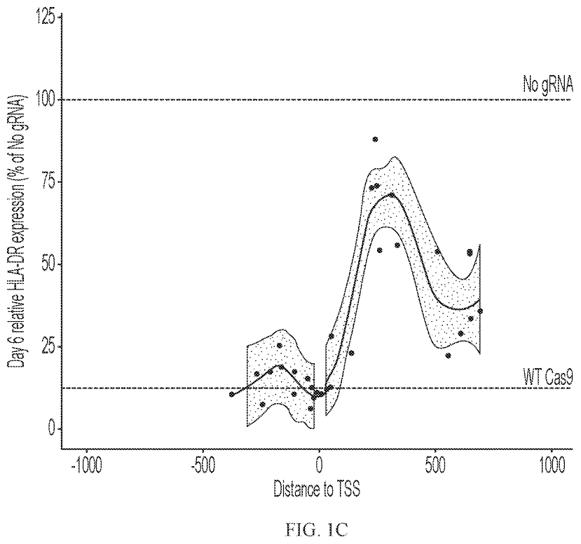

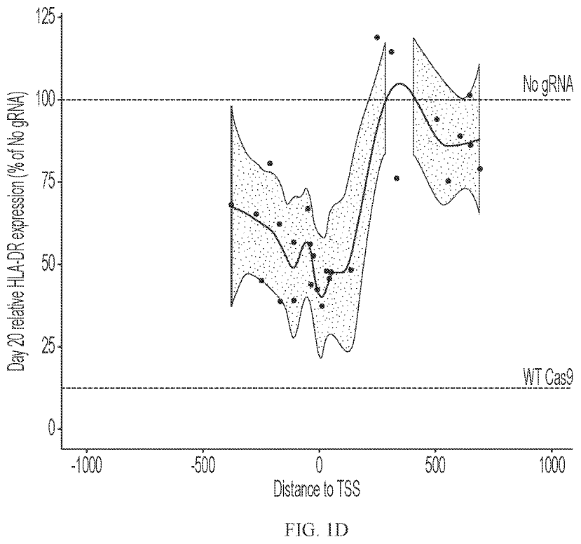

A gRNA as provided herein typically comprises a targeting domain and a binding domain. The targeting domain (also termed “targeting sequence”) may comprise a nucleic acid sequence that binds to a target site, e.g., to a genomic nucleic acid molecule within a cell. The target site may be a double-stranded DNA sequence comprising a PAM sequence as well as the target sequence, which is located on the same strand as, and directly adjacent to, the PAM sequence. The targeting domain of the gRNA may comprise an RNA sequence that corresponds to the target sequence, i.e., it resembles the sequence of the target domain, sometimes with one or more mismatches, but typically comprising an RNA sequence instead of a DNA sequence. The targeting domain of the gRNA thus may base pair (in full or partial complementarity) with the sequence of the double-stranded target site that is complementary to the target sequence, and thus with the strand complementary to the strand that comprises the PAM sequence. It will be understood that the targeting domain of the gRNA typically does not include a sequence that resembles the PAM sequence. It will further be understood that the location of the PAM may be 5′ or 3′ of the target sequence, depending on the nuclease employed. For example, the PAM is typically 3′ of the target sequence for Cas9 nucleases, and 5′ of the target sequence for Cas12a nucleases. For an illustration of the location of the PAM and the mechanism of gRNA binding to a target site, see, e.g., FIG. 1 of Vanegas et al., Fungal Biol Biotechnol. (2019) 6:6, which is incorporated by reference herein. For additional illustration and description of the mechanism of gRNA targeting of an RNA-guided nuclease to a target site, see Fu et al., Nat Biotechnol (2014) 32 (3): 279-84 and Sternberg et al., Nature (2014) 507 (7490): 62-7, each incorporated herein by reference.

In some embodiments, the targeting domain sequence comprises between 17 and 30 nucleotides and corresponds fully to the target sequence (i.e., without any mismatch nucleotides). In some embodiments, however, the targeting domain sequence may comprise one or more, but typically not more than 4, mismatches, e.g., 1, 2, 3, or 4 mismatches. As the targeting domain is part of gRNA, which is an RNA molecule, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target sequence (and thus base pairs with full complementarity with the DNA strand complementary to the strand comprising the target sequence and PAM) is provided below:

[ target domain (DNA) ][ PAM ]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
| | | | | | | | | | | | | | | | | | | | | |
5′-N-N-N-N-N-N-N-N-N-N-N-N-N-N-N-N-N-N-N-N-N-N-[ gRNA scaffold]-3′ (RNA)
[ target domain ( RNA) ][ binding domain ]

An exemplary illustration of a Cas12a target site, comprising a 22 nucleotide target domain, and a TTN PAM sequence, as well as of a gRNA comprising a targeting domain that fully corresponds to the target sequence (and thus base pairs with full complementarity with the DNA strand complementary to the strand comprising the target sequence and PAM) is provided below:

```
                      [ PAM ][ target domain ( DNA) ]
                      5′-T-T-N-N-N-N-N-N-N-N-N-N-N-N-N-N-N-N-N-N-N-N-N-N-N-3′ (DNA)
                      3′-A-A-N-N-N-N-N-N-N-N-N-N-N-N-N-N-N-N-N-N-N-N-N-N-N-5′ (DNA)
                                        | | | | | | | | | | | | | | | | | | | | | |
5′- [gRNA scaffold]-N-N-N-N-N-N-N-N-N-N-N-N-N-N-N-N-N-N-N-N-N-N-3′ (RNA)
[ binding domain ][ targeting domain ( RNA) ]
```

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In certain embodiments, the targeting domain fully corresponds, without mismatch, to a target sequence provided herein, or a part thereof. In some embodiments, the targeting domain of a gRNA provided herein comprises 1 mismatch relative to a target sequence provided herein. In some embodiments, the targeting domain comprises 2 mismatches relative to the target sequence. In some embodiments, the target domain comprises 3 mismatches relative to the target sequence.

Methods for designing, selecting, and validating gRNAs are described herein and known in the art. Software tools can be used to optimize the gRNAs corresponding to a target DNA sequence, e.g., to minimize total off-target activity across the genome. For example, DNA sequence searching algorithms can be used to identify a target sequence in crRNAs of a gRNA for use with Cas9. Exemplary gRNA design tools include the ones described in Bae et al., Bioinformatics (2014) 30:1473-5.

Guide polynucleotides (e.g., gRNAs) described herein may be of various lengths. In some embodiments, the length of the spacer or targeting sequence depends on the CRISPR-associated protein component of the epigenetic editor system used. For example, Cas proteins from different bacterial species have varying optimal targeting sequence lengths. Accordingly, the spacer sequence may comprise, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In some embodiments, the spacer comprises 10-24, 11-20, 11-16, 18-24, 19-21, or 20 nucleotides in length. In some embodiments, a guide polynucleotide (e.g., gRNA) is from 15-100 (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotides in length and comprises a spacer sequence of at least 10 (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) contiguous nucleotides complementary to the target sequence. In some embodiments, a guide polynucleotide described herein may be truncated, e.g., by 1, 2, 3, 4, 5, 10, 15, 20, 25, 30, 40, 50 or more nucleotides.

In certain embodiments, the 3′ end of the CIITA target sequence is immediately adjacent to a PAM sequence (e.g., a canonical PAM sequence such as NGG for SpCas9). The degree of complementarity between the targeting sequence of the guide polynucleotide (e.g., the spacer sequence of a gRNA) and the target sequence may be at least 90%, 91%, 92%, 93%, 94%, 95%, 96%, 97%, 98%, 99%, or 100%. In particular embodiments, the targeting and the target sequence may be 100% complementary. In other embodiments, the targeting sequence and the target sequence may contain, e.g., 1, 2, 3, 4, 5, 6, 7, 8, 9, or 10 mismatches.

A guide polynucleotide (e.g., gRNA) may be modified with, for example, chemical alterations and synthetic modifications. A modified gRNA, for instance, can include an alteration or replacement of one or both of the non-linking phosphate oxygens and/or of one or more of the linking phosphate oxygens in the phosphodiester backbone linkage, an alteration of the ribose sugar (e.g., of the 2′ hydroxyl on the ribose sugar), an alteration of the phosphate moiety, modification or replacement of a naturally occurring nucleobase, modification or replacement of the ribose-phosphate backbone, modification of the 3′ end and/or 5′ end of the oligonucleotide, replacement of a terminal phosphate group or conjugation of a moiety, cap, or linker, or any combination thereof.

In some embodiments, one or more ribose groups of the gRNA may be modified. Examples of chemical modifications to the ribose group include, but are not limited to, 2′-O-methyl(2′-OMe), 2′-fluoro (2′-F), 2′-deoxy, 2′-O-(2-methoxyethyl) (2′-MOE), 2′-NH2, 2′-O-allyl, 2′-O-ethylamine, 2′-O-cyanoethyl, 2′-O-acetalester, or a bicyclic nucleotide such as locked nucleic acid (LNA), 2′-(5-constrained ethyl (S-cEt)), constrained MOE, or 2′-0,4′-C-aminomethylene bridged nucleic acid (2′,4′-BNANC). 2′-O-methyl modification and/or 2′-fluoro modification may increase binding affinity and/or nuclease stability of the gRNA oligonucleotides.

In some embodiments, one or more phosphate groups of the gRNA may be chemically modified. Examples of chemical modifications to a phosphate group include, but are not limited to, a phosphorothioate (PS), phosphonoacetate (PACE), thiophosphonoacetate (thioPACE), amide, triazole, phosphonate, and phosphotriester modification. In some embodiments, a guide polynucleotide described herein may comprise one, two, three, or more PS linkages at or near the 5′ end and/or the 3′ end; the PS linkages may be contiguous or noncontiguous.

In some embodiments, the gRNA herein comprises a mixture of ribonucleotides and deoxyribonucleotides and/or one or more PS linkages.

In some embodiments, one or more nucleobases of the gRNA may be chemically modified. Examples of chemically modified nucleobases include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, and nucleobases with halogenated aromatic groups. Chemical modifications can be made in the spacer region, the tracr RNA region, the stem loop, or any combination thereof.

Table 2 below lists exemplary gRNA target sequences for epigenetic modification of human CIITA, as well as the coordinates of the start positions of the targeted site on human chromosome 16 (SEQ: SEQ ID NO). The Table also shows the distance from the start coordinate to the TSS coordinate of the CIITA gene. Table 3 lists exemplary targeting sequences for the gRNAs.

TABLE 2
Exemplary Target Sequences of gRNAs Targeting CIITA

| | Chr. 16 | | gRNA Target Sequence | | TSS1 |
|---|---|---|---|---|---|
| gRNA No. | Strand | START | (DNA, 5′ to 3′) | SEQ | Distance |
| gRNA001 | + | 10876478 | GAGTGATCATATATTAGAAG | 755 | −724 |
| gRNA002 | − | 10876545 | GTCTCTGCCCATCCTCCTGG | 756 | −657 |
| gRNA003 | + | 10876605 | ATTATTTAGCATCACTTTGG | 757 | −597 |
| gRNA004 | + | 10876645 | GACTACAAGGGTACCATATT | 758 | −557 |
| gRNA005 | + | 10876738 | TATGATCTCCAGAGAAATTC | 759 | −464 |
| gRNA006 | + | 10876770 | TGTGGGAGTAGGCATGGTAG | 760 | −432 |
| gRNA007 | + | 10876831 | CTTGTCTTGAGGCATCTGGG | 761 | −371 |
| gRNA008 | − | 10876872 | ATCTGCCACCTTCTGCAGGA | 762 | −330 |
| gRNA009 | − | 10876962 | TTGCACAGAAAGTCTGTTGG | 763 | −240 |
| gRNA010 | + | 10877023 | TGAAATTAATTTCAGAGGTG | 764 | −179 |
| gRNA011 | + | 10877056 | AGGGAGTGTGGTAAAATTAG | 765 | −146 |
| gRNA012 | − | 10876520 | TCAAGGAGGAAGTCTCAGCT | 766 | −682 |
| gRNA013 | + | 10876548 | CCAGGAGGATGGGCAGAGAC | 767 | −654 |
| gRNA014 | + | 10876606 | TTATTTAGCATCACTTTGGC | 768 | −596 |
| gRNA015 | + | 10876646 | ACTACAAGGGTACCATATTT | 769 | −556 |
| gRNA016 | + | 10876739 | ATGATCTCCAGAGAAATTCA | 770 | −463 |
| gRNA017 | + | 10876801 | ATCTAAGAAGTCCCCAGCAG | 771 | −401 |
| gRNA018 | + | 10876834 | GTCTTGAGGCATCTGGGCGG | 772 | −368 |
| gRNA019 | − | 10876876 | CAATATCTGCCACCTTCTGC | 773 | −326 |
| gRNA020 | − | 10876963 | GTTGCACAGAAAGTCTGTTG | 774 | −239 |
| gRNA021 | + | 10877024 | GAAATTAATTTCAGAGGTGT | 775 | −178 |
| gRNA022 | + | 10877058 | GGGAGTGTGGTAAAATTAGA | 776 | −144 |
| gRNA023 | + | 10876530 | ACTTCCTCCTTGATACCACC | 777 | −672 |
| gRNA024 | − | 10876548 | CCAGTCTCTGCCCATCCTCC | 778 | −654 |
| gRNA025 | + | 10876632 | CCCCAAACCATCTGACTACA | 779 | −570 |
| gRNA026 | − | 10876658 | AAAGAGTGTTAACCCAAATA | 780 | −544 |
| gRNA027 | − | 10876746 | CAAATTGCCCTGAATTTCTC | 781 | −456 |
| gRNA028 | − | 10876812 | CAAGCTGAGAGCCTCTGCTG | 782 | −390 |
| gRNA029 | + | 10876835 | TCTTGAGGCATCTGGGCGGA | 783 | −367 |
| gRNA030 | + | 10876877 | CTGCAGAAGGTGGCAGATAT | 784 | −325 |
| gRNA031 | − | 10876964 | AGTTGCACAGAAAGTCTGTT | 785 | −238 |
| gRNA032 | + | 10877025 | AAATTAATTTCAGAGGTGTG | 786 | −177 |
| gRNA033 | + | 10877086 | GAAACAGAAATCTGACCGCT | 787 | −116 |
| gRNA034 | + | 10876533 | TCCTCCTTGATACCACCAGG | 788 | −669 |
| gRNA035 | + | 10876574 | AGACACTAACTTTCTCCCTA | 789 | −628 |
| gRNA036 | − | 10876632 | CCTTGTAGTCAGATGGTTTG | 790 | −570 |
| gRNA037 | + | 10876661 | TATTTGGGTTAACACTCTTT | 791 | −541 |
| gRNA038 | + | 10876747 | CAGAGAAATTCAGGGCAATT | 792 | −455 |
| gRNA039 | − | 10876813 | ACAAGCTGAGAGCCTCTGCT | 793 | −389 |
| gRNA040 | + | 10876847 | TGGGCGGAGGGCTATGATAC | 794 | −355 |
| gRNA041 | + | 10876884 | AGGTGGCAGATATTGGCAGC | 795 | −318 |
| gRNA042 | − | 10876965 | AAGTTGCACAGAAAGTCTGT | 796 | −237 |
| gRNA043 | + | 10877028 | TTAATTTCAGAGGTGTGGGG | 797 | −174 |
| gRNA044 | + | 10877087 | AAACAGAAATCTGACCGCTT | 798 | −115 |
| gRNA045 | − | 10876534 | TCCTCCTGGTGGTATCAAGG | 799 | −668 |
| gRNA046 | + | 10876575 | GACACTAACTTTCTCCCTAT | 800 | −627 |
| gRNA047 | + | 10876633 | CCCAAACCATCTGACTACAA | 801 | −569 |
| gRNA048 | + | 10876706 | TGTCTTGGGATGAAAATGAC | 802 | −496 |
| gRNA049 | + | 10876752 | AAATTCAGGGCAATTTGGTG | 803 | −450 |
| gRNA050 | − | 10876814 | GACAAGCTGAGAGCCTCTGC | 804 | −388 |
| gRNA051 | + | 10876864 | TACTGGCCCCATCCTGCAGA | 805 | −338 |
| gRNA052 | + | 10876895 | ATTGGCAGCTGGCACCAGTG | 806 | −307 |
| gRNA053 | + | 10876997 | ACCAAATTCAGTCCACAGTA | 807 | −205 |
| gRNA054 | + | 10877029 | TAATTTCAGAGGTGTGGGGA | 808 | −173 |
| gRNA055 | + | 10877088 | AACAGAAATCTGACCGCTTG | 809 | −114 |
| gRNA056 | − | 10876537 | CCATCCTCCTGGTGGTATCA | 810 | −665 |
| gRNA057 | − | 10876589 | AAATAATACTGACTCCCATA | 811 | −613 |
| gRNA058 | − | 10876633 | CCCTTGTAGTCAGATGGTTT | 812 | −569 |
| gRNA059 | + | 10876709 | CTTGGGATGAAAATGACAGG | 813 | −493 |
| gRNA060 | + | 10876753 | AATTCAGGGCAATTTGGTGT | 814 | −449 |
| gRNA061 | + | 10876820 | GAGGCTCTCAGCTTGTCTTG | 815 | −382 |
| gRNA062 | + | 10876867 | TGGCCCCATCCTGCAGAAGG | 816 | −335 |
| gRNA063 | − | 10876909 | TGATCACAATGGAACCGCAC | 817 | −293 |
| gRNA064 | − | 10877002 | TCCTTACTGTGGACTGAATT | 818 | −200 |
| gRNA065 | + | 10877036 | AGAGGTGTGGGGAGGGCTTA | 819 | −166 |
| gRNA066 | + | 10877100 | ACCGCTTGGGGCCACCTTGC | 820 | −102 |
| gRNA067 | + | 10876537 | CCTTGATACCACCAGGAGGA | 821 | −665 |
| gRNA068 | − | 10876590 | TAAATAATACTGACTCCCAT | 822 | −612 |
| gRNA069 | − | 10876634 | ACCCTTGTAGTCAGATGGTT | 823 | −568 |
| gRNA070 | + | 10876710 | TTGGGATGAAAATGACAGGT | 824 | −492 |
| gRNA071 | + | 10876759 | GGGCAATTTGGTGTGGGAGT | 825 | −443 |
| gRNA072 | + | 10876827 | TCAGCTTGTCTTGAGGCATC | 826 | −375 |
| gRNA073 | − | 10876870 | CTGCCACCTTCTGCAGGATG | 827 | −332 |
| gRNA074 | − | 10876920 | TTCAGAAATGATGATCACAA | 828 | −282 |
| gRNA075 | − | 10877009 | TTAATTTCACTTCCTTACTG | 829 | −193 |
| gRNA076 | + | 10877037 | GAGGTGTGGGGAGGGCTTAA | 830 | −165 |
| gRNA077 | + | 10876538 | CTTGATACCACCAGGAGGAT | 831 | −664 |
| gRNA078 | + | 10876602 | AGTATTATTTAGCATCACTT | 832 | −600 |
| gRNA079 | − | 10876639 | ATGGTACCCTTGTAGTCAGA | 833 | −563 |
| gRNA080 | − | 10876733 | ATTTCTCTGGAGATCATAAG | 834 | −469 |
| gRNA081 | + | 10876764 | ATTTGGTGTGGGAGTAGGCA | 835 | −438 |
| gRNA082 | + | 10876828 | CAGCTTGTCTTGAGGCATCT | 836 | −374 |
| gRNA083 | − | 10876871 | TCTGCCACCTTCTGCAGGAT | 837 | −331 |
| gRNA084 | + | 10876937 | TCTGAACGTCAGACTGTTGA | 838 | −265 |
| gRNA085 | + | 10877018 | GGAAGTGAAATTAATTTCAG | 839 | −184 |
| gRNA086 | + | 10877044 | GGGGAGGGCTTAAGGGAGTG | 840 | −158 |
| gRNA087 | − | 10877105 | CCCTGCAAGGTGGCCCCAAG | 841 | −97 |
| gRNA088 | + | 10877157 | TGCATGTTGGCTTAGCTTGG | 842 | −45 |
| gRNA089 | + | 10877220 | GAGGCTGTGTGCTTCTGAG | 843 | 18 |
| gRNA090 | + | 10877251 | GGCATCCTTGGGGAAGCTGA | 844 | 49 |
| gRNA091 | − | 10877289 | AGGCAGCAGCTCCCGGAGTC | 845 | 87 |
| gRNA092 | + | 10877336 | TGCCTGGCTCCACGCCCTGC | 846 | 134 |
| gRNA093 | + | 10877368 | GTCAGAGCCCCAAGGTAAAA | 847 | 166 |
| gRNA094 | + | 10877448 | ACAGAGAAACCATTCTGAAT | 848 | 246 |
| gRNA095 | + | 10877462 | CTGAATTGGGGATGGGGGTG | 849 | 260 |
| gRNA096 | + | 10877503 | GTCCTGCTGGGGCAGGCCAT | 850 | 301 |
| gRNA097 | − | 10877554 | GACCTGAAGTCTCCCTCCGG | 851 | 352 |
| gRNA098 | + | 10877101 | CCGCTTGGGGCCACCTTGCA | 852 | −101 |
| gRNA099 | + | 10877158 | GCATGTTGGCTTAGCTTGGC | 853 | −44 |
| gRNA100 | + | 10877220 | AGGCTGTGTGCTTCTGAGCT | 854 | 18 |
| gRNA101 | − | 10877256 | TCGTGCCCTCAGCTTCCCCA | 855 | 54 |
| gRNA102 | + | 10877291 | AGACTCCGGGAGCTGCTGCC | 856 | 89 |
| gRNA103 | + | 10877338 | GCCTGGCTCCACGCCCTGCT | 857 | 136 |
| gRNA104 | + | 10877372 | GAGCCCCAAGGTAAAAAGGC | 858 | 170 |
| gRNA105 | + | 10877449 | CAGAGAAACCATTCTGAATT | 859 | 247 |
| gRNA106 | + | 10877466 | ATTGGGGATGGGGGTGAGGA | 860 | 264 |
| gRNA107 | − | 10877505 | TTCCAATGGCCTGCCCCAGC | 861 | 303 |
| gRNA108 | − | 10877557 | GCTGACCTGAAGTCTCCCTC | 862 | 355 |
| gRNA109 | − | 10877111 | AAAAAACTCTCCCTGCAAGG | 863 | −91 |
| gRNA110 | + | 10877169 | TAGCTTGGCGGGCTCCCAAC | 864 | −33 |
| gRNA111 | + | 10877230 | CTTCTGAGCTGGGCATCCGA | 865 | 28 |
| gRNA112 | + | 10877258 | TTGGGGAAGCTGAGGGCACG | 866 | 56 |
| gRNA113 | + | 10877295 | TCCGGGAGCTGCTGCCTGGC | 867 | 93 |
| gRNA114 | − | 10877338 | ACCCAGCAGGGCGTGGAGCC | 868 | 136 |
| gRNA115 | + | 10877373 | AGCCCCAAGGTAAAAAGGCC | 869 | 171 |
| gRNA116 | + | 10877450 | AGAGAAACCATTCTGAATTG | 870 | 248 |
| gRNA117 | + | 10877467 | TTGGGGATGGGGGTGAGGAT | 871 | 265 |
| gRNA118 | − | 10877519 | AACTCTTTCACATCTTCCAA | 872 | 317 |
| gRNA119 | + | 10877561 | AGGGAGACTTCAGGTCAGCC | 873 | 359 |
| gRNA120 | − | 10877114 | TCAAAAAAACTCTCCCTGCA | 874 | −88 |
| gRNA121 | + | 10877177 | GCGGGCTCCCAACTGGTGAC | 875 | −25 |
| gRNA122 | + | 10877239 | TGGGCATCCGAAGGCATCCT | 876 | 37 |
| gRNA123 | + | 10877261 | GGGAAGCTGAGGGCACGAGG | 877 | 59 |
| gRNA124 | − | 10877296 | CCCAGCCAGGCAGCAGCTCC | 878 | 94 |
| gRNA125 | − | 10877345 | AGGTAGGACCCAGCAGGGCG | 879 | 143 |
| gRNA126 | − | 10877391 | GCTAAATTAAGATGCTTTCC | 880 | 189 |
| gRNA127 | + | 10877454 | AAACCATTCTGAATTGGGGA | 881 | 252 |
| gRNA128 | + | 10877473 | ATGGGGGTGAGGATGGGAAC | 882 | 271 |
| gRNA129 | + | 10877538 | AGTTGTCTATTTCCTTCCAC | 883 | 336 |
| gRNA130 | + | 10877568 | CTTCAGGTCAGCCAGGTGTC | 884 | 366 |
| gRNA131 | − | 10877141 | ACATGCAAAGAAACAAGTGA | 885 | −61 |
| gRNA132 | − | 10877183 | TCACTAACCAGTCACCAGTT | 886 | −19 |
| gRNA133 | + | 10877240 | GGGCATCCGAAGGCATCCTT | 887 | 38 |
| gRNA134 | + | 10877262 | GGAAGCTGAGGGCACGAGGA | 888 | 60 |
| gRNA135 | + | 10877296 | CCGGGAGCTGCTGCCTGGCT | 889 | 94 |
| gRNA136 | − | 10877350 | CTGACAGGTAGGACCCAGCA | 890 | 148 |
| gRNA137 | + | 10877407 | ATTTAGCGTGCAGTCTCAGC | 891 | 205 |
| gRNA138 | + | 10877455 | AACCATTCTGAATTGGGGAT | 892 | 253 |
| gRNA139 | + | 10877490 | AACAGGAGTCTGTGTCCTGC | 893 | 288 |
| gRNA140 | + | 10877541 | TGTCTATTTCCTTCCACCGG | 894 | 339 |
| gRNA141 | − | 10877579 | TGGTTCATACTCCAGACACC | 895 | 377 |
| gRNA142 | − | 10877142 | AACATGCAAAGAAACAAGTG | 896 | −60 |
| gRNA143 | − | 10877184 | ATCACTAACCAGTCACCAGT | 897 | −18 |
| gRNA144 | + | 10877241 | GGCATCCGAAGGCATCCTTG | 898 | 39 |
| gRNA145 | + | 10877263 | GAAGCTGAGGGCACGAGGAG | 899 | 61 |
| gRNA146 | − | 10877309 | ATTGTGTAGGAATCCCAGCC | 900 | 107 |
| gRNA147 | − | 10877351 | TCTGACAGGTAGGACCCAGC | 901 | 149 |
| gRNA148 | − | 10877431 | CTCTGTTTATCTGGAATGGC | 902 | 229 |
| gRNA149 | + | 10877456 | ACCATTCTGAATTGGGGATG | 903 | 254 |
| gRNA150 | + | 10877491 | ACAGGAGTCTGTGTCCTGCT | 904 | 289 |
| gRNA151 | + | 10877542 | GTCTATTTCCTTCCACCGGA | 905 | 340 |
| gRNA152 | + | 10877597 | AACCATGTATCAGCACCGAA | 906 | 395 |
| gRNA153 | + | 10877144 | TCACTTGTTTCTTTGCATGT | 907 | −58 |
| gRNA154 | + | 10877188 | CTGGTGACTGGTTAGTGATG | 908 | −14 |
| gRNA155 | − | 10877246 | AGCTTCCCCAAGGATGCCTT | 909 | 44 |
| gRNA156 | + | 10877278 | GAGGAGGGGCTGCCAGACTC | 910 | 76 |
| gRNA157 | + | 10877320 | TTCCTACACAATGCGTTGCC | 911 | 118 |
| gRNA158 | + | 10877360 | TCCTACCTGTCAGAGCCCCA | 912 | 158 |
| gRNA159 | − | 10877439 | GTTTCTCTGTTTATCTGGAA | 913 | 237 |
| gRNA160 | + | 10877457 | CCATTCTGAATTGGGGATGG | 914 | 255 |
| gRNA161 | + | 10877492 | CAGGAGTCTGTGTCCTGCTG | 915 | 290 |
| gRNA162 | − | 10877550 | TGAAGTCTCCCTCCGGTGGA | 916 | 348 |
| gRNA163 | + | 10877154 | CTTTGCATGTTGGCTTAGCT | 917 | −48 |
| gRNA164 | + | 10877200 | TAGTGATGAGGCTAGTGATG | 918 | −2 |
| gRNA165 | + | 10877250 | AGGCATCCTTGGGGAAGCTG | 919 | 48 |
| gRNA166 | + | 10877278 | AGGAGGGGCTGCCAGACTCC | 920 | 76 |
| gRNA167 | − | 10877322 | AGCCAGGCAACGCATTGTGT | 921 | 120 |
| gRNA168 | − | 10877361 | ACCTTGGGGCTCTGACAGGT | 922 | 159 |
| gRNA169 | − | 10877440 | GAATGGTTTCTCTGTTTATC | 923 | 238 |
| gRNA170 | − | 10877457 | CCCCCATCCCCAATTCAGAA | 924 | 255 |
| gRNA171 | + | 10877496 | AGTCTGTGTCCTGCTGGGGC | 925 | 294 |
| gRNA172 | + | 10877552 | TTCCACCGGAGGGAGACTTC | 926 | 350 |
| gRNA173 | − | 10877599 | AACCTTTCGGTGCTGATACA | 927 | 397 |
| gRNA174 | − | 10877671 | ACCTTGCTGTGGGCCGAGCC | 928 | 469 |
| gRNA175 | − | 10877704 | CCCCTCAGTATTTACCCAAA | 929 | 502 |
| gRNA176 | − | 10877751 | TCTGGGTATGGAGTGGAGTC | 930 | 549 |
| gRNA177 | + | 10877787 | CAAAATTGGAGTCAGCCTTG | 931 | 585 |
| gRNA178 | + | 10877827 | CAGCAGCTGGGGAGAGCTGG | 932 | 625 |
| gRNA179 | − | 10877864 | AAACAACACCATTAGGAAAC | 933 | 662 |
| gRNA180 | + | 10877885 | TTAAAAAGGGTCAGGGGACG | 934 | 683 |
| gRNA181 | − | 10877940 | CTGCCTTCAGAGCCGGTGCC | 935 | 738 |
| gRNA182 | + | 10878974 | GCCGCGGCCCCAGAGCTGGC | 936 | 1772 |
| gRNA183 | + | 10879002 | GAGGCCACCAGCAGCGCGCG | 937 | 1800 |
| gRNA184 | − | 10877612 | GTCTGACTTCTAGAACCTTT | 938 | 410 |
| gRNA185 | − | 10877681 | AGGCGAGAAGACCTTGCTGT | 939 | 479 |
| gRNA186 | + | 10877704 | CCCTTTGGGTAAATACTGAG | 940 | 502 |
| gRNA187 | − | 10877758 | CTGCTTCTCTGGGTATGGAG | 941 | 556 |
| gRNA188 | − | 10877802 | GGCTCAACAGCTACACCTCA | 942 | 600 |
| gRNA189 | − | 10877856 | CCATTAGGAAACTGGGGGGA | 943 | 654 |
| gRNA190 | + | 10877871 | CCTAATGGTGTTGTTTAAAA | 944 | 669 |
| gRNA191 | + | 10877886 | TAAAAAGGGTCAGGGGACGG | 945 | 684 |
| gRNA192 | + | 10877945 | CACCGGCTCTGAAGGCAGCA | 946 | 743 |
| gRNA193 | − | 10878975 | TCCCGCCAGCTCTGGGGCCG | 947 | 1773 |
| gRNA194 | + | 10879003 | AGGCCACCAGCAGCGCGCGC | 948 | 1801 |
| gRNA195 | + | 10877618 | GGTTCTAGAAGTCAGACTTT | 949 | 416 |
| gRNA196 | − | 10877682 | GAGGCGAGAAGACCTTGCTG | 950 | 480 |
| gRNA197 | − | 10877705 | ACCCCTCAGTATTTACCCAA | 951 | 503 |
| gRNA198 | + | 10877759 | CACTCCATACCCAGAGAAGC | 952 | 557 |
| gRNA199 | + | 10877814 | GCTGTTGAGCCCTCAGCAGC | 953 | 612 |
| gRNA200 | + | 10877856 | CCCTCCCCCCAGTTTCCTAA | 954 | 654 |
| gRNA201 | + | 10877872 | CTAATGGTGTTGTTTAAAAA | 955 | 670 |
| gRNA202 | + | 10877895 | TCAGGGGACGGGGGAACAGA | 956 | 693 |
| gRNA203 | − | 10877947 | TGCCATGCTGCCTTCAGAGC | 957 | 745 |
| gRNA204 | + | 10878977 | GCGGCCCCAGAGCTGGCGGG | 958 | 1775 |
| gRNA205 | − | 10879006 | GCTCCCGCGCGCGCTGCTGG | 959 | 1804 |
| gRNA206 | + | 10877619 | GTTCTAGAAGTCAGACTTTC | 960 | 417 |
| gRNA207 | + | 10877689 | AAGGTCTTCTCGCCTCCCTT | 961 | 487 |
| gRNA208 | + | 10877716 | ATACTGAGGGGTGCCTCTGC | 962 | 514 |
| gRNA209 | + | 10877760 | ACTCCATACCCAGAGAAGCA | 963 | 558 |
| gRNA210 | + | 10877815 | CTGTTGAGCCCTCAGCAGCT | 964 | 613 |
| gRNA211 | − | 10877857 | ACCATTAGGAAACTGGGGGG | 965 | 655 |
| gRNA212 | + | 10877877 | GGTGTTGTTTAAAAAGGGTC | 966 | 675 |
| gRNA213 | + | 10877898 | GGGGACGGGGGAACAGATGG | 967 | 696 |
| gRNA214 | + | 10878933 | CGGGGAGGTAGGATGACCAG | 968 | 1731 |
| gRNA215 | + | 10878978 | CGGCCCCAGAGCTGGCGGGA | 969 | 1776 |
| gRNA216 | + | 10879020 | CGCGGGAGCCCGGGGAACAG | 970 | 1818 |
| gRNA217 | + | 10877648 | GTCACTAACTCTCAGCATGC | 971 | 446 |
| gRNA218 | + | 10877690 | AGGTCTTCTCGCCTCCCTTT | 972 | 488 |
| gRNA219 | + | 10877720 | TGAGGGGTGCCTCTGCAGGA | 973 | 518 |
| gRNA220 | − | 10877763 | TTTCCCTGCTTCTCTGGGTA | 974 | 561 |
| gRNA221 | + | 10877816 | TGTTGAGCCCTCAGCAGCTG | 975 | 614 |
| gRNA222 | − | 10877860 | AACACCATTAGGAAACTGGG | 976 | 658 |
| gRNA223 | + | 10877878 | GTGTTGTTTAAAAAGGGTCA | 977 | 676 |
| gRNA224 | + | 10877899 | GGGACGGGGGAACAGATGGT | 978 | 697 |
| gRNA225 | − | 10878949 | GTCTGTGGCAGCTCGTCCGC | 979 | 1747 |
| gRNA226 | − | 10878981 | TCTCCCTCCCGCCAGCTCTG | 980 | 1779 |
| gRNA227 | + | 10879024 | GGAGCCCGGGGAACAGCGGT | 981 | 1822 |
| gRNA228 | + | 10877653 | TAACTCTCAGCATGCTGGCC | 982 | 451 |
| gRNA229 | − | 10877701 | CTCAGTATTTACCCAAAGGG | 983 | 499 |
| gRNA230 | + | 10877721 | GAGGGGTGCCTCTGCAGGAC | 984 | 519 |
| gRNA231 | − | 10877768 | TTTGGTTTCCCTGCTTCTCT | 985 | 566 |
| gRNA232 | − | 10877823 | CAGCTCTCCCCAGCTGCTGA | 986 | 621 |
| gRNA233 | − | 10877861 | CAACACCATTAGGAAACTGG | 987 | 659 |
| gRNA234 | + | 10877879 | TGTTGTTTAAAAAGGGTCAG | 988 | 677 |
| gRNA235 | + | 10877922 | AAGAGCACAGTGCAGACACC | 989 | 720 |
| gRNA236 | + | 10878958 | GAGCTGCCACAGACTTGCCG | 990 | 1756 |
| gRNA237 | − | 10878982 | CTCTCCCTCCCGCCAGCTCT | 991 | 1780 |
| gRNA238 | − | 10879028 | GTCACCTACCGCTGTTCCCC | 992 | 1826 |
| gRNA239 | + | 10877658 | CTCAGCATGCTGGCCTGGCT | 993 | 456 |
| gRNA240 | + | 10877702 | CTCCCTTTGGGTAAATACTG | 994 | 500 |
| gRNA241 | − | 10877729 | GCAGAGGTCCCGTCCTGCAG | 995 | 527 |
| gRNA242 | + | 10877773 | AGAAGCAGGGAAACCAAAAT | 996 | 571 |
| gRNA243 | − | 10877824 | CCAGCTCTCCCCAGCTGCTG | 997 | 622 |
| gRNA244 | − | 10877862 | ACAACACCATTAGGAAACTG | 998 | 660 |
| gRNA245 | + | 10877883 | GTTTAAAAAGGGTCAGGGGA | 999 | 681 |
| gRNA246 | + | 10877928 | ACAGTGCAGACACCTGGCAC | 1000 | 726 |
| gRNA247 | + | 10878970 | ACTTGCCGCGGCCCCAGAGC | 1001 | 1768 |
| gRNA248 | + | 10878983 | CCAGAGCTGGCGGGAGGGAG | 1002 | 1781 |
| gRNA249 | + | 10877670 | GCCTGGCTCGGCCCACAGCA | 1003 | 468 |
| gRNA250 | + | 10877703 | TCCCTTTGGGTAAATACTGA | 1004 | 501 |
| gRNA251 | − | 10877745 | TATGGAGTGGAGTCTGGCAG | 1005 | 543 |
| gRNA252 | − | 10877786 | CTCAAGGCTGACTCCAATTT | 1006 | 584 |
| gRNA253 | + | 10877824 | CCTCAGCAGCTGGGGAGAGC | 1007 | 622 |
| gRNA254 | − | 10877863 | AACAACACCATTAGGAAACT | 1008 | 661 |
| gRNA255 | + | 10877884 | TTTAAAAAGGGTCAGGGGAC | 1009 | 682 |
| gRNA256 | + | 10877937 | ACACCTGGCACCGGCTCTGA | 1010 | 735 |
| gRNA257 | + | 10878973 | TGCCGCGGCCCCAGAGCTGG | 1011 | 1771 |
| gRNA258 | − | 10878983 | CCTCTCCCTCCCGCCAGCTC | 1012 | 1781 |
| gRNA259 | − | 10879029 | GGTCACCTACCGCTGTTCCC | 1013 | 1827 |
| gRNA260 | − | 10879193 | CAGAGTCGTTGCGGGGATGC | 1014 | 1991 |
| gRNA261 | + | 10879227 | GGAGCCGGGAACCCGGAGCT | 1015 | 2025 |
| gRNA262 | − | 10879050 | AGGGGTTACAGAGGAGACTT | 1016 | 1848 |
| gRNA263 | + | 10879198 | ATCCCCGCAACGACTCTGCG | 1017 | 1996 |
| gRNA264 | − | 10879231 | CAAGCCAAGCTCCGGGTTCC | 1018 | 2029 |
| gRNA265 | + | 10879054 | AGTCTCCTCTGTAACCCCTA | 1019 | 1852 |
| gRNA266 | + | 10879199 | TCCCCGCAACGACTCTGCGC | 1020 | 1997 |
| gRNA267 | − | 10879238 | GGCACAGCAAGCCAAGCTCC | 1021 | 2036 |
| gRNA268 | + | 10879058 | TCCTCTGTAACCCCTAAGGT | 1022 | 1856 |
| gRNA269 | + | 10879206 | AACGACTCTGCGCGGGAACC | 1023 | 2004 |
| gRNA270 | − | 10879239 | GGGCACAGCAAGCCAAGCTC | 1024 | 2037 |
| gRNA271 | − | 10879059 | CCCGACCTTAGGGGTTACAG | 1025 | 1857 |
| gRNA272 | + | 10879212 | TCTGCGCGGGAACCAGGAGC | 1026 | 2010 |
| gRNA273 | + | 10879249 | GCTTGCTGTGCCCAGAGCTC | 1027 | 2047 |
| gRNA274 | + | 10879059 | CCTCTGTAACCCCTAAGGTC | 1028 | 1857 |
| gRNA275 | + | 10879213 | CTGCGCGGGAACCAGGAGCC | 1029 | 2011 |
| gRNA276 | − | 10879175 | GCTGGAAACGAGGTGTTTCC | 1030 | 1973 |
| gRNA277 | + | 10879220 | GGAACCAGGAGCCGGGAACC | 1031 | 2018 |
| gRNA278 | − | 10879185 | TTGCGGGGATGCTGGAAACG | 1032 | 1983 |
| gRNA279 | − | 10879224 | AGCTCCGGGTTCCCGGCTCC | 1033 | 2022 |

TABLE 3
Exemplary Targeting Domain Sequences of
gRNAs Targeting CIITA

| | gRNA Targeting | |
|---|---|---|
| gRNA No. | Sequence (5′ to 3′) | SEQ |
| gRNA001 | GAGUGAUCAUAUAUUAGAAG | 1034 |
| gRNA002 | GUCUCUGCCCAUCCUCCUGG | 1035 |
| gRNA003 | AUUAUUUAGCAUCACUUUGG | 1036 |
| gRNA004 | GACUACAAGGGUACCAUAUU | 1037 |
| gRNA005 | UAUGAUCUCCAGAGAAAUUC | 1038 |
| gRNA006 | UGUGGGAGUAGGCAUGGUAG | 1039 |
| gRNA007 | CUUGUCUUGAGGCAUCUGGG | 1040 |
| gRNA008 | AUCUGCCACCUUCUGCAGGA | 1041 |
| gRNA009 | UUGCACAGAAAGUCUGUUGG | 1042 |
| gRNA010 | UGAAAUUAAUUUCAGAGGUG | 1043 |
| gRNA011 | AGGGAGUGUGGUAAAAUUAG | 1044 |
| gRNA012 | UCAAGGAGGAAGUCUCAGCU | 1045 |
| gRNA013 | CCAGGAGGAUGGGCAGAGAC | 1046 |
| gRNA014 | UUAUUUAGCAUCACUUUGGC | 1047 |
| gRNA015 | ACUACAAGGGUACCAUAUUU | 1048 |
| gRNA016 | AUGAUCUCCAGAGAAAUUCA | 1049 |
| gRNA017 | AUCUAAGAAGUCCCCAGCAG | 1050 |
| gRNA018 | GUCUUGAGGCAUCUGGGCGG | 1051 |
| gRNA019 | CAAUAUCUGCCACCUUCUGC | 1052 |
| gRNA020 | GUUGCACAGAAAGUCUGUUG | 1053 |
| gRNA021 | GAAAUUAAUUUCAGAGGUGU | 1054 |
| gRNA022 | GGGAGUGUGGUAAAAUUAGA | 1055 |
| gRNA023 | ACUUCCUCCUUGAUACCACC | 1056 |
| gRNA024 | CCAGUCUCUGCCCAUCCUCC | 1057 |
| gRNA025 | CCCCAAACCAUCUGACUACA | 1058 |
| gRNA026 | AAAGAGUGUUAACCCAAAUA | 1059 |
| gRNA027 | CAAAUUGCCCUGAAUUUCUC | 1060 |
| gRNA028 | CAAGCUGAGAGCCUCUGCUG | 1061 |
| gRNA029 | UCUUGAGGCAUCUGGGCGGA | 1062 |
| gRNA030 | CUGCAGAAGGUGGCAGAUAU | 1063 |
| gRNA031 | AGUUGCACAGAAAGUCUGUU | 1064 |
| gRNA032 | AAAUUAAUUUCAGAGGUGUG | 1065 |
| gRNA033 | GAAACAGAAAUCUGACCGCU | 1066 |
| gRNA034 | UCCUCCUUGAUACCACCAGG | 1067 |
| gRNA035 | AGACACUAACUUUCUCCCUA | 1068 |
| gRNA036 | CCUUGUAGUCAGAUGGUUUG | 1069 |
| gRNA037 | UAUUUGGGUUAACACUCUUU | 1070 |
| gRNA038 | CAGAGAAAUUCAGGGCAAUU | 1071 |
| gRNA039 | ACAAGCUGAGAGCCUCUGCU | 1072 |
| gRNA040 | UGGGCGGAGGGCUAUGAUAC | 1073 |
| gRNA041 | AGGUGGCAGAUAUUGGCAGC | 1074 |
| gRNA042 | AAGUUGCACAGAAAGUCUGU | 1075 |
| gRNA043 | UUAAUUUCAGAGGUGUGGGG | 1076 |
| gRNA044 | AAACAGAAAUCUGACCGCUU | 1077 |
| gRNA045 | UCCUCCUGGUGGUAUCAAGG | 1078 |
| gRNA046 | GACACUAACUUUCUCCCUAU | 1079 |
| gRNA047 | CCCAAACCAUCUGACUACAA | 1080 |
| gRNA048 | UGUCUUGGGAUGAAAAUGAC | 1081 |
| gRNA049 | AAAUUCAGGGCAAUUUGGUG | 1082 |
| gRNA050 | GACAAGCUGAGAGCCUCUGC | 1083 |
| gRNA051 | UACUGGCCCCAUCCUGCAGA | 1084 |
| gRNA052 | AUUGGCAGCUGGCACCAGUG | 1085 |
| gRNA053 | ACCAAAUUCAGUCCACAGUA | 1086 |
| gRNA054 | UAAUUUCAGAGGUGUGGGGA | 1087 |
| gRNA055 | AACAGAAAUCUGACCGCUUG | 1088 |
| gRNA056 | CCAUCCUCCUGGUGGUAUCA | 1089 |
| gRNA057 | AAAUAAUACUGACUCCCAUA | 1090 |
| gRNA058 | CCCUUGUAGUCAGAUGGUUU | 1091 |
| gRNA059 | CUUGGGAUGAAAAUGACAGG | 1092 |
| gRNA060 | AAUUCAGGGCAAUUUGGUGU | 1093 |
| gRNA061 | GAGGCUCUCAGCUUGUCUUG | 1094 |
| gRNA062 | UGGCCCCAUCCUGCAGAAGG | 1095 |
| gRNA063 | UGAUCACAAUGGAACCGCAC | 1096 |
| gRNA064 | UCCUUACUGUGGACUGAAUU | 1097 |
| gRNA065 | AGAGGUGUGGGGAGGGCUUA | 1098 |
| gRNA066 | ACCGCUUGGGGCCACCUUGC | 1099 |
| gRNA067 | CCUUGAUACCACCAGGAGGA | 1100 |
| gRNA068 | UAAAUAAUACUGACUCCCAU | 1101 |
| gRNA069 | ACCCUUGUAGUCAGAUGGUU | 1102 |
| gRNA070 | UUGGGAUGAAAAUGACAGGU | 1103 |
| gRNA071 | GGGCAAUUUGGUGUGGGAGU | 1104 |
| gRNA072 | UCAGCUUGUCUUGAGGCAUC | 1105 |
| gRNA073 | CUGCCACCUUCUGCAGGAUG | 1106 |
| gRNA074 | UUCAGAAAUGAUGAUCACAA | 1107 |
| gRNA075 | UUAAUUUCACUUCCUUACUG | 1108 |
| gRNA076 | GAGGUGUGGGGAGGGCUUAA | 1109 |
| gRNA077 | CUUGAUACCACCAGGAGGAU | 1110 |
| gRNA078 | AGUAUUAUUUAGCAUCACUU | 1111 |
| gRNA079 | AUGGUACCCUUGUAGUCAGA | 1112 |
| gRNA080 | AUUUCUCUGGAGAUCAUAAG | 1113 |
| gRNA081 | AUUUGGUGUGGGAGUAGGCA | 1114 |
| gRNA082 | CAGCUUGUCUUGAGGCAUCU | 1115 |
| gRNA083 | UCUGCCACCUUCUGCAGGAU | 1116 |
| gRNA084 | UCUGAACGUCAGACUGUUGA | 1117 |
| gRNA085 | GGAAGUGAAAUUAAUUUCAG | 1118 |
| gRNA086 | GGGGAGGGCUUAAGGGAGUG | 1119 |
| gRNA087 | CCCUGCAAGGUGGCCCCAAG | 1120 |
| gRNA088 | UGCAUGUUGGCUUAGCUUGG | 1121 |
| gRNA089 | GAGGCUGUGUGCUUCUGAG | 1122 |
| gRNA090 | GGCAUCCUUGGGGAAGCUGA | 1123 |
| gRNA091 | AGGCAGCAGCUCCCGGAGUC | 1124 |
| gRNA092 | UGCCUGGCUCCACGCCCUGC | 1125 |
| gRNA093 | GUCAGAGCCCCAAGGUAAAA | 1126 |
| gRNA094 | ACAGAGAAACCAUUCUGAAU | 1127 |
| gRNA095 | CUGAAUUGGGGAUGGGGGUG | 1128 |
| gRNA096 | GUCCUGCUGGGGCAGGCCAU | 1129 |
| gRNA097 | GACCUGAAGUCUCCCUCCGG | 1130 |
| gRNA098 | CCGCUUGGGGCCACCUUGCA | 1131 |
| gRNA099 | GCAUGUUGGCUUAGCUUGGC | 1132 |
| gRNA100 | AGGCUGUGUGCUUCUGAGCU | 1133 |
| gRNA101 | UCGUGCCCUCAGCUUCCCCA | 1134 |
| gRNA102 | AGACUCCGGGAGCUGCUGCC | 1135 |
| gRNA103 | GCCUGGCUCCACGCCCUGCU | 1136 |
| gRNA104 | GAGCCCCAAGGUAAAAAGGC | 1137 |
| gRNA105 | CAGAGAAACCAUUCUGAAUU | 1138 |
| gRNA106 | AUUGGGGAUGGGGGUGAGGA | 1139 |
| gRNA107 | UUCCAAUGGCCUGCCCCAGC | 1140 |
| gRNA108 | GCUGACCUGAAGUCUCCCUC | 1141 |
| gRNA109 | AAAAAACUCUCCCUGCAAGG | 1142 |
| gRNA110 | UAGCUUGGCGGGCUCCCAAC | 1143 |
| gRNA111 | CUUCUGAGCUGGGCAUCCGA | 1144 |
| gRNA112 | UUGGGGAAGCUGAGGGCACG | 1145 |
| gRNA113 | UCCGGGAGCUGCUGCCUGGC | 1146 |
| gRNA114 | ACCCAGCAGGGCGUGGAGCC | 1147 |
| gRNA115 | AGCCCCAAGGUAAAAAGGCC | 1148 |
| gRNA116 | AGAGAAACCAUUCUGAAUUG | 1149 |
| gRNA117 | UUGGGGAUGGGGGUGAGGAU | 1150 |
| gRNA118 | AACUCUUUCACAUCUUCCAA | 1151 |
| gRNA119 | AGGGAGACUUCAGGUCAGCC | 1152 |
| gRNA120 | UCAAAAAAACUCUCCCUGCA | 1153 |
| gRNA121 | GCGGGCUCCCAACUGGUGAC | 1154 |
| gRNA122 | UGGGCAUCCGAAGGCAUCCU | 1155 |
| gRNA123 | GGGAAGCUGAGGGCACGAGG | 1156 |
| gRNA124 | CCCAGCCAGGCAGCAGCUCC | 1157 |
| gRNA125 | AGGUAGGACCCAGCAGGGCG | 1158 |
| gRNA126 | GCUAAAUUAAGAUGCUUUCC | 1159 |
| gRNA127 | AAACCAUUCUGAAUUGGGGA | 1160 |
| gRNA128 | AUGGGGGUGAGGAUGGGAAC | 1161 |
| gRNA129 | AGUUGUCUAUUUCCUUCCAC | 1162 |
| gRNA130 | CUUCAGGUCAGCCAGGUGUC | 1163 |
| gRNA131 | ACAUGCAAAGAAACAAGUGA | 1164 |
| gRNA132 | UCACUAACCAGUCACCAGUU | 1165 |
| gRNA133 | GGGCAUCCGAAGGCAUCCUU | 1166 |
| gRNA134 | GGAAGCUGAGGGCACGAGGA | 1167 |
| gRNA135 | CCGGGAGCUGCUGCCUGGCU | 1168 |
| gRNA136 | CUGACAGGUAGGACCCAGCA | 1169 |
| gRNA137 | AUUUAGCGUGCAGUCUCAGC | 1170 |
| gRNA138 | AACCAUUCUGAAUUGGGGAU | 1171 |
| gRNA139 | AACAGGAGUCUGUGUCCUGC | 1172 |
| gRNA140 | UGUCUAUUUCCUUCCACCGG | 1173 |
| gRNA141 | UGGUUCAUACUCCAGACACC | 1174 |
| gRNA142 | AACAUGCAAAGAAACAAGUG | 1175 |
| gRNA143 | AUCACUAACCAGUCACCAGU | 1176 |
| gRNA144 | GGCAUCCGAAGGCAUCCUUG | 1177 |
| gRNA145 | GAAGCUGAGGGCACGAGGAG | 1178 |
| gRNA146 | AUUGUGUAGGAAUCCCAGCC | 1179 |
| gRNA147 | UCUGACAGGUAGGACCCAGC | 1180 |
| gRNA148 | CUCUGUUUAUCUGGAAUGGC | 1181 |
| gRNA149 | ACCAUUCUGAAUUGGGGAUG | 1182 |
| gRNA150 | ACAGGAGUCUGUGUCCUGCU | 1183 |
| gRNA151 | GUCUAUUUCCUUCCACCGGA | 1184 |
| gRNA152 | AACCAUGUAUCAGCACCGAA | 1185 |
| gRNA153 | UCACUUGUUUCUUUGCAUGU | 1186 |
| gRNA154 | CUGGUGACUGGUUAGUGAUG | 1187 |
| gRNA155 | AGCUUCCCCAAGGAUGCCUU | 1188 |
| gRNA156 | GAGGAGGGGCUGCCAGACUC | 1189 |
| gRNA157 | UUCCUACACAAUGCGUUGCC | 1190 |
| gRNA158 | UCCUACCUGUCAGAGCCCCA | 1191 |
| gRNA159 | GUUUCUCUGUUUAUCUGGAA | 1192 |
| gRNA160 | CCAUUCUGAAUUGGGGAUGG | 1193 |
| gRNA161 | CAGGAGUCUGUGUCCUGCUG | 1194 |
| gRNA162 | UGAAGUCUCCCUCCGGUGGA | 1195 |
| gRNA163 | CUUUGCAUGUUGGCUUAGCU | 1196 |
| gRNA164 | UAGUGAUGAGGCUAGUGAUG | 1197 |
| gRNA165 | AGGCAUCCUUGGGGAAGCUG | 1198 |
| gRNA166 | AGGAGGGGCUGCCAGACUCC | 1199 |
| gRNA167 | AGCCAGGCAACGCAUUGUGU | 1200 |
| gRNA168 | ACCUUGGGGCUCUGACAGGU | 1201 |
| gRNA169 | GAAUGGUUUCUCUGUUUAUC | 1202 |
| gRNA170 | CCCCCAUCCCCAAUUCAGAA | 1203 |
| gRNA171 | AGUCUGUGUCCUGCUGGGGC | 1204 |
| gRNA172 | UUCCACCGGAGGGAGACUUC | 1205 |
| gRNA173 | AACCUUUCGGUGCUGAUACA | 1206 |
| gRNA174 | ACCUUGCUGUGGGCCGAGCC | 1207 |
| gRNA175 | CCCCUCAGUAUUUACCCAAA | 1208 |
| gRNA176 | UCUGGGUAUGGAGUGGAGUC | 1209 |
| gRNA177 | CAAAAUUGGAGUCAGCCUUG | 1210 |
| gRNA178 | CAGCAGCUGGGGAGAGCUGG | 1211 |
| gRNA179 | AAACAACACCAUUAGGAAAC | 1212 |
| gRNA180 | UUAAAAAGGGUCAGGGGACG | 1213 |
| gRNA181 | CUGCCUUCAGAGCCGGUGCC | 1214 |
| gRNA182 | GCCGCGGCCCCAGAGCUGGC | 1215 |
| gRNA183 | GAGGCCACCAGCAGCGCGCG | 1216 |
| gRNA184 | GUCUGACUUCUAGAACCUUU | 1217 |
| gRNA185 | AGGCGAGAAGACCUUGCUGU | 1218 |
| gRNA186 | CCCUUUGGGUAAAUACUGAG | 1219 |
| gRNA187 | CUGCUUCUCUGGGUAUGGAG | 1220 |
| gRNA188 | GGCUCAACAGCUACACCUCA | 1221 |
| gRNA189 | CCAUUAGGAAACUGGGGGGA | 1222 |
| gRNA190 | CCUAAUGGUGUUGUUUAAAA | 1223 |
| gRNA191 | UAAAAAGGGUCAGGGGACGG | 1224 |
| gRNA192 | CACCGGCUCUGAAGGCAGCA | 1225 |
| gRNA193 | UCCCGCCAGCUCUGGGGCCG | 1226 |
| gRNA194 | AGGCCACCAGCAGCGCGCGC | 1227 |
| gRNA195 | GGUUCUAGAAGUCAGACUUU | 1228 |
| gRNA196 | GAGGCGAGAAGACCUUGCUG | 1229 |
| gRNA197 | ACCCCUCAGUAUUUACCCAA | 1230 |
| gRNA198 | CACUCCAUACCCAGAGAAGC | 1231 |
| gRNA199 | GCUGUUGAGCCCUCAGCAGC | 1232 |
| gRNA200 | CCCUCCCCCCAGUUUCCUAA | 1233 |
| gRNA201 | CUAAUGGUGUUGUUUAAAAA | 1234 |
| gRNA202 | UCAGGGGACGGGGGAACAGA | 1235 |
| gRNA203 | UGCCAUGCUGCCUUCAGAGC | 1236 |
| gRNA204 | GCGGCCCCAGAGCUGGCGGG | 1237 |
| gRNA205 | GCUCCCGCGCGCGCUGCUGG | 1238 |
| gRNA206 | GUUCUAGAAGUCAGACUUUC | 1239 |
| gRNA207 | AAGGUCUUCUCGCCUCCCUU | 1240 |
| gRNA208 | AUACUGAGGGGUGCCUCUGC | 1241 |
| gRNA209 | ACUCCAUACCCAGAGAAGCA | 1242 |
| gRNA210 | CUGUUGAGCCCUCAGCAGCU | 1243 |
| gRNA211 | ACCAUUAGGAAACUGGGGGG | 1244 |
| gRNA212 | GGUGUUGUUUAAAAAGGGUC | 1245 |
| gRNA213 | GGGGACGGGGGAACAGAUGG | 1246 |
| gRNA214 | CGGGGAGGUAGGAUGACCAG | 1247 |
| gRNA215 | CGGCCCCAGAGCUGGCGGGA | 1248 |
| gRNA216 | CGCGGGAGCCCGGGGAACAG | 1249 |
| gRNA217 | GUCACUAACUCUCAGCAUGC | 1250 |
| gRNA218 | AGGUCUUCUCGCCUCCCUUU | 1251 |
| gRNA219 | UGAGGGGUGCCUCUGCAGGA | 1252 |
| gRNA220 | UUUCCCUGCUUCUCUGGGUA | 1253 |
| gRNA221 | UGUUGAGCCCUCAGCAGCUG | 1254 |
| gRNA222 | AACACCAUUAGGAAACUGGG | 1255 |
| gRNA223 | GUGUUGUUUAAAAAGGGUCA | 1256 |
| gRNA224 | GGGACGGGGGAACAGAUGGU | 1257 |
| gRNA225 | GUCUGUGGCAGCUCGUCCGC | 1258 |
| gRNA226 | UCUCCCUCCCGCCAGCUCUG | 1259 |
| gRNA227 | GGAGCCCGGGGAACAGCGGU | 1260 |
| gRNA228 | UAACUCUCAGCAUGCUGGCC | 1261 |
| gRNA229 | CUCAGUAUUUACCCAAAGGG | 1262 |
| gRNA230 | GAGGGGUGCCUCUGCAGGAC | 1263 |
| gRNA231 | UUUGGUUUCCCUGCUUCUCU | 1264 |
| gRNA232 | CAGCUCUCCCCAGCUGCUGA | 1265 |
| gRNA233 | CAACACCAUUAGGAAACUGG | 1266 |
| gRNA234 | UGUUGUUUAAAAAGGGUCAG | 1267 |
| gRNA235 | AAGAGCACAGUGCAGACACC | 1268 |
| gRNA236 | GAGCUGCCACAGACUUGCCG | 1269 |
| gRNA237 | CUCUCCCUCCCGCCAGCUCU | 1270 |
| gRNA238 | GUCACCUACCGCUGUUCCCC | 1271 |
| gRNA239 | CUCAGCAUGCUGGCCUGGCU | 1272 |
| gRNA240 | CUCCCUUUGGGUAAAUACUG | 1273 |
| gRNA241 | GCAGAGGUCCCGUCCUGCAG | 1274 |
| gRNA242 | AGAAGCAGGGAAACCAAAAU | 1275 |
| gRNA243 | CCAGCUCUCCCCAGCUGCUG | 1276 |
| gRNA244 | ACAACACCAUUAGGAAACUG | 1277 |
| gRNA245 | GUUUAAAAAGGGUCAGGGGA | 1278 |
| gRNA246 | ACAGUGCAGACACCUGGCAC | 1279 |
| gRNA247 | ACUUGCCGCGGCCCCAGAGC | 1280 |
| gRNA248 | CCAGAGCUGGGGGAGGGAG | 1281 |
| gRNA249 | GCCUGGCUCGGCCCACAGCA | 1282 |
| gRNA250 | UCCCUUUGGGUAAAUACUGA | 1283 |
| gRNA251 | UAUGGAGUGGAGUCUGGCAG | 1284 |
| gRNA252 | CUCAAGGCUGACUCCAAUUU | 1285 |
| gRNA253 | CCUCAGCAGCUGGGGAGAGC | 1286 |
| gRNA254 | AACAACACCAUUAGGAAACU | 1287 |
| gRNA255 | UUUAAAAAGGGUCAGGGGAC | 1288 |
| gRNA256 | ACACCUGGCACCGGCUCUGA | 1289 |
| gRNA257 | UGCCGCGGCCCCAGAGCUGG | 1290 |
| gRNA258 | CCUCUCCCUCCCGCCAGCUC | 1291 |
| gRNA259 | GGUCACCUACCGCUGUUCCC | 1292 |
| gRNA260 | CAGAGUCGUUGCGGGGAUGC | 1293 |
| gRNA261 | GGAGCCGGGAACCCGGAGCU | 1294 |
| gRNA262 | AGGGGUUACAGAGGAGACUU | 1295 |
| gRNA263 | AUCCCCGCAACGACUCUGCG | 1296 |
| gRNA264 | CAAGCCAAGCUCCGGGUUCC | 1297 |
| gRNA265 | AGUCUCCUCUGUAACCCCUA | 1298 |
| gRNA266 | UCCCCGCAACGACUCUGCGC | 1299 |
| gRNA267 | GGCACAGCAAGCCAAGCUCC | 1300 |
| gRNA268 | UCCUCUGUAACCCCUAAGGU | 1301 |
| gRNA269 | AACGACUCUGCGCGGGAACC | 1302 |
| gRNA270 | GGGCACAGCAAGCCAAGCUC | 1303 |
| gRNA271 | CCCGACCUUAGGGGUUACAG | 1304 |
| gRNA272 | UCUGCGCGGGAACCAGGAGC | 1305 |
| gRNA273 | GCUUGCUGUGCCCAGAGCUC | 1306 |
| gRNA274 | CCUCUGUAACCCCUAAGGUC | 1307 |
| gRNA275 | CUGCGCGGGAACCAGGAGCC | 1308 |
| gRNA276 | GCUGGAAACGAGGUGUUUCC | 1309 |
| gRNA277 | GGAACCAGGAGCCGGGAACC | 1310 |
| gRNA278 | UUGCGGGGAUGCUGGAAACG | 1311 |
| gRNA279 | AGCUCCGGGUUCCCGGCUCC | 1312 |

Any tracr sequence known in the art is contemplated for a gRNA described herein. In some embodiments, a gRNA described herein has a tracr sequence shown in Table 4 below, or a tracr sequence at least 70%, 75%, 80%, 85%, 90%, 95%, 96%, 97%, 98%, or 99% identical to the tracr sequence shown below (SEQ: SEQ ID NO).

TABLE 4
Exemplary TRACR Sequences

| SEQ | Sequence (5′ to 3′) |
|---|---|
| 653 | GUUUAAGAGCUAUGCUGGAAACAGCAUAGCAAGUUUAAAUAAGGCUAGUCCGUUAUCAACU |
| | UGAAAAAGUGGCACCGAGUCGGUGCUUUUUUU |
| 654 | GUUUUAGAGCUAGAAAUAGCAAGUUAAAAUAAGGCUAGUCCGUUAUCAACUUGAAAAAGUG |
| | GCACCGAGUCGGUGCUUUU |
| 655 | GUUUAAGAGCUAAGCUGGAAACAGCAUAGCAAGUUUAAAUAAGGCUAGUCCGUUAUCAACU |
| | UGAAAAAGUGGCACCGAGUCGGUGCUUUUUU |
| 656 | GUUUAAGAGCUAAGCUGGAAACAGCAUAGCAAGUUUAAAUAAGGCUAGUCCGUUAUCAACU |
| | UGAAAAAGUGGCACCGAGUCGGUGCUUUUUUU |

In some embodiments, the gRNA herein is provided to the cell directly (e.g., through an RNP complex together with the CRISPR-associated protein domain). In some embodiments, the gRNA is provided to the cell through an expression vector (e.g., a plasmid vector or a viral vector) introduced into the cell, where the cell then expresses the gRNA from the expression vector. Methods of introducing gRNAs and expression vectors into cells are well known in the art.

### III. Effector Domains

Epigenetic editors described herein include one or more effector protein domains (also “epigenetic effector domains,” or “effector domains,” as used herein) that effect epigenetic modification of a target gene. An epigenetic editor with one or more effector domains may modulate expression of a target gene without altering its nucleobase sequence. In some embodiments, an effector domain described herein may provide repression or silencing of expression of a target gene such as CIITA, e.g., by repressing transcription or by modifying or remodeling chromatin. Such effector domains are also referred to herein as “repression domains,” “repressor domains,” or “epigenetic repressor domains.” Non-limiting examples of chemical modifications that may be mediated by effector domains include methylation, demethylation, acetylation, deacetylation, phosphorylation, SUMOylation and/or ubiquitination of DNA or histone residues.

In some embodiments, an effector domain of an epigenetic editor described herein may make histone tail modifications, e.g., by adding or removing active marks on histone tails.

In some embodiments, an effector domain of an epigenetic editor described herein may comprise or recruit a transcription-related protein, e.g., a transcription repressor. The transcription-related protein may be endogenous or exogenous.

In some embodiments, an effector domain of an epigenetic editor described herein may, for example, comprise a protein that directly or indirectly blocks access of a transcription factor to the gene of interest harboring the target sequence.

An effector domain may be a full-length protein or a fragment thereof that retains the epigenetic effector function (a “functional domain”). Functional domains that are capable of modulating (e.g., repressing) gene expression can be derived from a larger protein. For example, functional domains that can reduce target gene expression may be identified based on sequences of repressor proteins. Amino acid sequences of gene expression-modulating proteins may be obtained from available genome browsers, such as the UCSD genome browser or Ensembl genome browser. Protein annotation databases such as UniProt or Pfam can be used to identify functional domains within the full protein sequence. As a starting point, the largest sequence, encompassing all regions identified by different databases, may be tested for gene expression modulation activity. Various truncations then may be tested to identify the minimal functional unit.

Variants of effector domains described herein are also contemplated by the present disclosure. A variant may, for example, refer to a polypeptide with at least about 85%, 90%, 91%, 92%, 93%, 94%, 95%, 96%, 97%, 98%, 99%, or 100% sequence identity and/or sequence similarity to a wildtype effector domain described herein. In particular embodiments, the variant retains at least about 85%, 90%, 91%, 92%, 93%, 94%, 95%, 96%, 97%, 98%, 99%, or 100% of the epigenetic effector function of the wildtype effector domain.

In some embodiments, an effector domain described herein may comprise a fusion of two or more effector domains (e.g., KOX1 KRAB and ZIM3). The effector domain may, for example, comprise a fusion of 2, 3, 4, 5, 6, 7, 8, 9, or 10 effector domains, such as effector domains described herein. In certain embodiments, an effector domain comprises a fusion of a truncated form of an effector domain and a second effector domain. In certain embodiments, an effector domain comprises a fusion of the truncated forms of two effector domains (e.g., fusions of the N- and C-terminal portions of the two effector domains).

In some embodiments, an epigenetic editor described herein may comprise 1 effector domain, 2 effector domains, 3 effector domains, 4 effector domains, 5 effector domains, 6 effector domains, 7 effector domains, 8 effector domains, 9 effector domains, 10 effector domains, or more. In certain embodiments, the epigenetic editor comprises one or more fusion proteins (e.g., one, two, or three fusion proteins), each with one or more effector domains (e.g., one, two, or three effector domains) linked to a DNA-binding domain. In some embodiments, the effector domains may induce a combination of epigenetic modifications, e.g., transcription repression and DNA methylation, DNA methylation and histone deacetylation, DNA methylation and histone demethylation, DNA methylation and histone methylation, DNA methylation and histone phosphorylation, DNA methylation and histone ubiquitylation, DNA methylation, and histone SUMOylation.

In certain embodiments, an effector domain described herein (e.g., DNMT3A and/or DNMT3L) is encoded by a nucleotide sequence as found in the native genome (e.g., human or murine) for that effector domain. In other embodiments, an effector domain described herein is encoded by a nucleotide sequence that has been codon-optimized for optimal expression in human cells.

Effector domains described herein may include, for example, transcriptional repressors, DNA methyltransferases, and/or histone modifiers, as further detailed below.

### A. Transcriptional Repressors

In some embodiments, an epigenetic effector domain described herein mediates repression of a target gene's expression (e.g., transcription). The effector domain may comprise, e.g., a Krüppel-associated box (KRAB) repressor domain, a Repressor Element Silencing Transcription Factor (REST) repressor domain, a KRAB-associated protein 1 (KAP1) domain, a MAD domain, a FKHR (forkhead in rhabdosarcoma gene) repressor domain, an EGR-1 (early growth response gene product-1) repressor domain, an ets2 repressor factor repressor domain (ERD), a MAD smSIN3 interaction domain (SID), a WRPW motif of the hairy-related basic helix-loop-helix (bHLH) repressor proteins, an HP1 alpha chromo-shadow repressor domain, an HP1 beta repressor domain, or any combination thereof. The effector domain may recruit one or more protein domains that repress expression of the target gene, e.g., through a scaffold protein. In some embodiments, the effector domain may recruit or interact with a scaffold protein domain that recruits a PRMT protein, a HDAC protein, a SETDB1 protein, or a NuRD protein domain.

In some embodiments, the effector domain comprises a functional domain derived from a zinc finger repressor protein, such as a KRAB domain. KRAB domains are found in approximately 400 human ZFP-based transcription factors. Descriptions of KRAB domains may be found, for example, in Ecco et al., Development (2017) 144 (15): 2719-29 and Lambert et al., Cell (2018) 172:650-65.

In certain embodiments, the effector domain comprises a repressor domain (e.g., KRAB) derived from KOX1/ZNF10, KOX8/ZNF708, ZNF43, ZNF184, ZNF91, HPF4, HTF10, or HTF34. In some embodiments, the effector domain comprises a repressor domain (e.g., KRAB) derived from ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37, ZNF34, ZNF250, ZNF547, ZNF273, ZNF354, ZFP82, ZNF224, ZNF33, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, or any combination thereof. For example, the repressor domain may be a KRAB domain derived from KOX1, ZIM3, ZFP28, or ZN627. In particular embodiments, the repressor domain is a ZIM3 KRAB domain. In further embodiments, the effector domain is derived from a human protein, e.g., a human ZIM3, a human KOX1, a human ZFP28, or a human ZN627.

Sequences of exemplary effector domains that may reduce or silence target gene expression, or protein sequences that contain them, are provided in Table 5 below (SEQ: SEQ ID NO). Further examples of repressors and transcriptional repressor domains can be found, e.g., in PCT Patent Publication WO 2021/226077 and Tycko et al., Cell (2020) 183 (7): 2020-35, each of which is incorporated herein by reference in its entirety.

TABLE 5
Exemplary Effector Domains That May
Reduce or Silence Gene Expression

| Protein | SEQ |
|---|---|
| ZIM3 | 33 |
| ZNF436 | 34 |
| ZNF257 | 35 |
| ZNF675 | 36 |
| ZNF490 | 37 |
| ZNF320 | 38 |
| ZNF331 | 39 |
| ZNF816 | 40 |
| ZNF680 | 41 |
| ZNF41 | 42 |
| ZNF189 | 43 |
| ZNF528 | 44 |
| ZNF543 | 45 |
| ZNF554 | 46 |
| ZNF140 | 47 |
| ZNF610 | 48 |
| ZNF264 | 49 |
| ZNF350 | 50 |
| ZNF8 | 51 |
| ZNF582 | 52 |
| ZNF30 | 53 |
| ZNF324 | 54 |
| ZNF98 | 55 |
| ZNF669 | 56 |
| ZNF677 | 57 |
| ZNF596 | 58 |
| ZNF214 | 59 |
| ZNF37A | 60 |
| ZNF34 | 61 |
| ZNF250 | 62 |
| ZNF547 | 63 |
| ZNF273 | 64 |
| ZNF354A | 65 |
| ZFP82 | 66 |
| ZNF224 | 67 |
| ZNF33A | 68 |
| ZNF45 | 69 |
| ZNF175 | 70 |
| ZNF595 | 71 |
| ZNF184 | 72 |
| ZNF419 | 73 |
| ZFP28-1 | 74 |
| ZFP28-2 | 75 |
| ZNF18 | 76 |
| ZNF213 | 77 |
| ZNF394 | 78 |
| ZFP1 | 79 |
| ZFP14 | 80 |
| ZNF416 | 81 |
| ZNF557 | 82 |
| ZNF566 | 83 |
| ZNF729 | 84 |
| ZIM2 | 85 |
| ZNF254 | 86 |
| ZNF764 | 87 |
| ZNF785 | 88 |
| ZNF10 (KOX1) | 89 |
| CBX5 (chromoshadow domain) | 90 |
| RYBP (YAF2_RYBP component of PRC1) | 91 |
| YAF2 (YAF2_RYBP component of PRC1) | 92 |
| MGA (component of PRC1.6) | 93 |
| CBX1 (chromoshadow) | 94 |
| SCMH1 (SAM_1/SPM) | 95 |
| MPP8 (Chromodomain) | 96 |
| SUMO3 (Rad60-SLD) | 97 |
| HERC2 (Cyt-b5) | 98 |
| BIN1 (SH3_9) | 99 |
| PCGF2 (RING finger protein domain) | 100 |
| TOX (HMG box) | 101 |
| FOXA1 (HNF3A C-terminal domain) | 102 |
| FOXA2 (HNF3B C-terminal domain) | 103 |
| IRF2BP1 (IRF-2BP1_2 N-terminal domain) | 104 |
| IRF2BP2 (IRF-2BP1_2 N-terminal domain) | 105 |
| IRF2BPL IRF-2BP1_2 N-terminal domain) | 106 |
| HOXA13 (homeodomain) | 107 |
| HOXB13 (homeodomain) | 108 |
| HOXC13 (homeodomain) | 109 |
| HOXA11 (homeodomain) | 110 |
| HOXC11 (homeodomain) | 111 |
| HOXC10 (homeodomain) | 112 |
| HOXA10 (homeodomain) | 113 |
| HOXB9 (homeodomain) | 114 |
| HOXA9 (homeodomain) | 115 |
| ZFP28_HUMAN | 116 |
| ZN334_HUMAN | 117 |
| ZN568_HUMAN | 118 |
| ZN37A_HUMAN | 119 |
| ZN181_HUMAN | 120 |
| ZN510_HUMAN | 121 |
| ZN862_HUMAN | 122 |
| ZN140_HUMAN | 123 |
| ZN208_HUMAN | 124 |
| ZN248_HUMAN | 125 |
| ZN571_HUMAN | 126 |
| ZN699_HUMAN | 127 |
| ZN726_HUMAN | 128 |
| ZIK1_HUMAN | 129 |
| ZNF2_HUMAN | 130 |
| Z705F_HUMAN | 131 |
| ZNF14_HUMAN | 132 |
| ZN471_HUMAN | 133 |
| ZN624_HUMAN | 134 |
| ZNF84_HUMAN | 135 |
| ZNF7_HUMAN | 136 |
| ZN891_HUMAN | 137 |
| ZN337_HUMAN | 138 |
| Z705G_HUMAN | 139 |
| ZN529_HUMAN | 140 |
| ZN729_HUMAN | 141 |
| ZN419_HUMAN | 142 |
| Z705A_HUMAN | 143 |
| ZNF45_HUMAN | 144 |
| ZN302_HUMAN | 145 |
| ZN486_HUMAN | 146 |
| ZN621_HUMAN | 147 |
| ZN688_HUMAN | 148 |
| ZN33A_HUMAN | 149 |
| ZN554_HUMAN | 150 |
| ZN878_HUMAN | 151 |
| ZN772_HUMAN | 152 |
| ZN224_HUMAN | 153 |
| ZN184_HUMAN | 154 |
| ZN544_HUMAN | 155 |
| ZNF57_HUMAN | 156 |
| ZN283_HUMAN | 157 |
| ZN549_HUMAN | 158 |
| ZN211_HUMAN | 159 |
| ZN615_HUMAN | 160 |
| ZN253_HUMAN | 161 |
| ZN226_HUMAN | 162 |
| ZN730_HUMAN | 163 |
| Z585A_HUMAN | 164 |
| ZN732_HUMAN | 165 |
| ZN681_HUMAN | 166 |
| ZN667_HUMAN | 167 |
| ZN649_HUMAN | 168 |
| ZN470_HUMAN | 169 |
| ZN484_HUMAN | 170 |
| ZN431_HUMAN | 171 |
| ZN382_HUMAN | 172 |
| ZN254_HUMAN | 173 |
| ZN124_HUMAN | 174 |
| ZN607_HUMAN | 175 |
| ZN317_HUMAN | 176 |
| ZN620_HUMAN | 177 |
| ZN141_HUMAN | 178 |
| ZN584_HUMAN | 179 |
| ZN540_HUMAN | 180 |
| ZN75D_HUMAN | 181 |
| ZN555_HUMAN | 182 |
| ZN658_HUMAN | 183 |
| ZN684_HUMAN | 184 |
| RBAK_HUMAN | 185 |
| ZN829_HUMAN | 186 |
| ZN582_HUMAN | 187 |
| ZN112_HUMAN | 188 |
| ZN716_HUMAN | 189 |
| HKR1_HUMAN | 190 |
| ZN350_HUMAN | 191 |
| ZN480_HUMAN | 192 |
| ZN416_HUMAN | 193 |
| ZNF92_HUMAN | 194 |
| ZN100_HUMAN | 195 |
| ZN736_HUMAN | 196 |
| ZNF74_HUMAN | 197 |
| CBX1_HUMAN | 198 |
| ZN443_HUMAN | 199 |
| ZN195_HUMAN | 200 |
| ZN530_HUMAN | 201 |
| ZN782_HUMAN | 202 |
| ZN791_HUMAN | 203 |
| ZN331_HUMAN | 204 |
| Z354C_HUMAN | 205 |
| ZN157_HUMAN | 206 |
| ZN727_HUMAN | 207 |
| ZN550_HUMAN | 208 |
| ZN793_HUMAN | 209 |
| ZN235_HUMAN | 210 |
| ZNF8_HUMAN | 211 |
| ZN724_HUMAN | 212 |
| ZN573_HUMAN | 213 |
| ZN577_HUMAN | 214 |
| ZN789_HUMAN | 215 |
| ZN718_HUMAN | 216 |
| ZN300_HUMAN | 217 |
| ZN383_HUMAN | 218 |
| ZN429_HUMAN | 219 |
| ZN677_HUMAN | 220 |
| ZN850_HUMAN | 221 |
| ZN454_HUMAN | 222 |
| ZN257_HUMAN | 223 |
| ZN264_HUMAN | 224 |
| ZFP82_HUMAN | 225 |
| ZFP14_HUMAN | 226 |
| ZN485_HUMAN | 227 |
| ZN737_HUMAN | 228 |
| ZNF44_HUMAN | 229 |
| ZN596_HUMAN | 230 |
| ZN565_HUMAN | 231 |
| ZN543_HUMAN | 232 |
| ZFP69_HUMAN | 233 |
| SUMO1_HUMAN | 234 |
| ZNF12_HUMAN | 235 |
| ZN169_HUMAN | 236 |
| ZN433_HUMAN | 237 |
| SUMO3_HUMAN | 238 |
| ZNF98_HUMAN | 239 |
| ZN175_HUMAN | 240 |
| ZN347_HUMAN | 241 |
| ZNF25_HUMAN | 242 |
| ZN519_HUMAN | 243 |
| Z585B_HUMAN | 244 |
| ZIM3_HUMAN | 245 |
| ZN517_HUMAN | 246 |
| ZN846_HUMAN | 247 |
| ZN230_HUMAN | 248 |
| ZNF66_HUMAN | 249 |
| ZFP1_HUMAN | 250 |
| ZN713_HUMAN | 251 |
| ZN816_HUMAN | 252 |
| ZN426_HUMAN | 253 |
| ZN674_HUMAN | 254 |
| ZN627_HUMAN | 255 |
| ZNF20_HUMAN | 256 |
| Z587B_HUMAN | 257 |
| ZN316_HUMAN | 258 |
| ZN233_HUMAN | 259 |
| ZN611_HUMAN | 260 |
| ZN556_HUMAN | 261 |
| ZN234_HUMAN | 262 |
| ZN560_HUMAN | 263 |
| ZNF77_HUMAN | 264 |
| ZN682_HUMAN | 265 |
| ZN614_HUMAN | 266 |
| ZN785_HUMAN | 267 |
| ZN445_HUMAN | 268 |
| ZFP30_HUMAN | 269 |
| ZN225_HUMAN | 270 |
| ZN551_HUMAN | 271 |
| ZN610_HUMAN | 272 |
| ZN528_HUMAN | 273 |
| ZN284_HUMAN | 274 |
| ZN418_HUMAN | 275 |
| MPP8_HUMAN | 276 |
| ZN490_HUMAN | 277 |
| ZN805_HUMAN | 278 |
| Z780B_HUMAN | 279 |
| ZN763_HUMAN | 280 |
| ZN285_HUMAN | 281 |
| ZNF85_HUMAN | 282 |
| ZN223_HUMAN | 283 |
| ZNF90_HUMAN | 284 |
| ZN557_HUMAN | 285 |
| ZN425_HUMAN | 286 |
| ZN229_HUMAN | 287 |
| ZN606_HUMAN | 288 |
| ZN155_HUMAN | 289 |
| ZN222_HUMAN | 290 |
| ZN442_HUMAN | 291 |
| ZNF91_HUMAN | 292 |
| ZN135_HUMAN | 293 |
| ZN778_HUMAN | 294 |
| RYBP_HUMAN | 295 |
| ZN534_HUMAN | 296 |
| ZN586_HUMAN | 297 |
| ZN567_HUMAN | 298 |
| ZN440_HUMAN | 299 |
| ZN583_HUMAN | 300 |
| ZN441_HUMAN | 301 |
| ZNF43_HUMAN | 302 |
| CBX5_HUMAN | 303 |
| ZN589_HUMAN | 304 |
| ZNF10_HUMAN | 305 |
| ZN563_HUMAN | 306 |
| ZN561_HUMAN | 307 |
| ZN136_HUMAN | 308 |
| ZN630_HUMAN | 309 |
| ZN527_HUMAN | 310 |
| ZN333_HUMAN | 311 |
| Z324B_HUMAN | 312 |
| ZN786_HUMAN | 313 |
| ZN709_HUMAN | 314 |
| ZN792_HUMAN | 315 |
| ZN599_HUMAN | 316 |
| ZN613_HUMAN | 317 |
| ZF69B_HUMAN | 318 |
| ZN799_HUMAN | 319 |
| ZN569_HUMAN | 320 |
| ZN564_HUMAN | 321 |
| ZN546_HUMAN | 322 |
| ZFP92_HUMAN | 323 |
| YAF2_HUMAN | 324 |
| ZN723_HUMAN | 325 |
| ZNF34_HUMAN | 326 |
| ZN439_HUMAN | 327 |
| ZFP57_HUMAN | 328 |
| ZNF19_HUMAN | 329 |
| ZN404_HUMAN | 330 |
| ZN274_HUMAN | 331 |
| CBX3_HUMAN | 332 |
| ZNF30_HUMAN | 333 |
| ZN250_HUMAN | 334 |
| ZN570_HUMAN | 335 |
| ZN675_HUMAN | 336 |
| ZN695_HUMAN | 337 |
| ZN548_HUMAN | 338 |
| ZN132_HUMAN | 339 |
| ZN738_HUMAN | 340 |
| ZN420_HUMAN | 341 |
| ZN626_HUMAN | 342 |
| ZN559_HUMAN | 343 |
| ZN460_HUMAN | 344 |
| ZN268_HUMAN | 345 |
| ZN304_HUMAN | 346 |
| ZIM2_HUMAN | 347 |
| ZN605_HUMAN | 348 |
| ZN844_HUMAN | 349 |
| SUMO5_HUMAN | 350 |
| ZN101_HUMAN | 351 |
| ZN783_HUMAN | 352 |
| ZN417_HUMAN | 353 |
| ZN182_HUMAN | 354 |
| ZN823_HUMAN | 355 |
| ZN177_HUMAN | 356 |
| ZN197_HUMAN | 357 |
| ZN717_HUMAN | 358 |
| ZN669_HUMAN | 359 |
| ZN256_HUMAN | 360 |
| ZN251_HUMAN | 361 |
| CBX4_HUMAN | 362 |
| PCGF2_HUMAN | 363 |
| CDY2_HUMAN | 364 |
| CDYL2_HUMAN | 365 |
| HERC2_HUMAN | 366 |
| ZN562_HUMAN | 367 |
| ZN461_HUMAN | 368 |
| Z324A_HUMAN | 369 |
| ZN766_HUMAN | 370 |
| ID2_HUMAN | 371 |
| TOX_HUMAN | 372 |
| ZN274_HUMAN | 373 |
| SCMH1_HUMAN | 374 |
| ZN214_HUMAN | 375 |
| CBX7_HUMAN | 376 |
| ID1_HUMAN | 377 |
| CREM_HUMAN | 378 |
| SCX_HUMAN | 379 |
| ASCL1_HUMAN | 380 |
| ZN764_HUMAN | 381 |
| SCML2_HUMAN | 382 |
| TWST1_HUMAN | 383 |
| CREB1_HUMAN | 384 |
| TERF1_HUMAN | 385 |
| ID3_HUMAN | 386 |
| CBX8_HUMAN | 387 |
| CBX4_HUMAN | 388 |
| GSX1_HUMAN | 389 |
| NKX22_HUMAN | 390 |
| ATF1_HUMAN | 391 |
| TWST2_HUMAN | 392 |
| ZNF17_HUMAN | 393 |
| TOX3_HUMAN | 394 |
| TOX4_HUMAN | 395 |
| ZMYM3_HUMAN | 396 |
| I2BP1_HUMAN | 397 |
| RHXF1_HUMAN | 398 |
| SSX2_HUMAN | 399 |
| I2BPL_HUMAN | 400 |
| ZN680_HUMAN | 401 |
| CBX1_HUMAN | 402 |
| TRI68_HUMAN | 403 |
| HXA13_HUMAN | 404 |
| PHC3_HUMAN | 405 |
| TCF24_HUMAN | 406 |
| CBX3_HUMAN | 407 |
| HXB13_HUMAN | 408 |
| HEY1_HUMAN | 409 |
| PHC2_HUMAN | 410 |
| ZNF81_HUMAN | 411 |
| FIGLA_HUMAN | 412 |
| SAM11_HUMAN | 413 |
| KMT2B_HUMAN | 414 |
| HEY2_HUMAN | 415 |
| JDP2_HUMAN | 416 |
| HXC13_HUMAN | 417 |
| ASCL4_HUMAN | 418 |
| HHEX_HUMAN | 419 |
| HERC2_HUMAN | 420 |
| GSX2_HUMAN | 421 |
| BIN1_HUMAN | 422 |
| ETV7_HUMAN | 423 |
| ASCL3_HUMAN | 424 |
| PHC1_HUMAN | 425 |
| OTP_HUMAN | 426 |
| I2BP2_HUMAN | 427 |
| VGLL2_HUMAN | 428 |
| HXA11_HUMAN | 429 |
| PDLI4_HUMAN | 430 |
| ASCL2_HUMAN | 431 |
| CDX4_HUMAN | 432 |
| ZN860_HUMAN | 433 |
| LMBL4_HUMAN | 434 |
| PDIP3_HUMAN | 435 |
| NKX25_HUMAN | 436 |
| CEBPB_HUMAN | 437 |
| ISL1_HUMAN | 438 |
| CDX2_HUMAN | 439 |
| PROP1_HUMAN | 440 |
| SIN3B_HUMAN | 441 |
| SMBT1_HUMAN | 442 |
| HXC11_HUMAN | 443 |
| HXC10_HUMAN | 444 |
| PRS6A_HUMAN | 445 |
| VSX1_HUMAN | 446 |
| NKX23_HUMAN | 447 |
| MTG16_HUMAN | 448 |
| HMX3_HUMAN | 449 |
| HMX1_HUMAN | 450 |
| KIF22_HUMAN | 451 |
| CSTF2_HUMAN | 452 |
| CEBPE_HUMAN | 453 |
| DLX2_HUMAN | 454 |
| ZMYM3_HUMAN | 455 |
| PPARG_HUMAN | 456 |
| PRIC1_HUMAN | 457 |
| UNC4_HUMAN | 458 |
| BARX2_HUMAN | 459 |
| ALX3_HUMAN | 460 |
| TCF15_HUMAN | 461 |
| TERA_HUMAN | 462 |
| VSX2_HUMAN | 463 |
| HXD12_HUMAN | 464 |
| CDX1_HUMAN | 465 |
| TCF23_HUMAN | 466 |
| ALX1_HUMAN | 467 |
| HXA10_HUMAN | 468 |
| RX HUMAN | 469 |
| CXXC5_HUMAN | 470 |
| SCML1_HUMAN | 471 |
| NFIL3_HUMAN | 472 |
| DLX6_HUMAN | 473 |
| MTG8_HUMAN | 474 |
| CBX8_HUMAN | 475 |
| CEBPD_HUMAN | 476 |
| SEC13_HUMAN | 477 |
| FIP1_HUMAN | 478 |
| ALX4_HUMAN | 479 |
| LHX3_HUMAN | 480 |
| PRIC2_HUMAN | 481 |
| MAGI3_HUMAN | 482 |
| NELL1_HUMAN | 483 |
| PRRX1_HUMAN | 484 |
| MTG8R_HUMAN | 485 |
| RAX2_HUMAN | 486 |
| DLX3_HUMAN | 487 |
| DLX1_HUMAN | 488 |
| NKX26_HUMAN | 489 |
| NAB1_HUMAN | 490 |
| SAMD7_HUMAN | 491 |
| PITX3_HUMAN | 492 |
| WDR5_HUMAN | 493 |
| MEOX2_HUMAN | 494 |
| NAB2_HUMAN | 495 |
| DHX8_HUMAN | 496 |
| FOXA2_HUMAN | 497 |
| CBX6_HUMAN | 498 |
| EMX2_HUMAN | 499 |
| CPSF6_HUMAN | 500 |
| HXC12_HUMAN | 501 |
| KDM4B_HUMAN | 502 |
| LMBL3_HUMAN | 503 |
| PHX2A_HUMAN | 504 |
| EMX1_HUMAN | 505 |
| NC2B_HUMAN | 506 |
| DLX4_HUMAN | 507 |
| SRY_HUMAN | 508 |
| ZN777_HUMAN | 509 |
| NELL1_HUMAN | 510 |
| ZN398_HUMAN | 511 |
| GATA3_HUMAN | 512 |
| BSH_HUMAN | 513 |
| SF3B4_HUMAN | 514 |
| TEAD1_HUMAN | 515 |
| TEAD3_HUMAN | 516 |
| RGAP1_HUMAN | 517 |
| PHF1_HUMAN | 518 |
| FOXA1_HUMAN | 519 |
| GATA2_HUMAN | 520 |
| FOXO3_HUMAN | 521 |
| ZN212_HUMAN | 522 |
| IRX4_HUMAN | 523 |
| ZBED6_HUMAN | 524 |
| LHX4_HUMAN | 525 |
| SIN3A_HUMAN | 526 |
| RBBP7_HUMAN | 527 |
| NKX61_HUMAN | 528 |
| TRI68_HUMAN | 529 |
| R51A1_HUMAN | 530 |
| MB3L1_HUMAN | 531 |
| DLX5_HUMAN | 532 |
| NOTC1_HUMAN | 533 |
| TERF2_HUMAN | 534 |
| ZN282_HUMAN | 535 |
| RGS12_HUMAN | 536 |
| ZN840_HUMAN | 537 |
| SPI2B_HUMAN_1 | 538 |
| PAX7_HUMAN | 539 |
| NKX62_HUMAN | 540 |
| ASXL2_HUMAN | 541 |
| FOXO1_HUMAN | 542 |
| GATA3_HUMAN | 543 |
| GATA1_HUMAN | 544 |
| ZMYM5_HUMAN | 545 |
| ZN783_HUMAN | 546 |
| SPI2B_HUMAN_2 | 547 |
| LRP1_HUMAN | 548 |
| MIXL1_HUMAN | 549 |
| SGT1_HUMAN | 550 |
| LMCD1_HUMAN | 551 |
| CEBPA_HUMAN | 552 |
| GATA2_HUMAN | 553 |
| SOX14_HUMAN | 554 |
| WTIP_HUMAN | 555 |
| PRP19_HUMAN | 556 |
| CBX6_HUMAN | 557 |
| NKX11_HUMAN | 558 |
| RBBP4_HUMAN | 559 |
| DMRT2_HUMAN | 560 |
| SMCA2_HUMAN | 561 |
| ZNF10_HUMAN | 562 |
| EED HUMAN | 563 |
| RCOR1_HUMAN | 564 |

A functional analog of any one of the above-listed proteins, i.e., a molecule having the same or substantially the same biological function (e.g., retaining 70% or more, 80% or more, 90% or more, 95% or more, or 98% or more) of the protein's transcription factor function) is encompassed by the present disclosure. For example, the functional analog may be an isoform or a variant of the above-listed protein, e.g., containing a portion of the above protein with or without additional amino acid residues and/or containing mutations relative to the above protein. In some embodiments, the functional analog has a sequence identity that is at least 75, 80, 85, 90, 95, 98, or 99% to one of the sequences listed in Table 5. Homologs, orthologs, and mutants of the above-listed proteins are also contemplated.

In certain embodiments, an epigenetic editor described herein comprises a KRAB domain derived from KOX1, ZIM3, ZFP28, or ZN627, and/or an effector domain derived from KAP1, MECP2, HP1a, HP1b, CBX8, CDYL2, TOX, TOX3, TOX4, EED, EZH2, RBBP4, RCOR1, or SCML2, optionally wherein the parental protein is a human protein. In particular embodiments, an epigenetic editor described herein comprises a domain derived from KOX1, ZIM3, ZFP28, and/or ZN627, optionally wherein the parental protein is a human protein. In certain embodiments, the epigenetic editor may comprise a KRAB domain derived from KOX1 (ZNF10), e.g., a human KOX1. In certain embodiments, the epigenetic editor may comprise a KRAB domain derived from ZIM3 (ZNF657 or ZNF264), e.g., a human ZIM3. In certain embodiments, the epigenetic editor may comprise a KRAB domain derived from ZFP28, e.g., a human ZFP28. In certain embodiments, the epigenetic editor may comprise a KRAB domain derived from ZN627, e.g., a human ZN627. In certain embodiments, an epigenetic editor described herein may comprise a CDYL2, e.g., a human CDYL2, and/or a TOX domain (e.g., a human TOX domain) in combination with a KOX1 KRAB domain (e.g., a human KOX1 KRAB domain).

In certain embodiments, an epigenetic effector described herein comprises a repressor domain derived from KOX1/ZNF10 (SEQ ID NO: 89). For example, the repressor domain may comprise the sequence of SEQ ID NO: 89, or a sequence at least 75%, 80%, 85%, 90%, 91%, 92%, 93%, 94%, 95%, 96%, 97%, 98%, or 99% identical to SEQ ID NO: 89.

In certain embodiments, an epigenetic effector described herein comprises a repressor domain derived from KOX1/ZNF10, as shown in Table 6 below:

TABLE 6
Exemplary Effector Domains Derived from KOX1/ZNF10

| Protein | Protein Sequence |
|---|---|
| KOX1/ZNF10 KRAB 1 | SEQ ID NO: 565 |
| KOX1/ZNF10 KRAB 2 | SEQ ID NO: 566 |
| KOX1/ZNF10 KRAB 3 | SEQ ID NO: 567 |
| KOX1/ZNF10 (aa 11-72) | SEQ ID NO: 568 |
| KOX1/ZNF10 (aa 11-108) | SEQ ID NO: 569 |
| KOX1/ZNF10 variant | SEQ ID NO: 570 |
| KOX1 KRAB-ZIM3 chimera | SEQ ID NO: 571 |
| ZIM3-KOX1 KRAB chimera | SEQ ID NO: 572 |

In particular embodiments, the repressor domain may comprise the amino acid sequence of SEQ ID NO: 565, or a sequence at least 75%, 80%, 85%, 90%, 91%, 92%, 93%, 94%, 95%, 96%, 97%, 98%, or 99% identical to SEQ ID NO: 565.

In particular embodiments, the repressor domain may comprise the amino acid sequence of SEQ ID NO: 566, or a sequence at least 75%, 80%, 85%, 90%, 91%, 92%, 93%, 94%, 95%, 96%, 97%, 98%, or 99% identical to SEQ ID NO: 566.

In particular embodiments, the repressor domain may comprise the amino acid sequence of SEQ ID NO: 567, or a sequence at least 75%, 80%, 85%, 90%, 91%, 92%, 93%, 94%, 95%, 96%, 97%, 98%, or 99% identical to SEQ ID NO: 567.

In particular embodiments, the repressor domain may comprise the amino acid sequence of SEQ ID NO: 568, or a sequence at least 75%, 80%, 85%, 90%, 91%, 92%, 93%, 94%, 95%, 96%, 97%, 98%, or 99% identical to SEQ ID NO: 568.

In particular embodiments, the repressor domain may comprise the amino acid sequence of SEQ ID NO: 569, or a sequence at least 75%, 80%, 85%, 90%, 91%, 92%, 93%, 94%, 95%, 96%, 97%, 98%, or 99% identical to SEQ ID NO: 569.

In particular embodiments, the repressor domain may comprise the amino acid sequence of SEQ ID NO: 570, or a sequence at least 75%, 80%, 85%, 90%, 91%, 92%, 93%, 94%, 95%, 96%, 97%, 98%, or 99% identical to SEQ ID NO: 570.

In particular embodiments, the repressor domain may comprise the amino acid sequence of SEQ ID NO: 571, or a sequence at least 75%, 80%, 85%, 90%, 91%, 92%, 93%, 94%, 95%, 96%, 97%, 98%, or 99% identical to SEQ ID NO: 571.

In particular embodiments, the repressor domain may comprise the amino acid sequence of SEQ ID NO: 572, or a sequence at least 75%, 80%, 85%, 90%, 91%, 92%, 93%, 94%, 95%, 96%, 97%, 98%, or 99% identical to SEQ ID NO: 572.

### B. DNA Methyltransferases

In some embodiments, an effector domain of an epigenetic editor described herein alters target gene expression through DNA modification, such as methylation. Highly methylated areas of DNA tend to be less transcriptionally active than less methylated areas. DNA methylation occurs primarily at CpG sites (shorthand for “C-phosphate-G-” or “cytosine-phosphate-guanine” sites). Many mammalian genes have promoter regions near or including CpG islands (nucleic acid regions with a high frequency of CpG dinucleotides).

An effector domain described herein may be, e.g., a DNA methyltransferase (DNMT) or a catalytic domain thereof, or may be capable of recruiting a DNA methyltransferase. DNMTs encompass enzymes that catalyze the transfer of a methyl group to a DNA nucleotide, such as canonical cytosine-5 DNMTs that catalyze the addition of methyl groups to genomic DNA (e.g., DNMT1, DNMT3A, DNMT3B, and DNMT3C). This term also encompasses non-canonical family members that do not catalyze methylation themselves but that recruit (including activate) catalytically active DNMTs; a non-limiting example of such a DNMT is DNMT3L. See, e.g., Lyko, Nat Review (2018) 19:81-92. Unless otherwise indicated, a DNMT domain may refer to a polypeptide domain derived from a catalytically active DNMT (e.g., DNMT1, DNMT3A, and DNMT3B) or from a catalytically inactive DNMT (e.g., DNMT3L). A DNMT may repress expression of the target gene through the recruitment of repressive regulatory proteins. In some embodiments, the methylation is at a CG (or CpG) dinucleotide sequence. In some embodiments, the methylation is at a CHG or CHH sequence, where H is any one of A, T, or C.

In some embodiments, a DNMT described herein can be an animal DNMT (e.g., a mammalian DNMT), a plant DNMT, a fungal DNMT, or a bacterial DNMT. A bacterial DNMT can be obtained from a bacterial species (e.g., a coccus bacterium, bacillus bacterium, spiral bacterium, or an intracellular, gram-positive, or gram-negative bacterium. In certain embodiments, the bacterial species is Mycoplasmatales bacterium, Mycoplasma marinum, or Spiroplasma chinense. In certain embodiments, the bacterial species is not M. penetrans, S. monbiae, H. parainfluenzae, A. luteus, H. aegyptius, H. haemolyticus, Moraxella, E. coli, T. aquaticus, C. crescentus, or C. difficile. In certain embodiments, an epigenetic editor described herein comprises a DNMT domain comprising SEQ ID NO: 601, or a sequence at least 75%, 80%, 85%, 90%, 91%, 92%, 93%, 94%, 95%, 96%, 97%, 98%, or 99% identical to SEQ ID NO: 601. In certain embodiments, an epigenetic editor described herein comprises a DNMT domain comprising SEQ ID NO: 602, or a sequence at least 75%, 80%, 85%, 90%, 91%, 92%, 93%, 94%, 95%, 96%, 97%, 98%, or 99% identical to SEQ ID NO: 602. In certain embodiments, an epigenetic editor described herein comprises a DNMT domain comprising SEQ ID NO: 603, or a sequence at least 75%, 80%, 85%, 90%, 91%, 92%, 93%, 94%, 95%, 96%, 97%, 98%, or 99% identical to SEQ ID NO: 603.

In certain embodiments, DNMTs in the epigenetic editors described herein may include, e.g., DNMT1, DNMT3A, DNMT3B, and/or DNMT3C. In some embodiments, the DNMT is a mammalian (e.g., human or murine) DNMT. In particular embodiments, the DNMT is DNMT3A (e.g., human DNMT3A). In certain embodiments, an epigenetic editor described herein comprises a DNMT3A domain comprising SEQ ID NO: 574, or a sequence at least 75%, 80%, 85%, 90%, 91%, 92%, 93%, 94%, 95%, 96%, 97%, 98%, or 99% identical to SEQ ID NO: 574. In certain embodiments, an epigenetic editor described herein comprises a DNMT3A domain comprising SEQ ID NO: 575, or a sequence at least 75%, 80%, 85%, 90%, 91%, 92%, 93%, 94%, 95%, 96%, 97%, 98%, or 99% identical to SEQ ID NO: 575. In some embodiments, the DNMT3A domain may have, e.g., a mutation at position H739 (such as H739A or H739E), R771 (such as R771L) and/or R836 (such as R836A or R836Q), or any combination thereof (numbering according to SEQ ID NO: 574).

In some embodiments, an effector domain described herein may be a DNMT-like domain. As used herein a “DNMT-like domain” is a regulatory factor of DNMT that may activate or recruit other DNMT domains, but does not itself possess methylation activity. In some embodiments, the DNMT-like domain is a mammalian (e.g., human or mouse) DNMT-like domain. In certain embodiments, the DNMT-like domain is DNMT3L, which may be, for example, human DNMT3L or mouse DNMT3L. In certain embodiments, an epigenetic editor described herein comprises a DNMT3L domain comprising SEQ ID NO: 578, or a sequence at least 75%, 80%, 85%, 90%, 91%, 92%, 93%, 94%, 95%, 96%, 97%, 98%, or 99% identical to SEQ ID NO: 578. In certain embodiments, an epigenetic editor herein comprises a DNMT3L domain comprising SEQ ID NO: 579, or a sequence at least 75%, 80%, 85%, 90%, 91%, 92%, 93%, 94%, 95%, 96%, 97%, 98%, or 99% identical to SEQ ID NO: 579. In certain embodiments, an epigenetic editor described herein comprises a DNMT3L domain comprising SEQ ID NO: 580, or a sequence at least 75%, 80%, 85%, 90%, 91%, 92%, 93%, 94%, 95%, 96%, 97%, 98%, or 99% identical to SEQ ID NO: 580. In certain embodiments, an epigenetic editor described herein comprises a DNMT3L domain comprising SEQ ID NO: 581, or a sequence at least 75%, 80%, 85%, 90%, 91%, 92%, 93%, 94%, 95%, 96%, 97%, 98%, or 99% identical to SEQ ID NO: 581. In some embodiments, the DNMT3L domain may have, e.g., a mutation corresponding to that at position D226 (such as D226V), Q268 (such as Q268K), or both (numbering according to SEQ ID NO: 578).

In certain embodiments, an epigenetic editor herein may comprise comprising both DNMT and DNMT-like effector domains. For example, the epigenetic editor may comprise a DNMT3A-3L domain, wherein DNMT3A and DNMT3L may be covalently linked. In other embodiments, an epigenetic editor described herein may comprise an effector domain that comprises only a DNMT3A domain (e.g., human DNMT3A), or only a DNMT-like domain (e.g., DNMT3L, which may be human or mouse DNMT3L).

Table 7 below provides exemplary DNMTs that may be part of an epigenetic effector domain described herein, or from which an effector domain of an epigenetic editor described herein may be derived.

TABLE 7
Exemplary DNMT Sequences

| Protein Name | Species | Target | Protein Sequence |
|---|---|---|---|
| DNMT1 | Human | 5mC | SEQ ID NO: 573 |
| DNMT3A (h3A) | Human | 5mC | SEQ ID NO: 574 |
| DNMT3A | Human | 5mC | SEQ ID NO: 575 |
| (catalytic domain) | | | |
| (h3As) | | | |
| DNMT3B | Human | 5mC | SEQ ID NO: 576 |
| DNMT3C | Mouse | 5mC | SEQ ID NO: 577 |
| DNMT3L (h3L) | Human | 5mC | SEQ ID NO: 578 |
| DNMT3L (catalytic | Human | 5mC | SEQ ID NO: 579 |
| domain) (h3Ls) | | | |
| DNMT3L (m3L) | Mouse | 5mC | SEQ ID NO: 580 |
| DNMT3L (catalytic | Mouse | 5mC | SEQ ID NO: 581 |
| domain) (m3Ls) | | | |
| DNMT3L | Ailuropoda melanoleuca | 5mC | SEQ ID NO: 582 |
| DNMT3L (catalytic | Ailuropoda melanoleuca | 5mC | SEQ ID NO: 583 |
| domain) | | | |
| DNMT3L | Carlito syrichta | 5mC | SEQ ID NO: 584 |
| DNMT3L (catalytic | Carlito syrichta | 5mC | SEQ ID NO: 585 |
| domain) | | | |
| DNMT3L | Meriones unguiculatus | 5mC | SEQ ID NO: 586 |
| DNMT3L (catalytic | Meriones unguiculatus | 5mC | SEQ ID NO: 587 |
| domain) | | | |
| DNMT3L | Ochotona princeps | 5mC | SEQ ID NO: 588 |
| DNMT3L (catalytic | Ochotona princeps | 5mC | SEQ ID NO: 589 |
| domain) | | | |
| DNMT3L | Neosciurus carolinensis | 5mC | SEQ ID NO: 590 |
| DNMT3L (catalytic | Neosciurus carolinensis | 5mC | SEQ ID NO: 591 |
| domain) | | | |
| DNMT3L | Bison bison | 5mC | SEQ ID NO: 592 |
| DNMT3L (catalytic | Bison bison | 5mC | SEQ ID NO: 593 |
| domain) | | | |
| DNMT3L | Equus przewalskii | 5mC | SEQ ID NO: 594 |
| DNMT3L (catalytic | Equus przewalskii | 5mC | SEQ ID NO: 595 |
| domain) | | | |
| DNMT3L | Mus caroli | 5mC | SEQ ID NO: 596 |
| DNMT3L (catalytic | Mus caroli | 5mC | SEQ ID NO: 597 |
| domain) | | | |
| DNMT3L | Pan troglodytes | 5mC | SEQ ID NO: 598 |
| DNMT3L (catalytic | Pan troglodytes | 5mC | SEQ ID NO: 599 |
| domain) | | | |
| TRDMT1 | Human | tRNA 5mC | SEQ ID NO: 600 |
| (DNMT2) | | | |
| DNA cytosine | Mycoplasmatales | 5mC | SEQ ID NO: 601 |
| methyltransferase | bacterium | | |
| DNA cytosine | Mycoplasma marinum | 5mC | SEQ ID NO: 602 |
| methyltransferase | | | |
| DNA (cytosine-5-)- | Spiroplasma chinense | 5mC | SEQ ID NO: 603 |
| methyltransferase | | | |
| M.Mpel | Mycoplasma penetrans | 5mC | SEQ ID NO: 604 |
| M.SssI | Spiroplasma monobiae | 5mC | SEQ ID NO: 605 |
| M.HpaII | Haemophilus parainfluenzae | 5mC (CCGG) | SEQ ID NO: 606 |
| M.Alul | Arthrobacter luteus | 5mC (AGCT) | SEQ ID NO: 607 |
| M.HaeIII | Haemophilus aegyptius | 5mC (GGCC) | SEQ ID NO: 608 |
| M.HhaI | Haemophilus haemolyticus | 5mC (GCGC) | SEQ ID NO: 609 |
| M.MspI | Moraxella | 5mC (CCGG) | SEQ ID NO: 610 |
| Masc 1 | Ascobolus | 5mC | SEQ ID NO: 611 |
| MET1 | Arabidopsis | 5mC | SEQ ID NO: 612 |
| Masc2 | Ascobolus | 5mC | SEQ ID NO: 613 |
| Dim-2 | Neurospora | 5mC | SEQ ID NO: 614 |
| dDnmt2 | Drosophila | 5mC | SEQ ID NO: 615 |
| Pmt1 | S. pombe | 5mC | SEQ ID NO: 616 |
| DRM1 | Arabidopsis | 5mC | SEQ ID NO: 617 |
| DRM2 | Arabidopsis | 5mC | SEQ ID NO: 618 |
| CMT1 | Arabidopsis | 5mC | SEQ ID NO: 619 |
| CMT2 | Arabidopsis | 5mC | SEQ ID NO: 620 |
| CMT3 | Arabidopsis | 5mC | SEQ ID NO: 621 |
| Rid | Neurospora | 5mC | SEQ ID NO: 622 |
| hsdM gene | bacteria (E. coli, strain 12) | m6A | SEQ ID NO: 623 |
| hsdS gene | bacteria (E. coli, strain 12) | m6A | SEQ ID NO: 624 |
| M.TaqI | Bacteria (Thermus aquaticus) | m6A | SEQ ID NO: 625 |
| M.EcoDam | E. coli | m6A | SEQ ID NO: 626 |
| M.CcrMI | Caulobacter crescentus | m6A | SEQ ID NO: 627 |
| CamA | Clostridioides difficile | m6A | SEQ ID NO: 628 |

A functional analog of any one of the above-listed proteins, i.e., a molecule having the same or substantially the same biological function (e.g., retaining 70% or more, 80% or more, 90% or more, 95% or more, or 98% or more) of the protein's DNA methylation function or recruiting function) is encompassed by the present disclosure. For example, the functional analog may be an isoform or a variant of the above-listed protein, e.g., containing a portion of the above protein with or without additional amino acid residues and/or containing mutations relative to the above protein. In some embodiments, the functional analog has a sequence identity that is at least 75, 80, 85, 90, 95, 98, or 99% to one of the sequences listed in Table 7. In some embodiments, the effector domain herein comprises only the functional domain (or functional analog thereof), e.g., the catalytic domain or recruiting domain, of an above-listed protein. In some embodiments, the effector domain herein comprises one or more epigenetic effector domains selected from Table 7, or functional homologs, orthologs, or variants thereof.

As used herein, a DNMT domain (e.g., a DNMT3A domain or a DNMT3L domain) refers to a protein domain that is identical to the parental protein (e.g., a human or murine DNMT3A or DNMT3L) or a functional analog thereof (e.g., having a functional fragment, such as a catalytic fragment or recruiting fragment, of the parental protein; and/or having mutations that improve the activity of the DNMT protein).

An epigenetic editor herein may effect methylation at, e.g., 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 or more CpG dinucleotide sequences in the target gene or chromosome. The CpG dinucleotide sequences may be located within or near the target gene in CpG islands, or may be located in a region that is not a CpG island. A CpG island generally refers to a nucleic acid sequence or chromosome region that comprises a high frequency of CpG dinucleotides. For example, a CpG island may comprise at least 50% GC content. The CpG island may have a high observed-to-expected CpG ratio, for example, an observed-to-expected CpG ratio of at least 60%. As used herein, an observed-to-expected CpG ratio is determined by Number of CpG*(sequence length)/(Number of C*Number of G). In some embodiments, the CpG island has an observed-to-expected CpG ratio of at least 60%, 70%, 80%, 90% or more. A CpG island may be a sequence or region of, e.g., at least 200, 250, 300, 350, 400, 450, 500, 550, 600, 650, 700, 750, or 800 nucleotides. In some embodiments, only 1, or less than 2, 3, 4, 5, 6, 7, 8, 9, 10, 11, 12, 13, 14, 15, 16, 17, 18, 19, 20, 30, 40, or 50 CpG dinucleotides are methylated by the epigenetic editor.

In some embodiments, an epigenetic editor herein effects methylation at a hypomethylated nucleic acid sequence, i.e., a sequence that may lack methyl groups on the 5-methyl cytosine nucleotides (e.g., in CpG) as compared to a standard control. Hypomethylation may occur, for example, in aging cells or in cancer (e.g., early stages of neoplasia) relative to a younger cell or non-cancer cell, respectively.

In some embodiments, an epigenetic editor described herein induces methylation at a hypermethylated nucleic acid sequence.

In some embodiments, methylation may be introduced by the epigenetic editor at a site other than a CpG dinucleotide. For example, the target gene sequence may be methylated at the C nucleotide of CpA, CpT, or CpC sequences. In some embodiments, an epigenetic editor comprises a DNMT3A domain and effects methylation at CpG, CpA, CpT, CpC sequences, or any combination thereof. In some embodiments, an epigenetic editor comprises a DNMT3A domain that lacks a regulatory subdomain and only maintains a catalytic domain. In some embodiments, the epigenetic editor comprising a DNMT3A catalytic domain effects methylation exclusively at CpG sequences. In some embodiments, an epigenetic editor comprising a DNMT3A domain that comprises a mutation, e.g. a R836A or R836Q mutation (numbering according to SEQ ID NO: 574), has higher methylation activity at CpA, CpC, and/or CpT sequences as compared to an epigenetic editor comprising a wildtype DNMT3A domain.

### C. Histone Modifiers

In some embodiments, an effector domain of an epigenetic editor herein mediates histone modification. Histone modifications play a structural and biochemical role in gene transcription, such as by formation or disruption of the nucleosome structure that binds to the histone and prevents gene transcription. Histone modifications may include, for example, acetylation, deacetylation, methylation, phosphorylation, ubiquitination, SUMOylation and the like, e.g., at their N-terminal ends (“histone tails”). These modifications maintain or specifically convert chromatin structure, thereby controlling responses such as gene expression, DNA replication, DNA repair, and the like, which occur on chromosomal DNA. Post-translational modification of histones is an epigenetic regulatory mechanism and is considered essential for the genetic regulation of eukaryotic cells. Recent studies have revealed that chromatin remodeling factors such as SWI/SNF, RSC, NURF, NRD, and the like, which facilitate transcription factor access to DNA by modifying the nucleosome structure; histone acetyltransferases (HATs) that regulate the acetylation state of histones; and histone deacetylases (HDACs), act as important regulators.

In particular, the unstructured N-termini of histones may be modified by acetylation, deacetylation, methylation, ubiquitylation, phosphorylation, SUMOylation, ribosylation, citrullination O-GlcNAcylation, crotonylation, or any combination thereof. For example, histone acetyltransferases (HATs) utilize acetyl-CoA as a cofactor and catalyze the transfer of an acetyl group to the epsilon amino group of the lysine side chains. This neutralizes the lysine's positive charge and weakens the interactions between histones and DNA, thus opening the chromosomes for transcription factors to bind and initiate transcription. Acetylation of K14 and K9 lysines of histone H3 by histone acetyltransferase enzymes may be linked to transcriptional competence in humans. Lysine acetylation may directly or indirectly create binding sites for chromatin-modifying enzymes that regulate transcriptional activation. On the other hand, histone methylation of lysine 9 of histone H3 may be associated with heterochromatin, or transcriptionally silent chromatin.

In certain embodiments, an effector domain of an epigenetic editor described herein comprises a histone methyltransferase domain. The effector domain may comprise, for example, a DOT1L domain, a SET domain, a SUV39H1 domain, a G9a/EHMT2 protein domain, an EZH1 domain, an EZH2 domain, a SETDB1 domain, or any combination thereof. In particular embodiments, the effector domain comprises a histone-lysine-N-methyltransferase SETDB1 domain.

In some embodiments, the effector domain comprises a histone deacetylase protein domain. In certain embodiments, the effector domain comprises a HDAC family protein domain, for example, a HDAC1, HDAC3, HDAC5, HDAC7, or HDAC9 protein domain. In particular embodiments, the effector domain comprises a nucleosome remodeling and deacetylase complex (NURD), which removes acetyl groups from histones.

### D. Other Effector Domains

In some embodiments, the effector domain comprises a tripartite motif containing protein (TRIM28, TIF1-beta, or KAP1). In certain embodiments, the effector domain comprises one or more KAP1 proteins. A KAP1 protein in an epigenetic editor herein may form a complex with one or more other effector domains of the epigenetic editor or one or more proteins involved in modulation of gene expression in a cellular environment. For example, KAP1 may be recruited by a KRAB domain of a transcriptional repressor. A KAP1 protein domain may interact with or recruit one or more protein complexes that reduces or silences gene expression. In some embodiments, KAP1 interacts with or recruits a histone deacetylase protein, a histone-lysine methyltransferase protein, a chromatin remodeling protein, and/or a heterochromatin protein. For example, a KAP1 protein domain may interact with or recruit a heterochromatin protein 1 (HP1) protein, a SETDB1 protein, an HDAC protein, and/or a NuRD protein complex component. In some embodiments, a KAP1 protein domain interacts with or recruits a ZFP90 protein (e.g., isoform 2 of ZFP90), and/or a FOXP3 protein. An exemplary KAP1 amino acid sequence is shown in SEQ ID NO: 629.

In some embodiments, the effector domain comprises a protein domain that interacts with or is recruited by one or more DNA epigenetic marks. For example, the effector domain may comprise a methyl CpG binding protein 2 (MECP2) protein that interacts with methylated DNA nucleotides in the target gene (which may or may not be at a CpG island of the target gene). An MECP2 protein domain in an epigenetic editor described herein may induce condensed chromatin structure, thereby reducing or silencing expression of the target gene. In some embodiments, an MECP2 protein domain in an epigenetic editor described herein may interact with a histone deacetylase (e.g. HDAC), thereby repressing or silencing expression of the target gene. In some embodiments, an MECP2 protein domain in an epigenetic editor described herein may block access of a transcription factor or transcriptional activator to the target sequence, thereby repressing or silencing expression of the target gene. An exemplary MECP2 amino acid sequence is shown in SEQ ID NO: 630.

Also contemplated as effector domains for the epigenetic editors described herein are, e.g., a chromoshadow domain, a ubiquitin-2 like Rad60 SUMO-like (Rad60-SLD/SUMO) domain, a chromatin organization modifier domain (Chromo) domain, a Yaf2/RYBP C-terminal binding motif domain (YAF2_RYBP), a CBX family C-terminal motif domain (CBX7_C), a zinc finger C3HC4 type (RING finger) domain (ZF-C3HC4_2), a cytochrome b5 domain (Cyt-b5), a helix-loop-helix domain (HLH), a helix-hairpin-helix motif domain (e.g., HHH_3), a high mobility group box domain (HMG-box), a basic leucine zipper domain (e.g., bZIP_1 or bZIP_2), a Myb_DNA-binding domain, a homeodomain, a MYM-type zinc finger with FCS sequence domain (ZF-FCS), an interferon regulatory factor 2-binding protein zinc finger domain (IRF-2BP1_2), an SSX repressor domain (SSXRD), a B-box-type zinc finger domain (ZF-B_box), a CXXC zinc finger domain (ZF-CXXC), a regulator of chromosome condensation 1 domain (RCC1), an SRC homology 3 domain (SH3_9), a sterile alpha motif domain (SAM_1), a sterile alpha motif domain (SAM_2), a sterile alpha motif/Pointed domain (SAM_PNT), a Vestigial/Tondu family domain (Vg_Tdu), a LIM domain, an RNA recognition motif domain (RRM_1), a paired amphipathic helix domain (PAH), a proteasomal ATPase OB C-terminal domain (Prot_ATP_ID_OB), a nervy homology 2 domain (NHR2), a hinge domain of cleavage stimulation factor subunit 2 (CSTF2_hinge), a PPAR gamma N-terminal region domain (PPARgamma_N), a CDC48 N-terminal domain (CDC48_2), a WD40 repeat domain (WD40), a Fip1 motif domain (Fip1), a PDZ domain (PDZ_6), a Von Willebrand factor type C domain (VWC), a NAB conserved region 1 domain (NCD1), an S1 RNA-binding domain (S1), an HNF3 C-terminal domain (HNF_C), a Tudor domain (Tudor_2), a histone-like transcription factor (CBF/NF-Y) and archaeal histone domain (CBFD_NFYB_HMF), a zinc finger protein domain (DUF3669), an EGF-like domain (cEGF), a GATA zinc finger domain (GATA), a TEA/ATTS domain (TEA), a phorbol esters/diacylglycerol binding domain (C1-1), polycomb-like MTF2 factor 2 domain (Mtf2_C), a transactivation domain of FOXO protein family (FOXO-TAD), a homeobox KN domain (Homeobox_KN), a BED zinc finger domain (ZF-BED), a zinc finger of C3HC4-type RING domain (ZF-C3HC4_4), a RAD51 interacting motif domain (RAD51_interact), a p55-binding region of a methyl-CpG-binding domain protein MBD (MBDa), a Notch domain, a Raf-like Ras-binding domain (RBD), a Spin/Ssty family domain (Spin-Ssty), a PHD finger domain (PHD_3), a Low-density lipoprotein receptor domain class A (Ldl_recept_a), a CS domain, a DM DNA-binding domain, and a QLQ domain.

In some embodiments, the effector domain is a protein domain comprising a YAF2_RYBP domain or homeodomain or any combination thereof. In certain embodiments, the homeodomain of the YAF2_RYBP domain is a PRD domain, an NKL domain, a HOXL domain, or a LIM domain. In particular embodiments, the YAF2_RYBP domain may comprise a 32 amino acid Yaf2/RYBP C-terminal binding motif domain (32 aa RYBP).

In some embodiments, the effector domain comprises a protein domain selected from a group consisting of SUMO3 domain, Chromo domain from M phase phosphoprotein 8 (MPP8), chromoshadow domain from Chromobox 1 (CBX1), and SAM_1/SPM domain from Scm Polycomb Group Protein Homolog 1 (SCMH1).

In some embodiments, the effector domain comprises an HNF3 C-terminal domain (HNF_C). The HNF_C domain may be from FOXA1 or FOXA2. In certain embodiments, the HNF_C domain comprises an EH1 (engrailed homology 1) motif.

In some embodiments, the effector domain may comprise an interferon regulatory factor 2-binding protein zinc finger domain (IRF-2BP1_2), a Cyt-b5 domain from DNA repair factor HERC2 E3 ligase, a variant SH3 domain (SH3_9) from Bridging Integrator 1 (BIN1), an HMG-box domain from transcription factor TOX or ZF-C3HC4_2 RING finger domain from the polycomb component PCGF2, a Chromodomain-helicase-DNA binding protein 3 (CHD3) domain, or a ZNF783 domain.

### IV. Epigenetic Editors

Provided herein are epigenetic editors (i.e., epigenetic editing systems) that direct epigenetic modification(s) to a target sequence in a gene of interest, e.g., using one or more DNA-binding domains as described herein and one or more effector domains (e.g., epigenetic repressor domains) as described herein, in any combination. The DNA-binding domain (in concert with a guide polynucleotide such as one described herein, where the DNA-binding domain is a polynucleotide guided DNA-binding domain) directs the effector domain to epigenetically modify the target sequence, resulting in gene repression or silencing that may be durable and inheritable across cell generations. In some aspects, the epigenetic editors described herein can repress or silence genes reversibly or irreversibly in cells.

In particular embodiments, an epigenetic editor described herein comprises one or more fusion proteins, each comprising (1) DNA-binding domain(s) and (2) effector domain(s). The effector domains may be on one or more fusion proteins comprised by the epigenetic editor. For example, a single fusion protein may comprise all of the effector domains with a DNA-binding domain. Alternatively, the effector domains or subsets thereof may be on separate fusion proteins, each with a DNA-binding domain (which may be the same or different). A fusion protein described herein may further comprise one or more linkers (e.g., peptide linkers), detectable tags, nuclear localization signals (NLSs), or any combination thereof. As used herein, a “fusion protein” refers to a chimeric protein in which two or more coding sequences (e.g., for DNA-binding domain(s) and/or effector domain(s)) are covalently or non-covalently joined, directly or indirectly.

In some embodiments, an epigenetic editor described herein comprises 2, 3, 4, 5, 6, 7, 8, 9, 10, or more effector (e.g., repression/repressor) domains, which may be identical or different. In certain embodiments, two or more of said effector domains function synergistically. Combinations of effector domains may comprise DNA methylation domains, histone deacetylation domains, histone methylation domains, and/or scaffold domains that recruit any of the above. For example, an epigenetic editor described herein may comprise one or more transcriptional repressor domains (e.g., a KRAB domain such as KOX1, ZIM3, ZFP28, or ZN627 KRAB) in combination with one or more DNA methylation domains (e.g., a DNMT domain) and/or recruiter domain (e.g., a DNMT3L domain). Such an epigenetic editor may comprise, for instance, a KRAB domain, a DNMT3A domain, and a DNMT3L domain. In some embodiments, the epigenetic editor further comprises an additional effector domain (e.g., a KAP1, MECP2, HP1b, CBX8, CDYL2, TOX, TOX3, TOX4, EED, RBBP4, RCOR1, or SCML2 domain). In some embodiments, the additional effector domain is a CDYL2, TOX, TOX3, TOX4, or HP1a domain. For example, an epigenetic editor described herein may comprise a CDYL2 and/or a TOX domain in combination with a KRAB domain (e.g., a KOX1 KRAB domain).

### A. Linkers

A fusion protein as described herein may comprise one or more linkers that connect components of the epigenetic editor. A linker may be a peptide or non-peptide linker.

In some embodiments, one or more linkers utilized in an epigenetic editor provided herein is a peptide linker, i.e., a linker comprising a peptide moiety. A peptide linker can be any length applicable to the epigenetic editor fusion proteins described herein. In some embodiments, the linker can comprise a peptide between 1 and 200 (e.g., between 1 and 80) amino acids. In some embodiments, the linker comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. Longer or shorter linkers are also contemplated. In some embodiments, the peptide linker is 3, 4, 5, 6, 7, 8, 9, 10, 11, 12, 13, 14, 15, 16, 17, 18, 19, 20, 25, 30, 25, 40, 45, 50, 55, 60, 65, 70, 75, 80, 85, 90, 95, or 100 amino acids in length. For example, the peptide linker may be 4, 5, 16, 20, 24, 27, 32, 40, 64, 92, or 104 amino acids in length. The peptide linker may be a flexible or rigid linker. In particular embodiments, the peptide linker comprises the amino acid sequence of any one of SEQ ID NOs: 631-637 and 664-666 or a sequence at least 75%, 80%, 85%, 90%, 91%, 92%, 93%, 94%, 95%, 96%, 97%, 98%, or 99% identical thereto.

In certain embodiments, the peptide linker is an XTEN linker. Such a linker may comprise part of the XTEN sequence (Schellenberger et al., Nat Biotechnol (2009) 27 (1): 1186-90), an unstructured hydrophilic polypeptide consisting only of residues G, S, P, T, E, and A. The term “XTEN” as used herein refers to a recombinant peptide or polypeptide lacking hydrophobic amino acid residues. XTEN linkers typically are unstructured and comprise a limited set of natural amino acids. Fusion of XTEN to proteins alters its hydrodynamic properties and reduces the rate of clearance and degradation of the fusion protein. These XTEN fusion proteins are produced using recombinant technology, without the need for chemical modifications, and degraded by natural pathways. The XTEN linker may be, for example, 5, 10, 16, 20, 26, or 80 amino acids in length. In some embodiments, the XTEN linker is 16 amino acids in length. In some embodiments, the XTEN linker is 80 amino acids in length. In certain embodiments, the XTEN linker may be XTEN10, XTEN16, XTEN20, or XTEN80. In certain embodiments, the XTEN linker may comprise the amino acid sequence of any one of SEQ ID NOs: 638-643 or a sequence at least 75%, 80%, 85%, 90%, 91%, 92%, 93%, 94%, 95%, 96%, 97%, 98%, or 99% identical thereto. In particular embodiments, the XTEN linker comprises the amino acid sequence of SEQ ID NO: 638. In particular embodiments, the XTEN linker comprises the amino acid sequence of SEQ ID NO: 643.

In some embodiments, one or more linkers utilized in an epigenetic editor provided herein is a non-peptide linker. For example, the linker may be a carbon bond, a disulfide bond, or carbon-heteroatom bond. In certain embodiments, the linker is a carbon-nitrogen bond of an amide linkage. In certain embodiments, the linker is a cyclic or acyclic, substituted or unsubstituted, or branched or unbranched aliphatic or heteroaliphatic linker.

In some embodiments, one or more linkers utilized in an epigenetic editor provided herein is polymeric (e.g., polyethylene, polyethylene glycol, polyamide, polyester, etc.). The linker may comprise, for example, a monomer, dimer, or polymer of aminoalkanoic acid; an aminoalkanoic acid (e.g., glycine, ethanoic acid, alanine, beta-alanine, 3-aminopropanoic acid, 4-aminobutanoic acid, 5-pentanoic acid, etc.); a monomer, dimer, or polymer of aminohexanoic acid (Ahx); or a polyethylene glycol moiety (PEG); or an aryl or heteroaryl moiety. In certain embodiments, the linker may be based on a carbocyclic moiety (e.g., cyclopentane or cyclohexane) or a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, alkyl halides, aryl halides, acyl halides, and isothiocyanates.

Various linker lengths and flexibilities can be employed between any two components of an epigenetic editor (e.g., between an effector domain (e.g., a repressor domain) and a DNA-binding domain (e.g., a Cas9 domain), between a first effector domain and a second effector domain, etc.). The linkers may range from very flexible linkers, such as glycine/serine-rich linkers, to more rigid linkers, in order to achieve the optimal length for effector domain activity for the specific application. In some embodiments, the more flexible linkers are glycine/serine-rich linkers (GS-rich linkers), where more than 45% (e.g., more than 48, 50, 55, 60, 70, 80, or 90%) of the residues are glycine or serine residues. Non-limiting examples of the GS-rich linkers are (GGGGS) n (SEQ ID NO: 664), (G) n, and W linker (SEQ ID NO: 637). In some embodiments, the more rigid linkers are in the form of the form (EAAAK) n (SEQ ID NO: 665), (SGGS) n (SEQ ID NO: 666), and (XP) n). In the aforementioned formulae of flexible and rigid linkers, n may be any integer between 1 and 30. In some embodiments, n is 1, 2, 3, 4, 5, 6, 7, 8, 9, 10, 11, 12, 13, 14, or 15. In some embodiments, the linker comprises a (GGS) n motif, wherein n is 1, 3, or 7. In some embodiments, the linker comprises a (GGGGS) n motif, wherein n is 4 (SEQ ID NO: 636).

In some embodiments, a linker in an epigenetic editor described herein comprises a nuclear localization signal, for example, with the amino acid sequence of any one of SEQ ID NOs: 644-649. In some embodiments, a linker in an epigenetic editor described herein comprises an expression tag, e.g., a detectable tag such as a green fluorescent protein.

### B. Nuclear Localization Signals

A fusion protein described herein may comprise one or more nuclear localization signals, and in certain embodiments, may comprise two or more nuclear localization signals. For example, the fusion protein may comprise 1, 2, 3, 4, 5, 6, 7, 8, 9, 10, or more nuclear localization signals. As used herein, a “nuclear localization signal” (NLS) is an amino acid sequence that directs proteins to the nucleus. In certain embodiments, the NLS may be an SV40 NLS (e.g., with the amino acid sequence of SEQ ID NO: 644). The fusion protein may comprise an NLS at its N-terminus, C-terminus, or both, and/or an NLS may be embedded in the middle of the fusion protein (e.g., at the N- or C-terminus of a DNA-binding domain or an effector domain).

In some embodiments, the fusion protein may comprise two NLSs. The fusion protein may comprise two NLSs at its N-terminus or C-terminus. The fusion protein may comprise one NLS located at its N-terminus and one NLS embedded in the middle of the fusion protein, or one NLS located at its C-terminus and one NLS embedded in the middle of the fusion protein. The fusion protein may comprise two NLSs embedded in the middle of the fusion protein.

In some embodiments, the fusion protein may comprise four NLSs. The fusion protein may comprise at least two (e.g., two, three, or four) NLSs at its N-terminus or C-terminus. The fusion protein may comprise at least one (e.g., one, two, three, or four) NLSs embedded in the middle of the fusion protein. In particular embodiments, the fusion protein may comprise two NLSs at its N-terminus and two NLSs at its C-terminus.

An NLS described herein may be an endogenous NLS sequence. In certain embodiments, an NLS described herein comprises the amino acid sequence of any one of SEQ ID NOs: 644-649, or a sequence at least 75%, 80%, 85%, 90%, 91%, 92%, 93%, 94%, 95%, 96%, 97%, 98%, or 99% identical to the selected sequence. In particular embodiments, the NLS comprises the amino acid sequence of SEQ ID NO: 644. Additional NLSs are known in the art.

In some embodiments, an epigenetic editor comprising a fusion protein that comprises at least one NLS at the N-terminus and at least one NLS at the C-terminus may increase the efficiency of the epigenetic editor by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor with a corresponding fusion protein that does not have at least one NLS at the N-terminus and at least one NLS at the C-terminus.

In some embodiments, an epigenetic editor comprising a fusion protein that comprises two NLSs at the N-terminus and two NLSs at the C-terminus may increase the efficiency of the epigenetic editor by at least 5%, at least 10%, at least 15%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 5,000%, at least 10,000%, at least 50,000%, at least 100,000%, or more as compared to an epigenetic editor with a corresponding fusion protein that does not have two NLSs at the N-terminus and two NLSs at the C-terminus.

### C. Tags

Epigenetic editors provided herein may comprise one or more additional sequences (“tags”) for tracking, detection, and localization of the editors. In some embodiments, the epigenetic editor comprises 1, 2, 3, 4, 5, 6, 7, 8, 9, 10 or more detectable tags. Each of the detectable tags may be the same or different.

For example, an epigenetic editor fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, poly-histidine tags (also referred to as histidine tags or His-tags), maltose binding protein (MBP)-tags, nus-tags, glutathione-S-transferase (GST)-tags, green fluorescent protein (GFP)-tags, thioredoxin-tags, S-tags, Softags (e.g., Softag 1 or Softag 3), strep-tags, biotin ligase tags, FlAsH tags, V5 tags, and SBP-tags. Additional suitable sequences will be apparent to those of skill in the art.

### D. Fusion Protein Configurations

A fusion protein of an epigenetic editor described herein may have its components structured in different configurations. For example, the DNA-binding domain may be at the C-terminus, the N-terminus, or in between two or more epigenetic effector domains or additional domains. In some embodiments, the DNA-binding domain is at the C-terminus of the epigenetic editor. In some embodiments, the DNA-binding domain is at the N-terminus of the epigenetic editor. In some embodiments, the DNA-binding domain is linked to one or more nuclear localization signals. In some embodiments, the DNA-binding domain is flanked by an epigenetic effector domain and/or an additional domain on both sides. In some embodiments, where “DBD” indicates DNA-binding domain and “ED” indicates effector domain, the epigenetic editor comprises the configuration of:

N′]-[ED1]-[DBD]-[ED2]-[C′
N′]-[ED1]-[DBD]-[ED2]-[ED3]-[C′
N′]-[ED1]-[ED2]-[DBD]-[ED3]-[C′
or
N′]-[ED1]-[ED2]-DBD]-[ED3]-[ED4]-[C′.

In some embodiments, an epigenetic editor comprises a DNA-binding domain (DBD), a DNA methyltransferase (DNMT) domain, and a transcriptional repressor (“repressor”) domain that represses or silences expression of a target gene. The DBD, DNMT, and transcriptional repressor domains may be any as described herein, in any combination. The DBD, DNMT domain, and repressor domain may be in any configuration, e.g., with any of said domains at the N-terminus, at the C-terminus, or in the middle of the fusion protein. In some embodiments, the epigenetic editor comprises a fusion protein with the configuration of:

N′]-[DNMT domain]-[DBD]-[repressor domain]-[C′
N′]-[repressor domain]-[DBD]-[DNMT domain]-[C′
N′]-[DNMT domain]-[repressor domain]- [DBD]-[C′
or
N′]-[repressor domain]-[DNMT domain]- [DBD]-[C′.

In some embodiments, a connecting structure “]-[” in any one of the epigenetic editor structures is a linker, e.g., a peptide linker; a detectable tag; a peptide bond; a nuclear localization signal; and/or a promoter or regulatory sequence. In an epigenetic editor structure, the multiple connecting structures “]-[” may be the same or may each be a different linker, tag, NLS, or peptide bond. In some embodiments, the DNMT domain may comprise any one of the domains in Table 7, or any combinations or homologs thereof. In particular embodiments, the DNMT domain comprises DNMT3A or a truncated version thereof, DNMT3L or a truncated version thereof, or both. In particular embodiments, the DBD is a catalytically inactive polynucleotide guided DNA-binding domain (e.g., a dCas9) or a ZFP domain. In certain embodiments, the repressor domain comprises any one of the domains shown in Table 5 or 6, or any combinations or homologs thereof. For example, the repressor domain may be a KRAB domain. In certain embodiments, the repressor domain is a ZFP28, ZN627, KAP1, MeCP2, HP1b, CBX8, CDYL2, TOX, Tox3, Tox4, EED, RBBP4, RCOR1, or SCML2 domain, or a fusion of two of said domains (e.g., a fusion of the N- and C-terminal regions of ZIM3 and KOX1 KRAB). In particular embodiments, the repressor domain is a KRAB domain from ZFP28, ZN627, ZIM3, or KOX1.

In some embodiments, the epigenetic editor comprises a configuration selected from

N′]-[DNMT3A-DNMT3L]-[DBD]-[repressor]-[C′
N′]-[repressor]-[DBD]-[DNMT3A-DNMT3L]-[C′
N′]-[repressor]-[DBD]-[DNMT3A]-[C′
N′]-[DNMT3A]-[DBD]-[repressor]-[C′
N′]-[repressor]-[DBD]-[DNMT3A]-[DNMT3L]-[C′
N′]-[DNMT3A]-[DNMT3L]-[DBD]-[repressor]-[C′
N′]-[DNMT3A]-[DBD]-[C′
N′]-[DBD]-[DNMT3A]-[C′
N′]-[DNMT3L]-[DBD]-[C′
N′]-[DBD]-[DNMT3L]-[C′

wherein [DNMT3A-DNMT3L] indicates that the DNMT3A and DNMT3L domains are directly fused via a peptide bond, and wherein the connecting structure]-[is any one of the linkers as described herein, a detectable tag, an affinity domain, a peptide bond, a nuclear localization signal, a promoter, and/or a regulatory sequence. The DBD, KRAB repressor, DNMT3A, and DNMT3L domains may be any as described herein, in any combination. For example, the DNMT3A and DNMT3L domains may be selected from those in Table 7. In particular embodiments, the DBD is a CRISPR-associated protein domain (e.g., dCas9) or a ZFP domain; the repressor domain is a KRAB domain derived from KOX1, ZIM3, ZFP28, or ZN627; the DNMT3A domain is a human DNMT3A domain; and the DNMT3L domain is a human or mouse DNMT3L domain; any combination of these components is also contemplated by the present disclosure.

In some embodiments, the epigenetic editor comprises a configuration selected from

N′]-[DNMT3A]-[DBD]-[SETDB1]-[C′
N′]-[DNMT3A]-[DNMT3L]-[DBD]-[SETDB1]-[C′
N′]-[DNMT3A-DNMT3L]-[DBD]-[SETDB1]-[C′
N′]-[SETDB1]-[DBD]-[DNMT3A]-[DNMT3L]-[C′
N′]-[SETDB1]-[DBD]-[DNMT3A]-[C′

wherein [DNMT3A-DNMT3L] indicates that the DNMT3A and DNMT3L domains are directly fused via a peptide bond, and wherein the connecting structure]-[is any one of the linkers as described herein, a detectable tag, an affinity domain, a peptide bond, a nuclear localization signal, a promoter, and/or a regulatory sequence. The DBD, SETDB1, DNMT3A, and DNMT3L domains may be any as described herein, in any combination. In particular embodiments, the DBD is a CRISPR-associated protein domain (e.g., dCas9) or a ZFP domain; the SETDB1 domain is derived from human SETDB1, ZIM3, ZFP28, or ZN627; the DNMT3A domain is a human DNMT3A domain; and the DNMT3L domain is a human or mouse DNMT3L domain; any combination of these components is also contemplated by the present disclosure.

Particular constructs contemplated herein include:

DNMT3A-DNMT3L-XTEN80-NLS-dCas9-NLS-XTEN16-KOX1 KRAB (Configuration 1),
DNMT3A-DNMT3L-XTEN80-NLS-ZFP domain-NLS-XTEN16-KOX1 KRAB
(Configuration 2),
NLS-DNMT3A-DNMT3L-XTEN80-dCas9-XTEN16-KOX1 KRAB-NLS (Configuration 3),
NLS-DNMT3A-DNMT3L-XTEN80-ZFP domain-XTEN16-KOX1 KRAB-NLS
(Configuration 4),
NLS-NLS-DNMT3A-DNMT3L-XTEN80-dCas9-XTEN16-KOX1 KRAB-NLS-NLS
(Configuration 5), and
NLS-NLS-DNMT3A-DNMT3L-XTEN80-ZFP domain-XTEN16-KOX1 KRAB-NLS-NLS
(Configuration 6).

The DNMT3L and DNMT3A may be derived from human parental proteins, mouse parental proteins, or any combination thereof. In certain embodiments, the DNMT3L and DNMT3A are derived from mouse and human parental proteins, respectively (mDNMT3L and hDNMT3A). In certain embodiments, the DNMT3L and DNMT3A are both derived from human parental proteins (hDNMT3L and hDNMT3A). In some embodiments, the dCas9 is dSpCas9. In some embodiments, the KOX1 is human KOX1. Also contemplated is any of Configurations 1-6 wherein the KOX1 KRAB domain is replaced by a ZFP28, ZN627, or ZIM3 KRAB domain. In some embodiments, the ZFP28, ZN627, and ZIM3 are human ZFP28, ZN627, and ZIM3, respectively. In particular embodiments, the fusion construct may have the configuration:

NLS-NLS-hDNMT3A-hDNMT3L-XTEN80-dCas9-XTEN16-KOX1 KRAB-NLS-NLS
(Configuration 7),
NLS-NLS-DNMT3A-DNMT3L-XTEN80-ZFP domain-XTEN16-KOX1 KRAB-NLS-NLS
(Configuration 8),
NLS-NLS-hDNMT3A-hDNMT3L-XTEN80-dCas9-XTEN16-ZFP28 KRAB-NLS-NLS
(Configuration 9),
NLS-NLS-DNMT3A-DNMT3L-XTEN80-ZFP domain-XTEN16-ZFP28 KRAB-NLS-NLS
(Configuration 10),
NLS-NLS-hDNMT3A-hDNMT3L-XTEN80-dCas9-XTEN16-ZN627 KRAB-NLS-NLS
(Configuration 11),
NLS-NLS-DNMT3A-DNMT3L-XTEN80-ZFP domain-XTEN16-ZN627 KRAB-NLS-NLS
(Configuration 12),
NLS-NLS-hDNMT3A-hDNMT3L-XTEN80-dCas9-XTEN16-ZIM3 KRAB-NLS-NLS
(Configuration 13), or
NLS-NLS-DNMT3A-DNMT3L-XTEN80-ZFP domain-XTEN16-ZIM3 KRAB-NLS-NLS
(Configuration 14).

In particular embodiments, a fusion construct described herein may have Configuration 1 and comprise SEQ ID NO: 658, or a sequence at least 75%, 80%, 85%, 90%, 91%, 92%, 93%, 94%, 95%, 96%, 97%, 98%, or 99% identical thereto. In SEQ ID NO: 658 below, the XTEN linkers are underlined, the W linker is bolded, underlined, and italicized, the NLS sequences are bolded, the DNMT3A sequence is italicized, the DNMT3L sequence is underlined and italicized, the dCas9 domain is bolded and italicized, and the KOX1 KRAB domain is underlined and bolded:

(SEQ ID NO: 658)
MNHDQEFDPPKVYPPVPAEKRKPIRVLSLFDGIATGLLVLKDLGIQVDRYIASEVCEDSITV
GMVRHQGKIMYVGDVRSVTQKHIQEWGPFDLVIGGSPCNDLSIVNPARKGLYEGTGRLFFEF
YRLLHDARPKEGDDRPFFWLFENVVAMGVSDKRDISRFLESNPVMIDAKEVSAAHRARYFWG
NLPGMNRPLASTVNDKLELQECLEHGRIAKFSKVRTITTRSNSIKQGKDQHFPVFMNEKEDI
LWCTEMERVFGFPVHYTDVSNMSRLARQRLLGRSWSVPVIRHLFAPLKEYFACVSSGNSNAN
SRGPSFSSGLVPLSLRGSHMGPMEIYKTVSAWKRQPVRVLSLFRNIDKVLKSLGFLESGSGS
GGGTLKYVEDVTNVVRRDVEKWGPFDLVYGSTQPLGSSCDRCPGWYMFQFHRILQYALPRQE
SQRPFFWIFMDNLLLTEDDQETTTRFLQTEAVTLQDVRGRDYQNAMRVWSNIPGLKSKHAPL
TPKEEEYLQAQVRSRSKLDAPKVDLLVKNCLLPLREYFKYFSQNSLPLGGPSSGAPPPSGGS
PAGSPTSTEEGTSESATPESGPGTSTEPSEGSAPGSPAGSPTSTEEGTSTEPSEGSAPGTST
EPSEPKKKRKVYMDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDELD
NEENEDILEDIVLTLTLFEDREMIEERLKTYAHLEDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDAIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDERKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSI
TGLYETRIDLSQLGGDPKKKRKVSGSETPGTSESATPESTGRTLVTFKDVFVDFTREEWKLL
DTAQQIVYRNVMLENYKNLVSLGYQLTKPDVILRLEKGEEP

In particular embodiments, a fusion construct described herein may have Configuration 2 and comprise SEQ ID NO: 659, or a sequence at least 75%, 80%, 85%, 90%, 91%, 92%, 93%, 94%, 95%, 96%, 97%, 98%, or 99% identical thereto. In SEQ ID NO: 659 below, the XTEN linkers are underlined, the W linker is bolded, underlined, and italicized, the NLS sequences are bolded and underlined, the DNMT3A sequence is italicized, the DNMT3L sequence is underlined and italicized, the ZFP domain is bolded, and the KOX1 KRAB domain is underlined and bolded. Variable amino acids represented by Xs are the amino acids of the DNA-recognition helix of the zinc finger and XX in italics may be either TR, LR or LK.

(SEQ ID NO: 659)
MNHDQEFDPPKVYPPVPAEKRKPIRVLSLFDGIATGLLVLKDLGIQVDRYIASEVCEDSITV
GMVRHQGKIMYVGDVRSVTQKHIQEWGPFDLVIGGSPCNDLSIVNPARKGLYEGTGRLFFEF
YRLLHDARPKEGDDRPFFWLFENVVAMGVSDKRDISRFLESNPVMIDAKEVSAAHRARYFWG
NLPGMNRPLASTVNDKLELQECLEHGRIAKFSKVRTITTRSNSIKQGKDQHFPVFMNEKEDI
LWCTEMERVFGFPVHYTDVSNMSRLARQRLLGRSWSVPVIRHLFAPLKEYFACVSSGNSNAN
SRGPSFSSGLVPLSLRGSHMGPMEIYKTVSAWKRQPVRVLSLFRNIDKVLKSLGFLESGSGS
GGGTLKYVEDVTNVVRRDVEKWGPFDLVYGSTQPLGSSCDRCPGWYMFQFHRILQYALPRQE
SQRPFFWIFMDNLLLTEDDQETTTRFLQTEAVTLQDVRGRDYQNAMRVWSNIPGLKSKHAPL
TPKEEEYLQAQVRSRSKLDAPKVDLLVKNCLLPLREYFKYFSQNSLPLGGPSSGAPPPSGGS
PAGSPTSTEEGTSESATPESGPGTSTEPSEGSAPGSPAGSPTSTEEGTSTEPSEGSAPGTST
EPSEPKKKRKVYSRPGERPFQCRICMRNFSXXXXXXXHXXTHTGEKPFQCRICMRNFSXXXX
XXXHXXTH[linker]PFQCRICMRNFSXXXXXXXHXXTHTGEKPFQCRICMRNFSXXXXXX
XHXXTH[linker]PFQCRICMRNFSXXXXXXXHXXTHTGEKPFQCRICMRNFSXXXXXXXH
XXTHLRGSPKKKRKVSGSETPGTSESATPESTGRTLVTFKDVFVDFTREEWKLLDTAQQIVY
RNVMLENYKNLVSLGYQLTKPDVILRLEKGEEP

In certain embodiments, the six “XXXXXXX” regions in SEQ ID NO: 659 comprise amino acid sequences that form a zinc finger. In the sequence above, [linker] represents a linker sequence. In some embodiments, one or both linker sequences may be TGSQKP (SEQ ID NO: 651). In some embodiments, one or both linker sequences may be TGGGGSQKP (SEQ ID NO: 652). In some embodiments, one linker sequence may have the amino acid sequence of SEQ ID NO: 651 and the other linker sequence may have the amino acid sequence of SEQ ID NO: 652. In some embodiments, the linker sequence is a minimum of 5 amino acids in length. In some embodiments, the linker sequence is a maximum of 250 amino acids in length

In particular embodiments, a fusion construct described herein may have Configuration 7 and comprise SEQ ID NO: 660, or a sequence at least 75%, 80%, 85%, 90%, 91%, 92%, 93%, 94%, 95%, 96%, 97%, 98%, or 99% identical thereto.

In particular embodiments, a fusion construct described herein may have Configuration 9 and comprise SEQ ID NO: 661, or a sequence at least 75%, 80%, 85%, 90%, 91%, 92%, 93%, 94%, 95%, 96%, 97%, 98%, or 99% identical thereto.

In particular embodiments, a fusion construct described herein may have Configuration 11 and comprise SEQ ID NO: 662, or a sequence at least 75%, 80%, 85%, 90%, 91%, 92%, 93%, 94%, 95%, 96%, 97%, 98%, or 99% identical thereto.

In particular embodiments, a fusion construct described herein may have Configuration 13 and comprise SEQ ID NO: 663, or a sequence at least 75%, 80%, 85%, 90%, 91%, 92%, 93%, 94%, 95%, 96%, 97%, 98%, or 99% identical thereto.

In some embodiments, a fusion construct described herein (e.g., the fusion construct of any one of Configurations 1-14) is within an expression construct that comprises a WPRE sequence, a polyadenylation site, or both. In certain embodiments, the WPRE sequence is in a 3′ noncoding region. In certain embodiments, the WPRE sequence is upstream from a poly-adenylation site. In particular embodiments, the expression construct comprises the fusion construct (e.g., of any one of Configurations 1-14) and a WPRE sequence in a 3′ noncoding region upstream from a polyadenylation site.

Multiple fusion proteins may be used to effect activation or repression of a target gene or multiple target genes. For example, an epigenetic editor fusion protein comprising a DNA-binding domain (e.g., a dCas9 domain) and an effector domain may be co-delivered with two or more guide polynucleotides (e.g., gRNAs), each targeting a different target DNA sequence. The target sites for two of the DNA-binding domains may be the same or in the vicinity of each other, or separated by, for example, about 100 base pairs, about 200 base pairs, about 300 base pairs, about 400 base pairs, about 500 base pairs, or about 600 or more base pairs. In addition, when targeting double-strand DNA, such as an endogenous gene locus, the guide polynucleotides may target the same or different strands (one or more to the positive strand and/or one or more to the negative strand).

In some embodiments, an epigenetic editor targeting CIITA is used in combination with epigenetic editor(s) targeting TRAC, TRBC, B2M, PDCD1, TIM-3, TIGIT, LAG3, CTLA4, AAVS1, CCR5, TET2, TGFBR2, A2AR, CISH, PTPN11, PTPN6, PTPA, PTPN2, JUNB, TOX, TOX2, NR4A1, NR4A2, NR4A3, MAP4K1, REL, IRF4, DGKA, PIK3CD, HLA-A, USP16, DCK, FAS, or any combination thereof.

### V. Target Sequences

An epigenetic editor herein may be directed to a target sequence in CIITA to effect epigenetic modification of the CIITA gene.

As used herein, a “target sequence,” a “target site,” or a “target region” is a nucleic acid sequence present in a gene of interest; in some instances, the target sequence may be outside but in the vicinity of the gene of interest wherein methylation or binding by a repressor of the target sequence represses expression of the gene. In some embodiments, the target sequence may be a hypomethylated or hypermethylated nucleic acid sequence.

The target sequence may be in any part of a target gene. In some embodiments, the target sequence is part of or near a noncoding sequence of the gene. In some embodiments, the target sequence is part of an exon of the gene. In some embodiments, the target sequence is part of or near a transcriptional regulatory sequence of the gene, such as a promoter or an enhancer. In some embodiments, the target sequence is adjacent to, overlaps with, or encompasses a CpG island. In certain embodiments, the target sequence is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs (bp) flanking a CIITA TSS. In certain embodiments, the target sequence is within 500 bp flanking the CIITA TSS. In certain embodiments, the target sequence is within 1000 bp flanking the CIITA TSS.

In some embodiments, the target sequence may hybridize to a guide polynucleotide sequence (e.g., gRNA) complexed with a fusion protein comprising a polynucleotide guided DNA-binding domain (e.g., a CRISPR protein such as dCas9) and effector domain(s). The guide polynucleotide sequence may be designed to have complementarity to the target sequence, or identity to the opposing strand of the target sequence. In some embodiments, the guide polynucleotide comprises a spacer sequence that is about 80%, 85%, 90%, 91%, 92%, 93%, 94%, 95%, 96%, 97%, 98% or 99% identical to a protospacer sequence in the target sequence. In particular embodiments, the guide polynucleotide comprises a spacer sequence that is 100% identical to a protospacer sequence in the target sequence.

In some embodiments, where the DNA-binding domain of an epigenetic editor described herein is a zinc finger array, the target sequence may be recognized by said zinc finger array.

In some embodiments, where the DNA-binding domain of an epigenetic editor described herein is a TALE, the target sequence may be recognized by said TALE.

A target sequence described herein may be specific to one copy of a target gene, or may be specific to one allele of a target gene. Accordingly, the epigenetic modification and modulation of expression thereof may be specific to one copy or one allele of the target gene. For example, an epigenetic editor may repress expression of a specific copy harboring a target sequence recognized by the DNA-binding domain (e.g., a copy associated with a disease or condition, or that harbors a mutation associated with a disease or condition).

In some embodiments, the target CIITA genomic region may fall within the sequence shown in SEQ ID NO: 1313 or 1314.

### VI. Epigenetic Modifications

An epigenetic editor described herein may perform sequence-specific epigenetic modification(s) (e.g., alteration of chemical modification(s)) of a target gene that harbors the target sequence. Such epigenetic modulation may be safer and more easily reversible than modulation due to gene editing, e.g., with generation of DNA double-strand breaks. In some embodiments, the epigenetic modulation may reduce or silence the target gene. In some embodiments, the modification is at a specific site of the target sequence. In some embodiments, the modification is at a specific allele of the target gene. Accordingly, the epigenetic modification may result in modulated (e.g., reduced) expression of one copy of a target gene harboring a specific allele, and not the other copy of the target gene. In some embodiments, the specific allele is associated with a disease, condition, or disorder.

In some embodiments, the epigenetic modification reduces or abolishes transcription of the target gene harboring the target sequence. In some embodiments, the epigenetic modification reduces or abolishes transcription of a copy of the target gene harboring a specific allele recognized by the epigenetic editor. In some embodiments, the epigenetic editor reduces the level of or eliminates expression of a protein encoded by the target gene. In some embodiments, the epigenetic editor reduces the level of or eliminates expression of a protein encoded by a copy of the target gene harboring a specific allele recognized by the epigenetic editor. The target CIITA gene may be epigenetically modified in vitro, ex vivo, or in vivo.

The effector domain of an epigenetic editor described herein may alter (e.g., deposit or remove) a chemical modification at a nucleotide of the target gene or at a histone associated with the target gene. The chemical modification may be altered at a single nucleotide or a single histone, or may be altered at 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides.

In some embodiments, an effector domain of an epigenetic editor described herein may alter a CpG dinucleotide within the target gene. In some embodiments, all CpG dinucleotides within 2000, 1500, 1000, 500, or 200 bps flanking a target sequence (e.g., in an alteration site as described herein) are altered according to a modification type described herein, as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more of the CpG dinucleotides are altered as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the CpG dinucleotides are altered as compared to the original state of the gene or the gene in a comparable cell not contacted with the epigenetic editor. In some embodiments, one single CpG dinucleotide is altered, as compared to the original state of the gene or the gene in a comparable cell not contacted with the epigenetic editor.

An effector domain of an epigenetic editor described herein may alter a histone modification state of a histone associated with or bound to the target gene. For example, an effector domain may deposit a modification on one or more lysine residues of histone tails of histones associated with the target gene. In some embodiments, the effector domain may result in deacetylation of one or more histone tails of histones associated with the target gene, thereby reducing or silencing expression of the target gene. In some embodiments, the histone modification state is a methylation state. For example, the effector domain may result in a H3K9, H3K27 or H4K20 methylation (e.g. one or more of a H3K9me2, H3K9me3, H3K27me2, H3K27me3, and H4K20me3 methylation) at one or more histone tails associated with the target gene, thereby reducing or silencing expression of the target gene.

In some embodiments, all histone tails of histones bound to DNA nucleotides within 2000, 1500, 1000, 500, or 200 bps flanking the target sequence are altered according to a modification type as described herein, as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of the bound histones are altered as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of the bound histones are altered as compared to the original state of the chromosome or the chromosome in a comparable cell not contacted with the epigenetic editor. For example, one single histone tail of the bound histones may be altered as compared to the original state of the chromosome or the chromosome in a comparable cell not contacted with the epigenetic editor. As another example, one single bound histone octamer may be altered as compared to the original state of the chromosome or the chromosome in a comparable cell not contacted with the epigenetic editor.

The chemical modification deposited at target gene DNA nucleotides or histone residues may be at or in close proximity to a target sequence in the target gene. In some embodiments, an effector domain of an epigenetic editor described herein alters a chemical modification state of a nucleotide or histone tail bound to a nucleotide 100-200, 200-300, 300-400, 400-55, 500-600, 600-700, or 700-800 nucleotides 5′ or 3′ to the target sequence in the target gene. In some embodiments, an effector domain alters a chemical modification state of a nucleotide or histone tail bound to a nucleotide within 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 nucleotides flanking the target sequence. As used herein, “flanking” refers to nucleotide positions 5′ to the 5′ end of and 3′ to the 3′ end of a particular sequence, e.g. a target sequence.

In some embodiments, an effector domain mediates or induces a chemical modification change of a nucleotide or a histone tail bound to a nucleotide distant from a target sequence. Such modification may be initiated near the target sequence, and may subsequently spread to one or more nucleotides in the target gene distant from the target sequence. For example, an effector domain may initiate alteration of a chemical modification state of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration may spread to one or more nucleotides at least 100, 200, 300, 400, 500, 600, 700, 800, 900, 1000, 1100, 1200, 1300, 1400, 1500, 1600, 1700, 1800, 1900, 2000, 2500, 3000, or more nucleotides from the target sequence in the target gene, either upstream or downstream of the target sequence. In certain embodiments, the chemical modification may be initiated at less than 2, 3, 5, 10, 20, 30, 40, 50, or 100 nucleotides in the target gene and spread to at least 100, 200, 300, 400, 500, 600, 700, 800, 900, 1000, 2000, or more nucleotides in the target gene. In some embodiments, the chemical modification spreads to nucleotides in the entire target gene. Additional proteins or transcription factors, for example, transcription repressors, methyltransferases, or transcription regulation scaffold proteins, may be involved in the spreading of the chemical modification. Alternatively, the epigenetic editor alone may be involved.

In some embodiments, an epigenetic editor described herein reduces expression of a target gene by at least about 20%, at least about 30%, at least about 40%, at least about 50%, at least about 60%, at least about 70%, at least about 80%, at least about 90%, at least about 95%, at least about 99%, or more, as measured by transcription of the target gene in a cell, a tissue, or a subject as compared to a control cell, control tissue, or a control subject (e.g., in the absence of the epigenetic editor). In some embodiments, the epigenetic editors described herein reduces expression of a copy of target gene by at least about 20%, at least about 30%, at least about 40%, at least about 50%, at least about 60%, at least about 70%, at least about 80%, at least about 90%, at least about 95%, at least about 99%, or more, as measured by transcription of the copy of the target gene in a cell, a tissue, or a subject as compared to a control cell, control tissue, or a control subject. In certain embodiments, the copy of the target gene harbors a specific sequence or allele recognized by the epigenetic editor. In particular embodiments, the epigenetically modified copy encodes a functional protein, and accordingly an epigenetic editor disclosed herein may reduce or abolish expression and/or function of the protein. For example, an epigenetic editor described herein may reduce expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90 -fold, or at least 100 fold in a cell, a tissue, or a subject as compared to a control cell, control tissue, or a control subject.

Modulation of target gene expression can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP; changes in signal transduction; changes in phosphorylation and dephosphorylation; changes in receptor-ligand interactions; changes in concentrations of second messengers such as, for example, cGMP, CAMP, IP3, and Ca2+; changes in cell growth; changes in neovascularization; and/or changes in any functional effect of gene expression. Measurements can be made in vitro, in vivo, and/or ex vivo, and can be made by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays, changes in intracellular second messengers such as cGMP and inositol triphosphate (IP3), changes in intracellular calcium levels; cytokine release, and the like.

Methods for determining the expression level of a gene, for example the target of an epigenetic editor, may include, e.g., determining the transcript level of a gene by reverse transcription PCR, quantitative RT-PCR, droplet digital PCR (ddPCR), Northern blot, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Levels of protein expressed from a gene may be determined, e.g., by Western blotting, enzyme linked immuno-absorbance assays, mass-spectrometry, immunohistochemistry, or flow cytometry analysis. Gene expression product levels may be normalized to an internal standard such as total messenger ribonucleic acid (mRNA) or the expression level of a particular gene, e.g., a housekeeping gene.

In some embodiments, the effect of an epigenetic editor in modulating target gene expression may be examined using a reporter system. For example, an epigenetic editor may be designed to target a reporter gene encoding a reporter protein, such as a fluorescent protein. Expression of the reporter gene in such a model system may be monitored by, e.g., flow cytometry, fluorescence-activated cell sorting (FACS), or fluorescence microscopy. In some embodiments, a population of cells may be transfected with a vector that harbors a reporter gene. The vector may be constructed such that the reporter gene is expressed when the vector transfects a cell. Suitable reporter genes include genes encoding fluorescent proteins, for example green, yellow, cherry, cyan or orange fluorescent proteins. The population of cells carrying the reporter system may be transfected with DNA, mRNA, or vectors encoding the epigenetic editor targeting the reporter gene.

### VII. Epigenetically Modified Cells

In one aspect, the present disclosure provides cells that have been modified using one or more epigenetic editor(s) described herein. In some embodiments, nucleic acid molecule(s) encoding said epigenetic editor(s) or component(s) thereof are administered to the cells. Any type of cell may be modified as described herein. The cells may be modified in vitro, in vivo, or ex vivo. Cells suitable for modification may be procured from a patient or a healthy donor.

In some embodiments, the cell is an immune cell. Immune cells may include T cells, B cells, natural killer (NK) cells, dendritic cells, and monocytes/macrophages. In some embodiments, the cell is an alpha/beta T cell. In some embodiments, the cell is a gamma/delta T cell. In some embodiments, the cell is a cytotoxic T cell, e.g., a CD8⁺ cytotoxic T cell. In some embodiments, the cell is a T helper cell, e.g., a CD4⁺ T helper cell. In some embodiments, the cell is a regulatory T cell. In some embodiments, the cell is an NK cell. In some embodiments, the cell is a dendritic cell. In some embodiments, the cell is a macrophage.

In some embodiments, the cell is a stem cell. A “stem cell” refers to an undifferentiated cell which is capable of indefinitely giving rise to more stem cells of the same type, and from which other specialized cells may arise by differentiation. Adult stem cells are usually multipotent, while induced or embryonic-derived stem cells are pluripotent.

In some embodiments, the cell is a progenitor cell. A “progenitor cell” refers to a cell which is able to differentiate to form one or more types of cells, but has limited self-renewal in vitro and in vivo.

In some embodiments, the cell is capable of differentiating into an immune cell described above. The cell may be, for example, an embryonic stem cell (ESC), a hematopoietic stem cell (HSC), a hematopoietic progenitor cell (HPC), or a hematopoietic stem and progenitor cell (HSPC). A “hematopoietic stem and progenitor cell” or “HSPC” refers to a cell which expresses the antigenic marker CD34 (CD34+). In particular embodiments, the term “HSPC” refers to a cell identified by the presence of the antigenic marker CD34 (CD34+) and the absence of lineage (lin) markers. The population of cells that are CD34+ and/or Lin includes hematopoietic stem cells and hematopoietic progenitor cells.

In some embodiments, the cell is an induced pluripotent stem cell (iPSC) reprogrammed from a somatic cell such as a T cell.

In some embodiments, the cell is obtained from umbilical cord blood of a healthy donor. In some embodiments, the cell is obtained from adult peripheral blood or mobilized from the bone marrow of a healthy donor.

In some embodiments, a cell as described above is modified by a method comprising transfecting the cell with a system comprising (a) one or more epigenetic editor(s) described herein, or (b) nucleic acid molecule(s) encoding said epigenetic editor(s). In certain embodiments, the modified cell is a T cell. In some embodiments, the modified T cell expresses one or more epigenetic editor(s) that are able to selectively reduce or silence the expression of one or more target gene(s) in the cell. In particular embodiments, the target gene is CIITA. In some embodiments, the T cells are modified ex vivo. The modified T cell may, in some embodiments, further express an engineered TCR or CAR directed against at least one antigen expressed at the surface of a target cell (e.g., a malignant or infected cell). In some embodiments, the modified T cell does not express at least one gene encoding an endogenous TCR component. In particular embodiments, the modified T cells are non-alloreactive. In particular embodiments, the modified T cells are particularly suitable for allogeneic transplantation.

### VII. Pharmaceutical Compositions

In one aspect, the present disclosure provides a pharmaceutical composition comprising as an active ingredient (or as the sole active ingredient) one or more epigenetic editors described herein or component(s) (e.g., fusion proteins and/or guide polynucleotides) thereof, or nucleic acid molecule(s) encoding said epigenetic editors or component(s) thereof. For example, a pharmaceutical composition may comprise nucleic acid molecule(s) encoding the fusion protein(s) (and guide polynucleotides, where applicable) of an epigenetic editor described herein. In some embodiments, separate pharmaceutical compositions comprise the fusion protein(s) and the guide polynucleotide(s).

In one aspect, the present disclosure provides a pharmaceutical composition comprising as an active ingredient (or as the sole active ingredient) cells that have undergone epigenetic modification(s) mediated or induced by (a) one or more epigenetic editor(s) provided herein, e.g., wherein nucleic acid molecule(s) encoding said epigenetic editor(s) were administered to said cells ex vivo.

Generally, the epigenetic editors described herein or component(s) thereof, or nucleic acid molecule(s) encoding said epigenetic editors or component(s) thereof, or cells modified by the epigenetic editors of the present disclosure, are suitable to be administered as a formulation in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives, or buffers, which enhance the shelf life or effectiveness of the antibody.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. The pharmaceutical compositions described herein may be administered to a subject, e.g., subcutaneously, intradermally, intratumorally, intranodally, intramuscularly, intravenously, intralymphatically, or intraperitoneally. In particular embodiments, a pharmaceutical composition of the present disclosure is administered intravenously to the subject.

### IX. Delivery Methods

In some embodiments, the epigenetic editor or its component(s) are introduced to target cells in the form of nucleic acid molecule(s) encoding the epigenetic editor or its component(s); accordingly, the pharmaceutical compositions herein comprise the nucleic acid molecule(s). Such nucleic acid molecule(s) may be, for example, DNA, RNA, or mRNA, and/or modified nucleic acid sequence(s) (e.g., with chemical modifications, a 5′ cap, or one or more 3′ modifications). In some embodiments, the nucleic acid molecule(s) may be delivered as naked DNA or RNA, for instance by means of transfection or electroporation, or can be conjugated to molecules (e.g., N-acetylgalactosamine) promoting uptake by target cells. In some embodiments, the nucleic acid molecule(s) may be in nucleic acid expression vector(s), which may include expression control sequences such as promoters, enhancers, transcription signal sequences, transcription termination sequences, introns, polyadenylation signals, Kozak consensus sequences, internal ribosome entry sites (IRES), etc. Such expression control sequences are well known in the art. A vector may also comprise a sequence encoding a signal peptide (e.g., for nuclear localization, nucleolar localization, or mitochondrial localization), associated with (e.g., inserted into or fused to) a sequence coding for a protein.

Examples of vectors include, but are not limited to, plasmid vectors; viral vectors based on vaccinia virus, poliovirus, adenovirus, adeno-associated virus, SV40, herpes simplex virus, human immunodeficiency virus, retrovirus (e.g., Murine Leukemia Virus, or spleen necrosis virus, vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and other recombinant vectors. In certain embodiments, the vector is a plasmid or a viral vector. Viral particles or virus-like particles (VLPs) may also be used to deliver nucleic acid molecule(s) encoding epigenetic editors or component(s) thereof as described herein. For example, “empty” viral particles can be assembled to contain any suitable cargo. Viral vectors and viral particles may also be engineered to incorporate targeting ligands to alter target tissue specificity.

In certain embodiments, an epigenetic editor as described herein or component(s) thereof are encoded by nucleic acid sequence(s) present in one or more viral vectors, or a suitable capsid protein of any viral vector. Examples of viral vectors include adeno-associated viral vectors (e.g., derived from AAV3, AAV3b, AAV4, AAV5, AAV6, AAV7, AAV8, AAV9, AAVrh8, AAV10, and/or variants thereof); retroviral vectors (e.g., Maloney murine leukemia virus, MML-V), adenoviral vectors (e.g., AD100), lentiviral vectors (e.g., HIV and FIV-based vectors), and herpesvirus vectors (e.g., HSV-2).

In some embodiments, delivery involves an adeno-associated virus (AAV) vector. AAV vector delivery may be particularly useful where the DNA-binding domain of an epigenetic editor fusion protein is a zinc finger array. Without wishing to be bound by any theory, the smaller size of zinc finger arrays compared to larger DNA-binding domains such as Cas protein domains may allow such a fusion protein to be conveniently packed in viral vectors such as an AAV vector.

Any AAV serotype, e.g., human AAV serotype, can be used for an AAV vector as described herein, including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), and AAV serotype 11 (AAV11), as well as variants thereof. In some embodiments, an AAV variant has at least 90%, 91%, 92%, 93%, 94%, 95%, 96%, 97%, 98%, or 99% amino acid sequence identity to a wildtype AAV. In certain embodiments, the AAV variant may be engineered such that its capsid proteins have reduced immunogenicity or enhanced transduction ability in humans. In some instances, one or more regions of at least two different AAV serotype viruses are shuffled and reassembled to generate a chimeric variant. For example, a chimeric AAV may comprise inverted terminal repeats (ITRs) that are of a heterologous serotype compared to the serotype of the capsid. The resulting chimeric AAV can have a different antigenic reactivity or recognition compared to its parental serotypes. In some embodiments, a chimeric variant of an AAV includes amino acid sequences from 2, 3, 4, 5, or more different AAV serotypes.

Non-viral systems are also contemplated for delivery as described herein. Non-viral systems include, but are not limited to, nucleic acid transfection methods including electroporation, sonoporation, calcium phosphate transfection, microinjection, DNA biolistics, lipid-mediated transfection, transfection through heat shock, compacted DNA-mediated transfection, lipofection, cationic agent-mediated transfection, and transfection with liposomes, immunoliposomes, exosomes, or cationic facial amphiphiles (CFAs). In certain embodiments, one or more mRNAs encoding epigenetic editor fusion proteins as described herein may be co-electroporated with one or more guide polynucleotides (e.g., gRNAs) as described herein. One important category of non-viral nucleic acid vectors is nanoparticles, which can be organic (e.g., lipid) or inorganic (e.g., gold). For instance, organic (e.g. lipid and/or polymer) nanoparticles can be suitable for use as delivery vehicles in certain embodiments of this disclosure.

In some embodiments, delivery is accomplished using a lipid nanoparticle (LNP). LNP compositions are typically sized on the order of micrometers or smaller and may include a lipid bilayer. In some embodiments, an LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm. Nanoparticle compositions encompass lipid nanoparticles (LNPs), liposomes (e.g., lipid vesicles), and lipoplexes.

An LNP as described herein may be made from cationic, anionic, or neutral lipids. In some embodiments, an LNP may comprise neutral lipids, such as the fusogenic phospholipid 1,2-Dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. The lipids may be combined in any molar ratios to produce the LNP. In some embodiments, the LNP is a T cell-targeting (e.g., preferentially or specifically targeting the T cell) LNP.

### X. Therapeutic Uses of Epigenetic Editors and Modified Cells

The present disclosure also provides methods for treating or preventing a condition in a subject, comprising administering to the subject a) one or more epigenetic editor(s) as described herein, b) nucleic acid molecule(s) encoding the epigenetic editor(s), c) cells modified by the epigenetic editor(s), or d) pharmaceutical compositions comprising any of a)-c).

In one aspect, the epigenetic editor may effect an epigenetic modification of a target polynucleotide sequence in a target gene associated with a disease, condition, or disorder in the subject, thereby modulating expression of the target gene to treat or prevent the disease, condition, or disorder. In some embodiments, the epigenetic editor reduces the expression of the target gene to an extent sufficient to achieve a desired effect, e.g., a therapeutically relevant effect such as the prevention or treatment of the disease, condition, or disorder.

In one aspect, a cell (e.g., an allogeneic cell) modified by one or more epigenetic editor(s) of the present disclosure may be administered as a medicament to a subject with a disease, condition, or disorder, thereby treating the disease, condition, or disorder. In some embodiments, the subject is administered allogeneic T cells which have been epigenetically modified as described herein, e.g., to have reduced or silenced CIITA expression. In some embodiments, the modified T cells further express an engineered TCR or CAR directed against at least one antigen expressed at the surface of a target cell (e.g., a malignant or infected cell). In some embodiments, the modified T cells do not express at least one gene encoding an endogenous TCR component.

In some embodiments, the subject may be a mammal, e.g., a human. In some embodiments, the subject is selected from a non-human primate such as chimpanzee, cynomolgus monkey, or macaque, and other ape and monkey species.

### XII. Definitions

The term “nucleic acid” as used herein refers to any oligonucleotide or polynucleotide containing nucleotides (e.g., deoxyribonucleotides or ribonucleotides) in either single- or double-strand form, and includes DNA and RNA. “Nucleotides” contain a sugar deoxyribose (DNA) or ribose (RNA), a base, and a phosphate group, and are linked together through the phosphate groups. “Bases” include purines and pyrimidines, which include natural compounds such as adenine, thymine, guanine, cytosine, uracil, inosine, and natural analogs; as well as synthetic derivatives of purines and pyrimidines, which include, but are not limited to, modified versions which place new reactive groups such as amines, alcohols, thiols, carboxylates, alkylhalides, etc. Nucleic acids may contain known nucleotide analogs and/or modified backbone residues or linkages, which may be synthetic, naturally occurring, and non-naturally occurring. Such nucleotide analogs, modified residues, and modified linkages are well known in the art, and may provide a nucleic acid molecule with enhanced cellular uptake, reduced immunogenicity, and/or increased stability in the presence of nucleases.

As used herein, an “isolated” or “purified” nucleic acid molecule is a nucleic acid molecule that exists apart from its native environment. For example, an “isolated” or “purified” nucleic acid molecule (1) has been separated away from the nucleic acids of the genomic DNA or cellular RNA of its source of origin; and/or (2) does not occur in nature. In some embodiments, an “isolated” or “purified” nucleic acid molecule is a recombinant nucleic acid molecule.

It will be understood that in addition to the specific proteins and nucleic acid molecules mentioned herein, the present disclosure also contemplates the use of variants, derivatives, homologs, and fragments thereof. A variant of any given sequence may have the specific sequence of residues (whether amino acid or nucleic acid residues) modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring sequence (in some embodiments, no more than 1, 2, 3, 4, 5, 6, 7, 8, 9, 10, 15, or 20 residues). For specific proteins described herein (e.g., KRAB, dCas9, DNMT3A, and DNMT3L proteins described herein), the present disclosure also contemplates any of the protein's naturally occurring forms, or variants or homologs that retain at least one of its endogenous functions (e.g., at least 50%, 60%, 70%, 80%, 90%, 85%, 96%, 97%, 98%, or 99% of its function as compared to the specific protein described).

As used herein, a homologue of any polypeptide or nucleic acid sequence contemplated herein includes sequences having a certain homology with the wildtype amino acid and nucleic sequence. A homologous sequence may include a sequence, e.g. an amino acid sequence which may be at least 50%, 55%, 65%, 75%, 85%, 90%, 91%, 92%<93%, 94%, 95%, 96%, 97%, 98%, or 99% identical to the subject sequence. The term “percent identical” in the context of amino acid or nucleotide sequences refers to the percent of residues in two sequences that are the same when aligned for maximum correspondence. In some embodiments, the length of a reference sequence aligned for comparison purposes is at least 30%, (e.g., at least 40, 50, 60, 70, 80, or 90%, or 100%) of the reference sequence. Sequence identity may be measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

The percent identity of two nucleotide or polypeptide sequences is determined by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90%) of the reference sequence.

It will be understood that the numbering of the specific positions or residues in polypeptide sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

The term “modulate” or “alter” refers to a change in the quantity, degree, or extent of a function. For example, an epigenetic editor as described herein may modulate the activity of a promoter sequence by binding to a motif within the promoter, thereby inducing, enhancing, or suppressing transcription of a gene operatively linked to the promoter sequence. As other examples, an epigenetic editor as described herein may block RNA polymerase from transcribing a gene, or may inhibit translation of an mRNA transcript. The terms “inhibit,” “repress,” “suppress,” “silence” and the like, when used in reference to an epigenetic editor or a component thereof as described herein, refers to decreasing or preventing the activity (e.g., transcription) of a nucleic acid sequence (e.g., a target gene) or protein relative to the activity of the nucleic acid sequence or protein in the absence of the epigenetic editor or component thereof. The term may include partially or totally blocking activity, or preventing or delaying activity. The inhibited activity may be, e.g., 10%, 20%, 30%, 40%, 50%, 60%, 70%, 75%, 80%, 85%, 90%, 91%, 92%, 93%, 94%, 95%, 96%, 97%, 98%, or 99% less than that of a control, or may be, e.g., at least 1.5-fold, 2-fold, 3-fold, 4-fold, 5-fold, or 10-fold less than that of a control.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the given value. Where particular values are described in the application and claims, unless otherwise stated, the term “about” should be assumed to mean an acceptable error range for the particular value.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. Unless otherwise indicated, the recitation of a listing of elements herein includes any of the elements singly or in any combination. The recitation of an embodiment herein includes that embodiment as a single embodiment, or in combination with any other embodiment(s) herein. All publications, patents, patent applications, and other references mentioned herein are incorporated by reference in their entirety. To the extent that references incorporated by reference contradict the disclosure contained in the specification, the specification is intended to supersede and/or take precedence over any such contradictory material. Although a number of documents are cited herein, this citation does not constitute an admission that any of these documents forms part of the common general knowledge in the art.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that the present disclosure may be better understood, the following examples are set forth. These examples are for purposes of illustration only and are not to be construed as limiting the scope of the present disclosure in any manner.

### EXAMPLES

### Example 1: Fusion Protein Design and Synthesis

A fusion protein comprising dCas9, DNMT3A, DNMT3L, and KOX1 KRAB (“CRISPR-off”) was produced. From N terminus to C terminus, the protein had the following functional domains and linkers: huDNMT3A-linker-huDNMT3L-XTEN80-NLS-dSpCas9-NLS-XTEN16-huKOX1 KRAB (SEQ ID NO: 658). The CRISPR-off plasmid construct is described in Nuñez et al., Cell (2021) 184 (9): 2503-19. ZF fusion proteins (“ZF-off”) comprising DNMT3A, 3L, and KOX1 KRAB were also produced. These fusion proteins had the following general structure: huDNMT3A-linker-huDNMT3L-XTEN80-NLS-ZFP domain-NLS-XTEN16-huKOX1 Krab (SEQ ID NO: 659).

### Example 2: Selection of CIITA Regions for gRNA Targeting

gRNAs targeting genomic regions within +/−750 bp of the primary canonical CIITA transcriptional start site (TSS1) (chr16: 10877202) and within-50/+280 bp of an alternative, downstream transcriptional start site (TSS2) (chr16: 10878968) were computationally designed using the Benchling gRNA platform for human (GRCh38). gRNAs containing poly-TTTT sequences were first discarded. gRNA off-target analysis using CasOFFinder (Bae et al., Bioinformatics (2014) 30 (10): 1473-5) was performed. gRNAs were discarded if they matched to multiple locations across the target genome.

A final set of 239 gRNA sequences targeting TSS1 and 40 gRNA sequences targeting TSS2 was selected for the primary screen in primary human T cells (Table 8; see Table 2 and Table 3 for gRNA target sequences and targeting domain sequences, respectively). DNA plasmids containing coding sequences for the gRNAs under the control of a U6 promoter were ordered from a vendor.

TABLE 8
Selected CIITA gRNAs and Target Sequences

| | Target | TSS1 | | Chr. 16 |
|---|---|---|---|---|
| gRNA No. | Sequence No. | Distance | START | Strand |
| gRNA001 | TAR908 | −724 | 10876478 | + |
| gRNA002 | TAR916 | −657 | 10876545 | − |
| gRNA003 | TAR924 | −597 | 10876605 | + |
| gRNA004 | TAR932 | −557 | 10876645 | + |
| gRNA005 | TAR940 | −464 | 10876738 | + |
| gRNA006 | TAR948 | −432 | 10876770 | + |
| gRNA007 | TAR956 | −371 | 10876831 | + |
| gRNA008 | TAR964 | −330 | 10876872 | − |
| gRNA009 | TAR972 | −240 | 10876962 | − |
| gRNA010 | TAR979 | −179 | 10877023 | + |
| gRNA011 | TAR987 | −146 | 10877056 | + |
| gRNA012 | TAR909 | −682 | 10876520 | − |
| gRNA013 | TAR917 | −654 | 10876548 | + |
| gRNA014 | TAR925 | −596 | 10876606 | + |
| gRNA015 | TAR933 | −556 | 10876646 | + |
| gRNA016 | TAR941 | −463 | 10876739 | + |
| gRNA017 | TAR949 | −401 | 10876801 | + |
| gRNA018 | TAR957 | −368 | 10876834 | + |
| gRNA019 | TAR965 | −326 | 10876876 | − |
| gRNA020 | TAR973 | −239 | 10876963 | − |
| gRNA021 | TAR980 | −178 | 10877024 | + |
| gRNA022 | TAR183 | −144 | 10877058 | + |
| gRNA023 | TAR910 | −672 | 10876530 | + |
| gRNA024 | TAR918 | −654 | 10876548 | − |
| gRNA025 | TAR926 | −570 | 10876632 | + |
| gRNA026 | TAR934 | −544 | 10876658 | − |
| gRNA027 | TAR942 | −456 | 10876746 | − |
| gRNA028 | TAR950 | −390 | 10876812 | − |
| gRNA029 | TAR958 | −367 | 10876835 | + |
| gRNA030 | TAR966 | −325 | 10876877 | + |
| gRNA031 | TAR974 | −238 | 10876964 | − |
| gRNA032 | TAR981 | −177 | 10877025 | + |
| gRNA033 | TAR988 | −116 | 10877086 | + |
| gRNA034 | TAR911 | −669 | 10876533 | + |
| gRNA035 | TAR919 | −628 | 10876574 | + |
| gRNA036 | TAR927 | −570 | 10876632 | − |
| gRNA037 | TAR935 | −541 | 10876661 | + |
| gRNA038 | TAR943 | −455 | 10876747 | + |
| gRNA039 | TAR951 | −389 | 10876813 | − |
| gRNA040 | TAR959 | −355 | 10876847 | + |
| gRNA041 | TAR967 | −318 | 10876884 | + |
| gRNA042 | TAR975 | −237 | 10876965 | − |
| gRNA043 | TAR982 | −174 | 10877028 | + |
| gRNA044 | TAR989 | −115 | 10877087 | + |
| gRNA045 | TAR912 | −668 | 10876534 | − |
| gRNA046 | TAR920 | −627 | 10876575 | + |
| gRNA047 | TAR928 | −569 | 10876633 | + |
| gRNA048 | TAR936 | −496 | 10876706 | + |
| gRNA049 | TAR944 | −450 | 10876752 | + |
| gRNA050 | TAR952 | −388 | 10876814 | − |
| gRNA051 | TAR960 | −338 | 10876864 | + |
| gRNA052 | TAR968 | −307 | 10876895 | + |
| gRNA053 | TAR976 | −205 | 10876997 | + |
| gRNA054 | TAR983 | −173 | 10877029 | + |
| gRNA055 | TAR990 | −114 | 10877088 | + |
| gRNA056 | TAR913 | −665 | 10876537 | − |
| gRNA057 | TAR921 | −613 | 10876589 | − |
| gRNA058 | TAR929 | −569 | 10876633 | − |
| gRNA059 | TAR937 | −493 | 10876709 | + |
| gRNA060 | TAR945 | −449 | 10876753 | + |
| gRNA061 | TAR953 | −382 | 10876820 | + |
| gRNA062 | TAR961 | −335 | 10876867 | + |
| gRNA063 | TAR969 | −293 | 10876909 | − |
| gRNA064 | TAR182 | −200 | 10877002 | − |
| gRNA065 | TAR984 | −166 | 10877036 | + |
| gRNA066 | TAR991 | −102 | 10877100 | + |
| gRNA067 | TAR914 | −665 | 10876537 | + |
| gRNA068 | TAR922 | −612 | 10876590 | − |
| gRNA069 | TAR930 | −568 | 10876634 | − |
| gRNA070 | TAR938 | −492 | 10876710 | + |
| gRNA071 | TAR946 | −443 | 10876759 | + |
| gRNA072 | TAR954 | −375 | 10876827 | + |
| gRNA073 | TAR962 | −332 | 10876870 | − |
| gRNA074 | TAR970 | −282 | 10876920 | − |
| gRNA075 | TAR977 | −193 | 10877009 | − |
| gRNA076 | TAR985 | −165 | 10877037 | + |
| gRNA077 | TAR915 | −664 | 10876538 | + |
| gRNA078 | TAR923 | −600 | 10876602 | + |
| gRNA079 | TAR931 | −563 | 10876639 | − |
| gRNA080 | TAR939 | −469 | 10876733 | − |
| gRNA081 | TAR947 | −438 | 10876764 | + |
| gRNA082 | TAR955 | −374 | 10876828 | + |
| gRNA083 | TAR963 | −331 | 10876871 | − |
| gRNA084 | TAR971 | −265 | 10876937 | + |
| gRNA085 | TAR978 | −184 | 10877018 | + |
| gRNA086 | TAR986 | −158 | 10877044 | + |
| gRNA087 | TAR184 | −97 | 10877105 | − |
| gRNA088 | TAR270 | −45 | 10877157 | + |
| gRNA089 | TAR186 | 18 | 10877220 | + |
| gRNA090 | TAR273 | 49 | 10877251 | + |
| gRNA091 | TAR277 | 87 | 10877289 | − |
| gRNA092 | TAR1018 | 134 | 10877336 | + |
| gRNA093 | TAR1025 | 166 | 10877368 | + |
| gRNA094 | TAR1032 | 246 | 10877448 | + |
| gRNA095 | TAR1040 | 260 | 10877462 | + |
| gRNA096 | TAR1048 | 301 | 10877503 | + |
| gRNA097 | TAR1056 | 352 | 10877554 | − |
| gRNA098 | TAR992 | −101 | 10877101 | + |
| gRNA099 | TAR999 | −44 | 10877158 | + |
| gRNA100 | TAR1004 | 18 | 10877220 | + |
| gRNA101 | TAR274 | 54 | 10877256 | − |
| gRNA102 | TAR1013 | 89 | 10877291 | + |
| gRNA103 | TAR188 | 136 | 10877338 | + |
| gRNA104 | TAR1026 | 170 | 10877372 | + |
| gRNA105 | TAR1033 | 247 | 10877449 | + |
| gRNA106 | TAR1041 | 264 | 10877466 | + |
| gRNA107 | TAR1049 | 303 | 10877505 | − |
| gRNA108 | TAR1057 | 355 | 10877557 | − |
| gRNA109 | TAR993 | −91 | 10877111 | − |
| gRNA110 | TAR1000 | −33 | 10877169 | + |
| gRNA111 | TAR1005 | 28 | 10877230 | + |
| gRNA112 | TAR275 | 56 | 10877258 | + |
| gRNA113 | TAR1014 | 93 | 10877295 | + |
| gRNA114 | TAR1019 | 136 | 10877338 | − |
| gRNA115 | TAR1027 | 171 | 10877373 | + |
| gRNA116 | TAR1034 | 248 | 10877450 | + |
| gRNA117 | TAR1042 | 265 | 10877467 | + |
| gRNA118 | TAR1050 | 317 | 10877519 | − |
| gRNA119 | TAR1058 | 359 | 10877561 | + |
| gRNA120 | TAR994 | −88 | 10877114 | − |
| gRNA121 | TAR185 | −25 | 10877177 | + |
| gRNA122 | TAR1006 | 37 | 10877239 | + |
| gRNA123 | TAR1010 | 59 | 10877261 | + |
| gRNA124 | TAR278 | 94 | 10877296 | − |
| gRNA125 | TAR1020 | 143 | 10877345 | − |
| gRNA126 | TAR1028 | 189 | 10877391 | − |
| gRNA127 | TAR1035 | 252 | 10877454 | + |
| gRNA128 | TAR1043 | 271 | 10877473 | + |
| gRNA129 | TAR1051 | 336 | 10877538 | + |
| gRNA130 | TAR1059 | 366 | 10877568 | + |
| gRNA131 | TAR995 | −61 | 10877141 | − |
| gRNA132 | TAR1001 | −19 | 10877183 | − |
| gRNA133 | TAR1007 | 38 | 10877240 | + |
| gRNA134 | TAR276 | 60 | 10877262 | + |
| gRNA135 | TAR1015 | 94 | 10877296 | + |
| gRNA136 | TAR1021 | 148 | 10877350 | − |
| gRNA137 | TAR1029 | 205 | 10877407 | + |
| gRNA138 | TAR1036 | 253 | 10877455 | + |
| gRNA139 | TAR1044 | 288 | 10877490 | + |
| gRNA140 | TAR1052 | 339 | 10877541 | + |
| gRNA141 | TAR1060 | 377 | 10877579 | − |
| gRNA142 | TAR996 | −60 | 10877142 | − |
| gRNA143 | TAR1002 | −18 | 10877184 | − |
| gRNA144 | TAR1008 | 39 | 10877241 | + |
| gRNA145 | TAR1011 | 61 | 10877263 | + |
| gRNA146 | TAR279 | 107 | 10877309 | − |
| gRNA147 | TAR1022 | 149 | 10877351 | − |
| gRNA148 | TAR1030 | 229 | 10877431 | − |
| gRNA149 | TAR1037 | 254 | 10877456 | + |
| gRNA150 | TAR1045 | 289 | 10877491 | + |
| gRNA151 | TAR1053 | 340 | 10877542 | + |
| gRNA152 | TAR1061 | 395 | 10877597 | + |
| gRNA153 | TAR997 | −58 | 10877144 | + |
| gRNA154 | TAR1003 | −14 | 10877188 | + |
| gRNA155 | TAR1009 | 44 | 10877246 | − |
| gRNA156 | TAR187 | 76 | 10877278 | + |
| gRNA157 | TAR1016 | 118 | 10877320 | + |
| gRNA158 | TAR1023 | 158 | 10877360 | + |
| gRNA159 | TAR189 | 237 | 10877439 | − |
| gRNA160 | TAR1038 | 255 | 10877457 | + |
| gRNA161 | TAR1046 | 290 | 10877492 | + |
| gRNA162 | TAR1054 | 348 | 10877550 | − |
| gRNA163 | TAR998 | −48 | 10877154 | + |
| gRNA164 | TAR271 | −2 | 10877200 | + |
| gRNA165 | TAR272 | 48 | 10877250 | + |
| gRNA166 | TAR1012 | 76 | 10877278 | + |
| gRNA167 | TAR1017 | 120 | 10877322 | − |
| gRNA168 | TAR1024 | 159 | 10877361 | − |
| gRNA169 | TAR1031 | 238 | 10877440 | − |
| gRNA170 | TAR1039 | 255 | 10877457 | − |
| gRNA171 | TAR1047 | 294 | 10877496 | + |
| gRNA172 | TAR1055 | 350 | 10877552 | + |
| gRNA173 | TAR1062 | 397 | 10877599 | − |
| gRNA174 | TAR1070 | 469 | 10877671 | − |
| gRNA175 | TAR1078 | 502 | 10877704 | − |
| gRNA176 | TAR1086 | 549 | 10877751 | − |
| gRNA177 | TAR1094 | 585 | 10877787 | + |
| gRNA178 | TAR1102 | 625 | 10877827 | + |
| gRNA179 | TAR1110 | 662 | 10877864 | − |
| gRNA180 | TAR1118 | 683 | 10877885 | + |
| gRNA181 | TAR1126 | 738 | 10877940 | − |
| gRNA182 | TAR1134 | 1772 | 10878974 | + |
| gRNA183 | TAR1142 | 1800 | 10879002 | + |
| gRNA184 | TAR1063 | 410 | 10877612 | − |
| gRNA185 | TAR1071 | 479 | 10877681 | − |
| gRNA186 | TAR1079 | 502 | 10877704 | + |
| gRNA187 | TAR1087 | 556 | 10877758 | − |
| gRNA188 | TAR1095 | 600 | 10877802 | − |
| gRNA189 | TAR1103 | 654 | 10877856 | − |
| gRNA190 | TAR1111 | 669 | 10877871 | + |
| gRNA191 | TAR1119 | 684 | 10877886 | + |
| gRNA192 | TAR1127 | 743 | 10877945 | + |
| gRNA193 | TAR1135 | 1773 | 10878975 | − |
| gRNA194 | TAR1143 | 1801 | 10879003 | + |
| gRNA195 | TAR1064 | 416 | 10877618 | + |
| gRNA196 | TAR1072 | 480 | 10877682 | − |
| gRNA197 | TAR1080 | 503 | 10877705 | − |
| gRNA198 | TAR1088 | 557 | 10877759 | + |
| gRNA199 | TAR1096 | 612 | 10877814 | + |
| gRNA200 | TAR1104 | 654 | 10877856 | + |
| gRNA201 | TAR1112 | 670 | 10877872 | + |
| gRNA202 | TAR1120 | 693 | 10877895 | + |
| gRNA203 | TAR1128 | 745 | 10877947 | − |
| gRNA204 | TAR1136 | 1775 | 10878977 | + |
| gRNA205 | TAR1144 | 1804 | 10879006 | − |
| gRNA206 | TAR1065 | 417 | 10877619 | + |
| gRNA207 | TAR1073 | 487 | 10877689 | + |
| gRNA208 | TAR1081 | 514 | 10877716 | + |
| gRNA209 | TAR1089 | 558 | 10877760 | + |
| gRNA210 | TAR1097 | 613 | 10877815 | + |
| gRNA211 | TAR1105 | 655 | 10877857 | − |
| gRNA212 | TAR1113 | 675 | 10877877 | + |
| gRNA213 | TAR1121 | 696 | 10877898 | + |
| gRNA214 | TAR1129 | 1731 | 10878933 | + |
| gRNA215 | TAR1137 | 1776 | 10878978 | + |
| gRNA216 | TAR1145 | 1818 | 10879020 | + |
| gRNA217 | TAR1066 | 446 | 10877648 | + |
| gRNA218 | TAR1074 | 488 | 10877690 | + |
| gRNA219 | TAR1082 | 518 | 10877720 | + |
| gRNA220 | TAR1090 | 561 | 10877763 | − |
| gRNA221 | TAR1098 | 614 | 10877816 | + |
| gRNA222 | TAR1106 | 658 | 10877860 | − |
| gRNA223 | TAR1114 | 676 | 10877878 | + |
| gRNA224 | TAR1122 | 697 | 10877899 | + |
| gRNA225 | TAR1130 | 1747 | 10878949 | − |
| gRNA226 | TAR1138 | 1779 | 10878981 | − |
| gRNA227 | TAR1146 | 1822 | 10879024 | + |
| gRNA228 | TAR1067 | 451 | 10877653 | + |
| gRNA229 | TAR1075 | 499 | 10877701 | − |
| gRNA230 | TAR1083 | 519 | 10877721 | + |
| gRNA231 | TAR1091 | 566 | 10877768 | − |
| gRNA232 | TAR1099 | 621 | 10877823 | − |
| gRNA233 | TAR1107 | 659 | 10877861 | − |
| gRNA234 | TAR1115 | 677 | 10877879 | + |
| gRNA235 | TAR1123 | 720 | 10877922 | + |
| gRNA236 | TAR1131 | 1756 | 10878958 | + |
| gRNA237 | TAR1139 | 1780 | 10878982 | − |
| gRNA238 | TAR1147 | 1826 | 10879028 | − |
| gRNA239 | TAR1068 | 456 | 10877658 | + |
| gRNA240 | TAR1076 | 500 | 10877702 | + |
| gRNA241 | TAR1084 | 527 | 10877729 | − |
| gRNA242 | TAR1092 | 571 | 10877773 | + |
| gRNA243 | TAR1100 | 622 | 10877824 | − |
| gRNA244 | TAR1108 | 660 | 10877862 | − |
| gRNA245 | TAR1116 | 681 | 10877883 | + |
| gRNA246 | TAR1124 | 726 | 10877928 | + |
| gRNA247 | TAR1132 | 1768 | 10878970 | + |
| gRNA248 | TAR1140 | 1781 | 10878983 | + |
| gRNA249 | TAR1069 | 468 | 10877670 | + |
| gRNA250 | TAR1077 | 501 | 10877703 | + |
| gRNA251 | TAR1085 | 543 | 10877745 | − |
| gRNA252 | TAR1093 | 584 | 10877786 | − |
| gRNA253 | TAR1101 | 622 | 10877824 | + |
| gRNA254 | TAR1109 | 661 | 10877863 | − |
| gRNA255 | TAR1117 | 682 | 10877884 | + |
| gRNA256 | TAR1125 | 735 | 10877937 | + |
| gRNA257 | TAR1133 | 1771 | 10878973 | + |
| gRNA258 | TAR1141 | 1781 | 10878983 | − |
| gRNA259 | TAR1148 | 1827 | 10879029 | − |
| gRNA260 | TAR1156 | 1991 | 10879193 | − |
| gRNA261 | TAR1164 | 2025 | 10879227 | + |
| gRNA262 | TAR1149 | 1848 | 10879050 | − |
| gRNA263 | TAR1157 | 1996 | 10879198 | + |
| gRNA264 | TAR1165 | 2029 | 10879231 | − |
| gRNA265 | TAR1150 | 1852 | 10879054 | + |
| gRNA266 | TAR1158 | 1997 | 10879199 | + |
| gRNA267 | TAR1166 | 2036 | 10879238 | − |
| gRNA268 | TAR1151 | 1856 | 10879058 | + |
| gRNA269 | TAR1159 | 2004 | 10879206 | + |
| gRNA270 | TAR1167 | 2037 | 10879239 | − |
| gRNA271 | TAR1152 | 1857 | 10879059 | − |
| gRNA272 | TAR1160 | 2010 | 10879212 | + |
| gRNA273 | TAR1168 | 2047 | 10879249 | + |
| gRNA274 | TAR1153 | 1857 | 10879059 | + |
| gRNA275 | TAR1161 | 2011 | 10879213 | + |
| gRNA276 | TAR1154 | 1973 | 10879175 | − |
| gRNA277 | TAR1162 | 2018 | 10879220 | + |
| gRNA278 | TAR1155 | 1983 | 10879185 | − |
| gRNA279 | TAR1163 | 2022 | 10879224 | − |

### Example 3: Selection of ZF Target Sites and Design of ZFPs

A library of two-finger ZFPs (2F units), each recognizing six bp DNA sites, was used to design larger six-finger ZFP arrays targeting 18 bp DNA binding sites. The source of the 2F units was a set of three-finger zinc finger proteins that had been selected to bind specific target sites using a bacterial-2-hybrid (B2H) selection system (Hurt et al., PNAS (2003) 100:12271-6; Maeder et al., Mol Cell (2008) 31 (2): 294-301). A list of targetable DNA sites was created by generating all possible triplet combinations of 6 bp binding sites represented in the library and allowing either 0 or 1 bp between the 6 bp target sites. To identify ZF target sites within human CIITA, the sequence within 1 kb of TSS1 (human (GRCh38)) was interrogated against this list.

For each identified ZF target site, multiple ZF proteins could be designed. Design of the six recognition helices used to generate the full proteins was performed by selecting 2F units and taking into account factors such as known binding preferences of zinc finger proteins, the frequency with which amino acids in positions—1, 2, 3 and 6 had been selected in the B2H selection system to bind the desired target base, avoidance of amino acids in positions—1, 2, 3 and 6 that had been selected to bind multiple different bases in the B2H, and maintenance of context dependencies by matching flanking bases where possible. The full ZF sequence is derived from the naturally occurring Zif268 protein and selected recognition helices were maintained in the sequence context in which they were selected in the B2H (either fingers 1-2 or fingers 2-3 from Zif268).

2F units were joined by the linker TGSQKP (SEQ ID NO: 651) where 6 bp binding sites were contiguous and by the linker TGGGGSQKP (SEQ ID NO: 652) where 1 bp separated the 6 bp binding sites. A final set of 237 ZFPs targeting 55 distinct DNA binding sites within 1 kb of CIITA TSS1 (chr16: 10877202) with no other exact matches to the genome (GRCh38) were selected for the primary screen (Table 1).

### Example 4: Guide RNA Screening in Primary Human T Cells

This Example describes a study in which gRNAs were screened for their efficacy in targeting CIITA in primary human T cells.

T cells were isolated from human leukapheresis product (StemCell Technologies, Cat. No. 70500) using the EasySep™ Human T cell Isolation Kit (StemCell Technologies, Cat. No. 17951). Prior to nucleofection, T cells were thawed, washed, and stimulated using Dynabeads Human T-Activator CD3/CD28 for T Cell Expansion and Activation (Thermo Fisher, Cat. No. 11131D) at a 3:1 bead-to-cell number ratio for approximately 72 hours at 37° C. with 5% CO₂ in T cell medium (X-VIVO15 media (Lonza®, Cat. No. BEBP04-744Q) supplemented with 5% Human AB serum (Gemini Bio-Product, Cat. No. 100-512), 2 mM L-alanyl-L-glutamine, 100 U/ml IL-2, 10 ng/ml IL-7, and 10 ng/ml IL-15. Beads were then magnetically removed from the culture and T cells were cultured in fresh complete T cell medium for approximately 24 hours. T cells were then nucleofected with 5 μg CRISPR-off mRNA (TriLink) plus 2.5 μg sgRNA (IDT) at 2E5 cells/well using the P3 Primary Cell 96-well Nucleofector Kit (Lonza®, Cat. No. V4SP-3960) and the Amaxa 4D Nucleofector® (Lonza) with pulse code EO115.

After nucleofection, T cells were resuspended in complete T cell medium and maintained by replacement of media and passages as necessary twice weekly. Cells were restimulated with ImmunoCult™ Human CD3/CD28 T Cell Activator (StemCell Technologies, Cat. No. 10991) on day 13 post-nucleofection.

Cell surface HLA-DR protein expression on live T cells was assessed by flow cytometry at days 6 and 20 post-nucleofection. No mRNA, CRISPR-off mRNA plus non-CIITA targeting sgRNA, CRISPR-off mRNA with no gRNA, WT Cas9 mRNA plus exon-targeting sgRNA, stain only (no mRNA or gRNA), isotype (no mRNA or gRNA), and no-stain (no mRNA or gRNA) controls were also run on each screening plate.

### HLA-DR Flow Cytometry

On days 6 and 20 post-nucleofection, an aliquot of T cells was assessed by flow cytometric staining while a remaining split of cells continued to be maintained in culture. The cells to be stained had media aspirated, were washed once with PBS containing 2% FBS, and were stained with PE-conjugated anti-human HLA-DR Antibody (BioLegend, Cat. No. 327008) at a 1:300 dilution and Zombie Violet™ Fixable Viability Dye (BioLegend, Cat. No. 423113), previously prepared according to manufacturer's recommendations, at a 1:1000 dilution in PBS with 2% FBS at 4° C. for 20 minutes. The stained cells were washed and incubated in Fixation Buffer (BioLegend, Cat. No. 420801) for 20 minutes. The cells were then washed prior to acquisition on an Agilent Novocyte Penteon flow cytometer, which could collect up to 20,000 live-cell events per well. Screening conditions were compared to negative (CRISPR-Off mRNA with no sgRNA) control expression levels to assess % silencing.

### Results

The relative HLA-DR expression levels measured in two separate experiments at days 6 and 20 in cells nucleofected with one of the 279 tested gRNAs, and the corresponding genomic distance of each gRNA relative to the CIITA TSS1, are shown in FIGS. 1A-1D, and in Table 9. The top performing CIITA gRNAs are designated in bold text in Table 9.

Silencing of the CIITA gene, with only 30-40% HLA-DR expression relative to the no-gRNA negative control, was observed after treatment with a number of gRNA candidates. The sequences and efficacies of the tested gRNAs are shown in Table 9. The relative efficiency of CIITA silencing is measured by reduction of HLA-DR cell surface expression.

Relative HLA-DR expression represents the averaged frequency of HLA-DR positive cells of the treated samples expressed as a percent of the average across no gRNA negative control conditions. The top 40 gRNAs with the most CIITA silencing, as evidenced by low HLA-DR cell surface protein expression, were selected for secondary screening.

TABLE 9
Target Sequences of Top Performing gRNAs Targeting CIITA

| | | | Distance | | % HLA−DR expression relative | | | |
|---|---|---|---|---|---|---|---|---|
| | | | from | Chr. | to no gRNA control | | | |
| gRNA | | | canonical | 16 | Experiment 1 | | Experiment 2 | |
| No. | DNA Target Sequence | SEQ | TSS | Strand | Day 6 | Day 20 | Day 6 | Day 20 |
| gRNA | GAGTGATCATATATTA | 755 | −724 | + | 86.0 | 95.75 | | |
| 001 | GAAG | | | | | | | |
| gRNA | GTCTCTGCCCATCCTC | 756 | −657 | − | 72.6 | 80.19 | | |
| 002 | CTGG | | | | | | | |
| gRNA | ATTATTTAGCATCACT | 757 | −597 | + | 96.8 | 112.15 | | |
| 003 | TTGG | | | | | | | |
| gRNA | GACTACAAGGGTACCA | 758 | −557 | + | 80.7 | 88.34 | | |
| 004 | TATT | | | | | | | |
| gRNA | TATGATCTCCAGAGAA | 759 | −464 | + | 93.4 | 106.68 | | |
| 005 | ATTC | | | | | | | |
| gRNA | TGTGGGAGTAGGCATG | 760 | −432 | + | 54.9 | 97.44 | | |
| 006 | GTAG | | | | | | | |
| gRNA | CTTGTCTTGAGGCATC | 761 | −371 | + | 44.0 | 112.27 | 10.26 | 67.92 |
| 007 | TGGG | | | | | | | |
| gRNA | ATCTGCCACCTTCTGC | 762 | −330 | − | 57.8 | 85.04 | | |
| 008 | AGGA | | | | | | | |
| gRNA | TTGCACAGAAAGTCTG | 763 | −240 | − | 42.2 | 80.55 | 7.21 | 44.69 |
| 009 | TTGG | | | | | | | |
| gRNA | TGAAATTAATTTCAGA | 764 | −179 | + | 91.8 | 100.35 | | |
| 010 | GGTG | | | | | | | |
| gRNA | AGGGAGTGTGGTAAAA | 765 | −146 | + | 52.5 | 72.85 | | |
| 011 | TTAG | | | | | | | |
| gRNA | TCAAGGAGGAAGTCTC | 766 | −682 | − | 74.4 | 86.75 | | |
| 012 | AGCT | | | | | | | |
| gRNA | CCAGGAGGATGGGCAG | 767 | −654 | + | 83.1 | 84.83 | | |
| 013 | AGAC | | | | | | | |
| gRNA | TTATTTAGCATCACTT | 768 | −596 | + | 95.1 | 111.73 | | |
| 014 | TGGC | | | | | | | |
| gRNA | ACTACAAGGGTACCAT | 769 | −556 | + | 85.2 | 98.33 | | |
| 015 | ATTT | | | | | | | |
| gRNA | ATGATCTCCAGAGAAA | 770 | −463 | + | 72.2 | 93.66 | | |
| 016 | TTCA | | | | | | | |
| gRNA | ATCTAAGAAGTCCCCA | 771 | −401 | + | 55.4 | 78.6 | | |
| 017 | GCAG | | | | | | | |
| gRNA | GTCTTGAGGCATCTGG | 772 | −368 | + | 51.0 | 89.65 | | |
| 018 | GCGG | | | | | | | |
| gRNA | CAATATCTGCCACCTT | 773 | −326 | − | 83.9 | 95.24 | | |
| 019 | CTGC | | | | | | | |
| gRNA | GTTGCACAGAAAGTCT | 774 | −239 | − | 47.1 | 80.15 | | |
| 020 | GTTG | | | | | | | |
| gRNA | GAAATTAATTTCAGAG | 775 | −178 | + | 81.5 | 95.19 | | |
| 021 | GTGT | | | | | | | |
| gRNA | GGGAGTGTGGTAAAAT | 776 | −144 | + | 58.4 | 71.76 | | |
| 022 | TAGA | | | | | | | |
| gRNA | ACTTCCTCCTTGATAC | 777 | −672 | + | 79.2 | 84.27 | | |
| 023 | CACC | | | | | | | |
| gRNA | CCAGTCTCTGCCCATC | 778 | −654 | − | 75.8 | 90.37 | | |
| 024 | CTCC | | | | | | | |
| gRNA | CCCCAAACCATCTGAC | 779 | −570 | + | 90.2 | 100.5 | | |
| 025 | TACA | | | | | | | |
| gRNA | AAAGAGTGTTAACCCA | 780 | −544 | − | 88.6 | 98.13 | | |
| 026 | AATA | | | | | | | |
| gRNA | CAAATTGCCCTGAATT | 781 | −456 | − | 93.8 | 100.62 | | |
| 027 | TCTC | | | | | | | |
| gRNA | CAAGCTGAGAGCCTCT | 782 | −390 | − | 60.1 | 97.15 | | |
| 028 | GCTG | | | | | | | |
| gRNA | TCTTGAGGCATCTGGG | 783 | −367 | + | 53.2 | 84.95 | | |
| 029 | CGGA | | | | | | | |
| gRNA | CTGCAGAAGGTGGCAG | 784 | −325 | + | 49.3 | 93.33 | | |
| 030 | ATAT | | | | | | | |
| gRNA | AGTTGCACAGAAAGTC | 785 | −238 | − | 49.3 | 91.31 | | |
| 031 | TGTT | | | | | | | |
| gRNA | AAATTAATTTCAGAGG | 786 | −177 | + | 97.6 | 100 | | |
| 032 | TGTG | | | | | | | |
| gRNA | GAAACAGAAATCTGAC | 787 | −116 | + | 76.7 | 87.06 | | |
| 033 | CGCT | | | | | | | |
| gRNA | TCCTCCTTGATACCAC | 788 | −669 | + | 73.5 | 80.59 | | |
| 034 | CAGG | | | | | | | |
| gRNA | AGACACTAACTTTCTC | 789 | −628 | + | 80.1 | 98.66 | | |
| 035 | CCTA | | | | | | | |
| gRNA | CCTTGTAGTCAGATGG | 790 | −570 | − | 98.8 | 87.21 | | |
| 036 | TTTG | | | | | | | |
| gRNA | TATTTGGGTTAACACT | 791 | −541 | + | 92.8 | 89.42 | | |
| 037 | CITT | | | | | | | |
| gRNA | CAGAGAAATTCAGGGC | 792 | −455 | + | 90.4 | 105.45 | | |
| 038 | AATT | | | | | | | |
| gRNA | ACAAGCTGAGAGCCTC | 793 | −389 | − | 69.8 | 78.77 | | |
| 039 | TGCT | | | | | | | |
| gRNA | TGGGCGGAGGGCTATG | 794 | −355 | + | 75.2 | 76.92 | | |
| 040 | ATAC | | | | | | | |
| gRNA | AGGTGGCAGATATTGG | 795 | −318 | + | 66.8 | 91.73 | | |
| 041 | CAGC | | | | | | | |
| gRNA | AAGTTGCACAGAAAGT | 796 | −237 | − | 60.6 | 87.47 | | |
| 042 | CTGT | | | | | | | |
| gRNA | TTAATTTCAGAGGTGT | 797 | −174 | + | 68.8 | 95.81 | | |
| 043 | GGGG | | | | | | | |
| gRNA | AAACAGAAATCTGACC | 798 | −115 | + | 75.7 | 82.28 | | |
| 044 | GCTT | | | | | | | |
| gRNA | TCCTCCTGGTGGTATC | 799 | −668 | − | 68.8 | 75.95 | | |
| 045 | AAGG | | | | | | | |
| gRNA | GACACTAACTTTCTCC | 800 | −627 | + | 73.4 | 89.68 | | |
| 046 | CTAT | | | | | | | |
| gRNA | CCCAAACCATCTGACT | 801 | −569 | + | 69.4 | 99.45 | | |
| 047 | ACAA | | | | | | | |
| gRNA | TGTCTTGGGATGAAAA | 802 | −496 | + | 85.7 | 92.67 | | |
| 048 | TGAC | | | | | | | |
| gRNA | AAATTCAGGGCAATTT | 803 | −450 | + | 73.6 | 83.78 | | |
| 049 | GGTG | | | | | | | |
| gRNA | GACAAGCTGAGAGCCT | 804 | −388 | − | 68.7 | 83.63 | | |
| 050 | CTGC | | | | | | | |
| gRNA | TACTGGCCCCATCCTG | 805 | −338 | + | 73.9 | 99.97 | | |
| 051 | CAGA | | | | | | | |
| gRNA | ATTGGCAGCTGGCACC | 806 | −307 | + | 51.2 | 80.76 | | |
| 052 | AGTG | | | | | | | |
| gRNA | ACCAAATTCAGTCCAC | 807 | −205 | + | 54.3 | 68.08 | | |
| 053 | AGTA | | | | | | | |
| gRNA | TAATTTCAGAGGTGTG | 808 | −173 | + | 97.3 | 92.89 | | |
| 054 | GGGA | | | | | | | |
| gRNA | AACAGAAATCTGACCG | 809 | −114 | + | 92.0 | 99 | | |
| 055 | CTTG | | | | | | | |
| gRNA | CCATCCTCCTGGTGGT | 810 | −665 | − | 67.7 | 78.91 | | |
| 056 | ATCA | | | | | | | |
| gRNA | AAATAATACTGACTCC | 811 | −613 | − | 75.2 | 80.93 | | |
| 057 | CATA | | | | | | | |
| gRNA | CCCTTGTAGTCAGATG | 812 | −569 | − | 86.8 | 89.19 | | |
| 058 | GTTT | | | | | | | |
| gRNA | CTTGGGATGAAAATGA | 813 | −493 | + | 89.1 | 91.02 | | |
| 059 | CAGG | | | | | | | |
| gRNA | AATTCAGGGCAATTTG | 814 | −449 | + | 86.5 | 94.51 | | |
| 060 | GTGT | | | | | | | |
| gRNA | GAGGCTCTCAGCTTGT | 815 | −382 | + | 56.7 | 88.6 | | |
| 061 | CTTG | | | | | | | |
| gRNA | TGGCCCCATCCTGCAG | 816 | −335 | + | 56.8 | 101.57 | | |
| 062 | AAGG | | | | | | | |
| gRNA | TGATCACAATGGAACC | 817 | −293 | − | 60.0 | 80.21 | | |
| 063 | GCAC | | | | | | | |
| gRNA | TCCTTACTGTGGACTG | 818 | −200 | − | 45.0 | 86.02 | 17.13 | 80.40 |
| 064 | AATT | | | | | | | |
| gRNA | AGAGGTGTGGGGAGGG | 819 | −166 | + | 63.4 | 79.3 | | |
| 065 | CTTA | | | | | | | |
| gRNA | ACCGCTTGGGGCCACC | 820 | −102 | + | 34.6 | 65.75 | 17.16 | 56.68 |
| 066 | TTGC | | | | | | | |
| gRNA | CCTTGATACCACCAGG | 821 | −665 | + | 66.9 | 101.37 | | |
| 067 | AGGA | | | | | | | |
| gRNA | TAAATAATACTGACTC | 822 | −612 | − | 84.0 | 87.64 | | |
| 068 | CCAT | | | | | | | |
| gRNA | ACCCTTGTAGTCAGAT | 823 | −568 | − | 88.9 | 88.54 | | |
| 069 | GGTT | | | | | | | |
| gRNA | TTGGGATGAAAATGAC | 824 | −492 | + | 73.6 | 102.48 | | |
| 070 | AGGT | | | | | | | |
| gRNA | GGGCAATTTGGTGTGG | 825 | −443 | + | 54.3 | 85.08 | | |
| 071 | GAGT | | | | | | | |
| gRNA | TCAGCTTGTCTTGAGG | 826 | −375 | + | 78.1 | 84.7 | | |
| 072 | CATC | | | | | | | |
| gRNA | CTGCCACCTTCTGCAG | 827 | −332 | − | 52.3 | 89.9 | | |
| 073 | GATG | | | | | | | |
| gRNA | TTCAGAAATGATGATC | 828 | −282 | − | 54.1 | 79.59 | | |
| 074 | ACAA | | | | | | | |
| gRNA | TTAATTTCACTTCCTT | 829 | −193 | − | 67.7 | 86.18 | | |
| 075 | ACTG | | | | | | | |
| gRNA | GAGGTGTGGGGAGGGC | 830 | −165 | + | 43.5 | 90.38 | 25.09 | 62.17 |
| 076 | TTAA | | | | | | | |
| gRNA | CTTGATACCACCAGGA | 831 | −664 | + | 78.7 | 81.83 | | |
| 077 | GGAT | | | | | | | |
| gRNA | AGTATTATTTAGCATC | 832 | −600 | + | 81.7 | 97.37 | | |
| 078 | ACTT | | | | | | | |
| gRNA | ATGGTACCCTTGTAGT | 833 | −563 | − | 79.1 | 98.11 | | |
| 079 | CAGA | | | | | | | |
| gRNA | ATTTCTCTGGAGATCA | 834 | −469 | − | 72.8 | 92.64 | | |
| 080 | TAAG | | | | | | | |
| gRNA | ATTTGGTGTGGGAGTA | 835 | −438 | + | 67.5 | 93.02 | | |
| 081 | GGCA | | | | | | | |
| gRNA | CAGCTTGTCTTGAGGC | 836 | −374 | + | 53.7 | 94.47 | | |
| 082 | ATCT | | | | | | | |
| gRNA | TCTGCCACCTTCTGCA | 837 | −331 | − | 51.1 | 92.48 | | |
| 083 | GGAT | | | | | | | |
| gRNA | TCTGAACGTCAGACTG | 838 | −265 | + | 41.1 | 74.61 | 16.48 | 65.17 |
| 084 | TTGA | | | | | | | |
| gRNA | GGAAGTGAAATTAATT | 839 | −184 | + | 49.0 | 87.82 | | |
| 085 | TCAG | | | | | | | |
| gRNA | GGGGAGGGCTTAAGGG | 840 | −158 | + | 42.0 | 87.18 | 18.60 | 38.45 |
| 086 | AGTG | | | | | | | |
| gRNA | CCCTGCAAGGTGGCCC | 841 | −97 | − | 60.5 | 61.68 | | |
| 087 | CAAG | | | | | | | |
| gRNA | TGCATGTTGGCTTAGC | 842 | −45 | + | 65.9 | 68.37 | | |
| 088 | TTGG | | | | | | | |
| gRNA | GAGGCTGTGTGCTTCT | 843 | 18 | + | 68.8 | 84.2 | | |
| 089 | GAG | | | | | | | |
| gRNA | GGCATCCTTGGGGAAG | 844 | 49 | + | 64.4 | 100.74 | | |
| 090 | CTGA | | | | | | | |
| gRNA | AGGCAGCAGCTCCCGG | 845 | 87 | − | 97.5 | 82.43 | | |
| 091 | AGTC | | | | | | | |
| gRNA | TGCCTGGCTCCACGCC | 846 | 134 | + | 103.6 | 91.47 | | |
| 092 | CTGC | | | | | | | |
| gRNA | GTCAGAGCCCCAAGGT | 847 | 166 | + | 89.5 | 107.3 | | |
| 093 | AAAA | | | | | | | |
| gRNA | ACAGAGAAACCATTCT | 848 | 246 | + | 116.5 | 84.55 | | |
| 094 | GAAT | | | | | | | |
| gRNA | CTGAATTGGGGATGGG | 849 | 260 | + | 91.0 | 126.74 | | |
| 095 | GGTG | | | | | | | |
| gRNA | GTCCTGCTGGGGCAGG | 850 | 301 | + | 95.0 | 133.82 | | |
| 096 | CCAT | | | | | | | |
| gRNA | GACCTGAAGTCTCCCT | 851 | 352 | − | 63.4 | 115.21 | | |
| 097 | CCGG | | | | | | | |
| gRNA | CCGCTTGGGGCCACCT | 852 | −101 | + | 45.9 | 60.9 | 10.56 | 38.95 |
| 098 | TGCA | | | | | | | |
| gRNA | GCATGTTGGCTTAGCT | 853 | −44 | + | 35.5 | NA | 15.08 | 66.67 |
| 099 | TGGC | | | | | | | |
| gRNA | AGGCTGTGTGCTTCTG | 854 | 18 | + | 40.7 | NA | 10.32 | 37.20 |
| 100 | AGCT | | | | | | | |
| gRNA | TCGTGCCCTCAGCTTC | 855 | 54 | − | 33.9 | NA | 12.44 | 45.44 |
| 101 | CCCA | | | | | | | |
| gRNA | AGACTCCGGGAGCTGC | 856 | 89 | + | 57.9 | 63.13 | | |
| 102 | TGCC | | | | | | | |
| gRNA | GCCTGGCTCCACGCCC | 857 | 136 | + | 52.1 | NA | | |
| 103 | TGCT | | | | | | | |
| gRNA | GAGCCCCAAGGTAAAA | 858 | 170 | + | 106.5 | 108.86 | | |
| 104 | AGGC | | | | | | | |
| gRNA | CAGAGAAACCATTCTG | 859 | 247 | + | 98.6 | NA | 87.52 | 124.59 |
| 105 | AATT | | | | | | | |
| gRNA | ATTGGGGATGGGGGTG | 860 | 264 | + | 98.3 | NA | | |
| 106 | AGGA | | | | | | | |
| gRNA | TTCCAATGGCCTGCCC | 861 | 303 | − | 108.0 | NA | | |
| 107 | CAGC | | | | | | | |
| gRNA | GCTGACCTGAAGTCTC | 862 | 355 | − | 57.5 | 120.06 | | |
| 108 | CCTC | | | | | | | |
| gRNA | AAAAAACTCTCCCTGC | 863 | −91 | − | 99.9 | 93.72 | | |
| 109 | AAGG | | | | | | | |
| gRNA | TAGCTTGGCGGGCTCC | 864 | −33 | + | 31.5 | NA | 5.95 | 55.93 |
| 110 | CAAC | | | | | | | |
| gRNA | CTTCTGAGCTGGGCAT | 865 | 28 | + | 58.7 | NA | | |
| 111 | CCGA | | | | | | | |
| gRNA | TTGGGGAAGCTGAGGG | 866 | 56 | + | NA | NA | | |
| 112 | CACG | | | | | | | |
| gRNA | TCCGGGAGCTGCTGCC | 867 | 93 | + | 76.6 | NA | | |
| 113 | TGGC | | | | | | | |
| gRNA | ACCCAGCAGGGCGTGG | 868 | 136 | − | 85.1 | 76.55 | | |
| 114 | AGCC | | | | | | | |
| gRNA | AGCCCCAAGGTAAAAA | 869 | 171 | + | 55.8 | 134.02 | | |
| 115 | GGCC | | | | | | | |
| gRNA | AGAGAAACCATTCTGA | 870 | 248 | + | 85.1 | 99.78 | | |
| 116 | ATTG | | | | | | | |
| gRNA | TTGGGGATGGGGGTGA | 871 | 265 | + | 108.7 | NA | 54.02 | 132.08 |
| 117 | GGAT | | | | | | | |
| gRNA | AACTCTTTCACATCTT | 872 | 317 | − | 103.3 | NA | 70.77 | 114.11 |
| 118 | CCAA | | | | | | | |
| gRNA | AGGGAGACTTCAGGTC | 873 | 359 | + | 78.8 | 144.85 | | |
| 119 | AGCC | | | | | | | |
| gRNA | TCAAAAAAACTCTCCC | 874 | −88 | − | 93.7 | 74.59 | | |
| 120 | TGCA | | | | | | | |
| gRNA | GCGGGCTCCCAACTGG | 875 | −25 | + | 18.9 | NA | 12.27 | 43.70 |
| 121 | TGAC | | | | | | | |
| gRNA | TGGGCATCCGAAGGCA | 876 | 37 | + | 57.2 | NA | | |
| 122 | TCCT | | | | | | | |
| gRNA | GGGAAGCTGAGGGCAC | 877 | 59 | + | 23.9 | NA | 27.97 | 47.44 |
| 123 | GAGG | | | | | | | |
| gRNA | CCCAGCCAGGCAGCAG | 878 | 94 | − | 79.8 | 118.71 | | |
| 124 | CTCC | | | | | | | |
| gRNA | AGGTAGGACCCAGCAG | 879 | 143 | − | 44.2 | 103.34 | 22.70 | 48.19 |
| 125 | GGCG | | | | | | | |
| gRNA | GCTAAATTAAGATGCT | 880 | 189 | − | 97.7 | NA | | |
| 126 | TTCC | | | | | | | |
| gRNA | AAACCATTCTGAATTG | 881 | 252 | + | 93.4 | NA | | |
| 127 | GGGA | | | | | | | |
| gRNA | ATGGGGGTGAGGATGG | 882 | 271 | + | 73.5 | NA | | |
| 128 | GAAC | | | | | | | |
| gRNA | AGTTGTCTATTTCCTT | 883 | 336 | + | 65.9 | NA | | |
| 129 | CCAC | | | | | | | |
| gRNA | CTTCAGGTCAGCCAGG | 884 | 366 | + | 52.7 | NA | | |
| 130 | TGTC | | | | | | | |
| gRNA | ACATGCAAAGAAACAA | 885 | −61 | − | 77.1 | 88.53 | | |
| 131 | GTGA | | | | | | | |
| gRNA | TCACTAACCAGTCACC | 886 | −19 | − | 21.2 | NA | 9.13 | 52.43 |
| 132 | AGTT | | | | | | | |
| gRNA | GGGCATCCGAAGGCAT | 887 | 38 | + | 51.5 | 99.07 | | |
| 133 | CCTT | | | | | | | |
| gRNA | GGAAGCTGAGGGCACG | 888 | 60 | + | 110.9 | 86.43 | | |
| 134 | AGGA | | | | | | | |
| gRNA | CCGGGAGCTGCTGCCT | 889 | 94 | + | 96.4 | 102.57 | | |
| 135 | GGCT | | | | | | | |
| gRNA | CTGACAGGTAGGACCC | 890 | 148 | − | 55.1 | NA | | |
| 136 | AGCA | | | | | | | |
| gRNA | ATTTAGCGTGCAGTCT | 891 | 205 | + | 68.3 | NA | | |
| 137 | CAGC | | | | | | | |
| gRNA | AACCATTCTGAATTGG | 892 | 253 | + | 91.5 | 124.09 | | |
| 138 | GGAT | | | | | | | |
| gRNA | AACAGGAGTCTGTGTC | 893 | 288 | + | 78.8 | NA | | |
| 139 | CTGC | | | | | | | |
| gRNA | TGTCTATTTCCTTCCA | 894 | 339 | + | 82.1 | NA | | |
| 140 | CCGG | | | | | | | |
| gRNA | TGGTTCATACTCCAGA | 895 | 377 | − | 88.4 | 148.83 | | |
| 141 | CACC | | | | | | | |
| gRNA | AACATGCAAAGAAACA | 896 | −60 | − | 76.9 | 67.02 | | |
| 142 | AGTG | | | | | | | |
| gRNA | ATCACTAACCAGTCAC | 897 | −18 | − | 57.4 | NA | | |
| 143 | CAGT | | | | | | | |
| gRNA | GGCATCCGAAGGCATC | 898 | 39 | + | 33.4 | 99.96 | 11.83 | 47.69 |
| 144 | CTTG | | | | | | | |
| gRNA | GAAGCTGAGGGCACGA | 899 | 61 | + | 41.9 | NA | | |
| 145 | GGAG | | | | | | | |
| gRNA | ATTGTGTAGGAATCCC | 900 | 107 | − | 73.8 | NA | | |
| 146 | AGCC | | | | | | | |
| gRNA | TCTGACAGGTAGGACC | 901 | 149 | − | 73.4 | NA | | |
| 147 | CAGC | | | | | | | |
| gRNA | CTCTGTTTATCTGGAA | 902 | 229 | − | 79.1 | NA | | |
| 148 | TGGC | | | | | | | |
| gRNA | ACCATTCTGAATTGGG | 903 | 254 | + | 82.7 | NA | | |
| 149 | GATG | | | | | | | |
| gRNA | ACAGGAGTCTGTGTCC | 904 | 289 | + | 95.5 | NA | | |
| 150 | TGCT | | | | | | | |
| gRNA | GTCTATTTCCTTCCAC | 905 | 340 | + | 102.0 | NA | 55.38 | 75.91 |
| 151 | CGGA | | | | | | | |
| gRNA | AACCATGTATCAGCAC | 906 | 395 | + | 95.7 | 120.06 | | |
| 152 | CGAA | | | | | | | |
| gRNA | TCACTTGTTTCTTTGC | 907 | −58 | + | 102.0 | 75.29 | | |
| 153 | ATGT | | | | | | | |
| gRNA | CTGGTGACTGGTTAGT | 908 | −14 | + | 57.7 | 100.62 | | |
| 154 | GATG | | | | | | | |
| gRNA | AGCTTCCCCAAGGATG | 909 | 44 | − | 56.2 | NA | | |
| 155 | CCTT | | | | | | | |
| gRNA | GAGGAGGGGCTGCCAG | 910 | 76 | + | 52.6 | NA | | |
| 156 | ACTC | | | | | | | |
| gRNA | TTCCTACACAATGCGT | 911 | 118 | + | 87.5 | NA | | |
| 157 | TGCC | | | | | | | |
| gRNA | TCCTACCTGTCAGAGC | 912 | 158 | + | 67.9 | NA | | |
| 158 | CCCA | | | | | | | |
| gRNA | GTTTCTCTGTTTATCT | 913 | 237 | − | 106.1 | NA | 72.82 | 123.10 |
| 159 | GGAA | | | | | | | |
| gRNA | CCATTCTGAATTGGGG | 914 | 255 | + | 138.1 | NA | 73.50 | 118.60 |
| 160 | ATGG | | | | | | | |
| gRNA | CAGGAGTCTGTGTCCT | 915 | 290 | + | 60.4 | NA | | |
| 161 | GCTG | | | | | | | |
| gRNA | TGAAGTCTCCCTCCGG | 916 | 348 | − | 51.4 | 138.08 | | |
| 162 | TGGA | | | | | | | |
| gRNA | CTTTGCATGTTGGCTT | 917 | −48 | + | 54.5 | 81.11 | | |
| 163 | AGCT | | | | | | | |
| gRNA | TAGTGATGAGGCTAGT | 918 | −2 | + | 43.4 | 69 | 10.84 | 42.20 |
| 164 | GATG | | | | | | | |
| gRNA | AGGCATCCTTGGGGAA | 919 | 48 | + | 64.0 | 87.8 | | |
| 165 | GCTG | | | | | | | |
| gRNA | AGGAGGGGCTGCCAGA | 920 | 76 | + | 68.6 | 121.44 | | |
| 166 | CTCC | | | | | | | |
| gRNA | AGCCAGGCAACGCATT | 921 | 120 | − | 79.9 | 104.57 | | |
| 167 | GTGT | | | | | | | |
| gRNA | ACCTTGGGGCTCTGAC | 922 | 159 | − | 87.4 | 100.67 | | |
| 168 | AGGT | | | | | | | |
| gRNA | GAATGGTTTCTCTGTT | 923 | 238 | − | 97.6 | 93.74 | | |
| 169 | TATC | | | | | | | |
| gRNA | CCCCCATCCCCAATTC | 924 | 255 | − | 82.7 | 92.74 | | |
| 170 | AGAA | | | | | | | |
| gRNA | AGTCTGTGTCCTGCTG | 925 | 294 | + | 78.4 | 139.17 | | |
| 171 | GGGC | | | | | | | |
| gRNA | TTCCACCGGAGGGAGA | 926 | 350 | + | 87.5 | 125.43 | | |
| 172 | CTTC | | | | | | | |
| gRNA | AACCTTTCGGTGCTGA | 927 | 397 | − | 73.0 | 112.28 | | |
| 173 | TACA | | | | | | | |
| gRNA | ACCTTGCTGTGGGCCG | 928 | 469 | − | 112.6 | 105.25 | | |
| 174 | AGCC | | | | | | | |
| gRNA | CCCCTCAGTATTTACC | 929 | 502 | − | 100.6 | 102.57 | | |
| 175 | CAAA | | | | | | | |
| gRNA | TCTGGGTATGGAGTGG | 930 | 549 | − | 77.6 | 119.88 | | |
| 176 | AGTC | | | | | | | |
| gRNA | CAAAATTGGAGTCAGC | 931 | 585 | + | 109.7 | 87.61 | | |
| 177 | CTTG | | | | | | | |
| gRNA | CAGCAGCTGGGGAGAG | 932 | 625 | + | 88.8 | 92.8 | | |
| 178 | CTGG | | | | | | | |
| gRNA | AAACAACACCATTAGG | 933 | 662 | − | 92.2 | 85.92 | | |
| 179 | AAAC | | | | | | | |
| gRNA | TTAAAAAGGGTCAGGG | 934 | 683 | + | 108.9 | 97.46 | | |
| 180 | GACG | | | | | | | |
| gRNA | CTGCCTTCAGAGCCGG | 935 | 738 | − | 99.4 | 96.14 | | |
| 181 | TGCC | | | | | | | |
| gRNA | GCCGCGGCCCCAGAGC | 936 | 1772 | + | 113.1 | 153.5 | | |
| 182 | TGGC | | | | | | | |
| gRNA | GAGGCCACCAGCAGCG | 937 | 1800 | + | 98.5 | 102.57 | | |
| 183 | CGCG | | | | | | | |
| gRNA | GTCTGACTTCTAGAAC | 938 | 410 | − | 92.6 | 117.1 | | |
| 184 | CTTT | | | | | | | |
| gRNA | AGGCGAGAAGACCTTG | 939 | 479 | − | 84.1 | NA | | |
| 185 | CTGT | | | | | | | |
| gRNA | CCCTTTGGGTAAATAC | 940 | 502 | + | NA | NA | | |
| 186 | TGAG | | | | | | | |
| gRNA | CTGCTTCTCTGGGTAT | 941 | 556 | − | NA | NA | | |
| 187 | GGAG | | | | | | | |
| gRNA | GGCTCAACAGCTACAC | 942 | 600 | − | NA | NA | | |
| 188 | CTCA | | | | | | | |
| gRNA | CCATTAGGAAACTGGG | 943 | 654 | − | NA | NA | | |
| 189 | GGGA | | | | | | | |
| gRNA | CCTAATGGTGTTGTTT | 944 | 669 | + | NA | NA | | |
| 190 | AAAA | | | | | | | |
| gRNA | TAAAAAGGGTCAGGGG | 945 | 684 | + | 60.0 | 91.06 | | |
| 191 | ACGG | | | | | | | |
| gRNA | CACCGGCTCTGAAGGC | 946 | 743 | + | 57.9 | 81.95 | | |
| 192 | AGCA | | | | | | | |
| gRNA | TCCCGCCAGCTCTGGG | 947 | 1773 | − | 80.9 | 105.25 | | |
| 193 | GCCG | | | | | | | |
| gRNA | AGGCCACCAGCAGCGC | 948 | 1801 | + | 103.2 | 77.64 | | |
| 194 | GCGC | | | | | | | |
| gRNA | GGTTCTAGAAGTCAGA | 949 | 416 | + | 88.5 | 103.94 | | |
| 195 | CTTT | | | | | | | |
| gRNA | GAGGCGAGAAGACCTT | 950 | 480 | − | NA | NA | | |
| 196 | GCTG | | | | | | | |
| gRNA | ACCCCTCAGTATTTAC | 951 | 503 | − | NA | NA | | |
| 197 | CCAA | | | | | | | |
| gRNA | CACTCCATACCCAGAG | 952 | 557 | + | NA | NA | | |
| 198 | AAGC | | | | | | | |
| gRNA | GCTGTTGAGCCCTCAG | 953 | 612 | + | NA | NA | | |
| 199 | CAGC | | | | | | | |
| gRNA | CCCTCCCCCCAGTTTC | 954 | 654 | + | 100.7 | NA | 52.99 | 101.12 |
| 200 | CTAA | | | | | | | |
| gRNA | CTAATGGTGTTGTTTA | 955 | 670 | + | 70.5 | NA | | |
| 201 | AAAA | | | | | | | |
| gRNA | TCAGGGGACGGGGGAA | 956 | 693 | + | 67.4 | NA | | |
| 202 | CAGA | | | | | | | |
| gRNA | TGCCATGCTGCCTTCA | 957 | 745 | − | 55.3 | 61.2 | | |
| 203 | GAGC | | | | | | | |
| gRNA | GCGGCCCCAGAGCTGG | 958 | 1775 | + | 75.4 | 116.67 | | |
| 204 | CGGG | | | | | | | |
| gRNA | GCTCCCGCGCGCGCTG | 959 | 1804 | − | 99.8 | 110.26 | | |
| 205 | CTGG | | | | | | | |
| gRNA | GTTCTAGAAGTCAGAC | 960 | 417 | + | 85.5 | 123.86 | | |
| 206 | TTTC | | | | | | | |
| gRNA | AAGGTCTTCTCGCCTC | 961 | 487 | + | 93.7 | NA | | |
| 207 | CCTT | | | | | | | |
| gRNA | ATACTGAGGGGTGCCT | 962 | 514 | + | 173.6 | NA | 53.33 | 93.88 |
| 208 | CTGC | | | | | | | |
| gRNA | ACTCCATACCCAGAGA | 963 | 558 | + | NA | NA | | |
| 209 | AGCA | | | | | | | |
| gRNA | CTGTTGAGCCCTCAGC | 964 | 613 | + | NA | NA | | |
| 210 | AGCT | | | | | | | |
| gRNA | ACCATTAGGAAACTGG | 965 | 655 | − | NA | NA | | |
| 211 | GGGG | | | | | | | |
| gRNA | GGTGTTGTTTAAAAAG | 966 | 675 | + | NA | NA | | |
| 212 | GGTC | | | | | | | |
| gRNA | GGGGACGGGGGAACAG | 967 | 696 | + | 47.8 | NA | 35.56 | 78.65 |
| 213 | ATGG | | | | | | | |
| gRNA | CGGGGAGGTAGGATGA | 968 | 1731 | + | 54.8 | 73.36 | | |
| 214 | CCAG | | | | | | | |
| gRNA | CGGCCCCAGAGCTGGC | 969 | 1776 | + | 64.6 | 119.98 | | |
| 215 | GGGA | | | | | | | |
| gRNA | CGCGGGAGCCCGGGGA | 970 | 1818 | + | 71.3 | 106.69 | | |
| 216 | ACAG | | | | | | | |
| gRNA | GTCACTAACTCTCAGC | 971 | 446 | + | 106.1 | 124.04 | | |
| 217 | ATGC | | | | | | | |
| gRNA | AGGTCTTCTCGCCTCC | 972 | 488 | + | 66.3 | 121.02 | | |
| 218 | CTTT | | | | | | | |
| gRNA | TGAGGGGTGCCTCTGC | 973 | 518 | + | NA | NA | | |
| 219 | AGGA | | | | | | | |
| gRNA | TTTCCCTGCTTCTCTG | 974 | 561 | − | 123.7 | NA | 22.05 | 75.16 |
| 220 | GGTA | | | | | | | |
| gRNA | TGTTGAGCCCTCAGCA | 975 | 614 | + | 32.2 | NA | 28.75 | 88.64 |
| 221 | GCTG | | | | | | | |
| gRNA | AACACCATTAGGAAAC | 976 | 658 | − | 120.8 | NA | 33.20 | 85.89 |
| 222 | TGGG | | | | | | | |
| gRNA | GTGTTGTTTAAAAAGG | 977 | 676 | + | 86.9 | 87.47 | | |
| 223 | GTCA | | | | | | | |
| gRNA | GGGACGGGGGAACAGA | 978 | 697 | + | 94.7 | 80.44 | | |
| 224 | TGGT | | | | | | | |
| gRNA | GTCTGTGGCAGCTCGT | 979 | 1747 | − | 98.8 | 68.34 | | |
| 225 | CCGC | | | | | | | |
| gRNA | TCTCCCTCCCGCCAGC | 980 | 1779 | − | 73.7 | 121.02 | | |
| 226 | TCTG | | | | | | | |
| gRNA | GGAGCCCGGGGAACAG | 981 | 1822 | + | 83.3 | 108.9 | | |
| 227 | CGGT | | | | | | | |
| gRNA | TAACTCTCAGCATGCT | 982 | 451 | + | 116.5 | 103.29 | | |
| 228 | GGCC | | | | | | | |
| gRNA | CTCAGTATTTACCCAA | 983 | 499 | − | 69.9 | 111.5 | | |
| 229 | AGGG | | | | | | | |
| gRNA | GAGGGGTGCCTCTGCA | 984 | 519 | + | 80.3 | 110.26 | | |
| 230 | GGAC | | | | | | | |
| gRNA | TTTGGTTTCCCTGCTT | 985 | 566 | − | NA | NA | | |
| 231 | CTCT | | | | | | | |
| gRNA | CAGCTCTCCCCAGCTG | 986 | 621 | − | 107.4 | 72.5 | | |
| 232 | CTGA | | | | | | | |
| gRNA | CAACACCATTAGGAAA | 987 | 659 | − | 99.2 | 93.2 | | |
| 233 | CTGG | | | | | | | |
| gRNA | TGTTGTTTAAAAAGGG | 988 | 677 | + | 92.7 | 82.73 | | |
| 234 | TCAG | | | | | | | |
| gRNA | AAGAGCACAGTGCAGA | 989 | 720 | + | 84.3 | 109.02 | | |
| 235 | CACC | | | | | | | |
| gRNA | GAGCTGCCACAGACTT | 990 | 1756 | + | 79.8 | 124.04 | | |
| 236 | GCCG | | | | | | | |
| gRNA | CTCTCCCTCCCGCCAG | 991 | 1780 | − | 83.8 | 111.5 | | |
| 237 | CTCT | | | | | | | |
| gRNA | GTCACCTACCGCTGTT | 992 | 1826 | − | 86.3 | 119.88 | | |
| 238 | CCCC | | | | | | | |
| gRNA | CTCAGCATGCTGGCCT | 993 | 456 | + | 110.3 | 126.03 | | |
| 239 | GGCT | | | | | | | |
| gRNA | CTCCCTTTGGGTAAAT | 994 | 500 | + | 89.2 | 119.39 | | |
| 240 | ACTG | | | | | | | |
| gRNA | GCAGAGGTCCCGTCCT | 995 | 527 | − | 91.0 | 106.69 | | |
| 241 | GCAG | | | | | | | |
| gRNA | AGAAGCAGGGAAACCA | 996 | 571 | + | 91.9 | 76.69 | | |
| 242 | AAAT | | | | | | | |
| gRNA | CCAGCTCTCCCCAGCT | 997 | 622 | − | 99.4 | 86.23 | | |
| 243 | GCTG | | | | | | | |
| gRNA | ACAACACCATTAGGAA | 998 | 660 | − | 97.6 | 87.11 | | |
| 244 | ACTG | | | | | | | |
| gRNA | GTTTAAAAAGGGTCAG | 999 | 681 | + | 88.5 | 90.75 | | |
| 245 | GGGA | | | | | | | |
| gRNA | ACAGTGCAGACACCTG | 1000 | 726 | + | 61.1 | 106.12 | | |
| 246 | GCAC | | | | | | | |
| gRNA | ACTTGCCGCGGCCCCA | 1001 | 1768 | + | 91.0 | 103.29 | | |
| 247 | GAGC | | | | | | | |
| gRNA | CCAGAGCTGGCGGGAG | 1002 | 1781 | + | 100.2 | 119.39 | | |
| 248 | GGAG | | | | | | | |
| gRNA | GCCTGGCTCGGCCCAC | 1003 | 468 | + | 98.4 | 153.5 | | |
| 249 | AGCA | | | | | | | |
| gRNA | TCCCTTTGGGTAAATA | 1004 | 501 | + | 96.3 | 137.23 | | |
| 250 | CTGA | | | | | | | |
| gRNA | TATGGAGTGGAGTCTG | 1005 | 543 | − | 82.2 | 108.9 | | |
| 251 | GCAG | | | | | | | |
| gRNA | CTCAAGGCTGACTCCA | 1006 | 584 | − | 94.6 | 66.26 | | |
| 252 | ATTT | | | | | | | |
| gRNA | CCTCAGCAGCTGGGGA | 1007 | 622 | + | 95.9 | 79.17 | | |
| 253 | GAGC | | | | | | | |
| gRNA | AACAACACCATTAGGA | 1008 | 661 | − | 104.6 | 91.67 | | |
| 254 | AACT | | | | | | | |
| gRNA | TTTAAAAAGGGTCAGG | 1009 | 682 | + | 104.7 | 92.58 | | |
| 255 | GGAC | | | | | | | |
| gRNA | ACACCTGGCACCGGCT | 1010 | 735 | + | 71.9 | 98.77 | | |
| 256 | CTGA | | | | | | | |
| gRNA | TGCCGCGGCCCCAGAG | 1011 | 1771 | + | 85.0 | 126.03 | | |
| 257 | CTGG | | | | | | | |
| gRNA | CCTCTCCCTCCCGCCA | 1012 | 1781 | − | 83.0 | 137.23 | | |
| 258 | GCTC | | | | | | | |
| gRNA | GGTCACCTACCGCTGT | 1013 | 1827 | − | 75.6 | 74.23 | | |
| 259 | TCCC | | | | | | | |
| gRNA | CAGAGTCGTTGCGGGG | 1014 | 1991 | − | 92.8 | 92.8 | | |
| 260 | ATGC | | | | | | | |
| gRNA | GGAGCCGGGAACCCGG | 1015 | 2025 | + | 100.7 | 87.47 | | |
| 261 | AGCT | | | | | | | |
| gRNA | AGGGGTTACAGAGGAG | 1016 | 1848 | − | 62.4 | 76.69 | | |
| 262 | ACTT | | | | | | | |
| gRNA | ATCCCCGCAACGACTC | 1017 | 1996 | + | 98.6 | 93.5 | | |
| 263 | TGCG | | | | | | | |
| gRNA | CAAGCCAAGCTCCGGG | 1018 | 2029 | − | 79.5 | 82.73 | | |
| 264 | TTCC | | | | | | | |
| gRNA | AGTCTCCTCTGTAACC | 1019 | 1852 | + | 57.1 | 66.26 | | |
| 265 | CCTA | | | | | | | |
| gRNA | TCCCCGCAACGACTCT | 1020 | 1997 | + | 100.5 | 93.97 | | |
| 266 | GCGC | | | | | | | |
| gRNA | GGCACAGCAAGCCAAG | 1021 | 2036 | − | 77.4 | 90.75 | | |
| 267 | CTCC | | | | | | | |
| gRNA | TCCTCTGTAACCCCTA | 1022 | 1856 | + | 69.6 | 87.61 | | |
| 268 | AGGT | | | | | | | |
| gRNA | AACGACTCTGCGCGGG | 1023 | 2004 | + | 92.7 | 93.2 | | |
| 269 | AACC | | | | | | | |
| gRNA | GGGCACAGCAAGCCAA | 1024 | 2037 | − | 77.7 | 92.58 | | |
| 270 | GCTC | | | | | | | |
| gRNA | CCCGACCTTAGGGGTT | 1025 | 1857 | − | 67.4 | 75.99 | | |
| 271 | ACAG | | | | | | | |
| gRNA | TCTGCGCGGGAACCAG | 1026 | 2010 | + | 90.6 | 87.11 | | |
| 272 | GAGC | | | | | | | |
| gRNA | GCTTGCTGTGCCCAGA | 1027 | 2047 | + | 88.6 | 97.46 | | |
| 273 | GCTC | | | | | | | |
| gRNA | CCTCTGTAACCCCTAA | 1028 | 1857 | + | 74.8 | 72.5 | | |
| 274 | GGTC | | | | | | | |
| gRNA | CTGCGCGGGAACCAGG | 1029 | 2011 | + | 96.1 | 91.67 | | |
| 275 | AGCC | | | | | | | |
| gRNA | GCTGGAAACGAGGTGT | 1030 | 1973 | − | 91.2 | 86.23 | | |
| 276 | TTCC | | | | | | | |
| gRNA | GGAACCAGGAGCCGGG | 1031 | 2018 | + | 77.2 | 85.92 | | |
| 277 | AACC | | | | | | | |
| gRNA | TTGCGGGGATGCTGGA | 1032 | 1983 | − | 100.2 | 79.17 | | |
| 278 | AACG | | | | | | | |
| gRNA | AGCTCCGGGTTCCCGG | 1033 | 2022 | − | 88.2 | 84.38 | | |
| 279 | CTCC | | | | | | | |

### Example 5: ZF Screening in Primary T Cells

This Example describes a study in which the ZFP domains targeting various genomic regions of the CIITA gene are subject to screening in human primary T cells.

T cells are isolated from human leukapheresis product and stored cryogenically. Prior to nucleofection, T cells are thawed, and stimulated with CD3/CD28 beads for approximately 48 hours in complete T cell medium at 37° C. with 5% CO₂. Beads are then magnetically removed from the culture and T cells are cultured in fresh complete T cell medium. T cells are nucleofected with ZF-off mRNA using the Lonza Amaxa 4D Nucleofector®. After nucleofection, T cells are resuspended in complete T cell medium and maintained by replacement of media and splitting of cells as necessary twice weekly. Cells are restimulated with soluble CD3/CD28 T Cell Activator on day 13 post-nucleofection. Cell surface HLA-DR protein expression on live T cells is assessed by flow cytometry at days 6, 13, and 20 post-nucleofection. No mRNA, non-CIITA targeting ZF-off mRNA, WT Cas9 mRNA plus exon-targeting gRNA, stain only, isotype, and no-stain controls are also run on each screening plate.

On days 6, 13, and 20 post-nucleofection, an aliquot of T cells is assessed by flow cytometric staining while a remaining split of cells continue to be maintained in culture. The cells are stained with PE-conjugated anti-human HLA-DR antibody and Fixable Viability Dye and are acquired on a flow cytometer, collecting up to 20,000 live-cell events per well. Screening conditions are compared to negative (CRISPR-off mRNA with no sgRNA) control expression levels to assess % silencing.

### Example 6: Full Specificity Screen of Constructs in Primary Human T Cells

The specificity of CRISPR-off and ZF-off constructs for silencing CIITA is tested in primary human T cells. The readouts to assess specificity are RNAseq, methylation array, and whole genome bisulfite sequencing assays. Genome-wide expression and methylation changes after epigenetic editing compared to negative controls are profiled.

### Example 7: CpG Methylation Patterns

The CpG methylation patterns in primary human T cells treated with CRISPR-off or ZF-off are investigated. Hybrid capture assay is performed on bisulfite treated DNA to investigate methylation patterns at CpG sites that are induced by CRISPR-off or ZF-off at the 1 kb region around the CIITA TSS.

### Example 8: Screen Follow-Up and Hit Validation

Top hits from gRNA and ZF-off screens are re-confirmed by repeating screening experimental conditions as well as adjusting doses of CRISPR-off mRNA+gRNA or ZF-off mRNA as appropriate upward and downward by several half logs to establish dose-response profiles. gRNAs and ZF-off mRNAs demonstrating the best potency and long-term durability profiles are selected for downstream candidate development.

### Example 9: Allogeneic Functional Assays in Primary T Cells

The response of allogeneic healthy donor CD4⁺ T cells to mock-modified or CIITA-silenced T cells is assessed via a mixed lymphocyte co-culture assay and/or a cytotoxicity assay.

Allogeneic healthy donor CD4⁺ T cell proliferation and/or activation, as measured by flow cytometry for cell dye dilution and cell surface expression of activation markers, respectively, are assessed after co-culture with T cells that are mock-modified or CIITA-silenced. A reduction of the response to CIITA-silenced cells, demonstrating less allogeneic healthy donor CD4⁺ T cell proliferation and activation, is expected relative to the response to mock-modified cells.

### Example 10: Additional Primary Screening

| | | Start | End | | SEQ ID |
|---|---|---|---|---|---|
| RNA ID | Sequence | Position | Position | Strand | NO: |
| RNA259 | CCGCTTGGGGCCACCTTGCA | 10877101 | 10877121 | + | 670 |
| RNA257 | ACCGCTTGGGGCCACCTTGC | 10877100 | 10877120 | + | 671 |
| RNA279 | GGCATCCGAAGGCATCCTTG | 10877241 | 10877261 | + | 672 |
| RNA273 | TAGTGATGAGGCTAGTGATG | 10877200 | 10877220 | + | 673 |
| RNA432 | AGTCTCCTCTGTAACCCCTA | 10879054 | 10879074 | + | 674 |
| RNA258 | CCCTGCAAGGTGGCCCCAAG | 10877104 | 10877124 | − | 675 |
| RNA289 | AGGAGGGGCTGCCAGACTCC | 10877278 | 10877298 | + | 676 |
| RNA235 | TCTGAACGTCAGACTGTTGA | 10876937 | 10876957 | + | 677 |
| RNA338 | GACCTGAAGTCTCCCTCCGG | 10877557 | 10877577 | − | 678 |
| RNA236 | TTGCACAGAAAGTCTGTTGG | 10876965 | 10876985 | − | 679 |
| RNA240 | ACCAAATTCAGTCCACAGTA | 10876997 | 10877017 | + | 680 |
| RNA266 | TGCATGTTGGCTTAGCTTGG | 10877157 | 10877177 | + | 681 |
| RNA241 | TCCTTACTGTGGACTGAATT | 10877001 | 10877021 | − | 682 |
| RNA272 | CTGGTGACTGGTTAGTGATG | 10877188 | 10877208 | + | 683 |
| RNA282 | GGCATCCTTGGGGAAGCTGA | 10877251 | 10877271 | + | 684 |
| RNA301 | AGGTAGGACCCAGCAGGGCG | 10877348 | 10877368 | − | 685 |
| RNA252 | AGGGAGTGTGGTAAAATTAG | 10877056 | 10877076 | + | 686 |
| RNA339 | GCTGACCTGAAGTCTCCCTC | 10877560 | 10877580 | − | 687 |
| RNA237 | GTTGCACAGAAAGTCTGTTG | 10876966 | 10876986 | − | 688 |
| RNA251 | GGGGAGGGCTTAAGGGAGTG | 10877044 | 10877064 | + | 689 |
| RNA253 | GGGAGTGTGGTAAAATTAGA | 10877057 | 10877077 | + | 690 |
| RNA278 | GGGCATCCGAAGGCATCCTT | 10877240 | 10877260 | + | 691 |
| RNA265 | CTTTGCATGTTGGCTTAGCT | 10877154 | 10877174 | + | 692 |
| RNA263 | AACATGCAAAGAAACAAGTG | 10877145 | 10877165 | − | 693 |
| RNA250 | GAGGTGTGGGGAGGGCTTAA | 10877037 | 10877057 | + | 694 |
| RNA213 | ATCTAAGAAGTCCCCAGCAG | 10876801 | 10876821 | + | 695 |
| RNA232 | ATTGGCAGCTGGCACCAGTG | 10876895 | 10876915 | + | 696 |
| RNA234 | TTCAGAAATGATGATCACAA | 10876923 | 10876943 | − | 697 |
| RNA431 | AGGGGTTACAGAGGAGACTT | 10879053 | 10879073 | − | 698 |
| RNA243 | GGAAGTGAAATTAATTTCAG | 10877018 | 10877038 | + | 699 |
| RNA316 | AGAGAAACCATTCTGAATTG | 10877450 | 10877470 | + | 1315 |
| RNA329 | AGTCTGTGTCCTGCTGGGGC | 10877496 | 10877516 | + | 1316 |
| RNA210 | GGGCAATTTGGTGTGGGAGT | 10876759 | 10876779 | + | 1317 |
| RNA434 | CCCGACCTTAGGGGTTACAG | 10879062 | 10879082 | − | 1318 |
| RNA222 | TCTTGAGGCATCTGGGCGGA | 10876835 | 10876855 | + | 1319 |
| RNA226 | CTGCCACCTTCTGCAGGATG | 10876873 | 10876893 | − | 1320 |
| RNA221 | GTCTTGAGGCATCTGGGCGG | 10876834 | 10876854 | + | 1321 |
| RNA308 | AGCCCCAAGGTAAAAAGGCC | 10877373 | 10877393 | + | 1322 |
| RNA220 | CTTGTCTTGAGGCATCTGGG | 10876831 | 10876851 | + | 1323 |
| RNA227 | TCTGCCACCTTCTGCAGGAT | 10876874 | 10876894 | − | 1324 |
| RNA238 | AGTTGCACAGAAAGTCTGTT | 10876967 | 10876987 | − | 1325 |
| RNA435 | CCTCTGTAACCCCTAAGGTC | 10879059 | 10879079 | + | 1326 |
| RNA274 | GAGGCTGTGTGCTTCTGAG | 10877219 | 10877238 | + | 1327 |
| RNA233 | TGATCACAATGGAACCGCAC | 10876912 | 10876932 | − | 1328 |
| RNA228 | ATCTGCCACCTTCTGCAGGA | 10876875 | 10876895 | − | 1329 |
| RNA212 | TGTGGGAGTAGGCATGGTAG | 10876770 | 10876790 | + | 1330 |
| RNA430 | GGTCACCTACCGCTGTTCCC | 10879032 | 10879052 | − | 1331 |
| RNA336 | TGAAGTCTCCCTCCGGTGGA | 10877553 | 10877573 | − | 1332 |
| RNA176 | TCCTCCTGGTGGTATCAAGG | 10876537 | 10876557 | − | 1333 |
| RNA230 | CTGCAGAAGGTGGCAGATAT | 10876877 | 10876897 | + | 1334 |
| RNA217 | GAGGCTCTCAGCTTGTCTTG | 10876820 | 10876840 | + | 1335 |
| RNA215 | ACAAGCTGAGAGCCTCTGCT | 10876816 | 10876836 | − | 1336 |
| RNA177 | CCATCCTCCTGGTGGTATCA | 10876540 | 10876560 | − | 1337 |
| RNA239 | AAGTTGCACAGAAAGTCTGT | 10876968 | 10876988 | − | 1338 |
| RNA219 | CAGCTTGTCTTGAGGCATCT | 10876828 | 10876848 | + | 1339 |
| RNA249 | AGAGGTGTGGGGAGGGCTTA | 10877036 | 10877056 | + | 1340 |
| RNA180 | GTCTCTGCCCATCCTCCTGG | 10876548 | 10876568 | − | 1341 |
| RNA410 | TGCCATGCTGCCTTCAGAGC | 10877950 | 10877970 | − | 1342 |
| RNA291 | AGACTCCGGGAGCTGCTGCC | 10877291 | 10877311 | + | 1343 |
| RNA216 | GACAAGCTGAGAGCCTCTGC | 10876817 | 10876837 | − | 1344 |
| RNA223 | TGGGCGGAGGGCTATGATAC | 10876847 | 10876867 | + | 1345 |
| RNA433 | TCCTCTGTAACCCCTAAGGT | 10879058 | 10879078 | + | 1346 |
| RNA175 | TCCTCCTTGATACCACCAGG | 10876533 | 10876553 | + | 1347 |
| RNA411 | CGGGGAGGTAGGATGACCAG | 10878933 | 10878953 | + | 1348 |
| RNA262 | ACATGCAAAGAAACAAGTGA | 10877144 | 10877164 | − | 1349 |
| RNA406 | ACAGTGCAGACACCTGGCAC | 10877928 | 10877948 | + | 1350 |
| RNA356 | AGGTCTTCTCGCCTCCCTTT | 10877690 | 10877710 | + | 1351 |
| RNA447 | CAAGCCAAGCTCCGGGTTCC | 10879234 | 10879254 | − | 1352 |
| RNA242 | TTAATTTCACTTCCTTACTG | 10877012 | 10877032 | − | 1353 |
| RNA225 | TGGCCCCATCCTGCAGAAGG | 10876867 | 10876887 | + | 1354 |
| RNA357 | CTCAGTATTTACCCAAAGGG | 10877704 | 10877724 | − | 1355 |
| RNA444 | GGAACCAGGAGCCGGGAACC | 10879220 | 10879240 | + | 1356 |
| RNA185 | AAATAATACTGACTCCCATA | 10876592 | 10876612 | − | 1357 |
| RNA401 | TAAAAAGGGTCAGGGGACGG | 10877886 | 10877906 | + | 1358 |
| RNA281 | AGGCATCCTTGGGGAAGCTG | 10877250 | 10877270 | + | 1359 |
| RNA409 | CACCGGCTCTGAAGGCAGCA | 10877945 | 10877965 | + | 1360 |
| RNA214 | CAAGCTGAGAGCCTCTGCTG | 10876815 | 10876835 | − | 1361 |
| RNA208 | AAATTCAGGGCAATTTGGTG | 10876752 | 10876772 | + | 1362 |
| RNA261 | TCAAAAAAACTCTCCCTGCA | 10877117 | 10877137 | − | 1363 |
| RNA342 | TGGTTCATACTCCAGACACC | 10877582 | 10877602 | − | 1364 |
| RNA179 | CTTGATACCACCAGGAGGAT | 10876538 | 10876558 | + | 1365 |
| RNA448 | GGCACAGCAAGCCAAGCTCC | 10879241 | 10879261 | − | 1366 |
| RNA173 | TCAAGGAGGAAGTCTCAGCT | 10876523 | 10876543 | − | 1367 |
| RNA255 | AAACAGAAATCTGACCGCTT | 10877087 | 10877107 | + | 1368 |
| RNA419 | CGGCCCCAGAGCTGGCGGGA | 10878978 | 10878998 | + | 1369 |
| RNA211 | ATTTGGTGTGGGAGTAGGCA | 10876764 | 10876784 | + | 1370 |
| RNA231 | AGGTGGCAGATATTGGCAGC | 10876884 | 10876904 | + | 1371 |
| RNA449 | GGGCACAGCAAGCCAAGCTC | 10879242 | 10879262 | − | 1372 |
| RNA297 | AGCCAGGCAACGCATTGTGT | 10877325 | 10877345 | − | 1373 |
| RNA184 | GACACTAACTTTCTCCCTAT | 10876575 | 10876595 | + | 1374 |
| RNA305 | ACCTTGGGGCTCTGACAGGT | 10877364 | 10877384 | − | 1375 |
| RNA247 | TTAATTTCAGAGGTGTGGGG | 10877028 | 10877048 | + | 1376 |
| RNA218 | TCAGCTTGTCTTGAGGCATC | 10876827 | 10876847 | + | 1377 |
| RNA174 | ACTTCCTCCTTGATACCACC | 10876530 | 10876550 | + | 1378 |
| RNA337 | TTCCACCGGAGGGAGACTTC | 10877552 | 10877572 | + | 1379 |
| RNA182 | CCAGTCTCTGCCCATCCTCC | 10876551 | 10876571 | − | 1380 |
| RNA192 | CCCAAACCATCTGACTACAA | 10876633 | 10876653 | + | 1381 |
| RNA203 | ATTTCTCTGGAGATCATAAG | 10876736 | 10876756 | − | 1382 |
| RNA290 | AGGCAGCAGCTCCCGGAGTC | 10877292 | 10877312 | − | 1383 |
| RNA368 | TCTGGGTATGGAGTGGAGTC | 10877754 | 10877774 | − | 1384 |
| RNA264 | TCACTTGTTTCTTTGCATGT | 10877144 | 10877164 | + | 1385 |
| RNA442 | TCTGCGCGGGAACCAGGAGC | 10879212 | 10879232 | + | 1386 |
| RNA254 | GAAACAGAAATCTGACCGCT | 10877086 | 10877106 | + | 1387 |
| RNA205 | ATGATCTCCAGAGAAATTCA | 10876739 | 10876759 | + | 1388 |
| RNA445 | AGCTCCGGGTTCCCGGCTCC | 10879227 | 10879247 | − | 1389 |
| RNA340 | AGGGAGACTTCAGGTCAGCC | 10877561 | 10877581 | + | 1390 |
| RNA178 | CCTTGATACCACCAGGAGGA | 10876537 | 10876557 | + | 1391 |
| RNA330 | GTCCTGCTGGGGCAGGCCAT | 10877503 | 10877523 | + | 1392 |
| RNA286 | GGAAGCTGAGGGCACGAGGA | 10877262 | 10877282 | + | 1393 |
| RNA181 | CCAGGAGGATGGGCAGAGAC | 10876548 | 10876568 | + | 1394 |
| RNA196 | GACTACAAGGGTACCATATT | 10876645 | 10876665 | + | 1395 |
| RNA428 | GGAGCCCGGGGAACAGCGGT | 10879024 | 10879044 | + | 1396 |
| RNA300 | ACCCAGCAGGGCGTGGAGCC | 10877341 | 10877361 | − | 1397 |
| RNA307 | GAGCCCCAAGGTAAAAAGGC | 10877372 | 10877392 | + | 1398 |
| RNA407 | ACACCTGGCACCGGCTCTGA | 10877937 | 10877957 | + | 1399 |
| RNA306 | GTCAGAGCCCCAAGGTAAAA | 10877368 | 10877388 | + | 1400 |
| RNA314 | ACAGAGAAACCATTCTGAAT | 10877448 | 10877468 | + | 1401 |
| RNA420 | TCTCCCTCCCGCCAGCTCTG | 10878984 | 10879004 | − | 1402 |
| RNA293 | CCCAGCCAGGCAGCAGCTCC | 10877299 | 10877319 | − | 1403 |
| RNA436 | GCTGGAAACGAGGTGTTTCC | 10879178 | 10879198 | − | 1404 |
| RNA437 | TTGCGGGGATGCTGGAAACG | 10879188 | 10879208 | − | 1405 |
| RNA202 | TTGGGATGAAAATGACAGGT | 10876710 | 10876730 | + | 1406 |
| RNA425 | AGGCCACCAGCAGCGCGCGC | 10879003 | 10879023 | + | 1407 |
| RNA186 | TAAATAATACTGACTCCCAT | 10876593 | 10876613 | − | 1408 |
| RNA365 | GAGGGGTGCCTCTGCAGGAC | 10877721 | 10877741 | + | 1409 |
| RNA195 | ATGGTACCCTTGTAGTCAGA | 10876642 | 10876662 | − | 1410 |
| RNA183 | AGACACTAACTTTCTCCCTA | 10876574 | 10876594 | + | 1411 |
| RNA405 | AAGAGCACAGTGCAGACACC | 10877922 | 10877942 | + | 1412 |
| RNA224 | TACTGGCCCCATCCTGCAGA | 10876864 | 10876884 | + | 1413 |
| RNA417 | TCCCGCCAGCTCTGGGGCCG | 10878978 | 10878998 | − | 1414 |
| RNA421 | CTCTCCCTCCCGCCAGCTCT | 10878985 | 10879005 | − | 1415 |
| RNA450 | GCTTGCTGTGCCCAGAGCTC | 10879249 | 10879269 | + | 1416 |
| RNA200 | TGTCTTGGGATGAAAATGAC | 10876706 | 10876726 | + | 1417 |
| RNA418 | GCGGCCCCAGAGCTGGCGGG | 10878977 | 10878997 | + | 1418 |
| RNA344 | AACCTTTCGGTGCTGATACA | 10877602 | 10877622 | − | 1419 |
| RNA343 | AACCATGTATCAGCACCGAA | 10877597 | 10877617 | + | 1420 |
| RNA209 | AATTCAGGGCAATTTGGTGT | 10876753 | 10876773 | + | 1421 |
| RNA191 | CCTTGTAGTCAGATGGTTTG | 10876635 | 10876655 | − | 1422 |
| RNA441 | AACGACTCTGCGCGGGAACC | 10879206 | 10879226 | + | 1423 |
| RNA294 | CCGGGAGCTGCTGCCTGGCT | 10877296 | 10877316 | + | 1424 |
| RNA187 | AGTATTATTTAGCATCACTT | 10876602 | 10876622 | + | 1425 |
| RNA427 | CGCGGGAGCCCGGGGAACAG | 10879020 | 10879040 | + | 1426 |
| RNA229 | CAATATCTGCCACCTTCTGC | 10876879 | 10876899 | − | 1427 |
| RNA194 | ACCCTTGTAGTCAGATGGTT | 10876637 | 10876657 | − | 1428 |
| RNA423 | CCTCTCCCTCCCGCCAGCTC | 10878986 | 10879006 | − | 1429 |
| RNA413 | GAGCTGCCACAGACTTGCCG | 10878958 | 10878978 | + | 1430 |
| RNA245 | GAAATTAATTTCAGAGGTGT | 10877024 | 10877044 | + | 1431 |
| RNA298 | TGCCTGGCTCCACGCCCTGC | 10877336 | 10877356 | + | 1432 |
| RNA347 | GTTCTAGAAGTCAGACTTTC | 10877619 | 10877639 | + | 1433 |
| RNA193 | CCCTTGTAGTCAGATGGTTT | 10876636 | 10876656 | − | 1434 |
| RNA438 | CAGAGTCGTTGCGGGGATGC | 10879196 | 10879216 | − | 1435 |
| RNA199 | TATTTGGGTTAACACTCTTT | 10876661 | 10876681 | + | 1436 |
| RNA390 | ACAACACCATTAGGAAACTG | 10877865 | 10877885 | − | 1437 |
| RNA415 | TGCCGCGGCCCCAGAGCTGG | 10878973 | 10878993 | + | 1438 |
| RNA443 | CTGCGCGGGAACCAGGAGCC | 10879213 | 10879233 | + | 1439 |
| RNA446 | GGAGCCGGGAACCCGGAGCT | 10879227 | 10879247 | + | 1440 |
| RNA397 | TGTTGTTTAAAAAGGGTCAG | 10877879 | 10877899 | + | 1441 |
| RNA439 | ATCCCCGCAACGACTCTGCG | 10879198 | 10879218 | + | 1442 |
| RNA367 | TATGGAGTGGAGTCTGGCAG | 10877748 | 10877768 | − | 1443 |
| RNA321 | CCCCCATCCCCAATTCAGAA | 10877460 | 10877480 | − | 1444 |
| RNA201 | CTTGGGATGAAAATGACAGG | 10876709 | 10876729 | + | 1445 |
| RNA172 | GAGTGATCATATATTAGAAG | 10876478 | 10876498 | + | 1446 |
| RNA351 | GCCTGGCTCGGCCCACAGCA | 10877670 | 10877690 | + | 1447 |
| RNA318 | AACCATTCTGAATTGGGGAT | 10877455 | 10877475 | + | 1448 |
| RNA398 | GTTTAAAAAGGGTCAGGGGA | 10877883 | 10877903 | + | 1449 |
| RNA322 | CTGAATTGGGGATGGGGGTG | 10877462 | 10877482 | + | 1450 |
| RNA260 | AAAAAACTCTCCCTGCAAGG | 10877114 | 10877134 | − | 1451 |
| RNA424 | GAGGCCACCAGCAGCGCGCG | 10879002 | 10879022 | + | 1452 |
| RNA396 | GTGTTGTTTAAAAAGGGTCA | 10877878 | 10877898 | + | 1453 |
| RNA384 | CAGCAGCTGGGGAGAGCTGG | 10877827 | 10877847 | + | 1454 |
| RNA366 | GCAGAGGTCCCGTCCTGCAG | 10877732 | 10877752 | − | 1455 |
| RNA198 | AAAGAGTGTTAACCCAAATA | 10876661 | 10876681 | − | 1456 |
| RNA414 | ACTTGCCGCGGCCCCAGAGC | 10878970 | 10878990 | + | 1457 |
| RNA345 | GTCTGACTTCTAGAACCTTT | 10877615 | 10877635 | − | 1458 |
| RNA197 | ACTACAAGGGTACCATATTT | 10876646 | 10876666 | + | 1459 |
| RNA188 | ATTATTTAGCATCACTTTGG | 10876605 | 10876625 | + | 1460 |
| RNA190 | CCCCAAACCATCTGACTACA | 10876632 | 10876652 | + | 1461 |
| RNA429 | GTCACCTACCGCTGTTCCCC | 10879031 | 10879051 | − | 1462 |
| RNA358 | CTCCCTTTGGGTAAATACTG | 10877702 | 10877722 | + | 1463 |
| RNA248 | TAATTTCAGAGGTGTGGGGA | 10877029 | 10877049 | + | 1464 |
| RNA440 | TCCCCGCAACGACTCTGCGC | 10879199 | 10879219 | + | 1465 |
| RNA374 | AGAAGCAGGGAAACCAAAAT | 10877773 | 10877793 | + | 1466 |
| RNA346 | GGTTCTAGAAGTCAGACTTT | 10877618 | 10877638 | + | 1467 |
| RNA412 | GTCTGTGGCAGCTCGTCCGC | 10878952 | 10878972 | − | 1468 |
| RNA207 | CAGAGAAATTCAGGGCAATT | 10876747 | 10876767 | + | 1469 |
| RNA244 | TGAAATTAATTTCAGAGGTG | 10877023 | 10877043 | + | 1470 |
| RNA189 | TTATTTAGCATCACTTTGGC | 10876606 | 10876626 | + | 1471 |
| RNA389 | CAACACCATTAGGAAACTGG | 10877864 | 10877884 | − | 1472 |
| RNA350 | CTCAGCATGCTGGCCTGGCT | 10877658 | 10877678 | + | 1473 |
| RNA256 | AACAGAAATCTGACCGCTTG | 10877088 | 10877108 | + | 1474 |
| RNA392 | AAACAACACCATTAGGAAAC | 10877867 | 10877887 | − | 1475 |
| RNA375 | CTCAAGGCTGACTCCAATTT | 10877789 | 10877809 | − | 1476 |
| RNA382 | CCAGCTCTCCCCAGCTGCTG | 10877827 | 10877847 | − | 1477 |
| RNA204 | TATGATCTCCAGAGAAATTC | 10876738 | 10876758 | + | 1478 |
| RNA206 | CAAATTGCCCTGAATTTCTC | 10876749 | 10876769 | − | 1479 |
| RNA404 | GGGACGGGGGAACAGATGGT | 10877899 | 10877919 | + | 1480 |
| RNA391 | AACAACACCATTAGGAAACT | 10877866 | 10877886 | − | 1481 |
| RNA383 | CCTCAGCAGCTGGGGAGAGC | 10877824 | 10877844 | + | 1482 |
| RNA359 | TCCCTTTGGGTAAATACTGA | 10877703 | 10877723 | + | 1483 |
| RNA246 | AAATTAATTTCAGAGGTGTG | 10877025 | 10877045 | + | 1484 |
| RNA313 | GAATGGTTTCTCTGTTTATC | 10877443 | 10877463 | − | 1485 |
| RNA422 | CCAGAGCTGGCGGGAGGGAG | 10878983 | 10879003 | + | 1486 |
| RNA360 | CCCCTCAGTATTTACCCAAA | 10877707 | 10877727 | − | 1487 |
| RNA348 | GTCACTAACTCTCAGCATGC | 10877648 | 10877668 | + | 1488 |
| RNA349 | TAACTCTCAGCATGCTGGCC | 10877653 | 10877673 | + | 1489 |
| RNA408 | CTGCCTTCAGAGCCGGTGCC | 10877943 | 10877963 | − | 1490 |
| RNA426 | GCTCCCGCGCGCGCTGCTGG | 10879009 | 10879029 | − | 1491 |
| RNA352 | ACCTTGCTGTGGGCCGAGCC | 10877674 | 10877694 | − | 1492 |
| RNA400 | TTAAAAAGGGTCAGGGGACG | 10877885 | 10877905 | + | 1493 |
| RNA399 | TTTAAAAAGGGTCAGGGGAC | 10877884 | 10877904 | + | 1494 |
| RNA376 | CAAAATTGGAGTCAGCCTTG | 10877787 | 10877807 | + | 1495 |
| RNA381 | CAGCTCTCCCCAGCTGCTGA | 10877826 | 10877846 | − | 1496 |
| RNA416 | GCCGCGGCCCCAGAGCTGGC | 10878974 | 10878994 | + | 1497 |
| RNA259 | CCGCTTGGGGCCACCTTGCA | 10877101 | 10877121 | + | 1498 |
| RNA268 | TAGCTTGGCGGGCTCCCAAC | 10877169 | 10877189 | + | 1499 |
| RNA269 | GCGGGCTCCCAACTGGTGAC | 10877176 | 10877196 | + | 1500 |
| RNA236 | TTGCACAGAAAGTCTGTTGG | 10876965 | 10876985 | − | 1501 |
| RNA273 | TAGTGATGAGGCTAGTGATG | 10877200 | 10877220 | + | 1502 |
| RNA275 | AGGCTGTGTGCTTCTGAGCT | 10877220 | 10877240 | + | 1503 |
| RNA283 | TCGTGCCCTCAGCTTCCCCA | 10877259 | 10877279 | − | 1504 |
| RNA251 | GGGGAGGGCTTAAGGGAGTG | 10877044 | 10877064 | + | 1505 |
| RNA270 | TCACTAACCAGTCACCAGTT | 10877186 | 10877206 | − | 1506 |
| RNA279 | GGCATCCGAAGGCATCCTTG | 10877241 | 10877261 | + | 1507 |
| RNA301 | AGGTAGGACCCAGCAGGGCG | 10877348 | 10877368 | − | 1508 |
| RNA220 | CTTGTCTTGAGGCATCTGGG | 10876831 | 10876851 | + | 1509 |
| RNA267 | GCATGTTGGCTTAGCTTGGC | 10877158 | 10877178 | + | 1510 |
| RNA241 | TCCTTACTGTGGACTGAATT | 10877001 | 10877021 | − | 1511 |
| RNA257 | ACCGCTTGGGGCCACCTTGC | 10877100 | 10877120 | + | 1512 |
| RNA235 | TCTGAACGTCAGACTGTTGA | 10876937 | 10876957 | + | 1513 |
| RNA250 | GAGGTGTGGGGAGGGCTTAA | 10877037 | 10877057 | + | 1514 |
| RNA285 | GGGAAGCTGAGGGCACGAGG | 10877261 | 10877281 | + | 1515 |
| RNA372 | TTTCCCTGCTTCTCTGGGTA | 10877766 | 10877786 | − | 1516 |
| RNA403 | GGGGACGGGGGAACAGATGG | 10877898 | 10877918 | + | 1517 |
| RNA380 | TGTTGAGCCCTCAGCAGCTG | 10877816 | 10877836 | + | 1518 |
| RNA388 | AACACCATTAGGAAACTGGG | 10877863 | 10877883 | − | 1519 |
| RNA335 | GTCTATTTCCTTCCACCGGA | 10877542 | 10877562 | + | 1520 |
| RNA386 | CCCTCCCCCCAGTTTCCTAA | 10877856 | 10877876 | + | 1521 |
| RNA363 | ATACTGAGGGGTGCCTCTGC | 10877716 | 10877736 | + | 1522 |
| RNA324 | TTGGGGATGGGGGTGAGGAT | 10877467 | 10877487 | + | 1523 |
| RNA332 | AACTCTTTCACATCTTCCAA | 10877522 | 10877542 | − | 1524 |
| RNA312 | GTTTCTCTGTTTATCTGGAA | 10877438 | 10877458 | − | 1525 |
| RNA320 | CCATTCTGAATTGGGGATGG | 10877457 | 10877477 | + | 1526 |
| RNA315 | CAGAGAAACCATTCTGAATT | 10877449 | 10877469 | + | 1527 |

### Example 11: CIITA Secondary Screening

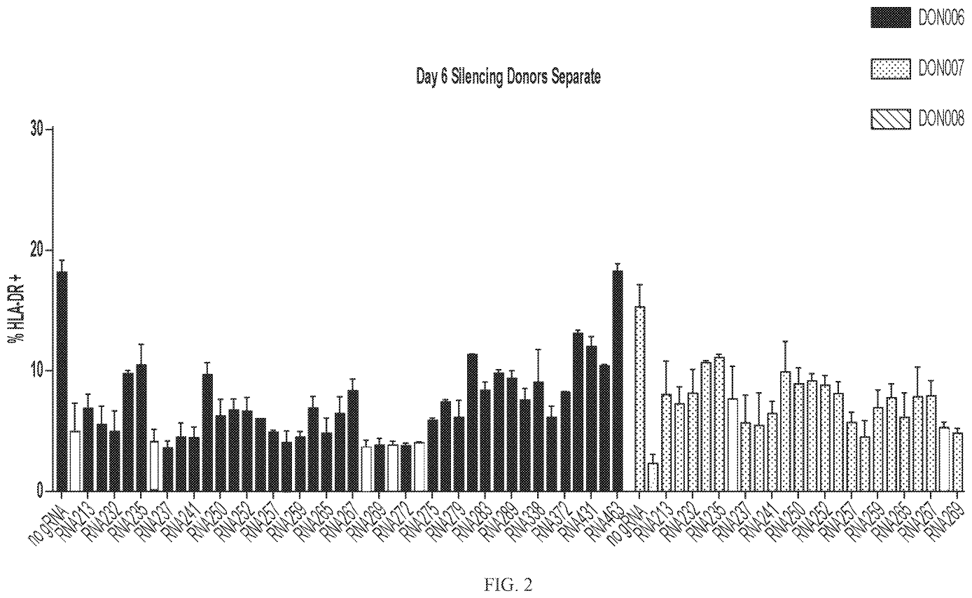

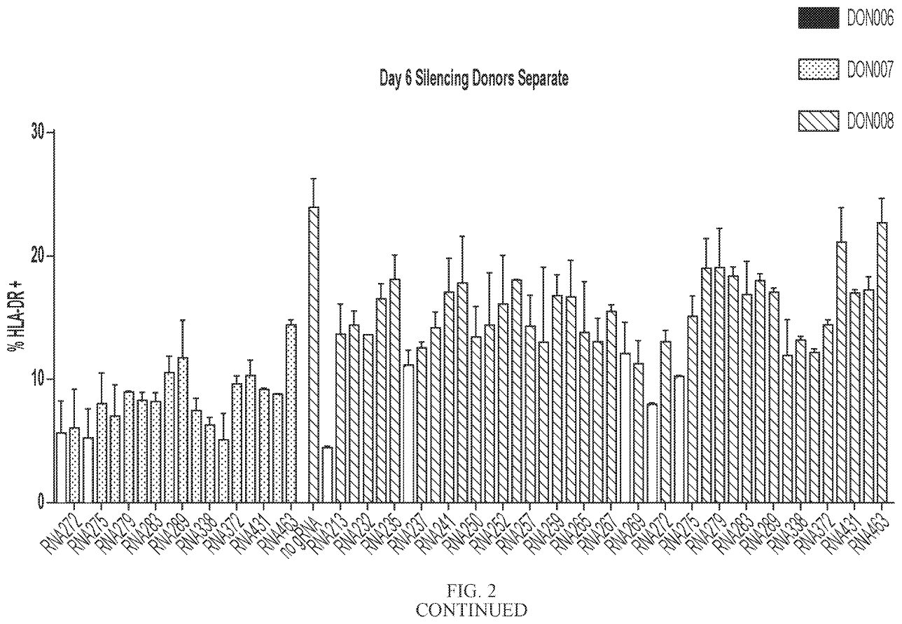

The top gRNAs from the previously-described primary screen were selected for secondary screening. Primary human T cells from donors DON006, DON007, and DON008 were nucleofected with 2.5 mg of gRNA and 2.5 mg of effector. Results are displayed by donor in FIG. 2.

### Example 12: CIITA Dual Guide Screening

This Example describes a study in which gRNAs were screened in pairs for their efficacy in targeting CIITA in primary human T cells.

T cells were isolated from human leukapheresis product (StemCell Technologies, Cat. No. 70500) using the EasySep™ Human T cell Isolation Kit (StemCell Technologies, Cat. No. 17951). Prior to nucleofection, T cells were thawed, washed, and stimulated using Dynabeads Human T-Activator CD3/CD28 for T Cell Expansion and Activation (Thermo Fisher, Cat. No. 11131D) at a 3:1 bead-to-cell number ratio for approximately 72 hours at 37° C. with 5% CO₂ in T cell medium (X-VIVO15 media (Lonza®, Cat. No. BEBP04-744Q) supplemented with 5% Human AB serum (Gemini Bio-Product, Cat. No. 100-512), 2 mM L-alanyl-L-glutamine, 100 U/ml IL-2, 10 ng/ml IL-7, and 10 ng/ml IL-15. Beads were then magnetically removed from the culture and T cells were cultured in fresh complete T cell medium for approximately 24 hours. T cells were then nucleofected with 5 μg CRISPR-off mRNA (TriLink) plus 2.5 μg sgRNA (IDT) at 2E5 cells/well using the P3 Primary Cell 96 -well Nucleofector Kit (Lonza®, Cat. No. V4SP-3960) and the Amaxa 4D Nucleofector® (Lonza) with pulse code EO115.

After nucleofection, T cells were resuspended in complete T cell medium and maintained by replacement of media and passages as necessary twice weekly. Cells were restimulated with ImmunoCult™ Human CD3/CD28 T Cell Activator (StemCell Technologies, Cat. No. 10991) on day 13 post-nucleofection.

Cell surface HLA-DR protein expression on live T cells was assessed by flow cytometry at days 6 and 20 post-nucleofection. No mRNA, CRISPR-off mRNA plus non-CIITA targeting sgRNA, CRISPR-off mRNA with no gRNA, WT Cas9 mRNA plus exon-targeting sgRNA, stain only (no mRNA or gRNA), isotype (no mRNA or gRNA), and no-stain (no mRNA or gRNA) controls were also run on each screening plate.

### HLA-DR Flow Cytometry

On days 6 and 20 post-nucleofection, an aliquot of T cells was assessed by flow cytometric staining while a remaining split of cells continued to be maintained in culture. The cells to be stained had media aspirated, were washed once with PBS containing 2% FBS, and were stained with PE-conjugated anti-human HLA-DR Antibody (BioLegend, Cat. No.327008) at a 1:300 dilution and Zombie Violet™ Fixable Viability Dye (BioLegend, Cat. No. 423113), previously prepared according to manufacturer's recommendations, at a 1:1000 dilution in PBS with 2% FBS at 4° C. for 20 minutes. The stained cells were washed and incubated in Fixation Buffer (BioLegend, Cat. No. 420801) for 20 minutes. The cells were then washed prior to acquisition on an Agilent Novocyte Penteon flow cytometer, which could collect up to 20,000 live-cell events per well. Screening conditions were compared to negative (CRISPR-Off mRNA with no sgRNA) control expression levels to assess % silencing.

### Results

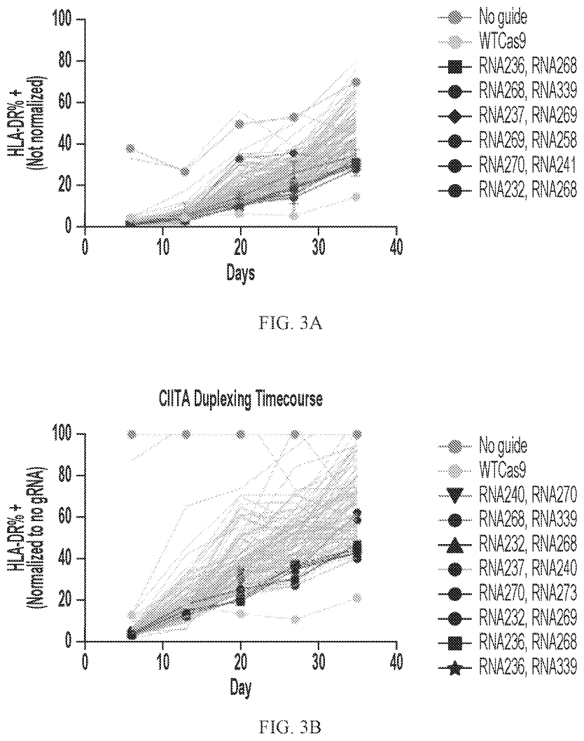

A timecourse of individual pairs is shown in FIG. 3A (not normalized) and FIG. 3B (normalized). Many pairs showed a similar silencing profile to WT Cas9 (shown).

### Example 13: Dose Response for Dual Guides

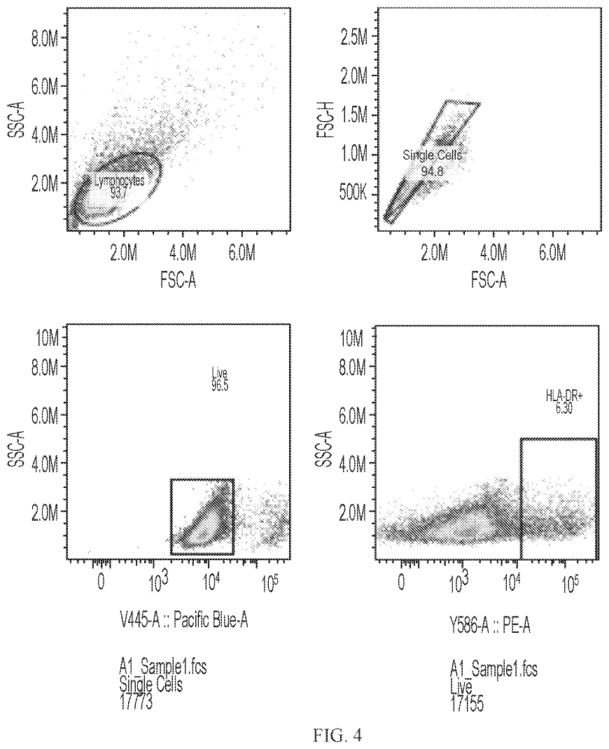

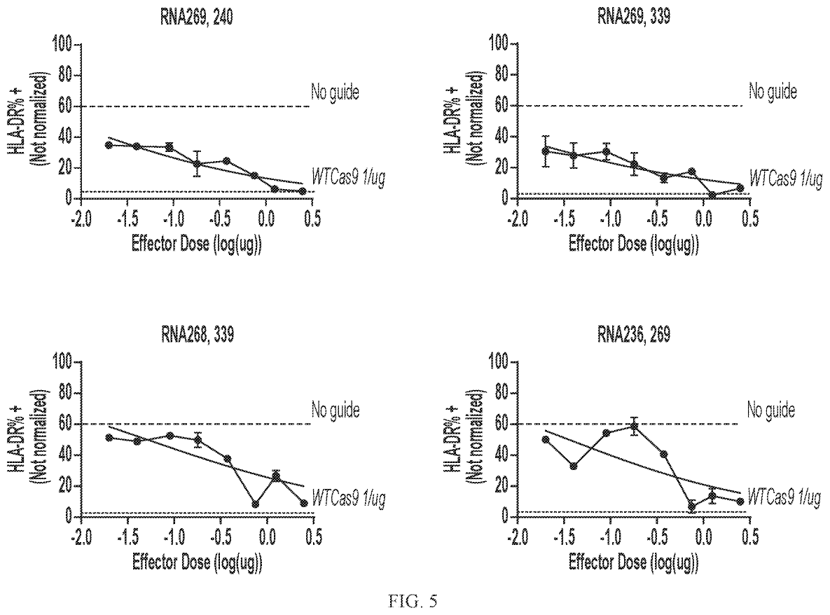

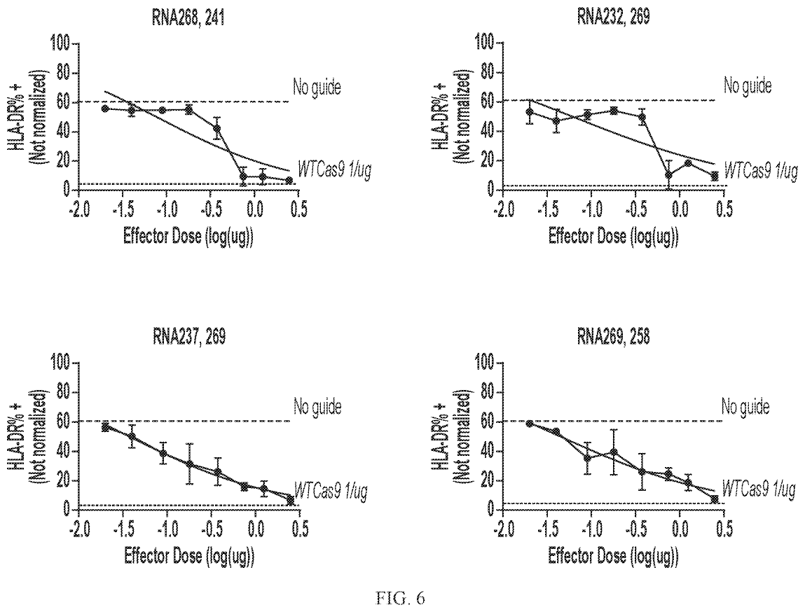

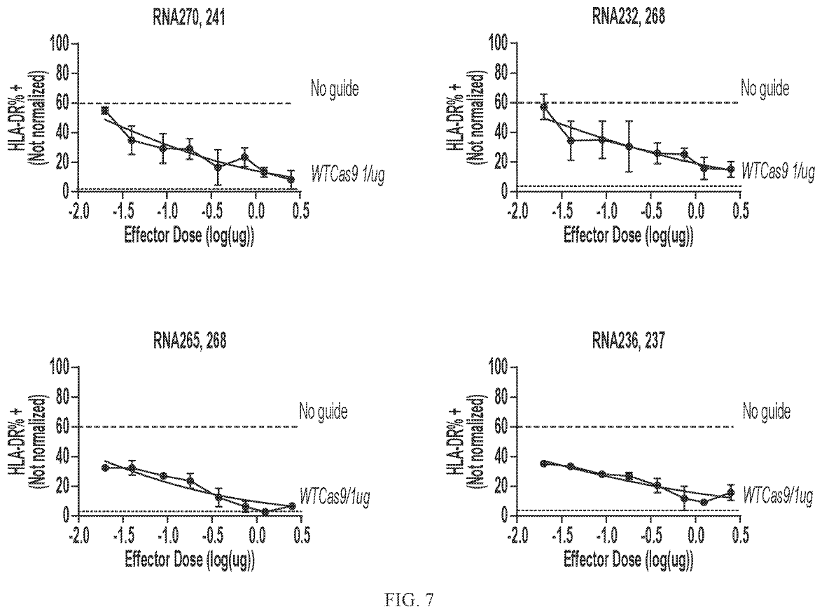

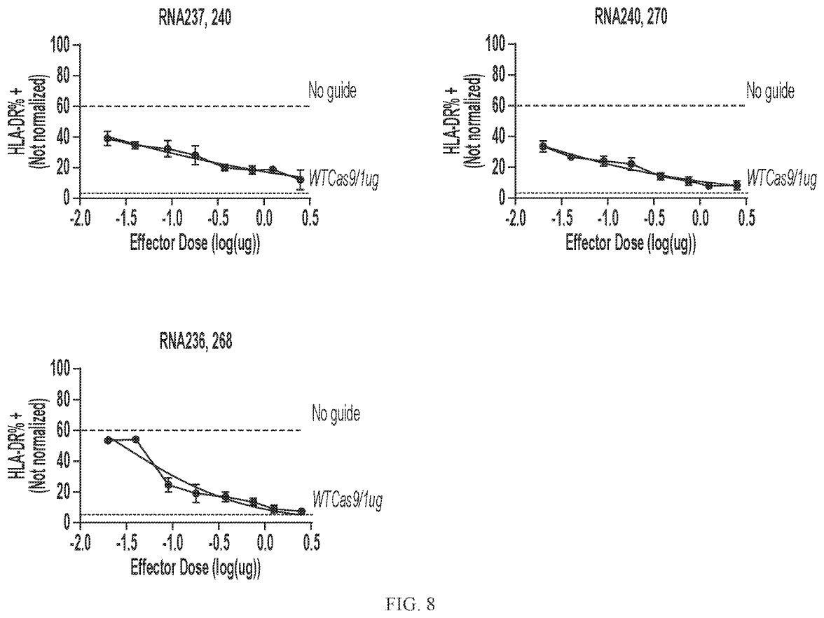

The dose response of fifteen guide pairs was assayed at two points. 2.5 micrograms of Fusion Protein 11a was used, as well as a starting dose of 2.5 micrograms each sgRNA. Response was observed on days 6. Gating strategy and results are shown in FIG. 4-FIG. 8.

### Example 14: ZF Screening in Primary T Cells

This Example describes a study in which the ZFP domains targeting various genomic regions of the CIITA gene were subject to screening in human primary T cells.

T cells were isolated from human leukapheresis product and stored cryogenically. Prior to nucleofection, T cells were thawed, and stimulated with CD3/CD28 beads for approximately 48 hours in complete T cell medium at 37° C. with 5% CO₂. Beads were then magnetically removed from the culture and T cells were cultured in fresh complete T cell medium. T cells were nucleofected with one of 233 ZF-off mRNAs (2.5 micrograms per well of 200k cells) using Viaflo. After nucleofection, T cells were resuspended in complete T cell medium and maintained by replacement of media and splitting of cells as necessary twice weekly. Cells were restimulated with soluble CD3/CD28 T Cell Activator on day 13 post-nucleofection. Cell surface HLA-DR protein expression on live T cells was assessed by flow cytometry at days 6, 13, and 20 post-nucleofection. No mRNA, non-CIITA targeting ZF-off mRNA, WT Cas9 mRNA plus exon-targeting gRNA, stain only, isotype, and no-stain controls were also run on each screening plate.

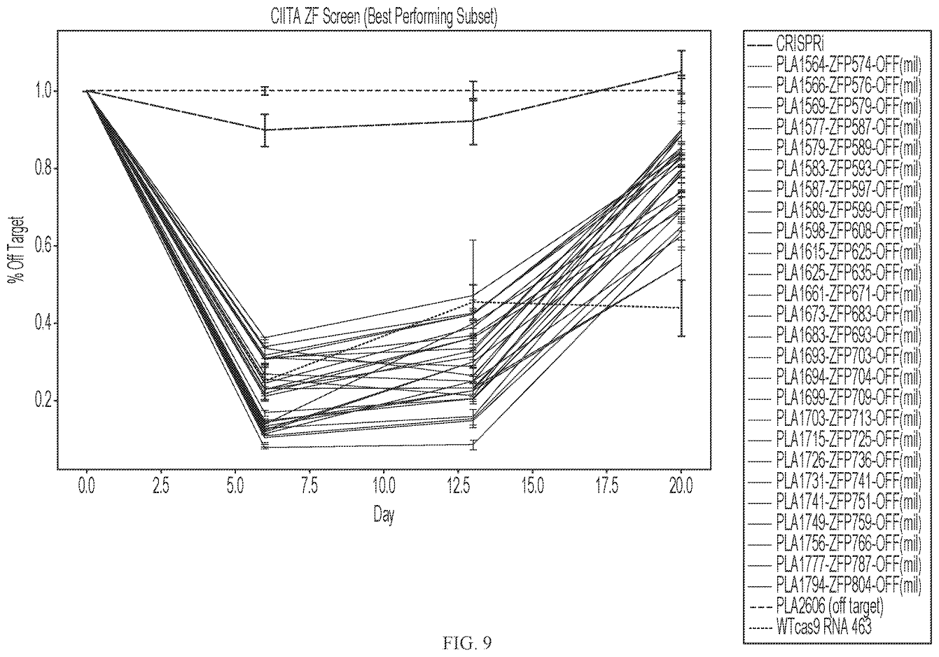

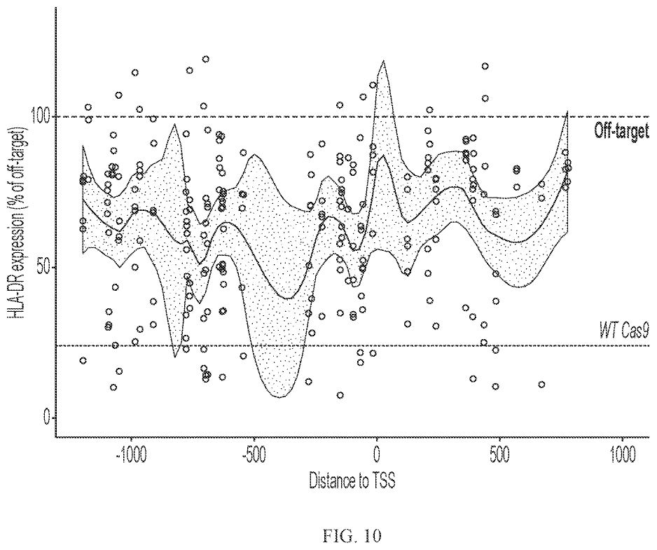

On days 6, 13, and 20 post-nucleofection, an aliquot of T cells was assessed by flow cytometric staining while a remaining split of cells continue to be maintained in culture. The cells were stained with PE-conjugated anti-human HLA-DR antibody and Fixable Viability Dye and were acquired on a flow cytometer, collecting up to 20,000 live-cell events per well. Screening conditions were compared to negative (CRISPR-off mRNA with no sgRNA) control expression levels to assess % silencing. A timecourse of the screen is shown in FIG. 9, and the distances of the ZFPs tested from the CIITA TSS is shown in FIG. 10.

10 ZFs with less than 5% HLA-DR expression were identified in this screen, and many more in the 5-10% range. Note that the ZFPs are often referred to as “ZF ###” in this application.

| | | | | | | | % Off |
|---|---|---|---|---|---|---|---|
| | SEQ | | Start | | | | Target |
| TAR | ID NO: | Sequence | Position | End Position | Name | % CD3+ | Control |
| TAR1625 | 1528 | GGAGGACCCAGCAGGAATC | 10878400 | 10878420 | pos: 3699- | 27.50 | 0.79 |
| | | | | | 3720 | | |
| TAR1648 | 1529 | GAGAGCCTCTGCTGGGGAC | 10876809 | 10876829 | pos: 2110- | 32.60 | 0.93 |
| | | | | | 2128 | | |
| TAR1649 | 1530 | ACAGAAAGTCTGTTGGGGG | 10876961 | 10876981 | pos: 2262- | 26.66 | 0.76 |
| | | | | | 2280 | | |
| TAR1651 | 1531 | GTGGACTGAATTTGGTGAA | 10876993 | 10877013 | pos: 2294- | 29.70 | 0.85 |
| | | | | | 2312 | | |
| TAR1653 | 1532 | TAGGACCCAGCAGGGCGTG | 10877345 | 10877365 | pos: 2646- | 26.65 | 0.76 |
| | | | | | 2664 | | |
| TAR1654 | 1533 | GACAGGTAGGACCCAGCAG | 10877351 | 10877371 | pos: 2652- | 25.27 | 0.72 |
| | | | | | 2670 | | |
| TAR1656 | 1534 | GGGGAGGGCAGCATCCGCC | 10877844 | 10877864 | pos: 3145- | 17.63 | 0.50 |
| | | | | | 3163 | | |
| TAR1646 | 1535 | GTGGTATCAAGGAGGAAGT | 10876529 | 10876549 | pos: 1830- | 25.60 | 0.73 |
| | | | | | 1848 | | |
| TAR1559 | 1536 | GGGTGTTCAGAAACAGAA | 10877076 | 10877095 | pos: 2375- | 28.05 | 0.80 |
| | | | | | 2395 | | |
| TAR1580 | 1537 | GGAGTCTGAGAAAAGGGA | 10878188 | 10878207 | pos: 3489- | 17.55 | 0.50 |
| | | | | | 3506 | | |
| TAR1607 | 1538 | AGGGCACGAGGAGGGGCTG | 10877269 | 10877289 | pos: 2568- | 6.46 | 0.18 |
| | | | | | 2589 | | |
| N/A | | | | | | 35.65 | 1.02 |
| TAR1625 | 1528 | GGAGGACCCAGCAGGAATC | 10878400 | 10878420 | pos: 3699- | 28.15 | 0.80 |
| | | | | | 3720 | | |
| TAR1648 | 1529 | GAGAGCCTCTGCTGGGGAC | 10876809 | 10876829 | pos: 2110- | 27.75 | 0.79 |
| | | | | | 2128 | | |
| TAR1649 | 1530 | ACAGAAAGTCTGTTGGGGG | 10876961 | 10876981 | pos: 2262- | 10.70 | 0.31 |
| | | | | | 2280 | | |
| TAR1651 | 1531 | GTGGACTGAATTTGGTGAA | 10876993 | 10877013 | pos: 2294- | 30.35 | 0.87 |
| | | | | | 2312 | | |
| TAR1653 | 1532 | TAGGACCCAGCAGGGCGTG | 10877345 | 10877365 | pos: 2646- | 22.26 | 0.64 |
| | | | | | 2664 | | |
| TAR1654 | 1533 | GACAGGTAGGACCCAGCAG | 10877351 | 10877371 | pos: 2652- | 17.20 | 0.49 |
| | | | | | 2670 | | |
| TAR1656 | 1534 | GGGGAGGGCAGCATCCGCC | 10877844 | 10877864 | pos: 3145- | 30.25 | 0.86 |
| | | | | | 3163 | | |
| TAR1647 | 1539 | GTAGTCAGATGGTTTGGGG | 10876631 | 10876651 | pos: 1932- | 28.81 | 0.82 |
| | | | | | 1950 | | |
| TAR1559 | 1536 | GGGTGTTCAGAAACAGAA | 10877076 | 10877095 | pos: 2375- | 20.85 | 0.60 |
| | | | | | 2395 | | |
| TAR1582 | 1540 | GCATGAGACTTTGAGGTG | 10878379 | 10878398 | pos: 3680- | 34.75 | 0.99 |
| | | | | | 3697 | | |
| TAR1609 | 1541 | TGGGGATGGGGGTGAGGAT | 10877467 | 10877487 | pos: 2766- | 28.30 | 0.81 |
| | | | | | 2787 | | |
| N/A | | | | | | 4.13 | 0.12 |
| TAR1625 | 1528 | GGAGGACCCAGCAGGAATC | 10878400 | 10878420 | pos: 3699- | 27.60 | 0.79 |
| | | | | | 3720 | | |
| TAR1648 | 1529 | GAGAGCCTCTGCTGGGGAC | 10876809 | 10876829 | pos: 2110- | 4.51 | 0.13 |
| | | | | | 2128 | | |
| TAR1649 | 1530 | ACAGAAAGTCTGTTGGGGG | 10876961 | 10876981 | pos: 2262- | 20.80 | 0.59 |
| | | | | | 2280 | | |
| TAR1651 | 1531 | GTGGACTGAATTTGGTGAA | 10876993 | 10877013 | pos: 2294- | 16.85 | 0.48 |
| | | | | | 2312 | | |
| TAR1653 | 1532 | TAGGACCCAGCAGGGCGTG | 10877345 | 10877365 | pos: 2646- | 20.98 | 0.60 |
| | | | | | 2664 | | |
| TAR1655 | 1542 | TGGGTATGGAGTGGAGTCT | 10877752 | 10877772 | pos: 3053- | 26.10 | 0.75 |
| | | | | | 3071 | | |
| TAR1656 | 1534 | GGGGAGGGCAGCATCCGCC | 10877844 | 10877864 | pos: 3145- | 32.40 | 0.93 |
| | | | | | 3163 | | |
| TAR1650 | 1543 | GAATTTGGTGAAGACAGAA | 10876986 | 10877006 | pos: 2287- | 35.85 | 1.02 |
| | | | | | 2305 | | |
| TAR1561 | 1544 | GGTCAGGGGACGGGGGAA | 10877892 | 10877911 | pos: 3191- | 33.55 | 0.96 |
| | | | | | 3211 | | |
| TAR1582 | 1545 | GCATGAGACTTTGAGGTG | 10878379 | 10878398 | pos: 3680- | 36.15 | 1.03 |
| | | | | | 3697 | | |
| TAR1610 | 1546 | TGGGGGTGAGGATGGGAAC | 10877473 | 10877493 | pos: 2772- | 30.71 | 0.88 |
| | | | | | 2793 | | |
| N/A | | | | | | 31.00 | 0.89 |
| TAR1646 | 1547 | GTGGTATCAAGGAGGAAGT | 10876529 | 10876549 | pos: 1830- | 3.88 | 0.11 |
| | | | | | 1848 | | |
| TAR1648 | 1548 | GAGAGCCTCTGCTGGGGAC | 10876809 | 10876829 | pos: 2110- | 25.31 | 0.72 |
| | | | | | 2128 | | |
| TAR1650 | 1549 | GAATTTGGTGAAGACAGAA | 10876986 | 10877006 | pos: 2287- | 13.65 | 0.39 |
| | | | | | 2305 | | |
| TAR1652 | 1550 | GCAACGCATTGTGTAGGAA | 10877319 | 10877339 | pos: 2620- | 24.33 | 0.69 |
| | | | | | 2638 | | |
| TAR1653 | 1551 | TAGGACCCAGCAGGGCGTG | 10877345 | 10877365 | pos: 2646- | 27.76 | 0.79 |
| | | | | | 2664 | | |
| TAR1655 | 1552 | TGGGTATGGAGTGGAGTCT | 10877752 | 10877772 | pos: 3053- | 24.42 | 0.70 |
| | | | | | 3071 | | |
| TAR186 | 1553 | GAGGCTGTGTGCTTCTGAG | 10877218 | 10877238 | 0 | 7.53 | 0.22 |
| TAR186 | 1554 | GAGGCTGTGTGCTTCTGAG | 10877218 | 10877238 | 0 | 38.80 | 1.11 |
| TAR1562 | 1555 | GGCTGGGAAGGGTGTGCC | 10877980 | 10877999 | pos: 3279- | 33.10 | 0.95 |
| | | | | | 3299 | | |
| TAR1602 | 1556 | GCAATTTGGTGTGGGAGTA | 10876760 | 10876780 | pos: 2059- | 40.95 | 1.17 |
| | | | | | 2080 | | |
| TAR1616 | 1557 | GAACAGATGGTGGGAAGAG | 10877907 | 10877927 | pos: 3206- | 5.76 | 0.16 |
| | | | | | 3227 | | |
| N/A | | | | | | 32.90 | 0.94 |
| TAR1646 | 1558 | GTGGTATCAAGGAGGAAGT | 10876529 | 10876549 | pos: 1830- | 27.19 | 0.78 |
| | | | | | 1848 | | |
| TAR1648 | 1559 | GAGAGCCTCTGCTGGGGAC | 10876809 | 10876829 | pos: 2110- | 11.81 | 0.34 |
| | | | | | 2128 | | |
| TAR1650 | 1560 | GAATTTGGTGAAGACAGAA | 10876986 | 10877006 | pos: 2287- | 31.91 | 0.91 |
| | | | | | 2305 | | |
| TAR1652 | 1561 | GCAACGCATTGTGTAGGAA | 10877319 | 10877339 | pos: 2620- | 15.96 | 0.46 |
| | | | | | 2638 | | |
| TAR1654 | 1562 | GACAGGTAGGACCCAGCAG | 10877351 | 10877371 | pos: 2652- | 30.51 | 0.87 |
| | | | | | 2670 | | |
| TAR1655 | 1563 | TGGGTATGGAGTGGAGTCT | 10877752 | 10877772 | pos: 3053- | 15.20 | 0.43 |
| | | | | | 3071 | | |
| TAR186 | 1564 | GAGGCTGTGTGCTTCTGAG | 10877218 | 10877238 | 0 | 30.65 | 0.88 |
| TAR1564 | 1565 | TAAGTTGCTACGGGAAAG | 10878276 | 10878295 | pos: 3575- | 31.15 | 0.89 |
| | | | | | 3595 | | |
| TAR1602 | 1566 | GCAATTTGGTGTGGGAGTA | 10876760 | 10876780 | pos: 2059- | 37.22 | 1.06 |
| | | | | | 2080 | | |
| TAR1616 | 1567 | GAACAGATGGTGGGAAGAG | 10877907 | 10877927 | pos: 3206- | 36.34 | 1.04 |
| | | | | | 3227 | | |
| N/A | | | | | | 0.00 | 0.00 |
| TAR1647 | 1568 | GTAGTCAGATGGTTTGGGG | 10876631 | 10876651 | pos: 1932- | 29.05 | 0.83 |
| | | | | | 1950 | | |
| TAR1649 | 1569 | ACAGAAAGTCTGTTGGGGG | 10876961 | 10876981 | pos: 2262- | 25.23 | 0.72 |
| | | | | | 2280 | | |
| TAR1651 | 1570 | GTGGACTGAATTTGGTGAA | 10876993 | 10877013 | pos: 2294- | 28.89 | 0.82 |
| | | | | | 2312 | | |
| TAR1652 | 1571 | GCAACGCATTGTGTAGGAA | 10877319 | 10877339 | pos: 2620 | 30.30 | 0.87 |
| | | | | | 2638 | | |
| TAR1654 | 1572 | GACAGGTAGGACCCAGCAG | 10877351 | 10877371 | pos: 2652- | 2.64 | 0.08 |
| | | | | | 2670 | | |
| TAR1656 | 1573 | GGGGAGGGCAGCATCCGCC | 10877844 | 10877864 | pos: 3145- | 29.15 | 0.83 |
| | | | | | 3163 | | |
| TAR186 | 1574 | GAGGCTGTGTGCTTCTGAG | 10877218 | 10877238 | 0 | 31.58 | 0.90 |
| TAR1577 | 1575 | CTGGAATGGCAGGACCAG | 10877425 | 10877444 | pos: 2726- | 31.90 | 0.91 |
| | | | | | 2743 | | |
| TAR1604 | 1576 | GAGGCATCTGGGCGGAGGG | 10876838 | 10876858 | pos: 2137- | 12.84 | 0.37 |
| | | | | | 2158 | | |
| TAR1616 | 1577 | GAACAGATGGTGGGAAGAG | 10877907 | 10877927 | pos: 3206- | 11.58 | 0.33 |
| | | | | | 3227 | | |
| N/A | | | | | | 0.00 | 0.00 |
| TAR1647 | 1578 | GTAGTCAGATGGTTTGGGG | 10876631 | 10876651 | pos: 1932- | 26.91 | 0.77 |
| | | | | | 1950 | | |
| TAR1649 | 1579 | ACAGAAAGTCTGTTGGGGG | 10876961 | 10876981 | pos: 2262- | 27.86 | 0.80 |
| | | | | | 2280 | | |
| TAR1651 | 1580 | GTGGACTGAATTTGGTGAA | 10876993 | 10877013 | pos: 2294- | 33.45 | 0.96 |
| | | | | | 2312 | | |
| TAR1653 | 1581 | TAGGACCCAGCAGGGCGTG | 10877345 | 10877365 | pos: 2646- | 26.23 | 0.75 |
| | | | | | 2664 | | |
| TAR1654 | 1582 | GACAGGTAGGACCCAGCAG | 10877351 | 10877371 | pos: 2652- | 29.89 | 0.85 |
| | | | | | 2670 | | |
| TAR1656 | 1583 | GGGGAGGGCAGCATCCGCC | 10877844 | 10877864 | pos: 3145- | 33.10 | 0.95 |
| | | | | | 3163 | | |
| TAR186 | 1584 | GAGGCTGTGTGCTTCTGAG | 10877218 | 10877238 | 0 | 28.64 | 0.82 |
| TAR1577 | 1585 | CTGGAATGGCAGGACCAG | 10877425 | 10877444 | pos: 2726- | 22.25 | 0.64 |
| | | | | | 2743 | | |
| TAR1578 | 1586 | GGAGTGGAGTCTGGCAGA | 10877746 | 10877765 | pos: 3047- | 30.90 | 0.88 |
| | | | | | 3064 | | |
| TAR1618 | 1587 | TAGGAACTGGAATTGAGCT | 10878114 | 10878134 | pos: 3413- | 24.19 | 0.69 |
| | | | | | 3434 | | |
| TAR1648 | 1588 | GAGAGCCTCTGCTGGGGAC | 10876809 | 10876829 | pos: 2110- | 26.65 | 0.76 |
| | | | | | 2128 | | |
| TAR1649 | 1589 | ACAGAAAGTCTGTTGGGGG | 10876961 | 10876981 | pos: 2262- | 27.75 | 0.79 |
| | | | | | 2280 | | |
| TAR1651 | 1590 | GTGGACTGAATTTGGTGAA | 10876993 | 10877013 | pos: 2294- | 19.65 | 0.56 |
| | | | | | 2312 | | |
| TAR1653 | 1591 | TAGGACCCAGCAGGGCGTG | 10877345 | 10877365 | pos: 2646- | 24.60 | 0.70 |
| | | | | | 2664 | | |
| TAR1654 | 1592 | GACAGGTAGGACCCAGCAG | 10877351 | 10877371 | pos: 2652- | 12.21 | 0.35 |
| | | | | | 2670 | | |
| TAR1656 | 1593 | GGGGAGGGCAGCATCCGCC | 10877844 | 10877864 | pos: 3145- | 26.58 | 0.76 |
| | | | | | 3163 | | |
| TAR186 | 1594 | GAGGCTGTGTGCTTCTGAG | 10877218 | 10877238 | 0 | 21.45 | 0.61 |
| TAR1619 | 1595 | GATGGTTAAAGGGGTGGTA | 10878169 | 10878189 | pos: 3468- | 20.65 | 0.59 |
| | | | | | 3489 | | |
| TAR1605 | 1596 | TGGGGAAGCTGAGGGCACG | 10877258 | 10877278 | pos: 2557- | 18.35 | 0.52 |
| | | | | | 2578 | | |
| TAR1608 | 1597 | GGAGCTGCTGCCTGGCTGG | 10877298 | 10877318 | pos: 2597- | 20.00 | 0.57 |
| | | | | | 2618 | | |
| TAR1610 | 1598 | TGGGGGTGAGGATGGGAAC | 10877473 | 10877493 | pos: 2772- | 24.76 | 0.71 |
| | | | | | 2793 | | |
| TAR1612 | 1599 | GCAGCTGGGGAGAGCTGGC | 10877828 | 10877848 | pos: 3127- | 12.50 | 0.36 |
| | | | | | 3148 | | |
| TAR1613 | 1600 | GCTGGGGAGAGCTGGCGGA | 10877831 | 10877851 | pos: 3130- | 25.25 | 0.72 |
| | | | | | 3151 | | |
| TAR1615 | 1601 | GACGGGGGAACAGATGGTG | 10877900 | 10877920 | pos: 3199- | 4.50 | 0.13 |
| | | | | | 3220 | | |
| TAR1617 | 1602 | GTTGGCTGGGAAGGGTGTG | 10877977 | 10877997 | pos: 3276- | 19.60 | 0.56 |
| | | | | | 3297 | | |
| TAR1618 | 1603 | TAGGAACTGGAATTGAGCT | 10878114 | 10878134 | pos: 3413- | 23.92 | 0.68 |
| | | | | | 3434 | | |
| TAR1620 | 1604 | GGTGTGGTTGGGTGCAGCC | 10878256 | 10878276 | pos: 3555- | 20.65 | 0.59 |
| | | | | | 3576 | | |
| TAR1623 | 1605 | GGAGGGGGAGAAGTCAGAG | 10878298 | 10878318 | pos: 3597- | 10.56 | 0.30 |
| | | | | | 3618 | | |
| TAR1623 | 1606 | GGAGGGGGAGAAGTCAGAG | 10878298 | 10878318 | pos: 3597- | 28.45 | 0.81 |
| | | | | | 3618 | | |
| N/A | | | | | | 36.75 | 1.05 |
| TAR1605 | 1607 | TGGGGAAGCTGAGGGCACG | 10877258 | 10877278 | pos: 2557- | 37.40 | 1.07 |
| | | | | | 2578 | | |
| TAR1608 | 1608 | GGAGCTGCTGCCTGGCTGG | 10877298 | 10877318 | pos: 2597- | 12.05 | 0.34 |
| | | | | | 2618 | | |
| TAR1610 | 1609 | TGGGGGTGAGGATGGGAAC | 10877473 | 10877493 | pos: 2772- | 24.67 | 0.70 |
| | | | | | 2793 | | |
| TAR1612 | 1610 | GCAGCTGGGGAGAGCTGGC | 10877828 | 10877848 | pos: 3127- | 17.00 | 0.49 |
| | | | | | 3148 | | |
| TAR1614 | 1611 | GGGGAGAGCTGGCGGATGC | 10877834 | 10877854 | pos: 3133- | 32.84 | 0.94 |
| | | | | | 3154 | | |
| TAR1615 | 1612 | GACGGGGGAACAGATGGTG | 10877900 | 10877920 | pos: 3199- | 25.65 | 0.73 |
| | | | | | 3220 | | |
| TAR1617 | 1613 | GTTGGCTGGGAAGGGTGTG | 10877977 | 10877997 | pos: 3276- | 16.51 | 0.47 |
| | | | | | 3297 | | |
| TAR1618 | 1614 | TAGGAACTGGAATTGAGCT | 10878114 | 10878134 | pos: 3413- | 10.83 | 0.31 |
| | | | | | 3434 | | |
| TAR1620 | 1615 | GGTGTGGTTGGGTGCAGCC | 10878256 | 10878276 | pos: 3555- | 28.15 | 0.80 |
| | | | | | 3576 | | |
| TAR1623 | 1616 | GGAGGGGGAGAAGTCAGAG | 10878298 | 10878318 | pos: 3597- | 27.16 | 0.78 |
| | | | | | 3618 | | |
| N/A | | | | | | 9.58 | 0.27 |
| TAR1605 | 1617 | TGGGGAAGCTGAGGGCACG | 10877258 | 10877278 | pos: 2557- | 17.50 | 0.50 |
| | | | | | 2578 | | |
| TAR1608 | 1618 | GGAGCTGCTGCCTGGCTGG | 10877298 | 10877318 | pos: 2597- | 11.72 | 0.33 |
| | | | | | 2618 | | |
| TAR1611 | 1619 | GAGGATGGGAACAGGAGTC | 10877480 | 10877500 | pos: 2779- | 17.73 | 0.51 |
| | | | | | 2800 | | |
| TAR1612 | 1620 | GCAGCTGGGGAGAGCTGGC | 10877828 | 10877848 | pos: 3127- | 22.01 | 0.63 |
| | | | | | 3148 | | |
| TAR1614 | 1621 | GGGGAGAGCTGGCGGATGC | 10877834 | 10877854 | pos: 3133- | 17.33 | 0.49 |
| | | | | | 3154 | | |
| TAR1615 | 1622 | GACGGGGGAACAGATGGTG | 10877900 | 10877920 | pos: 3199- | 22.72 | 0.65 |
| | | | | | 3220 | | |
| TAR1617 | 1623 | GTTGGCTGGGAAGGGTGTG | 10877977 | 10877997 | pos: 3276- | 22.35 | 0.64 |
| | | | | | 3297 | | |
| TAR1619 | 1624 | GATGGTTAAAGGGGTGGTA | 10878169 | 10878189 | pos: 3468- | 10.31 | 0.29 |
| | | | | | 3489 | | |
| TAR1621 | 1625 | GCAGCCTTAAGTTGCTACG | 10878269 | 10878289 | pos: 3568- | 15.22 | 0.43 |
| | | | | | 3589 | | |
| TAR1624 | 1626 | GGGGGAGAAGTCAGAGGTA | 10878301 | 10878321 | pos: 3600- | 23.79 | 0.68 |
| | | | | | 3621 | | |
| N/A | | | | | | 33.21 | 0.95 |
| TAR1606 | 1627 | GCTGAGGGCACGAGGAGGG | 10877265 | 10877285 | pos: 2564- | 22.36 | 0.6 |
| | | | | | 2585 | | |
| TAR1608 | 1628 | GGAGCTGCTGCCTGGCTGG | 10877298 | 10877318 | pos: 2597- | 28.60 | 0.82 |
| | | | | | 2618 | | |
| TAR1611 | 1629 | GAGGATGGGAACAGGAGTC | 10877480 | 10877500 | pos: 2779- | 12.15 | 0.35 |
| | | | | | 2800 | | |
| TAR1613 | 1630 | GCTGGGGAGAGCTGGCGGA | 10877831 | 10877851 | pos: 3130- | 24.56 | 0.70 |
| | | | | | 3151 | | |
| TAR1614 | 1631 | GGGGAGAGCTGGCGGATGC | 10877834 | 10877854 | pos: 3133- | 25.60 | 0.73 |
| | | | | | 3154 | | |
| TAR1615 | 1632 | GACGGGGGAACAGATGGTG | 10877900 | 10877920 | pos: 3199- | 41.81 | 1.19 |
| | | | | | 3220 | | |
| TAR1617 | 1633 | GTTGGCTGGGAAGGGTGTG | 10877977 | 10877997 | pos: 3276- | 27.81 | 0.79 |
| | | | | | 3297 | | |
| TAR1619 | 1634 | GATGGTTAAAGGGGTGGTA | 10878169 | 10878189 | pos: 3468- | 28.15 | 0.80 |
| | | | | | 3489 | | |
| TAR1621 | 1635 | GCAGCCTTAAGTTGCTACG | 10878269 | 10878289 | pos: 3568- | 8.46 | 0.24 |
| | | | | | 3589 | | |
| TAR1624 | 1636 | GGGGGAGAAGTCAGAGGTA | 10878301 | 10878321 | pos: 3600- | 26.50 | 0.76 |
| | | | | | 3621 | | |
| N/A | | | | | | 32.85 | 0.94 |
| TAR1606 | 1637 | GCTGAGGGCACGAGGAGGG | 10877265 | 10877285 | pos: 2564- | 14.20 | 0.41 |
| | | | | | 2585 | | |
| TAR1608 | 1638 | GGAGCTGCTGCCTGGCTGG | 10877298 | 10877318 | pos: 2597- | 16.08 | 0.46 |
| | | | | | 2618 | | |
| TAR1611 | 1639 | GAGGATGGGAACAGGAGTC | 10877480 | 10877500 | pos: 2779- | 4.22 | 0.12 |
| | | | | | 2800 | | |
| TAR1613 | 1640 | GCTGGGGAGAGCTGGCGGA | 10877831 | 10877851 | pos: 3130- | 17.94 | 0.51 |
| | | | | | 3151 | | |
| TAR1614 | 1641 | GGGGAGAGCTGGCGGATGC | 10877834 | 10877854 | pos: 3133- | 28.57 | 0.82 |
| | | | | | 3154 | | |
| TAR1616 | 1642 | GAACAGATGGTGGGAAGAG | 10877907 | 10877927 | pos: 3206- | 16.87 | 0.48 |
| | | | | | 3227 | | |
| TAR1617 | 1643 | GTTGGCTGGGAAGGGTGTG | 10877977 | 10877997 | pos: 3276- | 22.88 | 0.65 |
| | | | | | 3297 | | |
| TAR1619 | 1644 | GATGGTTAAAGGGGTGGTA | 10878169 | 10878189 | pos: 3468- | 28.86 | 0.82 |
| | | | | | 3489 | | |
| TAR1621 | 1645 | GCAGCCTTAAGTTGCTACG | 10878269 | 10878289 | pos: 3568- | 29.16 | 0.83 |
| | | | | | 3589 | | |
| TAR1624 | 1646 | GGGGGAGAAGTCAGAGGTA | 10878301 | 10878321 | pos: 3600- | 25.10 | 0.72 |
| | | | | | 3621 | | |
| N/A | | | | | | 0.00 | 0.00 |
| TAR1606 | 1647 | GCTGAGGGCACGAGGAGGG | 10877265 | 10877285 | pos: 2564- | 21.90 | 0.63 |
| | | | | | 2585 | | |
| TAR1608 | 1648 | GGAGCTGCTGCCTGGCTGG | 10877298 | 10877318 | pos: 2597- | 29.52 | 0.84 |
| | | | | | 2618 | | |
| TAR1612 | 1649 | GCAGCTGGGGAGAGCTGGC | 10877828 | 10877848 | pos: 3127- | 26.10 | 0.75 |
| | | | | | 3148 | | |
| TAR1613 | 1650 | GCTGGGGAGAGCTGGCGGA | 10877831 | 10877851 | pos: 3130- | 12.44 | 0.36 |
| | | | | | 3151 | | |
| TAR1614 | 1651 | GGGGAGAGCTGGCGGATGC | 10877834 | 10877854 | pos: 3133- | 24.41 | 0.70 |
| | | | | | 3154 | | |
| TAR1616 | 1652 | GAACAGATGGTGGGAAGAG | 10877907 | 10877927 | pos: 3206- | 21.25 | 0.61 |
| | | | | | 3227 | | |
| TAR1618 | 1653 | TAGGAACTGGAATTGAGCT | 10878114 | 10878134 | pos: 3413- | 32.00 | 0.91 |
| | | | | | 3434 | | |
| TAR1619 | 1654 | GATGGTTAAAGGGGTGGTA | 10878169 | 10878189 | pos: 3468- | 29.51 | 0.84 |
| | | | | | 3489 | | |
| TAR1622 | 1655 | GCTACGGGAAAGTGTTGGA | 10878282 | 10878302 | pos: 3581- | 28.33 | 0.81 |
| | | | | | 3602 | | |
| N/A | | | | | | 0.00 | 0.00 |
| TAR1607 | 1656 | AGGGCACGAGGAGGGGCTG | 10877269 | 10877289 | pos: 2568- | 32.64 | 0.93 |
| | | | | | 2589 | | |
| TAR1609 | 1657 | TGGGGATGGGGGTGAGGAT | 10877467 | 10877487 | pos: 2766- | 13.90 | 0.40 |
| | | | | | 2787 | | |
| TAR1612 | 1658 | GCAGCTGGGGAGAGCTGGC | 10877828 | 10877848 | pos: 3127- | 15.55 | 0.44 |
| | | | | | 3148 | | |
| TAR1613 | 1659 | GCTGGGGAGAGCTGGCGGA | 10877831 | 10877851 | pos: 3130- | 4.75 | 0.14 |
| | | | | | 3151 | | |
| TAR1615 | 1660 | GACGGGGGAACAGATGGTG | 10877900 | 10877920 | pos: 3199- | 17.22 | 0.49 |
| | | | | | 3220 | | |
| TAR1616 | 1661 | GAACAGATGGTGGGAAGAG | 10877907 | 10877927 | pos: 3206- | 8.04 | 0.23 |
| | | | | | 3227 | | |
| TAR1618 | 1662 | TAGGAACTGGAATTGAGCT | 10878114 | 10878134 | pos: 3413- | 34.86 | 1.00 |
| | | | | | 3434 | | |
| TAR1619 | 1663 | GATGGTTAAAGGGGTGGTA | 10878169 | 10878189 | pos: 3468- | 35.95 | 1.03 |
| | | | | | 3489 | | |
| TAR1622 | 1664 | GCTACGGGAAAGTGTTGGA | 10878282 | 10878302 | pos: 3581- | 29.30 | 0.84 |
| | | | | | 3602 | | |
| TAR1607 | 1665 | AGGGCACGAGGAGGGGCTG | 10877269 | 10877289 | pos: 2568- | 7.62 | 0.22 |
| | | | | | 2589 | | |
| TAR1609 | 1666 | TGGGGATGGGGGTGAGGAT | 10877467 | 10877487 | pos: 2766- | 9.89 | 0.28 |
| | | | | | 2787 | | |
| TAR1612 | 1667 | GCAGCTGGGGAGAGCTGGC | 10877828 | 10877848 | pos: 3127- | 26.40 | 0.75 |
| | | | | | 3148 | | |
| TAR1613 | 1668 | GCTGGGGAGAGCTGGCGGA | 10877831 | 10877851 | pos: 3130- | 17.83 | 0.51 |
| | | | | | 3151 | | |
| TAR1615 | 1669 | GACGGGGGAACAGATGGTG | 10877900 | 10877920 | pos: 3199- | 4.96 | 0.14 |
| | | | | | 3220 | | |
| TAR1617 | 1670 | GTTGGCTGGGAAGGGTGTG | 10877977 | 10877997 | pos: 3276- | 21.50 | 0.61 |
| | | | | | 3297 | | |
| TAR1618 | 1671 | TAGGAACTGGAATTGAGCT | 10878114 | 10878134 | pos: 3413- | 13.63 | 0.39 |
| | | | | | 3434 | | |
| TAR1620 | 1672 | GGTGTGGTTGGGTGCAGCC | 10878256 | 10878276 | pos: 3555- | 37.65 | 1.08 |
| | | | | | 3576 | | |
| TAR1622 | 1673 | GCTACGGGAAAGTGTTGGA | 10878282 | 10878302 | pos: 3581- | 28.51 | 0.81 |
| | | | | | 3602 | | |
| TAR1557 | 1674 | GAAAATGACAGGTGGGCC | 10876716 | 10876735 | pos: 2015- | 24.07 | 0.69 |
| | | | | | 2035 | | |
| TAR1558 | 1675 | TTGGTGTGGGAGTAGGCA | 10876765 | 10876784 | pos: 2064- | 8.77 | 0.25 |
| | | | | | 2084 | | |
| TAR1561 | 1676 | GGTCAGGGGACGGGGGAA | 10877892 | 10877911 | pos: 3191- | 24.59 | 0.70 |
| | | | | | 3211 | | |
| TAR1562 | 1677 | GGCTGGGAAGGGTGTGCC | 10877980 | 10877999 | pos: 3279- | 11.99 | 0.34 |
| | | | | | 3299 | | |
| TAR1565 | 1678 | GTTGGAGGGGGAGAAGTC | 10878295 | 10878314 | pos: 3594- | 21.60 | 0.62 |
| | | | | | 3614 | | |
| TAR1577 | 1679 | CTGGAATGGCAGGACCAG | 10877425 | 10877444 | pos: 2726- | 11.82 | 0.34 |
| | | | | | 2743 | | |
| TAR1579 | 1680 | GCCAACGGTGTAGCTGCC | 10877965 | 10877984 | pos: 3266- | 14.18 | 0.40 |
| | | | | | 3283 | | |
| TAR1580 | 1681 | GGAGTCTGAGAAAAGGGA | 10878188 | 10878207 | pos: 3489- | 40.20 | 1.15 |
| | | | | | 3506 | | |
| TAR1602 | 1682 | GCAATTTGGTGTGGGAGTA | 10876760 | 10876780 | pos: 2059- | 29.30 | 0.84 |
| | | | | | 2080 | | |
| TAR1604 | 1683 | GAGGCATCTGGGCGGAGGG | 10876838 | 10876858 | pos: 2137- | 30.85 | 0.88 |
| | | | | | 2158 | | |
| N/A | | | | | | 32.65 | 0.93 |
| TAR1557 | 1684 | GAAAATGACAGGTGGGCC | 10876716 | 10876735 | pos: 2015- | 7.9 | 0.23 |
| | | | | | 2035 | | |
| TAR1559 | 1685 | GGGTGTTCAGAAACAGAA | 10877076 | 10877095 | pos: 2375- | 26.60 | 0.76 |
| | | | | | 2395 | | |
| TAR1561 | 1686 | GGTCAGGGGACGGGGGAA | 10877892 | 10877911 | pos: 3191- | 5.05 | 0.14 |
| | | | | | 3211 | | |
| TAR1562 | 1687 | GGCTGGGAAGGGTGTGCC | 10877980 | 10877999 | pos: 3279- | 26.34 | 0.75 |
| | | | | | 3299 | | |
| TAR1565 | 1688 | GTTGGAGGGGGAGAAGTC | 10878295 | 10878314 | pos: 3594- | 12.40 | 0.35 |
| | | | | | 3614 | | |
| TAR1577 | 1689 | CTGGAATGGCAGGACCAG | 10877425 | 10877444 | pos: 2726- | 23.74 | 0.68 |
| | | | | | 2743 | | |
| TAR1579 | 1690 | GCCAACGGTGTAGCTGCC | 10877965 | 10877984 | pos: 3266- | 12.75 | 0.36 |
| | | | | | 3283 | | |
| TAR1582 | 1691 | GCATGAGACTTTGAGGTG | 10878379 | 10878398 | pos: 3680- | 27.75 | 0.79 |
| | | | | | 3697 | | |
| TAR1603 | 1692 | GAAGTCCCCAGCAGAGGCT | 10876806 | 10876826 | pos: 2105- | 30.80 | 0.88 |
| | | | | | 2126 | | |
| N/A | | | | | | 12.16 | 0.35 |
| TAR1557 | 1693 | GAAAATGACAGGTGGGCC | 10876716 | 10876735 | pos: 2015- | 16.77 | 0.48 |
| | | | | | 2035 | | |
| TAR1559 | 1694 | GGGTGTTCAGAAACAGAA | 10877076 | 10877095 | pos: 2375- | 20.01 | 0.57 |
| | | | | | 2395 | | |
| TAR1561 | 1695 | GGTCAGGGGACGGGGGAA | 10877892 | 10877911 | pos: 3191- | 24.98 | 0.71 |
| | | | | | 3211 | | |
| TAR1563 | 1696 | GCCGGTGTGGTTGGGTGC | 10878253 | 10878272 | pos: 3552- | 21.16 | 0.60 |
| | | | | | 3572 | | |
| TAR1566 | 1697 | GAGGACCCAGCAGGAATC | 10878401 | 10878420 | pos: 3700- | 22.96 | 0.66 |
| | | | | | 3720 | | |
| TAR1577 | 1698 | CTGGAATGGCAGGACCAG | 10877425 | 10877444 | pos: 2726- | 25.27 | 0.72 |
| | | | | | 2743 | | |
| TAR1579 | 1699 | GCCAACGGTGTAGCTGCC | 10877965 | 10877984 | pos: 3266 | 24.27 | 0.69 |
| | | | | | 3283 | | |
| TAR1600 | 1700 | GATACTGTTGACTGTAGAA | 10876423 | 10876443 | pos: 1722- | 29.75 | 0.85 |
| | | | | | 1743 | | |
| TAR1603 | 1701 | GAAGTCCCCAGCAGAGGCT | 10876806 | 10876826 | pos: 2105- | 28.98 | 0.83 |
| | | | | | 2126 | | |
| N/A | | | | | | 26.30 | 0.75 |
| TAR1557 | 1702 | GAAAATGACAGGTGGGCC | 10876716 | 10876735 | pos: 2015- | 3.68 | 0.11 |
| | | | | | 2035 | | |
| TAR1559 | 1703 | GGGTGTTCAGAAACAGAA | 10877076 | 10877095 | pos: 2375- | 17.06 | 0.49 |
| | | | | | 2395 | | |
| TAR1561 | 1704 | GGTCAGGGGACGGGGGAA | 10877892 | 10877911 | pos: 3191- | 20.30 | 0.58 |
| | | | | | 3211 | | |
| TAR1563 | 1705 | GCCGGTGTGGTTGGGTGC | 10878253 | 10878272 | pos: 3552- | 5.44 | 0.16 |
| | | | | | 3572 | | |
| TAR1566 | 1706 | GAGGACCCAGCAGGAATC | 10878401 | 10878420 | pos: 3700- | 22.02 | 0.63 |
| | | | | | 3720 | | |
| TAR1577 | 1707 | CTGGAATGGCAGGACCAG | 10877425 | 10877444 | pos: 2726- | 23.42 | 0.67 |
| | | | | | 2743 | | |
| TAR1579 | 1708 | GCCAACGGTGTAGCTGCC | 10877965 | 10877984 | pos: 3266- | 15.72 | 0.45 |
| | | | | | 3283 | | |
| TAR1600 | 1709 | GATACTGTTGACTGTAGAA | 10876423 | 10876443 | pos: 1722- | 27.55 | 0.79 |
| | | | | | 1743 | | |
| TAR1603 | 1710 | GAAGTCCCCAGCAGAGGCT | 10876806 | 10876826 | pos: 2105- | 27.10 | 0.77 |
| | | | | | 2126 | | |
| N/A | | | | | | 28.65 | 0.82 |
| TAR1557 | 1711 | GAAAATGACAGGTGGGCC | 10876716 | 10876735 | pos: 2015- | 23.66 | 0.68 |
| | | | | | 2035 | | |
| TAR1559 | 1712 | GGGTGTTCAGAAACAGAA | 10877076 | 10877095 | pos: 2375- | 10.95 | 0.31 |
| | | | | | 2395 | | |
| TAR1561 | 1713 | GGTCAGGGGACGGGGGAA | 10877892 | 10877911 | pos: 3191- | 12.36 | 0.35 |
| | | | | | 3211 | | |
| TAR1563 | 1714 | GCCGGTGTGGTTGGGTGC | 10878253 | 10878272 | pos: 3552- | 22.92 | 0.65 |
| | | | | | 3572 | | |
| TAR1566 | 1715 | GAGGACCCAGCAGGAATC | 10878401 | 10878420 | pos: 3700- | 6.68 | 0.19 |
| | | | | | 3720 | | |
| TAR1578 | 1716 | GGAGTGGAGTCTGGCAGA | 10877746 | 10877765 | pos: 3047- | 26.03 | 0.74 |
| | | | | | 3064 | | |
| TAR1580 | 1717 | GGAGTCTGAGAAAAGGGA | 10878188 | 10878207 | pos: 3489- | 24.59 | 0.70 |
| | | | | | 3506 | | |
| TAR1600 | 1718 | GATACTGTTGACTGTAGAA | 10876423 | 10876443 | pos: 1722- | 29.09 | 0.83 |
| | | | | | 1743 | | |
| TAR1604 | 1719 | GAGGCATCTGGGCGGAGGG | 10876838 | 10876858 | pos: 2137- | 32.23 | 0.92 |
| | | | | | 2158 | | |
| TAR1557 | 1720 | GAAAATGACAGGTGGGCC | 10876716 | 10876735 | pos: 2015- | 13.60 | 0.39 |
| | | | | | 2035 | | |
| TAR1560 | 1721 | GGGGAAGCTGAGGGCACG | 10877259 | 10877278 | pos: 2558- | 24.88 | 0.71 |
| | | | | | 2578 | | |
| TAR1562 | 1722 | GGCTGGGAAGGGTGTGCC | 10877980 | 10877999 | pos: 3279- | 8.00 | 0.23 |
| | | | | | 3299 | | |
| TAR1564 | 1723 | TAAGTTGCTACGGGAAAG | 10878276 | 10878295 | pos: 3575- | 32.96 | 0.94 |
| | | | | | 3595 | | |
| TAR1576 | 1724 | GACAGGTAGGACCCAGCA | 10877352 | 10877371 | pos: 2653- | 20.25 | 0.58 |
| | | | | | 2670 | | |
| TAR1578 | 1725 | GGAGTGGAGTCTGGCAGA | 10877746 | 10877765 | pos: 3047- | 7.22 | 0.21 |
| | | | | | 3064 | | |
| TAR1580 | 1726 | GGAGTCTGAGAAAAGGGA | 10878188 | 10878207 | pos: 3489- | 26.65 | 0.76 |
| | | | | | 3506 | | |
| TAR1601 | 1727 | GACTGTAGAAGGCTCTGAG | 10876432 | 10876452 | pos: 1731- | 26.80 | 0.77 |
| | | | | | 1752 | | |
| TAR1604 | 1728 | GAGGCATCTGGGCGGAGGG | 10876838 | 10876858 | pos: 2137- | 30.70 | 0.88 |
| | | | | | 2158 | | |
| N/A | | | | | | 0.00 | 0.00 |
| TAR1558 | 1729 | TTGGTGTGGGAGTAGGCA | 10876765 | 10876784 | pos: 2064- | 10.80 | 0.31 |
| | | | | | 2084 | | |
| TAR1560 | 1730 | GGGGAAGCTGAGGGCACG | 10877259 | 10877278 | pos: 2558- | 12.60 | 0.36 |
| | | | | | 2578 | | |
| TAR1562 | 1731 | GGCTGGGAAGGGTGTGCC | 10877980 | 10877999 | pos: 3279- | 24.04 | 0.69 |
| | | | | | 3299 | | |
| TAR1564 | 1732 | TAAGTTGCTACGGGAAAG | 10878276 | 10878295 | pos: 3575- | 3.55 | 0.10 |
| | | | | | 3595 | | |
| TAR1576 | 1733 | GACAGGTAGGACCCAGCA | 10877352 | 10877371 | pos: 2653- | 36.43 | 1.04 |
| | | | | | 2670 | | |
| TAR1579 | 1734 | GCCAACGGTGTAGCTGCC | 10877965 | 10877984 | pos: 3266- | 25.42 | 0.73 |
| | | | | | 3283 | | |
| TAR1580 | 1735 | GGAGTCTGAGAAAAGGGA | 10878188 | 10878207 | pos: 3489- | 8.88 | 0.25 |
| | | | | | 3506 | | |
| TAR1601 | 1736 | GACTGTAGAAGGCTCTGAG | 10876432 | 10876452 | pos: 1731- | 30.95 | 0.88 |
| | | | | | 1752 | | |
| TAR1604 | 1737 | GAGGCATCTGGGCGGAGGG | 10876838 | 10876858 | pos: 2137- | 32.45 | 0.93 |
| | | | | | 2158 | | |
| TAR1558 | 1738 | TTGGTGTGGGAGTAGGCA | 10876765 | 10876784 | pos: 2064- | 26.04 | 0.74 |
| | | | | | 2084 | | |
| TAR1560 | 1739 | GGGGAAGCTGAGGGCACG | 10877259 | 10877278 | pos: 2558- | 17.32 | 0.49 |
| | | | | | 2578 | | |
| TAR1562 | 1740 | GGCTGGGAAGGGTGTGCC | 10877980 | 10877999 | pos: 3279- | 9.30 | 0.27 |
| | | | | | 3299 | | |
| TAR1565 | 1741 | GTTGGAGGGGGAGAAGTC | 10878295 | 10878314 | pos: 3594- | 10.81 | 0.31 |
| | | | | | 3614 | | |
| TAR1576 | 1742 | GACAGGTAGGACCCAGCA | 10877352 | 10877371 | pos: 2653- | 18.43 | 0.53 |
| | | | | | 2670 | | |
| TAR1579 | 1743 | GCCAACGGTGTAGCTGCC | 10877965 | 10877984 | pos: 3266- | 40.50 | 1.16 |
| | | | | | 3283 | | |
| TAR1580 | 1744 | GGAGTCTGAGAAAAGGGA | 10878188 | 10878207 | pos: 3489- | 24.14 | 0.69 |
| | | | | | 3506 | | |
| TAR1601 | 1745 | GACTGTAGAAGGCTCTGAG | 10876432 | 10876452 | pos: 1731- | 28.95 | 0.83 |
| | | | | | 1752 | | |
| TAR1604 | 1746 | GAGGCATCTGGGCGGAGGG | 10876838 | 10876858 | pos: 2137- | 30.05 | 0.86 |
| | | | | | 2158 | | |

| | Target | | SEQ ID |
|---|---|---|---|
| Name | Sequence | ZFP Protein Sequence | NO: |
| CMV:ZFP | TGGGGAA | SRPGERPFQCRICMRNFSKKIDLVRHTRTHTGEKPFQCRICMRNFS | 1980 |
| 660- | GCTGAGG | LKGHLTRHLRTHTGSQKPFQCRICMRNFSRQDNLGRHLRTHTGE | |
| OFF(mil) | GCACG | KPFQCRICMRNFSVSNTLTRHLKTHTGGGGSQKPFQCRICMRNFS | |
| | | QSAHLKRHLRTHTGEKPFQCRICMRNFSRSDHLSLHLKTHLRGS | |
| CMV:ZFP | GGAGCTG | SRPGERPFQCRICMRNFSRKEHLSIHLRTHTGEKPFQCRICMRNFS | 1981 |
| 669- | CTGCCTG | DKSHLTRHLKTHTGGGGSQKPFQCRICMRNFSDGSTLRRHTRTH | |
| OFF(mil) | GCTGG | TGEKPFQCRICMRNFSLSQTLKRHLRTHTGSQKPFQCRICMRNFS | |
| | | QRSDLTRHLRTHTGEKPFQCRICMRNFSQSAHLGRHLKTHLRGS | |
| CMV:ZFP | TGGGGGT | SRPGERPFQCRICMRNFSHRTNLIAHTRTHTGEKPFQCRICMRNFS | 1982 |
| 678- | GAGGATG | RREHLVRHLRTHTGSQKPFQCRICMRNFSVPHNLQRHLRTHTGE | |
| OFF(mil) | GGAAC | KPFQCRICMRNFSRVDNLGRHLKTHTGGGGSQKPFQCRICMRNF | |
| | | SRKHHLGRHTRTHTGEKPFQCRICMRNFSRREHLTIHLRTHLRGS | |
| CMV:ZFP | GCAGCTG | SRPGERPFQCRICMRNFSSPSKLARHTRTHTGEKPFQCRICMRNFS | 1983 |
| 687- | GGGAGAG | QRSDLTRHLRTHTGGGGSQKPFQCRICMRNFSRQANLTRHTRTH | |
| OFF(mil) | CTGGC | TGEKPFQCRICMRNFSRQEHLVRHLRTHTGSQKPFQCRICMRNFS | |
| | | QRSDLTRHLRTHTGEKPFQCRICMRNFSQGGTLRRHLKTHLRGS | |
| CMV:ZFP | GCTGGGG | SRPGERPFQCRICMRNFSQSAHLKRHLRTHTGEKPFQCRICMRNF | 1984 |
| 695- | AGAGCTG | SESGHLKRHLKTHTGGGGSQKPFQCRICMRNFSFHSYLQKHLRT | |
| OFF(mil) | GCGGA | HTGEKPFQCRICMRNFSRRDNLLRHLKTHTGSQKPFQCRICMRNF | |
| | | SKKDHLHRHTRTHTGEKPFQCRICMRNFSLSQTLNRHLRTHLRGS | |
| CMV:ZFP | GACGGGG | SRPGERPFQCRICMRNFSRTFVLKRHTRTHTGEKPFQCRICMRNFS | 1985 |
| 704- | GAACAGA | RNDALAVHLRTHTGSQKPFQCRICMRNFSQRRYLVEHTRTHTGE | |
| OFF(mil) | TGGTG | KPFQCRICMRNFSQQTNLARHLRTHTGGGGSQKPFQCRICMRNFS | |
| | | RREHLVRHLRTHTGEKPFQCRICMRNFSDPSNLQRHLKTHLRGS | |
| CMV:ZFP | GTTGGCT | SRPGERPFQCRICMRNFSRREVLENHLRTHTGEKPFQCRICMRNFS | 1986 |
| 715- | GGGAAGG | IKHHLGRHLKTHTGGGGSQKPFQCRICMRNFSQQTNLTRHLRTH | |
| OFF(mil) | GTGTG | TGEKPFQCRICMRNFSRSDHLSLHLKTHTGSQKPFQCRICMRNFS | |
| | | VPSKLLRHTRTHTGEKPFQCRICMRNFSTNSSLTRHLRTHLRGS | |
| CMV:ZFP | TAGGAAC | SRPGERPFQCRICMRNFSTNSMLARHTRTHTGEKPFQCRICMRNF | 1987 |
| 723- | TGGAATT | SQNEHLKVHLRTHTGGGGSQKPFQCRICMRNFSQGGNLTRHLRT | |
| OFF(mil) | GAGCT | HTGEKPFQCRICMRNFSRKMTLMAHLKTHTGSQKPFQCRICMRN | |
| | | FSQQTNLTRHLRTHTGEKPFQCRICMRNFSRIDHLAGHLKTHLRG | |
| | | S | |
| CMV:ZFP | GGTGTGG | SRPGERPFQCRICMRNFSDLSTLRRHTRTHTGEKPFQCRICMRNFS | 1988 |
| 733- | TTGGGTG | QSTTLKRHLRTHTGGGGSQKPFQCRICMRNFSRREHLVRHLRTHT | |
| OFF(mil) | CAGCC | GEKPFQCRICMRNFSINHSLRRHLKTHTGSQKPFQCRICMRNFSRR | |
| | | EVLENHLRTHTGEKPFQCRICMRNFSIRHHLKRHLKTHLRGS | |
| CMV:ZFP | GGAGGGG | SRPGERPFQCRICMRNFSRQDNLGRHLRTHTGEKPFQCRICMRNF | 1989 |
| 741- | GAGAAGT | STNHWLLIHLKTHTGGGGSQKPFQCRICMRNFSQATNLARHLRT | |
| OFF(mil) | CAGAG | HTGEKPFQCRICMRNFSQNSHLRRHLKTHTGSQKPFQCRICMRNF | |
| | | SRREHLMRHLRTHTGEKPFQCRICMRNFSQNSHLRRHLKTHLRG | |
| | | S | |
| CMV:ZFP | TGGGGAA | SRPGERPFQCRICMRNFSKKIDLVRHTRTHTGEKPFQCRICMRNFS | 1990 |
| 661- | GCTGAGG | LKGHLTRHLRTHTGSQKPFQCRICMRNFSRQDNLGRHLRTHTGE | |
| OFF(mil) | GCACG | KPFQCRICMRNFSVSNTLSRHLKTHTGGGGSQKPFQCRICMRNFS | |
| | | QSAHLKRHLRTHTGEKPFQCRICMRNFSRSDHLSLHLKTHLRGS | |
| CMV:ZFP | GGAGCTG | SRPGERPFQCRICMRNFSRKEHLSIHLRTHTGEKPFQCRICMRNFS | 1991 |
| 670- | CTGCCTG | DKSHLTRHLKTHTGGGGSQKPFQCRICMRNFSDGSTLRRHTRTH | |
| OFF(mil) | GCTGG | TGEKPFQCRICMRNFSLSQTLKRHLRTHTGSQKPFQCRICMRNFS | |
| | | VEASLKRHLRTHTGEKPFQCRICMRNFSQTTHLSRHLKTHLRGS | |
| CMV:ZFP | TGGGGGT | SRPGERPFQCRICMRNFSHRTNLIAHTRTHTGEKPFQCRICMRNFS | 1992 |
| 679- | GAGGATG | RREHLVRHLRTHTGSQKPFQCRICMRNFSVNHNLRRHLRTHTGE | |
| OFF(mil) | GGAAC | KPFQCRICMRNFSRQDNLQRHLKTHTGGGGSQKPFQCRICMRNF | |
| | | SRKHHLGRHTRTHTGEKPFQCRICMRNFSRREHLTIHLRTHLRGS | |
| CMV:ZFP | GCAGCTG | SRPGERPFQCRICMRNFSAPSKLARHTRTHTGEKPFQCRICMRNFS | 1993 |
| 688- | GGGAGAG | VKHSLQRHLRTHTGGGGSQKPFQCRICMRNFSRQANLTRHTRTH | |
| OFF(mil) | CTGGC | TGEKPFQCRICMRNFSRQEHLVRHLRTHTGSQKPFQCRICMRNFS | |
| | | LRDSLKRHLRTHTGEKPFQCRICMRNFSQTATLKRHLKTHLRGS | |
| CMV:ZFP | GGGGAGA | SRPGERPFQCRICMRNFSRSRNLLLHTRTHTGEKPFQCRICMRNFS | 1994 |
| 696- | GCTGGCG | QSAHLKRHLRTHTGSQKPFQCRICMRNFSSPSKLARHTRTHTGEK | |
| OFF(mil) | GATGC | PFQCRICMRNFSVKETLTRHLRTHTGGGGSQKPFQCRICMRNFSR | |
| | | PHNLLRHTRTHTGEKPFQCRICMRNFSRREHLVRHLRTHLRGS | |
| CMV:ZFP | GACGGGG | SRPGERPFQCRICMRNFSRTFVLKRHTRTHTGEKPFQCRICMRNFS | 1995 |
| 705- | GAACAGA | RNDALAVHLRTHTGSQKPFQCRICMRNFSKRFNLMQHTRTHTGE | |
| OFF(mil) | TGGTG | KPFQCRICMRNFSQTQNLTRHLRTHTGGGGSQKPFQCRICMRNFS | |
| | | RREHLVRHLRTHTGEKPFQCRICMRNFSDMGNLGRHLKTHLRGS | |
| CMV:ZFP | GTTGGCT | SRPGERPFQCRICMRNFSRNFILQRHTRTHTGEKPFQCRICMRNFS | 1996 |
| 716- | GGGAAGG | EAHHLSRHLRTHTGGGGSQKPFQCRICMRNFSQQTNLTRHLRTH | |
| OFF(mil) | GTGTG | TGEKPFQCRICMRNFSRSDHLSLHLKTHTGSQKPFQCRICMRNFS | |
| | | APSKLKRHTRTHTGEKPFQCRICMRNFSTNSSLTRHLRTHLRGS | |
| CMV:ZFP | TAGGAAC | SRPGERPFQCRICMRNFSHHTSLVRHTRTHTGEKPFQCRICMRNFS | 1997 |
| 725- | TGGAATT | QREHLKVHLRTHTGGGGSQKPFQCRICMRNFSQRGNLARHLRTH | |
| OFF(mil) | GAGCT | TGEKPFQCRICMRNFSRNHSLRMHLKTHTGSQKPFQCRICMRNFS | |
| | | QQTNLTRHLRTHTGEKPFQCRICMRNFSRIDHLAGHLKTHLRGS | |
| CMV:ZFP | GGTGTGG | SRPGERPFQCRICMRNFSDPSTLRRHTRTHTGEKPFQCRICMRNFS | 1998 |
| 734- | TTGGGTG | QSTTLKRHLRTHTGGGGSQKPFQCRICMRNFSRREHLVRHLRTHT | |
| OFF(mil) | CAGCC | GEKPFQCRICMRNFSINHSLRRHLKTHTGSQKPFQCRICMRNFSRR | |
| | | EVLENHLRTHTGEKPFQCRICMRNFSIRHHLKRHLKTHLRGS | |
| CMV:ZFP | GGAGGGG | SRPGERPFQCRICMRNFSRADNLGRHLRTHTGEKPFQCRICMRNF | 1999 |
| 743- | GAGAAGT | STKQWTLGHLKTHTGGGGSQKPFQCRICMRNFSQQTNLTRHLRT | |
| OFF(mil) | CAGAG | HTGEKPFQCRICMRNFSQTTHLSRHLKTHTGSQKPFQCRICMRNF | |
| | | SKKDHLHRHTRTHTGEKPFQCRICMRNFSQSAHLKRHLRTHLRG | |
| | | S | |
| CMV:ZFP | TGGGGAA | SRPGERPFQCRICMRNFSKKIDLVRHTRTHTGEKPFQCRICMRNFS | 2000 |
| 662- | GCTGAGG | LKGHLTRHLRTHTGSQKPFQCRICMRNFSRQDNLGRHLRTHTGE | |
| OFF(mil) | GCACG | KPFQCRICMRNFSLKHDLRRHLKTHTGGGGSQKPFQCRICMRNFS | |
| | | QSAHLKRHLRTHTGEKPFQCRICMRNFSRSDHLSLHLKTHLRGS | |
| CMV:ZFP | GGAGCTG | SRPGERPFQCRICMRNFSRKEHLSIHLRTHTGEKPFQCRICMRNFS | 2001 |
| 671- | CTGCCTG | DKSHLTRHLKTHTGGGGSQKPFQCRICMRNFSDPSTLRRHTRTHT | |
| OFF(mil) | GCTGG | GEKPFQCRICMRNFSLRDSLKRHLRTHTGSQKPFQCRICMRNFSQ | |
| | | RSDLTRHLRTHTGEKPFQCRICMRNFSQSAHLGRHLKTHLRGS | |
| CMV:ZFP | GAGGATG | SRPGERPFQCRICMRNFSDHSSLKRHLRTHTGEKPFQCRICMRNFS | 2002 |
| 681- | GGAACAG | QSPHLQRHLKTHTGGGGSQKPFQCRICMRNFSHRTNLIAHTRTHT | |
| OFF(mil) | GAGTC | GEKPFQCRICMRNFSRREHLVRHLRTHTGSQKPFQCRICMRNFSV | |
| | | NHNLRRHLRTHTGEKPFQCRICMRNFSRQDNLQRHLKTHLRGS | |
| CMV:ZFP | GCAGCTG | SRPGERPFQCRICMRNFSAPSKLARHTRTHTGEKPFQCRICMRNFS | 2003 |
| 689- | GGGAGAG | VKHSLQRHLRTHTGGGGSQKPFQCRICMRNFSKLTNLARHTRTH | |
| OFF(mil) | CTGGC | TGEKPFQCRICMRNFSRREHLVRHLRTHTGSQKPFQCRICMRNFS | |
| | | TKQVLDRHTRTHTGEKPFQCRICMRNFSQSTTLKRHLRTHLRGS | |
| CMV:ZFP | GGGGAGA | SRPGERPFQCRICMRNFSRSRNLLLHTRTHTGEKPFQCRICMRNFS | 2004 |
| 697- | GCTGGCG | QSAHLKRHLRTHTGSQKPFQCRICMRNFSSPSKLARHTRTHTGEK | |
| OFF(mil) | GATGC | PFQCRICMRNFSVKETLTRHLRTHTGGGGSQKPFQCRICMRNFSR | |
| | | NTNLTRHTRTHTGEKPFQCRICMRNFSRREHLVRHLRTHLRGS | |
| CMV:ZFP | GACGGGG | SRPGERPFQCRICMRNFSRGTVLRRHTRTHTGEKPFQCRICMRNF | 2005 |
| 706- | GAACAGA | SRNDALAVHLRTHTGSQKPFQCRICMRNFSKRFNLMQHTRTHTG | |
| OFF(mil) | TGGTG | EKPFQCRICMRNFSQTQNLTRHLRTHTGGGGSQKPFQCRICMRNF | |
| | | SRREHLVRHLRTHTGEKPFQCRICMRNFSDGGNLGRHLKTHLRG | |
| | | S | |
| CMV:ZFP | GTTGGCT | SRPGERPFQCRICMRNFSRNFILQRHTRTHTGEKPFQCRICMRNFS | 2006 |
| 717- | GGGAAGG | EAHHLSRHLRTHTGGGGSQKPFQCRICMRNFSQAVNLARHLRTH | |
| OFF(mil) | GTGTG | TGEKPFQCRICMRNFSRSDHLSLHLKTHTGSQKPFQCRICMRNFS | |
| | | VPSKLLRHTRTHTGEKPFQCRICMRNFSTNSSLTRHLRTHLRGS | |
| CMV:ZFP | GATGGTT | SRPGERPFQCRICMRNFSQRSSLVRHLRTHTGEKPFQCRICMRNFS | 2007 |
| 726- | AAAGGGG | RTVALNRHLKTHTGSQKPFQCRICMRNFSKGDHLRRHTRTHTGE | |
| OFF(mil) | TGGTA | KPFQCRICMRNFSQRCNLLTHLRTHTGGGGSQKPFQCRICMRNFS | |
| | | LPHHLKRHLRTHTGEKPFQCRICMRNFSISHNLARHLKTHLRGS | |
| CMV:ZFP | GCAGCCT | SRPGERPFQCRICMRNFSRNITLVRHTRTHTGEKPFQCRICMRNFS | 2008 |
| 735- | TAAGTTG | VKETLVRHLRTHTGSQKPFQCRICMRNFSHKSSLTRHLRTHTGEK | |
| OFF(mil) | CTACG | PFQCRICMRNFSQSGNLKGHLKTHTGGGGSQKPFQCRICMRNFSD | |
| | | SSVLRRHLRTHTGEKPFQCRICMRNFSQGGTLRRHLKTHLRGS | |
| CMV:ZFP | GGGGGAG | SRPGERPFQCRICMRNFSQSASLRRHLRTHTGEKPFQCRICMRNFS | 2009 |
| 744- | AAGTCAG | RRDNLTRHLKTHTGGGGSQKPFQCRICMRNFSDHSSLKRHLRTH | |
| OFF(mil) | AGGTA | TGEKPFQCRICMRNFSQHPNLTRHLKTHTGSQKPFQCRICMRNFS | |
| | | QSAHLKRHLRTHTGEKPFQCRICMRNFSRTEHLARHLKTHLRGS | |
| CMV:ZFP | GCTGAGG | SRPGERPFQCRICMRNFSRREHLVRHLRTHTGEKPFQCRICMRNF | 2010 |
| 663- | GCACGAG | SQTTHLRRHLKTHTGGGGSQKPFQCRICMRNFSKKIDLVRHTRTH | |
| OFF(mil) | GAGGG | TGEKPFQCRICMRNFSLKGHLTRHLRTHTGSQKPFQCRICMRNFS | |
| | | RQDNLGRHLRTHTGEKPFQCRICMRNFSLKHDLRRHLKTHLRGS | |
| CMV:ZFP | GGAGCTG | SRPGERPFQCRICMRNFSRREHLTIHLRTHTGEKPFQCRICMRNFS | 2011 |
| 672- | CTGCCTG | EKSHLTRHLKTHTGGGGSQKPFQCRICMRNFSDGSTLRRHTRTHT | |
| OFF(mil) | GCTGG | GEKPFQCRICMRNFSLSQTLKRHLRTHTGSQKPFQCRICMRNFSV | |
| | | KHSLQRHLRTHTGEKPFQCRICMRNFSQTTHLSRHLKTHLRGS | |
| CMV:ZFP | GAGGATG | SRPGERPFQCRICMRNFSDRSSLKRHLRTHTGEKPFQCRICMRNFS | 2012 |
| 682- | GGAACAG | QTAHLHRHLKTHTGGGGSQKPFQCRICMRNFSHRTNLIAHTRTH | |
| OFF(mil) | GAGTC | TGEKPFQCRICMRNFSRREHLVRHLRTHTGSQKPFQCRICMRNFS | |
| | | VPHNLQRHLRTHTGEKPFQCRICMRNFSRVDNLGRHLKTHLRGS | |
| CMV:ZFP | GCTGGGG | SRPGERPFQCRICMRNFSQSAHLKRHLRTHTGEKPFQCRICMRNF | 2013 |
| 690- | AGAGCTG | SESGHLRRHLKTHTGGGGSQKPFQCRICMRNFSFHSYLQKHLRTH | |
| OFF(mil) | GCGGA | TGEKPFQCRICMRNFSRRDNLLRHLKTHTGSQKPFQCRICMRNFS | |
| | | KKDHLHRHTRTHTGEKPFQCRICMRNFSLSQTLNRHLRTHLRGS | |
| CMV:ZFP | GGGGAGA | SRPGERPFQCRICMRNFSRTRNLVMHTRTHTGEKPFQCRICMRNF | 2014 |
| 699- | GCTGGCG | SQSAHLKRHLRTHTGSQKPFQCRICMRNFSSPSKLARHTRTHTGE | |
| OFF(mil) | GATGC | KPFQCRICMRNFSVKETLTRHLRTHTGGGGSQKPFQCRICMRNFS | |
| | | ROANLVRHTRTHTGEKPFQCRICMRNFSRQEHLVRHLRTHLRGS | |
| CMV:ZFP | GACGGGG | SRPGERPFQCRICMRNFSRGTVLRRHTRTHTGEKPFQCRICMRNF | 2015 |
| 707- | GAACAGA | SRNDALAVHLRTHTGSQKPFQCRICMRNFSTTFNLRVHTRTHTGE | |
| OFF(mil) | TGGTG | KPFQCRICMRNFSQTQNLTRHLRTHTGGGGSQKPFQCRICMRNFS | |
| | | RREHLVRHLRTHTGEKPFQCRICMRNFSDPSNLQRHLKTHLRGS | |
| CMV:ZFP | GTTGGCT | SRPGERPFQCRICMRNFSRRFILSRHTRTHTGEKPFQCRICMRNFS | 2016 |
| 718- | GGGAAGG | EAHHLSRHLRTHTGGGGSQKPFQCRICMRNFSQKVNLARHLRTH | |
| OFF(mil) | GTGTG | TGEKPFQCRICMRNFSRSDHLSLHLKTHTGSQKPFQCRICMRNFS | |
| | | VPSKLLRHTRTHTGEKPFQCRICMRNFSTNSSLTRHLRTHLRGS | |
| CMV:ZFP | GATGGTT | SRPGERPFQCRICMRNFSQRSSLVRHLRTHTGEKPFQCRICMRNFS | 2017 |
| 727- | AAAGGGG | RTVALNRHLKTHTGSQKPFQCRICMRNFSKKDHLHRHTRTHTGE | |
| OFF(mil) | TGGTA | KPFQCRICMRNFSQKENLQVHLRTHTGGGGSQKPFQCRICMRNFS | |
| | | LPHHLKRHLRTHTGEKPFQCRICMRNFSISHNLARHLKTHLRGS | |
| CMV:ZFP | GCAGCCT | SRPGERPFQCRICMRNFSRGITLTRHTRTHTGEKPFQCRICMRNFS | 2018 |
| 736- | TAAGTTG | VRETLVRHLRTHTGSQKPFQCRICMRNFSHKSSLTRHLRTHTGEK | |
| OFF(mil) | CTACG | PFQCRICMRNFSQQGNLQLHLKTHTGGGGSQKPFQCRICMRNFS | |
| | | DLSTLRRHTRTHTGEKPFQCRICMRNFSQSTTLKRHLRTHLRGS | |
| CMV:ZFP | GGGGGAG | SRPGERPFQCRICMRNFSQSASLRRHLRTHTGEKPFQCRICMRNFS | 2019 |
| 745- | AAGTCAG | RRDNLTRHLKTHTGGGGSQKPFQCRICMRNFSDPSSLKRHLRTHT | |
| OFF(mil) | AGGTA | GEKPFQCRICMRNFSQHPNLTRHLKTHTGSQKPFQCRICMRNFSQ | |
| | | SAHLKRHLRTHTGEKPFQCRICMRNFSRDDKLAGHLKTHLRGS | |
| CMV:ZFP | GCTGAGG | SRPGERPFQCRICMRNFSKKDHLHRHTRTHTGEKPFQCRICMRNF | 2020 |
| 664- | GCACGAG | SQSAHLKRHLRTHTGGGGSQKPFQCRICMRNFSKKIDLVRHTRTH | |
| OFF(mil) | GAGGG | TGEKPFQCRICMRNFSLKGHLTRHLRTHTGSQKPFQCRICMRNFS | |
| | | RQDNLGRHLRTHTGEKPFQCRICMRNFSVSNTLSRHLKTHLRGS | |
| CMV:ZFP | GGAGCTG | SRPGERPFQCRICMRNFSRKEHLSIHLRTHTGEKPFQCRICMRNFS | 2021 |
| 673- | CTGCCTG | DKSHLTRHLKTHTGGGGSQKPFQCRICMRNFSDLSTLRRHTRTHT | |
| OFF(mil) | GCTGG | GEKPFQCRICMRNFSLSQTLKRHLRTHTGSQKPFQCRICMRNFSV | |
| | | EASLKRHLRTHTGEKPFQCRICMRNFSQTTHLSRHLKTHLRGS | |
| CMV:ZFP | GAGGATG | SRPGERPFQCRICMRNFSDHSSLKRHLRTHTGEKPFQCRICMRNFS | 2022 |
| 683- | GGAACAG | QSPHLQRHLKTHTGGGGSQKPFQCRICMRNFSHRTNLIAHTRTHT | |
| OFF(mil) | GAGTC | GEKPFQCRICMRNFSRREHLVRHLRTHTGSQKPFQCRICMRNFSV | |
| | | NHNLTRHLRTHTGEKPFQCRICMRNFSRRDNLNRHLKTHLRGS | |
| CMV:ZFP | GCTGGGG | SRPGERPFQCRICMRNFSQSAHLKRHLRTHTGEKPFQCRICMRNF | 2023 |
| 691- | AGAGCTG | SESGHLRRHLKTHTGGGGSQKPFQCRICMRNFSSFQSYLEHLRTH | |
| OFF(mil) | GCGGA | TGEKPFQCRICMRNFSSRRDNLLHLKTHTGSQKPFQCRICMRNFS | |
| | | KKDHLHRHTRTHTGEKPFQCRICMRNFSLSQTLNRHLRTHLRGS | |
| CMV:ZFP | GGGGAGA | SRPGERPFQCRICMRNFSRSRNLLLHTRTHTGEKPFQCRICMRNFS | 2024 |
| 700- | GCTGGCG | QSAHLKRHLRTHTGSQKPFQCRICMRNFSSPSKLARHTRTHTGEK | |
| OFF(mil) | GATGC | PFQCRICMRNFSVKETLTRHLRTHTGGGGSQKPFQCRICMRNFSR | |
| | | QANLVRHTRTHTGEKPFQCRICMRNFSRQEHLVRHLRTHLRGS | |
| CMV:ZFP | GAACAGA | SRPGERPFQCRICMRNFSRQANLVRHTRTHTGEKPFQCRICMRNF | 2025 |
| 708- | TGGTGGG | SQQTNLTRHLRTHTGGGGSQKPFQCRICMRNFSRGTVLRRHTRT | |
| OFF(mil) | AAGAG | HTGEKPFQCRICMRNFSRNDALAVHLRTHTGSQKPFQCRICMRNF | |
| | | SQRRYLVEHTRTHTGEKPFQCRICMRNFSQQTNLARHLRTHLRGS | |
| CMV:ZFP | GTTGGCT | SRPGERPFQCRICMRNFSRNFILQRHTRTHTGEKPFQCRICMRNFS | 2026 |
| 719- | GGGAAGG | EAHHLSRHLRTHTGGGGSQKPFQCRICMRNFSQAVNLARHLRTH | |
| OFF(mil) | GTGTG | TGEKPFQCRICMRNFSRSDHLSLHLKTHTGSQKPFQCRICMRNFS | |
| | | APSKLKRHTRTHTGEKPFQCRICMRNFSTNSSLTRHLRTHLRGS | |
| CMV:ZFP | GATGGTT | SRPGERPFQCRICMRNFSQSSSLVRHLRTHTGEKPFQCRICMRNFS | 2027 |
| 728- | AAAGGGG | RKERLATHLKTHTGSQKPFQCRICMRNFSKGDHLRRHTRTHTGE | |
| OFF(mil) | TGGTA | KPFQCRICMRNFSQRCNLLTHLRTHTGGGGSQKPFQCRICMRNFS | |
| | | TGQKLIVHTRTHTGEKPFQCRICMRNFSVVHNLKRHLRTHLRGS | |
| CMV:ZFP | GCAGCCT | SRPGERPFQCRICMRNFSRGITLTRHTRTHTGEKPFQCRICMRNFS | 2028 |
| 737- | TAAGTTG | VRETLVRHLRTHTGSQKPFQCRICMRNFSHKSSLTRHLRTHTGEK | |
| OFF(mil) | CTACG | PFQCRICMRNFSQSGNLKGHLKTHTGGGGSQKPFQCRICMRNFSD | |
| | | SSVLRRHLRTHTGEKPFQCRICMRNFSQGGTLRRHLKTHLRGS | |
| CMV:ZFP | GGGGGAG | SRPGERPFQCRICMRNFSQQQALVRHTRTHTGEKPFQCRICMRNF | 2029 |
| 746- | AAGTCAG | SRQDNLGRHLRTHTGGGGSQKPFQCRICMRNFSDPSSLKRHLRTH | |
| OFF(mil) | AGGTA | TGEKPFQCRICMRNFSQHPNLTRHLKTHTGSQKPFQCRICMRNFS | |
| | | QSAHLKRHLRTHTGEKPFQCRICMRNFSRDDKLAGHLKTHLRGS | |
| CMV:ZFP | GCTGAGG | SRPGERPFQCRICMRNFSRREHLMRHLRTHTGEKPFQCRICMRNF | 2030 |
| 665- | GCACGAG | SQNSHLRRHLKTHTGGGGSQKPFQCRICMRNFSKKIDLVRHTRTH | |
| OFF(mil) | GAGGG | TGEKPFQCRICMRNFSLKGHLTRHLRTHTGSQKPFQCRICMRNFS | |
| | | RRDNLGRHLRTHTGEKPFQCRICMRNFSVSNTLTRHLKTHLRGS | |
| CMV:ZFP | GGAGCTG | SRPGERPFQCRICMRNFSRKEHLVGHLRTHTGEKPFQCRICMRNF | 2031 |
| 674- | CTGCCTG | SEKSHLTRHLKTHTGGGGSQKPFQCRICMRNFSDPSTLRRHTRTH | |
| OFF(mil) | GCTGG | TGEKPFQCRICMRNFSLRDSLKRHLRTHTGSQKPFQCRICMRNFS | |
| | | QRSDLTRHLRTHTGEKPFQCRICMRNFSQSAHLGRHLKTHLRGS | |
| CMV:ZFP | GCAGCTG | SRPGERPFQCRICMRNFSAPSKLLRHTRTHTGEKPFQCRICMRNFS | 2032 |
| 684- | GGGAGAG | LRDSLKRHLRTHTGGGGSQKPFQCRICMRNFSRQANLTRHTRTH | |
| OFF(mil) | CTGGC | TGEKPFQCRICMRNFSRQEHLVRHLRTHTGSQKPFQCRICMRNFS | |
| | | QRSDLTRHLRTHTGEKPFQCRICMRNFSQGGTLRRHLKTHLRGS | |
| CMV:ZFP | GCTGGGG | SRPGERPFQCRICMRNFSQSAHLKRHLRTHTGEKPFQCRICMRNF | 2033 |
| 692- | AGAGCTG | SESGHLKRHLKTHTGGGGSQKPFQCRICMRNFSFFSYLKKHLRTH | |
| OFF(mil) | GCGGA | TGEKPFQCRICMRNFSRRDNLLRHLKTHTGSQKPFQCRICMRNFS | |
| | | RREHLVRHLRTHTGEKPFQCRICMRNFSVKNTLTRHLKTHLRGS | |
| CMV:ZFP | GGGGAGA | SRPGERPFQCRICMRNFSRTRNLVMHTRTHTGEKPFQCRICMRNF | 2034 |
| 701- | GCTGGCG | SQSAHLKRHLRTHTGSQKPFQCRICMRNFSAPSKLLRHTRTHTGE | |
| OFF(mil) | GATGC | KPFQCRICMRNFSLRDSLKRHLRTHTGGGGSQKPFQCRICMRNFS | |
| | | RNTNLTRHTRTHTGEKPFQCRICMRNFSRREHLVRHLRTHLRGS | |
| CMV:ZFP | GAACAGA | SRPGERPFQCRICMRNFSRQANLVRHTRTHTGEKPFQCRICMRNF | 2035 |
| 710- | TGGTGGG | SQAGNLSRHLRTHTGGGGSQKPFQCRICMRNFSRNFILQRHTRTH | |
| OFF(mil) | AAGAG | TGEKPFQCRICMRNFSRNDVLQSHLRTHTGSQKPFQCRICMRNFS | |
| | | TTFNLRVHTRTHTGEKPFQCRICMRNFSQTQNLTRHLRTHLRGS | |
| CMV:ZFP | TAGGAAC | SRPGERPFQCRICMRNFSTNSMLARHTRTHTGEKPFQCRICMRNF | 2036 |
| 720- | TGGAATT | SQNEHLKVHLRTHTGGGGSQKPFQCRICMRNFSQRSNLARHTRT | |
| OFF(mil) | GAGCT | HTGEKPFQCRICMRNFSRKESLDVHLRTHTGSQKPFQCRICMRNF | |
| | | SQQTNLTRHLRTHTGEKPFQCRICMRNFSRIDHLAGHLKTHLRGS | |
| CMV:ZFP | GATGGTT | SRPGERPFQCRICMRNFSQRSSLVRHLRTHTGEKPFQCRICMRNFS | 2037 |
| 729- | AAAGGGG | RTVALNRHLKTHTGSQKPFQCRICMRNFSKGDHLRRHTRTHTGE | |
| OFF(mil) | TGGTA | KPFQCRICMRNFSQRCNLLTHLRTHTGGGGSQKPFQCRICMRNFS | |
| | | TGQKLIVHTRTHTGEKPFQCRICMRNFSVVHNLKRHLRTHLRGS | |
| CMV:ZFP | GCTACGG | SRPGERPFQCRICMRNFSRSTHLRVHTRTHTGEKPFQCRICMRNFS | 2038 |
| 738- | GAAAGTG | HKSSLTRHLRTHTGGGGSQKPFQCRICMRNFSRQDNLHTHLRTH | |
| OFF(mil) | TTGGA | TGEKPFQCRICMRNFSQRVHLRRHLKTHTGSQKPFQCRICMRNFS | |
| | | RNITLVRHTRTHTGEKPFQCRICMRNFSVKETLVRHLRTHLRGS | |
| CMV:ZFP | AGGGCAC | SRPGERPFQCRICMRNFSRSESLTIHLRTHTGEKPFQCRICMRNFSR | 2039 |
| 667- | GAGGAGG | QGHLKRHLKTHTGSQKPFQCRICMRNFSRPENLNRHLRTHTGEK | |
| OFF(mil) | GGCTG | PFQCRICMRNFSRRDNLNRHLKTHTGGGGSQKPFQCRICMRNFSQ | |
| | | GGTLSRHLRTHTGEKPFQCRICMRNFSRHYRLSTHLKTHLRGS | |
| CMV:ZFP | TGGGGAT | SRPGERPFQCRICMRNFSVPHNLQRHLRTHTGEKPFQCRICMRNF | 2040 |
| 676- | GGGGGTG | SRVDNLGRHLKTHTGGGGSQKPFQCRICMRNFSRVDHLHRHLRT | |
| OFF(mil) | AGGAT | HTGEKPFQCRICMRNFSRSDHLSLHLKTHTGSQKPFQCRICMRNF | |
| | | SQSAHLKRHLRTHTGEKPFQCRICMRNFSRSDHLSLHLKTHLRGS | |
| CMV:ZFP | GCAGCTG | SRPGERPFQCRICMRNFSSPSKLARHTRTHTGEKPFQCRICMRNFS | 2041 |
| 685- | GGGAGAG | QRSDLTRHLRTHTGGGGSQKPFQCRICMRNFSKLTNLARHTRTH | |
| OFF(mil) | CTGGC | TGEKPFQCRICMRNFSRREHLVRHLRTHTGSQKPFQCRICMRNFS | |
| | | QRSDLTRHLRTHTGEKPFQCRICMRNFSQGGTLRRHLKTHLRGS | |
| CMV:ZFP | GCTGGGG | SRPGERPFQCRICMRNFSQSAHLKRHLRTHTGEKPFQCRICMRNF | 2042 |
| 693- | AGAGCTG | SEKSHLKRHLKTHTGGGGSQKPFQCRICMRNFSFHSYLQKHLRT | |
| OFF(mil) | GCGGA | HTGEKPFQCRICMRNFSRRDNLLRHLKTHTGSQKPFQCRICMRNF | |
| | | SRREHLVRHLRTHTGEKPFQCRICMRNFSVKNTLTRHLKTHLRGS | |
| CMV:ZFP | GACGGGG | SRPGERPFQCRICMRNFSRGTVLRRHTRTHTGEKPFQCRICMRNF | 2043 |
| 702- | GAACAGA | SRNDALAVHLRTHTGSQKPFQCRICMRNFSQRRYLVEHTRTHTG | |
| OFF(mil) | TGGTG | EKPFQCRICMRNFSQQTNLARHLRTHTGGGGSQKPFQCRICMRNF | |
| | | SRREHLVRHLRTHTGEKPFQCRICMRNFSDGGNLGRHLKTHLRG | |
| | | S | |
| CMV:ZFP | GAACAGA | SRPGERPFQCRICMRNFSRQANLVRHTRTHTGEKPFQCRICMRNF | 2044 |
| 711- | TGGTGGG | SQAGNLSRHLRTHTGGGGSQKPFQCRICMRNFSRNFILQRHTRTH | |
| OFF(mil) | AAGAG | TGEKPFQCRICMRNFSRNDVLQSHLRTHTGSQKPFQCRICMRNFS | |
| | | QRRYLVEHTRTHTGEKPFQCRICMRNFSQQTNLARHLRTHLRGS | |
| CMV:ZFP | TAGGAAC | SRPGERPFQCRICMRNFSHHTSLVRHTRTHTGEKPFQCRICMRNFS | 2045 |
| 721- | TGGAATT | QREHLKVHLRTHTGGGGSQKPFQCRICMRNFSQRGNLARHLRTH | |
| OFF(mil) | GAGCT | TGEKPFQCRICMRNFSRNHSLRMHLKTHTGSQKPFQCRICMRNFS | |
| | | QQTNLTRHLRTHTGEKPFQCRICMRNFSRPESLAPHLKTHLRGS | |
| CMV:ZFP | GATGGTT | SRPGERPFQCRICMRNFSQRSSLVRHLRTHTGEKPFQCRICMRNFS | 2046 |
| 730- | AAAGGGG | RTVALNRHLKTHTGSQKPFQCRICMRNFSKGDHLRRHTRTHTGE | |
| OFF(mil) | TGGTA | KPFQCRICMRNFSQRCNLLTHLRTHTGGGGSQKPFQCRICMRNFS | |
| | | IPNHLARHTRTHTGEKPFQCRICMRNFSVRHNLTRHLRTHLRGS | |
| CMV:ZFP | GCTACGG | SRPGERPFQCRICMRNFSQSAHLKRHLRTHTGEKPFQCRICMRNF | 2047 |
| 739- | GAAAGTG | SHHNSLTRHLKTHTGGGGSQKPFQCRICMRNFSRQDNLHTHLRT | |
| OFF(mil) | TTGGA | HTGEKPFQCRICMRNFSQNSHLRRHLKTHTGSQKPFQCRICMRNF | |
| | | SRNITLVRHTRTHTGEKPFQCRICMRNFSVKETLVRHLRTHLRGS | |
| CMV:ZFP | AGGGCAC | SRPGERPFQCRICMRNFSRSESLTIHLRTHTGEKPFQCRICMRNFSR | 2048 |
| 668- | GAGGAGG | QGHLKRHLKTHTGSQKPFQCRICMRNFSRPENLNRHLRTHTGEK | |
| OFF(mil) | GGCTG | PFQCRICMRNFSRRDNLNRHLKTHTGGGGSQKPFQCRICMRNFSQ | |
| | | SGTLKRHLRTHTGEKPFQCRICMRNFSRPYVLSYHLKTHLRGS | |
| CMV:ZFP | TGGGGAT | SRPGERPFQCRICMRNFSVNHNLRRHLRTHTGEKPFQCRICMRNF | 2049 |
| 677- | GGGGGTG | SRQDNLQRHLKTHTGGGGSQKPFQCRICMRNFSRVDHLHRHLRT | |
| OFF(mil) | AGGAT | HTGEKPFQCRICMRNFSRSDHLSLHLKTHTGSQKPFQCRICMRNF | |
| | | SQSAHLKRHLRTHTGEKPFQCRICMRNFSRSDHLSLHLKTHLRGS | |
| CMV:ZFP | GCAGCTG | SRPGERPFQCRICMRNFSAPSKLLRHTRTHTGEKPFQCRICMRNFS | 2050 |
| 686- | GGGAGAG | LRDSLKRHLRTHTGGGGSQKPFQCRICMRNFSKLTNLARHTRTH | |
| OFF(mil) | CTGGC | TGEKPFQCRICMRNFSRREHLVRHLRTHTGSQKPFQCRICMRNFS | |
| | | TKQVLDRHTRTHTGEKPFQCRICMRNFSQSTTLKRHLRTHLRGS | |
| CMV:ZFP | GCTGGGG | SRPGERPFQCRICMRNFSQSAHLKRHLRTHTGEKPFQCRICMRNF | 2051 |
| 694- | AGAGCTG | SESGHLKRHLKTHTGGGGSQKPFQCRICMRNFSSFQSYLEHLRTH | |
| OFF(mil) | GCGGA | TGEKPFQCRICMRNFSSRRDNLLHLKTHTGSQKPFQCRICMRNFS | |
| | | KKDHLHRHTRTHTGEKPFQCRICMRNFSLSQTLNRHLRTHLRGS | |
| CMV:ZFP | GACGGGG | SRPGERPFQCRICMRNFSRNFILQRHTRTHTGEKPFQCRICMRNFS | 2052 |
| 703- | GAACAGA | RNDVLQSHLRTHTGSQKPFQCRICMRNFSQRRYLVEHTRTHTGE | |
| OFF(mil) | TGGTG | KPFQCRICMRNFSQQTNLARHLRTHTGGGGSQKPFQCRICMRNFS | |
| | | RREHLVRHLRTHTGEKPFQCRICMRNFSDGGNLGRHLKTHLRGS | |
| CMV:ZFP | GTTGGCT | SRPGERPFQCRICMRNFSRREVLENHLRTHTGEKPFQCRICMRNFS | 2053 |
| 714- | GGGAAGG | IKHHLGRHLKTHTGGGGSQKPFQCRICMRNFSQAVNLARHLRTH | |
| OFF(mil) | GTGTG | TGEKPFQCRICMRNFSRSDHLSLHLKTHTGSQKPFQCRICMRNFS | |
| | | APSKLKRHTRTHTGEKPFQCRICMRNFSTNSSLTRHLRTHLRGS | |
| CMV:ZFP | TAGGAAC | SRPGERPFQCRICMRNFSTNSMLARHTRTHTGEKPFQCRICMRNF | 2054 |
| 722- | TGGAATT | SQNEHLKVHLRTHTGGGGSQKPFQCRICMRNFSQRSNLARHTRT | |
| OFF(mil) | GAGCT | HTGEKPFQCRICMRNFSRKESLDVHLRTHTGSQKPFQCRICMRNF | |
| | | SQQTNLTRHLRTHTGEKPFQCRICMRNFSRPESLAPHLKTHLRGS | |
| CMV:ZFP | GGTGTGG | SRPGERPFQCRICMRNFSDSPTLRRHTRTHTGEKPFQCRICMRNFS | 2055 |
| 732- | TTGGGTG | QSTTLKRHLRTHTGGGGSQKPFQCRICMRNFSRREHLVRHLRTHT | |
| OFF(mil) | CAGCC | GEKPFQCRICMRNFSVNSSLGRHLKTHTGSQKPFQCRICMRNFSR | |
| | | NFILQRHTRTHTGEKPFQCRICMRNFSEAHHLSRHLRTHLRGS | |
| CMV:ZFP | GCTACGG | SRPGERPFQCRICMRNFSSRSTHLRHTRTHTGEKPFQCRICMRNFS | 2056 |
| 740- | GAAAGTG | SHKSSLTHLRTHTGGGGSQKPFQCRICMRNFSRQDNLHTHLRTHT | |
| OFF(mil) | TTGGA | GEKPFQCRICMRNFSQNSHLRRHLKTHTGSQKPFQCRICMRNFSR | |
| | | NITLVRHTRTHTGEKPFQCRICMRNFSVKETLVRHLRTHLRGS | |
| CMV:ZFP | GGAGGAC | SRPGERPFQCRICMRNFSYKESLVRHLRTHTGEKPFQCRICMRNFS | 2057 |
| 747- | CCAGCAG | QGGHLARHLKTHTGSQKPFQCRICMRNFSQSGTLKRHLRTHTGE | |
| OFF(mil) | GAATC | KPFQCRICMRNFSKQWYLQLHLKTHTGGGGSQKPFQCRICMRNF | |
| | | SQSAHLKRHLRTHTGEKPFQCRICMRNFSQNSHLRRHLKTHLRGS | |
| CMV:ZFP | GAGAGCC | SRPGERPFQCRICMRNFSDPANLRRHTRTHTGEKPFQCRICMRNF | 2058 |
| 757- | TCTGCTGG | SRQEHLVRHLRTHTGSQKPFQCRICMRNFSLRDSLKRHLRTHTGE | |
| OFF(mil) | GGAC | KPFQCRICMRNFSQRNALHGHLKTHTGGGGSQKPFQCRICMRNF | |
| | | SFFSYLKKHLRTHTGEKPFQCRICMRNFSRRDNLLRHLKTHLRGS | |
| CMV:ZFP | ACAGAAA | SRPGERPFQCRICMRNFSKKDHLHRHTRTHTGEKPFQCRICMRNF | 2059 |
| 765- | GTCTGTTG | SRREHLTIHLRTHTGSQKPFQCRICMRNFSRKQHLVLHTRTHTGE | |
| OFF(mil) | GGGG | KPFQCRICMRNFSDHSSLKRHLRTHTGGGGSQKPFQCRICMRNFS | |
| | | QRGNLTRHLRTHTGEKPFQCRICMRNFSQSNSLKYHLKTHLRGS | |
| CMV:ZFP | GTGGACT | SRPGERPFQCRICMRNFSQQTNLTRHLRTHTGEKPFQCRICMRNF | 2060 |
| 774- | GAATTTG | SIRHHLKRHLKTHTGSQKPFQCRICMRNFSLRSILKAHTRTHTGEK | |
| OFF(mil) | GTGAA | PFQCRICMRNFSQAGNLSRHLRTHTGGGGSQKPFQCRICMRNFSD | |
| | | RSNLTRHLRTHTGEKPFQCRICMRNFSRPDALPRHLKTHLRGS | |
| CMV:ZFP | TAGGACC | SRPGERPFQCRICMRNFSRREVLENHLRTHTGEKPFQCRICMRNFS | 2061 |
| 782- | CAGCAGG | DTGHLKRHLKTHTGSQKPFQCRICMRNFSKKFNLLQHTRTHTGE | |
| OFF(mil) | GCGTG | KPFQCRICMRNFSRRDNLKSHLRTHTGGGGSQKPFQCRICMRNFS | |
| | | DRGNLTRHLRTHTGEKPFQCRICMRNFSRKTGLLIHLKTHLRGS | |
| CMV:ZFP | GACAGGT | SRPGERPFQCRICMRNFSKKFNLLQHTRTHTGEKPFQCRICMRNF | 2062 |
| 790- | AGGACCC | SRQDNLNSHLRTHTGGGGSQKPFQCRICMRNFSDPSNLQRHLRT | |
| OFF(mil) | AGCAG | HTGEKPFQCRICMRNFSRRDNLPKHLKTHTGSQKPFQCRICMRNF | |
| | | SRRAHLLNHTRTHTGEKPFQCRICMRNFSDRGNLTRHLRTHLRGS | |
| CMV:ZFP | GGGGAGG | SRPGERPFQCRICMRNFSDRSVLKRHLRTHTGEKPFQCRICMRNF | 2063 |
| 798- | GCAGCAT | SDKRSLPVHLKTHTGSQKPFQCRICMRNFSQRGTLNRHTRTHTGE | |
| OFF(mil) | CCGCC | KPFQCRICMRNFSQSTTLKRHLRTHTGGGGSQKPFQCRICMRNFS | |
| | | RDDNLQRHLRTHTGEKPFQCRICMRNFSRMEHLPRHLKTHLRGS | |
| CMV:ZFP | GGAGGAC | SRPGERPFQCRICMRNFSYKESLVRHLRTHTGEKPFQCRICMRNFS | 2064 |
| 748- | CCAGCAG | QGGHLARHLKTHTGSQKPFQCRICMRNFSQSGTLKRHLRTHTGE | |
| OFF(mil) | GAATC | KPFQCRICMRNFSKQWYLQLHLKTHTGGGGSQKPFQCRICMRNF | |
| | | SQSAHLKRHLRTHTGEKPFQCRICMRNFSQTTHLSRHLKTHLRGS | |
| CMV:ZFP | GAGAGCC | SRPGERPFQCRICMRNFSEEANLRRHTRTHTGEKPFQCRICMRNFS | 2065 |
| 758- | TCTGCTGG | RREHLVRHLRTHTGSQKPFQCRICMRNFSLRDSLKRHLRTHTGEK | |
| OFF(mil) | GGAC | PFQCRICMRNFSQRNALHGHLKTHTGGGGSQKPFQCRICMRNFSF | |
| | | FSYLKKHLRTHTGEKPFQCRICMRNFSRRDNLLRHLKTHLRGS | |
| CMV:ZFP | ACAGAAA | SRPGERPFQCRICMRNFSRGHHLDRHTRTHTGEKPFQCRICMRNF | 2066 |
| 766- | GTCTGTTG | SRNEHLVLHLRTHTGSQKPFQCRICMRNFSRKQHLVLHTRTHTGE | |
| OFF(mil) | GGGG | KPFQCRICMRNFSDHSSLKRHLRTHTGGGGSQKPFQCRICMRNFS | |
| | | QRGNLTRHLRTHTGEKPFQCRICMRNFSQSNSLKYHLKTHLRGS | |
| CMV:ZFP | GTGGACT | SRPGERPFQCRICMRNFSQQTNLTRHLRTHTGEKPFQCRICMRNF | 2067 |
| 775- | GAATTTG | SHGHRLKTHLKTHTGSQKPFQCRICMRNFSLRSILKAHTRTHTGE | |
| OFF(mil) | GTGAA | KPFQCRICMRNFSQAGNLSRHLRTHTGGGGSQKPFQCRICMRNFS | |
| | | DRSNLTRHLRTHTGEKPFQCRICMRNFSRPDALPRHLKTHLRGS | |
| CMV:ZFP | TAGGACC | SRPGERPFQCRICMRNFSTTYHLIRHTRTHTGEKPFQCRICMRNFS | 2068 |
| 783- | CAGCAGG | ENSKLKRHLRTHTGSQKPFQCRICMRNFSKKFNLLQHTRTHTGEK | |
| OFF(mil) | GCGTG | PFQCRICMRNFSRRDNLKSHLRTHTGGGGSQKPFQCRICMRNFSD | |
| | | GSNLRRHLRTHTGEKPFQCRICMRNFSRIDNLDGHLKTHLRGS | |
| CMV:ZFP | GACAGGT | SRPGERPFQCRICMRNFSKRYNLYQHTRTHTGEKPFQCRICMRNF | 2069 |
| 791- | AGGACCC | SRQDNLNTHLRTHTGGGGSQKPFQCRICMRNFSDGSNLRRHLRT | |
| OFF(mil) | AGCAG | HTGEKPFQCRICMRNFSRIDNLDGHLKTHTGSQKPFQCRICMRNF | |
| | | SRKDHLKTHLRTHTGEKPFQCRICMRNFSEGGNLMRHLKTHLRG | |
| | | S | |
| CMV:ZFP | GGGGAGG | SRPGERPFQCRICMRNFSDRSVLKRHLRTHTGEKPFQCRICMRNF | 2070 |
| 799- | GCAGCAT | SDKRSLPVHLKTHTGSQKPFQCRICMRNFSQRGTLNRHTRTHTGE | |
| OFF(mil) | CCGCC | KPFQCRICMRNFSQSTTLKRHLRTHTGGGGSQKPFQCRICMRNFS | |
| | | REDNLDRHLRTHTGEKPFQCRICMRNFSRRHGLGRHLKTHLRGS | |
| CMV:ZFP | GGAGGAC | SRPGERPFQCRICMRNFSYKESLVRHLRTHTGEKPFQCRICMRNFS | 2071 |
| 749- | CCAGCAG | QGGHLARHLKTHTGSQKPFQCRICMRNFSQSGTLKRHLRTHTGE | |
| OFF(mil) | GAATC | KPFQCRICMRNFSKQWYLQLHLKTHTGGGGSQKPFQCRICMRNF | |
| | | SQSAHLKRHLRTHTGEKPFQCRICMRNFSQMSHLKRHLKTHLRG | |
| | | S | |
| CMV:ZFP | GAGAGCC | SRPGERPFQCRICMRNFSEEANLRRHTRTHTGEKPFQCRICMRNFS | 2072 |
| 759- | TCTGCTGG | RGEHLTRHLRTHTGSQKPFQCRICMRNFSVKHSLQRHLRTHTGE | |
| OFF(mil) | GGAC | KPFQCRICMRNFSQRNTLKGHLKTHTGGGGSQKPFQCRICMRNFS | |
| | | SFQSYLEHLRTHTGEKPFQCRICMRNFSSRRDNLLHLKTHLRGS | |
| CMV:ZFP | ACAGAAA | SRPGERPFQCRICMRNFSRKHHLGRHTRTHTGEKPFQCRICMRNF | 2073 |
| 767- | GTCTGTTG | SRREHLTIHLRTHTGSQKPFQCRICMRNFSRKQHLALHTRTHTGE | |
| OFF(mil) | GGGG | KPFQCRICMRNFSDHSSLKRHLRTHTGGGGSQKPFQCRICMRNFS | |
| | | QQTNLTRHLRTHTGEKPFQCRICMRNFSQGGILYRHLKTHLRGS | |
| CMV:ZFP | GTGGACT | SRPGERPFQCRICMRNFSQQTNLTRHLRTHTGEKPFQCRICMRNF | 2074 |
| 776- | GAATTTG | SIRHHLKRHLKTHTGSQKPFQCRICMRNFSLRSILKAHTRTHTGEK | |
| OFF(mil) | GTGAA | PFQCRICMRNFSQAGNLSRHLRTHTGGGGSQKPFQCRICMRNFSD | |
| | | ASNLARHLRTHTGEKPFQCRICMRNFSRHTSLTRHLKTHLRGS | |
| CMV:ZFP | TAGGACC | SRPGERPFQCRICMRNFSRREVLENHLRTHTGEKPFQCRICMRNFS | 2075 |
| 784- | CAGCAGG | DTGHLKRHLKTHTGSQKPFQCRICMRNFSKKENLLQHTRTHTGE | |
| OFF(mil) | GCGTG | KPFQCRICMRNFSRRDNLKSHLRTHTGGGGSQKPFQCRICMRNFS | |
| | | DGSNLRRHLRTHTGEKPFQCRICMRNFSRIDNLDGHLKTHLRGS | |
| CMV:ZFP | TGGGTAT | SRPGERPFQCRICMRNFSSKPNLKMHTRTHTGEKPFQCRICMRNF | 2076 |
| 792- | GGAGTGG | SRGDNLGRHLRTHTGSQKPFQCRICMRNFSRREVLENHLRTHTGE | |
| OFF(mil) | AGTCT | KPFQCRICMRNFSQSAHLGRHLKTHTGGGGSQKPFQCRICMRNFS | |
| | | QRSSLVRHLRTHTGEKPFQCRICMRNFSRTDHLGLHLKTHLRGS | |
| CMV:ZFP | GGGGAGG | SRPGERPFQCRICMRNFSDRSVLKRHLRTHTGEKPFQCRICMRNF | 2077 |
| 800- | GCAGCAT | SDKRSLAPHLKTHTGSQKPFQCRICMRNFSQAETLKRHLRTHTGE | |
| OFF(mil) | CCGCC | KPFQCRICMRNFSQGGTLRRHLKTHTGGGGSQKPFQCRICMRNFS | |
| | | RDDNLQRHLRTHTGEKPFQCRICMRNFSRMEHLPRHLKTHLRGS | |
| CMV:ZFP | GTGGTAT | SRPGERPFQCRICMRNFSLKQHLVVHLRTHTGEKPFQCRICMRNF | 2078 |
| 751- | CAAGGAG | SQAGHLTRHLKTHTGSQKPFQCRICMRNFSDNAHLARHTRTHTG | |
| OFF(mil) | GAAGT | EKPFQCRICMRNFSQKCNLKAHLRTHTGGGGSQKPFQCRICMRN | |
| | | FSQRSSLVRHLRTHTGEKPFQCRICMRNFSRTVALNRHLKTHLRG | |
| | | S | |
| CMV:ZFP | GAGAGCC | SRPGERPFQCRICMRNFSEEANLRRHTRTHTGEKPFQCRICMRNFS | 2079 |
| 760- | TCTGCTGG | RREHLVRHLRTHTGSQKPFQCRICMRNFSQRSDLTRHLRTHTGEK | |
| OFF(mil) | GGAC | PFQCRICMRNFSQRNTLKGHLKTHTGGGGSQKPFQCRICMRNFSF | |
| | | FSYLKKHLRTHTGEKPFQCRICMRNFSRRDNLLRHLKTHLRGS | |
| CMV:ZFP | GAATTTG | SRPGERPFQCRICMRNFSQRGNLTRHLRTHTGEKPFQCRICMRNF | 2080 |
| 768- | GTGAAGA | SQSNSLKYHLKTHTGGGGSQKPFQCRICMRNFSQQTNLTRHLRT | |
| OFF(mil) | CAGAA | HTGEKPFQCRICMRNFSHGHRLKTHLKTHTGSQKPFQCRICMRNF | |
| | | STRTELNVHTRTHTGEKPFQCRICMRNFSQAGNLRRHLRTHLRGS | |
| CMV:ZFP | GCAACGC | SRPESLAPHLKTHTGSQKPFQCRICMRNFSRTSSLKRHTRTHTGEK | 2081 |
| 777- | ATTGTGTA | SRPGERPFQCRICMRNFSQQTNLTRHLRTHTGEKPFQCRICMRNF | |
| OFF(mil) | GGAA | PFQCRICMRNFSHRESLTVHLRTHTGGGGSQKPFQCRICMRNFSR | |
| | | PDTLVVHLRTHTGEKPFQCRICMRNFSQGGTLRRHLKTHLRGS | |
| CMV:ZFP | TAGGACC | SRPGERPFQCRICMRNFSRREVLENHLRTHTGEKPFQCRICMRNFS | 2082 |
| 785- | CAGCAGG | DTGHLKRHLKTHTGSQKPFQCRICMRNFSKKFNLLQHTRTHTGE | |
| OFF(mil) | GCGTG | KPFQCRICMRNFSRKDYLISHLRTHTGGGGSQKPFQCRICMRNFS | |
| | | DRGNLTRHLRTHTGEKPFQCRICMRNFSRKTGLLIHLKTHLRGS | |
| CMV:ZFP | TGGGTAT | SRPGERPFQCRICMRNFSSKPNLKMHTRTHTGEKPFQCRICMRNF | 2083 |
| 793- | GGAGTGG | SRGDNLGRHLRTHTGSQKPFQCRICMRNFSRNFILQRHTRTHTGE | |
| OFF(mil) | AGTCT | KPFQCRICMRNFSQSAHLKRHLRTHTGGGGSQKPFQCRICMRNFS | |
| | | QRSSLVRHLRTHTGEKPFQCRICMRNFSRSDHLSLHLKTHLRGS | |
| CMV:ZFP | GAGGCTG | SRPGERPFQCRICMRNFSRADNLGRHLRTHTGEKPFQCRICMRNF | 2084 |
| 804- | TGTGCTTC | SARNTLKGHLKTHTGGGGSQKPFQCRICMRNFSRRRNLTLHTRT | |
| OFF(mil) | TGAG | HTGEKPFQCRICMRNFSRPHVLANHLRTHTGSQKPFQCRICMRNF | |
| | | SVKETLVRHLRTHTGEKPFQCRICMRNFSRQDNLQRHLKTHLRG | |
| | | S | |
| CMV:ZFP | GTGGTAT | SRPGERPFQCRICMRNFSQKHHLAVHLRTHTGEKPFQCRICMRNF | 2085 |
| 752- | CAAGGAG | SQGGHLKRHLKTHTGSQKPFQCRICMRNFSDNAHLARHTRTHTG | |
| OFF(mil) | GAAGT | EKPFQCRICMRNFSQNCNLKSHLRTHTGGGGSQKPFQCRICMRNF | |
| | | SQRSSLVRHLRTHTGEKPFQCRICMRNFSRKDALHVHLKTHLRGS | |
| CMV:ZFP | GAGAGCC | SRPGERPFQCRICMRNFSEEANLRRHTRTHTGEKPFQCRICMRNFS | 2086 |
| 761- | TCTGCTGG | RGEHLTRHLRTHTGSQKPFQCRICMRNFSQRSDLTRHLRTHTGEK | |
| OFF(mil) | GGAC | PFQCRICMRNFSQRNTLKGHLKTHTGGGGSQKPFQCRICMRNFSF | |
| | | HSYLQKHLRTHTGEKPFQCRICMRNFSRRDNLLRHLKTHLRGS | |
| CMV:ZFP | GAATTTG | SRPGERPFQCRICMRNFSQQTNLTRHLRTHTGEKPFQCRICMRNF | 2087 |
| 769- | GTGAAGA | SQGGILYRHLKTHTGGGGSQKPFQCRICMRNFSQQTNLTRHLRTH | |
| OFF(mil) | CAGAA | TGEKPFQCRICMRNFSHGHRLKTHLKTHTGSQKPFQCRICMRNFS | |
| | | TRTELNVHTRTHTGEKPFQCRICMRNFSQAGNLRRHLRTHLRGS | |
| CMV:ZFP | GCAACGC | SRPGERPFQCRICMRNFSQQTNLTRHLRTHTGEKPFQCRICMRNF | 2088 |
| 778- | ATTGTGTA | SRPESLAPHLKTHTGSQKPFQCRICMRNFSRTSSLKRHTRTHTGEK | |
| OFF(mil) | GGAA | PFQCRICMRNFSHRESLTVHLRTHTGGGGSQKPFQCRICMRNFSR | |
| | | SQTLKQHTRTHTGEKPFQCRICMRNFSQSTTLKRHLRTHLRGS | |
| CMV:ZFP | GACAGGT | SRPGERPFQCRICMRNFSKKFNLLQHTRTHTGEKPFQCRICMRNF | 2089 |
| 786- | AGGACCC | SRQDNLNSHLRTHTGGGGSQKPFQCRICMRNFSDRGNLTRHLRT | |
| OFF(mil) | AGCAG | HTGEKPFQCRICMRNFSRKTGLLIHLKTHTGSQKPFQCRICMRNFS | |
| | | RRAHLLNHTRTHTGEKPFQCRICMRNFSDRGNLTRHLRTHLRGS | |
| CMV:ZFP | TGGGTAT | SRPGERPFQCRICMRNFSSKPNLKMHTRTHTGEKPFQCRICMRNF | 2090 |
| 794- | GGAGTGG | SRGDNLGRHLRTHTGSQKPFQCRICMRNFSRREVLENHLRTHTGE | |
| OFF(mil) | AGTCT | KPFQCRICMRNFSQKPHLSRHLKTHTGGGGSQKPFQCRICMRNFS | |
| | | QRSSLVRHLRTHTGEKPFQCRICMRNFSRTDHLGLHLKTHLRGS | |
| CMV:ZFP | GAGGCTG | SRPGERPFQCRICMRNFSRQDNLGRHLRTHTGEKPFQCRICMRNF | 2091 |
| 806- | TGTGCTTC | SQRNALAGHLKTHTGGGGSQKPFQCRICMRNFSRRRNLTLHTRT | |
| OFF(mil) | TGAG | HTGEKPFQCRICMRNFSRVEVLTNHLRTHTGSQKPFQCRICMRNF | |
| | | SVKETLVRHLRTHTGEKPFQCRICMRNFSRQDNLQRHLKTHLRG | |
| | | S | |
| CMV:ZFP | GTAGTCA | SRPGERPFQCRICMRNFSRNTHLARHTRTHTGEKPFQCRICMRNF | 2092 |
| 753- | GATGGTTT | SRADVLKGHLRTHTGSQKPFQCRICMRNFSTTTKLAIHTRTHTGE | |
| OFF(mil) | GGGG | KPFQCRICMRNFSVNHNLTRHLRTHTGGGGSQKPFQCRICMRNFS | |
| | | TGAVLTRHTRTHTGEKPFQCRICMRNFSQRSSLVRHLRTHLRGS | |
| CMV:ZFP | ACAGAAA | SRPGERPFQCRICMRNFSKKDHLHRHTRTHTGEKPFQCRICMRNF | 2093 |
| 762- | GTCTGTTG | SRREHLTIHLRTHTGSQKPFQCRICMRNFSRKQHLALHTRTHTGE | |
| OFF(mil) | GGGG | KPFQCRICMRNFSDHSSLKRHLRTHTGGGGSQKPFQCRICMRNFS | |
| | | QRGNLTRHLRTHTGEKPFQCRICMRNFSQSNSLKYHLKTHLRGS | |
| CMV:ZFP | GTGGACT | SRPGERPFQCRICMRNFSQQTNLTRHLRTHTGEKPFQCRICMRNF | 2094 |
| 771- | GAATTTG | SLTQHLVRHLKTHTGSQKPFQCRICMRNFSTRTELNVHTRTHTGE | |
| OFF(mil) | GTGAA | KPFQCRICMRNFSQAGNLRRHLRTHTGGGGSQKPFQCRICMRNFS | |
| | | LGENLRRHLRTHTGEKPFQCRICMRNFSRGDALARHLKTHLRGS | |
| CMV:ZFP | GCAACGC | SRPGERPFQCRICMRNFSQQTNLTRHLRTHTGEKPFQCRICMRNF | 2095 |
| 779- | ATTGTGTA | SRRDHLSLHLKTHTGSQKPFQCRICMRNFSRTSSLKRHTRTHTGE | |
| OFF(mil) | GGAA | KPFQCRICMRNFSHRESLTVHLRTHTGGGGSQKPFQCRICMRNFS | |
| | | RPDTLVVHLRTHTGEKPFQCRICMRNFSQGGTLRRHLKTHLRGS | |
| CMV:ZFP | GACAGGT | SRPGERPFQCRICMRNFSKRYNLYQHTRTHTGEKPFQCRICMRNF | 2096 |
| 787- | AGGACCC | SRQDNLNTHLRTHTGGGGSQKPFQCRICMRNFSDGSNLRRHLRT | |
| OFF(mil) | AGCAG | HTGEKPFQCRICMRNFSRIDNLDGHLKTHTGSQKPFQCRICMRNF | |
| | | SRRAHLLNHTRTHTGEKPFQCRICMRNFSDRGNLTRHLRTHLRGS | |
| CMV:ZFP | GGGGAGG | SRPGERPFQCRICMRNFSDRSVLKRHLRTHTGEKPFQCRICMRNF | 2097 |
| 795- | GCAGCAT | SDKRSLPVHLKTHTGSQKPFQCRICMRNFSQGGTLKRHLRTHTGE | |
| OFF(mil) | CCGCC | KPFQCRICMRNFSQGGTLRRHLKTHTGGGGSQKPFQCRICMRNFS | |
| | | RDDNLQRHLRTHTGEKPFQCRICMRNFSRMEHLPRHLKTHLRGS | |
| CMV:ZFP | GAGGCTG | SRPGERPFQCRICMRNFSRQDNLGRHLRTHTGEKPFQCRICMRNF | 2098 |
| 807- | TGTGCTTC | SQRNALAGHLKTHTGGGGSQKPFQCRICMRNFSRRRNLTLHTRT | |
| OFF(mil) | TGAG | HTGEKPFQCRICMRNFSRKQVLTNHLRTHTGSQKPFQCRICMRNF | |
| | | SVKETLVRHLRTHTGEKPFQCRICMRNFSRQDNLQRHLKTHLRG | |
| | | S | |
| CMV:ZFP | GTAGTCA | SRPGERPFQCRICMRNFSRNTHLARHTRTHTGEKPFQCRICMRNF | 2099 |
| 755- | GATGGTTT | SRAESLRVHLRTHTGSQKPFQCRICMRNFSTTTKLAIHTRTHTGE | |
| OFF(mil) | GGGG | KPFQCRICMRNFSVNHNLTRHLRTHTGGGGSQKPFQCRICMRNFS | |
| | | DHSSLKRHLRTHTGEKPFQCRICMRNFSQSTSLQRHLKTHLRGS | |
| CMV:ZFP | ACAGAAA | SRPGERPFQCRICMRNFSRKHHLGRHTRTHTGEKPFQCRICMRNF | 2100 |
| 763- | GTCTGTTG | SRREHLTIHLRTHTGSQKPFQCRICMRNFSRKQHLVLHTRTHTGE | |
| OFF(mil) | GGGG | KPFQCRICMRNFSDHSSLKRHLRTHTGGGGSQKPFQCRICMRNFS | |
| | | QRGNLTRHLRTHTGEKPFQCRICMRNFSQSNSLKYHLKTHLRGS | |
| CMV:ZFP | GTGGACT | SRPGERPFQCRICMRNFSQQTNLTRHLRTHTGEKPFQCRICMRNF | 2101 |
| 772- | GAATTTG | SHGHRLKTHLKTHTGSQKPFQCRICMRNFSLRSILKAHTRTHTGE | |
| OFF(mil) | GTGAA | KPFQCRICMRNFSQAGNLSRHLRTHTGGGGSQKPFQCRICMRNFS | |
| | | DASNLARHLRTHTGEKPFQCRICMRNFSRHTSLTRHLKTHLRGS | |
| CMV:ZFP | TAGGACC | SRPGERPFQCRICMRNFSRREVLENHLRTHTGEKPFQCRICMRNFS | 2102 |
| 780- | CAGCAGG | DTGHLKRHLKTHTGSQKPFQCRICMRNFSKKFNLLQHTRTHTGE | |
| OFF(mil) | GCGTG | KPFQCRICMRNFSRQDNLNSHLRTHTGGGGSQKPFQCRICMRNFS | |
| | | DGSNLRRHLRTHTGEKPFQCRICMRNFSRIDNLDGHLKTHLRGS | |
| CMV:ZFP | GACAGGT | SRPGERPFQCRICMRNFSKKFNLLQHTRTHTGEKPFQCRICMRNF | 2103 |
| 788- | AGGACCC | SRQDNLNSHLRTHTGGGGSQKPFQCRICMRNFSDPSNLQRHLRT | |
| OFF(mil) | AGCAG | HTGEKPFQCRICMRNFSRRDNLPKHLKTHTGSQKPFQCRICMRNF | |
| | | SRKDHLKTHLRTHTGEKPFQCRICMRNFSEGGNLMRHLKTHLRG | |
| | | S | |
| CMV:ZFP | GGGGAGG | SRPGERPFQCRICMRNFSDRSVLKRHLRTHTGEKPFQCRICMRNF | 2104 |
| 796- | GCAGCAT | SDKRSLAPHLKTHTGSQKPFQCRICMRNFSQGGTLKRHLRTHTGE | |
| OFF(mil) | CCGCC | KPFQCRICMRNFSQGGTLRRHLKTHTGGGGSQKPFQCRICMRNFS | |
| | | RADNLGRHLRTHTGEKPFQCRICMRNFSRMEHLPRHLKTHLRGS | |
| CMV:ZFP | GAGGCTG | SRPGERPFQCRICMRNFSRQDNLGRHLRTHTGEKPFQCRICMRNF | 2105 |
| 808- | TGTGCTTC | SQRNALAGHLKTHTGGGGSQKPFQCRICMRNFSRRRNLTLHTRT | |
| OFF(mil) | TGAG | HTGEKPFQCRICMRNFSRPHVLANHLRTHTGSQKPFQCRICMRNF | |
| | | SVRETLVRHLRTHTGEKPFQCRICMRNFSRGDNLKRHLKTHLRGS | |
| CMV:ZFP | GAGAGCC | SRPGERPFQCRICMRNFSEEANLRRHTRTHTGEKPFQCRICMRNFS | 2106 |
| 756- | TCTGCTGG | RREHLVRHLRTHTGSQKPFQCRICMRNFSQRSDLTRHLRTHTGEK | |
| OFF(mil) | GGAC | PFQCRICMRNFSQRNTLKGHLKTHTGGGGSQKPFQCRICMRNESS | |
| | | FQSYLEHLRTHTGEKPFQCRICMRNFSSRRDNLLHLKTHLRGS | |
| CMV:ZFP | ACAGAAA | SRPGERPFQCRICMRNFSRGHHLDRHTRTHTGEKPFQCRICMRNF | 2107 |
| 764- | GTCTGTTG | SRNEHLVLHLRTHTGSQKPFQCRICMRNFSRKQHLVLHTRTHTGE | |
| OFF(mil) | GGGG | KPFQCRICMRNFSDHSSLKRHLRTHTGGGGSQKPFQCRICMRNFS | |
| | | QQTNLTRHLRTHTGEKPFQCRICMRNFSQGGILYRHLKTHLRGS | |
| CMV:ZFP | GTGGACT | SRPGERPFQCRICMRNFSQQTNLTRHLRTHTGEKPFQCRICMRNF | 2108 |
| 773- | GAATTTG | SIRHHLKRHLKTHTGSQKPFQCRICMRNFSLRSILKAHTRTHTGEK | |
| OFF(mil) | GTGAA | PFQCRICMRNFSQAGNLSRHLRTHTGGGGSQKPFQCRICMRNFSL | |
| | | GENLRRHLRTHTGEKPFQCRICMRNFSRGDALARHLKTHLRGS | |
| CMV:ZFP | TAGGACC | SRPGERPFQCRICMRNFSTTYHLIRHTRTHTGEKPFQCRICMRNFS | 2109 |
| 781- | CAGCAGG | ENSKLKRHLRTHTGSQKPFQCRICMRNFSKKENLLQHTRTHTGEK | |
| OFF(mil) | GCGTG | PFQCRICMRNFSRQDNLNSHLRTHTGGGGSQKPFQCRICMRNFSD | |
| | | GSNLRRHLRTHTGEKPFQCRICMRNFSRIDNLDGHLKTHLRGS | |
| CMV:ZFP | GACAGGT | SRPGERPFQCRICMRNFSKKFNLLQHTRTHTGEKPFQCRICMRNF | 2110 |
| 789- | AGGACCC | SRKDYLISHLRTHTGGGGSQKPFQCRICMRNFSDRGNLTRHLRTH | |
| OFF(mil) | AGCAG | TGEKPFQCRICMRNFSRKTGLLIHLKTHTGSQKPFQCRICMRNFSR | |
| | | RAHLLNHTRTHTGEKPFQCRICMRNFSDRGNLTRHLRTHLRGS | |
| CMV:ZFP | GGGGAGG | SRPGERPFQCRICMRNFSDRSVLKRHLRTHTGEKPFQCRICMRNF | 2111 |
| 797- | GCAGCAT | SDKRSLAPHLKTHTGSQKPFQCRICMRNFSQAETLKRHLRTHTGE | |
| OFF(mil) | CCGCC | KPFQCRICMRNFSQGGTLRRHLKTHTGGGGSQKPFQCRICMRNFS | |
| | | REDNLDRHLRTHTGEKPFQCRICMRNFSRRHGLGRHLKTHLRGS | |
| CMV:ZFP | GAGGCTG | SRPGERPFQCRICMRNFSRQDNLGRHLRTHTGEKPFQCRICMRNF | 2112 |
| 809- | TGTGCTTC | SQRNTLKGHLKTHTGGGGSQKPFQCRICMRNFSRRRNLTLHTRT | |
| OFF(mil) | TGAG | HTGEKPFQCRICMRNFSRVEVLTNHLRTHTGSQKPFQCRICMRNF | |
| | | SVRETLVRHLRTHTGEKPFQCRICMRNFSRGDNLKRHLKTHLRGS | |
| CMV:ZFP | GAAAATG | SRPGERPFQCRICMRNFSDRTTLKRHLRTHTGEKPFQCRICMRNFS | 2113 |
| 573- | ACAGGTG | RVDHLKTHLKTHTGSQKPFQCRICMRNFSRSTHLHNHTRTHTGE | |
| OFF(mil) | GGCC | KPFQCRICMRNFSDRGNLTRHLRTHTGSQKPFQCRICMRNFSLAW | |
| | | NLNTHLRTHTGEKPFQCRICMRNFSQSVNLRRHLKTHLRGS | |
| CMV:ZFP | TTGGTGTG | SRPGERPFQCRICMRNFSQSTTLKRHLRTHTGEKPFQCRICMRNFS | 2114 |
| 581- | GGAGTAG | RRDGLAGHLKTHTGSQKPFQCRICMRNFSRGDNLGRHLRTHTGE | |
| OFF(mil) | GCA | KPFQCRICMRNFSRSDHLSLHLKTHTGSQKPFQCRICMRNFSRKE | |
| | | SLTIHLRTHTGEKPFQCRICMRNFSRMDVLSLHLKTHLRGS | |
| CMV:ZFP | GGTCAGG | SRPGERPFQCRICMRNFSQLSNLTRHTRTHTGEKPFQCRICMRNFS | 2115 |
| 592- | GGACGGG | RREHLVRHLRTHTGSQKPFQCRICMRNFSKNNDLTRHTRTHTGE | |
| OFF(mil) | GGAA | KPFQCRICMRNFSRREHLVRHLRTHTGSQKPFQCRICMRNFSKGY | |
| | | NLTVHTRTHTGEKPFQCRICMRNFSHQHRLKTHLRTHLRGS | |
| CMV:ZFP | GGCTGGG | SRPGERPFQCRICMRNFSDSSVLRRHLRTHTGEKPFQCRICMRNFS | 2116 |
| 601- | AAGGGTG | QPHGLRGHLKTHTGSQKPFQCRICMRNFSRREHLVRHLRTHTGE | |
| OFF(mil) | TGCC | KPFQCRICMRNFSQDGNLGRHLKTHTGSQKPFQCRICMRNFSRRE | |
| | | HLTIHLRTHTGEKPFQCRICMRNFSEKSHLTRHLKTHLRGS | |
| CMV:ZFP | GTTGGAG | SRPGERPFQCRICMRNFSDRSSLKRHLRTHTGEKPFQCRICMRNFS | 2117 |
| 610- | GGGGAGA | QHPNLTRHLKTHTGSQKPFQCRICMRNFSQSPHLKRHLRTHTGEK | |
| OFF(mil) | AGTC | PFQCRICMRNFSRTEHLARHLKTHTGSQKPFQCRICMRNFSSRST | |
| | | HLRHTRTHTGEKPFQCRICMRNFSSHKSSLTHLRTHLRGS | |
| CMV:ZFP | CTGGAAT | SRPGERPFQCRICMRNFSKKFNLQAHTRTHTGEKPFQCRICMRNF | 2118 |
| 619- | GGCAGGA | SDPSNLARHLRTHTGSQKPFQCRICMRNFSRQDNLQSHLRTHTGE | |
| OFF(mil) | CCAG | KPFQCRICMRNFSRRHRLTLHLKTHTGSQKPFQCRICMRNFSQRS | |
| | | NLARHTRTHTGEKPFQCRICMRNFSRKESLDVHLRTHLRGS | |
| CMV:ZFP | GCCAACG | SRPGERPFQCRICMRNFSDLSTLRRHTRTHTGEKPFQCRICMRNFS | 2119 |
| 629- | GTGTAGC | LSQTLKRHLRTHTGSQKPFQCRICMRNFSQRSSLVRHLRTHTGEK | |
| OFF(mil) | TGCC | PFQCRICMRNFSQPHHLPRHLKTHTGSQKPFQCRICMRNFSGHTA | |
| | | LRNHTRTHTGEKPFQCRICMRNFSDSGVLKRHLRTHLRGS | |
| CMV:ZFP | GGAGTCT | SRPGERPFQCRICMRNFSTHAHLTRHTRTHTGEKPFQCRICMRNF | 2120 |
| 637- | GAGAAAA | SRRDNLHTHLRTHTGSQKPFQCRICMRNFSLRANLQRHTRTHTGE | |
| OFF(mil) | GGGA | KPFQCRICMRNFSQREHLNVHLRTHTGSQKPFQCRICMRNFSDHS | |
| | | SLKRHLRTHTGEKPFQCRICMRNFSQSPHLQRHLKTHLRGS | |
| CMV:ZFP | GCAATTT | SRPGERPFQCRICMRNFSQSSSLVRHLRTHTGEKPFQCRICMRNFS | 2121 |
| 648- | GGTGTGG | QTTHLSRHLKTHTGSQKPFQCRICMRNFSRREVLENHLRTHTGEK | |
| OFF(mil) | GAGTA | PFQCRICMRNFSVKHGLTRHLKTHTGGGGSQKPFQCRICMRNFSH | |
| | | RESLTVHLRTHTGEKPFQCRICMRNFSQTATLKRHLKTHLRGS | |
| CMV:ZFP | GAGGCAT | SRPGERPFQCRICMRNFSRREHLVRHLRTHTGEKPFQCRICMRNF | 2122 |
| 659- | CTGGGCG | SQNSHLRRHLKTHTGGGGSQKPFQCRICMRNFSRREHLMRHLRT | |
| OFF(mil) | GAGGG | HTGEKPFQCRICMRNFSARNTLKGHLKTHTGSQKPFQCRICMRNF | |
| | | SQGGTLNRHLRTHTGEKPFQCRICMRNFSRRDHLLRHLKTHLRGS | |
| CMV:ZFP | GAAAATG | SRPGERPFQCRICMRNFSDSSVLRRHLRTHTGEKPFQCRICMRNFS | 2123 |
| 574- | ACAGGTG | RSDHLSLHLKTHTGSQKPFQCRICMRNFSRRAHLLNHTRTHTGEK | |
| OFF(mil) | GGCC | PFQCRICMRNFSDRGNLTRHLRTHTGSQKPFQCRICMRNFSLAWN | |
| | | LNTHLRTHTGEKPFQCRICMRNFSQSVNLRRHLKTHLRGS | |
| CMV:ZFP | GGGTGTT | SRPGERPFQCRICMRNFSQRGNLTRHLRTHTGEKPFQCRICMRNF | 2124 |
| 582- | CAGAAAC | SQSNSLKYHLKTHTGSQKPFQCRICMRNFSQRGNLLRHLRTHTGE | |
| OFF(mil) | AGAA | KPFQCRICMRNFSTKQWTLGHLKTHTGSQKPFQCRICMRNFSRR | |
| | | QHLQYHTRTHTGEKPFQCRICMRNFSRREHLVRHLRTHLRGS | |
| CMV:ZFP | GGTCAGG | SRPGERPFQCRICMRNFSQLSNLTRHTRTHTGEKPFQCRICMRNFS | 2125 |
| 593- | GGACGGG | RREHLVRHLRTHTGSQKPFQCRICMRNFSKGNDLTRHTRTHTGE | |
| OFF(mil) | GGAA | KPFQCRICMRNFSRREHL VRHLRTHTGSQKPFQCRICMRNFSKRF | |
| | | NLMEHTRTHTGEKPFQCRICMRNFSHRHSLTRHLRTHLRGS | |
| CMV:ZFP | GGCTGGG | SRPGERPFQCRICMRNFSDSSVLRRHLRTHTGEKPFQCRICMRNFS | 2126 |
| 602- | AAGGGTG | QPHGLRGHLKTHTGSQKPFQCRICMRNFSRREHLVRHLRTHTGE | |
| OFF(mil) | TGCC | KPFQCRICMRNFSQDGNLGRHLKTHTGSQKPFQCRICMRNFSRKE | |
| | | HLSIHLRTHTGEKPFQCRICMRNFSDKSHLTRHLKTHLRGS | |
| CMV:ZFP | GTTGGAG | SRPGERPFQCRICMRNFSDRSSLKRHLRTHTGEKPFQCRICMRNFS | 2127 |
| 611- | GGGGAGA | QHPNLTRHLKTHTGSQKPFQCRICMRNFSQSAHLKRHLRTHTGE | |
| OFF(mil) | AGTC | KPFQCRICMRNFSRTEHLARHLKTHTGSQKPFQCRICMRNFSQSA | |
| | | HLKRHLRTHTGEKPFQCRICMRNFSHHNSLTRHLKTHLRGS | |
| CMV:ZFP | CTGGAAT | SRPGERPFQCRICMRNFSKKCNLLSHTRTHTGEKPFQCRICMRNFS | 2128 |
| 621- | GGCAGGA | ERGNLARHLRTHTGSQKPFQCRICMRNFSRKDNLNTHLRTHTGE | |
| OFF(mil) | CCAG | KPFQCRICMRNFSRRHRLTLHLKTHTGSQKPFQCRICMRNFSQGG | |
| | | NLTRHLRTHTGEKPFQCRICMRNFSRKMTLMAHLKTHLRGS | |
| CMV:ZFP | GCCAACG | SRPGERPFQCRICMRNFSDGSTLRRHTRTHTGEKPFQCRICMRNFS | 2129 |
| 630- | GTGTAGC | LSQTLKRHLRTHTGSQKPFQCRICMRNFSQRSSLVRHLRTHTGEK | |
| OFF(mil) | TGCC | PFQCRICMRNFSLKHGLTRHLKTHTGSQKPFQCRICMRNFSGRTA | |
| | | LKTHTRTHTGEKPFQCRICMRNFSDSGVLKRHLRTHLRGS | |
| CMV:ZFP | GCATGAG | SRPGERPFQCRICMRNFSRREVLENHLRTHTGEKPFQCRICMRNFS | 2130 |
| 641- | ACTTTGA | RRDNLNRHLKTHTGSQKPFQCRICMRNFSGMLSLAVHTRTHTGE | |
| OFF(mil) | GGTG | KPFQCRICMRNFSDASNLRRHLRTHTGSQKPFQCRICMRNFSQNE | |
| | | HLKVHLRTHTGEKPFQCRICMRNFSQGGTLRRHLKTHLRGS | |
| CMV:ZFP | GAAGTCC | SRPGERPFQCRICMRNFSHRQTLTRHTRTHTGEKPFQCRICMRNFS | 2131 |
| 651- | CCAGCAG | RQDNLGRHLRTHTGSQKPFQCRICMRNFSQSGTLKRHLRTHTGE | |
| OFF(mil) | AGGCT | KPFQCRICMRNFSKQWYLQLHLKTHTGGGGSQKPFQCRICMRNF | |
| | | SDRSSLKRHLRTHTGEKPFQCRICMRNFSQHPNLTRHLKTHLRGS | |
| CMV:ZFP | GAAAATG | SRPGERPFQCRICMRNFSDRTTLKRHLRTHTGEKPFQCRICMRNFS | 2132 |
| 575- | ACAGGTG | RVDHLKTHLKTHTGSQKPFQCRICMRNFSRRTHLRVHTRTHTGE | |
| OFF(mil) | GGCC | KPFQCRICMRNFSDRGNLTRHLRTHTGSQKPFQCRICMRNFSHQ | |
| | | WNLQTHLRTHTGEKPFQCRICMRNFSQDGNLGRHLKTHLRGS | |
| CMV:ZFP | GGGTGTT | SRPGERPFQCRICMRNFSQRGNLTRHLRTHTGEKPFQCRICMRNF | 2133 |
| 584- | CAGAAAC | SQSNSLKYHLKTHTGSQKPFQCRICMRNFSQRGNLARHLRTHTG | |
| OFF(mil) | AGAA | EKPFQCRICMRNFSTKQWTLGHLKTHTGSQKPFQCRICMRNFSRR | |
| | | QHLQYHTRTHTGEKPFQCRICMRNFSRREHLVRHLRTHLRGS | |
| CMV:ZFP | GGTCAGG | SRPGERPFQCRICMRNFSTTTNLRRHTRTHTGEKPFQCRICMRNFS | 2134 |
| 594- | GGACGGG | RREHLVRHLRTHTGSQKPFQCRICMRNFSRRHDLRRHTRTHTGE | |
| OFF(mil) | GGAA | KPFQCRICMRNFSRQAHLQNHLRTHTGSQKPFQCRICMRNFSKRF | |
| | | NLMEHTRTHTGEKPFQCRICMRNFSHRHSLTRHLRTHLRGS | |
| CMV:ZFP | GCCGGTG | SRPGERPFQCRICMRNFSRSRNLTLHTRTHTGEKPFQCRICMRNFS | 2135 |
| 603- | TGGTTGG | RREHLVRHLRTHTGSQKPFQCRICMRNFSHKSSLTRHLRTHTGEK | |
| OFF(mil) | GTGC | PFQCRICMRNFSRNTALQHHLKTHTGSQKPFQCRICMRNFSLGHH | |
| | | LVRHLRTHTGEKPFQCRICMRNFSDPSNLRRHLKTHLRGS | |
| CMV:ZFP | GAGGACC | SRPGERPFQCRICMRNFSYKESLVRHLRTHTGEKPFQCRICMRNFS | 2136 |
| 612- | CAGCAGG | QGGHLARHLKTHTGSQKPFQCRICMRNFSQSGTLKRHLRTHTGE | |
| OFF(mil) | AATC | KPFQCRICMRNFSKQWYLQLHLKTHTGSQKPFQCRICMRNFSEE | |
| | | ANLRRHTRTHTGEKPFQCRICMRNFSRQDNLGRHLRTHLRGS | |
| CMV:ZFP | CTGGAAT | SRPGERPFQCRICMRNFSSRFNLSTHTRTHTGEKPFQCRICMRNFS | 2137 |
| 622- | GGCAGGA | DASNLRRHLRTHTGSQKPFQCRICMRNFSRKDNLNTHLRTHTGE | |
| OFF(mil) | CCAG | KPFQCRICMRNFSRTAHLQVHLKTHTGSQKPFQCRICMRNFSQRS | |
| | | NLARHTRTHTGEKPFQCRICMRNFSRKESLDVHLRTHLRGS | |
| CMV:ZFP | GCCAACG | SRPGERPFQCRICMRNFSDGSTLRRHTRTHTGEKPFQCRICMRNFS | 2138 |
| 631- | GTGTAGC | LSQTLKRHLRTHTGSQKPFQCRICMRNFSQRSSLVRHLRTHTGEK | |
| OFF(mil) | TGCC | PFQCRICMRNFSQPHHLPRHLKTHTGSQKPFQCRICMRNFSGHTA | |
| | | LRNHTRTHTGEKPFQCRICMRNFSDSGVLKRHLRTHLRGS | |
| CMV:ZFP | GATACTG | SRPGERPFQCRICMRNFSQGTNLVRHLRTHTGEKPFQCRICMRNF | 2139 |
| 642- | TTGACTGT | SQHSSLSRHLKTHTGGGGSQKPFQCRICMRNFSDRGNLTRHLRTH | |
| OFF(mil) | AGAA | TGEKPFQCRICMRNFSIRTSLKRHLKTHTGSQKPFQCRICMRNFSH | |
| | | KESLKVHLRTHTGEKPFQCRICMRNFSVMGNLTRHLKTHLRGS | |
| CMV:ZFP | GAAGTCC | SRPGERPFQCRICMRNFSTKPILVRHTRTHTGEKPFQCRICMRNFS | 2140 |
| 652- | CCAGCAG | RQDNLGRHLRTHTGSQKPFQCRICMRNFSQSGTLKRHLRTHTGE | |
| OFF(mil) | AGGCT | KPFQCRICMRNFSKQWYLQLHLKTHTGGGGSQKPFQCRICMRNF | |
| | | SDPSSLKRHLRTHTGEKPFQCRICMRNFSQHPNLTRHLKTHLRGS | |
| CMV:ZFP | GAAAATG | SRPGERPFQCRICMRNFSDRTTLKRHLRTHTGEKPFQCRICMRNFS | 2141 |
| 576- | ACAGGTG | RSDHLSLHLKTHTGSQKPFQCRICMRNFSRRTHLRVHTRTHTGEK | |
| OFF(mil) | GGCC | PFQCRICMRNFSDRGNLTRHLRTHTGSQKPFQCRICMRNFSLPWN | |
| | | LQTHLRTHTGEKPFQCRICMRNFSQDGNLGRHLKTHLRGS | |
| CMV:ZFP | GGGTGTT | SRPGERPFQCRICMRNFSQQTNLTRHLRTHTGEKPFQCRICMRNF | 2142 |
| 586- | CAGAAAC | SQGGILYRHLKTHTGSQKPFQCRICMRNFSQAGNLSRHLRTHTGE | |
| OFF(mil) | AGAA | KPFQCRICMRNFSTKQWTLGHLKTHTGSQKPFQCRICMRNFSRR | |
| | | QHLQYHTRTHTGEKPFQCRICMRNFSRREHLVRHLRTHLRGS | |
| CMV:ZFP | GGTCAGG | SRPGERPFQCRICMRNFSTTTNLRRHTRTHTGEKPFQCRICMRNFS | 2143 |
| 595- | GGACGGG | RREHLVRHLRTHTGSQKPFQCRICMRNFSRRHDLRRHTRTHTGE | |
| OFF(mil) | GGAA | KPFQCRICMRNFSRQAHLQNHLRTHTGSQKPFQCRICMRNFSKRF | |
| | | NLNQHTRTHTGEKPFQCRICMRNFSHKHRLVTHLRTHLRGS | |
| CMV:ZFP | GCCGGTG | SRPGERPFQCRICMRNFSRRRNLTLHTRTHTGEKPFQCRICMRNFS | 2144 |
| 604- | TGGTTGG | RREHLVRHLRTHTGSQKPFQCRICMRNFSTSTVLMRHTRTHTGE | |
| OFF(mil) | GTGC | KPFQCRICMRNFSRREVLENHLRTHTGSQKPFQCRICMRNFSLRQ | |
| | | HLVRHLRTHTGEKPFQCRICMRNFSDPSNLRRHLKTHLRGS | |
| CMV:ZFP | GAGGACC | SRPGERPFQCRICMRNFSYKESLVRHLRTHTGEKPFQCRICMRNFS | 2145 |
| 613- | CAGCAGG | QGGHLARHLKTHTGSQKPFQCRICMRNFSQSGTLKRHLRTHTGE | |
| OFF(mil) | AATC | KPFQCRICMRNFSKQWYLQLHLKTHTGSQKPFQCRICMRNFSEE | |
| | | VNLRRHTRTHTGEKPFQCRICMRNFSRADNLGRHLRTHLRGS | |
| CMV:ZFP | CTGGAAT | SRPGERPFQCRICMRNFSSRFNLSTHTRTHTGEKPFQCRICMRNFS | 2146 |
| 623- | GGCAGGA | DASNLRRHLRTHTGSQKPFQCRICMRNFSRKDNLNTHLRTHTGE | |
| OFF(mil) | CCAG | KPFQCRICMRNFSRRHRLTLHLKTHTGSQKPFQCRICMRNFSQRG | |
| | | NLARHLRTHTGEKPFQCRICMRNFSRNHSLRMHLKTHLRGS | |
| CMV:ZFP | GCCAACG | SRPGERPFQCRICMRNFSDPSTLRRHTRTHTGEKPFQCRICMRNFS | 2147 |
| 632- | GTGTAGC | LRDSLKRHLRTHTGSQKPFQCRICMRNFSQRSSLVRHLRTHTGEK | |
| OFF(mil) | TGCC | PFQCRICMRNFSQPHHLPRHLKTHTGSQKPFQCRICMRNFSGASA | |
| | | LRSHTRTHTGEKPFQCRICMRNFSESTTLKRHLRTHLRGS | |
| CMV:ZFP | GATACTG | SRPGERPFQCRICMRNFSQGTNLVRHLRTHTGEKPFQCRICMRNF | 2148 |
| 643- | TTGACTGT | SQHSSLSRHLKTHTGGGGSQKPFQCRICMRNFSDRGNLTRHLRTH | |
| OFF(mil) | AGAA | TGEKPFQCRICMRNFSHHNSLTRHLKTHTGSQKPFQCRICMRNFS | |
| | | HKESLKVHLRTHTGEKPFQCRICMRNFSVMGNLTRHLKTHLRGS | |
| CMV:ZFP | GAAGTCC | SRPGERPFQCRICMRNFSTKPILVRHTRTHTGEKPFQCRICMRNFS | 2149 |
| 653- | CCAGCAG | RQDNLGRHLRTHTGSQKPFQCRICMRNFSQSGTLKRHLRTHTGE | |
| OFF(mil) | AGGCT | KPFQCRICMRNFSKQWYLQLHLKTHTGGGGSQKPFQCRICMRNF | |
| | | SDRSSLKRHLRTHTGEKPFQCRICMRNFSQHPNLTRHLKTHLRGS | |
| CMV:ZFP | GAAAATG | SRPGERPFQCRICMRNFSDRTTLKRHLRTHTGEKPFQCRICMRNFS | 2150 |
| 577- | ACAGGTG | RVDHLKTHLKTHTGSQKPFQCRICMRNFSRRTHLRVHTRTHTGE | |
| OFF(mil) | GGCC | KPFQCRICMRNFSDRGNLTRHLRTHTGSQKPFQCRICMRNFSLPW | |
| | | NLQTHLRTHTGEKPFQCRICMRNFSQDGNLGRHLKTHLRGS | |
| CMV:ZFP | GGGTGTT | SRPGERPFQCRICMRNFSQQTNLTRHLRTHTGEKPFQCRICMRNF | 2151 |
| 587- | CAGAAAC | SQGGILYRHLKTHTGSQKPFQCRICMRNFSQRGNLLRHLRTHTGE | |
| OFF(mil) | AGAA | KPFQCRICMRNFSTKQWTLGHLKTHTGSQKPFQCRICMRNFSRR | |
| | | QHLQYHTRTHTGEKPFQCRICMRNFSRREHLVRHLRTHLRGS | |
| CMV:ZFP | GGTCAGG | SRPGERPFQCRICMRNFSQLSNLTRHTRTHTGEKPFQCRICMRNFS | 2152 |
| 596- | GGACGGG | RREHLVRHLRTHTGSQKPFQCRICMRNFSRRHDLRRHTRTHTGE | |
| OFF(mil) | GGAA | KPFQCRICMRNFSRQAHLQNHLRTHTGSQKPFQCRICMRNFSKG | |
| | | YNLTVHTRTHTGEKPFQCRICMRNFSHQHRLKTHLRTHLRGS | |
| CMV:ZFP | GCCGGTG | SRPGERPFQCRICMRNFSRSRNLTLHTRTHTGEKPFQCRICMRNFS | 2153 |
| 605- | TGGTTGG | RREHLVRHLRTHTGSQKPFQCRICMRNFSHKSSLTRHLRTHTGEK | |
| OFF(mil) | GTGC | PFQCRICMRNFSRNTALQHHLKTHTGSQKPFQCRICMRNFSLRQH | |
| | | LVRHLRTHTGEKPFQCRICMRNFSDPSNLRRHLKTHLRGS | |
| CMV:ZFP | GAGGACC | SRPGERPFQCRICMRNFSYKESLVRHLRTHTGEKPFQCRICMRNFS | 2154 |
| 614- | CAGCAGG | QGGHLARHLKTHTGSQKPFQCRICMRNFSQSGTLKRHLRTHTGE | |
| OFF(mil) | AATC | KPFQCRICMRNFSKQWYLQLHLKTHTGSQKPFQCRICMRNFSDR | |
| | | ANLRRHLRTHTGEKPFQCRICMRNFSRADNLGRHLKTHLRGS | |
| CMV:ZFP | GGAGTGG | SRPGERPFQCRICMRNFSVRSRLRLHTRTHTGEKPFQCRICMRNFS | 2155 |
| 624- | AGTCTGG | EKGHLNRHLRTHTGSQKPFQCRICMRNFSSKPNLKMHTRTHTGE | |
| OFF(mil) | CAGA | KPFQCRICMRNFSRGDNLGRHLRTHTGSQKPFQCRICMRNFSRRE | |
| | | VLENHLRTHTGEKPFQCRICMRNFSQKPHLSRHLKTHLRGS | |
| CMV:ZFP | GGAGTCT | SRPGERPFQCRICMRNFSTHAHLTRHTRTHTGEKPFQCRICMRNF | 2156 |
| 633- | GAGAAAA | SRQDNLHTHLRTHTGSQKPFQCRICMRNFSLRANLQRHTRTHTG | |
| OFF(mil) | GGGA | EKPFQCRICMRNFSQREHLNVHLRTHTGSQKPFQCRICMRNFSTS | |
| | | TLLNRHTRTHTGEKPFQCRICMRNFSQSPHLKRHLRTHLRGS | |
| CMV:ZFP | GATACTG | SRPGERPFQCRICMRNFSQAQNLSRHLRTHTGEKPFQCRICMRNF | 2157 |
| 644- | TTGACTGT | SQKIVLLRHLKTHTGGGGSQKPFQCRICMRNFSDRGNLTRHLRTH | |
| OFF(mil) | AGAA | TGEKPFQCRICMRNFSIRTSLKRHLKTHTGSQKPFQCRICMRNFSH | |
| | | KESLKVHLRTHTGEKPFQCRICMRNFSVMGNLTRHLKTHLRGS | |
| CMV:ZFP | GAGGCAT | SRPGERPFQCRICMRNFSRREHLMRHLRTHTGEKPFQCRICMRNF | 2158 |
| 654- | CTGGGCG | SQTTHLSRHLKTHTGGGGSQKPFQCRICMRNFSRREHLVRHLRTH | |
| OFF(mil) | GAGGG | TGEKPFQCRICMRNFSQRNTLLNHLKTHTGSQKPFQCRICMRNFS | |
| | | QGGTLNRHLRTHTGEKPFQCRICMRNFSRRDHLLRHLKTHLRGS | |
| CMV:ZFP | GAAAATG | SRPGERPFQCRICMRNFSDRTTLKRHLRTHTGEKPFQCRICMRNFS | 2159 |
| 578- | ACAGGTG | RSDHLSLHLKTHTGSQKPFQCRICMRNFSRSTHLHNHTRTHTGEK | |
| OFF(mil) | GGCC | PFQCRICMRNFSDRGNLTRHLRTHTGSQKPFQCRICMRNFSHQW | |
| | | NLQTHLRTHTGEKPFQCRICMRNFSQDGNLGRHLKTHLRGS | |
| CMV:ZFP | GGGGAAG | SRPGERPFQCRICMRNFSKKIDLVRHTRTHTGEKPFQCRICMRNFS | 2160 |
| 588- | CTGAGGG | LKGHLTRHLRTHTGSQKPFQCRICMRNFSRQDNLGRHLRTHTGE | |
| OFF(mil) | CACG | KPFQCRICMRNFSVSNTLTRHLKTHTGSQKPFQCRICMRNFSQTA | |
| | | NLMRHLRTHTGEKPFQCRICMRNFSRTEHLARHLKTHLRGS | |
| CMV:ZFP | GGCTGGG | SRPGERPFQCRICMRNFSDSSVLRRHLRTHTGEKPFQCRICMRNFS | 2161 |
| 597- | AAGGGTG | QPHGLAHHLKTHTGSQKPFQCRICMRNFSRREHLVRHLRTHTGE | |
| OFF(mil) | TGCC | KPFQCRICMRNFSQDGNLGRHLKTHTGSQKPFQCRICMRNFSRKE | |
| | | HLSIHLRTHTGEKPFQCRICMRNFSDKSHLTRHLKTHLRGS | |
| CMV:ZFP | TAAGTTG | SRPGERPFQCRICMRNFSRRDNLHTHLRTHTGEKPFQCRICMRNF | 2162 |
| 607- | CTACGGG | SQGGHLKRHLKTHTGSQKPFQCRICMRNFSRNITLVRHTRTHTGE | |
| OFF(mil) | AAAG | KPFQCRICMRNFSVKETLVRHLRTHTGSQKPFQCRICMRNFSHKS | |
| | | SLTRHLRTHTGEKPFQCRICMRNFSQQGNLQLHLKTHLRGS | |
| CMV:ZFP | GACAGGT | SRPGERPFQCRICMRNFSQSGTLKRHLRTHTGEKPFQCRICMRNFS | 2163 |
| 615- | AGGACCC | KQWYLQLHLKTHTGSQKPFQCRICMRNFSDPSNLQRHLRTHTGE | |
| OFF(mil) | AGCA | KPFQCRICMRNFSRRDNLPKHLKTHTGSQKPFQCRICMRNFSRKD | |
| | | HLKTHLRTHTGEKPFQCRICMRNFSEGGNLMRHLKTHLRGS | |
| CMV:ZFP | GGAGTGG | SRPGERPFQCRICMRNFSRRSRLTLHTRTHTGEKPFQCRICMRNFS | 2164 |
| 625- | AGTCTGG | DSGHLKRHLRTHTGSQKPFQCRICMRNFSSKPNLKMHTRTHTGE | |
| OFF(mil) | CAGA | KPFQCRICMRNFSRGDNLGRHLRTHTGSQKPFQCRICMRNFSRRE | |
| | | VLENHLRTHTGEKPFQCRICMRNFSQSAHLGRHLKTHLRGS | |
| CMV:ZFP | GGAGTCT | SRPGERPFQCRICMRNFSTHAHLTRHTRTHTGEKPFQCRICMRNF | 2165 |
| 634- | GAGAAAA | SRQDNLHTHLRTHTGSQKPFQCRICMRNFSQNANLKRHTRTHTG | |
| OFF(mil) | GGGA | EKPFQCRICMRNFSQREHLTTHLRTHTGSQKPFQCRICMRNFSDH | |
| | | SSLKRHLRTHTGEKPFQCRICMRNFSQSPHLQRHLKTHLRGS | |
| CMV:ZFP | GACTGTA | SRPGERPFQCRICMRNFSRQDNLGRHLRTHTGEKPFQCRICMRNF | 2166 |
| 645- | GAAGGCT | SQRNALAGHLKTHTGSQKPFQCRICMRNFSSPSKLARHTRTHTGE | |
| OFF(mil) | CTGAG | KPFQCRICMRNFSQAGNLSRHLRTHTGGGGSQKPFQCRICMRNFS | |
| | | RRQHLELHTRTHTGEKPFQCRICMRNFSDRGNLTRHLRTHLRGS | |
| CMV:ZFP | GAGGCAT | SRPGERPFQCRICMRNFSKKDHLHRHTRTHTGEKPFQCRICMRNF | 2167 |
| 655- | CTGGGCG | SQSAHLKRHLRTHTGGGGSQKPFQCRICMRNFSRREHLMRHLRT | |
| OFF(mil) | GAGGG | HTGEKPFQCRICMRNFSARNTLKGHLKTHTGSQKPFQCRICMRNF | |
| | | SQSGTLHRHLRTHTGEKPFQCRICMRNFSRRDHLLRHLKTHLRGS | |
| CMV:ZFP | TTGGTGTG | SRPGERPFQCRICMRNFSQSTTLKRHLRTHTGEKPFQCRICMRNFS | 2168 |
| 579- | GGAGTAG | RRDGLAGHLKTHTGSQKPFQCRICMRNFSRNHNLERHTRTHTGE | |
| OFF(mil) | GCA | KPFQCRICMRNFSRREHLTIHLRTHTGSQKPFQCRICMRNFSRRES | |
| | | LTIHLRTHTGEKPFQCRICMRNFSRMDVLSLHLKTHLRGS | |
| CMV:ZFP | GGGGAAG | SRPGERPFQCRICMRNFSKKIDLVRHTRTHTGEKPFQCRICMRNFS | 2169 |
| 589- | CTGAGGG | LKGHLTRHLRTHTGSQKPFQCRICMRNFSRQDNLGRHLRTHTGE | |
| OFF(mil) | CACG | KPFQCRICMRNFSVSNTLTRHLKTHTGSQKPFQCRICMRNFSQTQ | |
| | | NLTRHLRTHTGEKPFQCRICMRNFSRTEHLARHLKTHLRGS | |
| CMV:ZFP | GGCTGGG | SRPGERPFQCRICMRNFSDSSVLRRHLRTHTGEKPFQCRICMRNFS | 2170 |
| 598- | AAGGGTG | QPHGLRGHLKTHTGSQKPFQCRICMRNFSRQEHLVRHLRTHTGE | |
| OFF(mil) | TGCC | KPFQCRICMRNFSQHPNLTRHLKTHTGSQKPFQCRICMRNFSRKE | |
| | | HLSIHLRTHTGEKPFQCRICMRNFSDKSHLTRHLKTHLRGS | |
| CMV:ZFP | TAAGTTG | SRPGERPFQCRICMRNFSRRDNLHTHLRTHTGEKPFQCRICMRNF | 2171 |
| 608- | CTACGGG | SQGGHLKRHLKTHTGSQKPFQCRICMRNFSRQVTLTRHTRTHTG | |
| OFF(mil) | AAAG | EKPFQCRICMRNFSVRETLVRHLRTHTGSQKPFQCRICMRNFSHK | |
| | | SSLTRHLRTHTGEKPFQCRICMRNFSQSGNLHTHLKTHLRGS | |
| CMV:ZFP | GACAGGT | SRPGERPFQCRICMRNFSQSGTLKRHLRTHTGEKPFQCRICMRNFS | 2172 |
| 616- | AGGACCC | KQWYLQLHLKTHTGSQKPFQCRICMRNFSDPSNLQRHLRTHTGE | |
| OFF(mil) | AGCA | KPFQCRICMRNFSRRDNLPKHLKTHTGSQKPFQCRICMRNFSRRA | |
| | | HLLNHTRTHTGEKPFQCRICMRNFSDRGNLTRHLRTHLRGS | |
| CMV:ZFP | GCCAACG | SRPGERPFQCRICMRNFSDPSTLRRHTRTHTGEKPFQCRICMRNFS | 2173 |
| 627- | GTGTAGC | LRDSLKRHLRTHTGSQKPFQCRICMRNFSQRSSLVRHLRTHTGEK | |
| OFF(mil) | TGCC | PFQCRICMRNFSLKHGLTRHLKTHTGSQKPFQCRICMRNFSGHTA | |
| | | LRNHTRTHTGEKPFQCRICMRNFSDSGVLKRHLRTHLRGS | |
| CMV:ZFP | GGAGTCT | SRPGERPFQCRICMRNFSTAAHLTRHTRTHTGEKPFQCRICMRNF | 2174 |
| 635- | GAGAAAA | SRQDNLHTHLRTHTGSQKPFQCRICMRNFSLRANLQRHTRTHTG | |
| OFF(mil) | GGGA | EKPFQCRICMRNFSQREHLNVHLRTHTGSQKPFQCRICMRNFSTS | |
| | | TLLNRHTRTHTGEKPFQCRICMRNFSQSPHLKRHLRTHLRGS | |
| CMV:ZFP | GACTGTA | SRPGERPFQCRICMRNFSRQDNLGRHLRTHTGEKPFQCRICMRNF | 2175 |
| 646- | GAAGGCT | SQRNTLKGHLKTHTGSQKPFQCRICMRNFSTPSKLDRHTRTHTGE | |
| OFF(mil) | CTGAG | KPFQCRICMRNFSQMSNLDRHLRTHTGGGGSQKPFQCRICMRNF | |
| | | SRKQHLTLHTRTHTGEKPFQCRICMRNFSDRGNLTRHLRTHLRGS | |
| CMV:ZFP | GAGGCAT | SRPGERPFQCRICMRNFSRREHLMRHLRTHTGEKPFQCRICMRNF | 2176 |
| 656- | CTGGGCG | SQTTHLSRHLKTHTGGGGSQKPFQCRICMRNFSRQEHLVRHLRT | |
| OFF(mil) | GAGGG | HTGEKPFQCRICMRNFSARNTLKGHLKTHTGSQKPFQCRICMRNF | |
| | | SQGGTLNRHLRTHTGEKPFQCRICMRNFSRRDHLLRHLKTHLRGS | |
| CMV:ZFP | TTGGTGTG | SRPGERPFQCRICMRNFSQSTTLKRHLRTHTGEKPFQCRICMRNFS | 2177 |
| 580- | GGAGTAG | RRDGLAGHLKTHTGSQKPFQCRICMRNFSRQDNLGRHLRTHTGE | |
| OFF(mil) | GCA | KPFQCRICMRNFSRMDHLAGHLKTHTGSQKPFQCRICMRNFSRK | |
| | | ESLTIHLRTHTGEKPFQCRICMRNFSRMDVLSLHLKTHLRGS | |
| CMV:ZFP | GGGGAAG | SRPGERPFQCRICMRNFSKKIDLVRHTRTHTGEKPFQCRICMRNFS | 2178 |
| 590- | CTGAGGG | LKGHLTRHLRTHTGSQKPFQCRICMRNFSRQDNLGRHLRTHTGE | |
| OFF(mil) | CACG | KPFQCRICMRNFSVSNTLSRHLKTHTGSQKPFQCRICMRNFSQTQ | |
| | | NLTRHLRTHTGEKPFQCRICMRNFSRTEHLARHLKTHLRGS | |
| CMV:ZFP | GGCTGGG | SRPGERPFQCRICMRNFSDSSVLRRHLRTHTGEKPFQCRICMRNFS | 2179 |
| 599- | AAGGGTG | QAHGLTAHLKTHTGSQKPFQCRICMRNFSRQEHLVRHLRTHTGE | |
| OFF(mil) | TGCC | KPFQCRICMRNFSQHPNLTRHLKTHTGSQKPFQCRICMRNFSRKE | |
| | | HLVGHLRTHTGEKPFQCRICMRNFSEKSHLTRHLKTHLRGS | |
| CMV:ZFP | GTTGGAG | SRPGERPFQCRICMRNFSDRSSLKRHLRTHTGEKPFQCRICMRNFS | 2180 |
| 609- | GGGGAGA | QHPNLTRHLKTHTGSQKPFQCRICMRNFSQSAHLKRHLRTHTGE | |
| OFF(mil) | AGTC | KPFQCRICMRNFSRTEHLARHLKTHTGSQKPFQCRICMRNFSRST | |
| | | HLRVHTRTHTGEKPFQCRICMRNFSHKSSLTRHLRTHLRGS | |
| CMV:ZFP | GACAGGT | SRPGERPFQCRICMRNFSQSGTLKRHLRTHTGEKPFQCRICMRNFS | 2181 |
| 617- | AGGACCC | KQWYLQLHLKTHTGSQKPFQCRICMRNFSDRGNLTRHLRTHTGE | |
| OFF(mil) | AGCA | KPFQCRICMRNFSRKTGLLIHLKTHTGSQKPFQCRICMRNFSRKD | |
| | | HLKTHLRTHTGEKPFQCRICMRNFSEGGNLMRHLKTHLRGS | |
| CMV:ZFP | GCCAACG | SRPGERPFQCRICMRNFSDGSTLRRHTRTHTGEKPFQCRICMRNFS | 2182 |
| 628- | GTGTAGC | LSQTLKRHLRTHTGSQKPFQCRICMRNFSQNSALHRHTRTHTGEK | |
| OFF(mil) | TGCC | PFQCRICMRNFSQRHHLDRHLRTHTGSQKPFQCRICMRNFSGRTA | |
| | | LKTHTRTHTGEKPFQCRICMRNFSDSGVLKRHLRTHLRGS | |
| CMV:ZFP | GGAGTCT | SRPGERPFQCRICMRNFSTHAHLTRHTRTHTGEKPFQCRICMRNF | 2183 |
| 636- | GAGAAAA | SRRDNLHTHLRTHTGSQKPFQCRICMRNFSLRANLQRHTRTHTGE | |
| OFF(mil) | GGGA | KPFQCRICMRNFSQREHLNVHLRTHTGSQKPFQCRICMRNFSTST | |
| | | LLNRHTRTHTGEKPFQCRICMRNFSQSPHLKRHLRTHLRGS | |
| CMV:ZFP | GACTGTA | SRPGERPFQCRICMRNFSRQDNLGRHLRTHTGEKPFQCRICMRNF | 2184 |
| 647- | GAAGGCT | SQRNALAGHLKTHTGSQKPFQCRICMRNFSSPSKLARHTRTHTGE | |
| OFF(mil) | CTGAG | KPFQCRICMRNFSQMSNLDRHLRTHTGGGGSQKPFQCRICMRNF | |
| | | SRKQHLQLHTRTHTGEKPFQCRICMRNFSDRGNLTRHLRTHLRGS | |
| CMV:ZFP | GAGGCAT | SRPGERPFQCRICMRNFSRREHLVRHLRTHTGEKPFQCRICMRNF | 2185 |
| 657- | CTGGGCG | SQNSHLRRHLKTHTGGGGSQKPFQCRICMRNFSRREHLMRHLRT | |
| OFF(mil) | GAGGG | HTGEKPFQCRICMRNFSARNTLKGHLKTHTGSQKPFQCRICMRNF | |
| | | SQSGTLHRHLRTHTGEKPFQCRICMRNFSRRDHLLRHLKTHLRGS | |
| CMV:ZFP | GGGTGTT | SRPGERPFQCRICMRNFSQQTNLTRHLRTHTGEKPFQCRICMRNF | 2186 |
| 583- | CAGAAAC | SQGGILYRHLKTHTGSQKPFQCRICMRNFSQRGNLARHLRTHTGE | |
| OFF(mil) | AGAA | KPFQCRICMRNFSTKQWTLGHLKTHTGSQKPFQCRICMRNFSRR | |
| | | QHLQYHTRTHTGEKPFQCRICMRNFSRREHLVRHLRTHLRGS | |
| CMV:ZFP | GGAGTCT | SRPGERPFQCRICMRNFSTHAHLTRHTRTHTGEKPFQCRICMRNF | 2187 |
| 638- | GAGAAAA | SRQDNLHTHLRTHTGSQKPFQCRICMRNFSQNANLKRHTRTHTG | |
| OFF(mil) | GGGA | EKPFQCRICMRNFSQREHLTTHLRTHTGSQKPFQCRICMRNFSDH | |
| | | SSLKRHLRTHTGEKPFQCRICMRNFSQTTHLSRHLKTHLRGS | |
| CMV:ZFP | GGGTGTT | SRPGERPFQCRICMRNFSQRGNLTRHLRTHTGEKPFQCRICMRNF | 2188 |
| 585- | CAGAAAC | SQSNSLKYHLKTHTGSQKPFQCRICMRNFSQRGNLARHLRTHTG | |
| OFF(mil) | AGAA | EKPFQCRICMRNFSTKQWTLGHLKTHTGSQKPFQCRICMRNFSRK | |
| | | QHLTLHTRTHTGEKPFQCRICMRNFSRREHLVRHLRTHLRGS | |
| CMV:ZFP | GCATGAG | SRPGERPFQCRICMRNFSRREVLENHLRTHTGEKPFQCRICMRNFS | 2189 |
| 639- | ACTTTGA | RRDNLLRHLKTHTGSQKPFQCRICMRNFSGGAALAVHTRTHTGE | |
| OFF(mil) | GGTG | KPFQCRICMRNFSDRSNLTRHLRTHTGSQKPFQCRICMRNFSQRE | |
| | | HLKVHLRTHTGEKPFQCRICMRNFSQGGTLRRHLKTHLRGS | |
| CMV:ZFP | GGTCAGG | SRPGERPFQCRICMRNFSQLSNLTRHTRTHTGEKPFQCRICMRNFS | 2190 |
| 591- | GGACGGG | RREHLVRHLRTHTGSQKPFQCRICMRNFSKGNDLTRHTRTHTGE | |
| OFF(mil) | GGAA | KPFQCRICMRNFSRREHLVRHLRTHTGSQKPFQCRICMRNFSKGY | |
| | | NLTVHTRTHTGEKPFQCRICMRNFSHQHRLKTHLRTHLRGS | |
| CMV:ZFP | GCATGAG | SRPGERPFQCRICMRNFSRREVLENHLRTHTGEKPFQCRICMRNFS | 2191 |
| 640- | ACTTTGA | RRDNLLRHLKTHTGSQKPFQCRICMRNFSGGAALAVHTRTHTGE | |
| OFF(mil) | GGTG | KPFQCRICMRNFSDRSNLTRHLRTHTGSQKPFQCRICMRNFSQNE | |
| | | HLKVHLRTHTGEKPFQCRICMRNFSQGGTLRRHLKTHLRGS | |
| CMV:ZFP | GGCTGGG | SRPGERPFQCRICMRNFSDSSVLRRHLRTHTGEKPFQCRICMRNFS | 2192 |
| 600- | AAGGGTG | QPHGLAHHLKTHTGSQKPFQCRICMRNFSRQEHLVRHLRTHTGE | |
| OFF(mil) | TGCC | KPFQCRICMRNFSQHPNLTRHLKTHTGSQKPFQCRICMRNFSRKE | |
| | | HLVGHLRTHTGEKPFQCRICMRNFSEKSHLTRHLKTHLRGS | |
| CMV:ZFP | GCAATTT | SRPGERPFQCRICMRNFSQSSSLVRHLRTHTGEKPFQCRICMRNFS | 2193 |
| 649- | GGTGTGG | QNSHLRRHLKTHTGSQKPFQCRICMRNFSRREVLENHLRTHTGE | |
| OFF(mil) | GAGTA | KPFQCRICMRNFSVKHGLTRHLKTHTGGGGSQKPFQCRICMRNFS | |
| | | HRESLTVHLRTHTGEKPFQCRICMRNFSQTATLKRHLKTHLRGS | |
| CMV:ZFP | TAAGTTG | SRPGERPFQCRICMRNFSRRDNLHTHLRTHTGEKPFQCRICMRNF | 2194 |
| 606- | CTACGGG | SQGGHLKRHLKTHTGSQKPFQCRICMRNFSRQVTLTRHTRTHTG | |
| OFF(mil) | AAAG | EKPFQCRICMRNFSVRETLVRHLRTHTGSQKPFQCRICMRNFSHK | |
| | | SSLTRHLRTHTGEKPFQCRICMRNFSQQGNLQLHLKTHLRGS | |
| CMV:ZFP | GCAATTT | SRPGERPFQCRICMRNFSQSSSLVRHLRTHTGEKPFQCRICMRNFS | 2195 |
| 650- | GGTGTGG | QTTHLARHLKTHTGSQKPFQCRICMRNFSRREVLENHLRTHTGE | |
| OFF(mil) | GAGTA | KPFQCRICMRNFSIRHHLKRHLKTHTGGGGSQKPFQCRICMRNFS | |
| | | HRESLTVHLRTHTGEKPFQCRICMRNFSQTATLKRHLKTHLRGS | |
| CMV:ZFP | CTGGAAT | SRPGERPFQCRICMRNFSSRFNLSTHTRTHTGEKPFQCRICMRNFS | 2196 |
| 618- | GGCAGGA | DASNLRRHLRTHTGSQKPFQCRICMRNFSRKDNLNTHLRTHTGE | |
| OFF(mil) | CCAG | KPFQCRICMRNFSRTAHLQVHLKTHTGSQKPFQCRICMRNFSQRG | |
| | | NLARHLRTHTGEKPFQCRICMRNFSRNHSLRMHLKTHLRGS | |
| CMV:ZFP | GAGGCAT | SRPGERPFQCRICMRNFSRREHLVRHLRTHTGEKPFQCRICMRNF | 2197 |
| 658- | CTGGGCG | SQNSHLRRHLKTHTGGGGSQKPFQCRICMRNFSRREHLVRHLRT | |
| OFF(mil) | GAGGG | HTGEKPFQCRICMRNFSQRNTLLNHLKTHTGSQKPFQCRICMRNF | |
| | | SQSTTLKRHLRTHTGEKPFQCRICMRNFSRQDNLQRHLKTHLRGS | |
| CMV:ZFP | CTGGAAT | SRPGERPFQCRICMRNFSKKFNLQAHTRTHTGEKPFQCRICMRNF | 2198 |
| 620- | GGCAGGA | SDPSNLARHLRTHTGSQKPFQCRICMRNFSRKDNLNTHLRTHTGE | |
| OFF(mil) | CCAG | KPFQCRICMRNFSRTAHLQVHLKTHTGSQKPFQCRICMRNFSQRS | |
| | | NLARHTRTHTGEKPFQCRICMRNFSRKESLDVHLRTHLRGS | |
| CMV:ZFP | GGAGTGG | SRPGERPFQCRICMRNFSVRSRLKLHTRTHTGEKPFQCRICMRNFS | 2199 |
| 626- | AGTCTGG | ERGHLTRHLRTHTGSQKPFQCRICMRNFSSKPNLKMHTRTHTGE | |
| OFF(mil) | CAGA | KPFQCRICMRNFSRGDNLGRHLRTHTGSQKPFQCRICMRNFSRNF | |
| | | ILQRHTRTHTGEKPFQCRICMRNFSQSAHLKRHLRTHLRGS | |
| CMV:ZFP | GTGGTAT | SRPGERPFQCRICMRNFSLKQHLVVHLRTHTGEKPFQCRICMRNF | 2200 |
| 750- | CAAGGAG | SQAGHLTRHLKTHTGSQKPFQCRICMRNFSDNAHLARHTRTHTG | |
| OFF(mil) | GAAGT | EKPFQCRICMRNFSQKCNLKAHLRTHTGGGGSQKPFQCRICMRN | |
| | | FSQSSSLVRHLRTHTGEKPFQCRICMRNFSRKERLATHLKTHLRG | |
| | | S | |
| CMV:ZFP | GTAGTCA | SRPGERPFQCRICMRNFSRATHLTRHTRTHTGEKPFQCRICMRNFS | 2201 |
| 754- | GATGGTTT | RADVLKGHLRTHTGSQKPFQCRICMRNFSLPHHLKRHLRTHTGE | |
| OFF(mil) | GGGG | KPFQCRICMRNFSISHNLARHLKTHTGGGGSQKPFQCRICMRNFS | |
| | | TRAVLRRHTRTHTGEKPFQCRICMRNFSQQSSLVRHLRTHLRGS | |
| CMV:ZFP | GAATTTG | SRPGERPFQCRICMRNFSQQTNLTRHLRTHTGEKPFQCRICMRNF | 2202 |
| 770- | GTGAAGA | SQGGILYRHLKTHTGGGGSQKPFQCRICMRNFSQAGNLSRHLRT | |
| OFF(mil) | CAGAA | HTGEKPFQCRICMRNFSIKHHLGRHLKTHTGSQKPFQCRICMRNF | |
| | | STRTELNVHTRTHTGEKPFQCRICMRNFSQAGNLRRHLRTHLRGS | |
| CMV:ZFP | GAGGCTG | SRPGERPFQCRICMRNFSRADNLGRHLRTHTGEKPFQCRICMRNF | 2203 |
| 805- | TGTGCTTC | SARNTLKGHLKTHTGGGGSQKPFQCRICMRNFSRRRNLTLHTRT | |
| OFF(mil) | TGAG | HTGEKPFQCRICMRNFSRPHVLANHLRTHTGSQKPFQCRICMRNF | |
| | | SQRSDLTRHLRTHTGEKPFQCRICMRNFSRQDNLQRHLKTHLRGS | |
| CMV:ZFP | AGGGCAC | SRPGERPFQCRICMRNFSRSESLTIHLRTHTGEKPFQCRICMRNFSR | 2204 |
| 666- | GAGGAGG | QGHLKRHLKTHTGSQKPFQCRICMRNFSRPDNLGRHLRTHTGEK | |
| OFF(mil) | GGCTG | PFQCRICMRNFSRRDNLNRHLKTHTGGGGSQKPFQCRICMRNFSQ | |
| | | GGTLSRHLRTHTGEKPFQCRICMRNFSRHYRLSTHLKTHLRGS | |
| CMV:ZFP | GGAGGGG | SRPGERPFQCRICMRNFSRQDNLGRHLRTHTGEKPFQCRICMRNF | 2205 |
| 742- | GAGAAGT | STNHWLLIHLKTHTGGGGSQKPFQCRICMRNFSQQTNLTRHLRT | |
| OFF(mil) | CAGAG | HTGEKPFQCRICMRNFSQTTHLSRHLKTHTGSQKPFQCRICMRNF | |
| | | SKKDHLHRHTRTHTGEKPFQCRICMRNFSQSAHLKRHLRTHLRG | |
| | | S | |
| CMV:ZFP | TGGGGAT | SRPGERPFQCRICMRNFSVNHNLVRHLRTHTGEKPFQCRICMRNF | 2206 |
| 675- | GGGGGTG | SRRDHLLRHLKTHTGGGGSQKPFQCRICMRNFSRKHHLGRHTRT | |
| OFF(mil) | AGGAT | HTGEKPFQCRICMRNFSRREHLTIHLRTHTGSQKPFQCRICMRNFS | |
| | | QSAHLKRHLRTHTGEKPFQCRICMRNFSRSDHLSLHLKTHLRGS | |
| CMV:ZFP | TGGGGGT | SRPGERPFQCRICMRNFSHRTNLIAHTRTHTGEKPFQCRICMRNFS | 2207 |
| 680- | GAGGATG | RREHLVRHLRTHTGSQKPFQCRICMRNFSVPHNLQRHLRTHTGE | |
| OFF(mil) | GGAAC | KPFQCRICMRNFSRVDNLGRHLKTHTGGGGSQKPFQCRICMRNF | |
| | | SRVDHLHRHLRTHTGEKPFQCRICMRNFSRSDHLSLHLKTHLRGS | |
| CMV:ZFP | GAACAGA | SRPGERPFQCRICMRNFSKHSNLTRHTRTHTGEKPFQCRICMRNFS | 2208 |
| 709- | TGGTGGG | QQTNLTRHLRTHTGGGGSQKPFQCRICMRNFSRGTVLRRHTRTH | |
| OFF(mil) | AAGAG | TGEKPFQCRICMRNFSRNDALAVHLRTHTGSQKPFQCRICMRNFS | |
| | | TTFNLRVHTRTHTGEKPFQCRICMRNFSQTQNLTRHLRTHLRGS | |
| CMV:ZFP | GAACAGA | SRPGERPFQCRICMRNFSRQANLVRHTRTHTGEKPFQCRICMRNF | 2209 |
| 712- | TGGTGGG | SQAGNLSRHLRTHTGGGGSQKPFQCRICMRNFSRTFVLKRHTRT | |
| OFF(mil) | AAGAG | HTGEKPFQCRICMRNFSRNDALAVHLRTHTGSQKPFQCRICMRNF | |
| | | STTFNLRVHTRTHTGEKPFQCRICMRNFSQTQNLTRHLRTHLRGS | |
| CMV:ZFP | GAACAGA | SRPGERPFQCRICMRNFSKHSNLTRHTRTHTGEKPFQCRICMRNFS | 2210 |
| 713- | TGGTGGG | QQTNLTRHLRTHTGGGGSQKPFQCRICMRNFSRGTVLRRHTRTH | |
| OFF(mil) | AAGAG | TGEKPFQCRICMRNFSRNDALAVHLRTHTGSQKPFQCRICMRNFS | |
| | | QRRYLVEHTRTHTGEKPFQCRICMRNFSQQTNLARHLRTHLRGS | |
| CMV:ZFP | TAGGAAC | SRPGERPFQCRICMRNFSHHTSLVRHTRTHTGEKPFQCRICMRNFS | 2211 |
| 724- | TGGAATT | QREHLKVHLRTHTGGGGSQKPFQCRICMRNFSQRSNLARHTRTH | |
| OFF(mil) | GAGCT | TGEKPFQCRICMRNFSRKESLDVHLRTHTGSQKPFQCRICMRNFS | |
| | | QQTNLTRHLRTHTGEKPFQCRICMRNFSRPESLAPHLKTHLRGS | |
| CMV:ZFP | GATGGTT | SRPGERPFQCRICMRNFSQSSSLVRHLRTHTGEKPFQCRICMRNFS | 2212 |
| 731- | AAAGGGG | RKERLATHLKTHTGSQKPFQCRICMRNFSKGDHLRRHTRTHTGE | |
| OFF(mil) | TGGTA | KPFQCRICMRNFSQRCNLLTHLRTHTGGGGSQKPFQCRICMRNFS | |
| | | LPHHLKRHLRTHTGEKPFQCRICMRNFSISHNLARHLKTHLRGS | |

### Example 15. CIITA Silencing in Frozen Cells of Different Donors

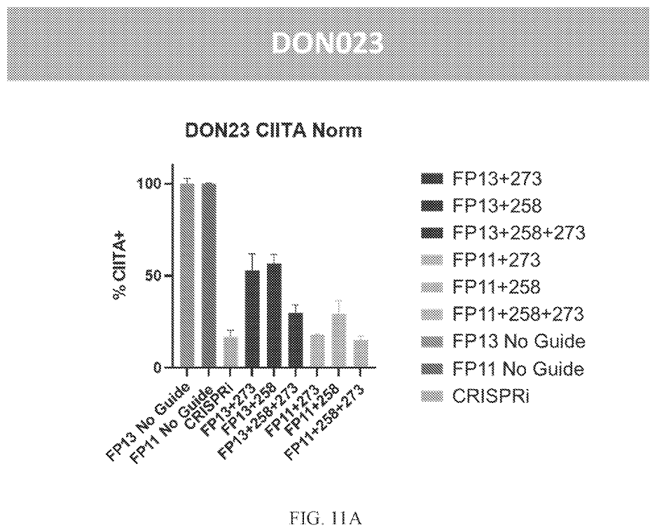

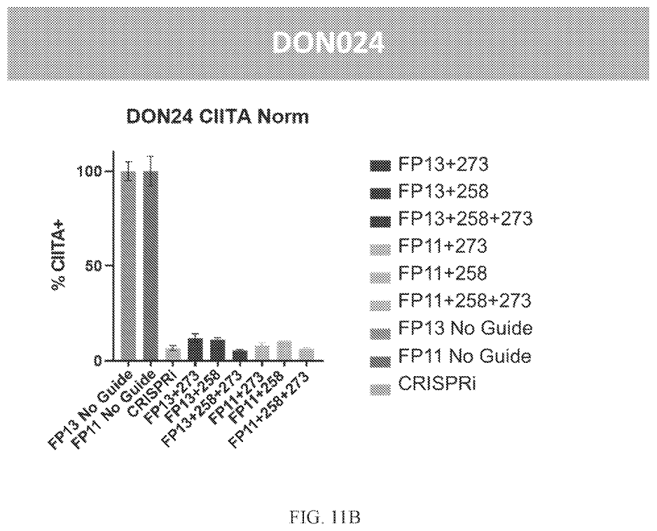

Frozen primary human T cells were transfected with various combinations of Six days after transfection, the percent of T cell expressed CIITA was measured. Silencing was achieved when effectors and guides were combined, but not when an effector or guide was used on its own. (FIGS. 11A and 11B).

### Example 16. CIITA Silencing with CRISPR-Off Variants

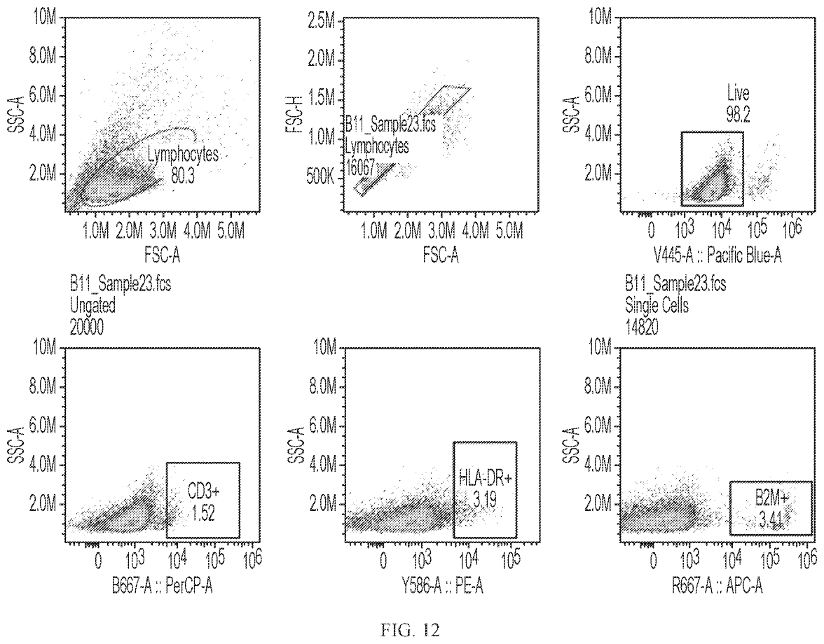

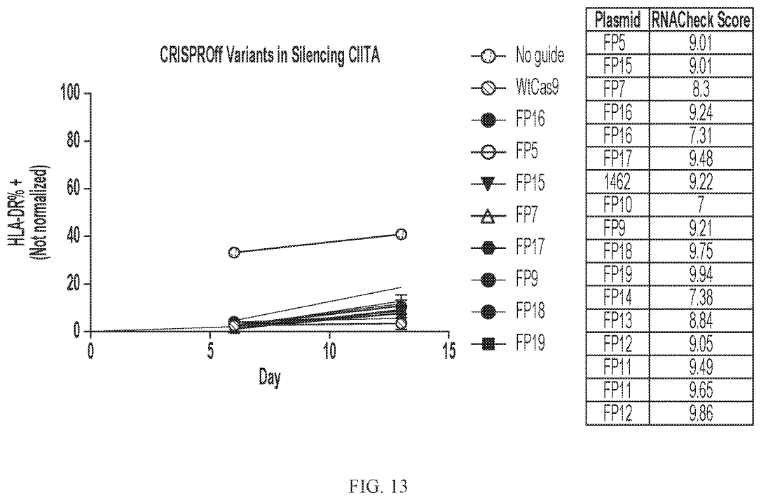

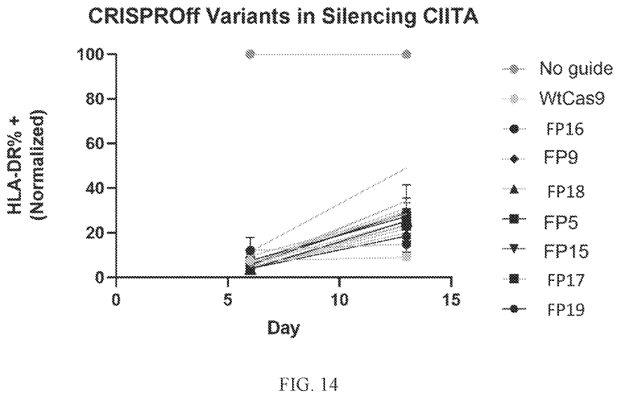

To determine the efficiency of the effectors, the top 10 plasmids were used to transfect human primary T cells from donor DON23, along with the same gRNAs in each condition. Cells were transfected with 2.5 μg of gRNAs 269 and 270 and 1 microgram of effector. Following transfection, the percent of human T cells expressing B2M, CD3, and HLA-DR was assessed. An exemplary flow cytometry gating strategy used to identify transfected cells and measure their B2M, CD3, and HLA-DR expression is shown in FIG. 12. Transfection of gRNAs with all effectors showed a reduction in HLA-DR expression (FIGS. 13-14).

### Example 17: CIITA Silencing Under Multiple Transduction Timing

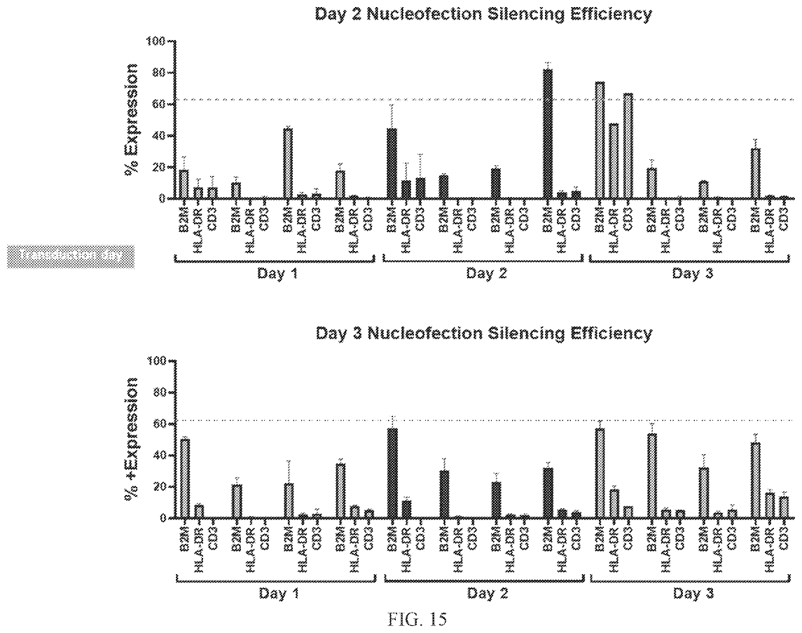

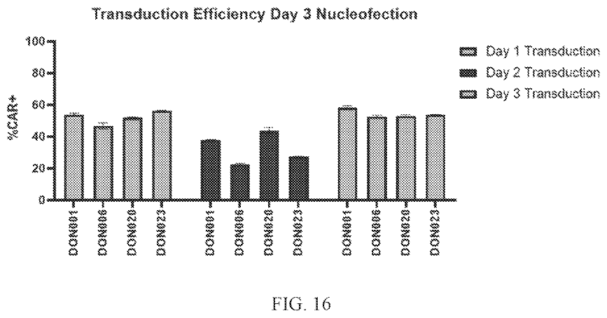

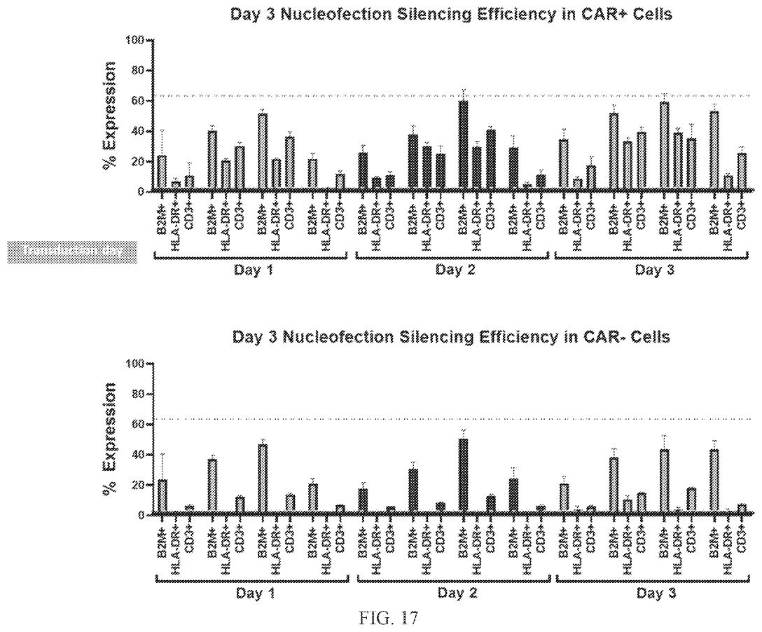

Transducing chimeric antigen receptors (CARs) into T cells that have been treated with silencing gRNAs may affect the gRNA silencing efficacy, the expression of the CAR, or both. To determine whether that was the case for the gRNAs described above, primary human T cells from donors DON001, DON006, DON020, DON023 were nucleofected at either day 2 or day 3 post-thaw. T cells were also transduced with a B-cell maturation antigen (BCMA) CAR at day 1, 2, or 3 post-thaw. T cells were transfected with pairs made from 6 different gRNAs in combination with 2.5 g of FP11a. Nucleofection with gRNAs on day 3 post-thaw resulted in more robust CIITA silencing when combined with BCMA CAR transduction, as illustrated by a reduction in B2M, HLA-DR, and CD3 expression. Additionally, B2M, HLA-DR, and CD3 expression remained lower when BCMA CAR was transduced on day 1 or day 2 post-thaw as compared to day 3. Different pairs of gRNAs exhibited varied B2M silencing ability. Results are shown in FIGS. 15-17.

### Example 18. Full Specificity Screen of Constructs in Primary Human T Cells

The specificity of CRISPR-off and ZF-off constructs for silencing CIITA was tested in primary human T cells. The readout to assess specificity was RNAseq. Genome-wide expression after epigenetic editing compared to negative controls was profiled. 6 non-limiting CIITA duplex gRNA pairs were used and compared to an effector only negative control and a WTCas9 positive control for silencing. Two replicates were performed per condition using T cells from a single donor.

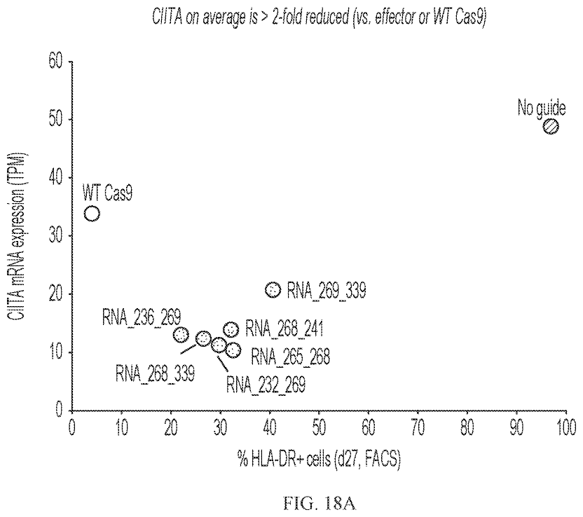

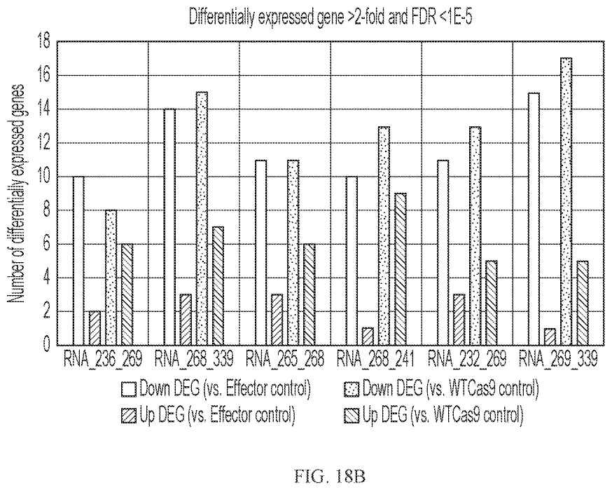

A graph of CIITA mRNA expression plotted against % HLA-DR positive cells on day 14 (as measured by FACS) is shown in FIG. 18A. On average, CIITA expression was reduced more than 2-fold after epigenetic editing compared to conditions with an effector alone or cells treated with WTCas9. FIG. 18B shows levels of genes which are differentially expressed (either upregulated (Up DEG) or downregulated (Down DEG)) after epigenetic editing compared to conditions treated with WTCas9 or an effector-only control.

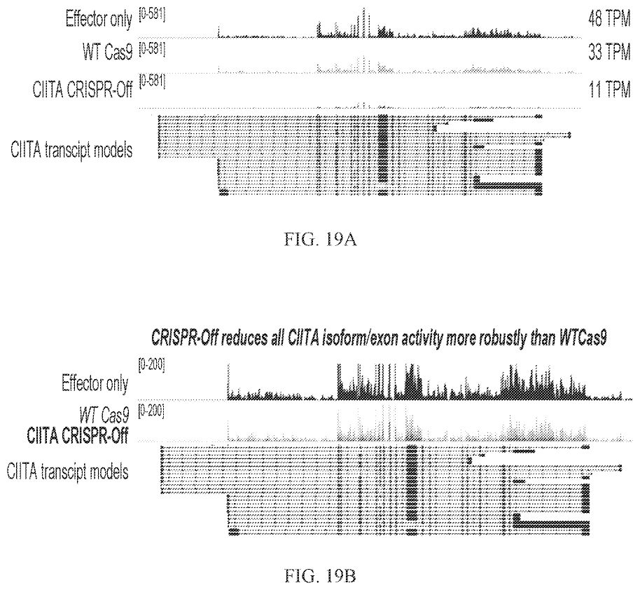

The effect of epigenetic editing, treatment with WTCas9, or an effector-only control on expression of the CIITA exon or isoform is shown in FIGS. 19A-19B. Briefly, CRISPR-Off epigenetic editing reduced CIITA isoform/exon expression more robustly than WTCas9.

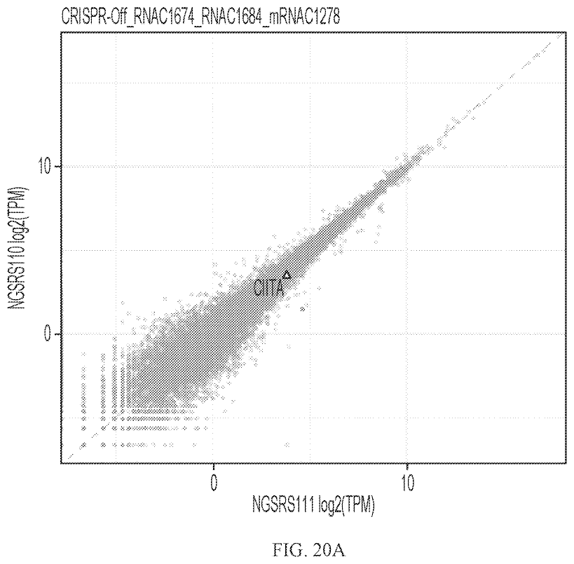

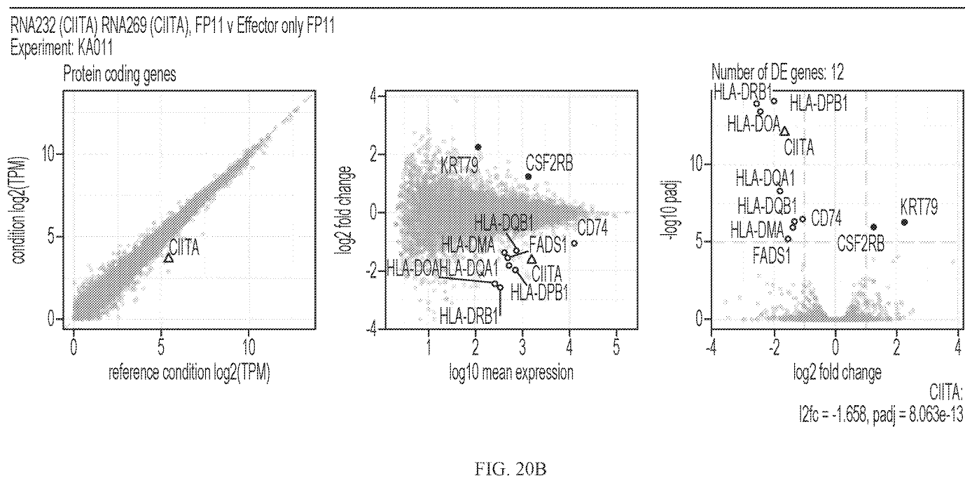

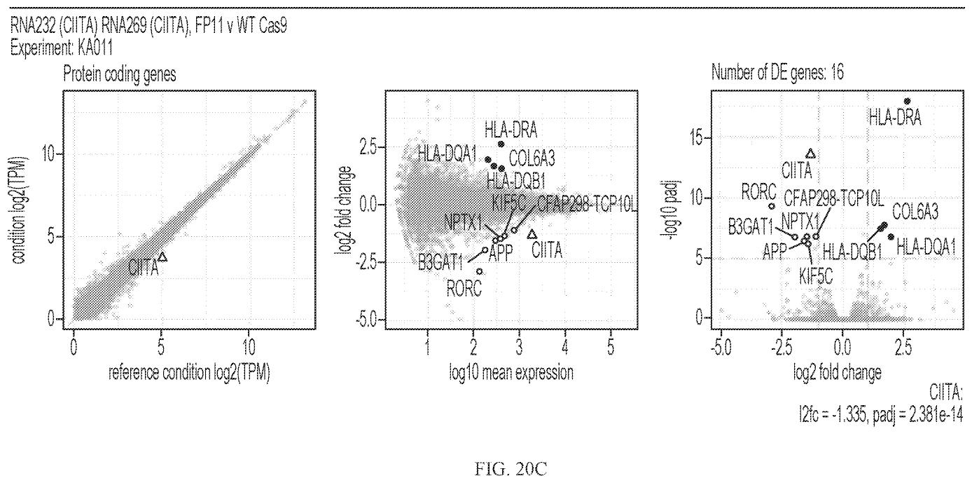

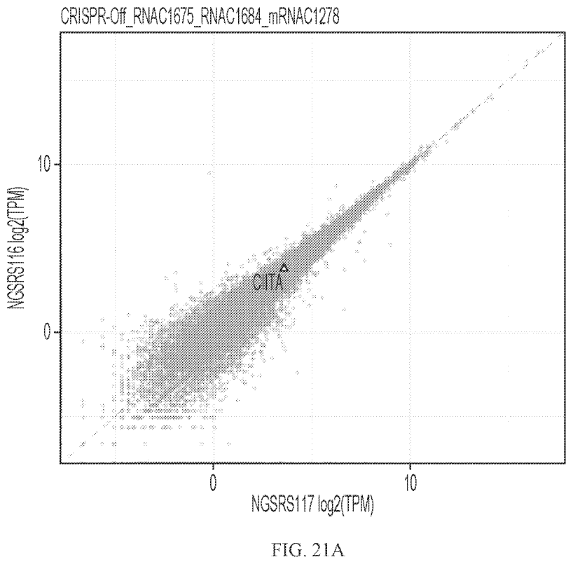

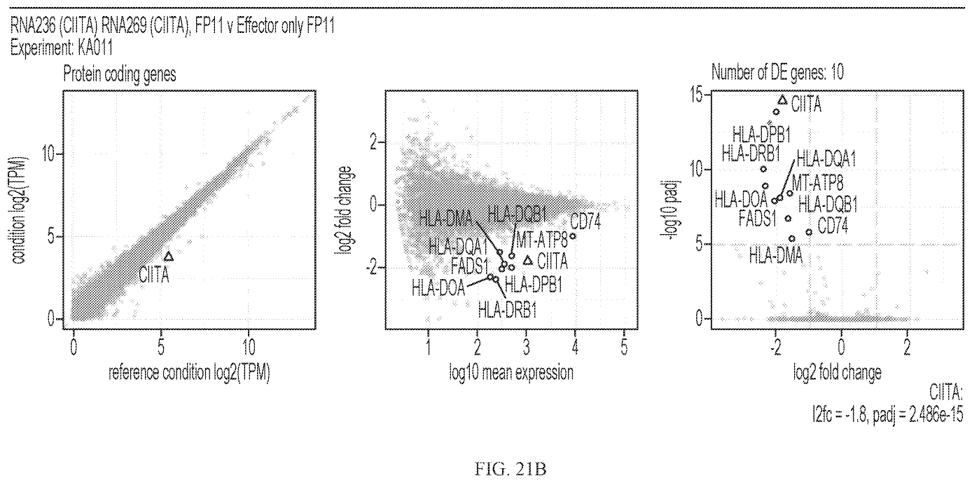

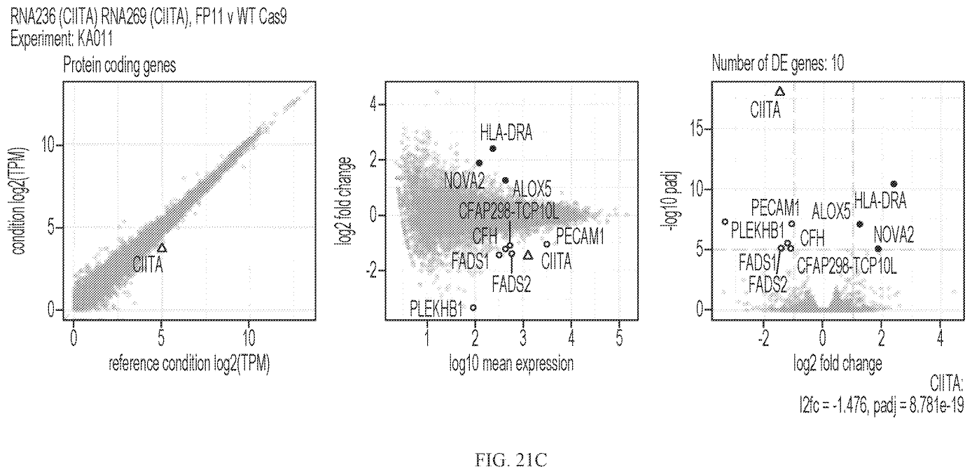

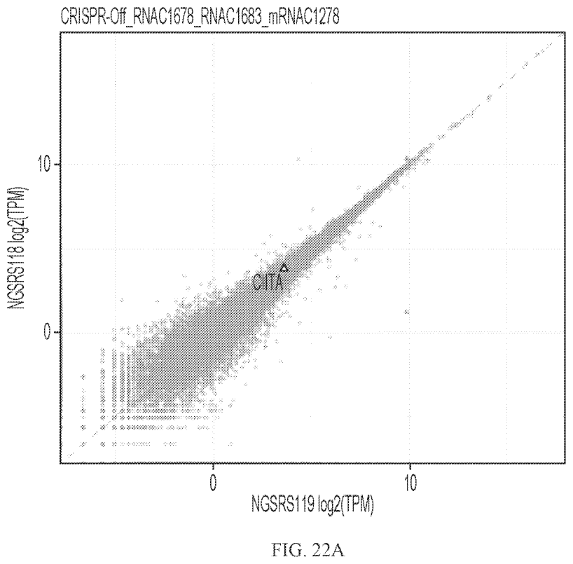

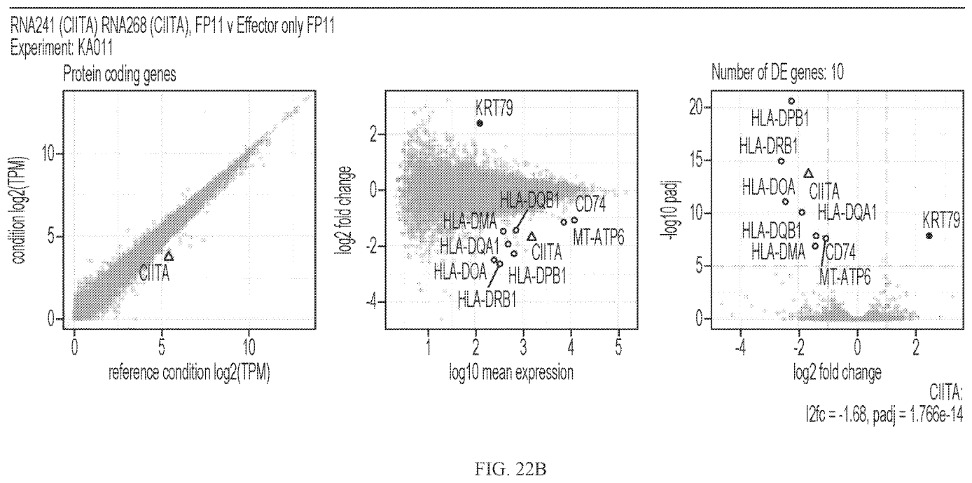

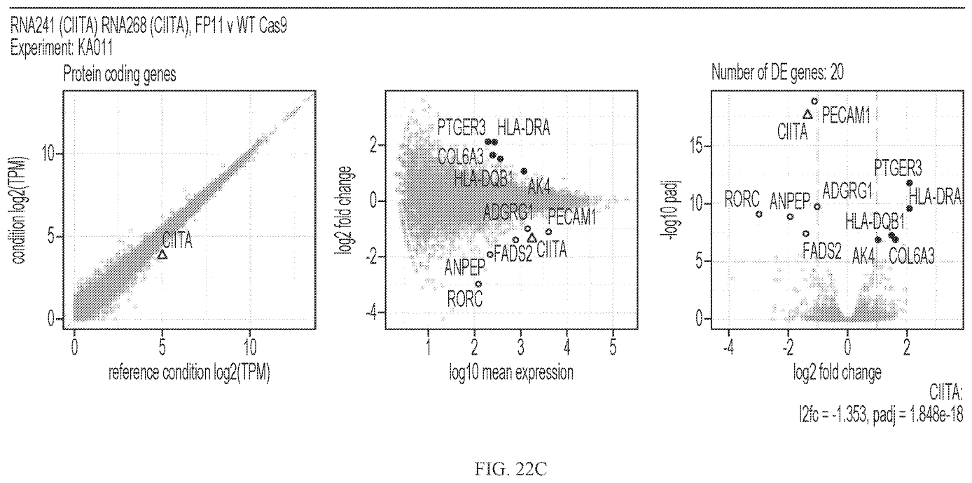

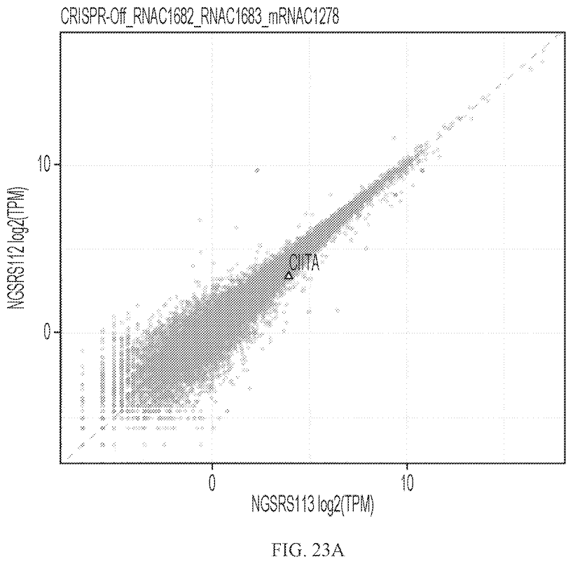

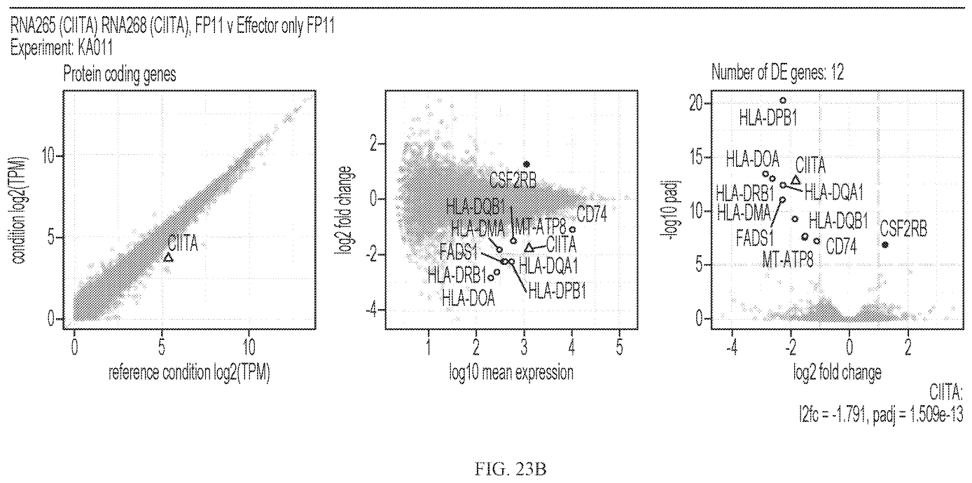

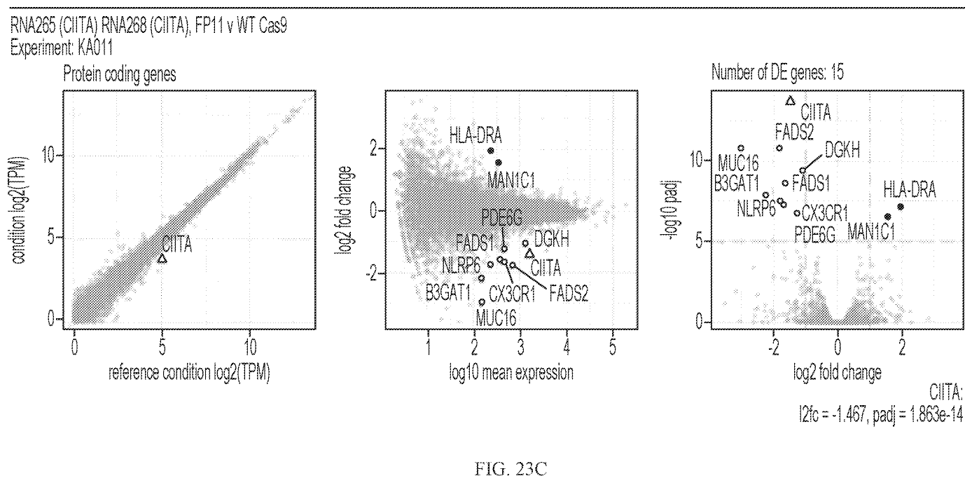

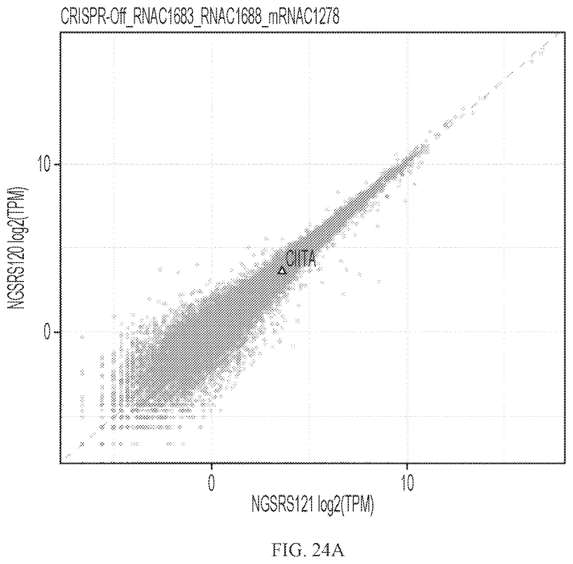

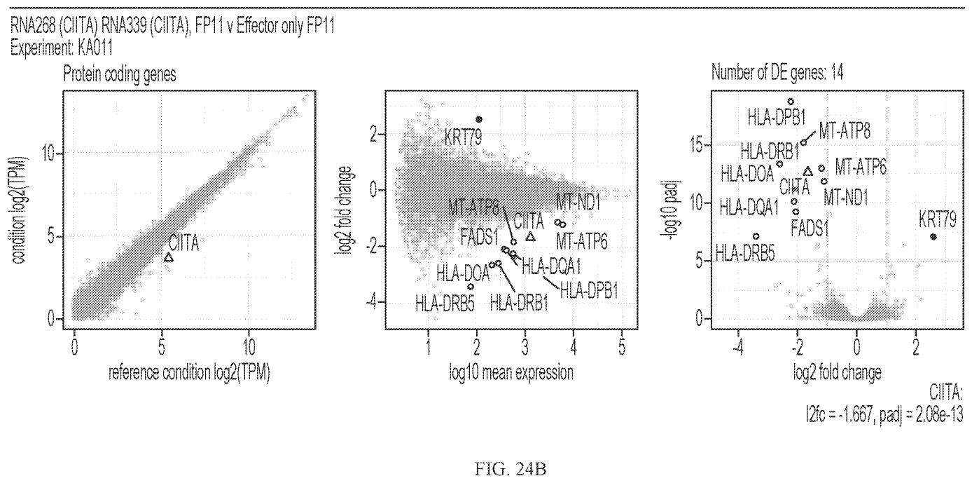

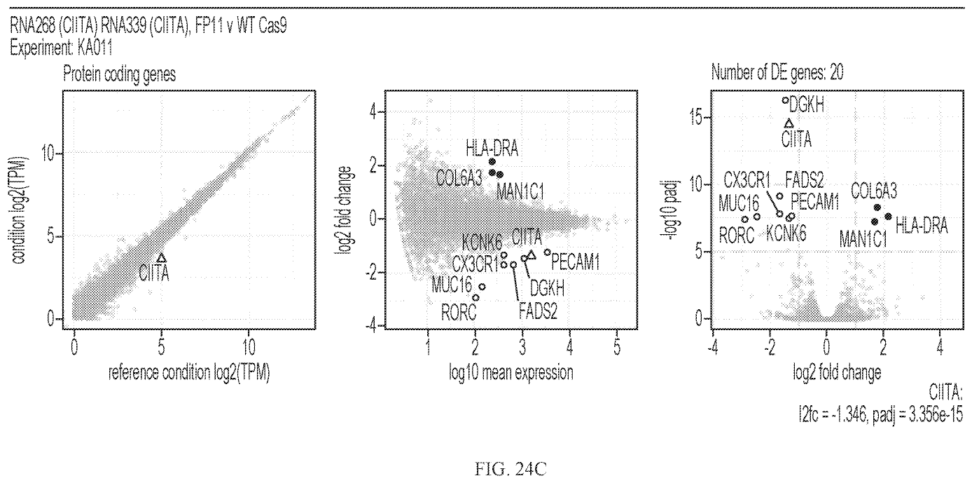

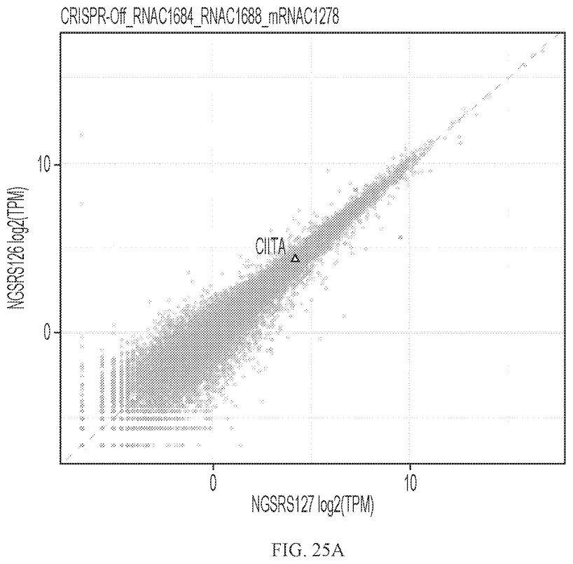

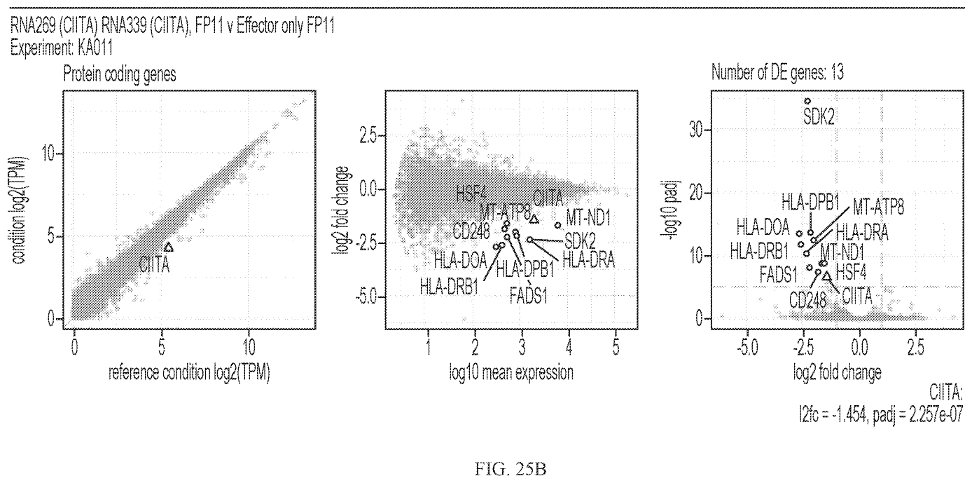

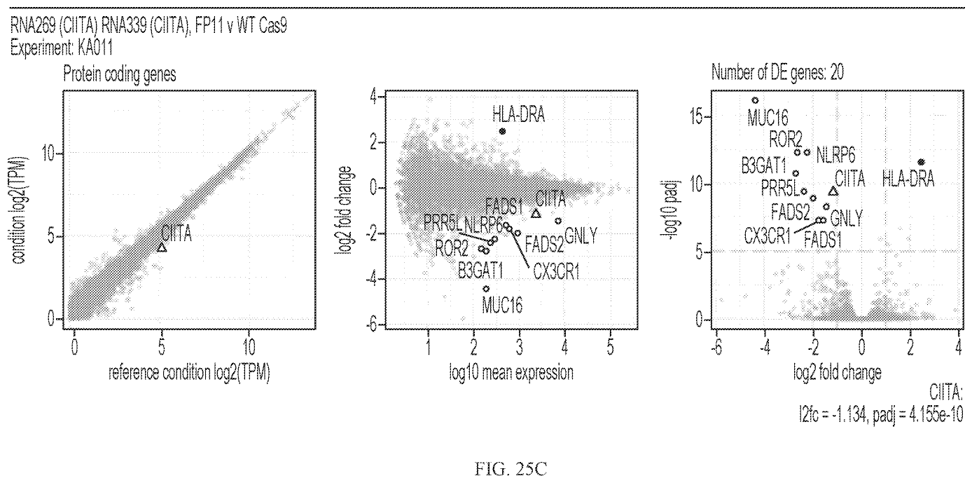

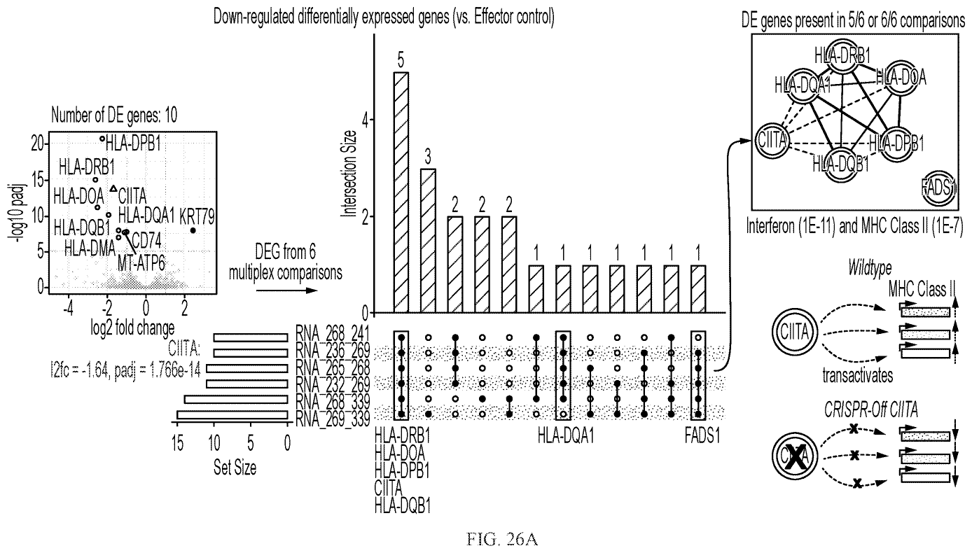

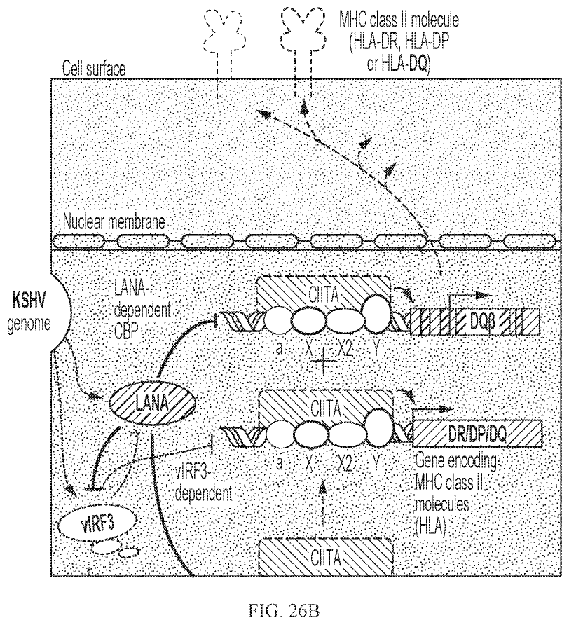

Results of an RNAseq analysis of cells treated with CRISPR-off compared to an effector only control or WTCas9 is shown in FIGS. 20A-25C. A summary of the down-regulated differentially expressed genes compared to an effector only control is shown in FIG. 26A. FIG. 26B shows an interaction map between genes differentially expressed in 5/6 or 6/6 conditions.

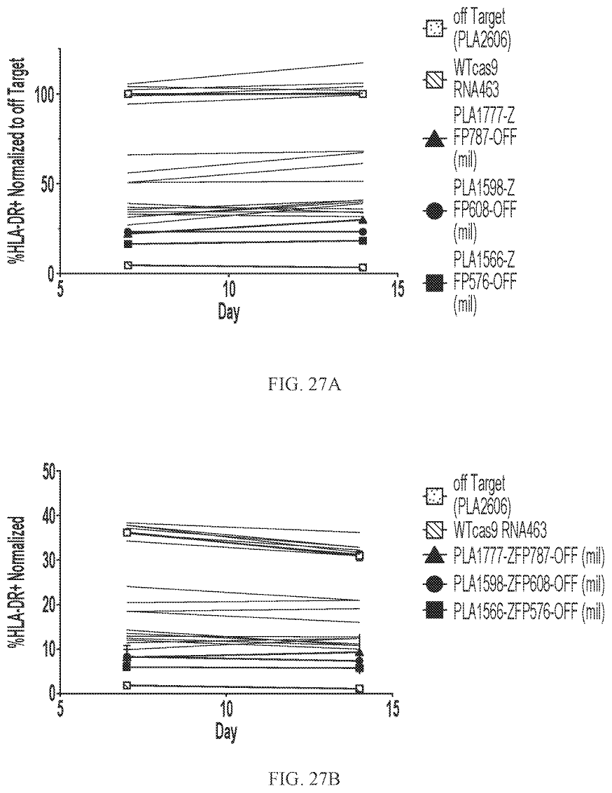

FIG. 27A-27B show ZF impact on levels of HLA-DR as normalized and normalized to off-target effects.

### Sequences

The SEQ ID NOs (SEQ) of nucleotide (nt) and amino acid (aa) sequences described in the present disclosure are listed below.

| SEQ | Description | Sequence |
|---|---|---|
| 1 | S. pyogenes WT | ATGGATAAGAAATACTCAATAGGCTTAGATATCGGCACAAATAGCG |
| | Cas9 Sequence | TCGGATGGGCGGTGATCACTGATGAATATAAGGTTCCGTCTAAAAA |
| | (nt) | GTTCAAGGTTCTGGGAAATACAGACCGCCACAGTATCAAAAAAAAT |
| | | CTTATAGGGGCTCTTTTATTTGACAGTGGAGAGACAGCGGAAGCGA |
| | | CTCGTCTCAAACGGACAGCTCGTAGAAGGTATACACGTCGGAAGAA |
| | | TCGTATTTGTTATCTACAGGAGATTTTTTCAAATGAGATGGCGAAA |
| | | GTAGATGATAGTTTCTTTCATCGACTTGAAGAGTCTTTTTTGGTGG |
| | | AAGAAGACAAGAAGCATGAACGTCATCCTATTTTTGGAAATATAGT |
| | | AGATGAAGTTGCTTATCATGAGAAATATCCAACTATCTATCATCTG |
| | | CGAAAAAAATTGGTAGATTCTACTGATAAAGCGGATTTGCGCTTAA |
| | | TCTATTTGGCCTTAGCGCATATGATTAAGTTTCGTGGTCATTTTTT |
| | | GATTGAGGGAGATTTAAATCCTGATAATAGTGATGTGGACAAACTA |
| | | TTTATCCAGTTGGTACAAACCTACAATCAATTATTTGAAGAAAACC |
| | | CTATTAACGCAAGTGGAGTAGATGCTAAAGCGATTCTTTCTGCACG |
| | | ATTGAGTAAATCAAGACGATTAGAAAATCTCATTGCTCAGCTCCCC |
| | | GGTGAGAAGAAAAATGGCTTATTTGGGAATCTCATTGCTTTGTCAT |
| | | TGGGTTTGACCCCTAATTTTAAATCAAATTTTGATTTGGCAGAAGA |
| | | TGCTAAATTACAGCTTTCAAAAGATACTTACGATGATGATTTAGAT |
| | | AATTTATTGGCGCAAATTGGAGATCAATATGCTGATTTGTTTTTGG |
| | | CAGCTAAGAATTTATCAGATGCTATTTTACTTTCAGATATCCTAAG |
| | | AGTAAATACTGAAATAACTAAGGCTCCCCTATCAGCTTCAATGATT |
| | | AAACGCTACGATGAACATCATCAAGACTTGACTCTTTTAAAAGCTT |
| | | TAGTTCGACAACAACTTCCAGAAAAGTATAAAGAAATCTTTTTTGA |
| | | TCAATCAAAAAACGGATATGCAGGTTATATTGATGGGGGAGCTAGC |
| | | CAAGAAGAATTTTATAAATTTATCAAACCAATTTTAGAAAAAATGG |
| | | ATGGTACTGAGGAATTATTGGTGAAACTAAATCGTGAAGATTTGCT |
| | | GCGCAAGCAACGGACCTTTGACAACGGCTCTATTCCCCATCAAATT |
| | | CACTTGGGTGAGCTGCATGCTATTTTGAGAAGACAAGAAGACTTTT |
| | | ATCCATTTTTAAAAGACAATCGTGAGAAGATTGAAAAAATCTTGAC |
| | | TTTTCGAATTCCTTATTATGTTGGTCCATTGGCGCGTGGCAATAGT |
| | | CGTTTTGCATGGATGACTCGGAAGTCTGAAGAAACAATTACCCCAT |
| | | GGAATTTTGAAGAAGTTGTCGATAAAGGTGCTTCAGCTCAATCATT |
| | | TATTGAACGCATGACAAACTTTGATAAAAATCTTCCAAATGAAAAA |
| | | GTACTACCAAAACATAGTTTGCTTTATGAGTATTTTACGGTTTATA |
| | | ACGAATTGACAAAGGTCAAATATGTTACTGAAGGAATGCGAAAACC |
| | | AGCATTTCTTTCAGGTGAACAGAAGAAAGCCATTGTTGATTTACTC |
| | | TTCAAAACAAATCGAAAAGTAACCGTTAAGCAATTAAAAGAAGATT |
| | | ATTTCAAAAAAATAGAATGTTTTGATAGTGTTGAAATTTCAGGAGT |
| | | TGAAGATAGATTTAATGCTTCATTAGGTACCTACCATGATTTGCTA |
| | | AAAATTATTAAAGATAAAGATTTTTTGGATAATGAAGAAAATGAAG |
| | | ATATCTTAGAGGATATTGTTTTAACATTGACCTTATTTGAAGATAG |
| | | GGAGATGATTGAGGAAAGACTTAAAACATATGCTCACCTCTTTGAT |
| | | GATAAGGTGATGAAACAGCTTAAACGTCGCCGTTATACTGGTTGGG |
| | | GACGTTTGTCTCGAAAATTGATTAATGGTATTAGGGATAAGCAATC |
| | | TGGCAAAACAATATTAGATTTTTTGAAATCAGATGGTTTTGCCAAT |
| | | CGCAATTTTATGCAGCTGATCCATGATGATAGTTTGACATTTAAAG |
| | | AAGACATTCAAAAAGCACAAGTGTCTGGACAAGGCGATAGTTTACA |
| | | TGAACATATTGCAAATTTAGCTGGTAGCCCTGCTATTAAAAAAGGT |
| | | ATTTTACAGACTGTAAAAGTTGTTGATGAATTGGTCAAAGTAATGG |
| | | GGCGGCATAAGCCAGAAAATATCGTTATTGAAATGGCACGTGAAAA |
| | | TCAGACAACTCAAAAGGGCCAGAAAAATTCGCGAGAGCGTATGAAA |
| | | CGAATCGAAGAAGGTATCAAAGAATTAGGAAGTCAGATTCTTAAAG |
| | | AGCATCCTGTTGAAAATACTCAATTGCAAAATGAAAAGCTCTATCT |
| | | CTATTATCTCCAAAATGGAAGAGACATGTATGTGGACCAAGAATTA |
| | | GATATTAATCGTTTAAGTGATTATGATGTCGATCACATTGTTCCAC |
| | | AAAGTTTCCTTAAAGACGATTCAATAGACAATAAGGTCTTAACGCG |
| | | TTCTGATAAAAATCGTGGTAAATCGGATAACGTTCCAAGTGAAGAA |
| | | GTAGTCAAAAAGATGAAAAACTATTGGAGACAACTTCTAAACGCCA |
| | | AGTTAATCACTCAACGTAAGTTTGATAATTTAACGAAAGCTGAACG |
| | | TGGAGGTTTGAGTGAACTTGATAAAGCTGGTTTTATCAAACGCCAA |
| | | TTGGTTGAAACTCGCCAAATCACTAAGCATGTGGCACAAATTTTGG |
| | | ATAGTCGCATGAATACTAAATACGATGAAAATGATAAACTTATTCG |
| | | AGAGGTTAAAGTGATTACCTTAAAATCTAAATTAGTTTCTGACTTC |
| | | CGAAAAGATTTCCAATTCTATAAAGTACGTGAGATTAACAATTACC |
| | | ATCATGCCCATGATGCGTATCTAAATGCCGTCGTTGGAACTGCTTT |
| | | GATTAAGAAATATCCAAAACTTGAATCGGAGTTTGTCTATGGTGAT |
| | | TATAAAGTTTATGATGTTCGTAAAATGATTGCTAAGTCTGAGCAAG |
| | | AAATAGGCAAAGCAACCGCAAAATATTTCTTTTACTCTAATATCAT |
| | | GAACTTCTTCAAAACAGAAATTACACTTGCAAATGGAGAGATTCGC |
| | | AAACGCCCTCTAATCGAAACTAATGGGGAAACTGGAGAAATTGTCT |
| | | GGGATAAAGGGCGAGATTTTGCCACAGTGCGCAAAGTATTGTCCAT |
| | | GCCCCAAGTCAATATTGTCAAGAAAACAGAAGTACAGACAGGCGGA |
| | | TTCTCCAAGGAGTCAATTTTACCAAAAAGAAATTCGGACAAGCTTA |
| | | TTGCTCGTAAAAAAGACTGGGATCCAAAAAAATATGGTGGTTTTGA |
| | | TAGTCCAACGGTAGCTTATTCAGTCCTAGTGGTTGCTAAGGTGGAA |
| | | AAAGGGAAATCGAAGAAGTTAAAATCCGTTAAAGAGTTACTAGGGA |
| | | TCACAATTATGGAAAGAAGTTCCTTTGAAAAAAATCCGATTGACTT |
| | | TTTAGAAGCTAAAGGATATAAGGAAGTTAAAAAAGACTTAATCATT |
| | | AAACTACCTAAATATAGTCTTTTTGAGTTAGAAAACGGTCGTAAAC |
| | | GGATGCTGGCTAGTGCCGGAGAATTACAAAAAGGAAATGAGCTGGC |
| | | TCTGCCAAGCAAATATGTGAATTTTTTATATTTAGCTAGTCATTAT |
| | | GAAAAGTTGAAGGGTAGTCCAGAAGATAACGAACAAAAACAATTGT |
| | | TTGTGGAGCAGCATAAGCATTATTTAGATGAGATTATTGAGCAAAT |
| | | CAGTGAATTTTCTAAGCGTGTTATTTTAGCAGATGCCAATTTAGAT |
| | | AAAGTTCTTAGTGCATATAACAAACATAGAGACAAACCAATACGTG |
| | | AACAAGCAGAAAATATTATTCATTTATTTACGTTGACGAATCTTGG |
| | | AGCTCCCGCTGCTTTTAAATATTTTGATACAACAATTGATCGTAAA |
| | | CGATATACGTCTACAAAAGAAGTTTTAGATGCCACTCTTATCCATC |
| | | AATCCATCACTGGTCTTTATGAAACACGCATTGATTTGAGTCAGCT |
| | | AGGAGGTGACTGA |
| 2 | S. pyogenes WT | MDKKYSIGLDIGTNSVGWAVITDEYKVPSKKFKVLGNTDRHSIKKN |
| | Cas9 Sequence | LIGALLFDSGETAEATRLKRTARRRYTRRKNRICYLQEIFSNEMAK |
| | (aa) | VDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLFD |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFDSPTVAYSVLVVAKVE |
| | | KGKSKKLKSVKELLGITIMERSSFEKNPIDFLEAKGYKEVKKDLII |
| | | KLPKYSLFELENGRKRMLASAGELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLINLGAPAAFKYFDTTIDRK |
| | | RYTSTKEVLDATLIHQSITGLYETRIDLSQLGGD |
| 3 | SaCas9 | MKRNYILGLDIGITSVGYGIIDYETRDVIDAGVRLFKEANVENNEG |
| | | RRSKRGARRLKRRRRHRIQRVKKLLFDYNLLTDHSELSGINPYEAR |
| | | VKGLSQKLSEEEFSAALLHLAKRRGVHNVNEVEEDTGNELSTKEQI |
| | | SRNSKALEEKYVAELQLERLKKDGEVRGSINRFKTSDYVKEAKQLL |
| | | KVQKAYHQLDQSFIDTYIDLLETRRTYYEGPGEGSPFGWKDIKEWY |
| | | EMLMGHCTYFPEELRSVKYAYNADLYNALNDLNNLVITRDENEKLE |
| | | YYEKFQIIENVFKQKKKPTLKQIAKEILVNEEDIKGYRVTSTGKPE |
| | | FTNLKVYHDIKDITARKEIIENAELLDQIAKILTIYQSSEDIQEEL |
| | | TNLNSELTQEEIEQISNLKGYTGTHNLSLKAINLILDELWHINDNQ |
| | | IAIFNRLKLVPKKVDLSQQKEIPTTLVDDFILSPVVKRSFIQSIKV |
| | | INAIIKKYGLPNDIIIELAREKNSKDAQKMINEMQKRNRQTNERIE |
| | | EIIRTTGKENAKYLIEKIKLHDMQEGKCLYSLEAIPLEDLLNNPFN |
| | | YEVDHIIPRSVSFDNSFNNKVLVKQEENSKKGNRTPFQYLSSSDSK |
| | | ISYETFKKHILNLAKGKGRISKTKKEYLLEERDINRFSVQKDFINR |
| | | NLVDTRYATRGLMNLLRSYFRVNNLDVKVKSINGGETSFLRRKWKF |
| | | KKERNKGYKHHAEDALIIANADFIFKEWKKLDKAKKVMENQMFEEK |
| | | QAESMPEIETEQEYKEIFITPHQIKHIKDFKDYKYSHRVDKKPNRE |
| | | LINDTLYSTRKDDKGNTLIVNNLNGLYDKDNDKLKKLINKSPEKLL |
| | | MYHHDPQTYQKLKLIMEQYGDEKNPLYKYYEETGNYLTKYSKKDNG |
| | | PVIKKIKYYGNKLNAHLDITDDYPNSRNKVVKLSLKPYRFDVYLDN |
| | | GVYKFVTVKNLDVIKKENYYEVNSKCYEEAKKLKKISNQAEFIASF |
| | | YNNDLIKINGELYRVIGVNNDLLNRIEVNMIDITYREYLENMNDKR |
| | | PPRIIKTIASKTQSIKKYSTDILGNLYEVKSKKHPQIIKKG |
| 4 | F. novicida WT | MSIYQEFVNKYSLSKTLRFELIPQGKTLENIKARGLILDDEKRAKD |
| | Cpf1 | YKKAKQIIDKYHQFFIEEILSSVCISEDLLQNYSDVYFKLKKSDDD |
| | | NLQKDFKSAKDTIKKQISEYIKDSEKFKNLFNQNLIDAKKGQESDL |
| | | ILWLKQSKDNGIELFKANSDITDIDEALEIIKSFKGWTTYFKGFHE |
| | | NRKNVYSSNDIPTSIIYRIVDDNLPKFLENKAKYESLKDKAPEAIN |
| | | YEQIKKDLAEELTFDIDYKTSEVNQRVFSLDEVFEIANFNNYLNQS |
| | | GITKFNTIIGGKFVNGENTKRKGINEYINLYSQQINDKTLKKYKMS |
| | | VLFKQILSDTESKSFVIDKLEDDSDVVTTMQSFYEQIAAFKTVEEK |
| | | SIKETLSLLFDDLKAQKLDLSKIYFKNDKSLTDLSQQVFDDYSVIG |
| | | TAVLEYITQQIAPKNLDNPSKKEQELIAKKTEKAKYLSLETIKLAL |
| | | EEFNKHRDIDKQCRFEEILANFAAIPMIFDEIAQNKDNLAQISIKY |
| | | QNQGKKDLLQASAEDDVKAIKDLLDQINNLLHKLKIFHISQSEDKA |
| | | NILDKDEHFYLVFEECYFELANIVPLYNKIRNYITQKPYSDEKFKL |
| | | NFENSTLANGWDKNKEPDNTAILFIKDDKYYLGVMNKKNNKIFDDK |
| | | AIKENKGEGYKKIVYKLLPGANKMLPKVFFSAKSIKFYNPSEDILR |
| | | IRNHSTHTKNGSPQKGYEKFEFNIEDCRKFIDFYKQSISKHPEWKD |
| | | FGFRFSDTQRYNSIDEFYREVENQGYKLTFENISESYIDSVVNQGK |
| | | LYLFQIYNKDFSAYSKGRPNLHTLYWKALFDERNLQDVVYKLNGEA |
| | | ELFYRKQSIPKKITHPAKEAIANKNKDNPKKESVFEYDLIKDKRFT |
| | | EDKFFFHCPITINFKSSGANKFNDEINLLLKEKANDVHILSIDRGE |
| | | RHLAYYTLVDGKGNIIKQDTFNIIGNDRMKTNYHDKLAAIEKDRDS |
| | | ARKDWKKINNIKEMKEGYLSQVVHEIAKLVIEYNAIVVFEDLNFGF |
| | | KRGRFKVEKQVYQKLEKMLIEKLNYLVFKDNEFDKTGGVLRAYQLT |
| | | APFETFKKMGKQTGIIYYVPAGFTSKICPVTGFVNQLYPKYESVSK |
| | | SQEFFSKFDKICYNLDKGYFEFSFDYKNFGDKAAKGKWTIASFGSR |
| | | LINFRNSDKNHNWDTREVYPTKELEKLLKDYSIEYGHGECIKAAIC |
| | | GESDKKFFAKLTSVLNTILQMRNSKTGTELDYLISPVADVNGNFFD |
| | | SRQAPKNMPQDADANGAYHIGLKGLMLLGRIKNNQEGKKLNLVIKN |
| | | EEYFEFVQNRNN |
| 5 | CasX | MEKRINKIRKKLSADNATKPVSRSGPMKTLLVRVMTDDLKKRLEKR |
| | | RKKPEVMPQVISNNAANNLRMLLDDYTKMKEAILQVYWQEFKDDHV |
| | | GLMCKFAQPASKKIDQNKLKPEMDEKGNLTTAGFACSQCGQPLFVY |
| | | KLEQVSEKGKAYTNYFGRCNVAEHEKLILLAQLKPEKDSDEAVTYS |
| | | LGKFGQRALDFYSIHVTKESTHPVKPLAQIAGNRYASGPVGKALSD |
| | | ACMGTIASFLSKYQDIIIEHQKVVKGNQKRLESLRELAGKENLEYP |
| | | SVTLPPQPHTKEGVDAYNEVIARVRMWVNLNLWQKLKLSRDDAKPL |
| | | LRLKGFPSFPVVERRENEVDWWNTINEVKKLIDAKRDMGRVFWSGV |
| | | TAEKRNTILEGYNYLPNENDHKKREGSLENPKKPAKRQFGDLLLYL |
| | | EKKYAGDWGKVFDEAWERIDKKIAGLTSHIEREEARNAEDAQSKAV |
| | | LTDWLRAKASFVLERLKEMDEKEFYACEIQLQKWYGDLRGNPFAVE |
| | | AENRVVDISGFSIGSDGHSIQYRNLLAWKYLENGKREFYLLMNYGK |
| | | KGRIRFTDGTDIKKSGKWQGLLYGGGKAKVIDLTFDPDDEQLIILP |
| | | LAFGTRQGREFIWNDLLSLETGLIKLANGRVIEKTIYNKKIGRDEP |
| | | ALFVALTFERREVVDPSNIKPVNLIGVDRGENIPAVIALTDPEGCP |
| | | LPEFKDSSGGPTDILRIGEGYKEKQRAIQAAKEVEQRRAGGYSRKF |
| | | ASKSRNLADDMVRNSARDLFYHAVTHDAVLVFENLSRGFGRQGKRT |
| | | FMTERQYTKMEDWLTAKLAYEGLTSKTYLSKTLAQYTSKTCSNCGF |
| | | TITTADYDGMLVRLKKTSDGWATTLNNKELKAEGQITYYNRYKRQT |
| | | VEKELSAELDRLSEESGNNDISKWTKGRRDEALFLLKKRFSHRPVQ |
| | | EQFVCLDCGHEVHADEQAALNIARSWLFLNSNSTEFKSYKSGKQPF |
| | | VGAWQAFYKRRLKEVWKPNA |
| 6 | CasY | MRKKLFKGYILHNKRLVYTGKAAIRSIKYPLVAPNKTALNNLSEKI |
| | | IYDYEHLFGPLNVASYARNSNRYSLVDFWIDSLRAGVIWQSKSTSL |
| | | IDLISKLEGSKSPSEKIFEQIDFELKNKLDKEQFKDIILLNTGIRS |
| | | SSNVRSLRGRFLKCFKEEFRDTEEVIACVDKWSKDLIVEGKSILVS |
| | | KQFLYWEEEFGIKIFPHFKDNHDLPKLTFFVEPSLEFSPHLPLANC |
| | | LERLKKFDISRESLLGLDNNFSAFSNYFNELFNLLSRGEIKKIVTA |
| | | VLAVSKSWENEPELEKRLHFLSEKAKLLGYPKLTSSWADYRMIIGG |
| | | KIKSWHSNYTEQLIKVREDLKKHQIALDKLQEDLKKVVDSSLREQI |
| | | EAQREALLPLLDTMLKEKDFSDDLELYRFILSDFKSLINGSYQRYI |
| | | QTEEERKEDRDVTKKYKDLYSNLRNIPRFFGESKKEQFNKFINKSL |
| | | PTIDVGLKILEDIRNALETVSVRKPPSITEEYVTKQLEKLSRKYKI |
| | | NAFNSNRFKQITEQVLRKYNNGELPKISEVFYRYPRESHVAIRILP |
| | | VKISNPRKDISYLLDKYQISPDWKNSNPGEVVDLIEIYKLTLGWLL |
| | | SCNKDFSMDFSSYDLKLFPEAASLIKNFGSCLSGYYLSKMIFNCIT |
| | | SEIKGMITLYTRDKFVVRYVTQMIGSNQKFPLLCLVGEKQTKNFSR |
| | | NWGVLIEEKGDLGEEKNQEKCLIFKDKTDFAKAKEVEIFKNNIWRI |
| | | RTSKYQIQFLNRLFKKTKEWDLMNLVLSEPSLVLEEEWGVSWDKDK |
| | | LLPLLKKEKSCEERLYYSLPLNLVPATDYKEQSAEIEQRNTYLGLD |
| | | VGEFGVAYAVVRIVRDRIELLSWGFLKDPALRKIRERVQDMKKKQV |
| | | MAVFSSSSTAVARVREMAIHSLRNQIHSIALAYKAKIIYEISISNF |
| | | ETGGNRMAKIYRSIKVSDVYRESGADTLVSEMIWGKKNKQMGNHIS |
| | | SYATSYTCCNCARTPFELVIDNDKEYEKGGDEFIFNVGDEKKVRGF |
| | | LQKSLLGKTIKGKEVLKSIKEYARPPIREVLLEGEDVEQLLKRRGN |
| | | SYIYRCPFCGYKTDADIQAALNIACRGYISDNAKDAVKEGERKLDY |
| | | ILEVRKLWEKNGAVLRSAKFL |
| 7 | CasPhi | MADTPTLFTQFLRHHLPGQRFRKDILKQAGRILANKGEDATIAFLR |
| | | GKSEESPPDFQPPVKCPIIACSRPLTEWPIYQASVAIQGYVYGQSL |
| | | AEFEASDPGCSKDGLLGWFDKTGVCTDYFSVQGLNLIFQNARKRYI |
| | | GVQTKVTNRNEKRHKKLKRINAKRIAEGLPELTSDEPESALDETGH |
| | | LIDPPGLNTNIYCYQQVSPKPLALSEVNQLPTAYAGYSTSGDDPIQ |
| | | PMVTKDRLSISKGQPGYIPEHQRALLSQKKHRRMRGYGLKARALLV |
| | | IVRIQDDWAVIDLRSLLRNAYWRRIVQTKEPSTITKLLKLVTGDPV |
| | | LDATRMVATFTYKPGIVQVRSAKCLKNKQGSKLFSERYLNETVSVT |
| | | SIDLGSNNLVAVATYRLVNGNTPELLQRFTLPSHLVKDFERYKQAH |
| | | DTLEDSIQKTAVASLPQGQQTEIRMWSMYGFREAQERVCQELGLAD |
| | | GSIPWNVMTATSTILTDLFLARGGDPKKCMFTSEPKKKKNSKQVLY |
| | | KIRDRAWAKMYRTLLSKETREAWNKALWGLKRGSPDYARLSKRKEE |
| | | LARRCVNYTISTAEKRAQCGRTIVALEDLNIGFFHGRGKQEPGWVG |
| | | LFTRKKENRWLMQALHKAFLELAHHRGYHVIEVNPAYTSQTCPVCR |
| | | HCDPDNRDQHNREAFHCIGCGFRGNADLDVATHNIAMVAITGESLK |
| | | RARGSVASKTPQPLAAE |
| 8 | Cas12f1 (Cas14a) | MIKVYRYEIVKPLDLDWKEFGTILRQLQQETRFALNKATQLAWEWM |
| | | GFSSDYKDNHGEYPKSKDILGYTNVHGYAYHTIKTKAYRLNSGNLS |
| | | QTIKRATDRFKAYQKEILRGDMSIPSYKRDIPLDLIKENISVNRMN |
| | | HGDYIASLSLLSNPAKQEMNVKRKISVIIIVRGAGKTIMDRILSGE |
| | | YQVSASQIIHDDRKNKWYLNISYDFEPQTRVLDLNKIMGIDLGVAV |
| | | AVYMAFQHTPARYKLEGGEIENFRRQVESRRISMLRQGKYAGGARG |
| | | GHGRDKRIKPIEQLRDKIANFRDTINHRYSRYIVDMAIKEGCGTIQ |
| | | MEDLINIRDIGSRFLQNWTYYDLQQKIIYKAEEAGIKVIKIDPQYT |
| | | SQRCSECGNIDSGNRIGQAIFKCRACGYEANADYNAARNIAIPNID |
| | | KIIAESIKSGGS |
| 9 | Cas12f2 (Cas14b) | NAMIAQKTIKIKLNPTKEQIIKLNSIIEEYIKVSNFTAKKIAEIQE |
| | | SFTDSGLTQGTCSECGKEKTYRKYHLLKKDNKLFCITCYKRKYSQF |
| | | TLQKVEFQNKTGLRNVAKLPKTYYTNAIRFASDTFSGFDEIIKKKQ |
| | | NRLNSIQNRLNFWKELLYNPSNRNEIKIKVVKYAPKTDTREHPHYY |
| | | SEAEIKGRIKRLEKQLKKFKMPKYPEFTSETISLQRELYSWKNPDE |
| | | LKISSITDKNESMNYYGKEYLKRYIDLINSQTPQILLEKENNSFYL |
| | | CFPITKNIEMPKIDDTFEPVGIDWGITRNIAVVSILDSKTKKPKFV |
| | | KFYSAGYILGKRKHYKSLRKHFGQKKRQDKINKLGTKEDRFIDSNI |
| | | HKLAFLIVKEIRNHSNKPIILMENITDNREEAEKSMRQNILLHSVK |
| | | SRLQNYIAYKALWNNIPTNLVKPEHTSQICNRCGHQDRENRPKGSK |
| | | LFKCVKCNYMSNADFNASINIARKFYIGEYEPFYKDNEKMKSGVNS |
| | | ISM |
| 10 | Cas12f3 (Cas14c) | MEVQKTVMKTLSLRILRPLYSQEIEKEIKEEEKERRKQAGGTGELD |
| | | GGFYKKLEKKHSEMFSFDRLNLLLNQLQREIAKVYNHAISELYIAT |
| | | IAQGNKSNKHYISSIVYNRAYGYFYNAYIALGICSKVEANFRSNEL |
| | | LTQQSALPTAKSDNFPIVLHKQKGAEGEDGGFRISTEGSDLIFEIP |
| | | IPFYEYNGENRKEPYKWVKKGGQKPVLKLILSTFRRQRNKGWAKDE |
| | | GTDAEIRKVTEGKYQVSQIEINRGKKLGEHQKWFANFSIEQPIYER |
| | | KPNRSIVGGLDVGIRSPLVCAINNSFSRYSVDSNDVFKFSKQVFAF |
| | | RRRLLSKNSLKRKHGHAAHKLEPITEMTEKNDKFRKKIIERWAKEV |
| | | TNFFVKNQVGIVQIEDLSTMKDREDHFFNQYLRGFWPYYQMQTLIE |
| | | NKLKEYGIEVKRVQAKYTSQLCSNPNCRYWNNYFNFEYRKVNKFPK |
| | | FKCEKCNLEISADYNAARNLSTPDIEKFVAKATKGINLPEK |
| 11 | C2c8 | MKVLEFKIHPTEEQVSKIDQSLAACKLLWNLSIALKEESKQRYYRK |
| | | KHKFDEFSPEIWGLSYSGHYDEKEFKTLKDKEKKLLIGNPCCKIAY |
| | | FKKTSNGKEYTPLNSIPIRRFMNAENIDKDAVNYLNRKKLAFYFRE |
| | | NTAKFIGEIETEFKKGFFKSVIKPAYDAAKKGIRGIPRFKGRRDKV |
| | | ETLVNGQPETIKIKSNGVIVSSKIGLLKIRGLDRLQGKAPRMAKIT |
| | | RKATGYYLQLTIETDDTIYKESDKCVGLDMGAVAIFTDDLGRQSEA |
| | | KRYAKIQKKRLNRLQRQASRQKDNSNNQRKTYAKLARVHEKIARQR |
| | | KGRNAQLAHKITSEYQSVILEDLNLKNMTAAAKPKEREDGDGYKQN |
| | | GKKRKSGLNKALLDNAIGQLRTFIENKANERGRKIIRVNPKHTSQT |
| | | CPNCGNIDKANRVSQSKFKCVSCGYEAHADQNAAANILIRGLRDEF |
| | | LRAIGSLYKFPVSMIGKYPGLAGEFTPDLDANQESIGDAPIENAEH |
| | | SISKQMKQEGNRTPTQPENGSQSLIFLSAPPQPCGDSHGTNNPKAL |
| | | PNKASKRSSKKPRGAIPENPDQLTIWDLLD |
| 12 | dSpCas9 | MDKKYSIGLAIGTNSVGWAVITDEYKVPSKKFKVLGNTDRHSIKKN |
| | | LIGALLFDSGETAEATRLKRTARRRYTRRKNRICYLQEIFSNEMAK |
| | | VDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMINFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLFD |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFDSPTVAYSVLVVAKVE |
| | | KGKSKKLKSVKELLGITIMERSSFEKNPIDFLEAKGYKEVKKDLII |
| | | KLPKYSLFELENGRKRMLASAGELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLINLGAPAAFKYFDTTIDRK |
| | | RYTSTKEVLDATLIHQSITGLYETRIDLSQLGGD |
| 13 | dSaCas9 | MKRNYILGLAIGITSVGYGIIDYETRDVIDAGVRLFKEANVENNEG |
| | | RRSKRGARRLKRRRRHRIQRVKKLLFDYNLLTDHSELSGINPYEAR |
| | | VKGLSQKLSEEEFSAALLHLAKRRGVHNVNEVEEDTGNELSTKEQI |
| | | SRNSKALEEKYVAELQLERLKKDGEVRGSINRFKTSDYVKEAKQLL |
| | | KVQKAYHQLDQSFIDTYIDLLETRRTYYEGPGEGSPFGWKDIKEWY |
| | | EMLMGHCTYFPEELRSVKYAYNADLYNALNDLNNLVITRDENEKLE |
| | | YYEKFQIIENVFKQKKKPTLKQIAKEILVNEEDIKGYRVTSTGKPE |
| | | FTNLKVYHDIKDITARKEIIENAELLDQIAKILTIYQSSEDIQEEL |
| | | TNLNSELTQEEIEQISNLKGYTGTHNLSLKAINLILDELWHINDNQ |
| | | IAIFNRLKLVPKKVDLSQQKEIPTTLVDDFILSPVVKRSFIQSIKV |
| | | INAIIKKYGLPNDIIIELAREKNSKDAQKMINEMQKRNRQTNERIE |
| | | EIIRTTGKENAKYLIEKIKLHDMQEGKCLYSLEAIPLEDLLNNPEN |
| | | YEVDHIIPRSVSFDNSFNNKVLVKQEEASKKGNRTPFQYLSSSDSK |
| | | ISYETFKKHILNLAKGKGRISKTKKEYLLEERDINRFSVQKDFINR |
| | | NLVDTRYATRGLMNLLRSYFRVNNLDVKVKSINGGFTSFLRRKWKF |
| | | KKERNKGYKHHAEDALIIANADFIFKEWKKLDKAKKVMENQMFEEK |
| | | QAESMPEIETEQEYKEIFITPHQIKHIKDFKDYKYSHRVDKKPNRE |
| | | LINDTLYSTRKDDKGNTLIVNNLNGLYDKDNDKLKKLINKSPEKLL |
| | | MYHHDPQTYQKLKLIMEQYGDEKNPLYKYYEETGNYLTKYSKKDNG |
| | | PVIKKIKYYGNKLNAHLDITDDYPNSRNKVVKLSLKPYRFDVYLDN |
| | | GVYKFVTVKNLDVIKKENYYEVNSKCYEEAKKLKKISNQAEFIASF |
| | | YNNDLIKINGELYRVIGVNNDLLNRIEVNMIDITYREYLENMNDKR |
| | | PPRIIKTIASKTQSIKKYSTDILGNLYEVKSKKHPQIIKKG |
| 14 | inactive FnCpf1 | MSIYQEFVNKYSLSKTLRFELIPQGKILENIKARGLILDDEKRAKD |
| | | YKKAKQIIDKYHQFFIEEILSSVCISEDLLQNYSDVYFKLKKSDDD |
| | | NLQKDFKSAKDTIKKQISEYIKDSEKFKNLFNQNLIDAKKGQESDL |
| | | ILWLKQSKDNGIELFKANSDITDIDEALEIIKSFKGWTTYFKGFHE |
| | | NRKNVYSSNDIPTSIIYRIVDDNLPKFLENKAKYESLKDKAPEAIN |
| | | YEQIKKDLAEELTFDIDYKTSEVNQRVESLDEVFEIANENNYLNQS |
| | | GITKFNTIIGGKFVNGENTKRKGINEYINLYSQQINDKTLKKYKMS |
| | | VLFKQILSDTESKSFVIDKLEDDSDVVTTMQSFYEQIAAFKTVEEK |
| | | SIKETLSLLFDDLKAQKLDLSKIYFKNDKSLTDLSQQVEDDYSVIG |
| | | TAVLEYITQQIAPKNLDNPSKKEQELIAKKTEKAKYLSLETIKLAL |
| | | EEFNKHRDIDKQCRFEEILANFAAIPMIFDEIAQNKDNLAQISIKY |
| | | QNQGKKDLLQASAEDDVKAIKDLLDQTNNLLHKLKIFHISQSEDKA |
| | | NILDKDEHFYLVFEECYFELANIVPLYNKIRNYITQKPYSDEKFKL |
| | | NFENSTLANGWDKNKEPDNTAILFIKDDKYYLGVMNKKNNKIFDDK |
| | | AIKENKGEGYKKIVYKLLPGANKMLPKVFFSAKSIKFYNPSEDILR |
| | | IRNHSTHTKNGSPQKGYEKFEFNIEDCRKFIDFYKQSISKHPEWKD |
| | | FGFRFSDTQRYNSIDEFYREVENQGYKLTFENISESYIDSVVNQGK |
| | | LYLFQIYNKDFSAYSKGRPNLHTLYWKALFDERNLQDVVYKLNGEA |
| | | ELFYRKQSIPKKITHPAKEAIANKNKDNPKKESVFEYDLIKDKRFT |
| | | EDKFFFHCPITINFKSSGANKFNDEINLLLKEKANDVHILSIARGE |
| | | RHLAYYTLVDGKGNIIKQDTFNIIGNDRMKTNYHDKLAAIEKDRDS |
| | | ARKDWKKINNIKEMKEGYLSQVVHEIAKLVIEYNAIVVFEDLNFGF |
| | | KRGRFKVEKQVYQKLEKMLIEKLNYLVFKDNEFDKTGGVLRAYQLT |
| | | APFETFKKMGKQTGIIYYVPAGFTSKICPVTGFVNQLYPKYESVSK |
| | | SQEFFSKFDKICYNLDKGYFEFSFDYKNFGDKAAKGKWTIASFGSR |
| | | LINFRNSDKNHNWDTREVYPTKELEKLLKDYSIEYGHGECIKAAIC |
| | | GESDKKFFAKLTSVLNTILQMRNSKTGTELDYLISPVADVNGNFFD |
| | | SRQAPKNMPQDADANGAYHIGLKGLMLLGRIKNNQEGKKLNLVIKN |
| | | EEYFEFVQNRNN |
| 15 | dNmeCas9 | MAAFKPNSINYILGLAIGIASVGWAMVEIDEEENPIRLIDLGVRVF |
| | | ERAEVPKTGDSLAMARRLARSVRRLTRRRAHRLLRTRRLLKREGVL |
| | | QAANFDENGLIKSLPNTPWQLRAAALDRKLTPLEWSAVLLHLIKHR |
| | | GYLSQRKNEGETADKELGALLKGVAGNAHALQTGDFRTPAELALNK |
| | | FEKESGHIRNQRSDYSHTFSRKDLQAELILLFEKQKEFGNPHVSGG |
| | | LKEGIETLLMTQRPALSGDAVQKMLGHCTFEPAEPKAAKNTYTAER |
| | | FIWLTKLNNLRILEQGSERPLTDTERATLMDEPYRKSKLTYAQARK |
| | | LLGLEDTAFFKGLRYGKDNAEASTLMEMKAYHAISRALEKEGLKDK |
| | | KSPLNLSPELQDEIGTAFSLFKTDEDITGRLKDRIQPEILEALLKH |
| | | ISFDKFVQISLKALRRIVPLMEQGKRYDEACAEIYGDHYGKKNTEE |
| | | KIYLPPIPADEIRNPVVLRALSQARKVINGVVRRYGSPARIHIETA |
| | | REVGKSFKDRKEIEKRQEENRKDREKAAAKFREYFPNFVGEPKSKD |
| | | ILKLRLYEQQHGKCLYSGKEINLGRLNEKGYVEIDAALPFSRTWDD |
| | | SFNNKVLVLGSENQNKGNQTPYEYFNGKDNSREWQEFKARVETSRF |
| | | PRSKKQRILLQKFDEDGFKERNLNDTRYVNRFLCQFVADRMRLTGK |
| | | GKKRVFASNGQITNLLRGFWGLRKVRAENDRHHALDAVVVACSTVA |
| | | MQQKITRFVRYKEMNAFDGKTIDKETGEVLHQKTHFPQPWEFFAQE |
| | | VMIRVFGKPDGKPEFEEADTLEKLRTLLAEKLSSRPEAVHEYVTPL |
| | | FVSRAPNRKMSGQGHMETVKSAKRLDEGVSVLRVPLTQLKLKDLEK |
| | | MVNREREPKLYEALKARLEAHKDDPAKAFAEPFYKYDKAGNRTQQV |
| | | KAVRVEQVQKTGVWVRNHNGIADNATMVRVDVFEKGDKYYLVPIYS |
| | | WQVAKGILPDRAVVQGKDEEDWQLIDDSFNFKFSLHPNDLVEVITK |
| | | KARMFGYFASCHRGTGNINIRIHDLDHKIGKNGILEGIGVKTALSF |
| | | QKYQIDELGKEIRPCRLKKRPPVR |
| 16 | dCjCas9 | MARILAFAIGISSIGWAFSENDELKDCGVRIFTKVENPKTGESLAL |
| | | PRRLARSARKRLARRKARLNHLKHLIANEFKLNYEDYQSFDESLAK |
| | | AYKGSLISPYELRFRALNELLSKQDFARVILHIAKRRGYDDIKNSD |
| | | DKEKGAILKAIKQNEEKLANYQSVGEYLYKEYFQKFKENSKEFTNV |
| | | RNKKESYERCIAQSFLKDELKLIFKKQREFGFSFSKKFEEEVLSVA |
| | | FYKRALKDFSHLVGNCSFFTDEKRAPKNSPLAFMFVALTRIINLLN |
| | | NLKNTEGILYTKDDLNALLNEVLKNGTLTYKQTKKLLGLSDDYEFK |
| | | GEKGTYFIEFKKYKEFIKALGEHNLSQDDLNEIAKDITLIKDEIKL |
| | | KKALAKYDLNQNQIDSLSKLEFKDHLNISFKALKLVTPLMLEGKKY |
| | | DEACNELNLKVAINEDKKDFLPAFNETYYKDEVINPVVLRAIKEYR |
| | | KVLNALLKKYGKVHKINIELAREVGKNHSQRAKIEKEQNENYKAKK |
| | | DAELECEKLGLKINSKNILKLRLFKEQKEFCAYSGEKIKISDLQDE |
| | | KMLEIDAIYPYSRSFDDSYMNKVLVFTKQNQEKLNQTPFEAFGNDS |
| | | AKWQKIEVLAKNLPTKKQKRILDKNYKDKEQKNFKDRNLNDTRYIA |
| | | RLVLNYTKDYLDFLPLSDDENTKLNDTQKGSKVHVEAKSGMLTSAL |
| | | RHTWGFSAKDRNNHLHHAIDAVIIAYANNSIVKAFSDFKKEQESNS |
| | | AELYAKKISELDYKNKRKFFEPFSGFRQKVLDKIDEIFVSKPERKK |
| | | PSGALHEETFRKEEEFYQSYGGKEGVLKALELGKIRKVNGKIVKNG |
| | | DMFRVDIFKHKKINKFYAVPIYTMDFALKVLPNKAVARSKKGEIKD |
| | | WILMDENYEFCFSLYKDSLILIQTKDMQEPEFVYYNAFTSSTVSLI |
| | | VSKHDNKFETLSKNQKILFKNANEKEVIAKSIGIQNLKVFEKYIVS |
| | | ALGEVTKAEFRQREDFKK |
| 17 | dSt1Cas9 | MGSDLVLGLAIGIGSVGVGILNKVTGEIIHKNSRIFPAAQAENNLV |
| | | RRTNRQGRRLARRKKHRRVRLNRLFEESGLITDFTKISININPYQL |
| | | RVKGLTDELSNEELFIALKNMVKHRGISYLDDASDDGNSSVGDYAQ |
| | | IVKENSKQLETKTPGQIQLERYQTYGQLRGDFTVEKDGKKHRLINV |
| | | FPTSAYRSEALRILQTQQEFNPQITDEFINRYLEILIGKRKYYHGP |
| | | GNEKSRTDYGRYRTSGETLDNIFGILIGKCTFYPDEFRAAKASYTA |
| | | QEFNLLNDLNNLTVPTETKKLSKEQKNQIINYVKNEKAMGPAKLFK |
| | | YIAKLLSCDVADIKGYRIDKSGKAEIHTFEAYRKMKTLETLDIEQM |
| | | DRETLDKLAYVLTLNTEREGIQEALEHEFADGSFSQKQVDELVQFR |
| | | KANSSIFGKGWHNFSVKLMMELIPELYETSEEQMTILTRLGKQKTT |
| | | SSSNKTKYIDEKLLTEEIYNPVVAKSVRQAIKIVNAAIKEYGDEDN |
| | | IVIEMARETNEDDEKKAIQKIQKANKDEKDAAMLKAANQYNGKAEL |
| | | PHSVFHGHKQLATKIRLWHQQGERCLYTGKTISIHDLINNSNQFEV |
| | | DAILPLSITFDDSLANKVLVYATANQEKGQRTPYQALDSMDDAWSF |
| | | RELKAFVRESKTLSNKKKEYLLTEEDISKFDVRKKFIERNLVDTRY |
| | | ASRVVLNALQEHFRAHKIDTKVSVVRGQFTSQLRRHWGIEKTRDTY |
| | | HHHAVDALIIAASSQLNLWKKQKNTLVSYSEDQLLDIETGELISDD |
| | | EYKESVFKAPYQHFVDTLKSKEFEDSILFSYQVDSKFNRKISDATI |
| | | YATRQAKVGKDKADETYVLGKIKDIYTQDGYDAFMKIYKKDKSKFL |
| | | MYRHDPQTFEKVIEPILENYPNKQINEKGKEVPCNPFLKYKEEHGY |
| | | IRKYSKKGNGPEIKSLKYYDSKLGNHIDITPKDSNNKVVLQSVSPW |
| | | RADVYFNKTTGKYEILGLKYADLQFEKGTGTYKISQEKYNDIKKKE |
| | | GVDSDSEFKFTLYKNDLLLVKDTETKEQQLFRFLSRTMPKQKHYVE |
| | | LKPYDKQKFEGGEALIKVLGNVANSGQCKKGLGKSNISIYKVRTDV |
| | | LGNQHIIKNEGDKPKLDF |
| 18 | dSt3Cas9 | MTKPYSIGLAIGTNSVGWAVITDNYKVPSKKMKVLGNTSKKYIKKN |
| | | LLGVLLFDSGITAEGRRLKRTARRRYTRRRNRILYLQEIFSTEMAT |
| | | LDDAFFQRLDDSFLVPDDKRDSKYPIFGNLVEEKVYHDEFPTIYHL |
| | | RKYLADSTKKADLRLVYLALAHMIKYRGHFLIEGEFNSKNNDIQKN |
| | | FQDFLDTYNAIFESDLSLENSKQLEEIVKDKISKLEKKDRILKLFP |
| | | GEKNSGIFSEFLKLIVGNQADFRKCFNLDEKASLHFSKESYDEDLE |
| | | TLLGYIGDDYSDVFLKAKKLYDAILLSGELTVTDNETEAPLSSAMI |
| | | KRYNEHKEDLALLKEYIRNISLKTYNEVEKDDTKNGYAGYIDGKIN |
| | | QEDFYVYLKNLLAEFEGADYFLEKIDREDFLRKQRTFDNGSIPYQI |
| | | HLQEMRAILDKQAKFYPFLAKNKERIEKILTFRIPYYVGPLARGNS |
| | | DFAWSIRKRNEKITPWNFEDVIDKESSAEAFINRMTSFDLYLPEEK |
| | | VLPKHSLLYETFNVYNELTKVRFIAESMRDYQFLDSKQKKDIVRLY |
| | | FKDKRKVTDKDIIEYLHAIYGYDGIELKGIEKQFNSSLSTYHDLLN |
| | | IINDKEFLDDSSNEAIIEEIIHTLTIFEDREMIKQRLSKFENIFDK |
| | | SVLKKLSRRHYTGWGKLSAKLINGIRDEKSGNTILDYLIDDGISNR |
| | | NFMQLIHDDALSFKKKIQKAQIIGDEDKGNIKEVVKSLPGSPAIKK |
| | | GILQSIKIVDELVKVMGGRKPESIVVEMARENQYTNQGKSNSQQRL |
| | | KRLEKSLKELGSKILKENIPAKLSKIDNNALQNDRLYLYYLQNGKD |
| | | MYTGDDLDIDRLSNYDIDHIIPQAFLKDNSIDNKVLVSSASARGKS |
| | | DDFPSLEVVKKRKTFWYQLLKSKLISQRKFDNLTKAERGGLLPEDK |
| | | AGFIQRQLVETRQITKHVARLLDEKFNNKKDENNRAVRTVKIITLK |
| | | STLVSQFRKDFELYKVREINDFHHAHDAYLNAVIASALLKKYPKLE |
| | | PEFVYGDYPKYNSFRERKSATEKVYFYSNIMNIFKKSISLADGRVI |
| | | ERPLIEVNEETGESVWNKESDLATVRRVLSYPQVNVVKKVEEQNHG |
| | | LDRGKPKGLFNANLSSKPKPNSNENLVGAKEYLDPKKYGGYAGISN |
| | | SFAVLVKGTIEKGAKKKITNVLEFQGISILDRINYRKDKLNFLLEK |
| | | GYKDIELIIELPKYSLFELSDGSRRMLASILSINNKRGEIHKGNQI |
| | | FLSQKFVKLLYHAKRISNTINENHRKYVENHKKEFEELFYYILEFN |
| | | ENYVGAKKNGKLLNSAFQSWQNHSIDELCSSFIGPTGSERKGLFEL |
| | | TSRGSAADFEFLGVKIPRYRDYTPSSLLKDATLIHQSVTGLYETRI |
| | | DLAKLGEG |
| 19 | dLbCpf1 | MSKLEKFTNCYSLSKILRFKAIPVGKTQENIDNKRLLVEDEKRAED |
| | | YKGVKKLLDRYYLSFINDVLHSIKLKNLNNYISLFRKKTRTEKENK |
| | | ELENLEINLRKEIAKAFKGNEGYKSLFKKDIIETILPEFLDDKDEI |
| | | ALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVERNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEKL |
| | | KEIIIQKVDEIYKVYGSSEKLFDADFVLEKSLKKNDAVVAIMKDLL |
| | | DSVKSFENYIKAFFGEGKETNRDESFYGDFVLAYDILLKVDHIYDA |
| | | IRNYVTQKPYSKDKFKLYFQNPQFMGGWDKDKETDYRATILRYGSK |
| | | YYLAIMDKKYAKCLQKIDKDDVNGNYEKINYKLLPGPNKMLPKVFF |
| | | SKKWMAYYNPSEDIQKIYKNGTFKKGDMENLNDCHKLIDFFKDSIS |
| | | RYPKWSNAYDFNFSETEKYKDIAGFYREVEEQGYKVSFESASKKEV |
| | | DKLVEEGKLYMFQIYNKDFSDKSHGTPNLHTMYFKLLEDENNHGQI |
| | | RLSGGAELFMRRASLKKEELVVHPANSPIANKNPDNPKKTTTLSYD |
| | | VYKDKRFSEDQYELHIPIAINKCPKNIFKINTEVRVLLKHDDNPYV |
| | | IGIARGERNLLYIVVVDGKGNIVEQYSLNEIINNENGIRIKTDYHS |
| | | LLDKKEKERFEARQNWTSIENIKELKAGYISQVVHKICELVEKYDA |
| | | VIALEDLNSGFKNSRVKVEKQVYQKFEKMLIDKLNYMVDKKSNPCA |
| | | TGGALKGYQITNKFESFKSMSTQNGFIFYIPAWLTSKIDPSTGFVN |
| | | LLKTKYTSIADSKKFISSFDRIMYVPEEDLFEFALDYKNFSRTDAD |
| | | YIKKWKLYSYGNRIRIFRNPKKNNVEDWEEVCLTSAYKELENKYGI |
| | | NYQQGDIRALLCEQSDKAFYSSFMALMSLMLQMRNSITGRTDVDEL |
| | | ISPVKNSDGIFYDSRNYEAQENAILPKNADANGAYNIARKVLWAIG |
| | | QFKKAEDEKLDKVKIAISNKEWLEYAQTSVKH |
| 20 | inactive AsCpf1 | MTQFEGFTNLYQVSKTLRFELIPQGKTLKHIQEQGFIEEDKARNDH |
| | | YKELKPIIDRIYKTYADQCLQLVQLDWENLSAAIDSYRKEKTEETR |
| | | NALIEEQATYRNAIHDYFIGRIDNLTDAINKRHAEIYKGLFKAELF |
| | | NGKVLKQLGTVTTTEHENALLRSFDKFTTYFSGFYENRKNVFSAED |
| | | ISTAIPHRIVQDNFPKFKENCHIFTRLITAVPSLREHFENVKKAIG |
| | | IFVSTSIEEVFSFPFYNQLLTQTQIDLYNQLLGGISREAGTEKIKG |
| | | LNEVLNLAIQKNDETAHIIASLPHRFIPLFKQILSDRNILSFILEE |
| | | FKSDEEVIQSFCKYKTLLRNENVLETAEALFNELNSIDLTHIFISH |
| | | KKLETISSALCDHWDTLRNALYERRISELTGKITKSAKEKVQRSLK |
| | | HEDINLQEIISAAGKELSEAFKQKTSEILSHAHAALDQPLPTTLKK |
| | | QEEKEILKSQLDSLLGLYHLLDWFAVDESNEVDPEFSARLTGIKLE |
| | | MEPSLSFYNKARNYATKKPYSVEKFKLNFQMPTLASGWDVNKEKNN |
| | | GAILFVKNGLYYLGIMPKQKGRYKALSFEPTEKTSEGFDKMYYDYF |
| | | PDAAKMIPKCSTQLKAVTAHFQTHTTPILLSNNFIEPLEITKEIYD |
| | | LNNPEKEPKKFQTAYAKKTGDQKGYREALCKWIDFTRDFLSKYTKT |
| | | TSIDLSSLRPSSQYKDLGEYYAELNPLLYHISFQRIAEKEIMDAVE |
| | | TGKLYLFQIYNKDFAKGHHGKPNLHTLYWTGLFSPENLAKTSIKLN |
| | | GQAELFYRPKSRMKRMAHRLGEKMLNKKLKDQKTPIPDTLYQELYD |
| | | YVNHRLSHDLSDEARALLPNVITKEVSHEIIKDRRFTSDKFFFHVP |
| | | ITLNYQAANSPSKFNQRVNAYLKEHPETPIIGIARGERNLIYITVI |
| | | DSTGKILEQRSLNTIQQFDYQKKLDNREKERVAARQAWSVVGTIKD |
| | | LKQGYLSQVIHEIVDLMIHYQAVVVLENLNFGFKSKRIGIAEKAVY |
| | | QQFEKMLIDKLNCLVLKDYPAEKVGGVLNPYQLTDQFTSFAKMGTQ |
| | | SGFLFYVPAPYTSKIDPLTGFVDPFVWKTIKNHESRKHFLEGEDEL |
| | | HYDVKTGDFILHFKMNRNLSFQRGLPGFMPAWDIVFEKNETQFDAK |
| | | GTPFIAGKRIVPVIENHRFTGRYRDLYPANELIALLEEKGIVERDG |
| | | SNILPKLLENDDSHAIDTMVALIRSVLQMRNSNAATGEDYINSPVR |
| | | DLNGVCFDSRFQNPEWPMDADANGAYHIALKGQLLLNHLKESKDLK |
| | | LQNGISNQDWLAYIQELRN |
| 21 | inactive enAsCpf1 | MTQFEGFTNLYQVSKTLRFELIPQGKILKHIQEQGFIEEDKARNDH |
| | | YKELKPIIDRIYKTYADQCLQLVQLDWENLSAAIDSYRKEKTEETR |
| | | NALIEEQATYRNAIHDYFIGRIDNLTDAINKRHAEIYKGLFKAELF |
| | | NGKVLKQLGTVTTTEHENALLRSFDKFTTYFSGFYRNRKNVFSAED |
| | | ISTAIPHRIVQDNFPKFKENCHIFTRLITAVPSLREHFENVKKAIG |
| | | IFVSTSIEEVFSFPFYNQLLTQTQIDLYNQLLGGISREAGTEKIKG |
| | | LNEVLNLAIQKNDETAHIIASLPHRFIPLFKQILSDRNTLSFILEE |
| | | FKSDEEVIQSFCKYKILLRNENVLETAEALFNELNSIDLTHIFISH |
| | | KKLETISSALCDHWDTLRNALYERRISELTGKITKSAKEKVQRSLK |
| | | HEDINLQEIISAAGKELSEAFKQKTSEILSHAHAALDQPLPTTLKK |
| | | QEEKEILKSQLDSLLGLYHLLDWFAVDESNEVDPEFSARLTGIKLE |
| | | MEPSLSFYNKARNYATKKPYSVEKFKLNFQMPTLARGWDVNREKNN |
| | | GAILFVKNGLYYLGIMPKQKGRYKALSFEPTEKTSEGFDKMYYDYF |
| | | PDAAKMIPKCSTQLKAVTAHFQTHTTPILLSNNFIEPLEITKEIYD |
| | | LNNPEKEPKKFQTAYAKKTGDQKGYREALCKWIDFTRDFLSKYTKT |
| | | TSIDLSSLRPSSQYKDLGEYYAELNPLLYHISFQRIAEKEIMDAVE |
| | | TGKLYLFQIYNKDFAKGHHGKPNLHTLYWTGLFSPENLAKTSIKLN |
| | | GQAELFYRPKSRMKRMAHRLGEKMLNKKLKDQKTPIPDTLYQELYD |
| | | YVNHRLSHDLSDEARALLPNVITKEVSHEIIKDRRFTSDKFFFHVP |
| | | ITLNYQAANSPSKFNQRVNAYLKEHPETPIIGIARGERNLIYITVI |
| | | DSTGKILEQRSLNTIQQFDYQKKLDNREKERVAARQAWSVVGTIKD |
| | | LKQGYLSQVIHEIVDLMIHYQAVVVLENLNFGFKSKRTGIAEKAVY |
| | | QQFEKMLIDKLNCLVLKDYPAEKVGGVLNPYQLTDQFTSFAKMGTQ |
| | | SGFLFYVPAPYTSKIDPLTGFVDPFVWKTIKNHESRKHFLEGFDFL |
| | | HYDVKTGDFILHFKMNRNLSFQRGLPGFMPAWDIVFEKNETQFDAK |
| | | GTPFIAGKRIVPVIENHRFTGRYRDLYPANELIALLEEKGIVERDG |
| | | SNILPKLLENDDSHAIDTMVALIRSVLQMRNSNAATGEDYINSPVR |
| | | DLNGVCFDSRFQNPEWPMDADANGAYHIALKGQLLLNHLKESKDLK |
| | | LQNGISNQDWLAYIQELRN |
| 22 | inactive | MTQFEGFTNLYQVSKTLRFELIPQGKTLKHIQEQGFIEEDKARNDH |
| | HFAsCpf1 | YKELKPIIDRIYKTYADQCLQLVQLDWENLSAAIDSYRKEKTEETR |
| | | NALIEEQATYRNAIHDYFIGRTDNLTDAINKRHAEIYKGLFKAELF |
| | | NGKVLKQLGTVTTTEHENALLRSFDKFTTYFSGFYRNRKNVFSAED |
| | | ISTAIPHRIVQDNFPKFKENCHIFTRLITAVPSLREHFENVKKAIG |
| | | IFVSTSIEEVFSFPFYNQLLTQTQIDLYNQLLGGISREAGTEKIKG |
| | | LNEVLALAIQKNDETAHIIASLPHRFIPLFKQILSDRNTLSFILEE |
| | | FKSDEEVIQSFCKYKTLLRNENVLETAEALFNELNSIDLTHIFISH |
| | | KKLETISSALCDHWDTLRNALYERRISELTGKITKSAKEKVQRSLK |
| | | HEDINLQEIISAAGKELSEAFKQKTSEILSHAHAALDQPLPTTLKK |
| | | QEEKEILKSQLDSLLGLYHLLDWFAVDESNEVDPEFSARLTGIKLE |
| | | MEPSLSFYNKARNYATKKPYSVEKFKLNFQMPTLARGWDVNREKNN |
| | | GAILFVKNGLYYLGIMPKQKGRYKALSFEPTEKTSEGFDKMYYDYF |
| | | PDAAKMIPKCSTQLKAVTAHFQTHTTPILLSNNFIEPLEITKEIYD |
| | | LNNPEKEPKKFQTAYAKKTGDQKGYREALCKWIDFTRDFLSKYTKT |
| | | TSIDLSSLRPSSQYKDLGEYYAELNPLLYHISFQRIAEKEIMDAVE |
| | | TGKLYLFQIYNKDFAKGHHGKPNLHTLYWTGLFSPENLAKTSIKLN |
| | | GQAELFYRPKSRMKRMAHRLGEKMLNKKLKDQKTPIPDTLYQELYD |
| | | YVNHRLSHDLSDEARALLPNVITKEVSHEIIKDRRFTSDKFFFHVP |
| | | ITLNYQAANSPSKFNQRVNAYLKEHPETPIIGIARGERNLIYITVI |
| | | DSTGKILEQRSLNTIQQFDYQKKLDNREKERVAARQAWSVVGTIKD |
| | | LKQGYLSQVIHEIVDLMIHYQAVVVLENLNFGFKSKRIGIAEKAVY |
| | | QQFEKMLIDKLNCLVLKDYPAEKVGGVLNPYQLTDQFTSFAKMGTQ |
| | | SGFLFYVPAPYTSKIDPLTGFVDPFVWKTIKNHESRKHFLEGEDEL |
| | | HYDVKTGDFILHFKMNRNLSFQRGLPGFMPAWDIVFEKNETQFDAK |
| | | GTPFIAGKRIVPVIENHRFTGRYRDLYPANELIALLEEKGIVERDG |
| | | SNILPKLLENDDSHAIDTMVALIRSVLQMRNSNAATGEDYINSPVR |
| | | DLNGVCFDSRFQNPEWPMDADANGAYHIALKGQLLLNHLKESKDLK |
| | | LQNGISNQDWLAYIQELRN |
| 23 | inactive | MTQFEGFTNLYQVSKTLRFELIPQGKTLKHIQEQGFIEEDKARNDH |
| | RVRAsCpf1 | YKELKPIIDRIYKTYADQCLQLVQLDWENLSAAIDSYRKEKTEETR |
| | | NALIEEQATYRNAIHDYFIGRIDNLTDAINKRHAEIYKGLFKAELF |
| | | NGKVLKQLGTVTTTEHENALLRSFDKFTTYFSGFYENRKNVFSAED |
| | | ISTAIPHRIVQDNFPKFKENCHIFTRLITAVPSLREHFENVKKAIG |
| | | IFVSTSIEEVFSFPFYNQLLTQTQIDLYNQLLGGISREAGTEKIKG |
| | | LNEVLNLAIQKNDETAHIIASLPHRFIPLFKQILSDRNILSFILEE |
| | | FKSDEEVIQSFCKYKILLRNENVLETAEALFNELNSIDLTHIFISH |
| | | KKLETISSALCDHWDTLRNALYERRISELTGKITKSAKEKVQRSLK |
| | | HEDINLQEIISAAGKELSEAFKQKTSEILSHAHAALDQPLPTTLKK |
| | | QEEKEILKSQLDSLLGLYHLLDWFAVDESNEVDPEFSARLTGIKLE |
| | | MEPSLSFYNKARNYATKKPYSVEKFKLNFQMPTLARGWDVNVEKNR |
| | | GAILFVKNGLYYLGIMPKQKGRYKALSFEPTEKTSEGFDKMYYDYF |
| | | PDAAKMIPKCSTQLKAVTAHFQTHTTPILLSNNFIEPLEITKEIYD |
| | | LNNPEKEPKKFQTAYAKKTGDQKGYREALCKWIDFTRDFLSKYTKT |
| | | TSIDLSSLRPSSQYKDLGEYYAELNPLLYHISFQRIAEKEIMDAVE |
| | | TGKLYLFQIYNKDFAKGHHGKPNLHTLYWTGLFSPENLAKTSIKLN |
| | | GQAELFYRPKSRMKRMAHRLGEKMLNKKLKDQKTPIPDTLYQELYD |
| | | YVNHRLSHDLSDEARALLPNVITKEVSHEIIKDRRFTSDKFFFHVP |
| | | ITLNYQAANSPSKFNQRVNAYLKEHPETPIIGIARGERNLIYITVI |
| | | DSTGKILEQRSLNTIQQFDYQKKLDNREKERVAARQAWSVVGTIKD |
| | | LKQGYLSQVIHEIVDLMIHYQAVVVLENLNFGFKSKRTGIAEKAVY |
| | | QQFEKMLIDKLNCLVLKDYPAEKVGGVLNPYQLTDQFTSFAKMGTQ |
| | | SGFLFYVPAPYTSKIDPLTGFVDPFVWKTIKNHESRKHFLEGEDEL |
| | | HYDVKTGDFILHFKMNRNLSFQRGLPGFMPAWDIVFEKNETQFDAK |
| | | GTPFIAGKRIVPVIENHRFTGRYRDLYPANELIALLEEKGIVERDG |
| | | SNILPKLLENDDSHAIDTMVALIRSVLQMRNSNAATGEDYINSPVR |
| | | DLNGVCFDSRFQNPEWPMDADANGAYHIALKGQLLLNHLKESKDLK |
| | | LQNGISNQDWLAYIQELRN |
| 24 | inactive | MTQFEGFTNLYQVSKTLRFELIPQGKTLKHIQEQGFIEEDKARNDH |
| | RRAsCpf1 | YKELKPIIDRIYKTYADQCLQLVQLDWENLSAAIDSYRKEKTEETR |
| | | NALIEEQATYRNAIHDYFIGRTDNLTDAINKRHAEIYKGLFKAELF |
| | | NGKVLKQLGTVTTTEHENALLRSFDKFTTYFSGFYENRKNVFSAED |
| | | ISTAIPHRIVQDNFPKFKENCHIFTRLITAVPSLREHFENVKKAIG |
| | | IFVSTSIEEVFSFPFYNQLLTQTQIDLYNQLLGGISREAGTEKIKG |
| | | LNEVLNLAIQKNDETAHIIASLPHRFIPLFKQILSDRNTLSFILEE |
| | | FKSDEEVIQSFCKYKTLLRNENVLETAEALFNELNSIDLTHIFISH |
| | | KKLETISSALCDHWDTLRNALYERRISELTGKITKSAKEKVQRSLK |
| | | HEDINLQEIISAAGKELSEAFKQKTSEILSHAHAALDQPLPTTLKK |
| | | QEEKEILKSQLDSLLGLYHLLDWFAVDESNEVDPEFSARLIGIKLE |
| | | MEPSLSFYNKARNYATKKPYSVEKFKLNFQMPTLARGWDVNKEKNN |
| | | GAILFVKNGLYYLGIMPKQKGRYKALSFEPTEKTSEGFDKMYYDYF |
| | | PDAAKMIPRCSTQLKAVTAHFQTHTTPILLSNNFIEPLEITKEIYD |
| | | LNNPEKEPKKFQTAYAKKTGDQKGYREALCKWIDFTRDFLSKYTKT |
| | | TSIDLSSLRPSSQYKDLGEYYAELNPLLYHISFQRIAEKEIMDAVE |
| | | TGKLYLFQIYNKDFAKGHHGKPNLHTLYWTGLFSPENLAKTSIKLN |
| | | GQAELFYRPKSRMKRMAHRLGEKMLNKKLKDQKTPIPDTLYQELYD |
| | | YVNHRLSHDLSDEARALLPNVITKEVSHEIIKDRRFTSDKFFFHVP |
| | | ITLNYQAANSPSKFNQRVNAYLKEHPETPIIGIARGERNLIYITVI |
| | | DSTGKILEQRSLNTIQQFDYQKKLDNREKERVAARQAWSVVGTIKD |
| | | LKQGYLSQVIHEIVDLMIHYQAVVVLENLNFGFKSKRTGIAEKAVY |
| | | QQFEKMLIDKLNCLVLKDYPAEKVGGVLNPYQLTDQFTSFAKMGTQ |
| | | SGFLFYVPAPYTSKIDPLTGFVDPFVWKTIKNHESRKHFLEGEDEL |
| | | HYDVKTGDFILHFKMNRNLSFQRGLPGFMPAWDIVFEKNETQFDAK |
| | | GTPFIAGKRIVPVIENHRFTGRYRDLYPANELIALLEEKGIVERDG |
| | | SNILPKLLENDDSHAIDTMVALIRSVLQMRNSNAATGEDYINSPVR |
| | | DLNGVCFDSRFQNPEWPMDADANGAYHIALKGQLLLNHLKESKDLK |
| | | LQNGISNQDWLAYIQELRN |
| 25 | dCasX | MEKRINKIRKKLSADNATKPVSRSGPMKILLVRVMTDDLKKRLEKR |
| | | RKKPEVMPQVISNNAANNLRMLLDDYTKMKEAILQVYWQEFKDDHV |
| | | GLMCKFAQPASKKIDQNKLKPEMDEKGNLTTAGFACSQCGQPLEVY |
| | | KLEQVSEKGKAYTNYFGRCNVAEHEKLILLAQLKPEKDSDEAVTYS |
| | | LGKFGQRALDFYSIHVTKESTHPVKPLAQIAGNRYASGPVGKALSD |
| | | ACMGTIASFLSKYQDIIIEHQKVVKGNQKRLESLRELAGKENLEYP |
| | | SVTLPPQPHTKEGVDAYNEVIARVRMWVNLNLWQKLKLSRDDAKPL |
| | | LRLKGFPSFPVVERRENEVDWWNTINEVKKLIDAKRDMGRVFWSGV |
| | | TAEKRNTILEGYNYLPNENDHKKREGSLENPKKPAKRQFGDLLLYL |
| | | EKKYAGDWGKVFDEAWERIDKKIAGLTSHIEREEARNAEDAQSKAV |
| | | LTDWLRAKASFVLERLKEMDEKEFYACEIQLQKWYGDLRGNPFAVE |
| | | AENRVVDISGFSIGSDGHSIQYRNLLAWKYLENGKREFYLLMNYGK |
| | | KGRIRFTDGTDIKKSGKWQGLLYGGGKAKVIDLTFDPDDEQLIILP |
| | | LAFGTRQGREFIWNDLLSLETGLIKLANGRVIEKTIYNKKIGRDEP |
| | | ALFVALTFERREVVDPSNIKPVNLIGVARGENIPAVIALTDPEGCP |
| | | LPEFKDSSGGPTDILRIGEGYKEKQRAIQAAKEVEQRRAGGYSRKF |
| | | ASKSRNLADDMVRNSARDLFYHAVTHDAVLVFANLSRGFGRQGKRT |
| | | FMTERQYTKMEDWLTAKLAYEGLTSKTYLSKTLAQYTSKTCSNCGF |
| | | TITTADYDGMLVRLKKTSDGWATTLNNKELKAEGQITYYNRYKRQT |
| | | VEKELSAELDRLSEESGNNDISKWTKGRRDEALFLLKKRFSHRPVQ |
| | | EQFVCLDCGHEVHAAEQAALNIARSWLFLNSNSTEFKSYKSGKQPF |
| | | VGAWQAFYKRRLKEVWKPNA |
| 26 | dCasPhi | MPKPAVESEFSKVLKKHFPGERFRSSYMKRGGKILAAQGEEAVVAY |
| | | LQGKSEEEPPNFQPPAKCHVVTKSRDFAEWPIMKASEAIQRYIYAL |
| | | STTERAACKPGKSSESHAAWFAATGVSNHGYSHVQGLNLIFDHTLG |
| | | RYDGVLKKVQLRNEKARARLESINASRADEGLPEIKAEEEEVATNE |
| | | TGHLLQPPGINPSFYVYQTISPQAYRPRDEIVLPPEYAGYVRDPNA |
| | | PIPLGVVRNRCDIQKGCPGYIPEWQREAGTAISPKTGKAVTVPGLS |
| | | PKKNKRMRRYWRSEKEKAQDALLVTVRIGTDWVVIDVRGLLRNARW |
| | | RTIAPKDISLNALLDLFTGDPVIDVRRNIVTFTYTLDACGTYARKW |
| | | TLKGKQTKATLDKLTATQTVALVAIALGQTNPISAGISRVTQENGA |
| | | LQCEPLDRFTLPDDLLKDISAYRIAWDRNEEELRARSVEALPEAQQ |
| | | AEVRALDGVSKETARTQLCADFGLDPKRLPWDKMSSNITFISEALL |
| | | SNSVSRDQVFFTPAPKKGAKKKAPVEVMRKDRTWARAYKPRLSVEA |
| | | QKLKNEALWALKRTSPEYLKLSRRKEELCRRSINYVIEKTRRRTQC |
| | | QIVIPVIEDLNVRFFHGSGKRLPGWDNFFTAKKENRWFIQGLHKAF |
| | | SDLRTHRSFYVFEVRPERTSITCPKCGHCEVGNRDGEAFQCLSCGK |
| | | TCNADLDVATHNLTQVALTGKTMPKREEPRDAQGTAPARKTKKASK |
| | | SKAPPAEREDQTPAQEPSQTS |
| 27 | inactive VRER | MDKKYSIGLAIGTNSVGWAVITDEYKVPSKKFKVLGNTDRHSIKKN |
| | SpCas9 | LIGALLFDSGETAEATRLKRTARRRYTRRKNRICYLQEIFSNEMAK |
| | | VDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNEDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLED |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFVSPTVAYSVLVVAKVE |
| | | KGKSKKLKSVKELLGITIMERSSFEKNPIDFLEAKGYKEVKKDLII |
| | | KLPKYSLFELENGRKRMLASARELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLTNLGAPAAFKYFDTTIDRK |
| | | EYRSTKEVLDATLIHQSITGLYETRIDLSQLGGD |
| 28 | inactive EQR | MDKKYSIGLAIGTNSVGWAVITDEYKVPSKKFKVLGNTDRHSIKKN |
| | SpCas9 | LIGALLFDSGETAEATRLKRTARRRYTRRKNRICYLQEIFSNEMAK |
| | | VDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMINEDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLILTLFEDREMIEERLKTYAHLED |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDAIVPQSFLKDDSIDNKVLIRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFESPTVAYSVLVVAKVE |
| | | KGKSKKLKSVKELLGITIMERSSFEKNPIDFLEAKGYKEVKKDLII |
| | | KLPKYSLFELENGRKRMLASAGELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLTNLGAPAAFKYFDTTIDRK |
| | | QYRSTKEVLDATLIHQSITGLYETRIDLSQLGGD |
| 29 | inactive VQR | MDKKYSIGLAIGTNSVGWAVITDEYKVPSKKFKVLGNTDRHSIKKN |
| | SpCas9 | LIGALLFDSGETAEATRLKRTARRRYTRRKNRICYLQEIFSNEMAK |
| | | VDDSFFHRLEESELVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLED |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFVSPTVAYSVLVVAKVE |
| | | KGKSKKLKSVKELLGITIMERSSFEKNPIDFLEAKGYKEVKKDLII |
| | | KLPKYSLFELENGRKRMLASAGELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLTNLGAPAAFKYFDTTIDRK |
| | | QYRSTKEVLDATLIHQSITGLYETRIDLSQLGGD |
| 30 | inactive SPG | MDKKYSIGLAIGTNSVGWAVITDEYKVPSKKFKVLGNTDRHSIKKN |
| | SpCas9 | LIGALLFDSGETAEATRLKRTARRRYTRRKNRICYLQEIFSNEMAK |
| | | VDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLILFEDREMIEERLKTYAHLED |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFLWPTVAYSVLVVAKVE |
| | | KGKSKKLKSVKELLGITIMERSSFEKNPIDFLEAKGYKEVKKDLII |
| | | KLPKYSLFELENGRKRMLASAKQLQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLINLGAPAAFKYFDTTIDRK |
| | | QYRSTKEVLDATLIHQSITGLYETRIDLSQLGGD |
| 31 | inactive SpRY | MDKKYSIGLAIGTNSVGWAVITDEYKVPSKKFKVLGNTDRHSIKKN |
| | Cas9 | LIGALLFDSGETAERTRLKRTARRRYTRRKNRICYLQEIFSNEMAK |
| | | VDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLED |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDAIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESIRPKRNSDKLIARKKDWDPKKYGGFLWPTVAYSVLVVAKVE |
| | | KGKSKKLKSVKELLGITIMERSSFEKNPIDFLEAKGYKEVKKDLII |
| | | KLPKYSLFELENGRKRMLASAKQLQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLTRLGAPRAFKYFDTTIDPK |
| | | QYRSTKEVLDATLIHQSITGLYETRIDLSQLGGD |
| 32 | inactive KKH | MKRNYILGLAIGITSVGYGIIDYETRDVIDAGVRLFKEANVENNEG |
| | dSaCas9 | RRSKRGARRLKRRRRHRIQRVKKLLFDYNLLTDHSELSGINPYEAR |
| | | VKGLSQKLSEEEFSAALLHLAKRRGVHNVNEVEEDTGNELSTKEQI |
| | | SRNSKALEEKYVAELQLERLKKDGEVRGSINRFKTSDYVKEAKQLL |
| | | KVQKAYHQLDQSFIDTYIDLLETRRTYYEGPGEGSPFGWKDIKEWY |
| | | EMLMGHCTYFPEELRSVKYAYNADLYNALNDLNNLVITRDENEKLE |
| | | YYEKFQIIENVFKQKKKPTLKQIAKEILVNEEDIKGYRVTSTGKPE |
| | | FTNLKVYHDIKDITARKEIIENAELLDQIAKILTIYQSSEDIQEEL |
| | | TNLNSELTQEEIEQISNLKGYTGTHNLSLKAINLILDELWHINDNQ |
| | | IAIFNRLKLVPKKVDLSQQKEIPTTLVDDFILSPVVKRSFIQSIKV |
| | | INAIIKKYGLPNDIIIELAREKNSKDAQKMINEMQKRNRQTNERIE |
| | | EIIRTTGKENAKYLIEKIKLHDMQEGKCLYSLEAIPLEDLLNNPEN |
| | | YEVDHIIPRSVSFDNSFNNKVLVKQEEASKKGNRTPFQYLSSSDSK |
| | | ISYETFKKHILNLAKGKGRISKTKKEYLLEERDINRFSVQKDFINR |
| | | NLVDTRYATRGLMNLLRSYFRVNNLDVKVKSINGGFTSFLRRKWKF |
| | | KKERNKGYKHHAEDALIIANADFIFKEWKKLDKAKKVMENQMFEEK |
| | | QAESMPEIETEQEYKEIFITPHQIKHIKDFKDYKYSHRVDKKPNRK |
| | | LINDTLYSTRKDDKGNTLIVNNLNGLYDKDNDKLKKLINKSPEKLL |
| | | MYHHDPQTYQKLKLIMEQYGDEKNPLYKYYEETGNYLTKYSKKDNG |
| | | PVIKKIKYYGNKLNAHLDITDDYPNSRNKVVKLSLKPYRFDVYLDN |
| | | GVYKFVTVKNLDVIKKENYYEVNSKCYEEAKKLKKISNQAEFIASF |
| | | YKNDLIKINGELYRVIGVNNDLLNRIEVNMIDITYREYLENMNDKR |
| | | PPHIIKTIASKTQSIKKYSTDILGNLYEVKSKKHPQIIKKG |
| 33 | ZIM3 | MNNSQGRVTFEDVTVNFTQGEWQRLNPEQRNLYRDVMLENYSNLVS |
| | | VGQGETTKPDVILRLEQGKEPWLEEEEVLGSGRAEKNGDIGGQIWK |
| | | PKDVKESL |
| 34 | ZNF436 | MAATLLMAGSQAPVTFEDMAMYLTREEWRPLDAAQRDLYRDVMQEN |
| | | YGNVVSLDFEIRSENEVNPKQEISEDVQFGTTSERPAENAEENPES |
| | | EEGFESGDRSERQW |
| 35 | ZNF257 | MLENYRNLVFLGIAVSKPDLITCLEQGKEPCNMKRHEMVAKPPVMC |
| | | SHIAEDLCPERDIKYFFQKVILRRYDKCEHENLQLRKGCKSVDECK |
| | | VCK |
| 36 | ZNF675 | MGLLTFRDVAIEFSLEEWQCLDTAQRNLYKNVILENYRNLVFLGIA |
| | | VSKQDLITCLEQEKEPLTVKRHEMVNEPPVMCSHFAQEFWPEQNIK |
| | | DSF |
| 37 | ZNF490 | MLQMQNSEHHGQSIKTQTDSISLEDVAVNFTLEEWALLDPGQRNIY |
| | | RDVMRATFKNLACIGEKWKDQDIEDEHKNQGRNLRSPMVEALCENK |
| | | EDCPCGKSTSQIPDLNINLETPTG |
| 38 | ZNF320 | MALSQGLLTFRDVAIEFSQEEWKCLDPAQRTLYRDVMLENYRNLVS |
| | | LDISSKCMMNTLSSTGQGNTEVIHTGTLQRQASYHIGAFCSQEIEK |
| | | DIHDFVFQ |
| 39 | ZNF331 | MAQGLVTFADVAIDFSQEEWACLNSAQRDLYWDVMLENYSNLVSLD |
| | | LESAYENKSLPTKKNIHEIRASKRNSDRRSKSLGRNWICEGTLERP |
| | | QRSRGR |
| 40 | ZNF816 | MLREEATKKSKEKEPGMALPQGRLTFRDVAIEFSLEEWKCLNPAQR |
| | | ALYRAVMLENYRNLEFVDSSLKSMMEFSSTRHSITGEVIHTGTLQR |
| | | HKSHHIGDFCFPEMKKDIHHFEFQWQ |
| 41 | ZNF680 | MPGPPGSLEMGPLTFRDVAIEFSLEEWQCLDTAQRNLYRKVMFENY |
| | | RNLVFLGIAVSKPHLITCLEQGKEPWNRKRQEMVAKPPVIYSHFTE |
| | | DLWPEHSIKDSF |
| 42 | ZNF41 | MSPPWSPALAAEGRGSSCEASVSFEDVTVDFSKEEWQHLDPAQRRL |
| | | YWDVTLENYSHLLSVGYQIPKSEAAFKLEQGEGPWMLEGEAPHQSC |
| | | SGEAIGKMQQQGIPGGIFFHC |
| 43 | ZNF189 | MASPSPPPESKEEWDYLDPAQRSLYKDVMMENYGNLVSLDVLNRDK |
| | | DEEPTVKQEIEEIEEEVEPQGVIVTRIKSEIDQDPMGRETFELVGR |
| | | LDKQRGIFLWEIPRESL |
| 44 | ZNF528 | MALTQGPLKFMDVAIEFSQEEWKCLDPAQRTLYRDVMLENYRNLVS |
| | | LGICLPDLSVTSMLEQKRDPWTLQSEEKIANDPDGRECIKGVNTER |
| | | SSKLGSN |
| 45 | ZNF543 | MAASAQVSVTFEDVAVTFTQEEWGQLDAAQRTLYQEVMLETCGLLM |
| | | SLGCPLFKPELIYQLDHRQELWMATKDLSQSSYPGDNTKPKTTEPT |
| | | FSHLALPE |
| 46 | ZNF554 | MFSQEERMAAGYLPRWSQELVTFEDVSMDFSQEEWELLEPAQKNLY |
| | | REVMLENYRNVVSLEALKNQCTDVGIKEGPLSPAQTSQVTSLSSWT |
| | | GYLLFQPVASSHLEQREALWIEEKGTPQASCSDWMTVLRNQDSTYK |
| | | KVALQE |
| 47 | ZNF140 | MSQGSVTFRDVAIDFSQEEWKWLQPAQRDLYRCVMLENYGHLVSLG |
| | | LSISKPDVVSLLEQGKEPWLGKREVKRDLFSVSESSGEIKDFSPKN |
| | | VIYDD |
| 48 | ZNF610 | MEEAQKRKAKESGMALPQGRLTFMDVAIEFSQEEWKSLDPGQRALY |
| | | RDVMLENYRNLVFLGRSCVLGSNAENKPIKNQLGLTLESHLSELQL |
| | | FQAGRKIYRSNQVEKFTNHR |
| 49 | ZNF264 | MAAAVLTDRAQVSVTFDDVAVTFTKEEWGQLDLAQRTLYQEVMLEN |
| | | CGLLVSLGCPVPKAELICHLEHGQEPWTRKEDLSQDTCPGDKGKPK |
| | | TTEPTTCEPALSE |
| 50 | ZNF350 | MIQAQESITLEDVAVDFTWEEWQLLGAAQKDLYRDVMLENYSNLVA |
| | | VGYQASKPDALFKLEQGEQLWTIEDGIHSGACSDIWKVDHVLERLQ |
| | | SESLVNR |
| 51 | ZNF8 | MEGVAGVMSVGPPAARLQEPVTFRDVAVDFTQEEWGQLDPTQRILY |
| | | RDVMLETFGHLLSIGPELPKPEVISQLEQGTELWVAERGTTQGCHP |
| | | AWEPRSESQASRKEEGLPEE |
| 52 | ZNF582 | MSLGSELFRDVAIVFSQEEWQWLAPAQRDLYRDVMLETYSNLVSLG |
| | | LAVSKPDVISFLEQGKEPWMVERVVSGGLCPVLESRYDTKELFPKQ |
| | | HVYEV |
| 53 | ZNF30 | MAHKYVGLQYHGSVTFEDVAIAFSQQEWESLDSSQRGLYRDVMLEN |
| | | YRNLVSMAGHSRSKPHVIALLEQWKEPEVTVRKDGRRWCTDLQLED |
| | | DTIGCKEMPTSEN |
| 54 | ZNF324 | MAFEDVAVYFSQEEWGLLDTAQRALYRRVMLDNFALVASLGLSTSR |
| | | PRVVIQLERGEEPWVPSGTDTTLSRTTYRRRNPGSWSLTEDRDVSG |
| 55 | ZNF98 | MLENYRNLVFVGIAASKPDLITCLEQGKEPWNVKRHEMVTEPPVVY |
| | | SYFAQDLWPKQGKKNYFQKVILRTYKKCGRENLQLRKYCKSMDECK |
| | | VHKECYNGLNQC |
| 56 | ZNF669 | MHFRRPDPCREPLASPIQDSVAFEDVAVNFTQEEWALLDSSQKNLY |
| | | REVMQETCRNLASVGSQWKDQNIEDHFEKPGKDIRNHIVQRLCESK |
| | | EDGQYGEVVSQIPNLDLNENISTGLKPCECSICGK |
| 57 | ZNF677 | MALSQGLFTFKDVAIEFSQEEWECLDPAQRALYRDVMLENYRNLLS |
| | | LDEDNIPPEDDISVGFTSKGLSPKENNKEELYHLVILERKESHGIN |
| | | NFDLKEVWENMPKFDSLW |
| 58 | ZNF596 | MTFEDIIVDFTQEEWALLDTSQRKLFQDVMLENISHLVSIGKQLCK |
| | | SVVLSQLEQVEKLSTQRISLLQGREVGIKHQEIPFIHHIYQKGTST |
| | | ISTMRS |
| 59 | ZNF214 | MAVTFEDVTIIFTWEEWKFLDSSQKRLYREVMWENYTNVMSVENWN |
| | | ESYKSQEEKFRYLEYENFSYWQGWWNAGAQMYENQNYGETVQGTDS |
| | | KDLTQQDRSQC |
| 60 | ZNF37A | MITSQGSVSFRDVTVGFTQEEWQHLDPAQRTLYRDVMLENYSHLVS |
| | | VGYCIPKPEVILKLEKGEEPWILEEKFPSQSHLELINTSRNYSIMK |
| | | FNEFNKG |
| 61 | ZNF34 | MFEDVAVYLSREEWGRLGPAQRGLYRDVMLETYGNLVSLGVGPAGP |
| | | KPGVISQLERGDEPWVLDVQGTSGKEHLRVNSPALGTRTEYKELTS |
| | | QETFGEEDPQGSEPVEACDHIS |
| 62 | ZNF250 | METYGNVVSLGLPGSKPDIISQLERGEDPWVLDRKGAKKSQGLWSD |
| | | YSDNLKYDHTTACTQQDSLSCPWECETKGESQNTDLSPKPLISEQT |
| | | VILGKTPLGRIDQENNETKQ |
| 63 | ZNF547 | MAEMNPAQGHVVFEDVAIYFSQEEWGHLDEAQRLLYRDVMLENLAL |
| | | LSSLGCCHGAEDEEAPLEPGVSVGVSQVMAPKPCLSTQNTQPCETC |
| | | SSLLKDILRL |
| 64 | ZNF273 | MLDNYRNLVFLGIAVSKPDLITCLEQGKEPCNMKRHAMVAKPPVVC |
| | | SHFAQDLWPKQGLKDS |
| 65 | ZNF354A | MAAGQREARPQVSLTFEDVAVLFTRDEWRKLAPSQRNLYRDVMLEN |
| | | YRNLVSLGLPFTKPKVISLLQQGEDPWEVEKDGSGVSSLGSKSSHK |
| | | TTKSTQTQDSSFQ |
| 66 | ZFP82 | MALRSVMFSDVSIDFSPEEWEYLDLEQKDLYRDVMLENYSNLVSLG |
| | | CFISKPDVISSLEQGKEPWKVVRKGRRQYPDLETKYETKKLSLEND |
| | | IYEIN |
| 67 | ZNF224 | MTTFKEAMTFKDVAVVFTEEELGLLDLAQRKLYRDVMLENFRNLLS |
| | | VGHQAFHRDTFHFLREEKIWMMKTAIQREGNSGDKIQTEMETVSEA |
| | | GTHQEW |
| 68 | ZNF33A | MFQVEQKSQESVSFKDVTVGFTQEEWQHLDPSQRALYRDVMLENYS |
| | | NLVSVGYCVHKPEVIFRLQQGEEPWKQEEEFPSQSFPEVWTADHLK |
| | | ERSQENQSKHL |
| 69 | ZNF45 | MTKSKEAVTFKDVAVVFSEEELQLLDLAQRKLYRDVMLENFRNVVS |
| | | VGHQSTPDGLPQLEREEKLWMMKMATQRDNSSGAKNLKEMETLQEV |
| | | GLRYLP |
| 70 | ZNF175 | MSQKPQVLGPEKQDGSCEASVSFEDVTVDFSREEWQQLDPAQRCLY |
| | | RDVMLELYSHLFAVGYHIPNPEVIFRMLKEKEPRVEEAEVSHQRCQ |
| | | EREFGLEIPQKEISKKASFQ |
| 71 | ZNF595 | MELVTFRDVAIEFSPEEWKCLDPAQQNLYRDVMLENYRNLVSLGFV |
| | | ISNPDLVTCLEQIKEPCNLKIHETAAKPPAICSPFSQDLSPVQGIE |
| | | DSF |
| 72 | ZNF184 | MSTLLQGGHNLLSSASFQESVTFKDVIVDFTQEEWKQLDPGQRDLF |
| | | RDVTLENYTHLVSIGLQVSKPDVISQLEQGTEPWIMEPSIPVGTCA |
| | | DWETRLENSVSAPEPDISEE |
| 73 | ZNF419 | MDPAQVPVAADLLTDHEEGYVTFEDVAVYFSQEEWRLLDDAQRLLY |
| | | RNVMLENFTLLASLGLASSKTHEITQLESWEEPFMPAWEVVTSAIP |
| | | RGCWHGAEAEEAPEQIASVG |
| 74 | ZFP28-1 | MKKLEAVGTGIEPKAMSQGLVTFGDVAVDFSQEEWEWLNPIQRNLY |
| | | RKVMLENYRNLASLGLCVSKPDVISSLEQGKEPWTVKRKMTRAWCP |
| | | DLKAVWKIKELPLKKDFCEG |
| 75 | ZFP28-2 | MSLLGEHWDYDALFETQPGLVTIKNLAVDFRQQLHPAQKNFCKNGI |
| | | WENNSDLGSAGHCVAKPDLVSLLEQEKEPWMVKRELTGSLFSGQRS |
| | | VHETQELFPKQDSYAE |
| 76 | ZNF18 | MLALAASQPARLEERLIRDRDLGASLLPAAPQEQWRQLDSTQKEQY |
| | | WDLILETYGKMVSGAGISHPKSDLINSIEFGEELAGIYLHVNEKIP |
| | | RPTCIGDRQENDKENLNLENH |
| 77 | ZNF213 | MEGRPGETTDTCFVSGVHGPVALGDIPFYFSREEWGTLDPAQRDLF |
| | | WDIKRENSRNTTLGFGLKGQSEKSLLQEMVPVVPGQTGSDVTVSWS |
| | | PEEAEAWESENRPRAALGPVVGARRGRPPTRRRQFRDLA |
| 78 | ZNF394 | MVAVVRALQRALDGTSSQGMVTFEDTAVSLTWEEWERLDPARRDFC |
| | | RESAQKDSGSTVPPSLESRVENKELIPMQQILEEAEPQGQLQEAFQ |
| | | GKRPLFSKCGSTHEDRVEKQSGDP |
| 79 | ZFP1 | MNKSQGSVSFTDVTVDFTQEEWEQLDPSQRILYMDVMLENYSNLLS |
| | | VEVWKADDQMERDHRNPDEQARQFLILKNQTPIEERGDLFGKALNL |
| | | NTDFVSLRQVPYKYDLYEKTL |
| 80 | ZFP14 | MAHGSVTFRDVAIDFSQEEWEFLDPAQRDLYRDVMWENYSNFISLG |
| | | PSISKPDVITLLDEERKEPGMVVREGTRRYCPDLESRYRINTLSPE |
| | | KDIYEIYSFQWDIMER |
| 81 | ZNF416 | MAAAVLRDSTSVPVTAEAKLMGFTQGCVTFEDVAIYFSQEEWGLLD |
| | | EAQRLLYRDVMLENFALITALVCWHGMEDEETPEQSVSVEGVPQVR |
| | | TPEASPSTQKIQSCDMCVPFLTDILHLTDLPGQELYLTGACAVEHQ |
| | | DQK |
| 82 | ZNF557 | MLPPTAASQREGHTEGGELVNELLKSWLKGLVTFEDVAVEFTQEEW |
| | | ALLDPAQRTLYRDVMLENCRNLASLGNQVDKPRLISQLEQEDKVMT |
| | | EERGILSGTCPDVENPFKAKGLTPKLHVFRKEQSRNMKMER |
| 83 | ZNF566 | MAQESVMFSDVSVDFSQEEWECLNDDQRDLYRDVMLENYSNLVSMG |
| | | HSISKPNVISYLEQGKEPWLADRELTRGQWPVLESRCETKKLFLKK |
| | | EIYEIESTQWEIMEK |
| 84 | ZNF729 | MPGAPGSLEMGPLTFRDVTIEFSLEEWQCLDTVQQNLYRDVMLENY |
| | | RNLVFLGMAVFKPDLITCLKQGKEPWNMKRHEMVTKPPVMRSHFTQ |
| | | DLWPDQSTKDSFQEVILRTYAR |
| 85 | ZIM2 | MAGSQFPDFKHLGTFLVFEELVTFEDVLVDFSPEELSSLSAAQRNL |
| | | YREVMLENYRNLVSLGHQFSKPDIISRLEEEESYAMETDSRHTVIC |
| | | QGE |
| 86 | ZNF254 | MPGPPRSLEMGLLTFRDVAIEFSLEEWQHLDIAQQNLYRNVMLENY |
| | | RNLAFLGIAVSKPDLITCLEQGKEPWNMKRHE |
| 87 | ZNF764 | MAPPLAPLPPRDPNGAGPEWREPGAVSFADVAVYFCREEWGCLRPA |
| | | QRALYRDVMRETYGHLSALGIGGNKPALISWVEEEAELWGPAAQDP |
| | | E |
| 88 | ZNF785 | MGPPLAPRPAHVPGEAGPRRTRESRPGAVSFADVAVYFSPEEWECL |
| | | RPAQRALYRDVMRETFGHLGALGFSVPKPAFISWVEGEVEAWSPEA |
| | | QDPDGESS |
| 89 | ZNF10 (KOX1) | MDAKSLTAWSRTLVTFKDVFVDFTREEWKLLDTAQQIVYRNVMLEN |
| | | YKNLVSLGYQLTKPDVILRLEKGEEPWLVEREIHQETHPDSETAFE |
| | | IKSSVSSRSIFKDKQSCDIKMEGMARNDLWYLSLEEVWKCRDQLDK |
| | | YQENPERHLRQVAFTQKKVLTQERVSESGKYGGNCLLPAQLVLREY |
| | | FHKRDSHTKSLKHDLVLNGHQDSCASNSNECGQTFCQNIHLIQFAR |
| | | THTGDKSYKCPDNDNSLTHGSSLGISKGIHREKPYECKECGKFFSW |
| | | RSNLTRHQLIHTGEKPYECKECGKSFSRSSHLIGHQKTHTGEEPYE |
| | | CKECGKSFSWFSHLVTHQRTHTGDKLYTCNQCGKSFVHSSRLIRHQ |
| | | RTHTGEKPYECPECGKSFRQSTHLILHQRTHVRVRPYECNECGKSY |
| | | SQRSHLVVHHRIHTGLKPFECKDCGKCFSRSSHLYSHQRTHTGEKP |
| | | YECHDCGKSFSQSSALIVHQRIHTGEKPYECCQCGKAFIRKNDLIK |
| | | HQRIHVGEETYKCNQCGIIFSQNSPFIVHQIAHTGEQFLTCNQCGT |
| | | ALVNTSNLIGYQTNHIRENAY |
| 90 | CBX5 | MGKKTKRTADSSSSEDEEEYVVEKVLDRRVVKGQVEYLLKWKGFSE |
| | (chromoshadow | EHNTWEPEKNLDCPELISEFMKKYKKMKEGENNKPREKSESNKRKS |
| | domain) | NFSNSADDIKSKKKREQSNDIARGFERGLEPEKIIGATDSCGDLMF |
| | | LMKWKDTDEADLVLAKEANVKCPQIVIAFYEERLTWHAYPEDAENK |
| | | EKETAKS |
| 91 | RYBP | MTMGDKKSPTRPKRQAKPAADEGFWDCSVCTERNSAEAFKCSICDV |
| | (YAF2_RYBP | RKGTSTRKPRINSQLVAQQVAQQYATPPPPKKEKKEKVEKQDKEKP |
| | component of | EKDKEISPSVTKKNTNKKTKPKSDILKDPPSEANSIQSANATTKTS |
| | PRC1) | ETNHTSRPRLKNVDRSTAQQLAVTVGNVTVIITDFKEKTRSSSTSS |
| | | STVTSSAGSEQQNQSSSGSESTDKGSSRSSTPKGDMSAVNDESF |
| 92 | YAF2 | MGDKKSPTRPKRQPKPSSDEGYWDCSVCTFRNSAEAFKCMMCDVRK |
| | (YAF2_RYBP | GTSTRKPRPVSQLVAQQVTQQFVPPTQSKKEKKDKVEKEKSEKETT |
| | component of | SKKNSHKKTRPRLKNVDRSSAQHLEVTVGDLTVIITDFKEKTKSPP |
| | PRC1) | ASSAASADQHSQSGSSSDNTERGMSRSSSPRGEASSLNGESH |
| 93 | MGA (component | MEEKQQIILANQDGGTVAGAAPTFFVILKQPGNGKTDQGILVTNQD |
| | of PRC1.6) | ACALASSVSSPVKSKGKICLPADCTVGGITVTLDNNSMWNEFYHRS |
| | | TEMILTKQGRRMFPYCRYWITGLDSNLKYILVMDISPVDNHRYKWN |
| | | GRWWEPSGKAEPHVLGRVFIHPESPSTGHYWMHQPVSFYKLKLINN |
| | | TLDQEGHIILHSMHRYLPRLHLVPAEKAVEVIQLNGPGVHTFTFPQ |
| | | TEFFAVTAYQNIQITQLKIDYNPFAKGFRDDGLNNKPQRDGKQKNS |
| | | SDQEGNNISSSSGHRVRLTEGQGSEIQPGDLDPLSRGHETSGKGLE |
| | | KTSLNIKRDFLGFMDTDSALSEVPQLKQEISECLIASSFEDDSRVA |
| | | SPLDQNGSFNVVIKEEPLDDYDYELGECPEGVTVKQEETDEETDVY |
| | | SNSDDDPILEKQLKRHNKVDNPEADHLSSKWLPSSPSGVAKAKMFK |
| | | LDTGKMPVVYLEPCAVTRSTVKISELPDNMLSTSRKDKSSMLAELE |
| | | YLPTYIENSNETAFCLGKESENGLRKHSPDLRVVQKYPLLKEPQWK |
| | | YPDISDSISTERILDDSKDSVGDSLSGKEDLGRKRTTMLKIATAAK |
| | | VVNANQNASPNVPGKRGRPRKLKLCKAGRPPKNTGKSLISTKNTPV |
| | | SPGSTFPDVKPDLEDVDGVLFVSFESKEALDIHAVDGTTEESSSLQ |
| | | ASTTNDSGYRARISQLEKELIEDLKTLRHKQVIHPGLQEVGLKLNS |
| | | VDPTMSIDLKYLGVQLPLAPATSFPFWNLTGTNPASPDAGFPFVSR |
| | | TGKINDFTKIKGWRGKFHSASASRNEGGNSESSLKNRSAFCSDKLD |
| | | EYLENEGKLMETSMGFSSNAPTSPVVYQLPTKSTSYVRTLDSVLKK |
| | | QSTISPSTSYSLKPHSVPPVSRKAKSQNRQATFSGRTKSSYKSILP |
| | | YPVSPKQKYSHVILGDKVTKNSSGIISENQANNFVVPTLDENIFPK |
| | | QISLRQAQQQQQQQQGSRPPGLSKSQVKLMDLEDCALWEGKPRTYI |
| | | TEERADVSLTILLTAQASLKTKPIHTIIRKRAPPCNNDFCRLGCVC |
| | | SSLALEKRQPAHCRRPDCMFGCTCLKRKVVLVKGGSKTKHFQRKAA |
| | | HRDPVFYDTLGEEAREEEEGIREEEEQLKEKKKRKKLEYTICETEP |
| | | EQPVRHYPLWVKVEGEVDPEPVYIPTPSVIEPMKPLLLPQPEVLSP |
| | | TVKGKLLTGIKSPRSYTPKPNPVIREEDKDPVYLYFESMMTCARVR |
| | | VYERKKEDQRQPSSSSSPSPSFQQQTSCHSSPENHNNAKEPDSEQQ |
| | | PLKQLTCDLEDDSDKLQEKSWKSSCNEGESSSTSYMHQRSPGGPTK |
| | | LIEIISDCNWEEDRNKILSILSQHINSNMPQSLKVGSFIIELASQR |
| | | KSRGEKNPPVYSSRVKISMPSCQDQDDMAEKSGSETPDGPLSPGKM |
| | | EDISPVQTDALDSVRERLHGGKGLPFYAGLSPAGKLVAYKRKPSSS |
| | | TSGLIQVASNAKVAASRKPRTLLPSTSNSKMASSSGTATNRPGKNL |
| | | KAFVPAKRPIAARPSPGGVFTQFVMSKVGALQQKIPGVSTPQTLAG |
| | | TQKFSIRPSPVMVVTPVVSSEPVQVCSPVTAAVTTTTPQVFLENTT |
| | | AVTPMTAISDVETKETTYSSGATTTGVVEVSETNTSTSVTSTQSTA |
| | | TVNLTKTTGITTPVASVAFPKSLVASPSTITLPVASTASTSLVVVT |
| | | AAASSSMVTTPTSSLGSVPIILSGINGSPPVSQRPENAAQIPVATP |
| | | QVSPNTVKRAGPRLLLIPVQQGSPTLRPVSNTQLQGHRMVLQPVRS |
| | | PSGMNLFRHPNGQIVQLLPLHQLRGSNTQPNLQPVMFRNPGSVMGI |
| | | RLPAPSKPSETPPSSTSSSAFSVMNPVIQAVGSSSAVNVITQAPSL |
| | | LSSGASFVSQAGTLTLRISPPEPQSFASKTGSETKITYSSGGQPVG |
| | | TASLIPLQSGSFALLQLPGQKPVPSSILQHVASLQMKRESQNPDQK |
| | | DETNSIKREQETKKVLQSEGEAVDPEANVIKQNSGAATSEETLNDS |
| | | LEDRGDHLDEECLPEEGCATVKPSEHSCITGSHTDQDYKDVNEEYG |
| | | ARNRKSSKEKVAVLEVRTISEKASNKTVQNLSKVQHQKLGDVKVEQ |
| | | QKGFDNPEENSSEFPVTFKEESKFELSGSKVMEQQSNLQPEAKEKE |
| | | CGDSLEKDRERWRKHLKGPLTRKCVGASQECKKEADEQLIKETKTC |
| | | QENSDVFQQEQGISDLLGKSGITEDARVLKTECDSWSRISNPSAFS |
| | | IVPRRAAKSSRGNGHFQGHLLLPGEQIQPKQEKKGGRSSADFTVLD |
| | | LEEDDEDDNEKTDDSIDEIVDVVSDYQSEEVDDVEKNNCVEYIEDD |
| | | EEHVDIETVEELSEEINVAHLKTTAAHTQSFKQPSCTHISADEKAA |
| | | ERSRKAPPIPLKLKPDYWSDKLQKEAEAFAYYRRTHTANERRRRGE |
| | | MRDLFEKLKITLGLLHSSKVSKSLILTRAFSEIQGLTDQADKLIGQ |
| | | KNLLTRKRNILIRKVSSLSGKTEEVVLKKLEYIYAKQQALEAQKRK |
| | | KKMGSDEFDISPRISKQQEGSSASSVDLGQMFINNRRGKPLILSRK |
| | | KDQATENTSPLNTPHTSANLVMTPQGQLLTLKGPLFSGPVVAVSPD |
| | | LLESDLKPQVAGSAVALPENDDLFMMPRIVNVTSLATEGGLVDMGG |
| | | SKYPHEVPDSKPSDHLKDTVRNEDNSLEDKGRISSRGNRDGRVTLG |
| | | PTQVFLANKDSGYPQIVDVSNMQKAQEFLPKKISGDMRGIQYKWKE |
| | | SESRGERVKSKDSSFHKLKMKDLKDSSIEMELRKVTSAIEEAALDS |
| | | SELLTNMEDEDDTDETLTSLLNEIAFLNQQLNDDSVGLAELPSSMD |
| | | TEFPGDARRAFISKVPPGSRATFQVEHLGTGLKELPDVQGESDSIS |
| | | PLLLHLEDDDFSENEKQLAEPASEPDVLKIVIDSEIKDSLLSNKKA |
| | | IDGGKNTSGLPAEPESVSSPPTLHMKTGLENSNSTDTLWRPMPKLA |
| | | PLGLKVANPSSDADGQSLKVMPCLAPIAAKVGSVGHKMNLTGNDQE |
| | | GRESKVMPTLAPVVAKLGNSGASPSSAGK |
| 94 | CBX1 | MGKKQNKKKVEEVLEEEEEEYVVEKVLDRRVVKGKVEYLLKWKGFS |
| | (chromoshadow) | DEDNTWEPEENLDCPDLIAEFLQSQKTAHETDKSEGGKRKADSDSE |
| | | DKGEESKPKKKKEESEKPRGFARGLEPERIIGATDSSGELMFLMKW |
| | | KNSDEADLVPAKEANVKCPQVVISFYEERLTWHSYPSEDDDKKDDK |
| | | N |
| 95 | SCMH1 | MLVCYSVLACEILWDLPCSIMGSPLGHFTWDKYLKETCSVPAPVHC |
| | (SAM_1/SPM) | FKQSYTPPSNEFKISMKLEAQDPRNTTSTCIATVVGLTGARLRLRL |
| | | DGSDNKNDFWRLVDSAEIQPIGNCEKNGGMLQPPLGFRLNASSWPM |
| | | FLLKTLNGAEMAPIRIFHKEPPSPSHNFFKMGMKLEAVDRKNPHFI |
| | | CPATIGEVRGSEVLVTFDGWRGAFDYWCRFDSRDIFPVGWCSLTGD |
| | | NLQPPGTKVVIPKNPYPASDVNTEKPSIHSSTKTVLEHQPGQRGRK |
| | | PGKKRGRTPKTLISHPISAPSKTAEPLKFPKKRGPKPGSKRKPRTL |
| | | LNPPPASPTTSTPEPDTSTVPQDAATIPSSAMQAPTVCIYLNKNGS |
| | | TGPHLDKKKVQQLPDHFGPARASVVLQQAVQACIDCAYHQKTVFSF |
| | | LKQGHGGEVISAVFDREQHTLNLPAVNSITYVLRFLEKLCHNLRSD |
| | | NLFGNQPFTQTHLSLTAIEYSHSHDRYLPGETFVLGNSLARSLEPH |
| | | SDSMDSASNPTNLVSTSQRHRPLLSSCGLPPSTASAVRRLCSRGVL |
| | | KGSNERRDMESFWKLNRSPGSDRYLESRDASRLSGRDPSSWTVEDV |
| | | MQFVREADPQLGPHADLFRKHEIDGKALLLLRSDMMMKYMGLKLGP |
| | | ALKLSYHIDRLKQGKF |
| 96 | MPP8 | MEQVAEGARVTAVPVSAADSTEELAEVEEGVGVVGEDNDAAARGAE |
| | (Chromodomain) | AFGDSEEDGEDVFEVEKILDMKTEGGKVLYKVRWKGYTSDDDTWEP |
| | | EIHLEDCKEVLLEFRKKIAENKAKAVRKDIQRLSLNNDIFEANSDS |
| | | DQQSETKEDTSPKKKKKKLRQREEKSPDDLKKKKAKAGKLKDKSKP |
| | | DLESSLESLVFDLRTKKRISEAKEELKESKKPKKDEVKETKELKKV |
| | | KKGEIRDLKTKTREDPKENRKTKKEKFVESQVESESSVLNDSPFPE |
| | | DDSEGLHSDSREEKQNTKSARERAGQDMGLEHGFEKPLDSAMSAEE |
| | | DTDVRGRRKKKTPRKAEDTRENRKLENKNAFLEKKTVPKKQRNQDR |
| | | SKSAAELEKLMPVSAQTPKGRRLSGEERGLWSTDSAEEDKETKRNE |
| | | SKEKYQKRHDSDKEEKGRKEPKGLKTLKEIRNAFDLFKLTPEEKND |
| | | VSENNRKREEIPLDFKTIDDHKTKENKQSLKERRNTRDETDTWAYI |
| | | AAEGDQEVLDSVCQADENSDGRQQILSLGMDLQLEWMKLEDFQKHL |
| | | DGKDENFAATDAIPSNVLRDAVKNGDYITVKVALNSNEEYNLDQED |
| | | SSGMTLVMLAAAGGQDDLLRLLITKGAKVNGRQKNGTTALIHAAEK |
| | | NFLTTVAILLEAGAFVNVQQSNGETALMKACKRGNSDIVRLVIECG |
| | | ADCNILSKHQNSALHFAKQSNNVLVYDLLKNHLETLSRVAEETIKD |
| | | YFEARLALLEPVFPIACHRLCEGPDFSTDFNYKPPQNIPEGSGILL |
| | | FIFHANFLGKEVIARLCGPCSVQAVVLNDKFQLPVELDSHFVYSFS |
| | | PVAGPNKLFIRLTEAPSAKVKLLIGAYRVQLQ |
| 97 | SUMO3 (Rad60- | MSEEKPKEGVKTENDHINLKVAGQDGSVVQFKIKRHTPLSKLMKAY |
| | SLD) | CERQGLSMRQIRFRFDGQPINETDTPAQLEMEDEDTIDVFQQQTGG |
| | | VPESSLAGHSF |
| 98 | HERC2 (Cyt-b5) | MPSESFCLAAQARLDSKWLKTDIQLAFTRDGLCGLWNEMVKDGEIV |
| | | YTGTESTQNGELPPRKDDSVEPSGTKKEDLNDKEKKDEEETPAPIY |
| | | RAKSILDSWVWGKQPDVNELKECLSVLVKEQQALAVQSATTTLSAL |
| | | RLKQRLVILERYFIALNRTVFQENVKVKWKSSGISLPPVDKKSSRP |
| | | AGKGVEGLARVGSRAALSFAFAFLRRAWRSGEDADLCSELLQESLD |
| | | ALRALPEASLFDESTVSSVWLEVVERATRFLRSVVTGDVHGTPATK |
| | | GPGSIPLQDQHLALAILLELAVQRGTLSQMLSAILLLLQLWDSGAQ |
| | | ETDNERSAQGTSAPLLPLLQRFQSIICRKDAPHSEGDMHLLSGPLS |
| | | PNESFLRYLTLPQDNELAIDLRQTAVVVMAHLDRLATPCMPPLCSS |
| | | PTSHKGSLQEVIGWGLIGWKYYANVIGPIQCEGLANLGVTQIACAE |
| | | KRFLILSRNGRVYTQAYNSDTLAPQLVQGLASRNIVKIAAHSDGHH |
| | | YLALAATGEVYSWGCGDGGRLGHGDTVPLEEPKVISAFSGKQAGKH |
| | | VVHIACGSTYSAAITAEGELYTWGRGNYGRLGHGSSEDEAIPMLVA |
| | | GLKGLKVIDVACGSGDAQTLAVTENGQVWSWGDGDYGKLGRGGSDG |
| | | CKTPKLIEKLQDLDVVKVRCGSQFSIALTKDGQVYSWGKGDNQRLG |
| | | HGTEEHVRYPKLLEGLQGKKVIDVAAGSTHCLALTEDSEVHSWGSN |
| | | DQCQHFDTLRVTKPEPAALPGLDTKHIVGIACGPAQSFAWSSCSEW |
| | | SIGLRVPFVVDICSMTFEQLDLLLRQVSEGMDGSADWPPPQEKECV |
| | | AVATLNLLRLQLHAAISHQVDPEFLGLGLGSILLNSLKQTVVTLAS |
| | | SAGVLSTVQSAAQAVLQSGWSVLLPTAEERARALSALLPCAVSGNE |
| | | VNISPGRRFMIDLLVGSLMADGGLESALHAAITAEIQDIEAKKEAQ |
| | | KEKEIDEQEANASTFHRSRTPLDKDLINTGICESSGKQCLPLVQLI |
| | | QQLLRNIASQTVARLKDVARRISSCLDFEQHSRERSASLDLLLRFQ |
| | | RLLISKLYPGESIGQTSDISSPELMGVGSLLKKYTALLCTHIGDIL |
| | | PVAASIASTSWRHFAEVAYIVEGDFTGVLLPELVVSIVLLLSKNAG |
| | | LMQEAGAVPLLGGLLEHLDRFNHLAPGKERDDHEELAWPGIMESFF |
| | | TGQNCRNNEEVTLIRKADLENHNKDGGFWTVIDGKVYDIKDFQTQS |
| | | LTGNSILAQFAGEDPVVALEAALQFEDTRESMHAFCVGQYLEPDQE |
| | | IVTIPDLGSLSSPLIDTERNLGLLLGLHASYLAMSTPLSPVEIECA |
| | | KWLQSSIFSGGLQTSQIHYSYNEEKDEDHCSSPGGTPASKSRLCSH |
| | | RRALGDHSQAFLQAIADNNIQDHNVKDFLCQIERYCRQCHLTTPIM |
| | | FPPEHPVEEVGRLLLCCLLKHEDLGHVALSLVHAGALGIEQVKHRT |
| | | LPKSVVDVCRVVYQAKCSLIKTHQEQGRSYKEVCAPVIERLRFLEN |
| | | ELRPAVCNDLSIMSKFKLLSSLPRWRRIAQKIIRERRKKRVPKKPE |
| | | STDDEEKIGNEESDLEEACILPHSPINVDKRPIAIKSPKDKWQPLL |
| | | STVTGVHKYKWLKQNVQGLYPQSPLLSTIAEFALKEEPVDVEKMRK |
| | | CLLKQLERAEVRLEGIDTILKLASKNFLLPSVQYAMFCGWQRLIPE |
| | | GIDIGEPLTDCLKDVDLIPPENRMLLEVTFGKLYAWAVQNIRNVLM |
| | | DASAKFKELGIQPVPLQTITNENPSGPSLGTIPQARFLLVMLSMLT |
| | | LQHGANNLDLLLNSGMLALTQTALRLIGPSCDNVEEDMNASAQGAS |
| | | ATVLEETRKETAPVQLPVSGPELAAMMKIGTRVMRGVDWKWGDQDG |
| | | PPPGLGRVIGELGEDGWIRVQWDTGSTNSYRMGKEGKYDLKLAELP |
| | | AAAQPSAEDSDTEDDSEAEQTERNIHPTAMMFTSTINLLQTLCLSA |
| | | GVHAEIMQSEATKTLCGLLRMLVESGTTDKTSSPNRLVYREQHRSW |
| | | CTLGFVRSIALTPQVCGALSSPQWITLLMKVVEGHAPFTATSLQRQ |
| | | ILAVHLLQAVLPSWDKTERARDMKCLVEKLFDFLGSLLTTCSSDVP |
| | | LLRESTLRRRRVRPQASLTATHSSTLAEEVVALLRTLHSLTQWNGL |
| | | INKYINSQLRSITHSFVGRPSEGAQLEDYFPDSENPEVGGLMAVLA |
| | | VIGGIDGRLRLGGQVMHDEFGEGTVTRITPKGKITVQFSDMRTCRV |
| | | CPLNQLKPLPAVAFNVNNLPFTEPMLSVWAQLVNLAGSKLEKHKIK |
| | | KSTKQAFAGQVDLDLLRCQQLKLYILKAGRALLSHQDKLRQILSQP |
| | | AVQETGTVHTDDGAVVSPDLGDMSPEGPQPPMILLQQLLASATQPS |
| | | PVKAIFDKQELEAAALAVCQCLAVESTHPSSPGFEDCSSSEATTPV |
| | | AVQHIRPARVKRRKQSPVPALPIVVQLMEMGFSRRNIEFALKSLTG |
| | | ASGNASSLPGVEALVGWLLDHSDIQVTELSDADTVSDEYSDEEVVE |
| | | DVDDAAYSMSTGAVVTESQTYKKRADFLSNDDYAVYVRENIQVGMM |
| | | VRCCRAYEEVCEGDVGKVIKLDRDGLHDLNVQCDWQQKGGTYWVRY |
| | | IHVELIGYPPPSSSSHIKIGDKVRVKASVTTPKYKWGSVTHQSVGV |
| | | VKAFSANGKDIIVDFPQQSHWTGLLSEMELVPSIHPGVTCDGCQMF |
| | | PINGSRFKCRNCDDFDFCETCFKTKKHNTRHTFGRINEPGQSAVFC |
| | | GRSGKQLKRCHSSQPGMLLDSWSRMVKSLNVSSSVNQASRLIDGSE |
| | | PCWQSSGSQGKHWIRLEIFPDVLVHRLKMIVDPADSSYMPSLVVVS |
| | | GGNSLNNLIELKTININPSDTTVPLLNDCTEYHRYIEIAIKQCRSS |
| | | GIDCKIHGLILLGRIRAEEEDLAAVPFLASDNEEEEDEKGNSGSLI |
| | | RKKAAGLESAATIRTKVFVWGLNDKDQLGGLKGSKIKVPSFSETLS |
| | | ALNVVQVAGGSKSLFAVTVEGKVYACGEATNGRLGLGISSGTVPIP |
| | | RQITALSSYVVKKVAVHSGGRHATALTVDGKVFSWGEGDDGKLGHF |
| | | SRMNCDKPRLIEALKTKRIRDIACGSSHSAALTSSGELYTWGLGEY |
| | | GRLGHGDNTTQLKPKMVKVLLGHRVIQVACGSRDAQTLALTDEGLV |
| | | FSWGDGDFGKLGRGGSEGCNIPQNIERLNGQGVCQIECGAQFSLAL |
| | | TKSGVVWTWGKGDYFRLGHGSDVHVRKPQVVEGLRGKKIVHVAVGA |
| | | LHCLAVTDSGQVYAWGDNDHGQQGNGTTTVNRKPTLVQGLEGQKIT |
| | | RVACGSSHSVAWTTVDVATPSVHEPVLFQTARDPLGASYLGVPSDA |
| | | DSSAASNKISGASNSKPNRPSLAKILLSLDGNLAKQQALSHILTAL |
| | | QIMYARDAVVGALMPAAMIAPVECPSFSSAAPSDASAMASPMNGEE |
| | | CMLAVDIEDRLSPNPWQEKREIVSSEDAVTPSAVTPSAPSASARPF |
| | | IPVTDDLGAASIIAETMTKTKEDVESQNKAAGPEPQALDEFTSLLI |
| | | ADDTRVVVDLLKLSVCSRAGDRGRDVLSAVLSGMGTAYPQVADMLL |
| | | ELCVTELEDVATDSQSGRLSSQPVVVESSHPYTDDTSTSGTVKIPG |
| | | AEGLRVEFDRQCSTERRHDPLTVMDGVNRIVSVRSGREWSDWSSEL |
| | | RIPGDELKWKFISDGSVNGWGWRFTVYPIMPAAGPKELLSDRCVLS |
| | | CPSMDLVTCLLDFRLNLASNRSIVPRLAASLAACAQLSALAASHRM |
| | | WALQRLRKLLTTEFGQSININRLLGENDGETRALSFTGSALAALVK |
| | | GLPEALQRQFEYEDPIVRGGKQLLHSPFFKVLVALACDLELDTLPC |
| | | CAETHKWAWFRRYCMASRVAVALDKRTPLPRLFLDEVAKKIRELMA |
| | | DSENMDVLHESHDIFKREQDEQLVQWMNRRPDDWTLSAGGSGTIYG |
| | | WGHNHRGQLGGIEGAKVKVPTPCEALATLRPVQLIGGEQTLFAVTA |
| | | DGKLYATGYGAGGRLGIGGTESVSTPILLESIQHVFIKKVAVNSGG |
| | | KHCLALSSEGEVYSWGEAEDGKLGHGNRSPCDRPRVIESLRGIEVV |
| | | DVAAGGAHSACVTAAGDLYTWGKGRYGRLGHSDSEDQLKPKLVEAL |
| | | QGHRVVDIACGSGDAQTLCLTDDDTVWSWGDGDYGKLGRGGSDGCK |
| | | VPMKIDSLTGLGVVKVECGSQFSVALTKSGAVYTWGKGDYHRLGHG |
| | | SDDHVRRPRQVQGLQGKKVIAIATGSLHCVCCTEDGEVYTWGDNDE |
| | | GQLGDGTTNAIQRPRLVAALQGKKVNRVACGSAHTLAWSTSKPASA |
| | | GKLPAQVPMEYNHLQEIPIIALRNRLLLLHHLSELFCPCIPMEDLE |
| | | GSLDETGLGPSVGFDTLRGILISQGKEAAFRKVVQATMVRDRQHGP |
| | | VVELNRIQVKRSRSKGGLAGPDGTKSVFGQMCAKMSSFGPDSLLLP |
| | | HRVWKVKFVGESVDDCGGGYSESIAEICEELQNGLTPLLIVTPNGR |
| | | DESGANRDCYLLSPAARAPVHSSMFRFLGVLLGIAIRTGSPLSLNL |
| | | AEPVWKQLAGMSLTIADLSEVDKDFIPGLMYIRDNEATSEEFEAMS |
| | | LPFTVPSASGQDIQLSSKHTHITLDNRAEYVRLAINYRLHEFDEQV |
| | | AAVREGMARVVPVPLLSLFTGYELETMVCGSPDIPLHLLKSVATYK |
| | | GIEPSASLIQWFWEVMESESNTERSLFLRFVWGRTRLPRTIADFRG |
| | | RDFVIQVLDKYNPPDHFLPESYTCFFLLKLPRYSCKQVLEEKLKYA |
| | | IHFCKSIDTDDYARIALTGEPAADDSSDDSDNEDVDSFASDSTQDY |
| | | LTGH |
| 99 | BIN1 (SH3_9) | MAEMGSKGVTAGKIASNVQKKLTRAQEKVLQKLGKADETKDEQFEQ |
| | | CVQNFNKQLTEGTRLQKDLRTYLASVKAMHEASKKLNECLQEVYEP |
| | | DWPGRDEANKIAENNDLLWMDYHQKLVDQALLTMDTYLGQFPDIKS |
| | | RIAKRGRKLVDYDSARHHYESLQTAKKKDEAKIAKPVSLLEKAAPQ |
| | | WCQGKLQAHLVAQTNLLRNQAEEELIKAQKVFEEMNVDLQEELPSL |
| | | WNSRVGFYVNTFQSIAGLEENFHKEMSKLNQNLNDVLVGLEKQHGS |
| | | NTFTVKAQPSDNAPAKGNKSPSPPDGSPAATPEIRVNHEPEPAGGA |
| | | TPGATLPKSPSQLRKGPPVPPPPKHTPSKEVKQEQILSLFEDTFVP |
| | | EISVTTPSQFEAPGPFSEQASLLDLDFDPLPPVTSPVKAPTPSGQS |
| | | IPWDLWEPTESPAGSLPSGEPSAAEGTFAVSWPSQTAEPGPAQPAE |
| | | ASEVAGGTQPAAGAQEPGETAASEAASSSLPAVVVETFPATVNGTV |
| | | EGGSGAGRLDLPPGFMFKVQAQHDYTATDTDELQLKAGDVVLVIPF |
| | | QNPEEQDEGWLMGVKESDWNQHKELEKCRGVFPENFTERVP |
| 100 | PCGF2 (RING | MHRTTRIKITELNPHLMCALCGGYFIDATTIVECLHSFCKTCIVRY |
| | finger protein | LETNKYCPMCDVQVHKTRPLLSIRSDKTLQDIVYKLVPGLFKDEMK |
| | domain) | RRRDFYAAYPLTEVPNGSNEDRGEVLEQEKGALSDDEIVSLSIEFY |
| | | EGARDRDEKKGPLENGDGDKEKTGVRFLRCPAAMTVMHLAKFLRNK |
| | | MDVPSKYKVEVLYEDEPLKEYYTLMDIAYIYPWRRNGPLPLKYRVQ |
| | | PACKRLTLATVPTPSEGTNTSGASECESVSDKAPSPATLPATSSSL |
| | | PSPATPSHGSPSSHGPPATHPTSPTPPSTASGATTAANGGSLNCLQ |
| | | TPSSTSRGRKMTVNGAPVPPLT |
| 101 | TOX (HMG box) | MDVRFYPPPAQPAAAPDAPCLGPSPCLDPYYCNKFDGENMYMSMTE |
| | | PSQDYVPASQSYPGPSLESEDFNIPPITPPSLPDHSLVHLNEVESG |
| | | YHSLCHPMNHNGLLPFHPQNMDLPEITVSNMLGQDGTLLSNSISVM |
| | | PDIRNPEGTQYSSHPQMAAMRPRGQPADIRQQPGMMPHGQLTTINQ |
| | | SQLSAQLGLNMGGSNVPHNSPSPPGSKSATPSPSSSVHEDEGDDTS |
| | | KINGGEKRPASDMGKKPKTPKKKKKKDPNEPQKPVSAYALFFRDTQ |
| | | AAIKGQNPNATFGEVSKIVASMWDGLGEEQKQVYKKKTEAAKKEYL |
| | | KQLAAYRASLVSKSYSEPVDVKTSQPPQLINSKPSVFHGPSQAHSA |
| | | LYLSSHYHQQPGMNPHLTAMHPSLPRNIAPKPNNQMPVTVSIANMA |
| | | VSPPPPLQISPPLHQHLNMQQHQPLTMQQPLGNQLPMQVQSALHSP |
| | | TMQQGFTLQPDYQTIINPTSTAAQVVTQAMEYVRSGCRNPPPQPVD |
| | | WNNDYCSSGGMQRDKALYLT |
| 102 | FOXA1 (HNF3A | MLGTVKMEGHETSDWNSYYADTQEAYSSVPVSNMNSGLGSMNSMNT |
| | C-terminal | YMTMNTMTTSGNMTPASFNMSYANPGLGAGLSPGAVAGMPGGSAGA |
| | domain) | MNSMTAAGVTAMGTALSPSGMGAMGAQQAASMNGLGPYAAAMNPCM |
| | | SPMAYAPSNLGRSRAGGGGDAKTFKRSYPHAKPPYSYISLITMAIQ |
| | | QAPSKMLTLSEIYQWIMDLFPYYRQNQQRWQNSIRHSLSENDCFVK |
| | | VARSPDKPGKGSYWTLHPDSGNMFENGCYLRRQKRFKCEKQPGAGG |
| | | GGGSGSGGSGAKGGPESRKDPSGASNPSADSPLHRGVHGKTGQLEG |
| | | APAPGPAASPQTLDHSGATATGGASELKTPASSTAPPISSGPGALA |
| | | SVPASHPAHGLAPHESQLHLKGDPHYSFNHPFSINNLMSSSEQQHK |
| | | LDFKAYEQALQYSPYGSTLPASLPLGSASVTTRSPIEPSALEPAYY |
| | | QGVYSRPVLNTS |
| 103 | FOXA2 (HNF3B | MLGAVKMEGHEPSDWSSYYAEPEGYSSVSNMNAGLGMNGMNTYMSM |
| | C-terminal | SAAAMGSGSGNMSAGSMNMSSYVGAGMSPSLAGMSPGAGAMAGMGG |
| | domain) | SAGAAGVAGMGPHLSPSLSPLGGQAAGAMGGLAPYANMNSMSPMYG |
| | | QAGLSRARDPKTYRRSYTHAKPPYSYISLITMAIQQSPNKMLTLSE |
| | | IYQWIMDLFPFYRQNQQRWQNSIRHSLSFNDCFLKVPRSPDKPGKG |
| | | SFWTLHPDSGNMFENGCYLRRQKRFKCEKQLALKEAAGAAGSGKKA |
| | | AAGAQASQAQLGEAAGPASETPAGTESPHSSASPCQEHKRGGLGEL |
| | | KGTPAAALSPPEPAPSPGQQQQAAAHLLGPPHHPGLPPEAHLKPEH |
| | | HYAFNHPFSINNLMSSEQQHHHSHHHHQPHKMDLKAYEQVMHYPGY |
| | | GSPMPGSLAMGPVTNKTGLDASPLAADTSYYQGVYSRPIMNSS |
| 104 | IRF2BP1 (IRF- | MASVQASRRQWCYLCDLPKMPWAMVWDFSEAVCRGCVNFEGADRIE |
| | 2BP1_2 N- | LLIDAARQLKRSHVLPEGRSPGPPALKHPATKDLAAAAAQGPQLPP |
| | terminal domain) | PQAQPQPSGTGGGVSGQDRYDRATSSGRLPLPSPALEYTLGSRLAN |
| | | GLGREEAVAEGARRALLGSMPGLMPPGLLAAAVSGLGSRGLTLAPG |
| | | LSPARPLFGSDFEKEKQQRNADCLAELNEAMRGRAEEWHGRPKAVR |
| | | EQLLALSACAPFNVRFKKDHGLVGRVFAFDATARPPGYEFELKLFT |
| | | EYPCGSGNVYAGVLAVARQMFHDALREPGKALASSGFKYLEYERRH |
| | | GSGEWRQLGELLTDGVRSFREPAPAEALPQQYPEPAPAALCGPPPR |
| | | APSRNLAPTPRRRKASPEPEGEAAGKMTTEEQQQRHWVAPGGPYSA |
| | | ETPGVPSPIAALKNVAEALGHSPKDPGGGGGPVRAGGASPAASSTA |
| | | QPPTQHRLVARNGEAEVSPTAGAEAVSGGGSGTGATPGAPLCCTLC |
| | | RERLEDTHFVQCPSVPGHKFCFPCSREFIKAQGPAGEVYCPSGDKC |
| | | PLVGSSVPWAFMQGEIATILAGDIKVKKERDP |
| 105 | IRF2BP2 (IRF- | MAAAVAVAAASRRQSCYLCDLPRMPWAMIWDFTEPVCRGCVNYEGA |
| | 2BP1_2 N- | DRVEFVIETARQLKRAHGCFPEGRSPPGAAASAAAKPPPLSAKDIL |
| | terminal domain) | LQQQQQLGHGGPEAAPRAPQALERYPLAAAAERPPRLGSDFGSSRP |
| | | AASLAQPPTPQPPPVNGILVPNGFSKLEEPPELNRQSPNPRRGHAV |
| | | PPTLVPLMNGSATPLPTALGLGGRAAASLAAVSGTAAASLGSAQPT |
| | | DLGAHKRPASVSSSAAVEHEQREAAAKEKQPPPPAHRGPADSLSTA |
| | | AGAAELSAEGAGKSRGSGEQDWVNRPKTVRDILLALHQHGHSGPFE |
| | | SKFKKEPALTAGRLLGFEANGANGSKAVARTARKRKPSPEPEGEVG |
| | | PPKINGEAQPWLSTSTEGLKIPMTPTSSFVSPPPPTASPHSNRTTP |
| | | PEAAQNGQSPMAALILVADNAGGSHASKDANQVHSTTRRNSNSPPS |
| | | PSSMNQRRLGPREVGGQGAGNTGGLEPVHPASLPDSSLATSAPLCC |
| | | TLCHERLEDTHEVQCPSVPSHKFCFPCSRQSIKQQGASGEVYCPSG |
| | | EKCPLVGSNVPWAFMQGEIATILAGDVKVKKERDS |
| 106 | IRF2BPLIRF- | MSAAQVSSSRRQSCYLCDLPRMPWAMIWDFSEPVCRGCVNYEGADR |
| | 2BP1_2 N- | IEFVIETARQLKRAHGCFQDGRSPGPPPPVGVKTVALSAKEAAAAA |
| | terminal domain | AAAAAAAAAAQQQQQQQQQQQQQQQQQQQQQQQQQLNHVDGSSKPA |
| | | VLAAPSGLERYGLSAAAAAAAAAAAAVEQRSRFEYPPPPVSLGSSS |
| | | HTARLPNGLGGPNGFPKPTPEEGPPELNRQSPNSSSAAASVASRRG |
| | | THGGLVTGLPNPGGGGGPQLTVPPNLLPQTLLNGPASAAVLPPPPP |
| | | HALGSRGPPTPAPPGAPGGPACLGGTPGVSATSSSASSSTSSSVAE |
| | | VGVGAGGKRPGSVSSTDQERELKEKQRNAEALAELSESLRNRAEEW |
| | | ASKPKMVRDTLLTLAGCTPYEVRFKKDHSLLGRVFAFDAVSKPGMD |
| | | YELKLFIEYPTGSGNVYSSASGVAKQMYQDCMKDFGRGLSSGFKYL |
| | | EYEKKHGSGDWRLLGDLLPEAVRFFKEGVPGADMLPQPYLDASCPM |
| | | LPTALVSLSRAPSAPPGTGALPPAAPSGRGAAASLRKRKASPEPPD |
| | | SAEGALKLGEEQQRQQWMANQSEALKLTMSAGGFAAPGHAAGGPPP |
| | | PPPPLGPHSNRTTPPESAPQNGPSPMAALMSVADTLGTAHSPKDGS |
| | | SVHSTTASARRNSSSPVSPASVPGQRRLASRNGDLNLQVAPPPPSA |
| | | HPGMDQVHPQNIPDSPMANSGPLCCTICHERLEDTHFVQCPSVPSH |
| | | KFCFPCSRESIKAQGATGEVYCPSGEKCPLVGSNVPWAFMQGEIAT |
| | | ILAGDVKVKKERDP |
| 107 | HOXA13 | MTASVLLHPRWIEPTVMFLYDNGGGLVADELNKNMEGAAAAAAAAA |
| | (homeodomain) | AAAAAGAGGGGFPHPAAAAAGGNFSVAAAAAAAAAAAANQCRNLMA |
| | | HPAPLAPGAASAYSSAPGEAPPSAAAAAAAAAAAAAAAAAASSSGG |
| | | PGPAGPAGAEAAKQCSPCSAAAQSSSGPAALPYGYFGSGYYPCARM |
| | | GPHPNAIKSCAQPASAAAAAAFADKYMDTAGPAAEEFSSRAKEFAF |
| | | YHQGYAAGPYHHHQPMPGYLDMPVVPGLGGPGESRHEPLGLPMESY |
| | | QPWALPNGWNGQMYCPKEQAQPPHLWKSTLPDVVSHPSDASSYRRG |
| | | RKKRVPYTKVQLKELEREYATNKFITKDKRRRISATTNLSERQVTI |
| | | WFQNRRVKEKKVINKLKTTS |
| 108 | HOXB13 | MEPGNYATLDGAKDIEGLLGAGGGRNLVAHSPLTSHPAAPTLMPAV |
| | (homeodomain) | NYAPLDLPGSAEPPKQCHPCPGVPQGTSPAPVPYGYFGGGYYSCRV |
| | | SRSSLKPCAQAATLAAYPAETPTAGEEYPSRPTEFAFYPGYPGTYQ |
| | | PMASYLDVSVVQTLGAPGEPRHDSLLPVDSYQSWALAGGWNSQMCC |
| | | QGEQNPPGPFWKAAFADSSGQHPPDACAFRRGRKKRIPYSKGQLRE |
| | | LEREYAANKFITKDKRRKISAATSLSERQITIWFQNRRVKEKKVLA |
| | | KVKNSATP |
| 109 | HOXC13 | MTTSLLLHPRWPESLMYVYEDSAAESGIGGGGGGGGGGTGGAGGGC |
| | (homeodomain) | SGASPGKAPSMDGLGSSCPASHCRDLLPHPVLGRPPAPLGAPQGAV |
| | | YTDIPAPEAARQCAPPPAPPTSSSATLGYGYPFGGSYYGCRLSHNV |
| | | NLQQKPCAYHPGDKYPEPSGALPGDDLSSRAKEFAFYPSFASSYQA |
| | | MPGYLDVSVVPGISGHPEPRHDALIPVEGYQHWALSNGWDSQVYCS |
| | | KEQSQSAHLWKSPFPDVVPLQPEVSSYRRGRKKRVPYTKVQLKELE |
| | | KEYAASKFITKEKRRRISATTNLSERQVTIWFQNRRVKEKKVVSKS |
| | | KAPHLHST |
| 110 | HOXA11 | MDFDERGPCSSNMYLPSCTYYVSGPDFSSLPSFLPQTPSSRPMTYS |
| | (homeodomain) | YSSNLPQVQPVREVTFREYAIEPATKWHPRGNLAHCYSAEELVHRD |
| | | CLQAPSAAGVPGDVLAKSSANVYHHPTPAVSSNFYSTVGRNGVLPQ |
| | | AFDQFFETAYGTPENLASSDYPGDKSAEKGPPAATATSAAAAAAAT |
| | | GAPATSSSDSGGGGGCRETAAAAEEKERRRRPESSSSPESSSGHTE |
| | | DKAGGSSGQRTRKKRCPYTKYQIRELEREFFFSVYINKEKRLQLSR |
| | | MLNLTDRQVKIWFQNRRMKEKKINRDRLQYYSANPLL |
| 111 | HOXC11 | MENSVNLGNFCSPSRKERGADFGERGSCASNLYLPSCTYYMPEFST |
| | (homeodomain) | VSSFLPQAPSRQISYPYSAQVPPVREVSYGLEPSGKWHHRNSYSSC |
| | | YAAADELMHRECLPPSTVTEILMKNEGSYGGHHHPSAPHATPAGFY |
| | | SSVNKNSVLPQAFDRFFDNAYCGGGDPPAEPPCSGKGEAKGEPEAP |
| | | PASGLASRAEAGAEAEAEEENTNPSSSGSAHSVAKEPAKGAAPNAP |
| | | RTRKKRCPYSKFQIRELEREFFENVYINKEKRLQLSRMLNLTDRQV |
| | | KIWFQNRRMKEKKLSRDRLQYFSGNPLL |
| 112 | HOXC10 | MTCPRNVTPNSYAEPLAAPGGGERYSRSAGMYMQSGSDENCGVMRG |
| | (homeodomain) | CGLAPSLSKRDEGSSPSLALNTYPSYLSQLDSWGDPKAAYRLEQPV |
| | | GRPLSSCSYPPSVKEENVCCMYSAEKRAKSGPEAALYSHPLPESCL |
| | | GEHEVPVPSYYRASPSYSALDKTPHCSGANDFEAPFEQRASLNPRA |
| | | EHLESPQLGGKVSFPETPKSDSQTPSPNEIKTEQSLAGPKGSPSES |
| | | EKERAKAADSSPDTSDNEAKEEIKAENTTGNWLTAKSGRKKRCPYT |
| | | KHQTLELEKEFLENMYLTRERRLEISKTINLTDRQVKIWFQNRRMK |
| | | LKKMNRENRIRELTSNFNFT |
| 113 | HOXA10 | MSARKGYLLPSPNYPTTMSCSESPAANSFLVDSLISSGRGEAGGGG |
| | (homeodomain) | GGAGGGGGGGYYAHGGVYLPPAADLPYGLQSCGLFPTLGGKRNEAA |
| | | SPGSGGGGGGLGPGAHGYGPSPIDLWLDAPRSCRMEPPDGPPPPPQ |
| | | QQPPPPPQPPQPAPQATSCSFAQNIKEESSYCLYDSADKCPKVSAT |
| | | AAELAPFPRGPPPDGCALGTSSGVPVPGYFRLSQAYGTAKGYGSGG |
| | | GGAQQLGAGPFPAQPPGRGFDLPPALASGSADAARKERALDSPPPP |
| | | TLACGSGGGSQGDEEAHASSSAAEELSPAPSESSKASPEKDSLGNS |
| | | KGENAANWLTAKSGRKKRCPYTKHQTLELEKEFLENMYLTRERRLE |
| | | ISRSVHLTDRQVKIWFQNRRMKLKKMNRENRIRELTANFNES |
| 114 | HOXB9 | MSISGTLSSYYVDSIISHESEDAPPAKFPSGQYASSRQPGHAEHLE |
| | (homeodomain) | FPSCSFQPKAPVFGASWAPLSPHASGSLPSVYHPYIQPQGVPPAES |
| | | RYLRTWLEPAPRGEAAPGQGQAAVKAEPLLGAPGELLKQGTPEYSL |
| | | ETSAGREAVLSNQRPGYGDNKICEGSEDKERPDQTNPSANWLHARS |
| | | SRKKRCPYTKYQTLELEKEFLENMYLTRDRRHEVARLLNLSERQVK |
| | | IWFQNRRMKMKKMNKEQGKE |
| 115 | HOXA9 | MATTGALGNYYVDSFLLGADAADELSVGRYAPGTLGQPPRQAATLA |
| | (homeodomain) | EHPDFSPCSFQSKATVEGASWNPVHAAGANAVPAAVYHHHHHHPYV |
| | | HPQAPVAAAAPDGRYMRSWLEPTPGALSFAGLPSSRPYGIKPEPLS |
| | | ARRGDCPTLDTHTLSLTDYACGSPPVDREKQPSEGAFSENNAENES |
| | | GGDKPPIDPNNPAANWLHARSTRKKRCPYTKHQTLELEKEFLFNMY |
| | | LTRDRRYEVARLLNLTERQVKIWFQNRRMKMKKINKDRAKDE |
| 116 | ZFP28_HUMAN | NKKLEAVGTGIEPKAMSQGLVTFGDVAVDFSQEEWEWLNPIQRNLY |
| | | RKVMLENYRNLASLGLCVSKPDVISSLEQGKEPW |
| 117 | ZN334_HUMAN | KMKKFQIPVSFQDLTVNFTQEEWQQLDPAQRLLYRDVMLENYSNLV |
| | | SVGYHVSKPDVIFKLEQGEEPWIVEEFSNQNYPD |
| 118 | ZN568_HUMAN | CSQESALSEEEEDTTRPLETVTFKDVAVDLTQEEWEQMKPAQRNLY |
| | | RDVMLENYSNLVTVGCQVTKPDVIFKLEQEEEPW |
| 119 | ZN37A_HUMAN | ITSQGSVSFRDVTVGFTQEEWQHLDPAQRTLYRDVMLENYSHLVSV |
| | | GYCIPKPEVILKLEKGEEPWILEEKFPSQSHLEL |
| 120 | ZN181_HUMAN | PQVTFNDVAIDFTHEEWGWLSSAQRDLYKDVMVQNYENLVSVAGLS |
| | | VTKPYVITLLEDGKEPWMMEKKLSKGMIPDWESR |
| 121 | ZN510_HUMAN | PLRFSTLFQEQQKMNISQASVSFKDVTIEFTQEEWQQMAPVQKNLY |
| | | RDVMLENYSNLVSVGYCCFKPEVIFKLEQGEEPW |
| 122 | ZN862_HUMAN | QDPSAEGLSEEVPVVFEELPVVFEDVAVYFTREEWGMLDKRQKELY |
| | | RDVMRMNYELLASLGPAAAKPDLISKLERRAAPW |
| 123 | ZN140_HUMAN | SQGSVTFRDVAIDFSQEEWKWLQPAQRDLYRCVMLENYGHLVSLGL |
| | | SISKPDVVSLLEQGKEPWLGKREVKRDLFSVSES |
| 124 | ZN208_HUMAN | GSLTFRDVAIEFSLEEWQCLDTAQQNLYRNVMLENYRNLVFLGIAA |
| | | FKPDLIIFLEEGKESWNMKRHEMVEESPVICSHF |
| 125 | ZN248_HUMAN | NKSQEQVSFKDVCVDFTQEEWYLLDPAQKILYRDVILENYSNLVSV |
| | | GYCITKPEVIFKIEQGEEPWILEKGFPSQCHPER |
| 126 | ZN571_HUMAN | PHLLVTFRDVAIDFSQEEWECLDPAQRDLYRDVMLENYSNLISLDL |
| | | ESSCVTKKLSPEKEIYEMESLQWENMGKRINHHL |
| 127 | ZN699_HUMAN | EEERKTAELQKNRIQDSVVFEDVAVDFTQEEWALLDLAQRNLYRDV |
| | | MLENFQNLASLGYPLHTPHLISQWEQEEDLQTVK |
| 128 | ZN726_HUMAN | GLLTFRDVAIEFSLEEWQCLDTAQKNLYRNVMLENYRNLAFLGIAV |
| | | SKPDLIICLEKEKEPWNMKRDEMVDEPPGICPHF |
| 129 | ZIK1_HUMAN | RAPTQVTVSPETHMDLTKGCVTFEDIAIYFSQDEWGLLDEAQRLLY |
| | | LEVMLENFALVASLGCGHGTEDEETPSDQNVSVG |
| 130 | ZNF2_HUMAN | AAVSPTTRCQESVTFEDVAVVFTDEEWSRLVPIQRDLYKEVMLENY |
| | | NSIVSLGLPVPQPDVIFQLKRGDKPWMVDLHGSE |
| 131 | Z705F_HUMAN | HSLEKVTFEDVAIDFTQEEWDMMDTSKRKLYRDVMLENISHLVSLG |
| | | YQISKSYIILQLEQGKELWREGRVFLQDQNPDRE |
| 132 | ZNF14_HUMAN | DSVSFEDVAVNFTLEEWALLDSSQKKLYEDVMQETFKNLVCLGKKW |
| | | EDQDIEDDHRNQGKNRRCHMVERLCESRRGSKCG |
| 133 | ZN471_HUMAN | NVEVVKVMPQDLVTFKDVAIDFSQEEWQWMNPAQKRLYRSMMLENY |
| | | QSLVSLGLCISKPYVISLLEQGREPWEMTSEMTR |
| 134 | ZN624_HUMAN | TQPDEDLHLQAEETQLVKESVTFKDVAIDFTLEEWRLMDPTQRNLH |
| | | KDVMLENYRNLVSLGLAVSKPDMISHLENGKGPW |
| 135 | ZNF84_HUMAN | TMLQESFSFDDLSVDFTQKEWQLLDPSQKNLYKDVMLENYSSLVSL |
| | | GYEVMKPDVIFKLEQGEEPWVGDGEIPSSDSPEV |
| 136 | ZNF7_HUMAN | EVVTFGDVAVHFSREEWQCLDPGQRALYREVMLENHSSVAGLAGFL |
| | | VFKPELISRLEQGEEPWVLDLQGAEGTEAPRTSK |
| 137 | ZN891_HUMAN | RNAEEERMIAVFLTTWLQEPMTFKDVAVEFTQEEWMMLDSAQRSLY |
| | | RDVMLENYRNLTSVEYQLYRLTVISPLDQEEIRN |
| 138 | ZN337_HUMAN | GPQGARRQAFLAFGDVTVDFTQKEWRLLSPAQRALYREVTLENYSH |
| | | LVSLGILHSKPELIRRLEQGEVPWGEERRRRPGP |
| 139 | Z705G_HUMAN | HSLKKLTFEDVAIDFTQEEWAMMDTSKRKLYRDVMLENISHLVSLG |
| | | YQISKSYIILQLEQGKELWREGRVFLQDQNPNRE |
| 140 | ZN529_HUMAN | MPEVEFPDQFFTVLTMDHELVTLRDVVINFSQEEWEYLDSAQRNLY |
| | | WDVMMENYSNLLSLDLESRNETKHLSVGKDIIQN |
| 141 | ZN729_HUMAN | PGAPGSLEMGPLTFRDVTIEFSLEEWQCLDTVQQNLYRDVMLENYR |
| | | NLVFLGMAVFKPDLITCLKQGKEPWNMKRHEMVT |
| 142 | ZN419_HUMAN | RDPAQVPVAADLLTDHEEGYVTFEDVAVYFSQEEWRLLDDAQRLLY |
| | | RNVMLENFTLLASLGLASSKTHEITQLESWEEPF |
| 143 | Z705A_HUMAN | HSLKKVTFEDVAIDFTQEEWAMMDTSKRKLYRDVMLENISHLVSLG |
| | | YQISKSYIILQLEQGKELWREGREFLQDQNPDRE |
| 144 | ZNF45_HUMAN | TKSKEAVTFKDVAVVFSEEELQLLDLAQRKLYRDVMLENFRNVVSV |
| | | GHQSTPDGLPQLEREEKLWMMKMATQRDNSSGAK |
| 145 | ZN302_HUMAN | SQVTFSDVAIDFSHEEWACLDSAQRDLYKDVMVQNYENLVSVGLSV |
| | | TKPYVIMLLEDGKEPWMMEKKLSKAYPFPLSHSV |
| 146 | ZN486_HUMAN | PGPLRSLEMESLQFRDVAVEFSLEEWHCLDTAQQNLYRDVMLENYR |
| | | HLVFLGIIVSKPDLITCLEQGIKPLTMKRHEMIA |
| 147 | ZN621_HUMAN | LQTTWPQESVTFEDVAVYFTQNQWASLDPAQRALYGEVMLENYANV |
| | | ASLVAFPFPKPALISHLERGEAPWGPDPWDTEIL |
| 148 | ZN688_HUMAN | APLLAPRPGETRPGCRKPGTVSFADVAVYFSPEEWGCLRPAQRALY |
| | | RDVMQETYGHLGALGFPGPKPALISWMEQESEAW |
| 149 | ZN33A_HUMAN | NKVEQKSQESVSFKDVTVGFTQEEWQHLDPSQRALYRDVMLENYSN |
| | | LVSVGYCVHKPEVIFRLQQGEEPWKQEEEFPSQS |
| 150 | ZN554_HUMAN | CFSQEERMAAGYLPRWSQELVTFEDVSMDFSQEEWELLEPAQKNLY |
| | | REVMLENYRNVVSLEALKNQCTDVGIKEGPLSPA |
| 151 | ZN878_HUMAN | DSVAFEDVAVNFTQEEWALLDPSQKNLYREVMQETLRNLTSIGKKW |
| | | NNQYIEDEHQNPRRNLRRLIGERLSESKESHQHG |
| 152 | ZN772_HUMAN | MGPAQVPMNSEVIVDPIQGQVNFEDVEVYFSQEEWVLLDEAQRLLY |
| | | RDVMLENFALMASLGHTSFMSHIVASLVMGSEPW |
| 153 | ZN224_HUMAN | TTFKEAMTFKDVAVVFTEEELGLLDLAQRKLYRDVMLENFRNLLSV |
| | | GHQAFHRDTFHFLREEKIWMMKTAIQREGNSGDK |
| 154 | ZN184_HUMAN | DSTLLQGGHNLLSSASFQEAVTFKDVIVDFTQEEWKQLDPGQRDLF |
| | | RDVTLENYTHLVSIGLQVSKPDVISQLEQGTEPW |
| 155 | ZN544_HUMAN | EARSMLVPPQASVCFEDVAMAFTQEEWEQLDLAQRTLYREVTLETW |
| | | EHIVSLGLFLSKSDVISQLEQEEDLCRAEQEAPR |
| 156 | ZNF57_HUMAN | DSVVFEDVAVDFTLEEWALLDSAQRDLYRDVMLETFRNLASVDDGT |
| | | QFKANGSVSLQDMYGQEKSKEQTIPNFTGNNSCA |
| 157 | ZN283_HUMAN | EESHGALISSCNSRTMTDGLVTFRDVAIDFSQEEWECLDPAQRDLY |
| | | VDVMLENYSNLVSLDLESKTYETKKIFSENDIFE |
| 158 | ZN549_HUMAN | VITPQIPMVTEEFVKPSQGHVTFEDIAVYFSQEEWGLLDEAQRCLY |
| | | HDVMLENFSLMASVGCLHGIEAEEAPSEQTLSAQ |
| 159 | ZN211_HUMAN | VQLRPQTRMATALRDPASGSVTFEDVAVYFSWEEWDLLDEAQKHLY |
| | | FDVMLENFALTSSLGCWCGVEHEETPSEQRISGE |
| 160 | ZN615_HUMAN | MQAQESLTLEDVAVDFTWEEWQFLSPAQKDLYRDVMLENYSNLVAV |
| | | GYQASKPDALSKLERGEETCTTEDEIYSRICSEI |
| 161 | ZN253_HUMAN | GPLQFRDVAIEFSLEEWHCLDTAQRNLYRDVMLENYRNLVFLGIVV |
| | | SKPDLVTCLEQGKKPLTMERHEMIAKPPVMSSHF |
| 162 | ZN226_HUMAN | NMFKEAVTFKDVAVAFTEEELGLLGPAQRKLYRDVMVENFRNLLSV |
| | | GHPPFKQDVSPIERNEQLWIMTTATRRQGNLGEK |
| 163 | ZN730_HUMAN | GALTFRDVAIEFSLEEWQCLDTEQQNLYRNVMLDNYRNLVFLGIAV |
| | | SKPDLITCLEQEKEPWNLKTHDMVAKPPVICSHI |
| 164 | Z585A_HUMAN | SPQKSSALAPEDHGSSYEGSVSFRDVAIDFSREEWRHLDPSQRNLY |
| | | RDVMLETYSHLLSVGYQVPEAEVVMLEQGKEPWA |
| 165 | ZN732_HUMAN | ELLTFRDVAIEFSPEEWKCLDPAQQNLYRDVMLENYRNLISLGVAI |
| | | SNPDLVIYLEQRKEPYKVKIHETVAKHPAVCSHF |
| 166 | ZN681_HUMAN | EPLKFRDVAIEFSLEEWQCLDTIQQNLYRNVMLENYRNLVFLGIVV |
| | | SKPDLITCLEQEKEPWTRKRHRMVAEPPVICSHF |
| 167 | ZN667_HUMAN | PSARGKSKSKAPITFGDLAIYFSQEEWEWLSPIQKDLYEDVMLENY |
| | | RNLVSLGLSFRRPNVITLLEKGKAPWMVEPVRRR |
| 168 | ZN649_HUMAN | TKAQESLTLEDVAVDFTWEEWQFLSPAQKDLYRDVMLENYSNLVSV |
| | | GYQAGKPDALTKLEQGEPLWTLEDEIHSPAHPEI |
| 169 | ZN470_HUMAN | SQEEVEVAGIKLCKAMSLGSVTFTDVAIDFSQDEWEWLNLAQRSLY |
| | | KKVMLENYRNLVSVGLCISKPDVISLLEQEKDPW |
| 170 | ZN484_HUMAN | TKSLESVSFKDVTVDFSRDEWQQLDLAQKSLYREVMLENYENLISV |
| | | GCQVPKPEVIFSLEQEEPCMLDGEIPSQSRPDGD |
| 171 | ZN431_HUMAN | SGCPGAERNLLVYSYFEKETLTFRDVAIEFSLEEWECLNPAQQNLY |
| | | MNVMLENYKNLVFLGVAVSKQDPVTCLEQEKEPW |
| 172 | ZN382_HUMAN | PLQGSVSFKDVTVDFTQEEWQQLDPAQKALYRDVMLENYCHFVSVG |
| | | FHMAKPDMIRKLEQGEELWTQRIFPSYSYLEEDG |
| 173 | ZN254_HUMAN | PGPPRSLEMGLLTFRDVAIEFSLEEWQHLDIAQQNLYRNVMLENYR |
| | | NLAFLGIAVSKPDLITCLEQGKEPWNMKRHEMVD |
| 174 | ZN124_HUMAN | SGHPGSWEMNSVAFEDVAVNFTQEEWALLDPSQKNLYRDVMQETER |
| | | NLASIGNKGEDQSIEDQYKNSSRNLRHIISHSGN |
| 175 | ZN607_HUMAN | SYGSITFGDVAIDFSHQEWEYLSLVQKTLYQEVMMENYDNLVSLAG |
| | | HSVSKPDLITLLEQGKEPWMIVREETRGECTDLD |
| 176 | ZN317_HUMAN | DLFVCSGLEPHTPSVGSQESVTFQDVAVDFTEKEWPLLDSSQRKLY |
| | | KDVMLENYSNLTSLGYQVGKPSLISHLEQEEEPR |
| 177 | ZN620_HUMAN | FQTAWRQEPVTFEDVAVYFTQNEWASLDSVQRALYREVMLENYANV |
| | | ASLAFPFTTPVLVSQLEQGELPWGLDPWEPMGRE |
| 178 | ZN141_HUMAN | ELLTFRDVAIEFSPEEWKCLDPDQQNLYRDVMLENYRNLVSLGVAI |
| | | SNPDLVTCLEQRKEPYNVKIHKIVARPPAMCSHF |
| 179 | ZN584_HUMAN | AGEAEAQLDPSLQGLVMFEDVTVYFSREEWGLLNVTQKGLYRDVML |
| | | ENFALVSSLGLAPSRSPVFTQLEDDEQSWVPSWV |
| 180 | ZN540_HUMAN | AHALVTFRDVAIDFSQKEWECLDTTQRKLYRDVMLENYNNLVSLGY |
| | | SGSKPDVITLLEQGKEPCVVARDVTGRQCPGLLS |
| 181 | ZN75D_HUMAN | KRIKHWKMASKLILPESLSLLTFEDVAVYFSEEEWQLLNPLEKTLY |
| | | NDVMQDIYETVISLGLKLKNDTGNDHPISVSTSE |
| 182 | ZN555_HUMAN | DSVVFEDVAVDETLEEWALLDSAQRDLYRDVMLETFQNLASVDDET |
| | | QFKASGSVSQQDIYGEKIPKESKIATFTRNVSWA |
| 183 | ZN658_HUMAN | NMSQASVSFQDVTVEFTREEWQHLGPVERTLYRDVMLENYSHLISV |
| | | GYCITKPKVISKLEKGEEPWSLEDEFLNQRYPGY |
| 184 | ZN684_HUMAN | ISFQESVTFQDVAVDFTAEEWQLLDCAERTLYWDVMLENYRNLISV |
| | | GCPITKTKVILKVEQGQEPWMVEGANPHESSPES |
| 185 | RBAK_HUMAN | NTLQGPVSFKDVAVDFTQEEWQQLDPDEKITYRDVMLENYSHLVSV |
| | | GYDTTKPNVIIKLEQGEEPWIMGGEFPCQHSPEA |
| 186 | ZN829_HUMAN | HPEEEERMHDELLQAVSKGPVMFRDVSIDFSQEEWECLDADQMNLY |
| | | KEVMLENFSNLVSVGLSNSKPAVISLLEQGKEPW |
| 187 | ZN582_HUMAN | SLGSELFRDVAIVFSQEEWQWLAPAQRDLYRDVMLETYSNLVSLGL |
| | | AVSKPDVISFLEQGKEPWMVERVVSGGLCPVLES |
| 188 | ZN112_HUMAN | TKFQEMVTFKDVAVVFTEEELGLLDSVQRKLYRDVMLENFRNLLLV |
| | | AHQPFKPDLISQLEREEKLLMVETETPRDGCSGR |
| 189 | ZN716_HUMAN | AKRPGPPGSREMGLLTFRDIAIEFSLAEWQCLDHAQQNLYRDVMLE |
| | | NYRNLVSLGIAVSKPDLITCLEQNKEPQNIKRNE |
| 190 | HKR1_HUMAN | TCMVHRQTMSCSGAGGITAFVAFRDVAVYFTQEEWRLLSPAQRTLH |
| | | REVMLETYNHLVSLEIPSSKPKLIAQLERGEAPW |
| 191 | ZN350_HUMAN | IQAQESITLEDVAVDFTWEEWQLLGAAQKDLYRDVMLENYSNLVAV |
| | | GYQASKPDALFKLEQGEQLWTIEDGIHSGACSDI |
| 192 | ZN480_HUMAN | AQKRRKRKAKESGMALPQGHLTFRDVAIEFSQAEWKCLDPAQRALY |
| | | KDVMLENYRNLVSLGISLPDLNINSMLEQRREPW |
| 193 | ZN416_HUMAN | DSTSVPVTAEAKLMGFTQGCVTFEDVAIYFSQEEWGLLDEAQRLLY |
| | | RDVMLENFALITALVCWHGMEDEETPEQSVSVEG |
| 194 | ZNF92_HUMAN | GPLTFRDVKIEFSLEEWQCLDTAQRNLYRDVMLENYRNLVFLGIAV |
| | | SKPDLITWLEQGKEPWNLKRHEMVDKTPVMCSHF |
| 195 | ZN100_HUMAN | SGCPGAERSLLVQSYFEKGPLTFRDVAIEFSLEEWQCLDSAQQGLY |
| | | RKVMLENYRNLVFLAGIALTKPDLITCLEQGKEP |
| 196 | ZN736_HUMAN | GVLTFRDVAVEFSPEEWECLDSAQQRLYRDVMLENYGNLVSLGLAI |
| | | FKPDLMTCLEQRKEPWKVKRQEAVAKHPAGSFHF |
| 197 | ZNF74_HUMAN | KENLEDISGWGLPEARSKESVSFKDVAVDFTQEEWGQLDSPQRALY |
| | | RDVMLENYQNLLALGPPLHKPDVISHLERGEEPW |
| 198 | CBX1_HUMAN | EESEKPRGFARGLEPERIIGATDSSGELMFLMKWKNSDEADLVPAK |
| | | EANVKCPQVVISFYEERLTWHSYPSEDDDKKDDK |
| 199 | ZN443_HUMAN | ASVALEDVAVNFTREEWALLGPCQKNLYKDVMQETIRNLDCVVMKW |
| | | KDQNIEDQYRYPRKNLRCRMLERFVESKDGTQCG |
| 200 | ZN195_HUMAN | TLLTFRDVAIEFSLEEWKCLDLAQQNLYRDVMLENYRNLFSVGLTV |
| | | CKPGLITCLEQRKEPWNVKRQEAADGHPEMGFHH |
| 201 | ZN530_HUMAN | AAALRAPTQQVFVAFEDVAIYFSQEEWELLDEMQRLLYRDVMLENF |
| | | AVMASLGCWCGAVDEGTPSAESVSVEELSQGRTP |
| 202 | ZN782_HUMAN | NTFQASVSFQDVTVEFSQEEWQHMGPVERTLYRDVMLENYSHLVSV |
| | | GYCFTKPELIFTLEQGEDPWLLEKEKGFLSRNSP |
| 203 | ZN791_HUMAN | DSVAFEDVSVSFSQEEWALLAPSQKKLYRDVMQETFKNLASIGEKW |
| | | EDPNVEDQHKNQGRNLRSHTGERLCEGKEGSQCA |
| 204 | ZN331_HUMAN | AQGLVTFADVAIDFSQEEWACLNSAQRDLYWDVMLENYSNLVSLDL |
| | | ESAYENKSLPTEKNIHEIRASKRNSDRRSKSLGR |
| 205 | Z354C_HUMAN | AVDLLSAQEPVTFRDVAVFFSQDEWLHLDSAQRALYREVMLENYSS |
| | | LVSLGIPFSMPKLIHQLQQGEDPCMVEREVPSDT |
| 206 | ZN157_HUMAN | SPQRFPALIPGEPGRSFEGSVSFEDVAVDFTRQEWHRLDPAQRTMH |
| | | KDVMLETYSNLASVGLCVAKPEMIFKLERGEELW |
| 207 | ZN727_HUMAN | RVLTFRDVAVEFSPEEWECLDSAQQRLYRDVMLENYGNLFSLGLAI |
| | | FKPDLITYLEQRKEPWNARRQKTVAKHPAGSLHF |
| 208 | ZN550_HUMAN | AETKDAAQMLVTFKDVAVTFTREEWRQLDLAQRTLYREVMLETCGL |
| | | LVSLGHRVPKPELVHLLEHGQELWIVKRGLSHAT |
| 209 | ZN793_HUMAN | IEYQIPVSFKDVVVGFTQEEWHRLSPAQRALYRDVMLETYSNLVSV |
| | | GYEGTKPDVILRLEQEEAPWIGEAACPGCHCWED |
| 210 | ZN235_HUMAN | TKFQEAVTFKDVAVAFTEEELGLLDSAQRKLYRDVMLENFRNLVSV |
| | | GHQSFKPDMISQLEREEKLWMKELQTQRGKHSGD |
| 211 | ZNF8_HUMAN | DEGVAGVMSVGPPAARLQEPVTFRDVAVDFTQEEWGQLDPTQRILY |
| | | RDVMLETFGHLLSIGPELPKPEVISQLEQGTELW |
| 212 | ZN724_HUMAN | GPLTFMDVAIEFSVEEWQCLDTAQQNLYRNVMLENYRNLVFLGIAV |
| | | SKPDLITCLEQGKEPWNMERHEMVAKPPGMCCYF |
| 213 | ZN573_HUMAN | HQVGLIRSYNSKTMTCFQELVTFRDVAIDFSRQEWEYLDPNQRDLY |
| | | RDVMLENYRNLVSLGGHSISKPVVVDLLERGKEP |
| 214 | ZN577_HUMAN | NATIVMSVRREQGSSSGEGSLSFEDVAVGFTREEWQFLDQSQKVLY |
| | | KEVMLENYINLVSIGYRGTKPDSLFKLEQGEPPG |
| 215 | ZN789_HUMAN | FPPARGKELLSFEDVAMYFTREEWGHLNWGQKDLYRDVMLENYRNM |
| | | VLLGFQFPKPEMICQLENWDEQWILDLPRTGNRK |
| 216 | ZN718_HUMAN | ELLTFKDVAIEFSPEEWKCLDTSQQNLYRDVMLENYRNLVSLGVSI |
| | | SNPDLVTSLEQRKEPYNLKIHETAARPPAVCSHF |
| 217 | ZN300_HUMAN | MKSQGLVSFKDVAVDFTQEEWQQLDPSQRTLYRDVMLENYSHLVSM |
| | | GYPVSKPDVISKLEQGEEPWIIKGDISNWIYPDE |
| 218 | ZN383_HUMAN | AEGSVMFSDVSIDFSQEEWDCLDPVQRDLYRDVMLENYGNLVSMGL |
| | | YTPKPQVISLLEQGKEPWMVGRELTRGLCSDLES |
| 219 | ZN429_HUMAN | GPLTFTDVAIEFSLEEWQCLDTAQQNLYRNVMLENYRNLVFLGIAV |
| | | SKPDLITCLEKEKEPCKMKRHEMVDEPPVVCSHF |
| 220 | ZN677_HUMAN | ALSQGLFTFKDVAIEFSQEEWECLDPAQRALYRDVMLENYRNLLSL |
| | | DEDNIPPEDDISVGFTSKGLSPKENNKEELYHLV |
| 221 | ZN850_HUMAN | NMEGLVMFQDLSIDFSQEEWECLDAAQKDLYRDVMMENYSSLVSLG |
| | | LSIPKPDVISLLEQGKEPWMVSRDVLGGWCRDSE |
| 222 | ZN454_HUMAN | AVSHLPTMVQESVTFKDVAILFTQEEWGQLSPAQRALYRDVMLENY |
| | | SNLVSLGLLGPKPDTFSQLEKREVWMPEDTPGGF |
| 223 | ZN257_HUMAN | GPLTIRDVTVEFSLEEWHCLDTAQQNLYRDVMLENYRNLVFLGIAV |
| | | SKPDLITCLEQGKEPCNMKRHEMVAKPPVMCSHI |
| 224 | ZN264_HUMAN | AAAVLTDRAQVSVTFDDVAVTFTKEEWGQLDLAQRTLYQEVMLENC |
| | | GLLVSLGCPVPKAELICHLEHGQEPWTRKEDLSQ |
| 225 | ZFP82_HUMAN | ALRSVMFSDVSIDFSPEEWEYLDLEQKDLYRDVMLENYSNLVSLGC |
| | | FISKPDVISSLEQGKEPWKVVRKGRRQYPDLETK |
| 226 | ZFP14_HUMAN | AHGSVTFRDVAIDFSQEEWEFLDPAQRDLYRDVMWENYSNFISLGP |
| | | SISKPDVITLLDEERKEPGMVVREGTRRYCPDLE |
| 227 | ZN485_HUMAN | APRAQIQGPLTFGDVAVAFTRIEWRHLDAAQRALYRDVMLENYGNL |
| | | VSVGLLSSKPKLITQLEQGAEPWTEVREAPSGTH |
| 228 | ZN737_HUMAN | GPLQFRDVAIEFSLEEWHCLDTAQRNLYRNVMLENYRNLVFLGIVV |
| | | SKPDLITCLEQGKKPLTMKKHEMVANPSVTCSHF |
| 229 | ZNF44_HUMAN | TLPRGQPEVLEWGLPKDQDSVAFEDVAVNFTHEEWALLGPSQKNLY |
| | | RDVMRETIRNLNCIGMKWENQNIDDQHQNLRRNP |
| 230 | ZN596_HUMAN | PSPDSMTFEDIIVDFTQEEWALLDTSQRKLFQDVMLENISHLVSIG |
| | | KQLCKSVVLSQLEQVEKLSTQRISLLQGREVGIK |
| 231 | ZN565_HUMAN | EESREIRAGQIVLKAMAQGLVTFRDVAIEFSLEEWKCLEPAQRDLY |
| | | REVTLENFGHLASLGLSISKPDVVSLLEQGKEPW |
| 232 | ZN543_HUMAN | AASAQVSVTFEDVAVTFTQEEWGQLDAAQRTLYQEVMLETCGLLMS |
| | | LGCPLFKPELIYQLDHRQELWMATKDLSQSSYPG |
| 233 | ZFP69_HUMAN | RESLEDEVTPGLPTAESQELLTFKDISIDFTQEEWGQLAPAHQNLY |
| | | REVMLENYSNLVSVGYQLSKPSVISQLEKGEEPW |
| 234 | SUMO1_HUMAN | EGEYIKLKVIGQDSSEIHFKVKMTTHLKKLKESYCQRQGVPMNSLR |
| | | FLFEGQRIADNHTPKELGMEEEDVIEVYQEQTGG |
| 235 | ZNF12_HUMAN | NKSLGPVSFKDVAVDFTQEEWQQLDPEQKITYRDVMLENYSNLVSV |
| | | GYHIIKPDVISKLEQGEEPWIVEGEFLLQSYPDE |
| 236 | ZN169_HUMAN | SPGLLTTRKEALMAFRDVAVAFTQKEWKLLSSAQRTLYREVMLENY |
| | | SHLVSLGIAFSKPKLIEQLEQGDEPWREENEHLL |
| 237 | ZN433_HUMAN | MFQDSVAFEDVAVTFTQEEWALLDPSQKNLCRDVMQETERNLASIG |
| | | KKWKPQNIYVEYENLRRNLRIVGERLFESKEGHQ |
| 238 | SUMO3_HUMAN | ENDHINLKVAGQDGSVVQFKIKRHTPLSKLMKAYCERQGLSMRQIR |
| | | FRFDGQPINETDTPAQLEMEDEDTIDVEQQQTGG |
| 239 | ZNF98_HUMAN | PGPLGSLEMGVLTFRDVALEFSLEEWQCLDTAQQNLYRNVMLENYR |
| | | NLVFVGIAASKPDLITCLEQGKEPWNVKRHEMVT |
| 240 | ZN175_HUMAN | LSQKPQVLGPEKQDGSCEASVSFEDVTVDFSREEWQQLDPAQRCLY |
| | | RDVMLELYSHLFAVGYHIPNPEVIFRMLKEKEPR |
| 241 | ZN347_HUMAN | ALTQGQVTFRDVAIEFSQEEWTCLDPAQRTLYRDVMLENYRNLASL |
| | | GISCFDLSIISMLEQGKEPFTLESQVQIAGNPDG |
| 242 | ZNF25_HUMAN | NKFQGPVTLKDVIVEFTKEEWKLLTPAQRTLYKDVMLENYSHLVSV |
| | | GYHVNKPNAVFKLKQGKEPWILEVEFPHRGFPED |
| 243 | ZN519_HUMAN | ELLTFRDVAIEFSPEEWKCLDPAQQNLYRDVMLENYRNLVSLAVYS |
| | | YYNQGILPEQGIQDSFKKATLGRYGSCGLENICL |
| 244 | Z585B_HUMAN | SPQKSSALAPEDHGSSYEGSVSFRDVAIDFSREEWRHLDLSQRNLY |
| | | RDVMLETYSHLLSVGYQVPKPEVVMLEQGKEPWA |
| 245 | ZIM3_HUMAN | NNSQGRVTFEDVTVNFTQGEWQRLNPEQRNLYRDVMLENYSNLVSV |
| | | GQGETTKPDVILRLEQGKEPWLEEEEVLGSGRAE |
| 246 | ZN517_HUMAN | AMALPMPGPQEAVVFEDVAVYFTRIEWSCLAPDQQALYRDVMLENY |
| | | GNLASLGELVAKPALISLLEQGEEPGALILQVAE |
| 247 | ZN846_HUMAN | DSSQHLVTFEDVAVDFTQEEWTLLDQAQRDLYRDVMLENYKNLIIL |
| | | AGSELFKRSLMSGLEQMEELRTGVTGVLQELDLQ |
| 248 | ZN230_HUMAN | TTFKEAVTFKDVAVFFTEEELGLLDPAQRKLYQDVMLENFINLLSV |
| | | GHQPFHPFHFLREEKFWMMETATQREGNSGGKTI |
| 249 | ZNF66_HUMAN | GPLQFRDVAIEFSLEEWHCLDMAQRNLYRDVMLENYRNLVFLGIVV |
| | | SKPDLITHLEQGKKPSTMQRHEMVANPSVLCSHF |
| 250 | ZFP1_HUMAN | NKSQGSVSFTDVTVDFTQEEWEQLDPSQRILYMDVMLENYSNLLSV |
| | | EVWKADDQMERDHRNPDEQARQFLILKNQTPIEE |
| 251 | ZN713_HUMAN | EEEEMNDGSQMVRSQESLTFQDVAVDFTREEWDQLYPAQKNLYRDV |
| | | MLENYRNLVALGYQLCKPEVIAQLELEEEWVIER |
| 252 | ZN816_HUMAN | EEATKKSKEKEPGMALPQGRLTFRDVAIEFSLEEWKCLNPAQRALY |
| | | RAVMLENYRNLEFVDSSLKSMMEFSSTRHSITGE |
| 253 | ZN426_HUMAN | EKTPAGRIVADCLTDCYQDSVTFDDVAVDFTQEEWTLLDSTQRSLY |
| | | SDVMLENYKNLATVGGQIIKPSLISWLEQEESRT |
| 254 | ZN674_HUMAN | AMSQESLTFKDVFVDFTLEEWQQLDSAQKNLYRDVMLENYSHLVSV |
| | | GHLVGKPDVIFRLGPGDESWMADGGTPVRTCAGE |
| 255 | ZN627_HUMAN | DSVAFEDVAVNFTLEEWALLDPSQKNLYRDVMRETFRNLASVGKQW |
| | | EDQNIEDPFKIPRRNISHIPERLCESKEGGQGEE |
| 256 | ZNF20_HUMAN | MFQDSVAFEDVAVSFTQEEWALLDPSQKNLYRDVMQETFKNLTSVG |
| | | KTWKVQNIEDEYKNPRRNLSLMREKLCESKESHH |
| 257 | Z587B_HUMAN | AVVATLRLSAQGTVTFEDVAVKFTQEEWNLLSEAQRCLYRDVTLEN |
| | | LALMSSLGCWCGVEDEAAPSKQSIYIQRETQVRT |
| 258 | ZN316_HUMAN | EEEEEDEDEDDLLTAGCQELVTFEDVAVYFSLEEWERLEADQRGLY |
| | | QEVMQENYGILVSLGYPIPKPDLIFRLEQGEEPW |
| 259 | ZN233_HUMAN | TKFQEMVTFKDVAVVFTREELGLLDLAQRKLYQDVMLENFRNLLSV |
| | | GYQPFKLDVILQLGKEDKLRMMETEIQGDGCSGH |
| 260 | ZN611_HUMAN | EEAAQKRKGKEPGMALPQGRLTFRDVAIEFSLAEWKCLNPSQRALY |
| | | REVMLENYRNLEAVDISSKCMMKEVLSTGQGNTE |
| 261 | ZN556_HUMAN | DTVVFEDVVVDFTLEEWALLNPAQRKLYRDVMLETFKHLASVDNEA |
| | | QLKASGSISQQDTSGEKLSLKQKIEKFTRKNIWA |
| 262 | ZN234_HUMAN | TTFKEGLTFKDVAVVFTEEELGLLDPVQRNLYQDVMLENFRNLLSV |
| | | GHHPFKHDVFLLEKEKKLDIMKTATQRKGKSADK |
| 263 | ZN560_HUMAN | SALQQEFWKIQTSNGIQMDLVTFDSVAVEFTQEEWTLLDPAQRNLY |
| | | SDVMLENYKNLSSVGYQLFKPSLISWLEEEEELS |
| 264 | ZNF77_HUMAN | DCVIFEEVAVNETPEEWALLDHAQRSLYRDVMLETCRNLASLDCYI |
| | | YVRTSGSSSQRDVFGNGISNDEEIVKFTGSDSWS |
| 265 | ZN682_HUMAN | ELLTFRDVTIEFSLEEWEFLNPAQQSLYRKVMLENYRNLVSLGLTV |
| | | SKPELISRLEQRQEPWNVKRHETIAKPPAMSSHY |
| 266 | ZN614_HUMAN | IKTQESLTLEDVAVEFSWEEWQLLDTAQKNLYRDVMVENYNHLVSL |
| | | GYQTSKPDVLSKLAHGQEPWTTDAKIQNKNCPGI |
| 267 | ZN785_HUMAN | PAHVPGEAGPRRTRESRPGAVSFADVAVYFSPEEWECLRPAQRALY |
| | | RDVMRETFGHLGALGFSVPKPAFISWVEGEVEAW |
| 268 | ZN445_HUMAN | GCPGDQVTPTRSLTAQLQETMTFKDVEVTFSQDEWGWLDSAQRNLY |
| | | RDVMLENYRNMASLVGPFTKPALISWLEAREPWG |
| 269 | ZFP30_HUMAN | ARDLVMFRDVAVDFSQEEWECLNSYQRNLYRDVILENYSNLVSLAG |
| | | CSISKPDVITLLEQGKEPWMVVRDEKRRWILDLE |
| 270 | ZN225_HUMAN | TTLKEAVTFKDVAVVFTEEELRLLDLAQRKLYREVMLENFRNLLSV |
| | | GHQSLHRDTFHFLKEEKFWMMETATQREGNLGGK |
| 271 | ZN551_HUMAN | SPPSPRSSMAAVALRDSAQGMTFEDVAIYFSQEEWELLDESQRFLY |
| | | CDVMLENFAHVTSLGYCHGMENEAIASEQSVSIQ |
| 272 | ZN610_HUMAN | DEEAQKRKAKESGMALPQGRLTFMDVAIEFSQEEWKSLDPGQRALY |
| | | RDVMLENYRNLVFLGICLPDLSIISMLKQRREPL |
| 273 | ZN528_HUMAN | ALTQGPLKFMDVAIEFSQEEWKCLDPAQRTLYRDVMLENYRNLVSL |
| | | GICLPDLSVTSMLEQKRDPWTLQSEEKIANDPDG |
| 274 | ZN284_HUMAN | TMFKEAVTFKDVAVVFTEEELGLLDVSQRKLYRDVMLENFRNLLSV |
| | | GHQLSHRDTFHFQREEKFWIMETATQREGNSGGK |
| 275 | ZN418_HUMAN | QGTVAFEDVAVNFSQEEWSLLSEVQRCLYHDVMLENWVLISSLGCW |
| | | CGSEDEEAPSKKSISIQRVSQVSTPGAGVSPKKA |
| 276 | MPP8_HUMAN | AEAFGDSEEDGEDVFEVEKILDMKTEGGKVLYKVRWKGYTSDDDTW |
| | | EPEIHLEDCKEVLLEFRKKIAENKAKAVRKDIQR |
| 277 | ZN490_HUMAN | VLQMQNSEHHGQSIKTQTDSISLEDVAVNFTLEEWALLDPGQRNIY |
| | | RDVMRATFKNLACIGEKWKDQDIEDEHKNQGRNL |
| 278 | ZN805_HUMAN | AMALTDPAQVSVTFDDVAVTFTQEEWGQLDLAQRTLYQEVMLENCG |
| | | LLVSLGCPVPRPELIYHLEHGQEPWTRKEDLSQG |
| 279 | Z780B_HUMAN | VHGSVTFRDVAIDFSQEEWECLQPDQRTLYRDVMLENYSHLISLGS |
| | | SISKPDVITLLEQEKEPWIVVSKETSRWYPDLES |
| 280 | ZN763_HUMAN | DPVACEDVAVNFTQEEWALLDISQRKLYREVMLETFRNLTSIGKKW |
| | | KDQNIEYEYQNPRRNFRSLIEGNVNEIKEDSHCG |
| 281 | ZN285_HUMAN | IKFQERVTFKDVAVVFTKEELALLDKAQINLYQDVMLENFRNLMLV |
| | | RDGIKNNILNLQAKGLSYLSQEVLHCWQIWKQRI |
| 282 | ZNF85_HUMAN | GPLTFRDVAIEFSLKEWQCLDTAQRNLYRNVMLENYRNLVFLGITV |
| | | SKPDLITCLEQGKEAWSMKRHEIMVAKPTVMCSH |
| 283 | ZN223_HUMAN | TMSKEAVTFKDVAVVFTEEELGLLDLAQRKLYRDVMLENFRNLLSV |
| | | GHQPFHRDTFHFLREEKFWMMDIATQREGNSGGK |
| 284 | ZNF90_HUMAN | GPLEFRDVAIEFSLEEWHCLDTAQQNLYRDVMLENYRHLVFLGIVV |
| | | TKPDLITCLEQGKKPFTVKRHEMIAKSPVMCFHF |
| 285 | ZN557_HUMAN | GHTEGGELVNELLKSWLKGLVTFEDVAVEFTQEEWALLDPAQRTLY |
| | | RDVMLENCRNLASLGNQVDKPRLISQLEQEDKVM |
| 286 | ZN425_HUMAN | AEPASVTVTEDDVALYFSEQEWEILEKWQKQMYKQEMKTNYETLDS |
| | | LGYAFSKPDLITWMEQGRMLLISEQGCLDKTRRT |
| 287 | ZN229_HUMAN | HSQASAISQDREEKIMSQEPLSFKDVAVVFTEEELELLDSTQRQLY |
| | | QDVMQENFRNLLSVGERNPLGDKNGKDTEYIQDE |
| 288 | ZN606_HUMAN | GSLEEGRRATGLPAAQVQEPVTFKDVAVDFTQEEWGQLDLVQRTLY |
| | | RDVMLETYGHLLSVGNQIAKPEVISLLEQGEEPW |
| 289 | ZN155_HUMAN | TTFKEAVTFKDVAVVFTEEELGLLDPAQRKLYRDVMLENFRNLLSV |
| | | GHQPFHQDTCHFLREEKFWMMGTATQREGNSGGK |
| 290 | ZN222_HUMAN | AKLYEAVTFKDVAVIFTEEELGLLDPAQRKLYRDVMLENFRNLLSV |
| | | GGKIQTEMETVPEAGTHEEFSCKQIWEQIASDLT |
| 291 | ZN442_HUMAN | RSDLFLPDSQTNEERKQYDSVAFEDVAVNFTQEEWALLGPSQKSLY |
| | | RDVMWETIRNLDCIGMKWEDTNIEDQHRNPRRSL |
| 292 | ZNF91_HUMAN | PGTPGSLEMGLLTFRDVAIEFSPEEWQCLDTAQQNLYRNVMLENYR |
| | | NLAFLGIALSKPDLITYLEQGKEPWNMKQHEMVD |
| 293 | ZN135_HUMAN | TPGVRVSTDPEQVTFEDVVVGFSQEEWGQLKPAQRTLYRDVMLDTF |
| | | RLLVSVGHWLPKPNVISLLEQEAELWAVESRLPQ |
| 294 | ZN778_HUMAN | EQTQAAGMVAGWLINCYQDAVTEDDVAVDFTQEEWTLLDPSQRDLY |
| | | RDVMLENYENLASVEWRLKTKGPALRQDRSWFRA |
| 295 | RYBP_HUMAN | PSEANSIQSANATTKTSETNHTSRPRLKNVDRSTAQQLAVTVGNVT |
| | | VIITDFKEKTRSSSTSSSTVTSSAGSEQQNQSSS |
| 296 | ZN534_HUMAN | ALTQGQLSFSDVAIEFSQEEWKCLDPGQKALYRDVMLENYRNLVSL |
| | | GEDNVRPEACICSGICLPDLSVTSMLEQKRDPWT |
| 297 | ZN586_HUMAN | AAAAALRAPAQSSVTFEDVAVNFSLEEWSLLNEAQRCLYRDVMLET |
| | | LTLISSLGCWHGGEDEAAPSKQSTCIHIYKDQGG |
| 298 | ZN567_HUMAN | AQGSVSFNDVTVDFTQEEWQHLDHAQKTLYMDVMLENYCHLISVGC |
| | | HMTKPDVILKLERGEEPWTSFAGHTCLEENWKAE |
| 299 | ZN440_HUMAN | DPVAFKDVAVNFTQEEWALLDISQRKLYREVMLETFRNLTSLGKRW |
| | | KDQNIEYEHQNPRRNFRSLIEEKVNEIKDDSHCG |
| 300 | ZN583_HUMAN | SKDLVTFGDVAVNFSQEEWEWLNPAQRNLYRKVMLENYRSLVSLGV |
| | | SVSKPDVISLLEQGKEPWMVKKEGTRGPCPDWEY |
| 301 | ZN441_HUMAN | DSVAFEDVAINFTCEEWALLGPSQKSLYRDVMQETIRNLDCIGMIW |
| | | QNHDIEEDQYKDLRRNLRCHMVERACEIKDNSQC |
| 302 | ZNF43_HUMAN | GPLTFMDVAIEFCLEEWQCLDIAQQNLYRNVMLENYRNLVFLGIAV |
| | | SKPDLITCLEQEKEPWEPMRRHEMVAKPPVMCSH |
| 303 | CBX5_HUMAN | QSNDIARGFERGLEPEKIIGATDSCGDLMFLMKWKDTDEADLVLAK |
| | | EANVKCPQIVIAFYEERLTWHAYPEDAENKEKET |
| 304 | ZN589_HUMAN | ALPAKDSAWPWEEKPRYLGPVTFEDVAVLFTEAEWKRLSLEQRNLY |
| | | KEVMLENLRNLVSLAESKPEVHTCPSCPLAFGSQ |
| 305 | ZNF10_HUMAN | DAKSLTAWSRTLVTFKDVFVDFTREEWKLLDTAQQIVYRNVMLENY |
| | | KNLVSLGYQLTKPDVILRLEKGEEPWLVEREIHQ |
| 306 | ZN563_HUMAN | DAVAFEDVAVNETQEEWALLGPSQKNLYRYVMQETIRNLDCIRMIW |
| | | EEQNTEDQYKNPRRNLRCHMVERFSESKDSSQCG |
| 307 | ZN561_HUMAN | EKTKVERMVEDYLASGYQDSVTFDDVAVDFTPEEWALLDTTEKYLY |
| | | RDVMLENYMNLASVEWEIQPRTKRSSLQQGFLKN |
| 308 | ZN136_HUMAN | DSVAFEDVDVNFTQEEWALLDPSQKNLYRDVMWETMRNLASIGKKW |
| | | KDQNIKDHYKHRGRNLRSHMLERLYQTKDGSQRG |
| 309 | ZN630_HUMAN | IESQEPVTFEDVAVDFTQEEWQQLNPAQKTLHRDVMLETYNHLVSV |
| | | GCSGIKPDVIFKLEHGKDPWIIESELSRWIYPDR |
| 310 | ZN527_HUMAN | AVGLCKAMSQGLVTFRDVALDFSQEEWEWLKPSQKDLYRDVMLENY |
| | | RNLVWLGLSISKPNMISLLEQGKEPWMVERKMSQ |
| 311 | ZN333_HUMAN | DKVEEEAMAPGLPTACSQEPVTFADVAVVFTPEEWVELDSTQRSLY |
| | | RDVMLENYRNLASVADQLCKPNALSYLEERGEQW |
| 312 | Z324B_HUMAN | TFEDVAVYFSQEEWGLLDTAQRALYRHVMLENFTLVTSLGLSTSRP |
| | | RVVIQLERGEEPWVPSGKDMTLARNTYGRLNSGS |
| 313 | ZN786_HUMAN | AEPPRLPLTFEDVAIYFSEQEWQDLEAWQKELYKHVMRSNYETLVS |
| | | LDDGLPKPELISWIEHGGEPFRKWRESQKSGNII |
| 314 | ZN709_HUMAN | DSVVFEDVAVNFTQEEWALLGPSQKKLYRDVMQETFVNLASIGENW |
| | | EEKNIEDHKNQGRKLRSHMVERLCERKEGSQFGE |
| 315 | ZN792_HUMAN | AAAALRDPAQGCVTFEDVTIYFSQEEWVLLDEAQRLLYCDVMLENF |
| | | ALIASLGLISFRSHIVSQLEMGKEPWVPDSVDMT |
| 316 | ZN599_HUMAN | AAPALALVSFEDVVVTFTGEEWGHLDLAQRTLYQEVMLETCRLLVS |
| | | LGHPVPKPELIYLLEHGQELWTVKRGLSQSTCAG |
| 317 | ZN613_HUMAN | IKSQESLTLEDVAVEFTWEEWQLLGPAQKDLYRDVMLENYSNLVSV |
| | | GYQASKPDALFKLEQGEPWTVENEIHSQICPEIK |
| 318 | ZF69B_HUMAN | GESLESRVTLGSLTAESQELLTFKDVSVDFTQEEWGQLAPAHRNLY |
| | | REVMLENYGNLVSVGCQLSKPGVISQLEKGEEPW |
| 319 | ZN799_HUMAN | ASVALEDVAVNFTREEWALLGPCQKNLYKDVMQETIRNLDCVGMKW |
| | | KDQNIEDQYRYPRKNLRCRMLERFVESKDGTQCG |
| 320 | ZN569_HUMAN | TESQGTVTFKDVAIDFTQEEWKRLDPAQRKLYRNVMLENYNNLITV |
| | | GYPFTKPDVIFKLEQEEEPWVMEEEVLRRHWQGE |
| 321 | ZN564_HUMAN | DSVASEDVAVNETLEEWALLDPSQKKLYRDVMRETERNLACVGKKW |
| | | EDQSIEDWYKNQGRILRNHMEEGLSESKEYDQCG |
| 322 | ZN546_HUMAN | EETQGELTSSCGSKTMANVSLAFRDVSIDLSQEEWECLDAVQRDLY |
| | | KDVMLENYSNLVSLGYTIPKPDVITLLEQEKEPW |
| 323 | ZFP92_HUMAN | AAILLTTRPKVPVSFEDVSVYFTKTEWKLLDLRQKVLYKRVMLENY |
| | | SHLVSLGFSFSKPHLISQLERGEGPWVADIPRTW |
| 324 | YAF2_HUMAN | KDKVEKEKSEKETTSKKNSHKKTRPRLKNVDRSSAQHLEVTVGDLT |
| | | VIITDFKEKTKSPPASSAASADQHSQSGSSSDNT |
| 325 | ZN723_HUMAN | GPLTFTDVAIKFSLEEWQFLDTAQQNLYRDVMLENYRNLVFLGVGV |
| | | SKPDLITCLEQGKEPWNMKRHKMVAKPPVVCSHF |
| 326 | ZNF34_HUMAN | RKPNPQAMAALFLSAPPQAEVTFEDVAVYLSREEWGRLGPAQRGLY |
| | | RDVMLETYGNLVSLGVGPAGPKPGVISQLERGDE |
| 327 | ZN439_HUMAN | LSLSPILLYTCEMFQDPVAFKDVAVNETQEEWALLDISQKNLYREV |
| | | MLETFWNLTSIGKKWKDQNIEYEYQNPRRNFRSV |
| 328 | ZFP57_HUMAN | AAGEPRSLLFFQKPVTFEDVAVNFTQEEWDCLDASQRVLYQDVMSE |
| | | TFKNLTSVARIFLHKPELITKLEQEEEQWRETRV |
| 329 | ZNF19_HUMAN | AAMPLKAQYQEMVTFEDVAVHFTKTEWTGLSPAQRALYRSVMLENF |
| | | GNLTALGYPVPKPALISLLERGDMAWGLEAQDDP |
| 330 | ZN404_HUMAN | ARVPLTFSDVAIDFSQEEWEYLNSDQRDLYRDVMLENYTNLVSLDF |
| | | NETTESNKLSSEKRNYEVNAYHQETWKRNKTENL |
| 331 | ZN274_HUMAN | ASRLPTAWSCEPVTFEDVTLGFTPEEWGLLDLKQKSLYREVMLENY |
| | | RNLVSVEHQLSKPDVVSQLEEAEDFWPVERGIPQ |
| 332 | CBX3_HUMAN | SKKKRDAADKPRGFARGLDPERIIGATDSSGELMFLMKWKDSDEAD |
| | | LVLAKEANMKCPQIVIAFYEERLTWHSCPEDEAQ |
| 333 | ZNF30_HUMAN | AHKYVGLQYHGSVTFEDVAIAFSQQEWESLDSSQRGLYRDVMLENY |
| | | RNLVSMGHSRSKPHVIALLEQWKEPEVTVRKDGR |
| 334 | ZN250_HUMAN | AAARLLPVPAGPQPLSFQAKLTFEDVAVLLSQDEWDRLCPAQRGLY |
| | | RNVMMETYGNVVSLGLPGSKPDIISQLERGEDPW |
| 335 | ZN570_HUMAN | AVGLLKAMYQELVTFRDVAVDFSQEEWDCLDSSQRHLYSNVMLENY |
| | | RILVSLGLCFSKPSVILLLEQGKAPWMVKRELTK |
| 336 | ZN675_HUMAN | GLLTFRDVAIEFSLEEWQCLDTAQRNLYKNVILENYRNLVFLGIAV |
| | | SKQDLITCLEQEKEPLTVKRHEMVNEPPVMCSHF |
| 337 | ZN695_HUMAN | GLLAFRDVALEFSPEEWECLDPAQRSLYRDVMLENYRNLISLGEDS |
| | | FNMQFLFHSLAMSKPELIICLEARKEPWNVNTEK |
| 338 | ZN548_HUMAN | NLTEGRVVFEDVAIYFSQEEWGHLDEAQRLLYRDVMLENLALLSSL |
| | | GSWHGAEDEEAPSQQGFSVGVSEVTASKPCLSSQ |
| 339 | ZN132_HUMAN | GPAQHTSWPCGSAVPTLKSMVTFEDVAVYFSQEEWELLDAAQRHLY |
| | | HSVMLENLELVTSLGSWHGVEGEGAHPKQNVSVE |
| 340 | ZN738_HUMAN | SGYPGAERNLLEYSYFEKGPLTFRDVVIEFSQEEWQCLDTAQQDLY |
| | | RKVMLENFRNLVFLGIDVSKPDLITCLEQGKDPW |
| 341 | ZN420_HUMAN | ARKLVMFRDVAIDFSQEEWECLDSAQRDLYRDVMLENYSNLVSLDL |
| | | PSRCASKDLSPEKNTYETELSQWEMSDRLENCDL |
| 342 | ZN626_HUMAN | GPLQFRDVAIEFSLEEWHCLDTAQRNLYRNVMLENYSNLVELGITV |
| | | SKPDLITCLEQGRKPLTMKRNEMIAKPSVMCSHF |
| 343 | ZN559_HUMAN | VAGWLTNYSQDSVTFEDVAVDFTQEEWILLDQTQRNLYRDVMLENY |
| | | KNLVAVDWESHINTKWSAPQQNFLQGKTSSVVEM |
| 344 | ZN460_HUMAN | AAAWMAPAQESVTFEDVAVTFTQEEWGQLDVTQRALYVEVMLETCG |
| | | LLVALGDSTKPETVEPIPSHLALPEEVSLQEQLA |
| 345 | ZN268_HUMAN | VLEWLFISQEQPKITKSWGPLSFMDVFVDFTWEEWQLLDPAQKCLY |
| | | RSVMLENYSNLVSLGYQHTKPDIIFKLEQGEELC |
| 346 | ZN304_HUMAN | AAAVLMDRVQSCVTFEDVFVYFSREEWELLEEAQRFLYRDVMLENF |
| | | ALVATLGFWCEAEHEAPSEQSVSVEGVSQVRTAE |
| 347 | ZIM2_HUMAN | AGSQFPDFKHLGTFLVFEELVTFEDVLVDFSPEELSSLSAAQRNLY |
| | | REVMLENYRNLVSLGHQFSKPDIISRLEEEESYA |
| 348 | ZN605_HUMAN | IQSQISFEDVAVDFTLEEWQLLNPTQKNLYRDVMLENYSNLVFLEV |
| | | WLDNPKMWLRDNQDNLKSMERGHKYDVFGKIFNS |
| 349 | ZN844_HUMAN | DLVAFEDVAVNFTQEEWSLLDPSQKNLYREVMQETLRNLASIGEKW |
| | | KDQNIEDQYKNPRNNLRSLLGERVDENTEENHCG |
| 350 | SUMO5_HUMAN | KDEDIKLRVIGQDSSEIHFKVKMTTPLKKLKKSYCQRQGVPVNSLR |
| | | FLFEGQRIADNHTPEELGMEEEDVIEVYQEQIGG |
| 351 | ZN101_HUMAN | DSVAFEDVAVNFTQEEWALLSPSQKNLYRDVTLETFRNLASVGIQW |
| | | KDQDIENLYQNLGIKLRSLVERLCGRKEGNEHRE |
| 352 | ZN783_HUMAN | RNFWILRLPPGSKGEAPKVPVTFDDVAVYFSELEWGKLEDWQKELY |
| | | KHVMRGNYETLVSLDYAISKPDILTRIERGEEPC |
| 353 | ZN417_HUMAN | AAAAPRRPTQQGTVTFEDVAVNFSQEEWCLLSEAQRCLYRDVMLEN |
| | | LALISSLGCWCGSKDEEAPCKQRISVQRESQSRT |
| 354 | ZN182_HUMAN | SGEDSGSFYSWQKAKREQGLVTFEDVAVDFTQEEWQYLNPPQRTLY |
| | | RDVMLETYSNLVFVGQQVTKPNLILKLEVEECPA |
| 355 | ZN823_HUMAN | DSVAFEDVAVNFTQEEWALLGPSQKSLYRNVMQETIRNLDCIEMKW |
| | | EDQNIGDQCQNAKRNLRSHTCEIKDDSQCGETFG |
| 356 | ZN177_HUMAN | AAGWLTTWSQNSVTFQEVAVDFSQEEWALLDPAQKNLYKDVMLENF |
| | | RNLASVGYQLCRHSLISKVDQEQLKTDERGILQG |
| 357 | ZN197_HUMAN | ENPRNQLMALMLLTAQPQELVMFEEVSVCFTSEEWACLGPIQRALY |
| | | WDVMLENYGNVTSLEWETMTENEEVTSKPSSSQR |
| 358 | ZN717_HUMAN | LETYNSLVSLQELVSFEEVAVHFTWEEWQDLDDAQRTLYRDVMLET |
| | | YSSLVSLGHCITKPEMIFKLEQGAEPWIVEETPN |
| 359 | ZN669_HUMAN | RHFRRPEPCREPLASPIQDSVAFEDVAVNFTQEEWALLDSSQKNLY |
| | | REVMQETCRNLASVGSQWKDQNIEDHFEKPGKDI |
| 360 | ZN256_HUMAN | AAAELTAPAQGIVTFEDVAVYFSWKEWGLLDEAQKCLYHDVMLENL |
| | | TLTTSLGGSGAGDEEAPYQQSTSPQRVSQVRIPK |
| 361 | ZN251_HUMAN | AATFQLPGHQEMPLTFQDVAVYFSQAEGRQLGPQQRALYRDVMLEN |
| | | YGNVASLGFPVPKPELISQLEQGKELWVLNLLGA |
| 362 | CBX4_HUMAN | RSEAGEPPSSLQVKPETPASAAVAVAAAAAPTTTAEKPPAEAQDEP |
| | | AESLSEFKPFFGNIIITDVTANCLTVTFKEYVTV |
| 363 | PCGF2_HUMAN | HRTTRIKITELNPHLMCALCGGYFIDATTIVECLHSFCKTCIVRYL |
| | | ETNKYCPMCDVQVHKTRPLLSIRSDKTLQDIVYK |
| 364 | CDY2_HUMAN | ASQEFEVEAIVDKRQDKNGNTQYLVRWKGYDKQDDTWEPEQHLMNC |
| | | EKCVHDFNRRQTEKQKKLTWTTTSRIFSNNARRR |
| 365 | CDYL2_HUMAN | ASGDLYEVERIVDKRKNKKGKWEYLIRWKGYGSTEDTWEPEHHLLH |
| | | CEEFIDEFNGLHMSKDKRIKSGKQSSTSKLLRDS |
| 366 | HERC2_HUMAN | TLIRKADLENHNKDGGFWTVIDGKVYDIKDFQTQSLTGNSILAQFA |
| | | GEDPVVALEAALQFEDTRESMHAFCVGQYLEPDQ |
| 367 | ZN562_HUMAN | EKTKIGTMVEDHRSNSYQDSVTFDDVAVEFTPEEWALLDTTQKYLY |
| | | RDVMLENYMNLASVDFFFCLTSEWEIQPRTKRSS |
| 368 | ZN461_HUMAN | AHELVMERDVAIDVSQEEWECLNPAQRNLYKEVMLENYSNLVSLGL |
| | | SVSKPAVISSLEQGKEPWMVVREETGRWCPGTWK |
| 369 | Z324A_HUMAN | AFEDVAVYFSQEEWGLLDTAQRALYRRVMLDNFALVASLGLSTSRP |
| | | RVVIQLERGEEPWVPSGTDTTLSRTTYRRRNPGS |
| 370 | ZN766_HUMAN | AQLRRGHLTFRDVAIEFSQEEWKCLDPVQKALYRDVMLENYRNLVS |
| | | LGICLPDLSIISMMKQRTEPWTVENEMKVAKNPD |
| 371 | ID2_HUMAN | SDHSLGISRSKTPVDDPMSLLYNMNDCYSKLKELVPSIPQNKKVSK |
| | | MEILQHVIDYILDLQIALDSHPTIVSLHHQRPGQ |
| 372 | TOX_HUMAN | KDPNEPQKPVSAYALFFRDTQAAIKGQNPNATFGEVSKIVASMWDG |
| | | LGEEQKQVYKKKTEAAKKEYLKQLAAYRASLVSK |
| 373 | ZN274_HUMAN | QEEKQEDAAICPVTVLPEEPVTFQDVAVDFSREEWGLLGPTQRTEY |
| | | RDVMLETFGHLVSVGWETTLENKELAPNSDIPEE |
| 374 | SCMH1_HUMAN | DASRLSGRDPSSWTVEDVMQFVREADPQLGPHADLERKHEIDGKAL |
| | | LLLRSDMMMKYMGLKLGPALKLSYHIDRLKQGKF |
| 375 | ZN214_HUMAN | AVTFEDVTIIFTWEEWKFLDSSQKRLYREVMWENYTNVMSVENWNE |
| | | SYKSQEEKFRYLEYENFSYWQGWWNAGAQMYENQ |
| 376 | CBX7_HUMAN | ELSAIGEQVFAVESIRKKRVRKGKVEYLVKWKGWPPKYSTWEPEEH |
| | | ILDPRLVMAYEEKEERDRASGYRKRGPKPKRLLL |
| 377 | ID1_HUMAN | GGAGARLPALLDEQQVNVLLYDMNGCYSRLKELVPTLPQNRKVSKV |
| | | EILQHVIDYIRDLQLELNSESEVGTPGGRGLPVR |
| 378 | CREM_HUMAN | VVMAASPGSLHSPQQLAEEATRKRELRLMKNREAAKECRRRKKEYV |
| | | KCLESRVAVLEVQNKKLIEELETLKDICSPKTDY |
| 379 | SCX_HUMAN | GGGPGGRPGREPRQRHTANARERDRINSVNTAFTALRTLIPTEPAD |
| | | RKLSKIETLRLASSYISHLGNVLLAGEACGDGQP |
| 380 | ASCL1_HUMAN | SGFGYSLPQQQPAAVARRNERERNRVKLVNLGFATLREHVPNGAAN |
| | | KKMSKVETLRSAVEYIRALQQLLDEHDAVSAAFQ |
| 381 | ZN764_HUMAN | APLPPRDPNGAGPEWREPGAVSFADVAVYFCREEWGCLRPAQRALY |
| | | RDVMRETYGHLSALGIGGNKPALISWVEEEAELW |
| 382 | SCML2_HUMAN | KQGFSKDPSTWSVDEVIQFMKHTDPQISGPLADLFRQHEIDGKALF |
| | | LLKSDVMMKYMGLKLGPALKLCYYIEKLKEGKYS |
| 383 | TWST1_HUMAN | SGGGSPQSYEELQTQRVMANVRERQRTQSLNEAFAALRKIIPTLPS |
| | | DKLSKIQTLKLAARYIDFLYQVLQSDELDSKMAS |
| 384 | CREB1_HUMAN | IAPGVVMASSPALPTQPAEEAARKREVRLMKNREAARECRRKKKEY |
| | | VKCLENRVAVLENQNKTLIEELKALKDLYCHKSD |
| 385 | TERF1_HUMAN | SRIPVSKSQPVTPEKHRARKRQAWLWEEDKNLRSGVRKYGEGNWSK |
| | | ILLHYKFNNRTSVMLKDRWRTMKKLKLISSDSED |
| 386 | ID3_HUMAN | SLAIARGRGKGPAAEEPLSLLDDMNHCYSRLRELVPGVPRGTQLSQ |
| | | VEILQRVIDYILDLQVVLAEPAPGPPDGPHLPIQ |
| 387 | CBX8_HUMAN | GSGPPSSGGGLYRDMGAQGGRPSLIARIPVARILGDPEEESWSPSL |
| | | TNLEKVVVTDVTSNFLTVTIKESNTDQGFFKEKR |
| 388 | CBX4_HUMAN | ELPAVGEHVFAVESIEKKRIRKGRVEYLVKWRGWSPKYNTWEPEEN |
| | | ILDPRLLIAFQNRERQEQLMGYRKRGPKPKPLVV |
| 389 | GSX1_HUMAN | VDSSSNQLPSSKRMRTAFTSTQLLELEREFASNMYLSRLRRIEIAT |
| | | YLNLSEKQVKIWFQNRRVKHKKEGKGSNHRGGGG |
| 390 | NKX22_HUMAN | TPGGGGDAGKKRKRRVLFSKAQTYELERRFRQQRYLSAPEREHLAS |
| | | LIRLTPTQVKIWFQNHRYKMKRARAEKGMEVTPL |
| 391 | ATF1_HUMAN | QTVVMTSPVTLTSQTTKTDDPQLKREIRLMKNREAARECRRKKKEY |
| | | VKCLENRVAVLENQNKTLIEELKTLKDLYSNKSV |
| 392 | TWST2_HUMAN | KGSPSAQSFEELQSQRILANVRERQRTQSLNEAFAALRKIIPTLPS |
| | | DKLSKIQTLKLAARYIDFLYQVLQSDEMDNKMTS |
| 393 | ZNF17_HUMAN | NLTEDYMVFEDVAIHFSQEEWGILNDVQRHLHSDVMLENFALLSSV |
| | | GCWHGAKDEEAPSKQCVSVGVSQVTTLKPALSTQ |
| 394 | TOX3_HUMAN | KDPNEPQKPVSAYALFFRDTQAAIKGQNPNATFGEVSKIVASMWDS |
| | | LGEEQKQVYKRKTEAAKKEYLKALAAYRASLVSK |
| 395 | TOX4_HUMAN | KDPNEPQKPVSAYALFFRDTQAAIKGQNPNATFGEVSKIVASMWDS |
| | | LGEEQKQVYKRKTEAAKKEYLKALAAYKDNQECQ |
| 396 | ZMYM3_HUMAN | LDGSTWDFCSEDCKSKYLLWYCKAARCHACKRQGKLLETIHWRGQI |
| | | RHFCNQQCLLRFYSQQNQPNLDTQSGPESLLNSQ |
| 397 | I2BP1_HUMAN | ASVQASRRQWCYLCDLPKMPWAMVWDFSEAVCRGCVNFEGADRIEL |
| | | LIDAARQLKRSHVLPEGRSPGPPALKHPATKDLA |
| 398 | RHXF1_HUMAN | MEGPQPENMQPRTRRTKFTLLQVEELESVFRHTQYPDVPTRRELAE |
| | | NLGVTEDKVRVWFKNKRARCRRHQRELMLANELR |
| 399 | SSX2_HUMAN | PKIMPKKPAEEGNDSEEVPEASGPQNDGKELCPPGKPTTSEKIHER |
| | | SGPKRGEHAWTHRLRERKQLVIYEEISDPEEDDE |
| 400 | I2BPL_HUMAN | SAAQVSSSRRQSCYLCDLPRMPWAMIWDFSEPVCRGCVNYEGADRI |
| | | EFVIETARQLKRAHGCFQDGRSPGPPPPVGVKTV |
| 401 | ZN680_HUMAN | PGPPGSLEMGPLTFRDVAIEFSLEEWQCLDTAQRNLYRKVMFENYR |
| | | NLVFLGIAVSKPHLITCLEQGKEPWNRKRQEMVA |
| 402 | CBX1_HUMAN | NKKKVEEVLEEEEEEYVVEKVLDRRVVKGKVEYLLKWKGFSDEDNT |
| | | WEPEENLDCPDLIAEFLQSQKTAHETDKSEGGKR |
| 403 | TRI68_HUMAN | LANVVEKVRLLRLHPGMGLKGDLCERHGEKLKMFCKEDVLIMCEAC |
| | | SQSPEHEAHSVVPMEDVAWEYKWELHEALEHLKK |
| 404 | HXA13_HUMAN | VVSHPSDASSYRRGRKKRVPYTKVQLKELEREYAINKFITKDKRRR |
| | | ISATTNLSERQVTIWFQNRRVKEKKVINKLKTTS |
| 405 | PHC3_HUMAN | ENSDLLPVAQTEPSIWTVDDVWAFIHSLPGCQDIADEFRAQEIDGQ |
| | | ALLLLKEDHLMSAMNIKLGPALKICARINSLKES |
| 406 | TCF24_HUMAN | AGPGGGSRSGSGRPAAANAARERSRVQTLRHAFLELQRTLPSVPPD |
| | | TKLSKLDVLLLATTYIAHLTRSLQDDAEAPADAG |
| 407 | CBX3_HUMAN | QNGKSKKVEEAEPEEFVVEKVLDRRVVNGKVEYFLKWKGFTDADNT |
| | | WEPEENLDCPELIEAFLNSQKAGKEKDGTKRKSL |
| 408 | HXB13_HUMAN | QHPPDACAFRRGRKKRIPYSKGQLRELEREYAANKFITKDKRRKIS |
| | | AATSLSERQITIWFQNRRVKEKKVLAKVKNSATP |
| 409 | HEY1_HUMAN | SMSPTTSSQILARKRRRGIIEKRRRDRINNSLSELRRLVPSAFEKQ |
| | | GSAKLEKAEILQMTVDHLKMLHTAGGKGYFDAHA |
| 410 | PHC2_HUMAN | LVGMGHHFLPSEPTKWNVEDVYEFIRSLPGCQEIAEEFRAQEIDGQ |
| | | ALLLLKEDHLMSAMNIKLGPALKIYARISMLKDS |
| 411 | ZNF81_HUMAN | PANEDAPQPGEHGSACEVSVSFEDVTVDFSREEWQQLDSTQRRLYQ |
| | | DVMLENYSHLLSVGFEVPKPEVIFKLEQGEGPWT |
| 412 | FIGLA_HUMAN | GYSSTENLQLVLERRRVANAKERERIKNLNRGFARLKALVPFLPQS |
| | | RKPSKVDILKGATEYIQVLSDLLEGAKDSKKQDP |
| 413 | SAM11_HUMAN | EEAPAPEDVTKWTVDDVCSFVGGLSGCGEYTRVFREQGIDGETLPL |
| | | LTEEHLLTNMGLKLGPALKIRAQVARRLGRVFYV |
| 414 | KMT2B_HUMAN | GGTLAHTPRRSLPSHHGKKMRMARCGHCRGCLRVQDCGSCVNCLDK |
| | | PKFGGPNTKKQCCVYRKCDKIEARKMERLAKKGR |
| 415 | HEY2_HUMAN | LNSPTTTSQIMARKKRRGIIEKRRRDRINNSLSELRRLVPTAFEKQ |
| | | GSAKLEKAEILQMTVDHLKMLQATGGKGYFDAHA |
| 416 | JDP2_HUMAN | QPVKSELDEEEERRKRRREKNKVAAARCRNKKKERTEFLQRESERL |
| | | ELMNAELKTQIEELKQERQQLILMLNRHRPTCIV |
| 417 | HXC13_HUMAN | LQPEVSSYRRGRKKRVPYTKVQLKELEKEYAASKFITKEKRRRISA |
| | | TTNLSERQVTIWFQNRRVKEKKVVSKSKAPHLHS |
| 418 | ASCL4_HUMAN | LPVPLDSAFEPAFLRKRNERERQRVRCVNEGYARLRDHLPRELADK |
| | | RLSKVETLRAAIDYIKHLQELLERQAWGLEGAAG |
| 419 | HHEX_HUMAN | SPFLQRPLHKRKGGQVRFSNDQTIELEKKFETQKYLSPPERKRLAK |
| | | MLQLSERQVKTWFQNRRAKWRRLKQENPQSNKKE |
| 420 | HERC2_HUMAN | IAIATGSLHCVCCTEDGEVYTWGDNDEGQLGDGTTNAIQRPRLVAA |
| | | LQGKKVNRVACGSAHTLAWSTSKPASAGKLPAQV |
| 421 | GSX2_HUMAN | GGSDASQVPNGKRMRTAFTSTQLLELEREFSSNMYLSRLRRIEIAT |
| | | YLNLSEKQVKIWFQNRRVKHKKEGKGTQRNSHAG |
| 422 | BIN1_HUMAN | RLDLPPGFMFKVQAQHDYTATDTDELQLKAGDVVLVIPFQNPEEQD |
| | | EGWLMGVKESDWNQHKELEKCRGVFPENFTERVP |
| 423 | ETV7_HUMAN | GICKLPGRLRIQPALWSREDVLHWLRWAEQEYSLPCTAEHGFEMNG |
| | | RALCILTKDDFRHRAPSSGDVLYELLQYIKTQRR |
| 424 | ASCL3_HUMAN | PNYRGCEYSYGPAFTRKRNERERQRVKCVNEGYAQLRHHLPEEYLE |
| | | KRLSKVETLRAAIKYINYLQSLLYPDKAETKNNP |
| 425 | PHC1_HUMAN | LHGINPVFLSSNPSRWSVEEVYEFIASLQGCQEIAEEFRSQEIDGQ |
| | | ALLLLKEEHLMSAMNIKLGPALKICAKINVLKET |
| 426 | OTP_HUMAN | QAGQQQGQQKQKRHRTRFTPAQLNELERSFAKTHYPDIFMREELAL |
| | | RIGLTESRVQVWFQNRRAKWKKRKKTTNVFRAPG |
| 427 | I2BP2_HUMAN | AAAVAVAAASRRQSCYLCDLPRMPWAMIWDFTEPVCRGCVNYEGAD |
| | | RVEFVIETARQLKRAHGCFPEGRSPPGAAASAAA |
| 428 | VGLL2_HUMAN | FSSQTPASIKEEEGSPEKERPPEAEYINSRCVLFTYFQGDISSVVD |
| | | EHFSRALSQPSSYSPSCTSSKAPRSSGPWRDCSF |
| 429 | HXA11_HUMAN | DKAGGSSGQRTRKKRCPYTKYQIRELEREFFFSVYINKEKRLQLSR |
| | | MLNLTDRQVKIWFQNRRMKEKKINRDRLQYYSAN |
| 430 | PDLI4_HUMAN | GAPLSGLQGLPECTRCGHGIVGTIVKARDKLYHPECFMCSDCGLNL |
| | | KQRGYFFLDERLYCESHAKARVKPPEGYDVVAVY |
| 431 | ASCL2_HUMAN | RRPATAETGGGAAAVARRNERERNRVKLVNLGFQALRQHVPHGGAS |
| | | KKLSKVETLRSAVEYIRALQRLLAEHDAVRNALA |
| 432 | CDX4_HUMAN | TVQVTGKTRTKEKYRVVYTDHQRLELEKEFHCNRYITIQRKSELAV |
| | | NLGLSERQVKIWFQNRRAKERKMIKKKISQFENS |
| 433 | ZN860_HUMAN | EEAAQKRKEKEPGMALPQGHLTERDVAIEFSLEEWKCLDPTQRALY |
| | | RAMMLENYRNLHSVDISSKCMMKKFSSTAQGNTE |
| 434 | LMBL4_HUMAN | DIRASQVARWTVDEVAEFVQSLLGCEEHAKCFKKEQIDGKAFLLLT |
| | | QTDIVKVMKIKLGPALKIYNSILMFRHSQELPEE |
| 435 | PDIP3_HUMAN | LSPLEGTKMTVNNLHPRVTEEDIVELFCVCGALKRARLVHPGVAEV |
| | | VFVKKDDAITAYKKYNNRCLDGQPMKCNLHMNGN |
| 436 | NKX25_HUMAN | DNAERPRARRRRKPRVLFSQAQVYELERRFKQQRYLSAPERDQLAS |
| | | VLKLTSTQVKIWFQNRRYKCKRQRQDQTLELVGL |
| 437 | CEBPB_HUMAN | SQVKSKAKKTVDKHSDEYKIRRERNNIAVRKSRDKAKMRNLETQHK |
| | | VLELTAENERLQKKVEQLSRELSTLRNLFKQLPE |
| 438 | ISL1_HUMAN | KRDYIRLYGIKCAKCSIGFSKNDFVMRARSKVYHIECFRCVACSRQ |
| | | LIPGDEFALREDGLFCRADHDVVERASLGAGDPL |
| 439 | CDX2_HUMAN | SLGSQVKTRTKDKYRVVYTDHQRLELEKEFHYSRYITIRRKAELAA |
| | | TLGLSERQVKIWFQNRRAKERKINKKKLQQQQQQ |
| 440 | PROP1_HUMAN | QGGQRGRPHSRRRHRTTFSPVQLEQLESAFGRNQYPDIWARESLAR |
| | | DTGLSEARIQVWFQNRRAKQRKQERSLLQPLAHL |
| 441 | SIN3B_HUMAN | DALTYLDQVKIRFGSDPATYNGFLEIMKEFKSQSIDTPGVIRRVSQ |
| | | LFHEHPDLIVGFNAFLPLGYRIDIPKNGKLNIQS |
| 442 | SMBT1_HUMAN | RLHLDSNPLKWSVADVVRFIRSTDCAPLARIFLDQEIDGQALLLLT |
| | | LPTVQECMDLKLGPAIKLCHHIERIKFAFYEQFA |
| 443 | HXC11_HUMAN | AKGAAPNAPRTRKKRCPYSKFQIRELEREFFFNVYINKEKRLQLSR |
| | | MLNLTDRQVKIWFQNRRMKEKKLSRDRLQYFSGN |
| 444 | HXC10_HUMAN | TTGNWLTAKSGRKKRCPYTKHQTLELEKEFLENMYLTRERRLEISK |
| | | TINLTDRQVKIWFQNRRMKLKKMNRENRIRELTS |
| 445 | PRS6A_HUMAN | YLVSNVIELLDVDPNDQEEDGANIDLDSQRKGKCAVIKTSTRQTYF |
| | | LPVIGLVDAEKLKPGDLVGVNKDSYLILETLPTE |
| 446 | VSX1_HUMAN | KASPTLGKRKKRRHRTVFTAHQLEELEKAFSEAHYPDVYAREMLAV |
| | | KTELPEDRIQVWFQNRRAKWRKREKRWGGSSVMA |
| 447 | NKX23_HUMAN | EESERPKPRSRRKPRVLFSQAQVFELERRFKQQRYLSAPEREHLAS |
| | | SLKLTSTQVKIWFQNRRYKCKRQRQDKSLELGAH |
| 448 | MTG16_HUMAN | VVPGSRQEEVIDHKLTEREWAEEWKHLNNLLNCIMDMVEKTRRSLT |
| | | VLRRCQEADREELNHWARRYSDAEDTKKGPAPAA |
| 449 | HMX3_HUMAN | ESPEKKPACRKKKTRTVFSRSQVFQLESTFDMKRYLSSSERAGLAA |
| | | SLHLTETQVKIWFQNRRNKWKRQLAAELEAANLS |
| 450 | HMX1_HUMAN | RGGVGVGGGRKKKTRTVFSRSQVFQLESTEDLKRYLSSAERAGLAA |
| | | SLQLTETQVKIWFQNRRNKWKRQLAAELEAASLS |
| 451 | KIF22_HUMAN | ELLAHGRQKILDLLNEGSARDLRSLQRIGPKKAQLIVGWRELHGPF |
| | | SQVEDLERVEGITGKQMESFLKANILGLAAGQRC |
| 452 | CSTF2_HUMAN | ESPYGETISPEDAPESISKAVASLPPEQMFELMKQMKLCVQNSPQE |
| | | ARNMLLQNPQLAYALLQAQVVMRIVDPEIALKIL |
| 453 | CEBPE_HUMAN | AGPLHKGKKAVNKDSLEYRLRRERNNIAVRKSRDKAKRRILETQQK |
| | | VLEYMAENERLRSRVEQLTQELDTLRNLFRQIPE |
| 454 | DLX2_HUMAN | IRIVNGKPKKVRKPRTIYSSFQLAALQRRFQKTQYLALPERAELAA |
| | | SLGLTQTQVKIWFQNRRSKFKKMWKSGEIPSEQH |
| 455 | ZMYM3 HUMAN | TVYQFCSPSCWTKFQRTSPEGGIHLSCHYCHSLFSGKPEVLDWQDQ |
| | | VFQFCCRDCCEDFKRLRGVVSQCEHCRQEKLLHE |
| 456 | PPARG_HUMAN | TMVDTEMPFWPTNFGISSVDLSVMEDHSHSFDIKPFTTVDFSSIST |
| | | PHYEDIPFTRTDPVVADYKYDLKLQEYQSAIKVE |
| 457 | PRIC1_HUMAN | GRHHAELLKPRCSACDEIIFADECTEAEGRHWHMKHFCCLECETVL |
| | | GGQRYIMKDGRPFCCGCFESLYAEYCETCGEHIG |
| 458 | UNC4_HUMAN | DPDKESPGCKRRRTRINFTGWQLEELEKAFNESHYPDVFMREALAL |
| | | RLDLVESRVQVWFQNRRAKWRKKENTKKGPGRPA |
| 459 | BARX2_HUMAN | TEQPTPRQKKPRRSRTIFTELQLMGLEKKFQKQKYLSTPDRLDLAQ |
| | | SLGLTQLQVKTWYQNRRMKWKKMVLKGGQEAPTK |
| 460 | ALX3_HUMAN | SMELAKNKSKKRRNRTTFSTFQLEELEKVFQKTHYPDVYAREQLAL |
| | | RTDLTEARVQVWFQNRRAKWRKRERYGKIQEGRN |
| 461 | TCF15_HUMAN | GGGGGAGPVVVVRQRQAANARERDRTQSVNTAFTALRTLIPTEPVD |
| | | RKLSKIETVRLASSYIAHLANVLLLGDSADDGQP |
| 462 | TERA_HUMAN | IDDTVEGITGNLFEVYLKPYFLEAYRPIRKGDIFLVRGGMRAVEFK |
| | | VVETDPSPYCIVAPDTVIHCEGEPIKREDEEESL |
| 463 | VSX2_HUMAN | SALNQTKKRKKRRHRTIFTSYQLEELEKAFNEAHYPDVYAREMLAM |
| | | KTELPEDRIQVWFQNRRAKWRKREKCWGRSSVMA |
| 464 | HXD12_HUMAN | DGLPWGAAPGRARKKRKPYTKQQIAELENEFLVNEFINRQKRKELS |
| | | NRLNLSDQQVKIWFQNRRMKKKRVVLREQALALY |
| 465 | CDX1_HUMAN | GGGGSGKTRTKDKYRVVYTDHQRLELEKEFHYSRYITIRRKSELAA |
| | | NLGLTERQVKIWFQNRRAKERKVNKKKQQQQQPP |
| 466 | TCF23_HUMAN | TRAGGLALGRSEASPENAARERSRVRTLRQAFLALQAALPAVPPDT |
| | | KLSKLDVLVLAASYIAHLTRTLGHELPGPAWPPF |
| 467 | ALX1_HUMAN | KCDSNVSSSKKRRHRTTFTSLQLEELEKVFQKTHYPDVYVREQLAL |
| | | RTELTEARVQVWFQNRRAKWRKRERYGQIQQAKS |
| 468 | HXA10_HUMAN | NAANWLTAKSGRKKRCPYTKHQTLELEKEFLENMYLTRERRLEISR |
| | | SVHLTDRQVKIWFQNRRMKLKKMNRENRIRELTA |
| 469 | RX_HUMAN | LSEEEQPKKKHRRNRTTFTTYQLHELERAFEKSHYPDVYSREELAG |
| | | KVNLPEVRVQVWFQNRRAKWRRQEKLEVSSMKLQ |
| 470 | CXXC5_HUMAN | HMAGLAEYPMQGELASAISSGKKKRKRCGMCAPCRRRINCEQCSSC |
| | | RNRKTGHQICKFRKCEELKKKPSAALEKVMLPTG |
| 471 | SCML1_HUMAN | SITKHPSTWSVEAVVLFLKQTDPLALCPLVDLFRSHEIDGKALLLL |
| | | TSDVLLKHLGVKLGTAVKLCYYIDRLKQGKCFEN |
| 472 | NFIL3_HUMAN | ACRRKREFIPDEKKDAMYWEKRRKNNEAAKRSREKRRLNDLVLENK |
| | | LIALGEENATLKAELLSLKLKFGLISSTAYAQEI |
| 473 | DLX6_HUMAN | EIRFNGKGKKIRKPRTIYSSLQLQALNHRFQQTQYLALPERAELAA |
| | | SLGLTQTQVKIWFQNKRSKFKKLLKQGSNPHESD |
| 474 | MTG8_HUMAN | GLHGTRQEEMIDHRLTDREWAEEWKHLDHLLNCIMDMVEKTRRSLT |
| | | VLRRCQEADREELNYWIRRYSDAEDLKKGGGSSS |
| 475 | CBX8_HUMAN | ELSAVGERVFAAEALLKRRIRKGRMEYLVKWKGWSQKYSTWEPEEN |
| | | ILDARLLAAFEEREREMELYGPKKRGPKPKTFLL |
| 476 | CEBPD_HUMAN | AREKSAGKRGPDRGSPEYRQRRERNNIAVRKSRDKAKRRNQEMQQK |
| | | LVELSAENEKLHQRVEQLTRDLAGLRQFFKQLPS |
| 477 | SEC13_HUMAN | SGGCDNLIKLWKEEEDGQWKEEQKLEAHSDWVRDVAWAPSIGLPTS |
| | | TIASCSQDGRVFIWTCDDASSNTWSPKLLHKEND |
| 478 | FIP1_HUMAN | VKGVDLDAPGSINGVPLLEVDLDSFEDKPWRKPGADLSDYFNYGEN |
| | | EDTWKAYCEKQKRIRMGLEVIPVTSTINKITAED |
| 479 | ALX4_HUMAN | KADSESNKGKKRRNRTTFTSYQLEELEKVFQKTHYPDVYAREQLAM |
| | | RTDLTEARVQVWFQNRRAKWRKRERFGQMQQVRT |
| 480 | LHX3_HUMAN | TAKQREAEATAKRPRTTITAKQLETLKSAYNTSPKPARHVREQLSS |
| | | ETGLDMRVVQVWFQNRRAKEKRLKKDAGRQRWGQ |
| 481 | PRIC2_HUMAN | GRHHAECLKPRCAACDEIIFADECTEAEGRHWHMKHFCCFECETVL |
| | | GGQRYIMKEGRPYCCHCFESLYAEYCDTCAQHIG |
| 482 | MAGI3_HUMAN | IIGGDRPDEFLQVKNVLKDGPAAQDGKIAPGDVIVDINGNCVLGHT |
| | | HADVVQMFQLVPVNQYVNLTLCRGYPLPDDSEDP |
| 483 | NELL1_HUMAN | CCPECDTRVTSQCLDQNGHKLYRSGDNWTHSCQQCRCLEGEVDCWP |
| | | LTCPNLSCEYTAILEGECCPRCVSDPCLADNITY |
| 484 | PRRX1_HUMAN | LNSEEKKKRKQRRNRTTFNSSQLQALERVFERTHYPDAFVREDLAR |
| | | RVNLTEARVQVWFQNRRAKFRRNERAMLANKNAS |
| 485 | MTG8R_HUMAN | GLNGGYQDELVDHRLTEREWADEWKHLDHALNCIMEMVEKTRRSMA |
| | | VLRRCQESDREELNYWKRRYNENTELRKTGTELV |
| 486 | RAX2_HUMAN | GPGEEAPKKKHRRNRTTFTTYQLHQLERAFEASHYPDVYSREELAA |
| | | KVHLPEVRVQVWFQNRRAKWRRQERLESGSGAVA |
| 487 | DLX3_HUMAN | VRMVNGKPKKVRKPRTIYSSYQLAALQRRFQKAQYLALPERAELAA |
| | | QLGLTQTQVKIWFQNRRSKFKKLYKNGEVPLEHS |
| 488 | DLX1_HUMAN | EVRFNGKGKKIRKPRTIYSSLQLQALNRRFQQTQYLALPERAELAA |
| | | SLGLTQTQVKIWFQNKRSKFKKLMKQGGAALEGS |
| 489 | NKX26_HUMAN | GRSEQPKARQRRKPRVLFSQAQVLALERRFKQQRYLSAPEREHLAS |
| | | ALQLTSTQVKIWFQNRRYKCKRQRQDKSLELAGH |
| 490 | NAB1_HUMAN | LPRTLGELQLYRILQKANLLSYFDAFIQQGGDDVQQLCEAGEEEFL |
| | | EIMALVGMASKPLHVRRLQKALRDWVINPGLENQ |
| 491 | SAMD7_HUMAN | NLSLDEDIQKWTVDDVHSFIRSLPGCSDYAQVFKDHAIDGETLPLL |
| | | TEEHLRGTMGLKLGPALKIQSQVSQHVGSMFYKK |
| 492 | PITX3_HUMAN | SPEDGSLKKKQRRQRTHFTSQQLQELEATFQRNRYPDMSTREEIAV |
| | | WTNLTEARVRVWFKNRRAKWRKRERSQQAELCKG |
| 493 | WDR5_HUMAN | SNLLVSASDDKTLKIWDVSSGKCLKTLKGHSNYVFCCNFNPQSNLI |
| | | VSGSFDESVRIWDVKTGKCLKTLPAHSDPVSAVH |
| 494 | MEOX2 HUMAN | GNYKSEVNSKPRKERTAFTKEQIRELEAEFAHHNYLTRLRRYEIAV |
| | | NLDLTERQVKVWFQNRRMKWKRVKGGQQGAAARE |
| 495 | NAB2_HUMAN | LPRTLGELQLYRVLQRANLLSYYETFIQQGGDDVQQLCEAGEEEFL |
| | | EIMALVGMATKPLHVRRLQKALREWATNPGLFSQ |
| 496 | DHX8_HUMAN | PEEPTIGDIYNGKVTSIMQFGCFVQLEGLRKRWEGLVHISELRREG |
| | | RVANVADVVSKGQRVKVKVLSFTGTKTSLSMKDV |
| 497 | FOXA2_HUMAN | YAFNHPFSINNLMSSEQQHHHSHHHHQPHKMDLKAYEQVMHYPGYG |
| | | SPMPGSLAMGPVTNKTGLDASPLAADTSYYQGVY |
| 498 | CBX6_HUMAN | TAAAGPAPPTAPEPAGASSEPEAGDWRPEMSPCSNVVVTDVTSNLL |
| | | TVTIKEFCNPEDFEKVAAGVAGAAGGGGSIGASK |
| 499 | EMX2_HUMAN | FLLHNALARKPKRIRTAFSPSQLLRLEHAFEKNHYVVGAERKQLAH |
| | | SLSLTETQVKVWFQNRRTKFKRQKLEEEGSDSQQ |
| 500 | CPSF6_HUMAN | KRIALYIGNLTWWTTDEDLTEAVHSLGVNDILEIKFFENRANGQSK |
| | | GFALVGVGSEASSKKLMDLLPKRELHGQNPVVTP |
| 501 | HXC12_HUMAN | SGAPWYPINSRSRKKRKPYSKLQLAELEGEFLVNEFITRQRRRELS |
| | | DRLNLSDQQVKIWFQNRRMKKKRLLLREQALSFF |
| 502 | KDM4B_HUMAN | SDNLYPESITSRDCVQLGPPSEGELVELRWTDGNLYKAKFISSVTS |
| | | HIYQVEFEDGSQLTVKRGDIFTLEEELPKRVRSR |
| 503 | LMBL3_HUMAN | GIPASKVSKWSTDEVSEFIQSLPGCEEHGKVFKDEQIDGEAFLLMT |
| | | QTDIVKIMSIKLGPALKIFNSILMFKAAEKNSHN |
| 504 | PHX2A_HUMAN | EPSGLHEKRKQRRIRTTFTSAQLKELERVFAETHYPDIYTREELAL |
| | | KIDLTEARVQVWFQNRRAKFRKQERAASAKGAAG |
| 505 | EMX1_HUMAN | LLLHGPFARKPKRIRTAFSPSQLLRLERAFEKNHYVVGAERKQLAG |
| | | SLSLSETQVKVWFQNRRTKYKRQKLEEEGPESEQ |
| 506 | NC2B_HUMAN | SSGNDDDLTIPRAAINKMIKETLPNVRVANDARELVVNCCTEFIHL |
| | | ISSEANEICNKSEKKTISPEHVIQALESLGFGSY |
| 507 | DLX4_HUMAN | ERRPQAPAKKLRKPRTIYSSLQLQHLNQRFQHTQYLALPERAQLAA |
| | | QLGLTQTQVKIWFQNKRSKYKKLLKQNSGGQEGD |
| 508 | SRY_HUMAN | NVQDRVKRPMNAFIVWSRDQRRKMALENPRMRNSEISKQLGYQWKM |
| | | LTEAEKWPFFQEAQKLQAMHREKYPNYKYRPRRK |
| 509 | ZN777_HUMAN | EITRLAVWAAVQAVERKLEAQAMRLLTLEGRTGTNEKKIADCEKTA |
| | | VEFANHLESKWVVLGILLQEYGLLQRRLENMENL |
| 510 | NELL1_HUMAN | CEKDIDECSEGIIECHNHSRCVNLPGWYHCECRSGFHDDGTYSLSG |
| | | ESCIDIDECALRTHTCWNDSACINLAGGEDCLCP |
| 511 | ZN398_HUMAN | AAISLWTVVAAVQAIERKVEIHSRRLLHLEGRTGTAEKKLASCEKT |
| | | VTELGNQLEGKWAVLGTLLQEYGLLQRRLENLEN |
| 512 | GATA3_HUMAN | GQNRPLIKPKRRLSAARRAGTSCANCQTTTTTLWRRNANGDPVCNA |
| | | CGLYYKLHNINRPLTMKKEGIQTRNRKMSSKSKK |
| 513 | BSH_HUMAN | HAELPGKHCRRRKARTVFSDSQLSGLEKRFEIQRYLSTPERVELAT |
| | | ALSLSETQVKTWFQNRRMKHKKQLRKSQDEPKAP |
| 514 | SF3B4_HUMAN | QDATVYVGGLDEKVSEPLLWELFLQAGPVVNTHMPKDRVTGQHQGY |
| | | GFVEFLSEEDADYAIKIMNMIKLYGKPIRVNKAS |
| 515 | TEAD1_HUMAN | PIDNDAEGVWSPDIEQSFQEALAIYPPCGRRKIILSDEGKMYGRNE |
| | | LIARYIKLRTGKTRTRKQVSSHIQVLARRKSRDF |
| 516 | TEAD3_HUMAN | GLDNDAEGVWSPDIEQSFQEALAIYPPCGRRKIILSDEGKMYGRNE |
| | | LIARYIKLRTGKTRTRKQVSSHIQVLARKKVREY |
| 517 | RGAP1_HUMAN | DSVGTPQSNGGMRLHDFVSKTVIKPESCVPCGKRIKFGKLSLKCRD |
| | | CRVVSHPECRDRCPLPCIPTLIGTPVKIGEGMLA |
| 518 | PHF1_HUMAN | SAPHSMTASSSSVSSPSPGLPRRSAPPSPLCRSLSPGTGGGVRGGV |
| | | GYLSRGDPVRVLARRVRPDGSVQYLVEWGGGGIF |
| 519 | FOXA1_HUMAN | GDPHYSFNHPFSINNLMSSSEQQHKLDFKAYEQALQYSPYGSTLPA |
| | | SLPLGSASVTTRSPIEPSALEPAYYQGVYSRPVL |
| 520 | GATA2_HUMAN | GQNRPLIKPKRRLSAARRAGTCCANCQTTTTTLWRRNANGDPVCNA |
| | | CGLYYKLHNVNRPLTMKKEGIQTRNRKMSNKSKK |
| 521 | FOXO3_HUMAN | DSLSGSSLYSTSANLPVMGHEKFPSDLDLDMFNGSLECDMESIIRS |
| | | ELMDADGLDENFDSLISTQNVVGLNVGNFTGAKQ |
| 522 | ZN212_HUMAN | TEISLWTVVAAIQAVEKKMESQAARLQSLEGRTGTAEKKLADCEKM |
| | | AVEFGNQLEGKWAVLGTLLQEYGLLQRRLENVEN |
| 523 | IRX4_HUMAN | MDSGTRRKNATRETTSTLKAWLQEHRKNPYPTKGEKIMLAIITKMT |
| | | LTQVSTWFANARRRLKKENKMTWPPRNKCADEKR |
| 524 | ZBED6_HUMAN | NIEKQIYLPSTRAKTSIVWHFFHVDPQYTWRAICNLCEKSVSRGKP |
| | | GSHLGTSTLQRHLQARHSPHWTRANKFGVASGEE |
| 525 | LHX4_HUMAN | AKQNDDSEAGAKRPRITITAKQLETLKNAYKNSPKPARHVREQLSS |
| | | ETGLDMRVVQVWFQNRRAKEKRLKKDAGRHRWGQ |
| 526 | SIN3A_HUMAN | DALSYLDQVKLQFGSQPQVYNDFLDIMKEFKSQSIDTPGVISRVSQ |
| | | LFKGHPDLIMGENTFLPPGYKIEVQTNDMVNVTT |
| 527 | RBBP7_HUMAN | DDHTVCLWDINAGPKEGKIVDAKAIFTGHSAVVEDVAWHLLHESLF |
| | | GSVADDQKLMIWDTRSNTTSKPSHLVDAHTAEVN |
| 528 | NKX61_HUMAN | GSILLDKDGKRKHTRPTFSGQQIFALEKTFEQTKYLAGPERARLAY |
| | | SLGMTESQVKVWFQNRRTKWRKKHAAEMATAKKK |
| 529 | TRI68_HUMAN | DPTALVEAIVEEVACPICMTFLREPMSIDCGHSFCHSCLSGLWEIP |
| | | GESQNWGYTCPLCRAPVQPRNLRPNWQLANVVEK |
| 530 | R51A1_HUMAN | QSLPKKVSLSSDTTRKPLEIRSPSAESKKPKWVPPAASGGSRSSSS |
| | | PLVVVSVKSPNQSLRLGLSRLARVKPLHPNATST |
| 531 | MB3L1_HUMAN | AKSSQRKQRDCVNQCKSKPGLSTSIPLRMSSYTFKRPVTRITPHPG |
| | | NEVRYHQWEESLEKPQQVCWQRRLQGLQAYSSAG |
| 532 | DLX5_HUMAN | VRMVNGKPKKVRKPRTIYSSFQLAALQRRFQKTQYLALPERAELAA |
| | | SLGLTQTQVKIWFQNKRSKIKKIMKNGEMPPEHS |
| 533 | NOTC1_HUMAN | LQCNNHACGWDGGDCSLNFNDPWKNCTQSLQCWKYFSDGHCDSQCN |
| | | SAGCLFDGFDCQRAEGQCNPLYDQYCKDHFSDGH |
| 534 | TERF2_HUMAN | ETWVEEDELFQVQAAPDEDSTTNITKKQKWTVEESEWVKAGVQKYG |
| | | EGNWAAISKNYPFVNRTAVMIKDRWRTMKRLGMN |
| 535 | ZN282_HUMAN | AEISLWTVVAAIQAVERKVDAQASQLLNLEGRTGTAEKKLADCEKT |
| | | AVEFGNHMESKWAVLGTLLQEYGLLQRRLENLEN |
| 536 | RGS12_HUMAN | LEKRTLFRLDLVPINRSVGLKAKPTKPVTEVLRPVVARYGLDLSGL |
| | | LVRLSGEKEPLDLGAPISSLDGQRVVLEEKDPSR |
| 537 | ZN840_HUMAN | PNCLSSSMQLPHGGGRHQELVRFRDVAVVFSPEEWDHLTPEQRNLY |
| | | KDVMLDNCKYLASLGNWTYKAHVMSSLKQGKEPW |
| 538 | SPI2B_HUMAN | DDYKEGDLRIMPESSESPPTEREPGGVVDGLIGKHVEYTKEDGSKR |
| | | IGMVIHQVEAKPSVYFIKFDDDFHIYVYDLVKKS |
| 539 | PAX7_HUMAN | SEPDLPLKRKQRRSRITFTAEQLEELEKAFERTHYPDIYTREELAQ |
| | | RTKLTEARVQVWFSNRRARWRKQAGANQLAAFNH |
| 540 | NKX62_HUMAN | AGGVLDKDGKKKHSRPTFSGQQIFALEKTFEQTKYLAGPERARLAY |
| | | SLGMTESQVKVWFQNRRTKWRKRHAVEMASAKKK |
| 541 | ASXL2_HUMAN | DVMSFSVTVTTIPASQAMNPSSHGQTIPVQAFSEENSIEGTPSKCY |
| | | CRLKAMIMCKGCGAFCHDDCIGPSKLCVSCLVVR |
| 542 | FOXO1_HUMAN | GGYSSVSSCNGYGRMGLLHQEKLPSDLDGMFIERLDCDMESIIRND |
| | | LMDGDTLDFNFDNVLPNQSFPHSVKTTTHSWVSG |
| 543 | GATA3_HUMAN | GGSPTGFGCKSRPKARSSTGRECVNCGATSTPLWRRDGTGHYLCNA |
| | | CGLYHKMNGQNRPLIKPKRRLSAARRAGTSCANC |
| 544 | GATA1_HUMAN | GQNRPLIRPKKRLIVSKRAGTQCTNCQTTTTTLWRRNASGDPVCNA |
| | | CGLYYKLHQVNRPLTMRKDGIQTRNRKASGKGKK |
| 545 | ZMYM5 HUMAN | PVALLRKQNFQPTAQQQLTKPAKITCANCKKPLQKGQTAYQRKGSA |
| | | HLFCSTTCLSSFSHKRTQNTRSIICKKDASTKKA |
| 546 | ZN783_HUMAN | TEITLWTVVAAIQALEKKVDSCLTRLLTLEGRTGTAEKKLADCEKT |
| | | AVEFGNQLEGKWAVLGTLLQEYGLLQRRLENVEN |
| 547 | SPI2B_HUMAN | KKQRGRPSSQPRRNIVGCRISHGWKEGDEPITQWKGTVLDQVPINP |
| | | SLYLVKYDGIDCVYGLELHRDERVLSLKILSDRV |
| 548 | LRP1_HUMAN | WTCDLDDDCGDRSDESASCAYPTCFPLTQFTCNNGRCININWRCDN |
| | | DNDCGDNSDEAGCSHSCSSTQFKCNSGRCIPEHW |
| 549 | MIXL1_HUMAN | PKGAAAPSASQRRKRTSFSAEQLQLLELVFRRTRYPDIHLRERLAA |
| | | LTLLPESRIQVWFQNRRAKSRRQSGKSFQPLARP |
| 550 | SGT1_HUMAN | KIKYDWYQTESQVVITLMIKNVQKNDVNVEFSEKELSALVKLPSGE |
| | | DYNLKLELLHPIIPEQSTFKVLSTKIEIKLKKPE |
| 551 | LMCD1 HUMAN | DPSKEVEYVCELCKGAAPPDSPVVYSDRAGYNKQWHPTCFVCAKCS |
| | | EPLVDLIYFWKDGAPWCGRHYCESLRPRCSGCDE |
| 552 | CEBPA_HUMAN | GSGAGKAKKSVDKNSNEYRVRRERNNIAVRKSRDKAKQRNVETQQK |
| | | VLELTSDNDRLRKRVEQLSRELDTLRGIFRQLPE |
| 553 | GATA2_HUMAN | GPASSFTPKQRSKARSCSEGRECVNCGATATPLWRRDGTGHYLCNA |
| | | CGLYHKMNGQNRPLIKPKRRLSAARRAGTCCANC |
| 554 | SOX14_HUMAN | KPSDHIKRPMNAFMVWSRGQRRKMAQENPKMHNSEISKRLGAEWKL |
| | | LSEAEKRPYIDEAKRLRAQHMKEHPDYKYRPRRK |
| 555 | WTIP_HUMAN | LYSGFQQTADKCSVCGHLIMEMILQALGKSYHPGCFRCSVCNECLD |
| | | GVPFTVDVENNIYCVRDYHTVFAPKCASCARPIL |
| 556 | PRP19_HUMAN | HPSQDLVFSASPDATIRIWSVPNASCVQVVRAHESAVTGLSLHATG |
| | | DYLLSSSDDQYWAFSDIQTGRVLTKVTDETSGCS |
| 557 | CBX6_HUMAN | ELSAVGERVFAAESIIKRRIRKGRIEYLVKWKGWAIKYSTWEPEEN |
| | | ILDSRLIAAFEQKERERELYGPKKRGPKPKTFLL |
| 558 | NKX11_HUMAN | RTGSDSKSGKPRRARTAFTYEQLVALENKFKATRYLSVCERLNLAL |
| | | SLSLTETQVKIWFQNRRTKWKKQNPGADTSAPTG |
| 559 | RBBP4_HUMAN | VWDLSKIGEEQSPEDAEDGPPELLFIHGGHTAKISDFSWNPNEPWV |
| | | ICSVSEDNIMQVWQMAENIYNDEDPEGSVDPEGQ |
| 560 | DMRT2_HUMAN | ERCTPAGGGAEPRKLSRTPKCARCRNHGVVSCLKGHKRFCRWRDCQ |
| | | CANCLLVVERQRVMAAQVALRRQQATEDKKGLSG |
| 561 | SMCA2_HUMAN | SQPGALIPGDPQAMSQPNRGPSPFSPVQLHQLRAQILAYKMLARGQ |
| | | PLPETLQLAVQGKRTLPGLQQQQQQQQQQQQQQQ |
| 562 | ZNF10 | MDAKSLTAWSRTLVTFKDVFVDFTREEWKLLDTAQQIVYRNVMLEN |
| | | YKNLVSLGYQLTKPDVILRLEKGEEPWLVEREIHQETHPDSETAFE |
| | | IKSSVSSRSIFKDKQSCDIKMEGMARNDLWYLSLEEVWKCRDQLDK |
| | | YQENPERHLRQVAFTQKKVLTQERVSESGKYGGNCLLPAQLVLREY |
| | | FHKRDSHTKSLKHDLVLNGHQDSCASNSNECGQTFCQNIHLIQFAR |
| | | THTGDKSYKCPDNDNSLTHGSSLGISKGIHREKPYECKECGKFFSW |
| | | RSNLTRHQLIHTGEKPYECKECGKSFSRSSHLIGHQKTHTGEEPYE |
| | | CKECGKSFSWFSHLVTHQRTHTGDKLYTCNQCGKSFVHSSRLIRHQ |
| | | RTHTGEKPYECPECGKSFRQSTHLILHQRTHVRVRPYECNECGKSY |
| | | SQRSHLVVHHRIHTGLKPFECKDCGKCFSRSSHLYSHQRTHTGEKP |
| | | YECHDCGKSFSQSSALIVHQRIHTGEKPYECCQCGKAFIRKNDLIK |
| | | HQRIHVGEETYKCNQCGIIFSQNSPFIVHQIAHTGEQFLTCNQCGT |
| | | ALVNTSNLIGYQTNHIRENAY |
| 563 | EED_HUMAN | MSEREVSTAPAGTDMPAAKKQKLSSDENSNPDLSGDENDDAVSIES |
| | | GTNTERPDTPTNTPNAPGRKSWGKGKWKSKKCKYSFKCVNSLKEDH |
| | | NQPLFGVQFNWHSKEGDPLVFATVGSNRVTLYECHSQGEIRLLQSY |
| | | VDADADENFYTCAWTYDSNTSHPLLAVAGSRGIIRIINPITMQCIK |
| | | HYVGHGNAINELKFHPRDPNLLLSVSKDHALRLWNIQTDTLVAIFG |
| | | GVEGHRDEVLSADYDLLGEKIMSCGMDHSLKLWRINSKRMMNAIKE |
| | | SYDYNPNKTNRPFISQKIHFPDFSTRDIHRNYVDCVRWLGDLILSK |
| | | SCENAIVCWKPGKMEDDIDKIKPSESNVTILGRFDYSQCDIWYMRF |
| | | SMDFWQKMLALGNQVGKLYVWDLEVEDPHKAKCTTLTHHKCGAAIR |
| | | QTSFSRDSSILIAVCDDASIWRWDRLR |
| 564 | RCOR1_HUMAN | MPAMVEKGPEVSGKRRGRNNAAASASAAAASAAASAACASPAATAA |
| | | SGAAASSASAAAASAAAAPNNGQNKSLAAAAPNGNSSSNSWEEGSS |
| | | GSSSDEEHGGGGMRVGPQYQAVVPDFDPAKLARRSQERDNLGMLVW |
| | | SPNQNLSEAKLDEYIAIAKEKHGYNMEQALGMLFWHKHNIEKSLAD |
| | | LPNFTPFPDEWTVEDKVLFEQAFSFHGKTFHRIQQMLPDKSIASLV |
| | | KFYYSWKKTRTKTSVMDRHARKQKREREESEDELEEANGNNPIDIE |
| | | VDQNKESKKEVPPTETVPQVKKEKHSTQAKNRAKRKPPKGMFLSQE |
| | | DVEAVSANATAATTVLRQLDMELVSVKRQIQNIKQTNSALKEKLDG |
| | | GIEPYRLPEVIQKCNARWTTEEQLLAVQAIRKYGRDFQAISDVIGN |
| | | KSVVQVKNFFVNYRRRFNIDEVLQEWEAEHGKEETNGPSNQKPVKS |
| | | PDNSIKMPEEEDEAPVLDVRYASAS |
| 565 | CRISPR-off | TGRTLVTFKDVFVDFTREEWKLLDTAQQIVYRNVMLENYKNLVSLG |
| | KRAB | YQLTKPDVILRLEKGEEPLEINLWITKFVKD |
| | (KOX1/ZNF10) | |
| 566 | N-Terminal | MYPYDVPDYASPKKKRKVGGGASMDAKSLTAWSRTLVTFKDVFVDF |
| | KRAB | TREEWKLLDTAQQIVYRNVMLENYKNLVSLGYQLTKPDVILRLEKG |
| | (KOX1/ZNF10) | EEPWLVEREIHQETHPDSETAFEIKSSV |
| 567 | C-Terminal | ALSPQHSAVTQGSIIKNKEGMDAKSLTAWSRTLVTFKDVFVDFTRE |
| | KRAB | EWKLLDTAQQIVYRNVMLENYKNLVSLGYQLTKPDVILRLEKGEEP |
| | (KOX1/ZNF10) | WLVEREIHQETHPDSETAFEIKSSV |
| 568 | KOX1/ZNF10 (aa | RTLVTFKDVFVDFTREEWKLLDTAQQIVYRNVMLENYKNLVSLGYQ |
| | 11-72) | LTKPDVILRLEKGEEP |
| 569 | KOX1/ZNF10 (aa | RTLVTFKDVFVDFTREEWKLLDTAQQIVYRNVMLENYKNLVSLGYQ |
| | 11-108) | LTKPDVILRLEKGEEPWLVEREIHQETHPDSETAFEIKSSVSSRSI |
| | | FKDKQS |
| 570 | KOX1/ZNF10 | RTLVTFKDVAVDFTQEEWQQLDPAQKIVYRDVMLENYSNLVSVGYQ |
| | variant | LTKPDVILRLEQKGEEPWLVEEEIHQETHPDSETAFEIKSSVSSRS |
| | | IFKDKQS |
| 571 | KOX1 KRAB- | RTLVTFKDVFVDFTREEWKLLDTAQQIVYRNVMLENYKNLVSLGYQ |
| | ZIM3 chimera | LTKPDVILRLEKGEEPWLEEEEVLGSGRAEKNGDIGGQIWKPKDVK |
| | | ESL |
| 572 | ZIM3-KOX1 | MNNSQGRVTFEDVTVNFTQGEWQRLNPEQRNLYRDVMLENYSNLVS |
| | KRAB chimera | VGQGETTKPDVILRLEQGKEPWLVEREIHQETHPDSETAFEIKSSV |
| | | SSRSIFKDKQS |
| 573 | human DNMT1 | MPARTAPARVPTLAVPAISLPDDVRRRLKDLERDSLTEKECVKEKL |
| | | NLLHEFLQTEIKNQLCDLETKLRKEELSEEGYLAKVKSLLNKDLSL |
| | | ENGAHAYNREVNGRLENGNQARSEARRVGMADANSPPKPLSKPRTP |
| | | RRSKSDGEAKPEPSPSPRITRKSTRQTTITSHFAKGPAKRKPQEES |
| | | ERAKSDESIKEEDKDQDEKRRRVTSRERVARPLPAEEPERAKSGTR |
| | | TEKEEERDEKEEKRLRSQTKEPTPKQKLKEEPDREARAGVQADEDE |
| | | DGDEKDEKKHRSQPKDLAAKRRPEEKEPEKVNPQISDEKDEDEKEE |
| | | KRRKTTPKEPTEKKMARAKTVMNSKTHPPKCIQCGQYLDDPLKYGQ |
| | | HPPDAVDEPQMLTNEKLSIFDANESGFESYEALPQHKLTCFSVYCK |
| | | HGHLCPIDTGLIEKNIELFFSGSAKPIYDDDPSLEGGVNGKNLGPI |
| | | NEWWITGFDGGEKALIGFSTSFAEYILMDPSPEYAPIFGLMQEKIY |
| | | ISKIVVEFLQSNSDSTYEDLINKIETTVPPSGLNLNRFTEDSLLRH |
| | | AQFVVEQVESYDEAGDSDEQPIFLTPCMRDLIKLAGVTLGQRRAQA |
| | | RRQTIRHSTREKDRGPTKATTTKLVYQIFDTFFAEQIEKDDREDKE |
| | | NAFKRRRCGVCEVCQQPECGKCKACKDMVKFGGSGRSKQACQERRC |
| | | PNMAMKEADDDEEVDDNIPEMPSPKKMHQGKKKKQNKNRISWVGEA |
| | | VKTDGKKSYYKKVCIDAETLEVGDCVSVIPDDSSKPLYLARVTALW |
| | | EDSSNGQMFHAHWFCAGTDTVLGATSDPLELFLVDECEDMQLSYIH |
| | | SKVKVIYKAPSENWAMEGGMDPESLLEGDDGKTYFYQLWYDQDYAR |
| | | FESPPKTQPTEDNKFKFCVSCARLAEMRQKEIPRVLEQLEDLDSRV |
| | | LYYSATKNGILYRVGDGVYLPPEAFTFNIKLSSPVKRPRKEPVDED |
| | | LYPEHYRKYSDYIKGSNLDAPEPYRIGRIKEIFCPKKSNGRPNETD |
| | | IKIRVNKFYRPENTHKSTPASYHADINLLYWSDEEAVVDFKAVQGR |
| | | CTVEYGEDLPECVQVYSMGGPNRFYFLEAYNAKSKSFEDPPNHARS |
| | | PGNKGKGKGKGKGKPKSQACEPSEPEIEIKLPKLRTLDVFSGCGGL |
| | | SEGFHQAGISDTLWAIEMWDPAAQAFRLNNPGSTVFTEDCNILLKL |
| | | VMAGETTNSRGQRLPQKGDVEMLCGGPPCQGFSGMNRFNSRTYSKF |
| | | KNSLVVSFLSYCDYYRPRFFLLENVRNFVSFKRSMVLKLTLRCLVR |
| | | MGYQCTFGVLQAGQYGVAQTRRRAIILAAAPGEKLPLFPEPLHVFA |
| | | PRACQLSVVVDDKKFVSNITRLSSGPFRTITVRDTMSDLPEVRNGA |
| | | SALEISYNGEPQSWFQRQLRGAQYQPILRDHICKDMSALVAARMRH |
| | | IPLAPGSDWRDLPNIEVRLSDGTMARKLRYTHHDRKNGRSSSGALR |
| | | GVCSCVEAGKACDPAARQFNTLIPWCLPHTGNRHNHWAGLYGRLEW |
| | | DGFFSTTVTNPEPMGKQGRVLHPEQHRVVSVRECARSQGFPDTYRL |
| | | FGNILDKHRQVGNAVPPPLAKAIGLEIKLCMLAKARESASAKIKEE |
| | | EAAKD |
| 574 | human DNMT3A | MPAMPSSGPGDTSSSAAEREEDRKDGEEQEEPRGKEERQEPSTTAR |
| | | KVGRPGRKRKHPPVESGDTPKDPAVISKSPSMAQDSGASELLPNGD |
| | | LEKRSEPQPEEGSPAGGQKGGAPAEGEGAAETLPEASRAVENGCCT |
| | | PKEGRGAPAEAGKEQKETNIESMKMEGSRGRLRGGLGWESSLRQRP |
| | | MPRLTFQAGDPYYISKRKRDEWLARWKREAEKKAKVIAGMNAVEEN |
| | | QGPGESQKVEEASPPAVQQPTDPASPTVATTPEPVGSDAGDKNATK |
| | | AGDDEPEYEDGRGFGIGELVWGKLRGFSWWPGRIVSWWMTGRSRAA |
| | | EGTRWVMWFGDGKFSVVCVEKLMPLSSFCSAFHQATYNKQPMYRKA |
| | | IYEVLQVASSRAGKLFPVCHDSDESDTAKAVEVQNKPMIEWALGGF |
| | | QPSGPKGLEPPEEEKNPYKEVYTDMWVEPEAAAYAPPPPAKKPRKS |
| | | TAEKPKVKEIIDERTRERLVYEVRQKCRNIEDICISCGSLNVTLEH |
| | | PLFVGGMCQNCKNCFLECAYQYDDDGYQSYCTICCGGREVLMCGNN |
| | | NCCRCFCVECVDLLVGPGAAQAAIKEDPWNCYMCGHKGTYGLLRRR |
| | | EDWPSRLQMFFANNHDQEFDPPKVYPPVPAEKRKPIRVLSLFDGIA |
| | | TGLLVLKDLGIQVDRYIASEVCEDSITVGMVRHQGKIMYVGDVRSV |
| | | TQKHIQEWGPFDLVIGGSPCNDLSIVNPARKGLYEGTGRLFFEFYR |
| | | LLHDARPKEGDDRPFFWLFENVVAMGVSDKRDISRFLESNPVMIDA |
| | | KEVSAAHRARYFWGNLPGMNRPLASTVNDKLELQECLEHGRIAKFS |
| | | KVRTITTRSNSIKQGKDQHFPVFMNEKEDILWCTEMERVFGFPVHY |
| | | TDVSNMSRLARQRLLGRSWSVPVIRHLFAPLKEYFACV |
| 575 | human DNMT3A | NHDQEFDPPKVYPPVPAEKRKPIRVLSLFDGIATGLLVLKDLGIQV |
| | catalytic domain | DRYIASEVCEDSITVGMVRHQGKIMYVGDVRSVTQKHIQEWGPFDL |
| | | VIGGSPCNDLSIVNPARKGLYEGTGRLFFEFYRLLHDARPKEGDDR |
| | | PFFWLFENVVAMGVSDKRDISRFLESNPVMIDAKEVSAAHRARYFW |
| | | GNLPGMNRPLASTVNDKLELQECLEHGRIAKFSKVRTITTRSNSIK |
| | | QGKDQHFPVFMNEKEDILWCTEMERVFGFPVHYTDVSNMSRLARQR |
| | | LLGRSWSVPVIRHLFAPLKEYFACV |
| 576 | human DNMT3B | MKGDTRHLNGEEDAGGREDSILVNGACSDQSSDSPPILEAIRTPEI |
| | | RGRRSSSRLSKREVSSLLSYTQDLTGDGDGEDGDGSDTPVMPKLER |
| | | ETRTRSESPAVRTRNNNSVSSRERHRPSPRSTRGRQGRNHVDESPV |
| | | EFPATRSLRRRATASAGTPWPSPPSSYLTIDLIDDTEDTHGTPQSS |
| | | STPYARLAQDSQQGGMESPQVEADSGDGDSSEYQDGKEFGIGDLVW |
| | | GKIKGFSWWPAMVVSWKATSKRQAMSGMRWVQWFGDGKFSEVSADK |
| | | LVALGLFSQHFNLATENKLVSYRKAMYHALEKARVRAGKTFPSSPG |
| | | DSLEDQLKPMLEWAHGGFKPTGIEGLKPNNTQPVVNKSKVRRAGSR |
| | | KLESRKYENKTRRRTADDSATSDYCPAPKRLKINCYNNGKDRGDED |
| | | QSREQMASDVANNKSSLEDGCLSCGRKNPVSFHPLFEGGLCQTCRD |
| | | RFLELFYMYDDDGYQSYCTVCCEGRELLLCSNTSCCRCFCVECLEV |
| | | LVGTGTAAEAKLQEPWSCYMCLPQRCHGVLRRRKDWNVRLQAFFTS |
| | | DIGLEYEAPKLYPAIPAARRRPIRVLSLEDGIATGYLVLKELGIKV |
| | | GKYVASEVCEESIAVGTVKHEGNIKYVNDVRNITKKNIEEWGPFDL |
| | | VIGGSPCNDLSNVNPARKGLYEGTGRLFFEFYHLLNYSRPKEGDDR |
| | | PFFWMFENVVAMKVGDKRDISRFLECNPVMIDAIKVSAAHRARYFW |
| | | GNLPGMNRPVIASKNDKLELQDCLEYNRIAKLKKVQTITTKSNSIK |
| | | QGKNQLFPVVMNGKEDVLWCTELERIFGFPVHYTDVSNMGRGARQK |
| | | LLGRSWSVPVIRHLFAPLKDYFACE |
| 577 | mouse DNMT3C | MRGGSRHLSNEEDVSGCEDCIIISGTCSDQSSDPKTVPLTQVLEAV |
| | | CTVENRGCRISSQPSKRKASSLISYVQDLTGDGDEDRDGEVGGSSG |
| | | SGTPVMPQLFCETRIPSKTPAPLSWQANTSASTPWLSPASPYPIID |
| | | LTDEDVIPQSISTPSVDWSQDSHQEGMDTTQVDAESRDGGNIEYQV |
| | | SADKLLLSQSCILAAFYKLVPYRESTYRTLEKARVRAGKACPSSPG |
| | | ESLEDQLKPMLEWAHGGFKPTGIEGLKPNKKQPENKSRRRTINDPA |
| | | ASESSPPKRLKTNSYGGKDRGEDEESREQMASDVTNNKGNLEDHCL |
| | | SCGRKDPVSFHPLFEGGLCQSCRDRFLELFYMYDEDGYQSYCTVCC |
| | | EGRELLLCSNTSCCRCFCVECLEVLVGAGTAEDVKLQEPWSCYMCL |
| | | PQRCHGVLRRRKDWNMRLQDFFTTDPDLEEFEPPKLYPAIPAAKRR |
| | | PIRVLSLEDGIATGYLVLKELGIKVEKYIASEVCAESIAVGTVKHE |
| | | GQIKYVDDIRNITKEHIDEWGPFDLVIGGSPCNDLSCVNPVRKGLF |
| | | EGTGRLFFEFYRLLNYSCPEEEDDRPFFWMFENVVAMEVGDKRDIS |
| | | RFLECNPVMIDAIKVSAAHRARYFWGNLPGMNRPVMASKNDKLELQ |
| | | DCLEFSRTAKLKKVQTITTKSNSIRQGKNQLFPVVMNGKDDVLWCT |
| | | ELERIFGFPEHYTDVSNMGRGARQKLLGRSWSVPVIRHLFAPLKDH |
| | | FACE |
| 578 | human DNMT3L | MAAIPALDPEAEPSMDVILVGSSELSSSVSPGTGRDLIAYEVKANQ |
| | | RNIEDICICCGSLQVHTQHPLFEGGICAPCKDKFLDALFLYDDDGY |
| | | QSYCSICCSGETLLICGNPDCTRCYCFECVDSLVGPGTSGKVHAMS |
| | | NWVCYLCLPSSRSGLLQRRRKWRSQLKAFYDRESENPLEMFETVPV |
| | | WRRQPVRVLSLFEDIKKELTSLGFLESGSDPGQLKHVVDVTDTVRK |
| | | DVEEWGPFDLVYGATPPLGHTCDRPPSWYLFQFHRLLQYARPKPGS |
| | | PRPFFWMFVDNLVLNKEDLDVASRFLEMEPVTIPDVHGGSLQNAVR |
| | | VWSNIPAIRSSRHWALVSEEELSLLAQNKQSSKLAAKWPTKLVKNC |
| | | FLPLREYFKYFSTELTSSL |
| 579 | human DNMT3L | NPLEMFETVPVWRRQPVRVLSLFEDIKKELTSLGFLESGSDPGQLK |
| | catalytic domain | HVVDVTDTVRKDVEEWGPFDLVYGATPPLGHTCDRPPSWYLFQFHR |
| | | LLQYARPKPGSPRPFFWMFVDNLVLNKEDLDVASRFLEMEPVTIPD |
| | | VHGGSLQNAVRVWSNIPAIRSRHWALVSEEELSLLAQNKQSSKLAA |
| | | KWPTKLVKNCFLPLREYFKYFSTELTSSL |
| 580 | mouse DNMT3L | MGSRETPSSCSKTLETLDLETSDSSSPDADSPLEEQWLKSSPALKE |
| | | DSVDVVLEDCKEPLSPSSPPTGREMIRYEVKVNRRSIEDICLCCGT |
| | | LQVYTRHPLFEGGLCAPCKDKFLESLFLYDDDGHQSYCTICCSGGT |
| | | LFICESPDCTRCYCFECVDILVGPGTSERINAMACWVCFLCLPFSR |
| | | SGLLQRRKRWRHQLKAFHDQEGAGPMEIYKTVSAWKRQPVRVLSLF |
| | | RNIDKVLKSLGFLESGSGSGGGTLKYVEDVTNVVRRDVEKWGPFDL |
| | | VYGSTQPLGSSCDRCPGWYMFQFHRILQYALPRQESQRPFFWIFMD |
| | | NLLLTEDDQETTTRFLQTEAVTLQDVRGRDYQNAMRVWSNIPGLKS |
| | | KHAPLTPKEEEYLQAQVRSRSKLDAPKVDLLVKNCLLPLREYFKYF |
| | | SQNSLPL |
| 581 | mouse DNMT3L | GPMEIYKTVSAWKRQPVRVLSLFRNIDKVLKSLGFLESGSGSGGGT |
| | catalytic domain | LKYVEDVTNVVRRDVEKWGPFDLVYGSTQPLGSSCDRCPGWYMFQF |
| | | HRILQYALPRQESQRPFFWIFMDNLLLTEDDQETTTRFLQTEAVTL |
| | | QDVRGRDYQNAMRVWSNIPGLKSKHAPLTPKEEEYLQAQVRSRSKL |
| | | DAPKVDLLVKNCLLPLREYFKYFSQNSLPL |
| 582 | Ailuropoda | MALSPTGTLSVETLDRSDPDPLDEGPWQATCEILLEPDAEHSTDVI |
| | melanoleuca | LVGSSELSAPASPGPRRDLLAYEVKVNQRDIEDVCICCGSLRVHTQ |
| | DNMT3L | HPLFEGGMCAPCKDKFLDCLFLYDDDGYQSYCSICCAGETLLICEN |
| | | PDCTRPSLMMKLRLFRECACLIFPSEGMLLQTVWFWKMTVVWQPGL |
| | | RHLPQENPLETYKTVPVWKREPVRVLSLFGDIRRELMSLGFLESGS |
| | | APGRLKHLDDVTDVVRKDVEGWGPFDLVYGSTPPIGHACDHPPVWY |
| | | LLQFHRILQYARPRPGSQQPFFWMFVDNLVLSQDDQTAATRFLEAD |
| | | PVTIQDVCGRAVRNTVHVWSNIPAVRSRHSALALCEELSLLAQDRQ |
| | | RTKPPAQGPAQLVKNCFLPLREYFKYFSTELTSSL |
| 583 | Ailuropoda | NPLETYKTVPVWKREPVRVLSLFGDIRRELMSLGFLESGSAPGRLK |
| | melanoleuca | HLDDVTDVVRKDVEGWGPFDLVYGSTPPIGHACDHPPVWYLLQFHR |
| | DNMT3L | ILQYARPRPGSQQPFFWMFVDNLVLSQDDQTAATRFLEADPVTIQD |
| | catalytic domain | VCGRAVRNTVHVWSNIPAVRSRHSALALCEELSLLAQDRQRTKPPA |
| | | QGPAQLVKNCFLPLREYFKYFSTELTSSL |
| 584 | Carlito syrichta | MALSCRRTLPLESLHSSNSDLASQLDKEQWRPPCETHGIPVAAAPV |
| | DNMT3L | LDLEAECSLDVILVGSSELSTSSSPRLGRDHIAYEVKVNQRNIEDI |
| | | CLCCGSFLVHTQHPLFEGGMCAPCKDKFLDTLFLYDEDGYQSYCSI |
| | | CCSGETLLICENPDCTRCYCFECLDTLVSPGTSEKVHAMSNWVCFL |
| | | CLPFTRSGLLQRRRKWRGQLKAFYDRESESSLEMYKTVPVWKREPV |
| | | RVLSLFGDIKKELMSLGFVETGSDPGRLRHLDDTTNIVRRNVEEWG |
| | | PFHLLYGATPPLGHTCDRPPGWYLFQFHRLLQYARPQPGSPQPFFW |
| | | MFVDNVMLTREDRAIASRFLETEPVTIPDIHGRALQNAVCVWSNIP |
| | | AVRSKHSALVSEEELSLLAQDRQRAKLPTQGPTKLVKNCFLPLREY |
| | | FKYFSTELTSFL |
| 585 | Carlito syrichta | SSLEMYKTVPVWKREPVRVLSLFGDIKKELMSLGFVETGSDPGRLR |
| | DNMT3L | HLDDTTNIVRRNVEEWGPFHLLYGATPPLGHTCDRPPGWYLFQFHR |
| | catalytic domain | LLQYARPQPGSPQPFFWMFVDNVMLTREDRAIASRFLETEPVTIPD |
| | | IHGRALQNAVCVWSNIPAVRSKHSALVSEEELSLLAQDRQRAKLPT |
| | | QGPTKLVKNCFLPLREYFKYFSTELTSFL |
| 586 | Meriones | MGSQETPSTRAKTPGTWNLESTDSSSPESLGHLEEQWANSSPDLKD |
| | unguiculatus | EHSKDVEPEDSKELISSASPPSGREIIRYEISVNQRNIEDICLCCG |
| | DNMT3L | TLQVYKQHPLFEGGICAPCKDKFLETFFLYDEDGHQSYCSICCSGG |
| | | TLFICESPDCTRCYCFECVDILVGPGTSERINAMPCWVCFLCLPFT |
| | | RSGLLQRRRKWRHQLKAFFDEGGASPLEMYKTVSAWKRKPMRVLSL |
| | | FKNIDKELKNLGFLESGSGSEEERLKYLEDVINVVRRDVEKWGPFD |
| | | LVYGSTRPRGSSCDHCPAWYMFQFHRILQYARPPSGSEQPFFWVFV |
| | | DNLLMTEDDQITADRFLQMKAVTLQDVRGRVLQNAVRVWSNIPGVK |
| | | SKHMALTEKEEQSLEAQAGTRTKLSAQKVDPLVKNCLLPLREYFKF |
| | | FSQNSLPLDK |
| 587 | Meriones | SPLEMYKTVSAWKRKPMRVLSLFKNIDKELKNLGFLESGSGSEEER |
| | unguiculatus | LKYLEDVINVVRRDVEKWGPFDLVYGSTRPRGSSCDHCPAWYMFQF |
| | DNMT3L | HRILQYARPPSGSEQPFFWVFVDNLLMTEDDQITADRFLQMKAVTL |
| | catalytic domain | QDVRGRVLQNAVRVWSNIPGVKSKHMALTEKEEQSLEAQAGTRTKL |
| | | SAQKVDPLVKNCLLPLREYFKFFSQNSLPLDK |
| 588 | Ochotona | MALPSPETLDSLDRVPASHPDEQHWTVCDNSDPILEVEAEGSMDVI |
| | princeps | LVDDSPAPSGRDRIELEVKVNQRSIEDLCLCCGSSQVHRQHPLFQG |
| | DNMT3L | GLCAPCKDKFLEALFLYDEDGYQSYCSICGLGDTLLVCESPDCTRG |
| | | YCFACVDGLVGAGSSGHMHTVSPWVCFLCVPGSRHGLLQRRRRWRT |
| | | QLKVFHEQEAAQPLEIYETVPACRRKPLRVLSLFEHIEKELASLGF |
| | | LETGSSPGRIRHLDDVTDVVRRDVEQWGPFDLVYGSTPPLGHASPR |
| | | SPGWYLFQFHRMLQYTQPTASTQRPFFWMFVDNLLLTRDDLVTATR |
| | | FLEVEPATLQDVRGRVLQGAMRVWSNIPAVNSRHTELAPEAETALL |
| | | AQSCRRAKASGEGLARLLKSCFLPLREYFKYFPQSPLPLRK |
| 589 | Ochotona | QPLEIYETVPACRRKPLRVLSLFEHIEKELASLGFLETGSSPGRIR |
| | princeps | HLDDVTDVVRRDVEQWGPFDLVYGSTPPLGHASPRSPGWYLFQFHR |
| | DNMT3L | MLQYTQPTASTQRPFFWMFVDNLLLTRDDLVTATRFLEVEPATLQD |
| | catalytic domain | VRGRVLQGAMRVWSNIPAVNSRHTELAPEAETALLAQSCRRAKASG |
| | | EGLARLLKSCFLPLREYFKYFPQSPLPLRK |
| 590 | Neosciurus | MGGPRPAAVEESPHEIYKTVPAWKREPMRVLSLFGDIGKELTSLGF |
| | carolinensis | LETGSEAGRLKHLEDVTDTVRRDVEEWGPFDLVYGSTPALGHSCDR |
| | DNMT3L | SPGWYLFQFHRLLQYARPRLGSPKPFFWMFVDNLLLTKDDQAIASR |
| | | FLEMEPVTLQDVHGRVLQNAVRVWTNVPAVKSRHSALASEEELLLV |
| | | QDGQRGRLPAQGPAALVKHCFLPLREYFKYFSQNTLPLYK |
| 591 | Neosciurus | SPHEIYKTVPAWKREPMRVLSLFGDIGKELTSLGFLETGSEAGRLK |
| | carolinensis | HLEDVTDTVRRDVEEWGPFDLVYGSTPALGHSCDRSPGWYLFQFHR |
| | DNMT3L | LLQYARPRLGSPKPFFWMFVDNLLLTKDDQAIASRFLEMEPVTLQD |
| | catalytic domain | VHGRVLQNAVRVWTNVPAVKSRHSALASEEELLLVQDGQRGRLPAQ |
| | | GPAALVKHCFLPLREYFKYFSQNTLPLYK |
| 592 | Bison bison | MARSSPGTLNLEIMDGSDPDPALPPDREQWPPPCEILLDPEPEHSL |
| | DNMT3L | DIILVGSSELSSPPSPGPRRDFIAYEVKVNQRDIEDVCICCGSLQL |
| | | HTQHPLFEGGMCAPCKDKFLECLFLYDDDGYQSYCSICCAGETLLI |
| | | CENPDCTRCYCFECVDTLVGPGTSGKVHAMSNWVCFLCLPFPRSGL |
| | | LQRRRKWRTWLKAFYDREAESPLVMYKTVPVWKREPIRVLSLFGDI |
| | | KKELTSLGFLEDGSKPGRLKHLDDVTNIVRRDIDEWGPFDLTYGST |
| | | PTLGHTCDHPPGWYVYQFHRILQYARPLPGSPQPFFWMFVDNLVLT |
| | | EEDLDVATRFLETDPVTIQDVRGRTVQNAVHVWSNIPAVKSRHSAL |
| | | VSQEELSLLAQDRQRVKSPVQGPATLVKNCFLPLREYFKYFSTELT |
| | | SSL |
| 593 | Bison bison | SPLVMYKTVPVWKREPIRVLSLFGDIKKELTSLGFLEDGSKPGRLK |
| | DNMT3L | HLDDVTNIVRRDIDEWGPFDLTYGSTPTLGHTCDHPPGWYVYQFHR |
| | catalytic domain | ILQYARPLPGSPQPFFWMFVDNLVLTEEDLDVATRFLETDPVTIQD |
| | | VRGRTVQNAVHVWSNIPAVKSRHSALVSQEELSLLAQDRQRVKSPV |
| | | QGPATLVKNCFLPLREYFKYFSTELTSSL |
| 594 | Equus | MALSSPGTLSLETLDSWDPDVAGQLDEERWQPSSEIVGRPMAAAPV |
| | przewalskii | LDLEEEPSMDIILVDSSELSSPPSPGPSRDMCICCGSFQVHTQHPL |
| | DNMT3L | FEGGMCAACKDKFLSCLFLYDDDGNQSYCSICCSGETLLICENPDC |
| | | TRCYCFECVDTLVSPRTSEKVQAMSNWVCFLCLPFPRSGLLQRRRK |
| | | WRGWLKAFYDQEAVRSRSAWGRRMRSGPHLVGFLWLLVAKCPSALE |
| | | SPLEMYKTVPVWKREPVRVLSLFGDIKKELTTLGFLENGSDPGRLK |
| | | HLDDVTNTVRRDVEEWGPFDLVYGSTPPLGHACDHPPGWYLFQFHR |
| | | VLQYARPRPGSPQAFFWMFVDNLVLTEDDRAVATRFLETDPVTIQD |
| | | VCGRAVRNAVHVWSNIPAVKSRHSALFSQEESFLRAQDRQRAKPPA |
| | | RGPAKLVKNCFLPLREYFKYFSTEFTSSL |
| 595 | Equus | SPLEMYKTVPVWKREPVRVLSLFGDIKKELTTLGFLENGSDPGRLK |
| | przewalskii | HLDDVTNTVRRDVEEWGPFDLVYGSTPPLGHACDHPPGWYLFQFHR |
| | DNMT3L | VLQYARPRPGSPQAFFWMFVDNLVLTEDDRAVATRFLETDPVTIQD |
| | catalytic domain | VCGRAVRNAVHVWSNIPAVKSRHSALFSQEESFLRAQDRQRAKPPA |
| | | RGPAKLVKNCFLPLREYFKYFSTEFTSSL |
| 596 | Mus caroli | MGSRETPSSFSKTLETLDLETSDSSSPDADSPLEEQWLKSSPALKE |
| | DNMT3L | DNVDMVLEDCKEPLSPSSPPTGREMIRYEVKVNRRSIEDICLCCGT |
| | | LQVYTQHPLFEGGICAPCKDKFLESLFLYDDDGHQSYCTICCSGGT |
| | | LFICESPDCTRCYCFECVDILVGPGTSERINAMACWVCFLCLPFSR |
| | | SGLLQRRKRWRHQLKAFHDQEGAGPMEIYKTVSTWKRQPVRVLSLF |
| | | GNIDKVLKSLGFLESGSGSGGGTLKYVEDVINVVRRDVEKWGPFDL |
| | | VYGSTQPLGSSCDRCPGWYMFQFHRILQYALPRQESQRPFFWIFMD |
| | | NLLMTEDDQETTARFLQTEAVTLQDVRGRDYQNVMRVWSNIPGLKS |
| | | KHVPLTPKEEEYLQAQVRTRSKLDAQKVDLLVKNCLLPLREYFKYF |
| | | S |
| 597 | Mus caroli | GPMEIYKTVSTWKRQPVRVLSLFGNIDKVLKSLGFLESGSGSGGGT |
| | DNMT3L | LKYVEDVTNVVRRDVEKWGPFDLVYGSTQPLGSSCDRCPGWYMFQF |
| | catalytic domain | HRILQYALPRQESQRPFFWIFMDNLLMTEDDQETTARFLQTEAVTL |
| | | QDVRGRDYQNVMRVWSNIPGLKSKHVPLTPKEEEYLQAQVRTRSKL |
| | | DAQKVDLLVKNCLLPLREYFKYFS |
| 598 | Pan troglodytes | MAAIPALDPEAEPSMDVILVGSSELSSSISPRTGRDLIAYEVKANQ |
| | DNMT3L | RNIEDICICCGSLQVHTQHPLFEGGICAPCKDKSLDALFLYDDDGY |
| | | QSYCSICCSGETLLICGNPDCTRCYCFECVDSLVGPGTSGKVHAMS |
| | | NWVCYLCLPSSRSGLLQRRRKWRSQLKAFYDRESENPLEMFETVPV |
| | | WRRQPVRVLSLFEDIKKELTSLGFLESGSDPGQLKHVVDVTDTVRK |
| | | DVEEWGPFDLVYGATPPLGHTCDRPPSWYLFQFHRLLQYARPKPGS |
| | | PRPFFWMFVDNLVLNKEDLDVASRFLEMEPVTIPDVHGGSLQNAVR |
| | | VWSNIPAIRSSRHWALVSEEELSLLAQNKQSSKLAAKWPTKLVKNC |
| | | FLPLREYFKYFSTELTSSL |
| 599 | Pan troglodytes | NPLEMFETVPVWRRQPVRVLSLFEDIKKELTSLGFLESGSDPGQLK |
| | DNMT3L | HVVDVTDTVRKDVEEWGPFDLVYGATPPLGHTCDRPPSWYLFQFHR |
| | catalytic domain | LLQYARPKPGSPRPFFWMFVDNLVLNKEDLDVASRFLEMEPVTIPD |
| | | VHGGSLQNAVRVWSNIPAIRSSRHWALVSEEELSLLAQNKQSSKLA |
| | | AKWPTKLVKNCFLPLREYFKYFSTELTSSL |
| 600 | human TRDMT1 | MEPLRVLELYSGVGGMHHALRESCIPAQVVAAIDVNTVANEVYKYN |
| | (DNMT2) | FPHTQLLAKTIEGITLEEFDRLSFDMILMSPPCQPFTRIGRQGDMT |
| | | DSRINSFLHILDILPRLQKLPKYILLENVKGFEVSSTRDLLIQTIE |
| | | NCGFQYQEFLLSPTSLGIPNSRLRYFLIAKLQSEPLPFQAPGQVLM |
| | | EFPKIESVHPQKYAMDVENKIQEKNVEPNISFDGSIQCSGKDAILF |
| | | KLETAEEIHRKNQQDSDLSVKMLKDFLEDDTDVNQYLLPPKSLLRY |
| | | ALLLDIVQPTCRRSVCFTKGYGSYIEGTGSVLQTAEDVQVENIYKS |
| | | LTNLSQEEQITKLLILKLRYFTPKEIANLLGFPPEFGFPEKITVKQ |
| | | RYRLLGNSLNVHVVAKLIKILYE |
| 601 | M. bacterium | MAEWYIPAIVSYQAIHNGFTLNKINHKIELQTMIDYLESKILSMNS |
| | methyltransferase | KEPVKRGFWYKKHLDEIRIVYTAVKMSEQEGNIFDVRTLFERGLSD |
| | | IDLLTYSFPCQDLSQQGKQKGMGRDSQTRSGLLWEIEKALDTSKKE |
| | | DLPKYLLMENVVALTHKVNAEELDEWMMKLESLGYKNDLRILNAGD |
| | | FGSSQARRRTFMISTLNEKVELPVGNKKPKSMNKILNDEPTRKDFL |
| | | PALDKFDLTEYKWTKSNINKAKLINYSTFNSEAYVYDSNFTGPTLT |
| | | ASGANSRIKFEYNGKIRKIGAEEAYAYMGFKKSDYIKVNKLNYLNE |
| | | TKMIYTCGNSISVEVLRSIMTNINNNFKENK |
| 602 | M. marinum | MLFLIGTFKYVLIYITKVIRIFEAFAGIGAQRKALRNIKSNYEVSG |
| | methyltransferase | MAEWYIPAIVSYQAIHNGFTLSRVDKKTKLTEMIKYLESKTLSMDS |
| | | KEPVRTGYWFKKHKDMVRIVYSAVKLSEAEGNIFDVRTLHERKLED |
| | | IDLLTYSFPCQDLSQQGKQRGMKKDSGTRSGLLWEIEKALEATPKD |
| | | KLPKYLLMENVVALTHKTNKKDLDNWKRKLRSLGYYNDINVLNAGD |
| | | FGSSQARRRAFMISTLDSKVTLPLGDKKPQAISKILNKETRSQDEM |
| | | PALDEYEKTDFKRILSNIKKCKLIDYTSFNSEAYVYDPKYTGPTLT |
| | | ASGANSRIKFTHQGKMRKINAEEAYRYMGFSTNDYKKVNNLNFLSE |
| | | TKMIYTCGNSISVEVLEEIMLKIIREDNNG |
| 603 | S. chinense | MKKIRLFEAFAGIGSQRRALKSVVGNNFEIAGLAEWYVPAIVMYQI |
| | methyltransferase | INNDFSKKNVLDNVPRDEVIDYLNSKCLSWDSKKPVSKNEWNRKSQ |
| | | DILNVIYSAVKKSEEEGNIFDVRTLHERTLESIDILTYSFPCQDLS |
| | | QQGIQKGMKKNSGTRSGLLWEIEKAIDNTPKNNLPKILLMENVPAL |
| | | LNKTNELELKEWLIKLENMGYKNSIGILNAADFGSPQARRRVFMIS |
| | | SRNKKIELPVGKSKPGKLNDILEKNVEDKFIMTNLEKYDFSEFSLT |
| | | KSNIKKCSLINYTKFNSEAYVYDPDFTGPTLTASGANSRIKIYDKG |
| | | FIRRMSPLESFRYMGEDDEDYKKIDEFEFLTDTQKIFVCGNSISIE |
| | | VLKAIFERIDSNE |
| 604 | M. penetrans M | MNSNKDKIKVIKVFEAFAGIGSQFKALKNIARSKNWEIQHSGMVEW |
| | MpeI | FVDAIVSYVAIHSKNFNPKIEQLDKDILSISNDSKMPISEYGIKKI |
| | | NNTIKASYLNYAKKHENNLFDIKKVNKDNFPKNIDIFTYSFPCQDL |
| | | SVQGLQKGIDKELNTRSGLLWEIERILEEIKNSFSKEEMPKYLLME |
| | | NVKNLLSHKNKKNYNTWLKQLEKFGYKSKTYLLNSKNEDNCQNRER |
| | | VFCLSIRDDYLEKTGFKFKELEKVKNPPKKIKDILVDSSNYKYLNL |
| | | NKYETTTFRETKSNIISRSLKNYTTFNSENYVYNINGIGPTLTASG |
| | | ANSRIKIETQQGVRYLTPLECFKYMQFDVNDFKKVQSTNLISENKM |
| | | IYIAGNSIPVKILEAIFNTLEFVNNEE |
| 605 | S. monobiae M | MSKVENKTKKLRVFEAFAGIGAQRKALEKVRKDEYEIVGLAEWYVP |
| | SssI | AIVMYQAIHNNFHTKLEYKSVSREEMIDYLENKTLSWNSKNPVSNG |
| | | YWKRKKDDELKIIYNAIKLSEKEGNIFDIRDLYKRILKNIDLLTYS |
| | | FPCQDLSQQGIQKGMKRGSGTRSGLLWEIERALDSTEKNDLPKYLL |
| | | MENVGALLHKKNEEELNQWKQKLESLGYQNSIEVLNAADFGSSQAR |
| | | RRVFMISTLNEFVELPKGDKKPKSIKKVLNKIVSEKDILNNLLKYN |
| | | LTEFKKTKSNINKASLIGYSKFNSEGYVYDPEFTGPTLTASGANSR |
| | | IKIKDGSNIRKMNSDETFLYIGFDSQDGKRVNEIEFLTENQKIFVC |
| | | GNSISVEVLEAIIDKIGG |
| 606 | H. parainfluenzae | MKDVLDDNLLEEPAAQYSLFEPESNPNLREKFTFIDLFAGIGGFRI |
| | M HpaII | AMQNLGGKCIFSSEWDEQAQKTYEANFGDLPYGDITLEETKAFIPE |
| | | KFDILCAGFPCQAFSIAGKRGGFEDTRGTLFFDVAEIIRRHQPKAF |
| | | FLENVKGLKNHDKGRTLKTILNVLREDLGYFVPEPAIVNAKNFGVP |
| | | QNRERIYIVGFHKSTGVNSFSYPEPLDKIVTFADIREEKTVPTKYY |
| | | LSTQYIDTLRKHKERHESKGNGFGYEIIPDDGIANAIVVGGMGRER |
| | | NLVIDHRITDFTPTTNIKGEVNREGIRKMTPREWARLQGFPDSYVI |
| | | PVSDASAYKQFGNSVAVPAIQATGKKILEKLGNLYD |
| 607 | A. luteus M AluI | MSKANAKYSFVDLFAGIGGFHAALAATGGVCEYAVEIDREAAAVYE |
| | | RNWNKPALGDITDDANDEGVTLRGYDGPIDVLTGGFPCQPFSKSGA |
| | | QHGMAETRGTLFWNIARIIEEREPTVLILENVRNLVGPRHRHEWLT |
| | | IIETLRFFGYEVSGAPAIFSPHLLPAWMGGTPQVRERVFITATLVP |
| | | ERMRDERIPRTETGEIDAEAIGPKPVATMNDRFPIKKGGTELFHPG |
| | | DRKSGWNLLISGIIREGDPEPSNVDLRLTETETLWIDAWDDLESTI |
| | | RRATGRPLEGFPYWADSWTDFRELSRLVVIRGFQAPEREVVGDRKR |
| | | YVARTDMPEGFVPASVTRPAIDETLPAWKQSHLRRNYDFFERHFAE |
| | | VVAWAYRWGVYTDLFPASRRKLEWQAQDAPRLWDTVMHFRPSGIRA |
| | | KRPTYLPALVAITQTSIVGPLERRLSPRETARLQGLPEWFDFGEQR |
| | | AAATYKQMGNGVNVGVVRHILREHVRRDRALLKLTPAGQRIINAVL |
| | | ADEPDATVGALGAAE |
| 608 | H. aegyptius M | MNLISLFSGAGGLDLGFQKAGFRIICANEYDKSIWKTYESNHSAKL |
| | HaeIII | IKGDISKISSDEFPKCDGIIGGPPCQSWSEGGSLRGIDDPRGKLFY |
| | | EYIRILKQKKPIFFLAENVKGMMAQRHNKAVQEFIQEFDNAGYDVH |
| | | IILLNANDYGVAQDRKRVFYIGFRKELNINYLPPIPHLIKPTFKDV |
| | | IWDLKDNPIPALDKNKINGNKCIYPNHEYFIGSYSTIFMSRNRVRQ |
| | | WNEPAFTVQASGRQCQLHPQAPVMLKVSKNLNKFVEGKEHLYRRLT |
| | | VRECARVQGFPDDFIFHYESLNDGYKMIGNAVPVNLAYEIAKTIKS |
| | | ALEICKGN |
| 609 | H. haemolyticus M | MIEIKDKQLTGLRFIDLFAGLGGFRLALESCGAECVYSNEWDKYAQ |
| | HhaI | EVYEMNFGEKPEGDITQVNEKTIPDHDILCAGFPCQAFSISGKQKG |
| | | FEDSRGTLFFDIARIVREKKPKVVFMENVKNFASHDNGNTLEVVKN |
| | | TMNELDYSFHAKVLNALDYGIPQKRERIYMICFRNDLNIQNFQFPK |
| | | PFELNTFVKDLLLPDSEVEHLVIDRKDLVMTNQEIEQTTPKTVRLG |
| | | IVGKGGQGERIYSTRGIAITLSAYGGGIFAKTGGYLVNGKTRKLHP |
| | | RECARVMGYPDSYKVHPSTSQAYKQFGNSVVINVLQYIAYNIGSSL |
| | | NEKPY |
| 610 | Moraxella M MspI | MKPEILKLIRSKLDLTQKQASEIIEVSDKTWQQWESGKTEMHPAYY |
| | | SFLQEKLKDKINFEELSAQKTLQKKIFDKYNQNQITKNAEELAEIT |
| | | HIEERKDAYSSDFKFIDLFSGIGGIRQSFEVNGGKCVFSSEIDPFA |
| | | KFTYYTNFGVVPFGDITKVEATTIPQHDILCAGFPCQPFSHIGKRE |
| | | GFEHPTQGTMFHEIVRIIETKKTPVLFLENVPGLINHDDGNTLKVI |
| | | IETLEDMGYKVHHTVLDASHFGIPQKRKRFYLVAFLNQNIHFEFPK |
| | | PPMISKDIGEVLESDVTGYSISEHLQKSYLFKKDDGKPSLIDKNTT |
| | | GAVKTLVSTYHKIQRLTGTFVKDGETGIRLLTTNECKAIMGFPKDF |
| | | VIPVSRTQMYRQMGNSVVVPVVTKIAEQISLALKTVNQQSPQENFE |
| | | LELV |
| 611 | Ascobolus Masc1 | MSERRYEAGMTVALHEGSFLKIQRVYIRQYHADNRREHMLVGPLFR |
| | | RTKYLKALSKKVNEVAIVHESIHVPVQDVIGVRELIITNRPFPECR |
| | | KGDEHTGRLVCRWVYNLDERAKGREYKKQRYIRRITEAEADPEYRV |
| | | EDRVLRRRWFQEGYIGDEISYKEHGNGDIVDIRSESPLQVLDGWGG |
| | | DLVDLENGEETSIPGPCRSASSYGRLMKPPLAQAADSNTSRKYTFG |
| | | DTFCGGGGVSLGARQAGLEVKWAFDMNPNAGANYRRNFPNTDFFLA |
| | | EAEQFIQLSVGISQHVDILHLSPPCQTFSRAHTIAGKNDENNEASF |
| | | FAVVNLIKAVRPRLFTVEETDGIMDRQSRQFIDTALMGITELGYSF |
| | | RICVLNAIEYGVCQNRKRLIIIGAAPGEELPPFPLPTHQDFFSKDP |
| | | RRDLLPAVTLDDALSTITPESTDHHLNHVWQPAEWKTPYDAHRPFK |
| | | NAIRAGGGEYDIYPDGRRKFTVRELACIQGFPDEYEFVGTLTDKRR |
| | | IIGNAVPPPLSAAIMSTLRQWMTEKDFERME |
| 612 | Arabidopsis | MVENGAKAAKRKKRPLPEIQEVEDVPRTRRPRRAAACTSFKEKSIR |
| | MET1 | VCEKSATIEVKKQQIVEEEFLALRLTALETDVEDRPTRRLNDFVLF |
| | | DSDGVPQPLEMLEIHDIFVSGAILPSDVCTDKEKEKGVRCTSFGRV |
| | | EHWSISGYEDGSPVIWISTELADYDCRKPAASYRKVYDYFYEKARA |
| | | SVAVYKKLSKSSGGDPDIGLEELLAAVVRSMSSGSKYFSSGAAIID |
| | | FVISQGDFIYNQLAGLDETAKKHESSYVEIPVLVALREKSSKIDKP |
| | | LQRERNPSNGVRIKEVSQVAESEALTSDQLVDGTDDDRRYAILLQD |
| | | EENRKSMQQPRKNSSSGSASNMFYIKINEDEIANDYPLPSYYKTSE |
| | | EETDELILYDASYEVQSEHLPHRMLHNWALYNSDLRFISLELLPMK |
| | | QCDDIDVNIFGSGVVTDDNGSWISLNDPDSGSQSHDPDGMCIFLSQ |
| | | IKEWMIEFGSDDIISISIRTDVAWYRLGKPSKLYAPWWKPVLKTAR |
| | | VGISILTFLRVESRVARLSFADVTKRLSGLQANDKAYISSDPLAVE |
| | | RYLVVHGQIILQLFAVYPDDNVKRCPFVVGLASKLEDRHHTKWIIK |
| | | KKKISLKELNLNPRAGMAPVASKRKAMQATTTRLVNRIWGEFYSNY |
| | | SPEDPLQATAAENGEDEVEEEGGNGEEEVEEEGENGLTEDTVPEPV |
| | | EVQKPHTPKKIRGSSGKREIKWDGESLGKTSAGEPLYQQALVGGEM |
| | | VAVGGAVTLEVDDPDEMPAIYFVEYMFESTDHCKMLHGRFLQRGSM |
| | | TVLGNAANERELFLINECMTTQLKDIKGVASFEIRSRPWGHQYRKK |
| | | NITADKLDWARALERKVKDLPTEYYCKSLYSPERGGFFSLPLSDIG |
| | | RSSGFCTSCKIREDEEKRSTIKLNVSKTGFFINGIEYSVEDFVYVN |
| | | PDSIGGLKEGSKTSFKSGRNIGLRAYVVCQLLEIVPKESRKADLGS |
| | | FDVKVRRFYRPEDVSAEKAYASDIQELYFSQDTVVLPPGALEGKCE |
| | | VRKKSDMPLSREYPISDHIFFCDLFFDTSKGSLKQLPANMKPKFST |
| | | IKDDTLLRKKKGKGVESEIESEIVKPVEPPKEIRLATLDIFAGCGG |
| | | LSHGLKKAGVSDAKWAIEYEEPAGQAFKQNHPESTVFVDNCNVILR |
| | | AIMEKGGDQDDCVSTTEANELAAKLTEEQKSTLPLPGQVDFINGGP |
| | | PCQGFSGMNRFNQSSWSKVQCEMILAFLSFADYFRPRYFLLENVRT |
| | | FVSFNKGQTFQLTLASLLEMGYQVRFGILEAGAYGVSQSRKRAFIW |
| | | AAAPEEVLPEWPEPMHVFGVPKLKISLSQGLHYAAVRSTALGAPFR |
| | | PITVRDTIGDLPSVENGDSRINKEYKEVAVSWFQKEIRGNTIALTD |
| | | HICKAMNELNLIRCKLIPTRPGADWHDLPKRKVTLSDGRVEEMIPF |
| | | CLPNTAERHNGWKGLYGRLDWQGNFPTSVTDPQPMGKVGMCFHPEQ |
| | | HRILTVRECARSQGFPDSYEFAGNINHKHRQIGNAVPPPLAFALGR |
| | | KLKEALHLKKSPQHQP |
| 613 | Ascobolus Masc2 | MELTPELSGVSTDLGGGGSIFAHWRMKEESPAPTEILDDLNVLEWE |
| | | KTTRDYSKEDLRIADQLFSIEDEHQSLPFETADAEDGTPTEEEEEK |
| | | ELPMRTLDNFVLYDASDLELAALDLIGTELNIHAVGTVGPIYTEGE |
| | | EDEQEDEDEDVSPPVRTGTQATSASVTQMTVELYIRNIVQYEFCEN |
| | | DDGTVETWIQTTNAHYKLLQPAKCYTSLYRPVNDCLNVITAIITLA |
| | | PESTTMSLKDLLKVMDDKAQAVSYEEVERMSEFIVQHLDQWMETAP |
| | | KKKSKLIEKSKVYIDLNNLAGIDMVSGVRPPPVRRVTGRSSAPKKR |
| | | IVRNMNDAVLLHQNETTVTNWIHQLSAGMFGRALNVLGAETADVEN |
| | | LTCDPASAKFVVPQRRLHKRLKWETRGHIPVSEEEYKHIYQGKKYA |
| | | KFFEAVRAVDESKLTIKLGDLVYVLDQDPKVTQTQFATAGREGRKK |
| | | GAEKEKIQVRFGRVLSIRQPDSNSKDAQNVFIHVQWLVLGCDTILQ |
| | | EMASRRELFLTDSCDTVFADVIYGVAKLTPLGAKDIPTVEFHESMA |
| | | TMMGENEFFVRFKYNYQDGSFTDLKDVDAEQIGTLQPRVNTHRNPG |
| | | YCSNCRIKYDNERTGDKWIYENDTEGEPRLFRSSKGWCIYAQEFVY |
| | | LQPVEKQPGTTFRVGYISEINKSSVIVELLARVDDDDKSGHISYSD |
| | | PRHLYFTGTDIKVTFDKIIRKCFVFHDSGDQKAKAPLMYGTLQRDL |
| | | YYYRYEKRKGKAELVPVREIRSIHEQTLNDWESRTQIERHGAVSGK |
| | | KLKGLDIFAGCGGLTLGLDLSGAVDTKWDIEFAPSAANTLALNFPD |
| | | AQVFNQCANVLLSRAIQSEDEGSLDIEYDLQGRVLPDLPKKGEVDE |
| | | IYGGPPCQGFSGVNRYKKGNDIKNSLVATFLSYVDHYKPRFVLLEN |
| | | VKGLITTKLGNSKNAEGKWEGGISNGVVKFIYRTLISMNYQCRIGL |
| | | VQSGEYGVPQSRPRVIFLAARMGERLPDLPEPMHAFEVLDSQYALP |
| | | HIKRYHTTQNGVAPLPRITIGEAVSDLPKFQYANPGVWPRHDPYSS |
| | | AKAQPSDKTIEKFSVSKATSFVGYLLQPYHSRPQSEFQRRLRTKLV |
| | | PSDEPAEKTSLLTTKLVTAHVTRLENKETTQRIVCVPMWPGADHRS |
| | | LPKEMRPWCLVDPNSQAEKHRFWPGLFGRLGMEDFFSTALTDVQPC |
| | | GKQGKVLHPTQRRVYTVRELARAQGFPDWFAFTDGDADSGLGGVKK |
| | | WHRNIGNAVPVPLGEQIGRCIGYSVWWKDDMIAQLREDGADEDEEM |
| | | IDGNDQWVEELNTQMAADMPGLPLLVTHLLNLCVYRRLYGPNAKEF |
| | | LPARVYDKKLEGGRRRLVWAML |
| 614 | Neurospora Dim2 | MDSPDRSHGGMFIDVPAETMGFQEDYLDMFASVLSQGLAKEGDYAH |
| | | HQPLPAGKEECLEPIAVATTITPSPDDPQLQLQLELEQQFQTESGL |
| | | NGVDPAPAPESEDEADLPDGFSDESPDDDFVVQRSKHITVDLPVST |
| | | LINPRSTFQRIDENDNLVPPPQSTPERVAVEDLLKAAKAAGKNKED |
| | | YIEFELHDFNFYVNYAYHPQEMRPIQLVATKVLHDKYYFDGVLKYG |
| | | NTKHYVTGMQVLELPVGNYGASLHSVKGQIWVRSKHNAKKEIYYLL |
| | | KKPAFEYQRYYQPFLWIADLGKHVVDYCTRMVERKREVTLGCFKSD |
| | | FIQWASKAHGKSKAFQNWRAQHPSDDFRTSVAANIGYIWKEINGVA |
| | | GAKRAAGDQLFRELMIVKPGQYFRQEVPPGPVVTEGDRTVAATIVT |
| | | PYIKECFGHMILGKVLRLAGEDAEKEKEVKLAKRLKIENKNATKAD |
| | | TKDDMKNDTATESLPTPLRSLPVQVLEATPIESDIVSIVSSDLPPS |
| | | ENNPPPLTNGSVKPKAKANPKPKPSTQPLHAAHVKYLSQELVNKIK |
| | | VGDVISTPRDDSSNTDTKWKPTDTDDHRWFGLVQRVHTAKTKSSGR |
| | | GLNSKSFDVIWFYRPEDTPCCAMKYKWRNELFLSNHCTCQEGHHAR |
| | | VKGNEVLAVHPVDWFGTPESNKGEFFVRQLYESEQRRWITLQKDHL |
| | | TCYHNQPPKPPTAPYKPGDTVLATLSPSDKFSDPYEVVEYFTQGEK |
| | | ETAFVRLRKLLRRRKVDRQDAPANELVYTEDLVDVRAERIVGKCIM |
| | | RCFRPDERVPSPYDRGGTGNMFFITHRQDHGRCVPLDTLPPTLRQG |
| | | FNPLGNLGKPKLRGMDLYCGGGNFGRGLEEGGVVEMRWANDIWDKA |
| | | IHTYMANTPDPNKTNPFLGSVDDLLRLALEGKFSDNVPRPGEVDFI |
| | | AAGSPCPGFSLLTQDKKVLNQVKNQSLVASFASFVDFYRPKYGVLE |
| | | NVSGIVQTFVNRKQDVLSQLFCALVGMGYQAQLILGDAWAHGAPQS |
| | | RERVFLYFAAPGLPLPDPPLPSHSHYRVKNRNIGFLCNGESYVQRS |
| | | FIPTAFKFVSAGEGTADLPKIGDGKPDACVRFPDHRLASGITPYIR |
| | | AQYACIPTHPYGMNFIKAWNNGNGVMSKSDRDLFPSEGKTRTSDAS |
| | | VGWKRLNPKTLFPTVTTTSNPSDARMGPGLHWDEDRPYTVQEMRRA |
| | | QGYLDEEVLVGRTTDQWKLVGNSVSRHMALAIGLKFREAWLGTLYD |
| | | ESAVVATATATATTAAAVGVTVPVMEEPGIGTTESSRPSRSPVHTA |
| | | VDLDDSKSERSRSTTPATVLSTSSAAGDGSANAAGLEDDDNDDMEM |
| | | MEVTRKRSSPAVDEEGMRPSKVQKVEVTVASPASRRSSRQASRNPT |
| | | ASPSSKASKATTHEAPAPEELESDAESYSETYDKEGEDGDYHSGHE |
| | | DQYSEEDEEEEYAEPETMTVNGMTIVKL |
| 615 | Drosophila | MVFRVLELFSGIGGMHYAFNYAQLDGQIVAALDVNTVANAVYAHNY |
| | dDnmt2 | GSNLVKTRNIQSLSVKEVTKLQANMLLMSPPCQPHTRQGLQRDTED |
| | | KRSDALTHLCGLIPECQELEYILMENVKGFESSQARNQFIESLERS |
| | | GFHWREFILTPTQFNVPNTRYRYYCIARKGADFPFAGGKIWEEMPG |
| | | AIAQNQGLSQIAEIVEENVSPDFLVPDDVLTKRVLVMDIIHPAQSR |
| | | SMCFTKGYTHYTEGTGSAYTPLSEDESHRIFELVKEIDTSNQDASK |
| | | SEKILQQRLDLLHQVRLRYFTPREVARLMSFPENFEFPPETTNRQK |
| | | YRLLGNSINVKVVGELIKLLTIK |
| 616 | S. pombe Pmt1 | MLSTKRLRVLELYSGIGGMHYALNLANIPADIVCAIDINPQANEIY |
| | | NLNHGKLAKHMDISTLTAKDFDAFDCKLWTMSPSCQPFTRIGNRKD |
| | | ILDPRSQAFLNILNVLPHVNNLPEYILIENVQGFEESKAAEECRKV |
| | | LRNCGYNLIEGILSPNQFNIPNSRSRWYGLARLNFKGEWSIDDVFQ |
| | | FSEVAQKEGEVKRIRDYLEIERDWSSYMVLESVLNKWGHQFDIVKP |
| | | DSSSCCCFTRGYTHLVQGAGSILQMSDHENTHEQFERNRMALQLRY |
| | | FTAREVARLMGFPESLEWSKSNVTEKCMYRLLGNSINVKVVSYLIS |
| | | LLLEPLNF |
| 617 | Arabidopsis | MVMSHIFLISQIQEVEHGDSDDVNWNTDDDELAIDNFQFSPSPVHI |
| | DRM1 | SATSPNSIQNRISDETVASFVEMGFSTQMIARAIEETAGANMEPMM |
| | | ILETLFNYSASTEASSSKSKVINHFIAMGFPEEHVIKAMQEHGDED |
| | | VGEITNALLTYAEVDKLRESEDMNININDDDDDNLYSLSSDDEEDE |
| | | LNNSSNEDRILQALIKMGYLREDAAIAIERCGEDASMEEVVDFICA |
| | | AQMARQFDEIYAEPDKKELMNNNKKRRTYTETPRKPNTDQLISLPK |
| | | EMIGFGVPNHPGLMMHRPVPIPDIARGPPFFYYENVAMTPKGVWAK |
| | | ISSHLYDIVPEFVDSKHFCAAARKRGYIHNLPIQNRFQIQPPQHNT |
| | | IQEAFPLTKRWWPSWDGRTKLNCLLTCIASSRLTEKIREALERYDG |
| | | ETPLDVQKWVMYECKKWNLVWVGKNKLAPLDADEMEKLLGFPRDHT |
| | | RGGGISTTDRYKSLGNSFQVDTVAYHLSVLKPLFPNGINVLSLFTG |
| | | IGGGEVALHRLQIKMNVVVSVEISDANRNILRSFWEQTNQKGILRE |
| | | FKDVQKLDDNTIERLMDEYGGFDLVIGGSPCNNLAGGNRHHRVGLG |
| | | GEHSSLFFDYCRILEAVRRKARHMRR |
| 618 | Arabadopsis | MVIWNNDDDDFLEIDNFQSSPRSSPIHAMQCRVENLAGVAVTTSSL |
| | DRM2 | SSPTETTDLVQMGFSDEVFATLFDMGFPVEMISRAIKETGPNVETS |
| | | VIIDTISKYSSDCEAGSSKSKAIDHFLAMGFDEEKVVKAIQEHGED |
| | | NMEAIANALLSCPEAKKLPAAVEEEDGIDWSSSDDDTNYTDMLNSD |
| | | DEKDPNSNENGSKIRSLVKMGFSELEASLAVERCGENVDIAELTDE |
| | | LCAAQMAREFSEFYTEHEEQKPRHNIKKRRFESKGEPRSSVDDEPI |
| | | RLPNPMIGFGVPNEPGLITHRSLPELARGPPFFYYENVALTPKGVW |
| | | ETISRHLFEIPPEFVDSKYFCVAARKRGYIHNLPINNRFQIQPPPK |
| | | YTIHDAFPLSKRWWPEWDKRTKLNCILTCTGSAQLTNRIRVALEPY |
| | | NEEPEPPKHVQRYVIDQCKKWNLVWVGKNKAAPLEPDEMESILGFP |
| | | KNHTRGGGMSRTERFKSLGNSFQVDTVAYHLSVLKPIFPHGINVLS |
| | | LFTGIGGGEVALHRLQIKMKLVVSVEISKVNRNILKDFWEQTNQTG |
| | | ELIEFSDIQHLINDTIEGLMEKYGGFDLVIGGSPCNNLAGGNRVSR |
| | | VGLEGDQSSLFFEYCRILEVVRARMRGS |
| 619 | Arabadopsis | MAARNKQKKRAEPESDLCFAGKPMSVVESTIRWPHRYQSKKTKLQA |
| | CMT1 | PTKKPANKGGKKEDEEIIKQAKCHFDKALVDGVLINLNDDVYVTGL |
| | | PGKLKFIAKVIELFEADDGVPYCRFRWYYRPEDTLIERFSHLVQPK |
| | | RVFLSNDENDNPLTCIWSKVNIAKVPLPKITSRIEQRVIPPCDYYY |
| | | DMKYEVPYLNFTSADDGSDASSSLSSDSALNCFENLHKDEKELLDL |
| | | YSGCGAMSTGFCMGASISGVKLITKWSVDINKFACDSLKINHPETE |
| | | VRNEAAEDFLALLKEWKRLCEKFSLVSSTEPVESISELEDEEVEEN |
| | | DDIDEASTGAELEPGEFEVEKFLGIMFGDPQGTGEKTLQLMVRWKG |
| | | YNSSYDTWEPYSGLGNCKEKLKEYVIDGFKSHLLPLPGTVYTVCGG |
| | | PPCQGISGYNRYRNNEAPLEDQKNQQLLVFLDIIDFLKPNYVLMEN |
| | | VVDLLRFSKGFLARHAVASFVAMNYQTRLGMMAAGSYGLPQLRNRV |
| | | FLWAAQPSEKLPPYPLPTHEVAKKENTPKEFKDLQVGRIQMEFLKL |
| | | DNALTLADAISDLPPVTNYVANDVMDYNDAAPKTEFENFISLKRSE |
| | | TLLPAFGGDPTRRLFDHQPLVLGDDDLERVSYIPKQKGANYRDMPG |
| | | VLVHNNKAEINPRFRAKLKSGKNVVPAYAISFIKGKSKKPFGRLWG |
| | | DEIVNTVVTRAEPHNQCVIHPMQNRVLSVRENARLQGFPDCYKLCG |
| | | TIKEKYIQVGNAVAVPVGVALGYAFGMASQGLTDDEPVIKLPFKYP |
| | | ECMQAKDQI |
| 620 | Arabadopsis | MLSPAKCESEEAQAPLDLHSSSRSEPECLSLVLWCPNPEEAAPSST |
| | CMT2 | RELIKLPDNGEMSLRRSTTLNCNSPEENGGEGRVSQRKSSRGKSQP |
| | | LLMLTNGCQLRRSPRFRALHANFDNVCSVPVTKGGVSQRKFSRGKS |
| | | QPLLTLINGCQLRRSPRFRAVDGNFDSVCSVPVTGKFGSRKRKSNS |
| | | ALDKKESSDSEGLTFKDIAVIAKSLEMEIISECQYKNNVAEGRSRL |
| | | QDPAKRKVDSDTLLYSSINSSKQSLGSNKRMRRSQRFMKGTENEGE |
| | | ENLGKSKGKGMSLASCSFRRSTRLSGTVETGNTETLNRRKDCGPAL |
| | | CGAEQVRGTERLVQISKKDHCCEAMKKCEGDGLVSSKQELLVFPSG |
| | | CIKKTVNGCRDRTLGKPRSSGLNTDDIHTSSLKISKNDTSNGLTMT |
| | | TALVEQDAMESLLQGKTSACGAADKGKTREMHVNSTVIYLSDSDEP |
| | | SSIEYLNGDNLTQVESGSALSSGGNEGIVSLDLNNPTKSTKRKGKR |
| | | VTRTAVQEQNKRSICFFIGEPLSCEEAQERWRWRYELKERKSKSRG |
| | | QQSEDDEDKIVANVECHYSQAKVDGHTFSLGDFAYIKGEEEETHVG |
| | | QIVEFFKITDGESYFRVQWFYRATDTIMERQATNHDKRRLFYSTVM |
| | | NDNPVDCLISKVTVLQVSPRVGLKPNSIKSDYYFDMEYCVEYSTFQ |
| | | TLRNPKTSENKLECCADVVPTESTESILKKKSFSGELPVLDLYSGC |
| | | GGMSTGLSLGAKISGVDVVTKWAVDQNTAACKSLKLNHPNTQVRND |
| | | AAGDFLQLLKEWDKLCKRYVENNDQRTDTLRSVNSTKETSGSSSSS |
| | | DDDSDSEEYEVEKLVDICFGDHDKTGKNGLKFKVHWKGYRSDEDTW |
| | | ELAEELSNCQDAIREFVTSGFKSKILPLPGRVGVICGGPPCQGISG |
| | | YNRHRNVDSPLNDERNQQIIVFMDIVEYLKPSYVLMENVVDILRMD |
| | | KGSLGRYALSRLVNMRYQARLGIMTAGCYGLSQFRSRVEMWGAVPN |
| | | KNLPPFPLPTHDVIVRYGLPLEFERNVVAYAEGQPRKLEKALVLKD |
| | | AISDLPHVSNDEDREKLPYESLPKTDFQRYIRSTKRDLIGSAIDNC |
| | | NKRTMLLHDHRPFHINEDDYARVCQIPKRKGANFRDLPGLIVRNNT |
| | | VCRDPSMEPVILPSGKPLVPGYVFTFQQGKSKRPFARLWWDETVPT |
| | | VLTVPTCHSQALLHPEQDRVLTIRESARLQGFPDYFQFCGTIKERY |
| | | CQIGNAVAVSVSRALGYSLGMAFRGLARDEHLIKLPQNFSHSTYPQ |
| | | LQETIPH |
| 621 | Arabadopsis | MAPKRKRPATKDDTTKSIPKPKKRAPKRAKTVKEEPVTVVEEGEKH |
| | CMT3 | VARFLDEPIPESEAKSTWPDRYKPIEVQPPKASSRKKTKDDEKVEI |
| | | IRARCHYRRAIVDERQIYELNDDAYVQSGEGKDPFICKIIEMFEGA |
| | | NGKLYFTARWFYRPSDTVMKEFEILIKKKRVFFSEIQDTNELGLLE |
| | | KKLNILMIPLNENTKETIPATENCDFFCDMNYFLPYDTFEAIQQET |
| | | MMAISESSTISSDTDIREGAAAISEIGECSQETEGHKKATLLDLYS |
| | | GCGAMSTGLCMGAQLSGLNLVTKWAVDMNAHACKSLQHNHPETNVR |
| | | NMTAEDFLFLLKEWEKLCIHFSLRNSPNSEEYANLHGLNNVEDNED |
| | | VSEESENEDDGEVFTVDKIVGISFGVPKKLLKRGLYLKVRWLNYDD |
| | | SHDTWEPIEGLSNCRGKIEEFVKLGYKSGILPLPGGVDVVCGGPPC |
| | | QGISGHNRFRNLLDPLEDQKNKQLLVYMNIVEYLKPKFVLMENVVD |
| | | MLKMAKGYLARFAVGRLLQMNYQVRNGMMAAGAYGLAQFRLRFFLW |
| | | GALPSEIIPQFPLPTHDLVHRGNIVKEFQGNIVAYDEGHTVKLADK |
| | | LLLKDVISDLPAVANSEKRDEITYDKDPTTPFQKFIRLRKDEASGS |
| | | QSKSKSKKHVLYDHHPLNLNINDYERVCQVPKRKGANFRDFPGVIV |
| | | GPGNVVKLEEGKERVKLESGKTLVPDYALTYVDGKSCKPFGRLWWD |
| | | EIVPTVVTRAEPHNQVIIHPEQNRVLSIRENARLQGFPDDYKLFGP |
| | | PKQKYIQVGNAVAVPVAKALGYALGTAFQGLAVGKDPLLTLPEGFA |
| | | FMKPTLPSELA |
| 622 | Neurospora Rid | MAEQNPFVIDDEDDVIQIHDEEEVEEEVAEVIDITEDDIEPSELDR |
| | | AFGSRPKEETLPSLLLRDQGFIVRPGMTVELKAPIGRFAISFVRVN |
| | | SIVKVRQAHVNNVTIRGHGFTRAKEMNGMLPKQLNECCLVASIDTR |
| | | DPRP |
| 623 | E. coli strain 12 | MNNNDLVAKLWKLCDNLRDGGVSYQNYVNELASLLFLKMCKETGQE |
| | hsdM | AEYLPEGYRWDDLKSRIGQEQLQFYRKMLVHLGEDDKKLVQAVFHN |
| | | VSTTITEPKQITALVSNMDSLDWYNGAHGKSRDDFGDMYEGLLQKN |
| | | ANETKSGAGQYFTPRPLIKTIIHLLKPQPREVVQDPAAGTAGFLIE |
| | | ADRYVKSQTNDLDDLDGDTQDFQIHRAFIGLELVPGTRRLALMNCL |
| | | LHDIEGNLDHGGAIRLGNTLGSDGENLPKAHIVATNPPFGSAAGTN |
| | | ITRTFVHPTSNKQLCFMQHIIETLHPGGRAAVVVPDNVLFEGGKGT |
| | | DIRRDLMDKCHLHTILRLPTGIFYAQGVKTNVLFFTKGTVANPNQD |
| | | KNCTDDVWVYDLRTNMPSFGKRTPFTDEHLQPFERVYGEDPHGLSP |
| | | RTEGEWSFNAEETEVADSEENKNTDQHLATSRWRKFSREWIRTAKS |
| | | DSLDISWLKDKDSIDADSLPEPDVLAAEAMGELVQALSELDALMRE |
| | | LGASDEADLQRQLLEEAFGGVKE |
| 624 | E. coli strain 12 | MSAGKLPEGWVIAPVSTVTTLIRGVTYKKEQAINYLKDDYLPLIRA |
| | hsdS | NNIQNGKFDTTDLVFVPKNLVKESQKISPEDIVIAMSSGSKSVVGK |
| | | SAHQHLPFECSFGAFCGVLRPEKLIFSGFIAHFTKSSLYRNKISSL |
| | | SAGANINNIKPASFDLINIPIPPLAEQKIIAEKLDTLLAQVDSTKA |
| | | RFEQIPQILKRFRQAVLGGAVNGKLTEKWRNFEPQHSVEKKLNFES |
| | | ILTELRNGLSSKPNESGVGHPILRISSVRAGHVDQNDIRFLECSES |
| | | ELNRHKLQDGDLLFTRYNGSLEFVGVCGLLKKLQHQNLLYPDKLIR |
| | | ARLTKDALPEYIEIFFSSPSARNAMMNCVKTTSGQKGISGKDIKSQ |
| | | VVLLPPVKEQAEIVRRVEQLFAYADTIEKQVNNALARVNNLTQSIL |
| | | AKAFRGELTAQWRAENPDLISGENSAAALLEKIKAERAASGGKKAS |
| | | RKKS |
| 625 | T. aquaticus M | MGLPPLLSLPSNSAPRSLGRVETPPEVVDFMVSLAEAPRGGRVLEP |
| | TaqI | ACAHGPFLRAFREAHGTAYRFVGVEIDPKALDLPPWAEGILADELL |
| | | WEPGEAFDLILGNPPYGIVGEASKYPIHVFKAVKDLYKKAFSTWKG |
| | | KYNLYGAFLEKAVRLLKPGGVLVFVVPATWLVLEDFALLREFLARE |
| | | GKTSVYYLGEVFPQKKVSAVVIRFQKSGKGLSLWDTQESESGFTPI |
| | | LWAEYPHWEGEIIRFETEETRKLEISGMPLGDLFHIRFAARSPEFK |
| | | KHPAVRKEPGPGLVPVLTGRNLKPGWVDYEKNHSGLWMPKERAKEL |
| | | RDFYATPHLVVAHTKGTRVVAAWDERAYPWREEFHLLPKEGVRLDP |
| | | SSLVQWLNSEAMQKHVRTLYRDFVPHLTLRMLERLPVRREYGFHTS |
| | | PESARNF |
| 626 | E. coli M EcoDam | MKKNRAFLKWAGGKYPLLDDIKRHLPKGECLVEPFVGAGSVFLNTD |
| | | FSRYILADINSDLISLYNIVKMRTDEYVQAARELFVPETNCAEVYY |
| | | QFREEFNKSQDPFRRAVLFLYLNRYGYNGLCRYNLRGEFNVPFGRY |
| | | KKPYFPEAELYHFAEKAQNAFFYCESYADSMARADDASVVYCDPPY |
| | | APLSATANFTAYHTNSFTLEQQAHLAEIAEGLVERHIPVLISNHDT |
| | | MLTREWYQRAKLHVVKVRRSISSNGGTRKKVDELLALYKPGVVSPA |
| | | KK |
| 627 | C. crescentus M | MKFGPETIIHGDCIEQMNALPEKSVDLIFADPPYNLQLGGDLLRPD |
| | CcrMI | NSKVDAVDDHWDQFESFAAYDKFTREWLKAARRVLKDDGAIWVIGS |
| | | YHNIFRVGVAVQDLGFWILNDIVWRKSNPMPNFKGTRFANAHETLI |
| | | WASKSQNAKRYTFNYDALKMANDEVQMRSDWTIPLCTGEERIKGAD |
| | | GQKAHPTQKPEALLYRVILSTTKPGDVILDPFFGVGTTGAAAKRLG |
| | | RKFIGIEREAEYLEHAKARIAKVVPIAPEDLDVMGSKRAEPRVPFG |
| | | TIVEAGLLSPGDTLYCSKGTHVAKVRPDGSITVGDLSGSIHKIGAL |
| | | VQSAPACNGWTYWHFKTDAGLAPIDVLRAQVRAGMN |
| 628 | C. difficile CamA | MDDISQDNFLLSKEYENSLDVDTKKASGIYYTPKIIVDYIVKKTLK |
| | | NHDIIKNPYPRILDISCGCGNFLLEVYDILYDLFEENIYELKKKYD |
| | | ENYWTVDNIHRHILNYCIYGADIDEKAISILKDSLINKKVVNDLDE |
| | | SDIKINLFCCDSLKKKWRYKFDYIVGNPPYIGHKKLEKKYKKFLLE |
| | | KYSEVYKDKADLYFCFYKKIIDILKQGGIGSVITPRYFLESLSGKD |
| | | LREYIKSNVNVQEIVDFLGANIFKNIGVSSCILTFDKKKTKETYID |
| | | VFKIKNEDICINKFETLEELLKSSKFEHFNINQRLLSDEWILVNKD |
| | | DETFYNKIQEKCKYSLEDIAISFQGIITGCDKAFILSKDDVKLNLV |
| | | DDKFLKCWIKSKNINKYIVDKSEYRLIYSNDIDNENINKRILDEII |
| | | GLYKTKLENRRECKSGIRKWYELQWGREKLFFERKKIMYPYKSNEN |
| | | RFAIDYDNNFSSADVYSFFIKEEYLDKFSYEYLVGILNSSVYDKYF |
| | | KITAKKMSKNIYDYYPNKVMKIRIFRDNNYEEIENLSKQIISILLN |
| | | KSIDKGKVEKLQIKMDNLIMDSLGI |
| 629 | KAP1 | MAASAAAASAAAASAASGSPGPGEGSAGGEKRSTAPSAAASASASA |
| | | AASSPAGGGAEALELLEHCGVCRERLRPEREPRLLPCLHSACSACL |
| | | GPAAPAAANSSGDGGAAGDGTVVDCPVCKQQCFSKDIVENYFMRDS |
| | | GSKAATDAQDANQCCTSCEDNAPATSYCVECSEPLCETCVEAHQRV |
| | | KYTKDHTVRSTGPAKSRDGERTVYCNVHKHEPLVLFCESCDTLTCR |
| | | DCQLNAHKDHQYQFLEDAVRNQRKLLASLVKRLGDKHATLQKSTKE |
| | | VRSSIRQVSDVQKRVQVDVKMAILQIMKELNKRGRVLVNDAQKVTE |
| | | GQQERLERQHWTMTKIQKHQEHILRFASWALESDNNTALLLSKKLI |
| | | YFQLHRALKMIVDPVEPHGEMKFQWDLNAWTKSAEAFGKIVAERPG |
| | | TNSTGPAPMAPPRAPGPLSKQGSGSSQPMEVQEGYGFGSGDDPYSS |
| | | AEPHVSGVKRSRSGEGEVSGLMRKVPRVSLERLDLDLTADSQPPVF |
| | | KVFPGSTTEDYNLIVIERGAAAAATGQPGTAPAGTPGAPPLAGMAI |
| | | VKEEETEAAIGAPPTATEGPETKPVLMALAEGPGAEGPRLASPSGS |
| | | TSSGLEVVAPEGTSAPGGGPGTLDDSATICRVCQKPGDLVMCNQCE |
| | | FCFHLDCHLPALQDVPGEEWSCSLCHVLPDLKEEDGSLSLDGADST |
| | | GVVAKLSPANQRKCERVLLALFCHEPCRPLHQLATDSTFSLDQPGG |
| | | TLDLTLIRARLQEKLSPPYSSPQEFAQDVGRMFKQFNKLTEDKADV |
| | | QSIIGLQRFFETRMNEAFGDTKFSAVLVEPPPMSLPGAGLSSQELS |
| | | GGPGDGP |
| 630 | MECP2 | MVAGMLGLREEKSEDQDLQGLKDKPLKFKKVKKDKKEEKEGKHEPV |
| | | QPSAHHSAEPAEAGKAETSEGSGSAPAVPEASASPKQRRSIIRDRG |
| | | PMYDDPTLPEGWTRKLKQRKSGRSAGKYDVYLINPQGKAFRSKVEL |
| | | IAYFEKVGDTSLDPNDFDFTVTGRGSPSRREQKPPKKPKSPKAPGT |
| | | GRGRGRPKGSGTTRPKAATSEGVQVKRVLEKSPGKLLVKMPFQTSP |
| | | GGKAEGGGATTSTQVMVIKRPGRKRKAEADPQAIPKKRGRKPGSVV |
| | | AAAAAEAKKKAVKESSIRSVQETVLPIKKRKTRETVSIEVKEVVKP |
| | | LLVSTLGEKSGKGLKICKSPGRKSKESSPKGRSSSASSPPKKEHHH |
| | | HHHHSESPKAPVPLLPPLPPPPPEPESSEDPTSPPEPQDLSSSVCK |
| | | EEKMPRGGSLESDGCPKEPAKTQPAVATAATAAEKYKHRGEGERKD |
| | | IVSSSMPRPNREEPVDSRTPVTERVS |
| 631 | linker | SGGS |
| 632 | linker | SGGSSGSETPGTSESATPESSGGS |
| 633 | linker | SGGSSGGSSGSETPGTSESATPESSGGSSGGS |
| 634 | linker | GGSGGSPGSPAGSPTSTEEGTSESATPESGPGTSTEPSEGSAPGSP |
| | | AGSPTSTEEGTSTEPSEGSAPGTSTEPSEGSAPGTSESATPESGPG |
| | | SEPATSGGSGGS |
| 635 | G linker | GSGGG |
| 636 | GX4 linker | GGGGSGGGGSGGGGSGGGGS |
| 637 | W linker | SSGNSNANSRGPSFSSGLVPLSLRGSH |
| 638 | XTEN linker | SGSETPGTSESATPES |
| | (XTEN16) | |
| 639 | XTEN linker | SGGSSGGSSGSETPGTSESATPES |
| 640 | XTEN linker | SGGSSGGSSGSETPGTSESATPESSGGSSGGSSGGSSGGS |
| 641 | XTEN linker | SGGSSGGSSGSETPGTSESATPESSGGSSGGSSGGSSGGSSGSETP |
| | | GTSESATPESSGGSSGGS |
| 642 | XTEN linker | PGSPAGSPTSTEEGTSESATPESGPGTSTEPSEGSAPGSPAGSPTS |
| | | TEEGTSTEPSEGSAPGTSTEPSEGSAPGTSESATPESGPGSEPATS |
| 643 | XTEN linker | GGPSSGAPPPSGGSPAGSPTSTEEGTSESATPESGPGTSTEPSEGS |
| | (XTEN80) | APGSPAGSPTSTEEGTSTEPSEGSAPGTSTEPSE |
| 644 | NLS | PKKKRKV |
| 645 | NLS | AVKRPAATKKAGQAKKKKLD |
| 546 | NLS | MSRRRKANPTKLSENAKKLAKEVEN |
| 647 | NLS | PAAKRVKLD |
| 648 | NLS | KLKIKRPVK |
| 649 | NLS | MDSLLMNRRKFLYQFKNVRWAKGRRETYLC |
| 660 | fusion protein | MGTMPKKKRKVPKKKRKVYNHDQEFDPPKVYPPVPAEKRKPIRVLS |
| | (Configuration 7) | LFDGIATGLLVLKDLGIQVDRYIASEVCEDSITVGMVRHQGKIMYV |
| | | GDVRSVTQKHIQEWGPFDLVIGGSPCNDLSIVNPARKGLYEGTGRL |
| | | FFEFYRLLHDARPKEGDDRPFFWLFENVVAMGVSDKRDISRFLESN |
| | | PVMIDAKEVSAAHRARYFWGNLPGMNRPLASTVNDKLELQECLEHG |
| | | RIAKFSKVRTITTRSNSIKQGKDQHFPVFMNEKEDILWCTEMERVF |
| | | GFPVHYTDVSNMSRLARQRLLGRSWSVPVIRHLFAPLKEYFACVSS |
| | | GNSNANSRGPSFSSGLVPLSLRGSHMAAIPALDPEAEPSMDVILVG |
| | | SSELSSSVSPGTGRDLIAYEVKANQRNIEDICICCGSLQVHTQHPL |
| | | FEGGICAPCKDKFLDALFLYDDDGYQSYCSICCSGETLLICGNPDC |
| | | TRCYCFECVDSLVGPGTSGKVHAMSNWVCYLCLPSSRSGLLQRRRK |
| | | WRSQLKAFYDRESENPLEMFETVPVWRRQPVRVLSLFEDIKKELTS |
| | | LGFLESGSDPGQLKHVVDVTDTVRKDVEEWGPFDLVYGATPPLGHT |
| | | CDRPPSWYLFQFHRLLQYARPKPGSPRPFFWMFVDNLVLNKEDLDV |
| | | ASRFLEMEPVTIPDVHGGSLQNAVRVWSNIPAIRSRHWALVSEEEL |
| | | SLLAQNKQSSKLAAKWPTKLVKNCFLPLREYFKYFSTELTSSLGGP |
| | | SSGAPPPSGGSPAGSPTSTEEGTSESATPESGPGTSTEPSEGSAPG |
| | | SPAGSPTSTEEGTSTEPSEGSAPGTSTEPSELEDKKYSIGLAIGTN |
| | | SVGWAVITDEYKVPSKKFKVLGNTDRHSIKKNLIGALLFDSGETAE |
| | | ATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLR |
| | | LIYLALAHMIKFRGHFLIEGDLNPDNSDVDKLFIQLVQTYNQLFEE |
| | | NPINASGVDAKAILSARLSKSRRLENLIAQLPGEKKNGLEGNLIAL |
| | | SLGLTPNFKSNFDLAEDAKLQLSKDTYDDDLDNLLAQIGDQYADLF |
| | | LAAKNLSDAILLSDILRVNTEITKAPLSASMIKRYDEHHQDLTLLK |
| | | ALVRQQLPEKYKEIFFDQSKNGYAGYIDGGASQEEFYKFIKPILEK |
| | | MDGTEELLVKLNREDLLRKQRTFDNGSIPHQIHLGELHAILRRQED |
| | | FYPFLKDNREKIEKILTFRIPYYVGPLARGNSRFAWMTRKSEETIT |
| | | PWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKE |
| | | DYFKKIECFDSVEISGVEDRFNASLGTYHDLLKIIKDKDFLDNEEN |
| | | EDILEDIVLTLTLFEDREMIEERLKTYAHLEDDKVMKQLKRRRYTG |
| | | WGRLSRKLINGIRDKQSGKTILDFLKSDGFANRNFMQLIHDDSLTF |
| | | KEDIQKAQVSGQGDSLHEHIANLAGSPAIKKGILQTVKVVDELVKV |
| | | MGRHKPENIVIEMARENQTTQKGQKNSRERMKRIEEGIKELGSQIL |
| | | KEHPVENTQLQNEKLYLYYLQNGRDMYVDQELDINRLSDYDVDAIV |
| | | PQSFLKDDSIDNKVLTRSDKNRGKSDNVPSEEVVKKMKNYWRQLLN |
| | | AKLITQRKFDNLTKAERGGLSELDKAGFIKRQLVETRQITKHVAQI |
| | | LDSRMNTKYDENDKLIREVKVITLKSKLVSDFRKDFQFYKVREINN |
| | | YHHAHDAYLNAVVGTALIKKYPKLESEFVYGDYKVYDVRKMIAKSE |
| | | QEIGKATAKYFFYSNIMNFFKTEITLANGEIRKRPLIEINGETGEI |
| | | VWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELL |
| | | GITIMERSSFEKNPIDFLEAKGYKEVKKDLIIKLPKYSLFELENGR |
| | | KRMLASAGELQKGNELALPSKYVNFLYLASHYEKLKGSPEDNEQKQ |
| | | LFVEQHKHYLDEIIEQISEFSKRVILADANLDKVLSAYNKHRDKPI |
| | | REQAENIIHLFTLTNLGAPAAFKYFDTTIDRKRYTSTKEVLDATLI |
| | | HQSITGLYETRIDLSQLGGDSPKKKRKVGVDGSSGSETPGTSESAT |
| | | PESRTLVTFKDVFVDFTREEWKLLDTAQQIVYRNVMLENYKNLVSL |
| | | GYQLTKPDVILRLEKGEEPSADYKDDDDKAPKKKRKVPKKKRKV |
| 661 | fusion protein | MGTMPKKKRKVPKKKRKVYNHDQEFDPPKVYPPVPAEKRKPIRVLS |
| | (Configuration 9) | LFDGIATGLLVLKDLGIQVDRYIASEVCEDSITVGMVRHQGKIMYV |
| | | GDVRSVTQKHIQEWGPFDLVIGGSPCNDLSIVNPARKGLYEGTGRL |
| | | FFEFYRLLHDARPKEGDDRPFFWLFENVVAMGVSDKRDISRFLESN |
| | | PVMIDAKEVSAAHRARYFWGNLPGMNRPLASTVNDKLELQECLEHG |
| | | RIAKFSKVRTITTRSNSIKQGKDQHFPVFMNEKEDILWCTEMERVF |
| | | GFPVHYTDVSNMSRLARQRLLGRSWSVPVIRHLFAPLKEYFACVSS |
| | | GNSNANSRGPSFSSGLVPLSLRGSHMAAIPALDPEAEPSMDVILVG |
| | | SSELSSSVSPGTGRDLIAYEVKANQRNIEDICICCGSLQVHTQHPL |
| | | FEGGICAPCKDKFLDALFLYDDDGYQSYCSICCSGETLLICGNPDC |
| | | TRCYCFECVDSLVGPGTSGKVHAMSNWVCYLCLPSSRSGLLQRRRK |
| | | WRSQLKAFYDRESENPLEMFETVPVWRRQPVRVLSLFEDIKKELTS |
| | | LGFLESGSDPGQLKHVVDVTDTVRKDVEEWGPFDLVYGATPPLGHT |
| | | CDRPPSWYLFQFHRLLQYARPKPGSPRPFFWMFVDNLVLNKEDLDV |
| | | ASRFLEMEPVTIPDVHGGSLQNAVRVWSNIPAIRSRHWALVSEEEL |
| | | SLLAQNKQSSKLAAKWPTKLVKNCFLPLREYFKYFSTELTSSLGGP |
| | | SSGAPPPSGGSPAGSPTSTEEGTSESATPESGPGTSTEPSEGSAPG |
| | | SPAGSPTSTEEGTSTEPSEGSAPGTSTEPSELEDKKYSIGLAIGTN |
| | | SVGWAVITDEYKVPSKKFKVLGNTDRHSIKKNLIGALLFDSGETAE |
| | | ATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLR |
| | | LIYLALAHMIKFRGHFLIEGDLNPDNSDVDKLFIQLVQTYNQLFEE |
| | | NPINASGVDAKAILSARLSKSRRLENLIAQLPGEKKNGLFGNLIAL |
| | | SLGLTPNFKSNFDLAEDAKLQLSKDTYDDDLDNLLAQIGDQYADLF |
| | | LAAKNLSDAILLSDILRVNTEITKAPLSASMIKRYDEHHQDLTLLK |
| | | ALVRQQLPEKYKEIFFDQSKNGYAGYIDGGASQEEFYKFIKPILEK |
| | | MDGTEELLVKLNREDLLRKQRTFDNGSIPHQIHLGELHAILRRQED |
| | | FYPFLKDNREKIEKILTFRIPYYVGPLARGNSRFAWMTRKSEETIT |
| | | PWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKE |
| | | DYFKKIECFDSVEISGVEDRFNASLGTYHDLLKIIKDKDELDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTF |
| | | KEDIQKAQVSGQGDSLHEHIANLAGSPAIKKGILQTVKVVDELVKV |
| | | MGRHKPENIVIEMARENQTTQKGQKNSRERMKRIEEGIKELGSQIL |
| | | KEHPVENTQLQNEKLYLYYLQNGRDMYVDQELDINRLSDYDVDAIV |
| | | PQSFLKDDSIDNKVLTRSDKNRGKSDNVPSEEVVKKMKNYWRQLLN |
| | | AKLITQRKFDNLTKAERGGLSELDKAGFIKRQLVETRQITKHVAQI |
| | | LDSRMNTKYDENDKLIREVKVITLKSKLVSDFRKDFQFYKVREINN |
| | | YHHAHDAYLNAVVGTALIKKYPKLESEFVYGDYKVYDVRKMIAKSE |
| | | QEIGKATAKYFFYSNIMNFFKTEITLANGEIRKRPLIETNGETGEI |
| | | VWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELL |
| | | GITIMERSSFEKNPIDFLEAKGYKEVKKDLIIKLPKYSLFELENGR |
| | | KRMLASAGELQKGNELALPSKYVNFLYLASHYEKLKGSPEDNEQKQ |
| | | LFVEQHKHYLDEIIEQISEFSKRVILADANLDKVLSAYNKHRDKPI |
| | | REQAENIIHLFTLTNLGAPAAFKYFDTTIDRKRYTSTKEVLDATLI |
| | | HQSITGLYETRIDLSQLGGDSPKKKRKVGVDGSSGSETPGTSESAT |
| | | PESTGNKKLEAVGTGIEPKAMSQGLVTFGDVAVDFSQEEWEWLNPI |
| | | QRNLYRKVMLENYRNLASLGLCVSKPDVISSLEQGKEPWSADYKDD |
| | | DDKAPKKKRKVPKKKRKV |
| 662 | fusion protein | MGTMPKKKRKVPKKKRKVYNHDQEFDPPKVYPPVPAEKRKPIRVLS |
| | (Configuration 11) | LFDGIATGLLVLKDLGIQVDRYIASEVCEDSITVGMVRHQGKIMYV |
| | | GDVRSVTQKHIQEWGPFDLVIGGSPCNDLSIVNPARKGLYEGTGRL |
| | | FFEFYRLLHDARPKEGDDRPFFWLFENVVAMGVSDKRDISRFLESN |
| | | PVMIDAKEVSAAHRARYFWGNLPGMNRPLASTVNDKLELQECLEHG |
| | | RIAKFSKVRTITTRSNSIKQGKDQHFPVFMNEKEDILWCTEMERVF |
| | | GFPVHYTDVSNMSRLARQRLLGRSWSVPVIRHLFAPLKEYFACVSS |
| | | GNSNANSRGPSFSSGLVPLSLRGSHMAAIPALDPEAEPSMDVILVG |
| | | SSELSSSVSPGTGRDLIAYEVKANQRNIEDICICCGSLQVHTQHPL |
| | | FEGGICAPCKDKFLDALFLYDDDGYQSYCSICCSGETLLICGNPDC |
| | | TRCYCFECVDSLVGPGTSGKVHAMSNWVCYLCLPSSRSGLLQRRRK |
| | | WRSQLKAFYDRESENPLEMFETVPVWRRQPVRVLSLFEDIKKELTS |
| | | LGFLESGSDPGQLKHVVDVTDTVRKDVEEWGPFDLVYGATPPLGHT |
| | | CDRPPSWYLFQFHRLLQYARPKPGSPRPFFWMFVDNLVLNKEDLDV |
| | | ASRFLEMEPVTIPDVHGGSLQNAVRVWSNIPAIRSRHWALVSEEEL |
| | | SLLAQNKQSSKLAAKWPTKLVKNCFLPLREYFKYFSTELTSSLGGP |
| | | SSGAPPPSGGSPAGSPTSTEEGTSESATPESGPGTSTEPSEGSAPG |
| | | SPAGSPTSTEEGTSTEPSEGSAPGTSTEPSELEDKKYSIGLAIGTN |
| | | SVGWAVITDEYKVPSKKFKVLGNTDRHSIKKNLIGALLFDSGETAE |
| | | ATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLR |
| | | LIYLALAHMIKFRGHFLIEGDLNPDNSDVDKLFIQLVQTYNQLFEE |
| | | NPINASGVDAKAILSARLSKSRRLENLIAQLPGEKKNGLFGNLIAL |
| | | SLGLTPNFKSNFDLAEDAKLQLSKDTYDDDLDNLLAQIGDQYADLF |
| | | LAAKNLSDAILLSDILRVNTEITKAPLSASMIKRYDEHHQDLTLLK |
| | | ALVRQQLPEKYKEIFFDQSKNGYAGYIDGGASQEEFYKFIKPILEK |
| | | MDGTEELLVKLNREDLLRKQRTFDNGSIPHQIHLGELHAILRRQED |
| | | FYPFLKDNREKIEKILTFRIPYYVGPLARGNSRFAWMTRKSEETIT |
| | | PWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKE |
| | | DYFKKIECFDSVEISGVEDRFNASLGTYHDLLKIIKDKDFLDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTF |
| | | KEDIQKAQVSGQGDSLHEHIANLAGSPAIKKGILQTVKVVDELVKV |
| | | MGRHKPENIVIEMARENQTTQKGQKNSRERMKRIEEGIKELGSQIL |
| | | KEHPVENTQLQNEKLYLYYLQNGRDMYVDQELDINRLSDYDVDAIV |
| | | PQSFLKDDSIDNKVLTRSDKNRGKSDNVPSEEVVKKMKNYWRQLLN |
| | | AKLITQRKFDNLTKAERGGLSELDKAGFIKRQLVETRQITKHVAQI |
| | | LDSRMNTKYDENDKLIREVKVITLKSKLVSDFRKDFQFYKVREINN |
| | | YHHAHDAYLNAVVGTALIKKYPKLESEFVYGDYKVYDVRKMIAKSE |
| | | QEIGKATAKYFFYSNIMNFFKTEITLANGEIRKRPLIETNGETGEI |
| | | VWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELL |
| | | GITIMERSSFEKNPIDFLEAKGYKEVKKDLIIKLPKYSLFELENGR |
| | | KRMLASAGELQKGNELALPSKYVNFLYLASHYEKLKGSPEDNEQKQ |
| | | LFVEQHKHYLDEIIEQISEFSKRVILADANLDKVLSAYNKHRDKPI |
| | | REQAENIIHLFTLTNLGAPAAFKYFDTTIDRKRYTSTKEVLDATLI |
| | | HQSITGLYETRIDLSQLGGDSPKKKRKVGVDGSSGSETPGTSESAT |
| | | PESTGDSVAFEDVAVNFTLEEWALLDPSQKNLYRDVMRETERNLAS |
| | | VGKQWEDQNIEDPFKIPRRNISHIPERLCESKEGGQGEESADYKDD |
| | | DDKAPKKKRKVPKKKRKV |
| 663 | fusion protein | MGTMPKKKRKVPKKKRKVYNHDQEFDPPKVYPPVPAEKRKPIRVLS |
| | (Configuration 13) | LFDGIATGLLVLKDLGIQVDRYIASEVCEDSITVGMVRHQGKIMYV |
| | | GDVRSVTQKHIQEWGPFDLVIGGSPCNDLSIVNPARKGLYEGTGRL |
| | | FFEFYRLLHDARPKEGDDRPFFWLFENVVAMGVSDKRDISRFLESN |
| | | PVMIDAKEVSAAHRARYFWGNLPGMNRPLASTVNDKLELQECLEHG |
| | | RIAKFSKVRTITTRSNSIKQGKDQHFPVFMNEKEDILWCTEMERVF |
| | | GFPVHYTDVSNMSRLARQRLLGRSWSVPVIRHLFAPLKEYFACVSS |
| | | GNSNANSRGPSFSSGLVPLSLRGSHMAAIPALDPEAEPSMDVILVG |
| | | SSELSSSVSPGTGRDLIAYEVKANQRNIEDICICCGSLQVHTQHPL |
| | | FEGGICAPCKDKFLDALFLYDDDGYQSYCSICCSGETLLICGNPDC |
| | | TRCYCFECVDSLVGPGTSGKVHAMSNWVCYLCLPSSRSGLLQRRRK |
| | | WRSQLKAFYDRESENPLEMFETVPVWRRQPVRVLSLFEDIKKELTS |
| | | LGFLESGSDPGQLKHVVDVTDTVRKDVEEWGPFDLVYGATPPLGHT |
| | | CDRPPSWYLFQFHRLLQYARPKPGSPRPFFWMFVDNLVLNKEDLDV |
| | | ASRFLEMEPVTIPDVHGGSLQNAVRVWSNIPAIRSRHWALVSEEEL |
| | | SLLAQNKQSSKLAAKWPTKLVKNCFLPLREYFKYFSTELTSSLGGP |
| | | SSGAPPPSGGSPAGSPTSTEEGTSESATPESGPGTSTEPSEGSAPG |
| | | SPAGSPTSTEEGTSTEPSEGSAPGTSTEPSELEDKKYSIGLAIGTN |
| | | SVGWAVITDEYKVPSKKFKVLGNTDRHSIKKNLIGALLFDSGETAE |
| | | ATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLR |
| | | LIYLALAHMIKFRGHFLIEGDLNPDNSDVDKLFIQLVQTYNQLFEE |
| | | NPINASGVDAKAILSARLSKSRRLENLIAQLPGEKKNGLFGNLIAL |
| | | SLGLTPNFKSNEDLAEDAKLQLSKDTYDDDLDNLLAQIGDQYADLF |
| | | LAAKNLSDAILLSDILRVNTEITKAPLSASMIKRYDEHHQDLTLLK |
| | | ALVRQQLPEKYKEIFFDQSKNGYAGYIDGGASQEEFYKFIKPILEK |
| | | MDGTEELLVKLNREDLLRKQRTFDNGSIPHQIHLGELHAILRRQED |
| | | FYPFLKDNREKIEKILTFRIPYYVGPLARGNSRFAWMTRKSEETIT |
| | | PWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKE |
| | | DYFKKIECFDSVEISGVEDRFNASLGTYHDLLKIIKDKDFLDNEEN |
| | | EDILEDIVLILTLFEDREMIEERLKTYAHLEDDKVMKQLKRRRYTG |
| | | WGRLSRKLINGIRDKQSGKTILDFLKSDGFANRNFMQLIHDDSLTF |
| | | KEDIQKAQVSGQGDSLHEHIANLAGSPAIKKGILQTVKVVDELVKV |
| | | MGRHKPENIVIEMARENQTTQKGQKNSRERMKRIEEGIKELGSQIL |
| | | KEHPVENTQLQNEKLYLYYLQNGRDMYVDQELDINRLSDYDVDAIV |
| | | PQSFLKDDSIDNKVLTRSDKNRGKSDNVPSEEVVKKMKNYWRQLLN |
| | | AKLITQRKFDNLTKAERGGLSELDKAGFIKRQLVETRQITKHVAQI |
| | | LDSRMNTKYDENDKLIREVKVITLKSKLVSDFRKDFQFYKVREINN |
| | | YHHAHDAYLNAVVGTALIKKYPKLESEFVYGDYKVYDVRKMIAKSE |
| | | QEIGKATAKYFFYSNIMNFFKTEITLANGEIRKRPLIETNGETGEI |
| | | VWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELL |
| | | GITIMERSSFEKNPIDFLEAKGYKEVKKDLIIKLPKYSLFELENGR |
| | | KRMLASAGELQKGNELALPSKYVNFLYLASHYEKLKGSPEDNEQKQ |
| | | LFVEQHKHYLDEIIEQISEFSKRVILADANLDKVLSAYNKHRDKPI |
| | | REQAENIIHLFTLTNLGAPAAFKYFDTTIDRKRYTSTKEVLDATLI |
| | | HQSITGLYETRIDLSQLGGDSPKKKRKVGVDGSSGSETPGTSESAT |
| | | PESTGMNNSQGRVTFEDVTVNFTQGEWQRLNPEQRNLYRDVMLENY |
| | | SNLVSVGQGETTKPDVILRLEQGKEPWLEEEEVLGSGRAEKNGDIG |
| | | GQIWKPKDVKESLSADYKDDDDKAPKKKRKVPKKKRKV |
| 664 | linker | GGGGS |
| 665 | linker | EAAAK |
| 666 | linker | SGGS |
| | WTCas9 | TCAACTGCGACCAGTTCAGCGTTTAAGAGCTAGAAATAGCAAGTTTAAATAAGGC |
| | RNA468 | TAGTCCGTTATCAACTTGAAAAAGTGGCACCGAGTCGGTGCTTTTTTT |
| | Fusion Protein 1 | MPKKKRKVPKKKRKVNHDQEFDPPKVYPPVPAEKRKPIRVLSLFDGIA |
| | Amino Acid | TGLLVLKDLGIQVDRYIASEVCEDSITVGMVRHQGKIMYVGDVRSVT |
| | Sequence | QKHIQEWGPFDLVIGGSPCNDLSIVNPARKGLYEGTGRLFFEFYRLLH |
| | NLS-NLS-3A-3L- | DARPKEGDDRPFFWLFENVVAMGVSDKRDISRFLESNPVMIDAKEVS |
| | dCas9-KRAB- | AAHRARYFWGNLPGMNRPLASTVNDKLELQECLEHGRIAKFSKVRTI |
| | NLS-NLS | TTRSNSIKQGKDQHFPVFMNEKEDILWCTEMERVFGFPVHYTDVSN |
| | | MSRLARQRLLGRSWSVPVIRHLFAPLKEYFACVSSGNSNANSRGPSFS |
| | | SGLVPLSLRGSHNPLEMFETVPVWRRQPVRVLSLFEDIKKELTSLGFLE |
| | | SGSDPGQLKHVVDVTDTVRKDVEEWGPFDLVYGATPPLGHTCDRPP |
| | | SWYLFQFHRLLQYARPKPGSPRPFFWMFVDNLVLNKEDLDVASRFLE |
| | | MEPVTIPDVHGGSLQNAVRVWSNIPAIRSRHWALVSEEELSLLAQNK |
| | | QSSKLAAKWPTKLVKNCFLPLREYFKYFSTELTSSLGGPSSGAPPPSGG |
| | | SPAGSPTSTEEGTSESATPESGPGTSTEPSEGSAPGSPAGSPTSTEEGT |
| | | STEPSEGSAPGTSTEPSEMDKKYSIGLAIGTNSVGWAVITDEYKVPSKK |
| | | FKVLGNTDRHSIKKNLIGALLFDSGETAEATRLKRTARRRYTRRKNRICY |
| | | LQEIFSNEMAKVDDSFFHRLEESFLVEEDKKHERHPIFGNIVDEVAYHE |
| | | KYPTIYHLRKKLVDSTDKADLRLIYLALAHMIKFRGHFLIEGDLNPDNSD |
| | | VDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLL |
| | | AQIGDQYADLFLAAKNLSDAILLSDILRVNTEITKAPLSASMIKRYDEHH |
| | | QDLTLLKALVRQQLPEKYKEIFFDQSKNGYAGYIDGGASQEEFYKFIKPI |
| | | LEKMDGTEELLVKLNREDLLRKQRTFDNGSIPHQIHLGELHAILRRQED |
| | | FYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECF |
| | | DSVEISGVEDRFNASLGTYHDLLKIIKDKDFLDNEENEDILEDIVLTLTLF |
| | | EDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSL |
| | | HEHIANLAGSPAIKKGILQTVKVVDELVKVMGRHKPENIVIEMARENQ |
| | | TTQKGQKNSRERMKRIEEGIKELGSQILKEHPVENTQLQNEKLYLYYLQ |
| | | NGRDMYVDQELDINRLSDYDVDAIVPQSFLKDDSIDNKVLTRSDKNR |
| | | GKSDNVPSEEVVKKMKNYWRQLLNAKLITQRKFDNLTKAERGGLSEL |
| | | DKAGFIKRQLVETRQITKHVAQILDSRMNTKYDENDKLIREVKVITLKS |
| | | KLVSDFRKDFQFYKVREINNYHHAHDAYLNAVVGTALIKKYPKLESEFV |
| | | YGDYKVYDVRKMIAKSEQEIGKATAKYFFYSNIMNFFKTEITLANGEIR |
| | | KRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKS |
| | | KKLKSVKELLGITIMERSSFEKNPIDFLEAKGYKEVKKDLIIKLPKYSLFELE |
| | | NGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIRE |
| | | QAENIIHLFTLTNLGAPAAFKYFDTTIDRKRYTSTKEVLDATLIHQSITGL |
| | | YETRIDLSQLGGDSGSETPGTSESATPESTGRTLVTFKDVFVDFTREEW |
| | | KLLDTAQQIVYRNVMLENYKNLVSLGYQLTKPDVILRLEKGEEPPKKKR |
| | | KVPKKKRKV |
| | Fusion Protein 1 | ATGCCAAAAAAGAAGAGAAAGGTACCGAAGAAAAAAAGAAAGGT |
| | DNA Sequence | CAACCATGATCAAGAATTCGACCCACCTAAAGTCTACCCACCTGTG |
| | | CCCGCCGAAAAAAGGAAACCCATAAGGGTGCTGTCACTCTTTGAT |
| | | GGCATCGCCACTGGTCTCCTGGTTCTTAAGGATCTGGGAATTCAGG |
| | | TCGATCGGTACATTGCTAGCGAGGTTTGTGAGGATAGTATTACAGT |
| | | GGGTATGGTGCGCCACCAGGGAAAGATCATGTATGTTGGTGACGT |
| | | TAGGAGCGTCACCCAGAAACATATCCAGGAGTGGGGACCCTTTGA |
| | | TTTGGTGATCGGAGGTAGTCCCTGCAATGACCTTTCCATCGTGAAT |
| | | CCAGCCAGGAAAGGGCTGTATGAAGGGACTGGTAGGCTCTTTTTC |
| | | GAGTTTTATCGCCTGCTTCACGACGCTAGACCTAAGGAAGGTGAC |
| | | GATAGGCCTTTCTTTTGGCTTTTTGAGAACGTCGTGGCAATGGGAG |
| | | TCTCCGACAAAAGGGACATTTCTCGCTTTCTGGAATCTAACCCCGTT |
| | | ATGATCGATGCCAAGGAAGTTTCTGCCGCTCACAGGGCAAGGTAC |
| | | TTCTGGGGCAATCTGCCCGGAATGAATCGCCCACTGGCCAGTACC |
| | | GTGAATGACAAACTGGAGCTGCAGGAGTGCCTGGAGCACGGAAG |
| | | AATCGCAAAGTTTTCTAAAGTCAGGACCATTACCACTCGCAGTAAC |
| | | TCCATAAAACAGGGTAAGGACCAGCATTTTCCCGTCTTCATGAATG |
| | | AAAAGGAAGATATTCTGTGGTGCACTGAAATGGAGAGAGTTTTCG |
| | | GGTTTCCCGTGCACTATACCGATGTTTCCAACATGTCCCGCCTTGCA |
| | | AGACAAAGGCTTTTGGGCCGCTCTTGGTCTGTGCCAGTGATCCGG |
| | | CACTTGTTTGCTCCCCTCAAAGAGTACTTCGCTTGCGTCAGTTCCGG |
| | | AAATTCAAACGCTAACTCTCGGGGTCCATCTTTCTCCAGTGGTCTC |
| | | GTGCCACTGTCTCTCCGGGGCTCTCACAATCCCCTGGAGATGTTTG |
| | | AGACAGTGCCAGTCTGGCGGAGGCAGCCCGTTCGCGTTCTCTCTCT |
| | | GTTCGAAGATATTAAAAAGGAACTCACCTCCCTTGGGTTCCTGGAG |
| | | AGCGGGAGCGACCCCGGACAGCTTAAGCACGTGGTCGACGTGAC |
| | | TGACACCGTCCGCAAAGACGTGGAGGAATGGGGCCCCTTCGATCT |
| | | GGTCTATGGGGCAACCCCTCCCCTTGGGCATACATGTGATCGGCCT |
| | | CCATCCTGGTACCTGTTCCAGTTTCACAGACTCCTGCAGTATGCCA |
| | | GGCCAAAGCCAGGGAGCCCAAGGCCCTTTTTCTGGATGTTCGTCG |
| | | ACAACCTGGTCCTGAACAAAGAAGATCTCGACGTTGCTAGTCGCTT |
| | | TCTCGAAATGGAGCCCGTGACCATTCCCGACGTGCATGGCGGTTCC |
| | | CTCCAGAATGCAGTCAGGGTTTGGAGCAATATCCCTGCCATCAGGT |
| | | CAAGGCACTGGGCACTGGTTTCAGAGGAAGAGCTGTCCCTCCTTG |
| | | CCCAGAACAAGCAGTCATCCAAACTGGCAGCCAAGTGGCCAACTA |
| | | AGCTGGTCAAGAACTGCTTTCTTCCCCTCAGAGAATATTTTAAGTAT |
| | | TTCAGTACTGAACTGACTAGCAGTCTGGGAGGGCCGAGCTCTGGC |
| | | GCACCCCCACCAAGTGGAGGGTCTCCTGCCGGGTCCCCAACATCTA |
| | | CTGAAGAAGGCACCAGCGAATCCGCAACGCCCGAGTCAGGCCCTG |
| | | GTACCTCCACAGAACCATCTGAAGGTAGTGCGCCTGGTTCCCCAGC |
| | | TGGAAGCCCTACTTCCACCGAAGAAGGCACGTCAACCGAACCAAG |
| | | TGAAGGATCTGCCCCTGGGACCAGCACTGAACCATCTGAGATGGA |
| | | CAAGAAGTACAGCATCGGCCTGGCCATCGGCACCAACTCTGTGGG |
| | | CTGGGCCGTGATCACCGACGAGTACAAGGTGCCCAGCAAGAAATT |
| | | CAAGGTGCTGGGCAACACCGACCGGCACAGCATCAAGAAGAACCT |
| | | GATCGGCGCCCTGCTGTTCGACAGCGGAGAAACAGCCGAGGCCAC |
| | | CCGGCTGAAGAGAACCGCCAGAAGAAGATACACCAGACGGAAGA |
| | | ACCGGATCTGCTATCTGCAAGAGATCTTCAGCAACGAGATGGCCA |
| | | AGGTGGACGACAGCTTCTTCCACAGACTGGAAGAGTCCTTCCTGG |
| | | TGGAAGAGGATAAGAAGCACGAGCGGCACCCCATCTTCGGCAACA |
| | | TCGTGGACGAGGTGGCCTACCACGAGAAGTACCCCACCATCTACC |
| | | ACCTGAGAAAGAAACTGGTGGACAGCACCGACAAGGCCGACCTG |
| | | CGGCTGATCTATCTGGCCCTGGCCCACATGATCAAGTTCCGGGGCC |
| | | ACTTCCTGATCGAGGGCGACCTGAACCCCGACAACAGCGACGTGG |
| | | ACAAGCTGTTCATCCAGCTGGTGCAGACCTACAACCAGCTGTTCGA |
| | | GGAAAACCCCATCAACGCCAGCGGCGTGGACGCCAAGGCCATCCT |
| | | GTCTGCCAGACTGAGCAAGAGCAGACGGCTGGAAAATCTGATCGC |
| | | CCAGCTGCCCGGCGAGAAGAAGAATGGCCTGTTCGGCAACCTGAT |
| | | TGCCCTGAGCCTGGGCCTGACCCCCAACTTCAAGAGCAACTTCGAC |
| | | CTGGCCGAGGATGCCAAACTGCAGCTGAGCAAGGACACCTACGAC |
| | | GACGACCTGGACAACCTGCTGGCCCAGATCGGCGACCAGTACGCC |
| | | GACCTGTTTCTGGCCGCCAAGAACCTGTCCGACGCCATCCTGCTGA |
| | | GCGACATCCTGAGAGTGAACACCGAGATCACCAAGGCCCCCCTGA |
| | | GCGCCTCTATGATCAAGAGATACGACGAGCACCACCAGGACCTGA |
| | | CCCTGCTGAAAGCTCTCGTGCGGCAGCAGCTGCCTGAGAAGTACA |
| | | AAGAGATTTTCTTCGACCAGAGCAAGAACGGCTACGCCGGCTACA |
| | | TCGATGGCGGAGCCAGCCAGGAAGAGTTCTACAAGTTCATCAAGC |
| | | CCATCCTGGAAAAGATGGACGGCACCGAGGAACTGCTCGTGAAGC |
| | | TGAACAGAGAGGACCTGCTGCGGAAGCAGCGGACCTTCGACAAC |
| | | GGCAGCATCCCCCACCAGATCCACCTGGGAGAGCTGCACGCCATT |
| | | CTGCGGCGGCAGGAAGATTTTTACCCATTCCTGAAGGACAACCGG |
| | | GAAAAGATCGAGAAGATCCTGACCTTCCGCATCCCCTACTACGTGG |
| | | GCCCTCTGGCCAGGGGAAACAGCAGATTCGCCTGGATGACCAGAA |
| | | AGAGCGAGGAAACCATCACCCCCTGGAACTTCGAGGAAGTGGTG |
| | | GACAAGGGCGCCAGCGCCCAGAGCTTCATCGAGCGGATGACCAAC |
| | | TTCGATAAGAACCTGCCCAACGAGAAGGTGCTGCCCAAGCACAGC |
| | | CTGCTGTACGAGTACTTCACCGTGTACAACGAGCTGACCAAAGTG |
| | | AAATACGTGACCGAGGGAATGAGAAAGCCCGCCTTCCTGAGCGGC |
| | | GAGCAGAAAAAAGCCATCGTGGACCTGCTGTTCAAGACCAACCGG |
| | | AAAGTGACCGTGAAGCAGCTGAAAGAGGACTACTTCAAGAAAATC |
| | | GAGTGCTTCGACTCCGTGGAAATCTCCGGCGTGGAAGATCGGTTC |
| | | AACGCCTCCCTGGGCACATACCACGATCTGCTGAAAATTATCAAGG |
| | | ACAAGGACTTCCTGGACAATGAGGAAAACGAGGACATTCTGGAAG |
| | | ATATCGTGCTGACCCTGACACTGTTTGAGGACAGAGAGATGATCG |
| | | AGGAACGGCTGAAAACCTATGCCCACCTGTTCGACGACAAAGTGA |
| | | TGAAGCAGCTGAAGCGGCGGAGATACACCGGCTGGGGCAGGCTG |
| | | AGCCGGAAGCTGATCAACGGCATCCGGGACAAGCAGTCCGGCAA |
| | | GACAATCCTGGATTTCCTGAAGTCCGACGGCTTCGCCAACAGAAAC |
| | | TTCATGCAGCTGATCCACGACGACAGCCTGACCTTTAAAGAGGAC |
| | | ATCCAGAAAGCCCAGGTGTCCGGCCAGGGCGATAGCCTGCACGA |
| | | GCACATTGCCAATCTGGCCGGCAGCCCCGCCATTAAGAAGGGCAT |
| | | CCTGCAGACAGTGAAGGTGGTGGACGAGCTCGTGAAAGTGATGG |
| | | GCCGGCACAAGCCCGAGAACATCGTGATCGAAATGGCCAGAGAG |
| | | AACCAGACCACCCAGAAGGGACAGAAGAACAGCCGCGAGAGAAT |
| | | GAAGCGGATCGAAGAGGGCATCAAAGAGCTGGGCAGCCAGATCC |
| | | TGAAAGAACACCCCGTGGAAAACACCCAGCTGCAGAACGAGAAG |
| | | CTGTACCTGTACTACCTGCAGAATGGGCGGGATATGTACGTGGAC |
| | | CAGGAACTGGACATCAACCGGCTGTCCGACTACGATGTGGACGCT |
| | | ATCGTGCCTCAGAGCTTTCTGAAGGACGACTCCATCGATAACAAAG |
| | | TGCTGACTCGGAGCGACAAGAACCGGGGCAAGAGCGACAACGTG |
| | | CCCTCCGAAGAGGTCGTGAAGAAGATGAAGAACTACTGGCGCCAG |
| | | CTGCTGAATGCCAAGCTGATTACCCAGAGGAAGTTCGACAATCTG |
| | | ACCAAGGCCGAGAGAGGCGGCCTGAGCGAACTGGATAAGGCCGG |
| | | CTTCATCAAGAGACAGCTGGTGGAAACCCGGCAGATCACAAAGCA |
| | | CGTGGCACAGATCCTGGACTCCCGGATGAACACTAAGTACGACGA |
| | | GAACGACAAACTGATCCGGGAAGTGAAAGTGATCACCCTGAAGTC |
| | | CAAGCTGGTGTCCGATTTCCGGAAGGATTTCCAGTTTTACAAAGTG |
| | | CGCGAGATCAACAACTACCACCACGCCCACGACGCCTACCTGAAC |
| | | GCCGTCGTGGGAACCGCCCTGATCAAAAAGTACCCTAAGCTGGAA |
| | | AGCGAGTTCGTGTACGGCGACTACAAGGTGTACGACGTGCGGAA |
| | | GATGATCGCCAAGAGCGAGCAGGAAATCGGCAAGGCTACCGCCA |
| | | AGTACTTCTTCTACAGCAACATCATGAACTTTTTCAAGACCGAGATT |
| | | ACCCTGGCCAACGGCGAGATCCGGAAGCGGCCTCTGATCGAGACA |
| | | AACGGCGAAACAGGCGAGATCGTGTGGGATAAGGGCCGGGACTT |
| | | TGCCACCGTGCGGAAAGTGCTGTCTATGCCCCAAGTGAATATCGT |
| | | GAAAAAGACCGAGGTGCAGACAGGCGGCTTCAGCAAAGAGTCTA |
| | | TCCTGCCCAAGAGGAACAGCGACAAGCTGATCGCCAGAAAGAAG |
| | | GACTGGGACCCTAAGAAGTACGGCGGCTTCGACAGCCCCACCGTG |
| | | GCCTATTCTGTGCTGGTGGTGGCCAAAGTGGAAAAGGGCAAGTCC |
| | | AAGAAACTGAAGAGTGTGAAAGAGCTGCTGGGGATCACCATCATG |
| | | GAAAGAAGCAGCTTCGAGAAGAATCCCATCGACTTTCTGGAAGCC |
| | | AAGGGCTACAAAGAAGTGAAAAAGGACCTGATCATCAAGCTGCCT |
| | | AAGTACTCCCTGTTCGAGCTGGAAAACGGCCGGAAGAGAATGCTG |
| | | GCCTCTGCCGGCGAACTGCAGAAGGGAAACGAACTGGCCCTGCCC |
| | | TCCAAATATGTGAACTTCCTGTACCTGGCCAGCCACTATGAGAAGC |
| | | TGAAGGGCTCCCCCGAGGATAATGAGCAGAAACAGCTGTTTGTGG |
| | | AACAGCACAAACACTACCTGGACGAGATCATCGAGCAGATCAGCG |
| | | AGTTCTCCAAGAGAGTGATCCTGGCCGACGCTAATCTGGACAAGG |
| | | TGCTGAGCGCCTACAACAAGCACAGAGACAAGCCTATCAGAGAGC |
| | | AGGCCGAGAATATCATCCACCTGTTTACCCTGACCAATCTGGGAGC |
| | | CCCTGCCGCCTTCAAGTACTTTGACACCACCATCGACCGGAAGAGG |
| | | TACACCAGCACCAAAGAGGTGCTGGACGCCACCCTGATCCACCAG |
| | | AGCATCACCGGCCTGTACGAGACACGGATCGACCTGTCTCAGCTG |
| | | GGAGGCGACAGCGGAAGTGAGACCCCAGGTACATCCGAATCAGC |
| | | AACGCCTGAAAGCACCGGTCGGACACTGGTGACCTTCAAGGATGT |
| | | ATTTGTGGACTTCACCAGGGAGGAGTGGAAGCTGCTGGACACTGC |
| | | TCAGCAGATCGTGTACAGAAATGTGATGCTGGAGAACTATAAGAA |
| | | CCTGGTTTCCTTGGGTTATCAGCTTACTAAGCCAGATGTGATCCTCC |
| | | GGTTGGAGAAGGGAGAAGAGCCCCCAAAAAAGAAGAGAAAGGT |
| | | ACCGAAGAAAAAAAGAAAGGTC |
| | Fusion Protein 2 | MNHDQEFDPPKVYPPVPAEKRKPIRVLSLFDGIATGLLVLKDLGIQVD |
| | Amino Acid | RYIASEVCEDSITVGMVRHQGKIMYVGDVRSVTQKHIQEWGPFDLVI |
| | Sequence | GGSPCNDLSIVNPARKGLYEGTGRLFFEFYRLLHDARPKEGDDRPFFW |
| | 3A-3L-NLS- | LFENVVAMGVSDKRDISRFLESNPVMIDAKEVSAAHRARYFWGNLP |
| | dCas9-NLS-KRAB | GMNRPLASTVNDKLELQECLEHGRIAKFSKVRTITTRSNSIKQGKDQH |
| | | FPVFMNEKEDILWCTEMERVFGFPVHYTDVSNMSRLARQRLLGRSW |
| | | SVPVIRHLFAPLKEYFACVSSGNSNANSRGPSFSSGLVPLSLRGSHNPL |
| | | EMFETVPVWRRQPVRVLSLFEDIKKELTSLGFLESGSDPGQLKHVVDV |
| | | TDTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSLGGPSSGAPPPSGGSPAGSPTSTEEGTSES |
| | | ATPESGPGTSTEPSEGSAPGSPAGSPTSTEEGTSTEPSEGSAPGTSTEP |
| | | SEPKKKRKVMDKKYSIGLAIGTNSVGWAVITDEYKVPSKKFKVLGNTD |
| | | RHSIKKNLIGALLFDSGETAEATRLKRTARRRYTRRKNRICYLQEIFSNE |
| | | MAKVDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKLFIQ |
| | | LVQTYNQLFEENPINASGVDAKAILSARLSKSRRLENLIAQLPGEKKNG |
| | | LFGNLIALSLGLTPNFKSNFDLAEDAKLQLSKDTYDDDLDNLLAQIGDQ |
| | | YADLFLAAKNLSDAILLSDILRVNTEITKAPLSASMIKRYDEHHQDLTLL |
| | | KALVRQQLPEKYKEIFFDQSKNGYAGYIDGGASQEEFYKFIKPILEKMD |
| | | GTEELLVKLNREDLLRKQRTFDNGSIPHQIHLGELHAILRRQEDFYPFLK |
| | | DNREKIEKILTFRIPYYVGPLARGNSRFAWMTRKSEETITPWNFEEVV |
| | | DKGASAQSFIERMTNFDKNLPNEKVLPKHSLLYEYFTVYNELTKVKYVT |
| | | EGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDRE |
| | | MIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQK |
| | | GQKNSRERMKRIEEGIKELGSQILKEHPVENTQLQNEKLYLYYLQNGR |
| | | DMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKA |
| | | GFIKRQLVETRQITKHVAQILDSRMNTKYDENDKLIREVKVITLKSKLVS |
| | | DFRKDFQFYKVREINNYHHAHDAYLNAVVGTALIKKYPKLESEFVYGD |
| | | YKVYDVRKMIAKSEQEIGKATAKYFFYSNIMNFFKTEITLANGEIRKRPL |
| | | IETNGETGEIVWDKGRDFATVRKVLSMPQVNIVKKTEVQTGGFSKESI |
| | | LPKRNSDKLIARKKDWDPKKYGGFDSPTVAYSVLVVAKVEKGKSKKLK |
| | | SVKELLGITIMERSSFEKNPIDFLEAKGYKEVKKDLIIKLPKYSLFELENGR |
| | | KRMLASAGELQKGNELALPSKYVNFLYLASHYEKLKGSPEDNEQKQLF |
| | | VEQHKHYLDEIIEQISEFSKRVILADANLDKVLSAYNKHRDKPIREQAE |
| | | NIIHLFTLTNLGAPAAFKYFDTTIDRKRYTSTKEVLDATLIHQSITGLYET |
| | | RIDLSQLGGDPKKKRKVSGSETPGTSESATPESTGRTLVTFKDVFVDFT |
| | | REEWKLLDTAQQIVYRNVMLENYKNLVSLGYQLTKPDVILRLEKGEEP |
| | Fusion Protein 2 | ATGAACCACGACCAGGAATTTGACCCTCCAAAGGTTTACCCACCTG |
| | DNA Sequence | TCCCAGCTGAGAAGAGGAAGCCCATCCGGGTGCTGTCTCTCTTTGA |
| | | TGGAATCGCTACAGGGCTCCTGGTGCTGAAGGACTTGGGCATTCA |
| | | GGTGGACCGCTACATTGCCTCGGAGGTGTGTGAGGACTCCATCAC |
| | | GGTGGGCATGGTGCGGCACCAGGGGAAGATCATGTACGTCGGGG |
| | | ACGTCCGCAGCGTCACACAGAAGCATATCCAGGAGTGGGGCCCAT |
| | | TCGATCTGGTGATTGGGGGCAGTCCCTGCAATGACCTCTCCATCGT |
| | | CAACCCTGCTCGCAAGGGCCTCTACGAGGGCACTGGCCGGCTCTT |
| | | CTTTGAGTTCTACCGCCTCCTGCATGATGCGCGGCCCAAGGAGGG |
| | | AGATGATCGCCCCTTCTTCTGGCTCTTTGAGAATGTGGTGGCCATG |
| | | GGCGTTAGTGACAAGAGGGACATCTCGCGATTTCTCGAGTCCAAC |
| | | CCTGTGATGATTGATGCCAAAGAAGTGTCAGCTGCACACAGGGCC |
| | | CGCTACTTCTGGGGTAACCTTCCCGGTATGAACAGGCCGTTGGCAT |
| | | CCACTGTGAATGATAAGCTGGAGCTGCAGGAGTGTCTGGAGCATG |
| | | GCAGGATAGCCAAGTTCAGCAAAGTGAGGACCATTACTACGAGGT |
| | | CAAACTCCATAAAGCAGGGCAAAGACCAGCATTTTCCTGTCTTCAT |
| | | GAATGAGAAAGAGGACATCTTATGGTGCACTGAAATGGAAAGGG |
| | | TATTTGGTTTCCCAGTCCACTATACTGACGTCTCCAACATGAGCCGC |
| | | TTGGCGAGGCAGAGACTGCTGGGCCGGTCATGGAGCGTGCCAGT |
| | | CATCCGCCACCTCTTCGCTCCGCTGAAGGAGTATTTTGCGTGTGTG |
| | | TCTAGCGGCAATAGTAACGCTAACAGCCGCGGGCCGAGCTTCAGC |
| | | AGCGGCCTGGTGCCGTTAAGCTTGCGCGGCAGCCATAATCCCCTT |
| | | GAGATGTTCGAAACCGTGCCTGTGTGGAGGAGACAGCCAGTCCG |
| | | GGTGCTGTCCCTTTTTGAAGACATCAAGAAAGAGCTGACGAGTTT |
| | | GGGCTTTTTGGAAAGTGGTTCTGACCCGGGACAACTGAAGCATGT |
| | | GGTTGATGTCACAGACACAGTGAGGAAGGATGTGGAGGAGTGGG |
| | | GACCCTTCGATCTTGTGTACGGCGCCACACCTCCCCTGGGCCACAC |
| | | CTGTGACCGTCCTCCCAGCTGGTACCTGTTCCAGTTCCACCGGCTCC |
| | | TGCAGTACGCACGGCCCAAGCCAGGCAGCCCCAGGCCCTTCTTCT |
| | | GGATGTTCGTGGACAATCTGGTGCTGAACAAGGAAGACCTGGACG |
| | | TCGCATCTCGCTTCCTGGAGATGGAGCCAGTCACCATCCCAGATGT |
| | | CCACGGCGGATCCTTGCAGAATGCTGTCCGCGTGTGGAGCAACAT |
| | | CCCAGCCATAAGGAGCAGGCACTGGGCTCTGGTTTCGGAAGAAGA |
| | | ATTGTCCCTGCTGGCCCAGAACAAGCAGAGCTCGAAGCTCGCGGC |
| | | CAAGTGGCCCACCAAGCTGGTGAAGAACTGCTTTCTCCCCCTAAGA |
| | | GAATATTTCAAGTATTTTTCAACAGAACTCACTTCCTCTTTAGGAGG |
| | | GCCGAGCTCTGGCGCACCCCCACCAAGTGGAGGGTCTCCTGCCGG |
| | | GTCCCCAACATCTACTGAAGAAGGCACCAGCGAATCCGCAACGCC |
| | | CGAGTCAGGCCCTGGTACCTCCACAGAACCATCTGAAGGTAGTGC |
| | | GCCTGGTTCCCCAGCTGGAAGCCCTACTTCCACCGAAGAAGGCAC |
| | | GTCAACCGAACCAAGTGAAGGATCTGCCCCTGGGACCAGCACTGA |
| | | ACCATCTGAGCCAAAAAAGAAGAGAAAGGTAATGGACAAGAAGT |
| | | ACAGCATCGGCCTGGCCATCGGCACCAACTCTGTGGGCTGGGCCG |
| | | TGATCACCGACGAGTACAAGGTGCCCAGCAAGAAATTCAAGGTGC |
| | | TGGGCAACACCGACCGGCACAGCATCAAGAAGAACCTGATCGGCG |
| | | CCCTGCTGTTCGACAGCGGAGAAACAGCCGAGGCCACCCGGCTGA |
| | | AGAGAACCGCCAGAAGAAGATACACCAGACGGAAGAACCGGATC |
| | | TGCTATCTGCAAGAGATCTTCAGCAACGAGATGGCCAAGGTGGAC |
| | | GACAGCTTCTTCCACAGACTGGAAGAGTCCTTCCTGGTGGAAGAG |
| | | GATAAGAAGCACGAGCGGCACCCCATCTTCGGCAACATCGTGGAC |
| | | GAGGTGGCCTACCACGAGAAGTACCCCACCATCTACCACCTGAGA |
| | | AAGAAACTGGTGGACAGCACCGACAAGGCCGACCTGCGGCTGATC |
| | | TATCTGGCCCTGGCCCACATGATCAAGTTCCGGGGCCACTTCCTGA |
| | | TCGAGGGCGACCTGAACCCCGACAACAGCGACGTGGACAAGCTGT |
| | | TCATCCAGCTGGTGCAGACCTACAACCAGCTGTTCGAGGAAAACC |
| | | CCATCAACGCCAGCGGCGTGGACGCCAAGGCCATCCTGTCTGCCA |
| | | GACTGAGCAAGAGCAGACGGCTGGAAAATCTGATCGCCCAGCTGC |
| | | CCGGCGAGAAGAAGAATGGCCTGTTCGGCAACCTGATTGCCCTGA |
| | | GCCTGGGCCTGACCCCCAACTTCAAGAGCAACTTCGACCTGGCCG |
| | | AGGATGCCAAACTGCAGCTGAGCAAGGACACCTACGACGACGACC |
| | | TGGACAACCTGCTGGCCCAGATCGGCGACCAGTACGCCGACCTGT |
| | | TTCTGGCCGCCAAGAACCTGTCCGACGCCATCCTGCTGAGCGACAT |
| | | CCTGAGAGTGAACACCGAGATCACCAAGGCCCCCCTGAGCGCCTC |
| | | TATGATCAAGAGATACGACGAGCACCACCAGGACCTGACCCTGCT |
| | | GAAAGCTCTCGTGCGGCAGCAGCTGCCTGAGAAGTACAAAGAGAT |
| | | TTTCTTCGACCAGAGCAAGAACGGCTACGCCGGCTACATCGATGG |
| | | CGGAGCCAGCCAGGAAGAGTTCTACAAGTTCATCAAGCCCATCCT |
| | | GGAAAAGATGGACGGCACCGAGGAACTGCTCGTGAAGCTGAACA |
| | | GAGAGGACCTGCTGCGGAAGCAGCGGACCTTCGACAACGGCAGC |
| | | ATCCCCCACCAGATCCACCTGGGAGAGCTGCACGCCATTCTGCGGC |
| | | GGCAGGAAGATTTTTACCCATTCCTGAAGGACAACCGGGAAAAGA |
| | | TCGAGAAGATCCTGACCTTCCGCATCCCCTACTACGTGGGCCCTCT |
| | | GGCCAGGGGAAACAGCAGATTCGCCTGGATGACCAGAAAGAGCG |
| | | AGGAAACCATCACCCCCTGGAACTTCGAGGAAGTGGTGGACAAGG |
| | | GCGCCAGCGCCCAGAGCTTCATCGAGCGGATGACCAACTTCGATA |
| | | AGAACCTGCCCAACGAGAAGGTGCTGCCCAAGCACAGCCTGCTGT |
| | | ACGAGTACTTCACCGTGTACAACGAGCTGACCAAAGTGAAATACG |
| | | TGACCGAGGGAATGAGAAAGCCCGCCTTCCTGAGCGGCGAGCAG |
| | | AAAAAAGCCATCGTGGACCTGCTGTTCAAGACCAACCGGAAAGTG |
| | | ACCGTGAAGCAGCTGAAAGAGGACTACTTCAAGAAAATCGAGTGC |
| | | TTCGACTCCGTGGAAATCTCCGGCGTGGAAGATCGGTTCAACGCCT |
| | | CCCTGGGCACATACCACGATCTGCTGAAAATTATCAAGGACAAGG |
| | | ACTTCCTGGACAATGAGGAAAACGAGGACATTCTGGAAGATATCG |
| | | TGCTGACCCTGACACTGTTTGAGGACAGAGAGATGATCGAGGAAC |
| | | GGCTGAAAACCTATGCCCACCTGTTCGACGACAAAGTGATGAAGC |
| | | AGCTGAAGCGGCGGAGATACACCGGCTGGGGCAGGCTGAGCCGG |
| | | AAGCTGATCAACGGCATCCGGGACAAGCAGTCCGGCAAGACAATC |
| | | CTGGATTTCCTGAAGTCCGACGGCTTCGCCAACAGAAACTTCATGC |
| | | AGCTGATCCACGACGACAGCCTGACCTTTAAAGAGGACATCCAGA |
| | | AAGCCCAGGTGTCCGGCCAGGGCGATAGCCTGCACGAGCACATTG |
| | | CCAATCTGGCCGGCAGCCCCGCCATTAAGAAGGGCATCCTGCAGA |
| | | CAGTGAAGGTGGTGGACGAGCTCGTGAAAGTGATGGGCCGGCAC |
| | | AAGCCCGAGAACATCGTGATCGAAATGGCCAGAGAGAACCAGAC |
| | | CACCCAGAAGGGACAGAAGAACAGCCGCGAGAGAATGAAGCGGA |
| | | TCGAAGAGGGCATCAAAGAGCTGGGCAGCCAGATCCTGAAAGAA |
| | | CACCCCGTGGAAAACACCCAGCTGCAGAACGAGAAGCTGTACCTG |
| | | TACTACCTGCAGAATGGGCGGGATATGTACGTGGACCAGGAACTG |
| | | GACATCAACCGGCTGTCCGACTACGATGTGGACGCTATCGTGCCTC |
| | | AGAGCTTTCTGAAGGACGACTCCATCGATAACAAAGTGCTGACTC |
| | | GGAGCGACAAGAACCGGGGCAAGAGCGACAACGTGCCCTCCGAA |
| | | GAGGTCGTGAAGAAGATGAAGAACTACTGGCGCCAGCTGCTGAA |
| | | TGCCAAGCTGATTACCCAGAGGAAGTTCGACAATCTGACCAAGGC |
| | | CGAGAGAGGCGGCCTGAGCGAACTGGATAAGGCCGGCTTCATCA |
| | | AGAGACAGCTGGTGGAAACCCGGCAGATCACAAAGCACGTGGCA |
| | | CAGATCCTGGACTCCCGGATGAACACTAAGTACGACGAGAACGAC |
| | | AAACTGATCCGGGAAGTGAAAGTGATCACCCTGAAGTCCAAGCTG |
| | | GTGTCCGATTTCCGGAAGGATTTCCAGTTTTACAAAGTGCGCGAGA |
| | | TCAACAACTACCACCACGCCCACGACGCCTACCTGAACGCCGTCGT |
| | | GGGAACCGCCCTGATCAAAAAGTACCCTAAGCTGGAAAGCGAGTT |
| | | CGTGTACGGCGACTACAAGGTGTACGACGTGCGGAAGATGATCGC |
| | | CAAGAGCGAGCAGGAAATCGGCAAGGCTACCGCCAAGTACTTCTT |
| | | CTACAGCAACATCATGAACTTTTTCAAGACCGAGATTACCCTGGCC |
| | | AACGGCGAGATCCGGAAGCGGCCTCTGATCGAGACAAACGGCGA |
| | | AACAGGCGAGATCGTGTGGGATAAGGGCCGGGACTTTGCCACCG |
| | | TGCGGAAAGTGCTGTCTATGCCCCAAGTGAATATCGTGAAAAAGA |
| | | CCGAGGTGCAGACAGGCGGCTTCAGCAAAGAGTCTATCCTGCCCA |
| | | AGAGGAACAGCGACAAGCTGATCGCCAGAAAGAAGGACTGGGAC |
| | | CCTAAGAAGTACGGCGGCTTCGACAGCCCCACCGTGGCCTATTCTG |
| | | TGCTGGTGGTGGCCAAAGTGGAAAAGGGCAAGTCCAAGAAACTG |
| | | AAGAGTGTGAAAGAGCTGCTGGGGATCACCATCATGGAAAGAAG |
| | | CAGCTTCGAGAAGAATCCCATCGACTTTCTGGAAGCCAAGGGCTA |
| | | CAAAGAAGTGAAAAAGGACCTGATCATCAAGCTGCCTAAGTACTC |
| | | CCTGTTCGAGCTGGAAAACGGCCGGAAGAGAATGCTGGCCTCTGC |
| | | CGGCGAACTGCAGAAGGGAAACGAACTGGCCCTGCCCTCCAAATA |
| | | TGTGAACTTCCTGTACCTGGCCAGCCACTATGAGAAGCTGAAGGG |
| | | CTCCCCCGAGGATAATGAGCAGAAACAGCTGTTTGTGGAACAGCA |
| | | CAAACACTACCTGGACGAGATCATCGAGCAGATCAGCGAGTTCTC |
| | | CAAGAGAGTGATCCTGGCCGACGCTAATCTGGACAAGGTGCTGAG |
| | | CGCCTACAACAAGCACAGAGACAAGCCTATCAGAGAGCAGGCCGA |
| | | GAATATCATCCACCTGTTTACCCTGACCAATCTGGGAGCCCCTGCC |
| | | GCCTTCAAGTACTTTGACACCACCATCGACCGGAAGAGGTACACCA |
| | | GCACCAAAGAGGTGCTGGACGCCACCCTGATCCACCAGAGCATCA |
| | | CCGGCCTGTACGAGACACGGATCGACCTGTCTCAGCTGGGAGGCG |
| | | ACCCAAAAAAGAAGAGAAAGGTAAGCGGAAGTGAGACCCCAGGT |
| | | ACATCCGAATCAGCAACGCCTGAAAGCACCGGTCGGACACTGGTG |
| | | ACCTTCAAGGATGTATTTGTGGACTTCACCAGGGAGGAGTGGAAG |
| | | CTGCTGGACACTGCTCAGCAGATCGTGTACAGAAATGTGATGCTG |
| | | GAGAACTATAAGAACCTGGTTTCCTTGGGTTATCAGCTTACTAAGC |
| | | CAGATGTGATCCTCCGGTTGGAGAAGGGAGAAGAGCCC |
| | Fusion Protein 3 | MNHDQEFDPPKVYPPVPAEKRKPIRVLSLFDGIATGLLVLKDLGIQVD |
| | Amino Acid | RYIASEVCEDSITVGMVRHQGKIMYVGDVRSVTQKHIQEWGPFDLVI |
| | Sequence | GGSPCNDLSIVNPARKGLYEGTGRLFFEFYRLLHDARPKEGDDRPFFW |
| | 3A-ADD-hm 3L- | LFENVVAMGVSDKRDISRFLESNPVMIDAKEVSAAHRARYFWGNLP |
| | NLS-dCas9-NLS- | GMNRPLASTVNDKLELQECLEHGRIAKFSKVRTITTRSNSIKQGKDQH |
| | KRAB | FPVFMNEKEDILWCTEMERVFGFPVHYTDVSNMSRLARQRLLGRSW |
| | | SVPVIRHLFAPLKEYFACVSSGNSNANSRGPSFSSGLVPLSLRGSHMEV |
| | | KVNRRSIEDICLCCGTLQVYTRHPLFEGGLCAPCKDKFLESLFLYDDDG |
| | | HQSYCTICCSGGTLFICESPDCTRCYCFECVDILVGPGTSERINAMACW |
| | | VCFLCLPFSRSGLLQRRKRWRHQLKAFHDQEGAGPMEIYKTVSAWK |
| | | RQPVRVLSLFRNIDKVLKSLGFLESGSGSGGGTLKYVEDVTNVVRRDV |
| | | EKWGPFDLVYGSTQPLGSSCDRCPGWYMFQFHRILQYALPRQESQR |
| | | PFFWIFMDNLLLTEDDQETTTRFLQTEAVTLQDVRGRDYQNAMRV |
| | | WSNIPGLKSKHAPLTPKEEEYLQAQVRSRSKLDAPKVDLLVKNCLLPLR |
| | | EYFKYFSQNSLPLGGPSSGAPPPSGGSPAGSPTSTEEGTSESATPESGP |
| | | GTSTEPSEGSAPGSPAGSPTSTEEGTSTEPSEGSAPGTSTEPSEPKKKR |
| | | KVMDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDP |
| | | KKKRKVSGSETPGTSESATPESTGRTLVTFKDVFVDFTREEWKLLDTA |
| | | QQIVYRNVMLENYKNLVSLGYQLTKPDVILRLEKGEEP |
| | Fusion Protein 3 | ATGAACCATGACCAGGAATTTGACCCCCCAAAGGTTTACCCACCTG |
| | DNA Sequence | TGCCAGCTGAGAAGAGGAAGCCCATCCGCGTGCTGTCTCTCTTTGA |
| | | TGGGATTGCTACAGGGCTCCTGGTGCTGAAGGACCTGGGCATCCA |
| | | AGTGGACCGCTACATTGCCTCCGAGGTGTGTGAGGACTCCATCAC |
| | | GGTGGGCATGGTGCGGCACCAGGGAAAGATCATGTACGTCGGGG |
| | | ACGTCCGCAGCGTCACACAGAAGCATATCCAGGAGTGGGGCCCAT |
| | | TCGACCTGGTGATTGGAGGCAGTCCCTGCAATGACCTCTCCATTGT |
| | | CAACCCTGCCCGCAAGGGACTTTATGAGGGTACTGGCCGCCTCTTC |
| | | TTTGAGTTCTACCGCCTCCTGCATGATGCGCGGCCCAAGGAGGGA |
| | | GATGATCGCCCCTTCTTCTGGCTCTTTGAGAATGTGGTGGCCATGG |
| | | GCGTTAGTGACAAGAGGGACATCTCGCGATTTCTTGAGTCTAACCC |
| | | CGTGATGATTGACGCCAAAGAAGTGTCTGCTGCACACAGGGCCCG |
| | | TTACTTCTGGGGTAACCTTCCTGGCATGAACAGGCCTTTGGCATCC |
| | | ACTGTGAATGATAAGCTGGAGCTGCAAGAGTGTCTGGAGCACGGC |
| | | AGAATAGCCAAGTTCAGCAAAGTGAGGACCATTACCACCAGGTCA |
| | | AACTCTATAAAGCAGGGCAAAGACCAGCATTTCCCCGTCTTCATGA |
| | | ACGAGAAGGAGGACATCCTGTGGTGCACTGAAATGGAAAGGGTG |
| | | TTTGGCTTCCCCGTCCACTACACAGACGTCTCCAACATGAGCCGCTT |
| | | GGCGAGGCAGAGACTGCTGGGCCGATCGTGGAGCGTGCCGGTCA |
| | | TCCGCCACCTCTTCGCTCCGCTGAAGGAATATTTTGCTTGTGTGTCT |
| | | AGCGGCAATAGTAACGCTAACAGCCGCGGGCCGAGCTTCAGCAGC |
| | | GGCCTGGTGCCGTTAAGCTTGCGCGGCAGCCATATGGAAGTCAAA |
| | | GTGAACCGACGGAGCATTGAAGACATCTGCCTCTGCTGTGGAACT |
| | | CTCCAGGTGTACACTCGGCACCCCTTGTTTGAGGGAGGGTTATGTG |
| | | CCCCATGTAAGGATAAGTTCCTGGAGTCCCTCTTCCTGTATGATGA |
| | | TGATGGACACCAGAGTTACTGCACCATCTGCTGTTCCGGGGGTACC |
| | | CTGTTCATCTGTGAGAGCCCCGACTGTACCAGATGCTACTGTTTCG |
| | | AGTGTGTGGACATCCTGGTGGGCCCCGGGACCTCAGAGAGGATCA |
| | | ATGCCATGGCCTGCTGGGTTTGCTTCCTGTGCCTGCCCTTCTCACG |
| | | GAGTGGACTGCTGCAGAGGCGCAAGAGGTGGCGGCACCAGCTGA |
| | | AGGCCTTCCATGATCAAGAGGGAGCGGGCCCTATGGAGATATACA |
| | | AGACAGTGTCTGCATGGAAGAGACAGCCAGTGCGGGTACTGAGC |
| | | CTCTTCAGAAACATCGACAAGGTACTAAAGAGTTTGGGCTTCTTGG |
| | | AAAGCGGTTCTGGTTCTGGGGGAGGAACGCTGAAGTACGTGGAA |
| | | GATGTCACAAATGTCGTGAGGAGAGACGTGGAGAAATGGGGCCC |
| | | CTTTGACCTGGTGTACGGCTCGACGCAGCCCCTAGGCAGCTCTTGT |
| | | GATCGCTGTCCCGGCTGGTACATGTTCCAGTTCCACCGGATCCTGC |
| | | AGTATGCGCTGCCTCGCCAGGAGAGTCAGCGGCCCTTCTTCTGGAT |
| | | ATTCATGGACAATCTGCTGCTGACTGAGGATGACCAAGAGACAAC |
| | | TACCCGCTTCCTTCAGACAGAGGCTGTGACCCTCCAGGATGTCCGT |
| | | GGCAGAGACTACCAGAATGCTATGCGGGTGTGGAGCAACATTCCA |
| | | GGGCTGAAGAGCAAGCATGCGCCCCTGACCCCAAAGGAAGAAGA |
| | | GTATCTGCAAGCCCAAGTCAGAAGCAGGAGCAAGCTGGACGCCCC |
| | | GAAAGTTGACCTCCTGGTGAAGAACTGCCTTCTCCCGCTGAGAGA |
| | | GTACTTCAAGTATTTTTCTCAAAACTCACTTCCTCTTGGAGGGCCGA |
| | | GCTCTGGCGCACCCCCACCAAGTGGAGGGTCTCCTGCCGGGTCCC |
| | | CAACATCTACTGAAGAAGGCACCAGCGAATCCGCAACGCCCGAGT |
| | | CAGGCCCTGGTACCTCCACAGAACCATCTGAAGGTAGTGCGCCTG |
| | | GTTCCCCAGCTGGAAGCCCTACTTCCACCGAAGAAGGCACGTCAA |
| | | CCGAACCAAGTGAAGGATCTGCCCCTGGGACCAGCACTGAACCAT |
| | | CTGAGCCAAAAAAGAAGAGAAAGGTAATGGACAAGAAGTACAGC |
| | | ATCGGCCTGGCCATCGGCACCAACTCTGTGGGCTGGGCCGTGATC |
| | | ACCGACGAGTACAAGGTGCCCAGCAAGAAATTCAAGGTGCTGGGC |
| | | AACACCGACCGGCACAGCATCAAGAAGAACCTGATCGGCGCCCTG |
| | | CTGTTCGACAGCGGAGAAACAGCCGAGGCCACCCGGCTGAAGAG |
| | | AACCGCCAGAAGAAGATACACCAGACGGAAGAACCGGATCTGCTA |
| | | TCTGCAAGAGATCTTCAGCAACGAGATGGCCAAGGTGGACGACAG |
| | | CTTCTTCCACAGACTGGAAGAGTCCTTCCTGGTGGAAGAGGATAA |
| | | GAAGCACGAGCGGCACCCCATCTTCGGCAACATCGTGGACGAGGT |
| | | GGCCTACCACGAGAAGTACCCCACCATCTACCACCTGAGAAAGAA |
| | | ACTGGTGGACAGCACCGACAAGGCCGACCTGCGGCTGATCTATCT |
| | | GGCCCTGGCCCACATGATCAAGTTCCGGGGCCACTTCCTGATCGA |
| | | GGGCGACCTGAACCCCGACAACAGCGACGTGGACAAGCTGTTCAT |
| | | CCAGCTGGTGCAGACCTACAACCAGCTGTTCGAGGAAAACCCCAT |
| | | CAACGCCAGCGGCGTGGACGCCAAGGCCATCCTGTCTGCCAGACT |
| | | GAGCAAGAGCAGACGGCTGGAAAATCTGATCGCCCAGCTGCCCG |
| | | GCGAGAAGAAGAATGGCCTGTTCGGCAACCTGATTGCCCTGAGCC |
| | | TGGGCCTGACCCCCAACTTCAAGAGCAACTTCGACCTGGCCGAGG |
| | | ATGCCAAACTGCAGCTGAGCAAGGACACCTACGACGACGACCTGG |
| | | ACAACCTGCTGGCCCAGATCGGCGACCAGTACGCCGACCTGTTTCT |
| | | GGCCGCCAAGAACCTGTCCGACGCCATCCTGCTGAGCGACATCCT |
| | | GAGAGTGAACACCGAGATCACCAAGGCCCCCCTGAGCGCCTCTAT |
| | | GATCAAGAGATACGACGAGCACCACCAGGACCTGACCCTGCTGAA |
| | | AGCTCTCGTGCGGCAGCAGCTGCCTGAGAAGTACAAAGAGATTTT |
| | | CTTCGACCAGAGCAAGAACGGCTACGCCGGCTACATCGATGGCGG |
| | | AGCCAGCCAGGAAGAGTTCTACAAGTTCATCAAGCCCATCCTGGA |
| | | AAAGATGGACGGCACCGAGGAACTGCTCGTGAAGCTGAACAGAG |
| | | AGGACCTGCTGCGGAAGCAGCGGACCTTCGACAACGGCAGCATCC |
| | | CCCACCAGATCCACCTGGGAGAGCTGCACGCCATTCTGCGGCGGC |
| | | AGGAAGATTTTTACCCATTCCTGAAGGACAACCGGGAAAAGATCG |
| | | AGAAGATCCTGACCTTCCGCATCCCCTACTACGTGGGCCCTCTGGC |
| | | CAGGGGAAACAGCAGATTCGCCTGGATGACCAGAAAGAGCGAGG |
| | | AAACCATCACCCCCTGGAACTTCGAGGAAGTGGTGGACAAGGGCG |
| | | CCAGCGCCCAGAGCTTCATCGAGCGGATGACCAACTTCGATAAGA |
| | | ACCTGCCCAACGAGAAGGTGCTGCCCAAGCACAGCCTGCTGTACG |
| | | AGTACTTCACCGTGTACAACGAGCTGACCAAAGTGAAATACGTGA |
| | | CCGAGGGAATGAGAAAGCCCGCCTTCCTGAGCGGCGAGCAGAAA |
| | | AAAGCCATCGTGGACCTGCTGTTCAAGACCAACCGGAAAGTGACC |
| | | GTGAAGCAGCTGAAAGAGGACTACTTCAAGAAAATCGAGTGCTTC |
| | | GACTCCGTGGAAATCTCCGGCGTGGAAGATCGGTTCAACGCCTCC |
| | | CTGGGCACATACCACGATCTGCTGAAAATTATCAAGGACAAGGAC |
| | | TTCCTGGACAATGAGGAAAACGAGGACATTCTGGAAGATATCGTG |
| | | CTGACCCTGACACTGTTTGAGGACAGAGAGATGATCGAGGAACGG |
| | | CTGAAAACCTATGCCCACCTGTTCGACGACAAAGTGATGAAGCAG |
| | | CTGAAGCGGCGGAGATACACCGGCTGGGGCAGGCTGAGCCGGAA |
| | | GCTGATCAACGGCATCCGGGACAAGCAGTCCGGCAAGACAATCCT |
| | | GGATTTCCTGAAGTCCGACGGCTTCGCCAACAGAAACTTCATGCAG |
| | | CTGATCCACGACGACAGCCTGACCTTTAAAGAGGACATCCAGAAA |
| | | GCCCAGGTGTCCGGCCAGGGCGATAGCCTGCACGAGCACATTGCC |
| | | AATCTGGCCGGCAGCCCCGCCATTAAGAAGGGCATCCTGCAGACA |
| | | GTGAAGGTGGTGGACGAGCTCGTGAAAGTGATGGGCCGGCACAA |
| | | GCCCGAGAACATCGTGATCGAAATGGCCAGAGAGAACCAGACCAC |
| | | CCAGAAGGGACAGAAGAACAGCCGCGAGAGAATGAAGCGGATCG |
| | | AAGAGGGCATCAAAGAGCTGGGCAGCCAGATCCTGAAAGAACAC |
| | | CCCGTGGAAAACACCCAGCTGCAGAACGAGAAGCTGTACCTGTAC |
| | | TACCTGCAGAATGGGGGGATATGTACGTGGACCAGGAACTGGA |
| | | CATCAACCGGCTGTCCGACTACGATGTGGACGCTATCGTGCCTCAG |
| | | AGCTTTCTGAAGGACGACTCCATCGATAACAAAGTGCTGACTCGG |
| | | AGCGACAAGAACCGGGGCAAGAGCGACAACGTGCCCTCCGAAGA |
| | | GGTCGTGAAGAAGATGAAGAACTACTGGCGCCAGCTGCTGAATGC |
| | | CAAGCTGATTACCCAGAGGAAGTTCGACAATCTGACCAAGGCCGA |
| | | GAGAGGCGGCCTGAGCGAACTGGATAAGGCCGGCTTCATCAAGA |
| | | GACAGCTGGTGGAAACCCGGCAGATCACAAAGCACGTGGCACAG |
| | | ATCCTGGACTCCCGGATGAACACTAAGTACGACGAGAACGACAAA |
| | | CTGATCCGGGAAGTGAAAGTGATCACCCTGAAGTCCAAGCTGGTG |
| | | TCCGATTTCCGGAAGGATTTCCAGTTTTACAAAGTGCGCGAGATCA |
| | | ACAACTACCACCACGCCCACGACGCCTACCTGAACGCCGTCGTGG |
| | | GAACCGCCCTGATCAAAAAGTACCCTAAGCTGGAAAGCGAGTTCG |
| | | TGTACGGCGACTACAAGGTGTACGACGTGCGGAAGATGATCGCCA |
| | | AGAGCGAGCAGGAAATCGGCAAGGCTACCGCCAAGTACTTCTTCT |
| | | ACAGCAACATCATGAACTTTTTCAAGACCGAGATTACCCTGGCCAA |
| | | CGGCGAGATCCGGAAGCGGCCTCTGATCGAGACAAACGGCGAAA |
| | | CAGGCGAGATCGTGTGGGATAAGGGCCGGGACTTTGCCACCGTG |
| | | CGGAAAGTGCTGTCTATGCCCCAAGTGAATATCGTGAAAAAGACC |
| | | GAGGTGCAGACAGGCGGCTTCAGCAAAGAGTCTATCCTGCCCAAG |
| | | AGGAACAGCGACAAGCTGATCGCCAGAAAGAAGGACTGGGACCC |
| | | TAAGAAGTACGGCGGCTTCGACAGCCCCACCGTGGCCTATTCTGT |
| | | GCTGGTGGTGGCCAAAGTGGAAAAGGGCAAGTCCAAGAAACTGA |
| | | AGAGTGTGAAAGAGCTGCTGGGGATCACCATCATGGAAAGAAGC |
| | | AGCTTCGAGAAGAATCCCATCGACTTTCTGGAAGCCAAGGGCTAC |
| | | AAAGAAGTGAAAAAGGACCTGATCATCAAGCTGCCTAAGTACTCC |
| | | CTGTTCGAGCTGGAAAACGGCCGGAAGAGAATGCTGGCCTCTGCC |
| | | GGCGAACTGCAGAAGGGAAACGAACTGGCCCTGCCCTCCAAATAT |
| | | GTGAACTTCCTGTACCTGGCCAGCCACTATGAGAAGCTGAAGGGC |
| | | TCCCCCGAGGATAATGAGCAGAAACAGCTGTTTGTGGAACAGCAC |
| | | AAACACTACCTGGACGAGATCATCGAGCAGATCAGCGAGTTCTCC |
| | | AAGAGAGTGATCCTGGCCGACGCTAATCTGGACAAGGTGCTGAGC |
| | | GCCTACAACAAGCACAGAGACAAGCCTATCAGAGAGCAGGCCGA |
| | | GAATATCATCCACCTGTTTACCCTGACCAATCTGGGAGCCCCTGCC |
| | | GCCTTCAAGTACTTTGACACCACCATCGACCGGAAGAGGTACACCA |
| | | GCACCAAAGAGGTGCTGGACGCCACCCTGATCCACCAGAGCATCA |
| | | CCGGCCTGTACGAGACACGGATCGACCTGTCTCAGCTGGGAGGCG |
| | | ACCCAAAAAAGAAGAGAAAGGTAAGCGGAAGTGAGACCCCAGGT |
| | | ACATCCGAATCAGCAACGCCTGAAAGCACCGGTCGGACACTGGTG |
| | | ACCTTCAAGGATGTATTTGTGGACTTCACCAGGGAGGAGTGGAAG |
| | | CTGCTGGACACTGCTCAGCAGATCGTGTACAGAAATGTGATGCTG |
| | | GAGAACTATAAGAACCTGGTTTCCTTGGGTTATCAGCTTACTAAGC |
| | | CAGATGTGATCCTCCGGTTGGAGAAGGGAGAAGAGCCC |
| | Fusion Protein 4 | MNHDQEFDPPKVYPPVPAEKRKPIRVLSLFDGIATGLLVLKDLGIQVD |
| | Amino Acid | RYIASEVCEDSITVGMVRHQGKIMYVGDVRSVTQKHIQEWGPFDLVI |
| | Sequence | GGSPCNDLSIVNPARKGLYEGTGRLFFEFYRLLHDARPKEGDDRPFFW |
| | 3A-ADD-h3L- | LFENVVAMGVSDKRDISRFLESNPVMIDAKEVSAAHRARYFWGNLP |
| | NLS-dCas9-NLS- | GMNRPLASTVNDKLELQECLEHGRIAKFSKVRTITTRSNSIKQGKDQH |
| | KRAB | FPVFMNEKEDILWCTEMERVFGFPVHYTDVSNMSRLARQRLLGRSW |
| | | SVPVIRHLFAPLKEYFACVSSGNSNANSRGPSFSSGLVPLSLRGSHMEV |
| | | KANQRNIEDICICCGSLQVHTQHPLFEGGICAPCKDKFLDALFLYDDD |
| | | GYQSYCSICCSGETLLICGNPDCTRCYCFECVDSLVGPGTSGKVHAMS |
| | | NWVCYLCLPSSRSGLLQRRRKWRSQLKAFYDRESENPLEMFETVPV |
| | | WRRQPVRVLSLFEDIKKELTSLGFLESGSDPGQLKHVVDVTDTVRKDV |
| | | EEWGPFDLVYGATPPLGHTCDRPPSWYLFQFHRLLQYARPKPGSPRP |
| | | FFWMFVDNLVLNKEDLDVASRFLEMEPVTIPDVHGGSLQNAVRVW |
| | | SNIPAIRSRHWALVSEEELSLLAQNKQSSKLAAKWPTKLVKNCFLPLRE |
| | | YFKYFSTELTSSLGGPSSGAPPPSGGSPAGSPTSTEEGTSESATPESGPG |
| | | TSTEPSEGSAPGSPAGSPTSTEEGTSTEPSEGSAPGTSTEPSEPKKKRKV |
| | | MDKKYSIGLAIGTNSVGWAVITDEYKVPSKKFKVLGNTDRHSIKKNLIG |
| | | ALLFDSGETAEATRLKRTARRRYTRRKNRICYLQEIFSNEMAKVDDSFF |
| | | HRLEESFLVEEDKKHERHPIFGNIVDEVAYHEKYPTIYHLRKKLVDSTDK |
| | | ADLRLIYLALAHMIKFRGHFLIEGDLNPDNSDVDKLFIQLVQTYNQLFE |
| | | ENPINASGVDAKAILSARLSKSRRLENLIAQLPGEKKNGLFGNLIALSLG |
| | | LTPNFKSNFDLAEDAKLQLSKDTYDDDLDNLLAQIGDQYADLFLAAKN |
| | | LSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLN |
| | | REDLLRKQRTFDNGSIPHQIHLGELHAILRRQEDFYPFLKDNREKIEKIL |
| | | TFRIPYYVGPLARGNSRFAWMTRKSEETITPWNFEEVVDKGASAQSFI |
| | | ERMTNFDKNLPNEKVLPKHSLLYEYFTVYNELTKVKYVTEGMRKPAFL |
| | | SGEQKKAIVDLLFKTNRKVTVKQLKEDYFKKIECFDSVEISGVEDRFNA |
| | | SLGTYHDLLKIIKDKDFLDNEENEDILEDIVLTLTLFEDREMIEERLKTYA |
| | | HLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDP |
| | | KKKRKVSGSETPGTSESATPESTGRTLVTFKDVFVDFTREEWKLLDTA |
| | | QQIVYRNVMLENYKNLVSLGYQLTKPDVILRLEKGEEP |
| | Fusion Protein 4 | ATGAACCATGATCAAGAATTCGACCCACCTAAAGTCTACCCACCTG |
| | DNA Sequence | TGCCCGCCGAAAAAAGGAAACCCATAAGGGTGCTGTCACTCTTTG |
| | | ATGGCATCGCCACTGGTCTCCTGGTTCTTAAGGATCTGGGAATTCA |
| | | GGTCGATCGGTACATTGCTAGCGAGGTTTGTGAGGATAGTATTAC |
| | | AGTGGGTATGGTGCGCCACCAGGGAAAGATCATGTATGTTGGTGA |
| | | CGTTAGGAGCGTCACCCAGAAACATATCCAGGAGTGGGGACCCTT |
| | | TGATTTGGTGATCGGAGGTAGTCCCTGCAATGACCTTTCCATCGTG |
| | | AATCCAGCCAGGAAAGGGCTGTATGAAGGGACTGGTAGGCTCTTT |
| | | TTCGAGTTTTATCGCCTGCTTCACGACGCTAGACCTAAGGAAGGTG |
| | | ACGATAGGCCTTTCTTTTGGCTTTTTGAGAACGTCGTGGCAATGGG |
| | | AGTCTCCGACAAAAGGGACATTTCTCGCTTTCTGGAATCTAACCCC |
| | | GTTATGATCGATGCCAAGGAAGTTTCTGCCGCTCACAGGGCAAGG |
| | | TACTTCTGGGGCAATCTGCCCGGAATGAATCGCCCACTGGCCAGTA |
| | | CCGTGAATGACAAACTGGAGCTGCAGGAGTGCCTGGAGCACGGA |
| | | AGAATCGCAAAGTTTTCTAAAGTCAGGACCATTACCACTCGCAGTA |
| | | ACTCCATAAAACAGGGTAAGGACCAGCATTTTCCCGTCTTCATGAA |
| | | TGAAAAGGAAGATATTCTGTGGTGCACTGAAATGGAGAGAGTTTT |
| | | CGGGTTTCCCGTGCACTATACCGATGTTTCCAACATGTCCCGCCTTG |
| | | CAAGACAAAGGCTTTTGGGCCGCTCTTGGTCTGTGCCAGTGATCCG |
| | | GCACTTGTTTGCTCCCCTCAAAGAGTACTTCGCTTGCGTCAGTTCCG |
| | | GAAATTCAAACGCTAACTCTCGGGGTCCATCTTTCTCCAGTGGTCT |
| | | CGTGCCACTGTCTCTCCGGGGCTCTCACATGGAAGTCAAGGCTAAC |
| | | CAGCGAAATATAGAAGACATCTGCATCTGCTGCGGAAGTCTCCAG |
| | | GTTCACACACAGCACCCTCTGTTTGAGGGAGGGATCTGCGCCCCAT |
| | | GTAAGGACAAGTTCCTGGATGCCCTCTTCCTGTACGACGATGACG |
| | | GGTACCAATCCTACTGCTCCATCTGCTGCTCCGGAGAGACGCTGCT |
| | | CATCTGCGGAAACCCTGATTGCACCCGATGCTACTGCTTCGAGTGT |
| | | GTGGATAGCCTGGTCGGCCCCGGGACCTCGGGGAAGGTGCACGC |
| | | CATGAGCAACTGGGTGTGCTACCTGTGCCTGCCGTCCTCCCGAAGC |
| | | GGGCTGCTGCAGCGTCGGAGGAAGTGGCGCAGCCAGCTCAAGGC |
| | | CTTCTACGACCGAGAGTCGGAGAATCCCCTGGAGATGTTTGAGAC |
| | | AGTGCCAGTCTGGCGGAGGCAGCCCGTTCGCGTTCTCTCTCTGTTC |
| | | GAAGATATTAAAAAGGAACTCACCTCCCTTGGGTTCCTGGAGAGC |
| | | GGGAGCGACCCCGGACAGCTTAAGCACGTGGTCGACGTGACTGA |
| | | CACCGTCCGCAAAGACGTGGAGGAATGGGGCCCCTTCGATCTGGT |
| | | CTATGGGGCAACCCCTCCCCTTGGGCATACATGTGATCGGCCTCCA |
| | | TCCTGGTACCTGTTCCAGTTTCACAGACTCCTGCAGTATGCCAGGC |
| | | CAAAGCCAGGGAGCCCAAGGCCCTTTTTCTGGATGTTCGTCGACA |
| | | ACCTGGTCCTGAACAAAGAAGATCTCGACGTTGCTAGTCGCTTTCT |
| | | CGAAATGGAGCCCGTGACCATTCCCGACGTGCATGGCGGTTCCCT |
| | | CCAGAATGCAGTCAGGGTTTGGAGCAATATCCCTGCCATCAGGTC |
| | | AAGGCACTGGGCACTGGTTTCAGAGGAAGAGCTGTCCCTCCTTGC |
| | | CCAGAACAAGCAGTCATCCAAACTGGCAGCCAAGTGGCCAACTAA |
| | | GCTGGTCAAGAACTGCTTTCTTCCCCTCAGAGAATATTTTAAGTATT |
| | | TCAGTACTGAACTGACTAGCAGTCTGGGAGGGCCGAGCTCTGGCG |
| | | CACCCCCACCAAGTGGAGGGTCTCCTGCCGGGTCCCCAACATCTAC |
| | | TGAAGAAGGCACCAGCGAATCCGCAACGCCCGAGTCAGGCCCTGG |
| | | TACCTCCACAGAACCATCTGAAGGTAGTGCGCCTGGTTCCCCAGCT |
| | | GGAAGCCCTACTTCCACCGAAGAAGGCACGTCAACCGAACCAAGT |
| | | GAAGGATCTGCCCCTGGGACCAGCACTGAACCATCTGAGCCAAAA |
| | | AAGAAGAGAAAGGTAATGGACAAGAAGTACAGCATCGGCCTGGC |
| | | CATCGGCACCAACTCTGTGGGCTGGGCCGTGATCACCGACGAGTA |
| | | CAAGGTGCCCAGCAAGAAATTCAAGGTGCTGGGCAACACCGACCG |
| | | GCACAGCATCAAGAAGAACCTGATCGGCGCCCTGCTGTTCGACAG |
| | | CGGAGAAACAGCCGAGGCCACCCGGCTGAAGAGAACCGCCAGAA |
| | | GAAGATACACCAGACGGAAGAACCGGATCTGCTATCTGCAAGAGA |
| | | TCTTCAGCAACGAGATGGCCAAGGTGGACGACAGCTTCTTCCACA |
| | | GACTGGAAGAGTCCTTCCTGGTGGAAGAGGATAAGAAGCACGAG |
| | | CGGCACCCCATCTTCGGCAACATCGTGGACGAGGTGGCCTACCAC |
| | | GAGAAGTACCCCACCATCTACCACCTGAGAAAGAAACTGGTGGAC |
| | | AGCACCGACAAGGCCGACCTGCGGCTGATCTATCTGGCCCTGGCC |
| | | CACATGATCAAGTTCCGGGGCCACTTCCTGATCGAGGGCGACCTG |
| | | AACCCCGACAACAGCGACGTGGACAAGCTGTTCATCCAGCTGGTG |
| | | CAGACCTACAACCAGCTGTTCGAGGAAAACCCCATCAACGCCAGC |
| | | GGCGTGGACGCCAAGGCCATCCTGTCTGCCAGACTGAGCAAGAGC |
| | | AGACGGCTGGAAAATCTGATCGCCCAGCTGCCCGGCGAGAAGAA |
| | | GAATGGCCTGTTCGGCAACCTGATTGCCCTGAGCCTGGGCCTGAC |
| | | CCCCAACTTCAAGAGCAACTTCGACCTGGCCGAGGATGCCAAACT |
| | | GCAGCTGAGCAAGGACACCTACGACGACGACCTGGACAACCTGCT |
| | | GGCCCAGATCGGCGACCAGTACGCCGACCTGTTTCTGGCCGCCAA |
| | | GAACCTGTCCGACGCCATCCTGCTGAGCGACATCCTGAGAGTGAA |
| | | CACCGAGATCACCAAGGCCCCCCTGAGCGCCTCTATGATCAAGAG |
| | | ATACGACGAGCACCACCAGGACCTGACCCTGCTGAAAGCTCTCGT |
| | | GCGGCAGCAGCTGCCTGAGAAGTACAAAGAGATTTTCTTCGACCA |
| | | GAGCAAGAACGGCTACGCCGGCTACATCGATGGCGGAGCCAGCC |
| | | AGGAAGAGTTCTACAAGTTCATCAAGCCCATCCTGGAAAAGATGG |
| | | ACGGCACCGAGGAACTGCTCGTGAAGCTGAACAGAGAGGACCTG |
| | | CTGCGGAAGCAGCGGACCTTCGACAACGGCAGCATCCCCCACCAG |
| | | ATCCACCTGGGAGAGCTGCACGCCATTCTGCGGCGGCAGGAAGAT |
| | | TTTTACCCATTCCTGAAGGACAACCGGGAAAAGATCGAGAAGATC |
| | | CTGACCTTCCGCATCCCCTACTACGTGGGCCCTCTGGCCAGGGGAA |
| | | ACAGCAGATTCGCCTGGATGACCAGAAAGAGCGAGGAAACCATCA |
| | | CCCCCTGGAACTTCGAGGAAGTGGTGGACAAGGGCGCCAGCGCC |
| | | CAGAGCTTCATCGAGCGGATGACCAACTTCGATAAGAACCTGCCC |
| | | AACGAGAAGGTGCTGCCCAAGCACAGCCTGCTGTACGAGTACTTC |
| | | ACCGTGTACAACGAGCTGACCAAAGTGAAATACGTGACCGAGGGA |
| | | ATGAGAAAGCCCGCCTTCCTGAGCGGCGAGCAGAAAAAAGCCATC |
| | | GTGGACCTGCTGTTCAAGACCAACCGGAAAGTGACCGTGAAGCAG |
| | | CTGAAAGAGGACTACTTCAAGAAAATCGAGTGCTTCGACTCCGTG |
| | | GAAATCTCCGGCGTGGAAGATCGGTTCAACGCCTCCCTGGGCACA |
| | | TACCACGATCTGCTGAAAATTATCAAGGACAAGGACTTCCTGGACA |
| | | ATGAGGAAAACGAGGACATTCTGGAAGATATCGTGCTGACCCTGA |
| | | CACTGTTTGAGGACAGAGAGATGATCGAGGAACGGCTGAAAACCT |
| | | ATGCCCACCTGTTCGACGACAAAGTGATGAAGCAGCTGAAGCGGC |
| | | GGAGATACACCGGCTGGGGCAGGCTGAGCCGGAAGCTGATCAAC |
| | | GGCATCCGGGACAAGCAGTCCGGCAAGACAATCCTGGATTTCCTG |
| | | AAGTCCGACGGCTTCGCCAACAGAAACTTCATGCAGCTGATCCAC |
| | | GACGACAGCCTGACCTTTAAAGAGGACATCCAGAAAGCCCAGGTG |
| | | TCCGGCCAGGGCGATAGCCTGCACGAGCACATTGCCAATCTGGCC |
| | | GGCAGCCCCGCCATTAAGAAGGGCATCCTGCAGACAGTGAAGGT |
| | | GGTGGACGAGCTCGTGAAAGTGATGGGCCGGCACAAGCCCGAGA |
| | | ACATCGTGATCGAAATGGCCAGAGAGAACCAGACCACCCAGAAG |
| | | GGACAGAAGAACAGCCGCGAGAGAATGAAGCGGATCGAAGAGG |
| | | GCATCAAAGAGCTGGGCAGCCAGATCCTGAAAGAACACCCCGTGG |
| | | AAAACACCCAGCTGCAGAACGAGAAGCTGTACCTGTACTACCTGC |
| | | AGAATGGGCGGGATATGTACGTGGACCAGGAACTGGACATCAAC |
| | | CGGCTGTCCGACTACGATGTGGACGCTATCGTGCCTCAGAGCTTTC |
| | | TGAAGGACGACTCCATCGATAACAAAGTGCTGACTCGGAGCGACA |
| | | AGAACCGGGGCAAGAGCGACAACGTGCCCTCCGAAGAGGTCGTG |
| | | AAGAAGATGAAGAACTACTGGCGCCAGCTGCTGAATGCCAAGCTG |
| | | ATTACCCAGAGGAAGTTCGACAATCTGACCAAGGCCGAGAGAGGC |
| | | GGCCTGAGCGAACTGGATAAGGCCGGCTTCATCAAGAGACAGCTG |
| | | GTGGAAACCCGGCAGATCACAAAGCACGTGGCACAGATCCTGGAC |
| | | TCCCGGATGAACACTAAGTACGACGAGAACGACAAACTGATCCGG |
| | | GAAGTGAAAGTGATCACCCTGAAGTCCAAGCTGGTGTCCGATTTC |
| | | CGGAAGGATTTCCAGTTTTACAAAGTGCGCGAGATCAACAACTAC |
| | | CACCACGCCCACGACGCCTACCTGAACGCCGTCGTGGGAACCGCC |
| | | CTGATCAAAAAGTACCCTAAGCTGGAAAGCGAGTTCGTGTACGGC |
| | | GACTACAAGGTGTACGACGTGCGGAAGATGATCGCCAAGAGCGA |
| | | GCAGGAAATCGGCAAGGCTACCGCCAAGTACTTCTTCTACAGCAA |
| | | CATCATGAACTTTTTCAAGACCGAGATTACCCTGGCCAACGGCGAG |
| | | ATCCGGAAGCGGCCTCTGATCGAGACAAACGGCGAAACAGGCGA |
| | | GATCGTGTGGGATAAGGGCCGGGACTTTGCCACCGTGCGGAAAG |
| | | TGCTGTCTATGCCCCAAGTGAATATCGTGAAAAAGACCGAGGTGC |
| | | AGACAGGCGGCTTCAGCAAAGAGTCTATCCTGCCCAAGAGGAACA |
| | | GCGACAAGCTGATCGCCAGAAAGAAGGACTGGGACCCTAAGAAG |
| | | TACGGCGGCTTCGACAGCCCCACCGTGGCCTATTCTGTGCTGGTGG |
| | | TGGCCAAAGTGGAAAAGGGCAAGTCCAAGAAACTGAAGAGTGTG |
| | | AAAGAGCTGCTGGGGATCACCATCATGGAAAGAAGCAGCTTCGAG |
| | | AAGAATCCCATCGACTTTCTGGAAGCCAAGGGCTACAAAGAAGTG |
| | | AAAAAGGACCTGATCATCAAGCTGCCTAAGTACTCCCTGTTCGAGC |
| | | TGGAAAACGGCCGGAAGAGAATGCTGGCCTCTGCCGGCGAACTG |
| | | CAGAAGGGAAACGAACTGGCCCTGCCCTCCAAATATGTGAACTTC |
| | | CTGTACCTGGCCAGCCACTATGAGAAGCTGAAGGGCTCCCCCGAG |
| | | GATAATGAGCAGAAACAGCTGTTTGTGGAACAGCACAAACACTAC |
| | | CTGGACGAGATCATCGAGCAGATCAGCGAGTTCTCCAAGAGAGTG |
| | | ATCCTGGCCGACGCTAATCTGGACAAGGTGCTGAGCGCCTACAAC |
| | | AAGCACAGAGACAAGCCTATCAGAGAGCAGGCCGAGAATATCATC |
| | | CACCTGTTTACCCTGACCAATCTGGGAGCCCCTGCCGCCTTCAAGT |
| | | ACTTTGACACCACCATCGACCGGAAGAGGTACACCAGCACCAAAG |
| | | AGGTGCTGGACGCCACCCTGATCCACCAGAGCATCACCGGCCTGT |
| | | ACGAGACACGGATCGACCTGTCTCAGCTGGGAGGCGACCCAAAAA |
| | | AGAAGAGAAAGGTAAGCGGAAGTGAGACCCCAGGTACATCCGAA |
| | | TCAGCAACGCCTGAAAGCACCGGTCGGACACTGGTGACCTTCAAG |
| | | GATGTATTTGTGGACTTCACCAGGGAGGAGTGGAAGCTGCTGGAC |
| | | ACTGCTCAGCAGATCGTGTACAGAAATGTGATGCTGGAGAACTAT |
| | | AAGAACCTGGTTTCCTTGGGTTATCAGCTTACTAAGCCAGATGTGA |
| | | TCCTCCGGTTGGAGAAGGGAGAAGAGCCC |
| | Fusion Protein 5 | MPKKKRKVPKKKRKVNHDQEFDPPKVYPPVPAEKRKPIRVLSLFDGIA |
| | Amino Acid | TGLLVLKDLGIQVDRYIASEVCEDSITVGMVRHQGKIMYVGDVRSVT |
| | Sequence | QKHIQEWGPFDLVIGGSPCNDLSIVNPARKGLYEGTGRLFFEFYRLLH |
| | NLS-NLS-3A-3L- | DARPKEGDDRPFFWLFENVVAMGVSDKRDISRFLESNPVMIDAKEVS |
| | dCas9-KRAB- | AAHRARYFWGNLPGMNRPLASTVNDKLELQECLEHGRIAKFSKVRTI |
| | NLS-NLS | TTRSNSIKQGKDQHFPVFMNEKEDILWCTEMERVFGFPVHYTDVSN |
| | | MSRLARQRLLGRSWSVPVIRHLFAPLKEYFACVSSGNSNANSRGPSFS |
| | | SGLVPLSLRGSHNPLEMFETVPVWRRQPVRVLSLFEDIKKELTSLGFLE |
| | | SGSDPGQLKHVVDVTDTVRKDVEEWGPFDLVYGATPPLGHTCDRPP |
| | | SWYLFQFHRLLQYARPKPGSPRPFFWMFVDNLVLNKEDLDVASRFLE |
| | | MEPVTIPDVHGGSLQNAVRVWSNIPAIRSRHWALVSEEELSLLAQNK |
| | | QSSKLAAKWPTKLVKNCFLPLREYFKYFSTELTSSLGGPSSGAPPPSGG |
| | | SPAGSPTSTEEGTSESATPESGPGTSTEPSEGSAPGSPAGSPTSTEEGT |
| | | STEPSEGSAPGTSTEPSEMDKKYSIGLAIGTNSVGWAVITDEYKVPSKK |
| | | FKVLGNTDRHSIKKNLIGALLFDSGETAEATRLKRTARRRYTRRKNRICY |
| | | LQEIFSNEMAKVDDSFFHRLEESFLVEEDKKHERHPIFGNIVDEVAYHE |
| | | KYPTIYHLRKKLVDSTDKADLRLIYLALAHMIKFRGHFLIEGDLNPDNSD |
| | | VDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLL |
| | | AQIGDQYADLFLAAKNLSDAILLSDILRVNTEITKAPLSASMIKRYDEHH |
| | | QDLTLLKALVRQQLPEKYKEIFFDQSKNGYAGYIDGGASQEEFYKFIKPI |
| | | LEKMDGTEELLVKLNREDLLRKQRTFDNGSIPHQIHLGELHAILRRQED |
| | | FYPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKIECF |
| | | DSVEISGVEDRFNASLGTYHDLLKIIKDKDFLDNEENEDILEDIVLTLTLF |
| | | EDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSL |
| | | HEHIANLAGSPAIKKGILQTVKVVDELVKVMGRHKPENIVIEMARENQ |
| | | TTQKGQKNSRERMKRIEEGIKELGSQILKEHPVENTQLQNEKLYLYYLQ |
| | | NGRDMYVDQELDINRLSDYDVDAIVPQSFLKDDSIDNKVLTRSDKNR |
| | | GKSDNVPSEEVVKKMKNYWRQLLNAKLITQRKFDNLTKAERGGLSEL |
| | | DKAGFIKRQLVETRQITKHVAQILDSRMNTKYDENDKLIREVKVITLKS |
| | | KLVSDFRKDFQFYKVREINNYHHAHDAYLNAVVGTALIKKYPKLESEFV |
| | | YGDYKVYDVRKMIAKSEQEIGKATAKYFFYSNIMNFFKTEITLANGEIR |
| | | KRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKS |
| | | KKLKSVKELLGITIMERSSFEKNPIDFLEAKGYKEVKKDLIIKLPKYSLFELE |
| | | NGRKRMLASAGELQKGNELALPSKYVNFLYLASHYEKLKGSPEDNEQ |
| | | KQLFVEQHKHYLDEIIEQISEFSKRVILADANLDKVLSAYNKHRDKPIRE |
| | | QAENIIHLFTLTNLGAPAAFKYFDTTIDRKRYTSTKEVLDATLIHQSITGL |
| | | YETRIDLSQLGGDSGSETPGTSESATPESTGRTLVTFKDVFVDFTREEW |
| | | KLLDTAQQIVYRNVMLENYKNLVSLGYQLTKPDVILRLEKGEEPSADYK |
| | | DDDDKAPKKKRKVPKKKRKV |
| | Fusion Protein 5 | ATGCCAAAAAAGAAGAGAAAGGTACCGAAGAAAAAAAGAAAGGT |
| | DNA Sequence | CAACCACGACCAGGAATTCGACCCTCCAAAGGTTTACCCACCTGTC |
| | | CCAGCTGAGAAGAGGAAGCCCATCCGGGTGCTGTCTCTCTTTGAT |
| | | GGAATCGCTACAGGGCTCCTGGTGCTGAAGGACTTGGGCATTCAG |
| | | GTGGACCGCTACATTGCCTCGGAGGTGTGTGAGGACTCCATCACG |
| | | GTGGGCATGGTGCGGCACCAGGGGAAGATCATGTACGTCGGGGA |
| | | CGTCCGCAGCGTCACACAGAAGCATATCCAGGAGTGGGGCCCATT |
| | | CGATCTGGTGATTGGGGGCAGTCCCTGCAATGACCTCTCCATCGTC |
| | | AACCCTGCTCGCAAGGGCCTCTACGAGGGCACTGGCCGGCTCTTCT |
| | | TTGAGTTCTACCGCCTCCTGCATGATGCGCGGCCCAAGGAGGGAG |
| | | ATGATCGCCCCTTCTTCTGGCTCTTTGAGAATGTGGTGGCCATGGG |
| | | CGTTAGTGACAAGAGGGACATCTCGCGATTTCTCGAGTCCAACCCT |
| | | GTGATGATTGATGCCAAAGAAGTGTCAGCTGCACACAGGGCCCGC |
| | | TACTTCTGGGGTAACCTTCCCGGTATGAACAGGCCGTTGGCATCCA |
| | | CTGTGAATGATAAGCTGGAGCTGCAGGAGTGTCTGGAGCATGGCA |
| | | GGATAGCCAAGTTCAGCAAAGTGAGGACCATTACTACGAGGTCAA |
| | | ACTCCATAAAGCAGGGCAAAGACCAGCATTTTCCTGTCTTCATGAA |
| | | TGAGAAAGAGGACATCTTATGGTGCACTGAAATGGAAAGGGTATT |
| | | TGGTTTCCCAGTCCACTATACTGACGTCTCCAACATGAGCCGCTTG |
| | | GCGAGGCAGAGACTGCTGGGCCGGTCATGGAGCGTGCCAGTCAT |
| | | CCGCCACCTCTTCGCTCCGCTGAAGGAGTATTTTGCGTGTGTGTCT |
| | | AGCGGCAATAGTAACGCTAACAGCCGCGGGCCGAGCTTCAGCAGC |
| | | GGCCTGGTGCCGTTAAGCTTGCGCGGCAGCCATAATCCCCTTGAG |
| | | ATGTTCGAAACCGTGCCTGTGTGGAGGAGACAGCCAGTCCGGGTG |
| | | CTGTCCCTTTTTGAAGACATCAAGAAAGAGCTGACGAGTTTGGGCT |
| | | TTTTGGAAAGTGGTTCTGACCCGGGACAACTGAAGCATGTGGTTG |
| | | ATGTCACAGACACAGTGAGGAAGGATGTGGAGGAGTGGGGACCC |
| | | TTCGATCTTGTGTACGGCGCCACACCTCCCCTGGGCCACACCTGTG |
| | | ACCGTCCTCCCAGCTGGTACCTGTTCCAGTTCCACCGGCTCCTGCA |
| | | GTACGCACGGCCCAAGCCAGGCAGCCCCAGGCCCTTCTTCTGGAT |
| | | GTTCGTGGACAATCTGGTGCTGAACAAGGAAGACCTGGACGTCGC |
| | | ATCTCGCTTCCTGGAGATGGAGCCAGTCACCATCCCAGATGTCCAC |
| | | GGCGGATCCTTGCAGAATGCTGTCCGCGTGTGGAGCAACATCCCA |
| | | GCCATAAGGAGCAGGCACTGGGCTCTGGTTTCGGAAGAAGAATTG |
| | | TCCCTGCTGGCCCAGAACAAGCAGAGCTCGAAGCTCGCGGCCAAG |
| | | TGGCCCACCAAGCTGGTGAAGAACTGCTTTCTCCCCCTAAGAGAAT |
| | | ATTTCAAGTATTTTTCAACAGAACTCACTTCCTCTTTAGGAGGGCCG |
| | | AGCTCTGGCGCACCCCCACCAAGTGGAGGGTCTCCTGCCGGGTCC |
| | | CCAACATCTACTGAAGAAGGCACCAGCGAATCCGCAACGCCCGAG |
| | | TCAGGCCCTGGTACCTCCACAGAACCATCTGAAGGTAGTGCGCCT |
| | | GGTTCCCCAGCTGGAAGCCCTACTTCCACCGAAGAAGGCACGTCA |
| | | ACCGAACCAAGTGAAGGATCTGCCCCTGGGACCAGCACTGAACCA |
| | | TCTGAGATGGACAAGAAGTACAGCATCGGCCTGGCCATCGGCACC |
| | | AACTCTGTGGGCTGGGCCGTGATCACCGACGAGTACAAGGTGCCC |
| | | AGCAAGAAATTCAAGGTGCTGGGCAACACCGACCGGCACAGCATC |
| | | AAGAAGAACCTGATCGGCGCCCTGCTGTTCGACAGCGGAGAAACA |
| | | GCCGAGGCCACCCGGCTGAAGAGAACCGCCAGAAGAAGATACAC |
| | | CAGACGGAAGAACCGGATCTGCTATCTGCAAGAGATCTTCAGCAA |
| | | CGAGATGGCCAAGGTGGACGACAGCTTCTTCCACAGACTGGAAGA |
| | | GTCCTTCCTGGTGGAAGAGGATAAGAAGCACGAGCGGCACCCCAT |
| | | CTTCGGCAACATCGTGGACGAGGTGGCCTACCACGAGAAGTACCC |
| | | CACCATCTACCACCTGAGAAAGAAACTGGTGGACAGCACCGACAA |
| | | GGCCGACCTGCGGCTGATCTATCTGGCCCTGGCCCACATGATCAA |
| | | GTTCCGGGGCCACTTCCTGATCGAGGGCGACCTGAACCCCGACAA |
| | | CAGCGACGTGGACAAGCTGTTCATCCAGCTGGTGCAGACCTACAA |
| | | CCAGCTGTTCGAGGAAAACCCCATCAACGCCAGCGGCGTGGACGC |
| | | CAAGGCCATCCTGTCTGCCAGACTGAGCAAGAGCAGACGGCTGGA |
| | | AAATCTGATCGCCCAGCTGCCCGGCGAGAAGAAGAATGGCCTGTT |
| | | CGGCAACCTGATTGCCCTGAGCCTGGGCCTGACCCCCAACTTCAAG |
| | | AGCAACTTCGACCTGGCCGAGGATGCCAAACTGCAGCTGAGCAAG |
| | | GACACCTACGACGACGACCTGGACAACCTGCTGGCCCAGATCGGC |
| | | GACCAGTACGCCGACCTGTTTCTGGCCGCCAAGAACCTGTCCGAC |
| | | GCCATCCTGCTGAGCGACATCCTGAGAGTGAACACCGAGATCACC |
| | | AAGGCCCCCCTGAGCGCCTCTATGATCAAGAGATACGACGAGCAC |
| | | CACCAGGACCTGACCCTGCTGAAAGCTCTCGTGCGGCAGCAGCTG |
| | | CCTGAGAAGTACAAAGAGATTTTCTTCGACCAGAGCAAGAACGGC |
| | | TACGCCGGCTACATCGATGGCGGAGCCAGCCAGGAAGAGTTCTAC |
| | | AAGTTCATCAAGCCCATCCTGGAAAAGATGGACGGCACCGAGGAA |
| | | CTGCTCGTGAAGCTGAACAGAGAGGACCTGCTGCGGAAGCAGCG |
| | | GACCTTCGACAACGGCAGCATCCCCCACCAGATCCACCTGGGAGA |
| | | GCTGCACGCCATTCTGCGGCGGCAGGAAGATTTTTACCCATTCCTG |
| | | AAGGACAACCGGGAAAAGATCGAGAAGATCCTGACCTTCCGCATC |
| | | CCCTACTACGTGGGCCCTCTGGCCAGGGGAAACAGCAGATTCGCC |
| | | TGGATGACCAGAAAGAGCGAGGAAACCATCACCCCCTGGAACTTC |
| | | GAGGAAGTGGTGGACAAGGGCGCCAGCGCCCAGAGCTTCATCGA |
| | | GCGGATGACCAACTTCGATAAGAACCTGCCCAACGAGAAGGTGCT |
| | | GCCCAAGCACAGCCTGCTGTACGAGTACTTCACCGTGTACAACGA |
| | | GCTGACCAAAGTGAAATACGTGACCGAGGGAATGAGAAAGCCCG |
| | | CCTTCCTGAGCGGCGAGCAGAAAAAAGCCATCGTGGACCTGCTGT |
| | | TCAAGACCAACCGGAAAGTGACCGTGAAGCAGCTGAAAGAGGAC |
| | | TACTTCAAGAAAATCGAGTGCTTCGACTCCGTGGAAATCTCCGGCG |
| | | TGGAAGATCGGTTCAACGCCTCCCTGGGCACATACCACGATCTGCT |
| | | GAAAATTATCAAGGACAAGGACTTCCTGGACAATGAGGAAAACGA |
| | | GGACATTCTGGAAGATATCGTGCTGACCCTGACACTGTTTGAGGA |
| | | CAGAGAGATGATCGAGGAACGGCTGAAAACCTATGCCCACCTGTT |
| | | CGACGACAAAGTGATGAAGCAGCTGAAGCGGCGGAGATACACCG |
| | | GCTGGGGCAGGCTGAGCCGGAAGCTGATCAACGGCATCCGGGAC |
| | | AAGCAGTCCGGCAAGACAATCCTGGATTTCCTGAAGTCCGACGGC |
| | | TTCGCCAACAGAAACTTCATGCAGCTGATCCACGACGACAGCCTGA |
| | | CCTTTAAAGAGGACATCCAGAAAGCCCAGGTGTCCGGCCAGGGCG |
| | | ATAGCCTGCACGAGCACATTGCCAATCTGGCCGGCAGCCCCGCCA |
| | | TTAAGAAGGGCATCCTGCAGACAGTGAAGGTGGTGGACGAGCTC |
| | | GTGAAAGTGATGGGCCGGCACAAGCCCGAGAACATCGTGATCGA |
| | | AATGGCCAGAGAGAACCAGACCACCCAGAAGGGACAGAAGAACA |
| | | GCCGCGAGAGAATGAAGCGGATCGAAGAGGGCATCAAAGAGCTG |
| | | GGCAGCCAGATCCTGAAAGAACACCCCGTGGAAAACACCCAGCTG |
| | | CAGAACGAGAAGCTGTACCTGTACTACCTGCAGAATGGGCGGGAT |
| | | ATGTACGTGGACCAGGAACTGGACATCAACCGGCTGTCCGACTAC |
| | | GATGTGGACGCTATCGTGCCTCAGAGCTTTCTGAAGGACGACTCC |
| | | ATCGATAACAAAGTGCTGACTCGGAGCGACAAGAACCGGGGCAA |
| | | GAGCGACAACGTGCCCTCCGAAGAGGTCGTGAAGAAGATGAAGA |
| | | ACTACTGGCGCCAGCTGCTGAATGCCAAGCTGATTACCCAGAGGA |
| | | AGTTCGACAATCTGACCAAGGCCGAGAGAGGCGGCCTGAGCGAA |
| | | CTGGATAAGGCCGGCTTCATCAAGAGACAGCTGGTGGAAACCCGG |
| | | CAGATCACAAAGCACGTGGCACAGATCCTGGACTCCCGGATGAAC |
| | | ACTAAGTACGACGAGAACGACAAACTGATCCGGGAAGTGAAAGT |
| | | GATCACCCTGAAGTCCAAGCTGGTGTCCGATTTCCGGAAGGATTTC |
| | | CAGTTTTACAAAGTGCGCGAGATCAACAACTACCACCACGCCCACG |
| | | ACGCCTACCTGAACGCCGTCGTGGGAACCGCCCTGATCAAAAAGT |
| | | ACCCTAAGCTGGAAAGCGAGTTCGTGTACGGCGACTACAAGGTGT |
| | | ACGACGTGCGGAAGATGATCGCCAAGAGCGAGCAGGAAATCGGC |
| | | AAGGCTACCGCCAAGTACTTCTTCTACAGCAACATCATGAACTTTTT |
| | | CAAGACCGAGATTACCCTGGCCAACGGCGAGATCCGGAAGCGGCC |
| | | TCTGATCGAGACAAACGGCGAAACAGGCGAGATCGTGTGGGATA |
| | | AGGGCCGGGACTTTGCCACCGTGCGGAAAGTGCTGTCTATGCCCC |
| | | AAGTGAATATCGTGAAAAAGACCGAGGTGCAGACAGGCGGCTTC |
| | | AGCAAAGAGTCTATCCTGCCCAAGAGGAACAGCGACAAGCTGATC |
| | | GCCAGAAAGAAGGACTGGGACCCTAAGAAGTACGGCGGCTTCGA |
| | | CAGCCCCACCGTGGCCTATTCTGTGCTGGTGGTGGCCAAAGTGGA |
| | | AAAGGGCAAGTCCAAGAAACTGAAGAGTGTGAAAGAGCTGCTGG |
| | | GGATCACCATCATGGAAAGAAGCAGCTTCGAGAAGAATCCCATCG |
| | | ACTTTCTGGAAGCCAAGGGCTACAAAGAAGTGAAAAAGGACCTGA |
| | | TCATCAAGCTGCCTAAGTACTCCCTGTTCGAGCTGGAAAACGGCCG |
| | | GAAGAGAATGCTGGCCTCTGCCGGCGAACTGCAGAAGGGAAACG |
| | | AACTGGCCCTGCCCTCCAAATATGTGAACTTCCTGTACCTGGCCAG |
| | | CCACTATGAGAAGCTGAAGGGCTCCCCCGAGGATAATGAGCAGAA |
| | | ACAGCTGTTTGTGGAACAGCACAAACACTACCTGGACGAGATCAT |
| | | CGAGCAGATCAGCGAGTTCTCCAAGAGAGTGATCCTGGCCGACGC |
| | | TAATCTGGACAAGGTGCTGAGCGCCTACAACAAGCACAGAGACAA |
| | | GCCTATCAGAGAGCAGGCCGAGAATATCATCCACCTGTTTACCCTG |
| | | ACCAATCTGGGAGCCCCTGCCGCCTTCAAGTACTTTGACACCACCA |
| | | TCGACCGGAAGAGGTACACCAGCACCAAAGAGGTGCTGGACGCC |
| | | ACCCTGATCCACCAGAGCATCACCGGCCTGTACGAGACACGGATC |
| | | GACCTGTCTCAGCTGGGAGGCGACAGCGGAAGTGAGACCCCAGG |
| | | TACATCCGAATCAGCAACGCCTGAAAGCACCGGTCGGACACTGGT |
| | | GACCTTCAAGGATGTATTTGTGGACTTCACCAGGGAGGAGTGGAA |
| | | GCTGCTGGACACTGCTCAGCAGATCGTGTACAGAAATGTGATGCT |
| | | GGAGAACTATAAGAACCTGGTTTCCTTGGGTTATCAGCTTACTAAG |
| | | CCAGATGTGATCCTCCGGTTGGAGAAGGGAGAAGAGCCCAGCGC |
| | | TGATTACAAAGATGATGACGATAAAGCCCCAAAAAAGAAGAGAAA |
| | | GGTACCGAAGAAAAAAAGAAAGGTC |
| | Fusion Protein 6 | MPKKKRKVPKKKRKVNHDQEFDPPKVYPPVPAEKRKPIRVLSLFDGIA |
| | Amino Acid | TGLLVLKDLGIQVDRYIASEVCEDSITVGMVRHQGKIMYVGDVRSVT |
| | Sequence | QKHIQEWGPFDLVIGGSPCNDLSIVNPARKGLYEGTGRLFFEFYRLLH |
| | NLS-NLS-3A-3L- | DARPKEGDDRPFFWLFENVVAMGVSDKRDISRFLESNPVMIDAKEVS |
| | dCas9-KRAB- | AAHRARYFWGNLPGMNRPLASTVNDKLELQECLEHGRIAKFSKVRTI |
| | NLS-NLS | TTRSNSIKQGKDQHFPVFMNEKEDILWCTEMERVFGFPVHYTDVSN |
| | | MSRLARQRLLGRSWSVPVIRHLFAPLKEYFACVSSGNSNANSRGPSFS |
| | | SGLVPLSLRGSHMGPMEIYKTVSAWKRQPVRVLSLFRNIDKVLKSLGF |
| | | LESGSGSGGGTLKYVEDVTNVVRRDVEKWGPFDLVYGSTQPLGSSCD |
| | | RCPGWYMFQFHRILQYALPRQESQRPFFWIFMDNLLLTEDDQETTT |
| | | RFLQTEAVTLQDVRGRDYQNAMRVWSNIPGLKSKHAPLTPKEEEYLQ |
| | | AQVRSRSKLDAPKVDLLVKNCLLPLREYFKYFSQNSLPLGGPSSGAPPP |
| | | SGGSPAGSPTSTEEGTSESATPESGPGTSTEPSEGSAPGSPAGSPTSTE |
| | | EGTSTEPSEGSAPGTSTEPSEMDKKYSIGLAIGTNSVGWAVITDEYKVP |
| | | SKKFKVLGNTDRHSIKKNLIGALLFDSGETAEATRLKRTARRRYTRRKN |
| | | RICYLQEIFSNEMAKVDDSFFHRLEESFLVEEDKKHERHPIFGNIVDEV |
| | | AYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMIKR |
| | | YDEHHQDLTLLKALVRQQLPEKYKEIFFDQSKNGYAGYIDGGASQEEF |
| | | YKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEE |
| | | TITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDILED |
| | | IVLTLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEM |
| | | ARENQTTQKGQKNSRERMKRIEEGIKELGSQILKEHPVENTQLQNEKL |
| | | YLYYLQNGRDMYVDQELDINRLSDYDVDAIVPQSFLKDDSIDNKVLTR |
| | | SDKNRGKSDNVPSEEVVKKMKNYWRQLLNAKLITQRKFDNLTKAER |
| | | GGLSELDKAGFIKRQLVETRQITKHVAQILDSRMNTKYDENDKLIREVK |
| | | VITLKSKLVSDFRKDFQFYKVREINNYHHAHDAYLNAVVGTALIKKYPK |
| | | LESEFVYGDYKVYDVRKMIAKSEQEIGKATAKYFFYSNIMNFFKTEITLA |
| | | NGEIRKRPLIETNGETGEIVWDKGRDFATVRKVLSMPQVNIVKKTEVQ |
| | | TGGFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKY |
| | | SLFELENGRKRMLASAGELQKGNELALPSKYVNFLYLASHYEKLKGSPE |
| | | DNEQKQLFVEQHKHYLDEIIEQISEFSKRVILADANLDKVLSAYNKHRD |
| | | KPIREQAENIIHLFTLTNLGAPAAFKYFDTTIDRKRYTSTKEVLDATLIHQ |
| | | SITGLYETRIDLSQLGGDSGSETPGTSESATPESTGRTLVTFKDVFVDFT |
| | | REEWKLLDTAQQIVYRNVMLENYKNLVSLGYQLTKPDVILRLEKGEEP |
| | | SADYKDDDDKAPKKKRKVPKKKRKV |
| | Fusion Protein 6 | ATGCCAAAAAAGAAGAGAAAGGTACCGAAGAAAAAAAGAAAGGT |
| | DNA Sequence | ATACAACCATGACCAGGAATTCGACCCCCCAAAGGTTTACCCACCT |
| | | GTGCCAGCTGAGAAGAGGAAGCCCATCCGCGTGCTGTCTCTCTTT |
| | | GATGGGATTGCTACAGGGCTCCTGGTGCTGAAGGACCTGGGCATC |
| | | CAAGTGGACCGCTACATTGCCTCCGAGGTGTGTGAGGACTCCATC |
| | | ACGGTGGGCATGGTGCGGCACCAGGGAAAGATCATGTACGTCGG |
| | | GGACGTCCGCAGCGTCACACAGAAGCATATCCAGGAGTGGGGCC |
| | | CATTCGACCTGGTGATTGGAGGCAGTCCCTGCAATGACCTCTCCAT |
| | | TGTCAACCCTGCCCGCAAGGGACTTTATGAGGGTACTGGCCGCCTC |
| | | TTCTTTGAGTTCTACCGCCTCCTGCATGATGCGCGGCCCAAGGAGG |
| | | GAGATGATCGCCCCTTCTTCTGGCTCTTTGAGAATGTGGTGGCCAT |
| | | GGGCGTTAGTGACAAGAGGGACATCTCGCGATTTCTTGAGTCTAA |
| | | CCCCGTGATGATTGACGCCAAAGAAGTGTCTGCTGCACACAGGGC |
| | | CCGTTACTTCTGGGGTAACCTTCCTGGCATGAACAGGCCTTTGGCA |
| | | TCCACTGTGAATGATAAGCTGGAGCTGCAAGAGTGTCTGGAGCAC |
| | | GGCAGAATAGCCAAGTTCAGCAAAGTGAGGACCATTACCACCAGG |
| | | TCAAACTCTATAAAGCAGGGCAAAGACCAGCATTTCCCCGTCTTCA |
| | | TGAACGAGAAGGAGGACATCCTGTGGTGCACTGAAATGGAAAGG |
| | | GTGTTTGGCTTCCCCGTCCACTACACAGACGTCTCCAACATGAGCC |
| | | GCTTGGCGAGGCAGAGACTGCTGGGCCGATCGTGGAGCGTGCCG |
| | | GTCATCCGCCACCTCTTCGCTCCGCTGAAGGAATATTTTGCTTGTGT |
| | | GTCTAGCGGCAATAGTAACGCTAACAGCCGCGGGCCGAGCTTCAG |
| | | CAGCGGCCTGGTGCCGTTAAGCTTGCGCGGCAGCCATATGGGCCC |
| | | TATGGAGATATACAAGACAGTGTCTGCATGGAAGAGACAGCCAGT |
| | | GCGGGTACTGAGCCTCTTCAGAAACATCGACAAGGTACTAAAGAG |
| | | TTTGGGCTTCTTGGAAAGCGGTTCTGGTTCTGGGGGAGGAACGCT |
| | | GAAGTACGTGGAAGATGTCACAAATGTCGTGAGGAGAGACGTGG |
| | | AGAAATGGGGCCCCTTTGACCTGGTGTACGGCTCGACGCAGCCCC |
| | | TAGGCAGCTCTTGTGATCGCTGTCCCGGCTGGTACATGTTCCAGTT |
| | | CCACCGGATCCTGCAGTATGCGCTGCCTCGCCAGGAGAGTCAGCG |
| | | GCCCTTCTTCTGGATATTCATGGACAATCTGCTGCTGACTGAGGAT |
| | | GACCAAGAGACAACTACCCGCTTCCTTCAGACAGAGGCTGTGACC |
| | | CTCCAGGATGTCCGTGGCAGAGACTACCAGAATGCTATGCGGGTG |
| | | TGGAGCAACATTCCAGGGCTGAAGAGCAAGCATGCGCCCCTGACC |
| | | CCAAAGGAAGAAGAGTATCTGCAAGCCCAAGTCAGAAGCAGGAG |
| | | CAAGCTGGACGCCCCGAAAGTTGACCTCCTGGTGAAGAACTGCCT |
| | | TCTCCCGCTGAGAGAGTACTTCAAGTATTTTTCTCAAAACTCACTTC |
| | | CTCTTGGAGGGCCGAGCTCTGGCGCACCCCCACCAAGTGGAGGGT |
| | | CTCCTGCCGGGTCCCCAACATCTACTGAAGAAGGCACCAGCGAAT |
| | | CCGCAACGCCCGAGTCAGGCCCTGGTACCTCCACAGAACCATCTG |
| | | AAGGTAGTGCGCCTGGTTCCCCAGCTGGAAGCCCTACTTCCACCGA |
| | | AGAAGGCACGTCAACCGAACCAAGTGAAGGATCTGCCCCTGGGAC |
| | | CAGCACTGAACCATCTGAGATGGACAAGAAGTACAGCATCGGCCT |
| | | GGCCATCGGCACCAACTCTGTGGGCTGGGCCGTGATCACCGACGA |
| | | GTACAAGGTGCCCAGCAAGAAATTCAAGGTGCTGGGCAACACCGA |
| | | CCGGCACAGCATCAAGAAGAACCTGATCGGCGCCCTGCTGTTCGA |
| | | CAGCGGAGAAACAGCCGAGGCCACCCGGCTGAAGAGAACCGCCA |
| | | GAAGAAGATACACCAGACGGAAGAACCGGATCTGCTATCTGCAAG |
| | | AGATCTTCAGCAACGAGATGGCCAAGGTGGACGACAGCTTCTTCC |
| | | ACAGACTGGAAGAGTCCTTCCTGGTGGAAGAGGATAAGAAGCAC |
| | | GAGCGGCACCCCATCTTCGGCAACATCGTGGACGAGGTGGCCTAC |
| | | CACGAGAAGTACCCCACCATCTACCACCTGAGAAAGAAACTGGTG |
| | | GACAGCACCGACAAGGCCGACCTGCGGCTGATCTATCTGGCCCTG |
| | | GCCCACATGATCAAGTTCCGGGGCCACTTCCTGATCGAGGGCGAC |
| | | CTGAACCCCGACAACAGCGACGTGGACAAGCTGTTCATCCAGCTG |
| | | GTGCAGACCTACAACCAGCTGTTCGAGGAAAACCCCATCAACGCC |
| | | AGCGGCGTGGACGCCAAGGCCATCCTGTCTGCCAGACTGAGCAAG |
| | | AGCAGACGGCTGGAAAATCTGATCGCCCAGCTGCCCGGCGAGAA |
| | | GAAGAATGGCCTGTTCGGCAACCTGATTGCCCTGAGCCTGGGCCT |
| | | GACCCCCAACTTCAAGAGCAACTTCGACCTGGCCGAGGATGCCAA |
| | | ACTGCAGCTGAGCAAGGACACCTACGACGACGACCTGGACAACCT |
| | | GCTGGCCCAGATCGGCGACCAGTACGCCGACCTGTTTCTGGCCGC |
| | | CAAGAACCTGTCCGACGCCATCCTGCTGAGCGACATCCTGAGAGT |
| | | GAACACCGAGATCACCAAGGCCCCCCTGAGCGCCTCTATGATCAA |
| | | GAGATACGACGAGCACCACCAGGACCTGACCCTGCTGAAAGCTCT |
| | | CGTGCGGCAGCAGCTGCCTGAGAAGTACAAAGAGATTTTCTTCGA |
| | | CCAGAGCAAGAACGGCTACGCCGGCTACATCGATGGCGGAGCCA |
| | | GCCAGGAAGAGTTCTACAAGTTCATCAAGCCCATCCTGGAAAAGA |
| | | TGGACGGCACCGAGGAACTGCTCGTGAAGCTGAACAGAGAGGAC |
| | | CTGCTGCGGAAGCAGCGGACCTTCGACAACGGCAGCATCCCCCAC |
| | | CAGATCCACCTGGGAGAGCTGCACGCCATTCTGCGGCGGCAGGAA |
| | | GATTTTTACCCATTCCTGAAGGACAACCGGGAAAAGATCGAGAAG |
| | | ATCCTGACCTTCCGCATCCCCTACTACGTGGGCCCTCTGGCCAGGG |
| | | GAAACAGCAGATTCGCCTGGATGACCAGAAAGAGCGAGGAAACC |
| | | ATCACCCCCTGGAACTTCGAGGAAGTGGTGGACAAGGGCGCCAGC |
| | | GCCCAGAGCTTCATCGAGCGGATGACCAACTTCGATAAGAACCTG |
| | | CCCAACGAGAAGGTGCTGCCCAAGCACAGCCTGCTGTACGAGTAC |
| | | TTCACCGTGTACAACGAGCTGACCAAAGTGAAATACGTGACCGAG |
| | | GGAATGAGAAAGCCCGCCTTCCTGAGCGGCGAGCAGAAAAAAGC |
| | | CATCGTGGACCTGCTGTTCAAGACCAACCGGAAAGTGACCGTGAA |
| | | GCAGCTGAAAGAGGACTACTTCAAGAAAATCGAGTGCTTCGACTC |
| | | CGTGGAAATCTCCGGCGTGGAAGATCGGTTCAACGCCTCCCTGGG |
| | | CACATACCACGATCTGCTGAAAATTATCAAGGACAAGGACTTCCTG |
| | | GACAATGAGGAAAACGAGGACATTCTGGAAGATATCGTGCTGACC |
| | | CTGACACTGTTTGAGGACAGAGAGATGATCGAGGAACGGCTGAA |
| | | AACCTATGCCCACCTGTTCGACGACAAAGTGATGAAGCAGCTGAA |
| | | GCGGCGGAGATACACCGGCTGGGGCAGGCTGAGCCGGAAGCTGA |
| | | TCAACGGCATCCGGGACAAGCAGTCCGGCAAGACAATCCTGGATT |
| | | TCCTGAAGTCCGACGGCTTCGCCAACAGAAACTTCATGCAGCTGAT |
| | | CCACGACGACAGCCTGACCTTTAAAGAGGACATCCAGAAAGCCCA |
| | | GGTGTCCGGCCAGGGCGATAGCCTGCACGAGCACATTGCCAATCT |
| | | GGCCGGCAGCCCCGCCATTAAGAAGGGCATCCTGCAGACAGTGAA |
| | | GGTGGTGGACGAGCTCGTGAAAGTGATGGGCCGGCACAAGCCCG |
| | | AGAACATCGTGATCGAAATGGCCAGAGAGAACCAGACCACCCAGA |
| | | AGGGACAGAAGAACAGCCGCGAGAGAATGAAGCGGATCGAAGA |
| | | GGGCATCAAAGAGCTGGGCAGCCAGATCCTGAAAGAACACCCCGT |
| | | GGAAAACACCCAGCTGCAGAACGAGAAGCTGTACCTGTACTACCT |
| | | GCAGAATGGGCGGGATATGTACGTGGACCAGGAACTGGACATCA |
| | | ACCGGCTGTCCGACTACGATGTGGACGCTATCGTGCCTCAGAGCTT |
| | | TCTGAAGGACGACTCCATCGATAACAAAGTGCTGACTCGGAGCGA |
| | | CAAGAACCGGGGCAAGAGCGACAACGTGCCCTCCGAAGAGGTCG |
| | | TGAAGAAGATGAAGAACTACTGGCGCCAGCTGCTGAATGCCAAGC |
| | | TGATTACCCAGAGGAAGTTCGACAATCTGACCAAGGCCGAGAGAG |
| | | GCGGCCTGAGCGAACTGGATAAGGCCGGCTTCATCAAGAGACAG |
| | | CTGGTGGAAACCCGGCAGATCACAAAGCACGTGGCACAGATCCTG |
| | | GACTCCCGGATGAACACTAAGTACGACGAGAACGACAAACTGATC |
| | | CGGGAAGTGAAAGTGATCACCCTGAAGTCCAAGCTGGTGTCCGAT |
| | | TTCCGGAAGGATTTCCAGTTTTACAAAGTGCGCGAGATCAACAACT |
| | | ACCACCACGCCCACGACGCCTACCTGAACGCCGTCGTGGGAACCG |
| | | CCCTGATCAAAAAGTACCCTAAGCTGGAAAGCGAGTTCGTGTACG |
| | | GCGACTACAAGGTGTACGACGTGCGGAAGATGATCGCCAAGAGC |
| | | GAGCAGGAAATCGGCAAGGCTACCGCCAAGTACTTCTTCTACAGC |
| | | AACATCATGAACTTTTTCAAGACCGAGATTACCCTGGCCAACGGCG |
| | | AGATCCGGAAGCGGCCTCTGATCGAGACAAACGGCGAAACAGGC |
| | | GAGATCGTGTGGGATAAGGGCCGGGACTTTGCCACCGTGCGGAA |
| | | AGTGCTGTCTATGCCCCAAGTGAATATCGTGAAAAAGACCGAGGT |
| | | GCAGACAGGCGGCTTCAGCAAAGAGTCTATCCTGCCCAAGAGGAA |
| | | CAGCGACAAGCTGATCGCCAGAAAGAAGGACTGGGACCCTAAGA |
| | | AGTACGGCGGCTTCGACAGCCCCACCGTGGCCTATTCTGTGCTGGT |
| | | GGTGGCCAAAGTGGAAAAGGGCAAGTCCAAGAAACTGAAGAGTG |
| | | TGAAAGAGCTGCTGGGGATCACCATCATGGAAAGAAGCAGCTTCG |
| | | AGAAGAATCCCATCGACTTTCTGGAAGCCAAGGGCTACAAAGAAG |
| | | TGAAAAAGGACCTGATCATCAAGCTGCCTAAGTACTCCCTGTTCGA |
| | | GCTGGAAAACGGCCGGAAGAGAATGCTGGCCTCTGCCGGCGAAC |
| | | TGCAGAAGGGAAACGAACTGGCCCTGCCCTCCAAATATGTGAACT |
| | | TCCTGTACCTGGCCAGCCACTATGAGAAGCTGAAGGGCTCCCCCG |
| | | AGGATAATGAGCAGAAACAGCTGTTTGTGGAACAGCACAAACACT |
| | | ACCTGGACGAGATCATCGAGCAGATCAGCGAGTTCTCCAAGAGAG |
| | | TGATCCTGGCCGACGCTAATCTGGACAAGGTGCTGAGCGCCTACA |
| | | ACAAGCACAGAGACAAGCCTATCAGAGAGCAGGCCGAGAATATC |
| | | ATCCACCTGTTTACCCTGACCAATCTGGGAGCCCCTGCCGCCTTCA |
| | | AGTACTTTGACACCACCATCGACCGGAAGAGGTACACCAGCACCA |
| | | AAGAGGTGCTGGACGCCACCCTGATCCACCAGAGCATCACCGGCC |
| | | TGTACGAGACACGGATCGACCTGTCTCAGCTGGGAGGCGACAGCG |
| | | GAAGTGAGACCCCAGGTACATCCGAATCAGCAACGCCTGAAAGCA |
| | | CCGGTCGGACACTGGTGACCTTCAAGGATGTATTTGTGGACTTCAC |
| | | CAGGGAGGAGTGGAAGCTGCTGGACACTGCTCAGCAGATCGTGT |
| | | ACAGAAATGTGATGCTGGAGAACTATAAGAACCTGGTTTCCTTGG |
| | | GTTATCAGCTTACTAAGCCAGATGTGATCCTCCGGTTGGAGAAGG |
| | | GAGAAGAGCCCAGCGCTGATTACAAAGATGATGACGATAAAGCCC |
| | | CAAAAAAGAAGAGAAAGGTACCGAAGAAAAAAAGAAAGGTC |
| | Fusion Protein 7 | MPKKKRKVPKKKRKVNHDQEFDPPKVYPPVPAEKRKPIRVLSLFDGIA |
| | Amino Acid | TGLLVLKDLGIQVDRYIASEVCEDSITVGMVRHQGKIMYVGDVRSVT |
| | Sequence | QKHIQEWGPFDLVIGGSPCNDLSIVNPARKGLYEGTGRLFFEFYRLLH |
| | NLS-NLS-3A-3L- | DARPKEGDDRPFFWLFENVVAMGVSDKRDISRFLESNPVMIDAKEVS |
| | dCas9-ZIM-NLS- | AAHRARYFWGNLPGMNRPLASTVNDKLELQECLEHGRIAKFSKVRTI |
| | NLS | TTRSNSIKQGKDQHFPVFMNEKEDILWCTEMERVFGFPVHYTDVSN |
| | | MSRLARQRLLGRSWSVPVIRHLFAPLKEYFACVSSGNSNANSRGPSFS |
| | | SGLVPLSLRGSHMGPMEIYKTVSAWKRQPVRVLSLFRNIDKVLKSLGF |
| | | LESGSGSGGGTLKYVEDVTNVVRRDVEKWGPFDLVYGSTQPLGSSCD |
| | | RCPGWYMFQFHRILQYALPRQESQRPFFWIFMDNLLLTEDDQETTT |
| | | RFLQTEAVTLQDVRGRDYQNAMRVWSNIPGLKSKHAPLTPKEEEYLQ |
| | | AQVRSRSKLDAPKVDLLVKNCLLPLREYFKYFSQNSLPLGGPSSGAPPP |
| | | SGGSPAGSPTSTEEGTSESATPESGPGTSTEPSEGSAPGSPAGSPTSTE |
| | | EGTSTEPSEGSAPGTSTEPSEMDKKYSIGLAIGTNSVGWAVITDEYKVP |
| | | SKKFKVLGNTDRHSIKKNLIGALLFDSGETAEATRLKRTARRRYTRRKN |
| | | RICYLQEIFSNEMAKVDDSFFHRLEESFLVEEDKKHERHPIFGNIVDEV |
| | | AYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMIKR |
| | | YDEHHQDLTLLKALVRQQLPEKYKEIFFDQSKNGYAGYIDGGASQEEF |
| | | YKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEE |
| | | TITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDILED |
| | | IVLTLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEM |
| | | ARENQTTQKGQKNSRERMKRIEEGIKELGSQILKEHPVENTQLQNEKL |
| | | YLYYLQNGRDMYVDQELDINRLSDYDVDAIVPQSFLKDDSIDNKVLTR |
| | | SDKNRGKSDNVPSEEVVKKMKNYWRQLLNAKLITQRKFDNLTKAER |
| | | GGLSELDKAGFIKRQLVETRQITKHVAQILDSRMNTKYDENDKLIREVK |
| | | VITLKSKLVSDFRKDFQFYKVREINNYHHAHDAYLNAVVGTALIKKYPK |
| | | LESEFVYGDYKVYDVRKMIAKSEQEIGKATAKYFFYSNIMNFFKTEITLA |
| | | NGEIRKRPLIETNGETGEIVWDKGRDFATVRKVLSMPQVNIVKKTEVQ |
| | | TGGFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKY |
| | | SLFELENGRKRMLASAGELQKGNELALPSKYVNFLYLASHYEKLKGSPE |
| | | DNEQKQLFVEQHKHYLDEIIEQISEFSKRVILADANLDKVLSAYNKHRD |
| | | KPIREQAENIIHLFTLTNLGAPAAFKYFDTTIDRKRYTSTKEVLDATLIHQ |
| | | SITGLYETRIDLSQLGGDSGSETPGTSESATPESTGMNNSQGRVTFED |
| | | VTVNFTQGEWQRLNPEQRNLYRDVMLENYSNLVSVGQGETTKPDVI |
| | | LRLEQGKEPWLEEEEVLGSGRAEKNGDIGGQIWKPKDVKESLSADYK |
| | | DDDDKAPKKKRKVPKKKRKV |
| | Fusion Protein 7 | ATGCCAAAAAAGAAGAGAAAGGTACCGAAGAAAAAAAGAAAGGT |
| | DNA Sequence | ATACAACCATGACCAGGAATTCGACCCCCCAAAGGTTTACCCACCT |
| | | GTGCCAGCTGAGAAGAGGAAGCCCATCCGCGTGCTGTCTCTCTTT |
| | | GATGGGATTGCTACAGGGCTCCTGGTGCTGAAGGACCTGGGCATC |
| | | CAAGTGGACCGCTACATTGCCTCCGAGGTGTGTGAGGACTCCATC |
| | | ACGGTGGGCATGGTGCGGCACCAGGGAAAGATCATGTACGTCGG |
| | | GGACGTCCGCAGCGTCACACAGAAGCATATCCAGGAGTGGGGCC |
| | | CATTCGACCTGGTGATTGGAGGCAGTCCCTGCAATGACCTCTCCAT |
| | | TGTCAACCCTGCCCGCAAGGGACTTTATGAGGGTACTGGCCGCCTC |
| | | TTCTTTGAGTTCTACCGCCTCCTGCATGATGCGCGGCCCAAGGAGG |
| | | GAGATGATCGCCCCTTCTTCTGGCTCTTTGAGAATGTGGTGGCCAT |
| | | GGGCGTTAGTGACAAGAGGGACATCTCGCGATTTCTTGAGTCTAA |
| | | CCCCGTGATGATTGACGCCAAAGAAGTGTCTGCTGCACACAGGGC |
| | | CCGTTACTTCTGGGGTAACCTTCCTGGCATGAACAGGCCTTTGGCA |
| | | TCCACTGTGAATGATAAGCTGGAGCTGCAAGAGTGTCTGGAGCAC |
| | | GGCAGAATAGCCAAGTTCAGCAAAGTGAGGACCATTACCACCAGG |
| | | TCAAACTCTATAAAGCAGGGCAAAGACCAGCATTTCCCCGTCTTCA |
| | | TGAACGAGAAGGAGGACATCCTGTGGTGCACTGAAATGGAAAGG |
| | | GTGTTTGGCTTCCCCGTCCACTACACAGACGTCTCCAACATGAGCC |
| | | GCTTGGCGAGGCAGAGACTGCTGGGCCGATCGTGGAGCGTGCCG |
| | | GTCATCCGCCACCTCTTCGCTCCGCTGAAGGAATATTTTGCTTGTGT |
| | | GTCTAGCGGCAATAGTAACGCTAACAGCCGCGGGCCGAGCTTCAG |
| | | CAGCGGCCTGGTGCCGTTAAGCTTGCGCGGCAGCCATATGGGCCC |
| | | TATGGAGATATACAAGACAGTGTCTGCATGGAAGAGACAGCCAGT |
| | | GCGGGTACTGAGCCTCTTCAGAAACATCGACAAGGTACTAAAGAG |
| | | TTTGGGCTTCTTGGAAAGCGGTTCTGGTTCTGGGGGAGGAACGCT |
| | | GAAGTACGTGGAAGATGTCACAAATGTCGTGAGGAGAGACGTGG |
| | | AGAAATGGGGCCCCTTTGACCTGGTGTACGGCTCGACGCAGCCCC |
| | | TAGGCAGCTCTTGTGATCGCTGTCCCGGCTGGTACATGTTCCAGTT |
| | | CCACCGGATCCTGCAGTATGCGCTGCCTCGCCAGGAGAGTCAGCG |
| | | GCCCTTCTTCTGGATATTCATGGACAATCTGCTGCTGACTGAGGAT |
| | | GACCAAGAGACAACTACCCGCTTCCTTCAGACAGAGGCTGTGACC |
| | | CTCCAGGATGTCCGTGGCAGAGACTACCAGAATGCTATGCGGGTG |
| | | TGGAGCAACATTCCAGGGCTGAAGAGCAAGCATGCGCCCCTGACC |
| | | CCAAAGGAAGAAGAGTATCTGCAAGCCCAAGTCAGAAGCAGGAG |
| | | CAAGCTGGACGCCCCGAAAGTTGACCTCCTGGTGAAGAACTGCCT |
| | | TCTCCCGCTGAGAGAGTACTTCAAGTATTTTTCTCAAAACTCACTTC |
| | | CTCTTGGAGGGCCGAGCTCTGGCGCACCCCCACCAAGTGGAGGGT |
| | | CTCCTGCCGGGTCCCCAACATCTACTGAAGAAGGCACCAGCGAAT |
| | | CCGCAACGCCCGAGTCAGGCCCTGGTACCTCCACAGAACCATCTG |
| | | AAGGTAGTGCGCCTGGTTCCCCAGCTGGAAGCCCTACTTCCACCGA |
| | | AGAAGGCACGTCAACCGAACCAAGTGAAGGATCTGCCCCTGGGAC |
| | | CAGCACTGAACCATCTGAGATGGACAAGAAGTACAGCATCGGCCT |
| | | GGCCATCGGCACCAACTCTGTGGGCTGGGCCGTGATCACCGACGA |
| | | GTACAAGGTGCCCAGCAAGAAATTCAAGGTGCTGGGCAACACCGA |
| | | CCGGCACAGCATCAAGAAGAACCTGATCGGCGCCCTGCTGTTCGA |
| | | CAGCGGAGAAACAGCCGAGGCCACCCGGCTGAAGAGAACCGCCA |
| | | GAAGAAGATACACCAGACGGAAGAACCGGATCTGCTATCTGCAAG |
| | | AGATCTTCAGCAACGAGATGGCCAAGGTGGACGACAGCTTCTTCC |
| | | ACAGACTGGAAGAGTCCTTCCTGGTGGAAGAGGATAAGAAGCAC |
| | | GAGCGGCACCCCATCTTCGGCAACATCGTGGACGAGGTGGCCTAC |
| | | CACGAGAAGTACCCCACCATCTACCACCTGAGAAAGAAACTGGTG |
| | | GACAGCACCGACAAGGCCGACCTGCGGCTGATCTATCTGGCCCTG |
| | | GCCCACATGATCAAGTTCCGGGGCCACTTCCTGATCGAGGGCGAC |
| | | CTGAACCCCGACAACAGCGACGTGGACAAGCTGTTCATCCAGCTG |
| | | GTGCAGACCTACAACCAGCTGTTCGAGGAAAACCCCATCAACGCC |
| | | AGCGGCGTGGACGCCAAGGCCATCCTGTCTGCCAGACTGAGCAAG |
| | | AGCAGACGGCTGGAAAATCTGATCGCCCAGCTGCCCGGCGAGAA |
| | | GAAGAATGGCCTGTTCGGCAACCTGATTGCCCTGAGCCTGGGCCT |
| | | GACCCCCAACTTCAAGAGCAACTTCGACCTGGCCGAGGATGCCAA |
| | | ACTGCAGCTGAGCAAGGACACCTACGACGACGACCTGGACAACCT |
| | | GCTGGCCCAGATCGGCGACCAGTACGCCGACCTGTTTCTGGCCGC |
| | | CAAGAACCTGTCCGACGCCATCCTGCTGAGCGACATCCTGAGAGT |
| | | GAACACCGAGATCACCAAGGCCCCCCTGAGCGCCTCTATGATCAA |
| | | GAGATACGACGAGCACCACCAGGACCTGACCCTGCTGAAAGCTCT |
| | | CGTGCGGCAGCAGCTGCCTGAGAAGTACAAAGAGATTTTCTTCGA |
| | | CCAGAGCAAGAACGGCTACGCCGGCTACATCGATGGCGGAGCCA |
| | | GCCAGGAAGAGTTCTACAAGTTCATCAAGCCCATCCTGGAAAAGA |
| | | TGGACGGCACCGAGGAACTGCTCGTGAAGCTGAACAGAGAGGAC |
| | | CTGCTGCGGAAGCAGCGGACCTTCGACAACGGCAGCATCCCCCAC |
| | | CAGATCCACCTGGGAGAGCTGCACGCCATTCTGCGGCGGCAGGAA |
| | | GATTTTTACCCATTCCTGAAGGACAACCGGGAAAAGATCGAGAAG |
| | | ATCCTGACCTTCCGCATCCCCTACTACGTGGGCCCTCTGGCCAGGG |
| | | GAAACAGCAGATTCGCCTGGATGACCAGAAAGAGCGAGGAAACC |
| | | ATCACCCCCTGGAACTTCGAGGAAGTGGTGGACAAGGGCGCCAGC |
| | | GCCCAGAGCTTCATCGAGCGGATGACCAACTTCGATAAGAACCTG |
| | | CCCAACGAGAAGGTGCTGCCCAAGCACAGCCTGCTGTACGAGTAC |
| | | TTCACCGTGTACAACGAGCTGACCAAAGTGAAATACGTGACCGAG |
| | | GGAATGAGAAAGCCCGCCTTCCTGAGCGGCGAGCAGAAAAAAGC |
| | | CATCGTGGACCTGCTGTTCAAGACCAACCGGAAAGTGACCGTGAA |
| | | GCAGCTGAAAGAGGACTACTTCAAGAAAATCGAGTGCTTCGACTC |
| | | CGTGGAAATCTCCGGCGTGGAAGATCGGTTCAACGCCTCCCTGGG |
| | | CACATACCACGATCTGCTGAAAATTATCAAGGACAAGGACTTCCTG |
| | | GACAATGAGGAAAACGAGGACATTCTGGAAGATATCGTGCTGACC |
| | | CTGACACTGTTTGAGGACAGAGAGATGATCGAGGAACGGCTGAA |
| | | AACCTATGCCCACCTGTTCGACGACAAAGTGATGAAGCAGCTGAA |
| | | GCGGCGGAGATACACCGGCTGGGGCAGGCTGAGCCGGAAGCTGA |
| | | TCAACGGCATCCGGGACAAGCAGTCCGGCAAGACAATCCTGGATT |
| | | TCCTGAAGTCCGACGGCTTCGCCAACAGAAACTTCATGCAGCTGAT |
| | | CCACGACGACAGCCTGACCTTTAAAGAGGACATCCAGAAAGCCCA |
| | | GGTGTCCGGCCAGGGCGATAGCCTGCACGAGCACATTGCCAATCT |
| | | GGCCGGCAGCCCCGCCATTAAGAAGGGCATCCTGCAGACAGTGAA |
| | | GGTGGTGGACGAGCTCGTGAAAGTGATGGGCCGGCACAAGCCCG |
| | | AGAACATCGTGATCGAAATGGCCAGAGAGAACCAGACCACCCAGA |
| | | AGGGACAGAAGAACAGCCGCGAGAGAATGAAGCGGATCGAAGA |
| | | GGGCATCAAAGAGCTGGGCAGCCAGATCCTGAAAGAACACCCCGT |
| | | GGAAAACACCCAGCTGCAGAACGAGAAGCTGTACCTGTACTACCT |
| | | GCAGAATGGGCGGGATATGTACGTGGACCAGGAACTGGACATCA |
| | | ACCGGCTGTCCGACTACGATGTGGACGCTATCGTGCCTCAGAGCTT |
| | | TCTGAAGGACGACTCCATCGATAACAAAGTGCTGACTCGGAGCGA |
| | | CAAGAACCGGGGCAAGAGCGACAACGTGCCCTCCGAAGAGGTCG |
| | | TGAAGAAGATGAAGAACTACTGGCGCCAGCTGCTGAATGCCAAGC |
| | | TGATTACCCAGAGGAAGTTCGACAATCTGACCAAGGCCGAGAGAG |
| | | GCGGCCTGAGCGAACTGGATAAGGCCGGCTTCATCAAGAGACAG |
| | | CTGGTGGAAACCCGGCAGATCACAAAGCACGTGGCACAGATCCTG |
| | | GACTCCCGGATGAACACTAAGTACGACGAGAACGACAAACTGATC |
| | | CGGGAAGTGAAAGTGATCACCCTGAAGTCCAAGCTGGTGTCCGAT |
| | | TTCCGGAAGGATTTCCAGTTTTACAAAGTGCGCGAGATCAACAACT |
| | | ACCACCACGCCCACGACGCCTACCTGAACGCCGTCGTGGGAACCG |
| | | CCCTGATCAAAAAGTACCCTAAGCTGGAAAGCGAGTTCGTGTACG |
| | | GCGACTACAAGGTGTACGACGTGCGGAAGATGATCGCCAAGAGC |
| | | GAGCAGGAAATCGGCAAGGCTACCGCCAAGTACTTCTTCTACAGC |
| | | AACATCATGAACTTTTTCAAGACCGAGATTACCCTGGCCAACGGCG |
| | | AGATCCGGAAGCGGCCTCTGATCGAGACAAACGGCGAAACAGGC |
| | | GAGATCGTGTGGGATAAGGGCCGGGACTTTGCCACCGTGCGGAA |
| | | AGTGCTGTCTATGCCCCAAGTGAATATCGTGAAAAAGACCGAGGT |
| | | GCAGACAGGCGGCTTCAGCAAAGAGTCTATCCTGCCCAAGAGGAA |
| | | CAGCGACAAGCTGATCGCCAGAAAGAAGGACTGGGACCCTAAGA |
| | | AGTACGGCGGCTTCGACAGCCCCACCGTGGCCTATTCTGTGCTGGT |
| | | GGTGGCCAAAGTGGAAAAGGGCAAGTCCAAGAAACTGAAGAGTG |
| | | TGAAAGAGCTGCTGGGGATCACCATCATGGAAAGAAGCAGCTTCG |
| | | AGAAGAATCCCATCGACTTTCTGGAAGCCAAGGGCTACAAAGAAG |
| | | TGAAAAAGGACCTGATCATCAAGCTGCCTAAGTACTCCCTGTTCGA |
| | | GCTGGAAAACGGCCGGAAGAGAATGCTGGCCTCTGCCGGCGAAC |
| | | TGCAGAAGGGAAACGAACTGGCCCTGCCCTCCAAATATGTGAACT |
| | | TCCTGTACCTGGCCAGCCACTATGAGAAGCTGAAGGGCTCCCCCG |
| | | AGGATAATGAGCAGAAACAGCTGTTTGTGGAACAGCACAAACACT |
| | | ACCTGGACGAGATCATCGAGCAGATCAGCGAGTTCTCCAAGAGAG |
| | | TGATCCTGGCCGACGCTAATCTGGACAAGGTGCTGAGCGCCTACA |
| | | ACAAGCACAGAGACAAGCCTATCAGAGAGCAGGCCGAGAATATC |
| | | ATCCACCTGTTTACCCTGACCAATCTGGGAGCCCCTGCCGCCTTCA |
| | | AGTACTTTGACACCACCATCGACCGGAAGAGGTACACCAGCACCA |
| | | AAGAGGTGCTGGACGCCACCCTGATCCACCAGAGCATCACCGGCC |
| | | TGTACGAGACACGGATCGACCTGTCTCAGCTGGGAGGCGACAGCG |
| | | GAAGTGAGACCCCAGGTACATCCGAATCAGCAACGCCTGAAAGCA |
| | | CCGGTATGAACAATTCACAGGGGAGAGTGACATTCGAAGACGTGA |
| | | CCGTGAACTTCACCCAGGGAGAATGGCAGCGCTTGAACCCAGAAC |
| | | AAAGGAACCTCTATCGGGACGTGATGCTGGAAAACTACTCAAATT |
| | | TGGTGAGCGTTGGGCAGGGTGAGACCACTAAGCCTGACGTGATCC |
| | | TGAGATTGGAACAGGGCAAGGAGCCTTGGCTCGAGGAAGAGGAA |
| | | GTCCTGGGCTCAGGGAGGGCCGAGAAAAACGGTGATATAGGAGG |
| | | CCAGATATGGAAGCCTAAGGACGTCAAGGAGAGCCTGAGCGCTG |
| | | ATTACAAAGATGATGACGATAAAGCCCCAAAAAAGAAGAGAAAG |
| | | GTACCGAAGAAAAAAAGAAAGGTC |
| | Fusion Protein 8 | MPKKKRKVPKKKRKVNHDQEFDPPKVYPPVPAEKRKPIRVLSLFDGIA |
| | Amino Acid | TGLLVLKDLGIQVDRYIASEVCEDSITVGMVRHQGKIMYVGDVRSVT |
| | Sequence | QKHIQEWGPFDLVIGGSPCNDLSIVNPARKGLYEGTGRLFFEFYRLLH |
| | NLS-NLS-3A-3L- | DARPKEGDDRPFFWLFENVVAMGVSDKRDISRFLESNPVMIDAKEVS |
| | dCas9-ZFP-NLS- | AAHRARYFWGNLPGMNRPLASTVNDKLELQECLEHGRIAKFSKVRTI |
| | NLS | TTRSNSIKQGKDQHFPVFMNEKEDILWCTEMERVFGFPVHYTDVSN |
| | | MSRLARQRLLGRSWSVPVIRHLFAPLKEYFACVSSGNSNANSRGPSFS |
| | | SGLVPLSLRGSHMGPMEIYKTVSAWKRQPVRVLSLFRNIDKVLKSLGF |
| | | LESGSGSGGGTLKYVEDVTNVVRRDVEKWGPFDLVYGSTQPLGSSCD |
| | | RCPGWYMFQFHRILQYALPRQESQRPFFWIFMDNLLLTEDDQETTT |
| | | RFLQTEAVTLQDVRGRDYQNAMRVWSNIPGLKSKHAPLTPKEEEYLQ |
| | | AQVRSRSKLDAPKVDLLVKNCLLPLREYFKYFSQNSLPLGGPSSGAPPP |
| | | SGGSPAGSPTSTEEGTSESATPESGPGTSTEPSEGSAPGSPAGSPTSTE |
| | | EGTSTEPSEGSAPGTSTEPSEMDKKYSIGLAIGTNSVGWAVITDEYKVP |
| | | SKKFKVLGNTDRHSIKKNLIGALLFDSGETAEATRLKRTARRRYTRRKN |
| | | RICYLQEIFSNEMAKVDDSFFHRLEESFLVEEDKKHERHPIFGNIVDEV |
| | | AYHEKYPTIYHLRKKLVDSTDKADLRLIYLALAHMIKFRGHFLIEGDLNP |
| | | DNSDVDKLFIQLVQTYNQLFEENPINASGVDAKAILSARLSKSRRLENLI |
| | | AQLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMIKR |
| | | YDEHHQDLTLLKALVRQQLPEKYKEIFFDQSKNGYAGYIDGGASQEEF |
| | | YKFIKPILEKMDGTEELLVKLNREDLLRKQRTFDNGSIPHQIHLGELHAI |
| | | LRRQEDFYPFLKDNREKIEKILTFRIPYYVGPLARGNSRFAWMTRKSEE |
| | | TITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKED |
| | | YFKKIECFDSVEISGVEDRFNASLGTYHDLLKIIKDKDFLDNEENEDILED |
| | | IVLTLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEM |
| | | ARENQTTQKGQKNSRERMKRIEEGIKELGSQILKEHPVENTQLQNEKL |
| | | YLYYLQNGRDMYVDQELDINRLSDYDVDAIVPQSFLKDDSIDNKVLTR |
| | | SDKNRGKSDNVPSEEVVKKMKNYWRQLLNAKLITQRKFDNLTKAER |
| | | GGLSELDKAGFIKRQLVETRQITKHVAQILDSRMNTKYDENDKLIREVK |
| | | VITLKSKLVSDFRKDFQFYKVREINNYHHAHDAYLNAVVGTALIKKYPK |
| | | LESEFVYGDYKVYDVRKMIAKSEQEIGKATAKYFFYSNIMNFFKTEITLA |
| | | NGEIRKRPLIETNGETGEIVWDKGRDFATVRKVLSMPQVNIVKKTEVQ |
| | | TGGFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIKLPKY |
| | | SLFELENGRKRMLASAGELQKGNELALPSKYVNFLYLASHYEKLKGSPE |
| | | DNEQKQLFVEQHKHYLDEIIEQISEFSKRVILADANLDKVLSAYNKHRD |
| | | KPIREQAENIIHLFTLTNLGAPAAFKYFDTTIDRKRYTSTKEVLDATLIHQ |
| | | SITGLYETRIDLSQLGGDSGSETPGTSESATPESTGNKKLEAVGTGIEPK |
| | | AMSQGLVTFGDVAVDFSQEEWEWLNPIQRNLYRKVMLENYRNLAS |
| | | LGLCVSKPDVISSLEQGKEPWSADYKDDDDKAPKKKRKVPKKKRKV |
| | Fusion Protein 8 | ATGCCAAAAAAGAAGAGAAAGGTACCGAAGAAAAAAAGAAAGGT |
| | DNA Sequence | ATACAACCATGACCAGGAATTCGACCCCCCAAAGGTTTACCCACCT |
| | | GTGCCAGCTGAGAAGAGGAAGCCCATCCGCGTGCTGTCTCTCTTT |
| | | GATGGGATTGCTACAGGGCTCCTGGTGCTGAAGGACCTGGGCATC |
| | | CAAGTGGACCGCTACATTGCCTCCGAGGTGTGTGAGGACTCCATC |
| | | ACGGTGGGCATGGTGCGGCACCAGGGAAAGATCATGTACGTCGG |
| | | GGACGTCCGCAGCGTCACACAGAAGCATATCCAGGAGTGGGGCC |
| | | CATTCGACCTGGTGATTGGAGGCAGTCCCTGCAATGACCTCTCCAT |
| | | TGTCAACCCTGCCCGCAAGGGACTTTATGAGGGTACTGGCCGCCTC |
| | | TTCTTTGAGTTCTACCGCCTCCTGCATGATGCGCGGCCCAAGGAGG |
| | | GAGATGATCGCCCCTTCTTCTGGCTCTTTGAGAATGTGGTGGCCAT |
| | | GGGCGTTAGTGACAAGAGGGACATCTCGCGATTTCTTGAGTCTAA |
| | | CCCCGTGATGATTGACGCCAAAGAAGTGTCTGCTGCACACAGGGC |
| | | CCGTTACTTCTGGGGTAACCTTCCTGGCATGAACAGGCCTTTGGCA |
| | | TCCACTGTGAATGATAAGCTGGAGCTGCAAGAGTGTCTGGAGCAC |
| | | GGCAGAATAGCCAAGTTCAGCAAAGTGAGGACCATTACCACCAGG |
| | | TCAAACTCTATAAAGCAGGGCAAAGACCAGCATTTCCCCGTCTTCA |
| | | TGAACGAGAAGGAGGACATCCTGTGGTGCACTGAAATGGAAAGG |
| | | GTGTTTGGCTTCCCCGTCCACTACACAGACGTCTCCAACATGAGCC |
| | | GCTTGGCGAGGCAGAGACTGCTGGGCCGATCGTGGAGCGTGCCG |
| | | GTCATCCGCCACCTCTTCGCTCCGCTGAAGGAATATTTTGCTTGTGT |
| | | GTCTAGCGGCAATAGTAACGCTAACAGCCGCGGGCCGAGCTTCAG |
| | | CAGCGGCCTGGTGCCGTTAAGCTTGCGCGGCAGCCATATGGGCCC |
| | | TATGGAGATATACAAGACAGTGTCTGCATGGAAGAGACAGCCAGT |
| | | GCGGGTACTGAGCCTCTTCAGAAACATCGACAAGGTACTAAAGAG |
| | | TTTGGGCTTCTTGGAAAGCGGTTCTGGTTCTGGGGGAGGAACGCT |
| | | GAAGTACGTGGAAGATGTCACAAATGTCGTGAGGAGAGACGTGG |
| | | AGAAATGGGGCCCCTTTGACCTGGTGTACGGCTCGACGCAGCCCC |
| | | TAGGCAGCTCTTGTGATCGCTGTCCCGGCTGGTACATGTTCCAGTT |
| | | CCACCGGATCCTGCAGTATGCGCTGCCTCGCCAGGAGAGTCAGCG |
| | | GCCCTTCTTCTGGATATTCATGGACAATCTGCTGCTGACTGAGGAT |
| | | GACCAAGAGACAACTACCCGCTTCCTTCAGACAGAGGCTGTGACC |
| | | CTCCAGGATGTCCGTGGCAGAGACTACCAGAATGCTATGCGGGTG |
| | | TGGAGCAACATTCCAGGGCTGAAGAGCAAGCATGCGCCCCTGACC |
| | | CCAAAGGAAGAAGAGTATCTGCAAGCCCAAGTCAGAAGCAGGAG |
| | | CAAGCTGGACGCCCCGAAAGTTGACCTCCTGGTGAAGAACTGCCT |
| | | TCTCCCGCTGAGAGAGTACTTCAAGTATTTTTCTCAAAACTCACTTC |
| | | CTCTTGGAGGGCCGAGCTCTGGCGCACCCCCACCAAGTGGAGGGT |
| | | CTCCTGCCGGGTCCCCAACATCTACTGAAGAAGGCACCAGCGAAT |
| | | CCGCAACGCCCGAGTCAGGCCCTGGTACCTCCACAGAACCATCTG |
| | | AAGGTAGTGCGCCTGGTTCCCCAGCTGGAAGCCCTACTTCCACCGA |
| | | AGAAGGCACGTCAACCGAACCAAGTGAAGGATCTGCCCCTGGGAC |
| | | CAGCACTGAACCATCTGAGATGGACAAGAAGTACAGCATCGGCCT |
| | | GGCCATCGGCACCAACTCTGTGGGCTGGGCCGTGATCACCGACGA |
| | | GTACAAGGTGCCCAGCAAGAAATTCAAGGTGCTGGGCAACACCGA |
| | | CCGGCACAGCATCAAGAAGAACCTGATCGGCGCCCTGCTGTTCGA |
| | | CAGCGGAGAAACAGCCGAGGCCACCCGGCTGAAGAGAACCGCCA |
| | | GAAGAAGATACACCAGACGGAAGAACCGGATCTGCTATCTGCAAG |
| | | AGATCTTCAGCAACGAGATGGCCAAGGTGGACGACAGCTTCTTCC |
| | | ACAGACTGGAAGAGTCCTTCCTGGTGGAAGAGGATAAGAAGCAC |
| | | GAGCGGCACCCCATCTTCGGCAACATCGTGGACGAGGTGGCCTAC |
| | | CACGAGAAGTACCCCACCATCTACCACCTGAGAAAGAAACTGGTG |
| | | GACAGCACCGACAAGGCCGACCTGCGGCTGATCTATCTGGCCCTG |
| | | GCCCACATGATCAAGTTCCGGGGCCACTTCCTGATCGAGGGCGAC |
| | | CTGAACCCCGACAACAGCGACGTGGACAAGCTGTTCATCCAGCTG |
| | | GTGCAGACCTACAACCAGCTGTTCGAGGAAAACCCCATCAACGCC |
| | | AGCGGCGTGGACGCCAAGGCCATCCTGTCTGCCAGACTGAGCAAG |
| | | AGCAGACGGCTGGAAAATCTGATCGCCCAGCTGCCCGGCGAGAA |
| | | GAAGAATGGCCTGTTCGGCAACCTGATTGCCCTGAGCCTGGGCCT |
| | | GACCCCCAACTTCAAGAGCAACTTCGACCTGGCCGAGGATGCCAA |
| | | ACTGCAGCTGAGCAAGGACACCTACGACGACGACCTGGACAACCT |
| | | GCTGGCCCAGATCGGCGACCAGTACGCCGACCTGTTTCTGGCCGC |
| | | CAAGAACCTGTCCGACGCCATCCTGCTGAGCGACATCCTGAGAGT |
| | | GAACACCGAGATCACCAAGGCCCCCCTGAGCGCCTCTATGATCAA |
| | | GAGATACGACGAGCACCACCAGGACCTGACCCTGCTGAAAGCTCT |
| | | CGTGCGGCAGCAGCTGCCTGAGAAGTACAAAGAGATTTTCTTCGA |
| | | CCAGAGCAAGAACGGCTACGCCGGCTACATCGATGGCGGAGCCA |
| | | GCCAGGAAGAGTTCTACAAGTTCATCAAGCCCATCCTGGAAAAGA |
| | | TGGACGGCACCGAGGAACTGCTCGTGAAGCTGAACAGAGAGGAC |
| | | CTGCTGCGGAAGCAGCGGACCTTCGACAACGGCAGCATCCCCCAC |
| | | CAGATCCACCTGGGAGAGCTGCACGCCATTCTGCGGCGGCAGGAA |
| | | GATTTTTACCCATTCCTGAAGGACAACCGGGAAAAGATCGAGAAG |
| | | ATCCTGACCTTCCGCATCCCCTACTACGTGGGCCCTCTGGCCAGGG |
| | | GAAACAGCAGATTCGCCTGGATGACCAGAAAGAGCGAGGAAACC |
| | | ATCACCCCCTGGAACTTCGAGGAAGTGGTGGACAAGGGCGCCAGC |
| | | GCCCAGAGCTTCATCGAGCGGATGACCAACTTCGATAAGAACCTG |
| | | CCCAACGAGAAGGTGCTGCCCAAGCACAGCCTGCTGTACGAGTAC |
| | | TTCACCGTGTACAACGAGCTGACCAAAGTGAAATACGTGACCGAG |
| | | GGAATGAGAAAGCCCGCCTTCCTGAGCGGCGAGCAGAAAAAAGC |
| | | CATCGTGGACCTGCTGTTCAAGACCAACCGGAAAGTGACCGTGAA |
| | | GCAGCTGAAAGAGGACTACTTCAAGAAAATCGAGTGCTTCGACTC |
| | | CGTGGAAATCTCCGGCGTGGAAGATCGGTTCAACGCCTCCCTGGG |
| | | CACATACCACGATCTGCTGAAAATTATCAAGGACAAGGACTTCCTG |
| | | GACAATGAGGAAAACGAGGACATTCTGGAAGATATCGTGCTGACC |
| | | CTGACACTGTTTGAGGACAGAGAGATGATCGAGGAACGGCTGAA |
| | | AACCTATGCCCACCTGTTCGACGACAAAGTGATGAAGCAGCTGAA |
| | | GCGGCGGAGATACACCGGCTGGGGCAGGCTGAGCCGGAAGCTGA |
| | | TCAACGGCATCCGGGACAAGCAGTCCGGCAAGACAATCCTGGATT |
| | | TCCTGAAGTCCGACGGCTTCGCCAACAGAAACTTCATGCAGCTGAT |
| | | CCACGACGACAGCCTGACCTTTAAAGAGGACATCCAGAAAGCCCA |
| | | GGTGTCCGGCCAGGGCGATAGCCTGCACGAGCACATTGCCAATCT |
| | | GGCCGGCAGCCCCGCCATTAAGAAGGGCATCCTGCAGACAGTGAA |
| | | GGTGGTGGACGAGCTCGTGAAAGTGATGGGCCGGCACAAGCCCG |
| | | AGAACATCGTGATCGAAATGGCCAGAGAGAACCAGACCACCCAGA |
| | | AGGGACAGAAGAACAGCCGCGAGAGAATGAAGCGGATCGAAGA |
| | | GGGCATCAAAGAGCTGGGCAGCCAGATCCTGAAAGAACACCCCGT |
| | | GGAAAACACCCAGCTGCAGAACGAGAAGCTGTACCTGTACTACCT |
| | | GCAGAATGGGCGGGATATGTACGTGGACCAGGAACTGGACATCA |
| | | ACCGGCTGTCCGACTACGATGTGGACGCTATCGTGCCTCAGAGCTT |
| | | TCTGAAGGACGACTCCATCGATAACAAAGTGCTGACTCGGAGCGA |
| | | CAAGAACCGGGGCAAGAGCGACAACGTGCCCTCCGAAGAGGTCG |
| | | TGAAGAAGATGAAGAACTACTGGCGCCAGCTGCTGAATGCCAAGC |
| | | TGATTACCCAGAGGAAGTTCGACAATCTGACCAAGGCCGAGAGAG |
| | | GCGGCCTGAGCGAACTGGATAAGGCCGGCTTCATCAAGAGACAG |
| | | CTGGTGGAAACCCGGCAGATCACAAAGCACGTGGCACAGATCCTG |
| | | GACTCCCGGATGAACACTAAGTACGACGAGAACGACAAACTGATC |
| | | CGGGAAGTGAAAGTGATCACCCTGAAGTCCAAGCTGGTGTCCGAT |
| | | TTCCGGAAGGATTTCCAGTTTTACAAAGTGCGCGAGATCAACAACT |
| | | ACCACCACGCCCACGACGCCTACCTGAACGCCGTCGTGGGAACCG |
| | | CCCTGATCAAAAAGTACCCTAAGCTGGAAAGCGAGTTCGTGTACG |
| | | GCGACTACAAGGTGTACGACGTGCGGAAGATGATCGCCAAGAGC |
| | | GAGCAGGAAATCGGCAAGGCTACCGCCAAGTACTTCTTCTACAGC |
| | | AACATCATGAACTTTTTCAAGACCGAGATTACCCTGGCCAACGGCG |
| | | AGATCCGGAAGCGGCCTCTGATCGAGACAAACGGCGAAACAGGC |
| | | GAGATCGTGTGGGATAAGGGCCGGGACTTTGCCACCGTGCGGAA |
| | | AGTGCTGTCTATGCCCCAAGTGAATATCGTGAAAAAGACCGAGGT |
| | | GCAGACAGGCGGCTTCAGCAAAGAGTCTATCCTGCCCAAGAGGAA |
| | | CAGCGACAAGCTGATCGCCAGAAAGAAGGACTGGGACCCTAAGA |
| | | AGTACGGCGGCTTCGACAGCCCCACCGTGGCCTATTCTGTGCTGGT |
| | | GGTGGCCAAAGTGGAAAAGGGCAAGTCCAAGAAACTGAAGAGTG |
| | | TGAAAGAGCTGCTGGGGATCACCATCATGGAAAGAAGCAGCTTCG |
| | | AGAAGAATCCCATCGACTTTCTGGAAGCCAAGGGCTACAAAGAAG |
| | | TGAAAAAGGACCTGATCATCAAGCTGCCTAAGTACTCCCTGTTCGA |
| | | GCTGGAAAACGGCCGGAAGAGAATGCTGGCCTCTGCCGGCGAAC |
| | | TGCAGAAGGGAAACGAACTGGCCCTGCCCTCCAAATATGTGAACT |
| | | TCCTGTACCTGGCCAGCCACTATGAGAAGCTGAAGGGCTCCCCCG |
| | | AGGATAATGAGCAGAAACAGCTGTTTGTGGAACAGCACAAACACT |
| | | ACCTGGACGAGATCATCGAGCAGATCAGCGAGTTCTCCAAGAGAG |
| | | TGATCCTGGCCGACGCTAATCTGGACAAGGTGCTGAGCGCCTACA |
| | | ACAAGCACAGAGACAAGCCTATCAGAGAGCAGGCCGAGAATATC |
| | | ATCCACCTGTTTACCCTGACCAATCTGGGAGCCCCTGCCGCCTTCA |
| | | AGTACTTTGACACCACCATCGACCGGAAGAGGTACACCAGCACCA |
| | | AAGAGGTGCTGGACGCCACCCTGATCCACCAGAGCATCACCGGCC |
| | | TGTACGAGACACGGATCGACCTGTCTCAGCTGGGAGGCGACAGCG |
| | | GAAGTGAGACCCCAGGTACATCCGAATCAGCAACGCCTGAAAGCA |
| | | CCGGTAACAAAAAGCTTGAGGCCGTCGGAACCGGAATCGAACCAA |
| | | AAGCAATGTCCCAGGGTTTGGTGACATTTGGCGACGTGGCTGTCG |
| | | ATTTTTCCCAGGAAGAGTGGGAGTGGCTCAATCCTATCCAGAGGA |
| | | ACTTGTACCGGAAGGTGATGCTGGAGAATTATAGAAATTTGGCAT |
| | | CACTGGGGTTGTGCGTTAGCAAACCAGATGTTATATCTTCCCTGGA |
| | | ACAGGGAAAGGAGCCCTGGAGCGCTGATTACAAAGATGATGACG |
| | | ATAAAGCCCCAAAAAAGAAGAGAAAGGTACCGAAGAAAAAAAGA |
| | | AAGGTC |
| | Fusion Protein 9 | MPKKKRKVPKKKRKVYNHDQEFDPPKVYPPVPAEKRKPIRVLSLFDGI |
| | Amino Acid | ATGLLVLKDLGIQVDRYIASEVCEDSITVGMVRHQGKIMYVGDVRSVT |
| | Sequence | QKHIQEWGPFDLVIGGSPCNDLSIVNPARKGLYEGTGRLFFEFYRLLH |
| | NLS-NLS-3A-ADD | DARPKEGDDRPFFWLFENVVAMGVSDKRDISRFLESNPVMIDAKEVS |
| | -h3L-dCas9- | AAHRARYFWGNLPGMNRPLASTVNDKLELQECLEHGRIAKFSKVRTI |
| | KOX1KRAB-NLS- | TTRSNSIKQGKDQHFPVFMNEKEDILWCTEMERVFGFPVHYTDVSN |
| | NLS | MSRLARQRLLGRSWSVPVIRHLFAPLKEYFACVSSGNSNANSRGPSFS |
| | | SGLVPLSLRGSHMAAIPALDPEAEPSMDVILVGSSELSSSVSPGTGRDL |
| | | IAYEVKANQRNIEDICICCGSLQVHTQHPLFEGGICAPCKDKFLDALFLY |
| | | DDDGYQSYCSICCSGETLLICGNPDCTRCYCFECVDSLVGPGTSGKVH |
| | | AMSNWVCYLCLPSSRSGLLQRRRKWRSQLKAFYDRESENPLEMFETV |
| | | PVWRRQPVRVLSLFEDIKKELTSLGFLESGSDPGQLKHVVDVTDTVRK |
| | | DVEEWGPFDLVYGATPPLGHTCDRPPSWYLFQFHRLLQYARPKPGSP |
| | | RPFFWMFVDNLVLNKEDLDVASRFLEMEPVTIPDVHGGSLQNAVRV |
| | | WSNIPAIRSRHWALVSEEELSLLAQNKQSSKLAAKWPTKLVKNCFLPL |
| | | REYFKYFSTELTSSPGGPSSGAPPPSGGSPAGSPTSTEEGTSESATPESG |
| | | PGTSTEPSEGSAPGSPAGSPTSTEEGTSTEPSEGSAPGTSTEPSELEDKK |
| | | YSIGLAIGTNSVGWAVITDEYKVPSKKFKVLGNTDRHSIKKNLIGALLFD |
| | | SGETAEATRLKRTARRRYTRRKNRICYLQEIFSNEMAKVDDSFFHRLEE |
| | | SFLVEEDKKHERHPIFGNIVDEVAYHEKYPTIYHLRKKLVDSTDKADLRL |
| | | IYLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTPNF |
| | | KSNFDLAEDAKLQLSKDTYDDDLDNLLAQIGDQYADLFLAAKNLSDAI |
| | | LLSDILRVNTEITKAPLSASMIKRYDEHHQDLTLLKALVRQQLPEKYKEI |
| | | FFDQSKNGYAGYIDGGASQEEFYKFIKPILEKMDGTEELLVKLNREDLL |
| | | RKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMT |
| | | NFDKNLPNEKVLPKHSLLYEYFTVYNELTKVKYVTEGMRKPAFLSGEQ |
| | | KKAIVDLLFKTNRKVTVKQLKEDYFKKIECFDSVEISGVEDRFNASLGTY |
| | | HDLLKIIKDKDFLDNEENEDILEDIVLTLTLFEDREMIEERLKTYAHLFDD |
| | | KVMKQLKRRRYTGWGRLSRKLINGIRDKQSGKTILDFLKSDGFANRNF |
| | | MQLIHDDSLTFKEDIQKAQVSGQGDSLHEHIANLAGSPAIKKGILQTV |
| | | KVVDELVKVMGRHKPENIVIEMARENQTTQKGQKNSRERMKRIEEGI |
| | | KELGSQILKEHPVENTQLQNEKLYLYYLQNGRDMYVDQELDINRLSDY |
| | | DVDAIVPQSFLKDDSIDNKVLTRSDKNRGKSDNVPSEEVVKKMKNY |
| | | WRQLLNAKLITQRKFDNLTKAERGGLSELDKAGFIKRQLVETRQITKH |
| | | VAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKG |
| | | RDFATVRKVLSMPQVNIVKKTEVQTGGFSKESILPKRNSDKLIARKKD |
| | | WDPKKYGGFDSPTVAYSVLVVAKVEKGKSKKLKSVKELLGITIMERSSF |
| | | EKNPIDFLEAKGYKEVKKDLIIKLPKYSLFELENGRKRMLASAGELQKG |
| | | NELALPSKYVNFLYLASHYEKLKGSPEDNEQKQLFVEQHKHYLDEIIEQI |
| | | SEFSKRVILADANLDKVLSAYNKHRDKPIREQAENIIHLFTLTNLGAPAA |
| | | FKYFDTTIDRKRYTSTKEVLDATLIHQSITGLYETRIDLSQLGGDSPKKKR |
| | | KVGVDGSSGSETPGTSESATPESRTLVTFKDVFVDFTREEWKLLDTAQ |
| | | QIVYRNVMLENYKNLVSLGYQLTKPDVILRLEKGEEPSADYKDDDDKA |
| | | PKKKRKVPKKKRKV |
| | Fusion Protein 9 | ATGGGTACCATGCCAAAAAAGAAGAGAAAGGTACCGAAGAAAAA |
| | DNA Sequence | AAGAAAGGTATACAATCACGATCAGGAGTTCGACCCCCCTAAGGT |
| | | GTACCCACCAGTGCCTGCAGAGAAGAGGAAGCCAATCCGGGTGCT |
| | | GAGCCTGTTTGATGGCATCGCCACCGGCCTGCTGGTGCTGAAGGA |
| | | TCTGGGCATCCAGGTGGACCGGTACATCGCCTCCGAGGTGTGCGA |
| | | GGATTCTATCACCGTGGGCATGGTGCGCCACCAGGGCAAGATCAT |
| | | GTATGTGGGCGACGTGCGGTCCGTGACACAGAAGCACATCCAGG |
| | | AGTGGGGCCCATTCGATCTGGTGATCGGCGGCAGCCCCTGTAATG |
| | | ACCTGTCCATCGTGAACCCTGCAAGGAAGGGACTGTACGAGGGAA |
| | | CCGGCCGGCTGTTCTTTGAGTTTTATAGACTGCTGCACGACGCCAG |
| | | GCCTAAGGAGGGCGACGATAGACCATTCTTTTGGCTGTTCGAGAA |
| | | TGTGGTGGCTATGGGCGTGAGCGATAAGAGGGACATCTCCAGGTT |
| | | TCTGGAGTCTAACCCCGTGATGATCGATGCAAAGGAGGTGTCCGC |
| | | CGCACACAGAGCCAGGTATTTCTGGGGCAATCTGCCAGGAATGAA |
| | | CAGGCCACTGGCAAGCACCGTGAATGACAAGCTGGAGCTGCAGG |
| | | AGTGCCTGGAGCACGGAAGGATCGCCAAGTTTTCCAAGGTGCGCA |
| | | CAATCACCACACGGAGCAATTCCATCAAGCAGGGCAAGGATCAGC |
| | | ACTTCCCCGTGTTCATGAACGAGAAGGAGGACATCCTGTGGTGTA |
| | | CCGAGATGGAGAGAGTGTTCGGCTTTCCAGTGCACTACACAGACG |
| | | TGTCTAACATGAGCAGGCTGGCAAGGCAGCGGCTGCTGGGCAGA |
| | | TCTTGGAGCGTGCCCGTGATCAGGCACCTGTTCGCCCCTCTGAAGG |
| | | AGTATTTTGCCTGCGTGAGCAGCGGCAACTCCAATGCCAACAGCC |
| | | GGGGCCCCTCTTTCAGCTCCGGATTGGTGCCTCTGAGCCTGAGGG |
| | | GCTCCCACATGGCAGCAATCCCCGCCCTGGACCCCGAGGCCGAGC |
| | | CTAGCATGGACGTGATCCTGGTGGGCTCTAGCGAGCTGTCCTCTA |
| | | GCGTGTCTCCAGGAACCGGAAGGGATCTGATCGCATACGAGGTGA |
| | | AGGCCAATCAGCGGAACATCGAGGACATCTGTATCTGCTGTGGCA |
| | | GCCTGCAGGTGCACACACAGCACCCACTGTTCGAGGGAGGAATCT |
| | | GCGCACCCTGTAAGGATAAGTTCCTGGACGCCCTGTTTCTGTACGA |
| | | CGATGACGGCTACCAGTCCTATTGCTCTATCTGCTGTTCCGGCGAG |
| | | ACCCTGCTGATCTGCGGCAATCCAGATTGTACAAGGTGCTATTGTT |
| | | TTGAGTGCGTGGACTCTCTGGTGGGACCAGGCACCAGCGGAAAG |
| | | GTGCACGCCATGTCCAACTGGGTGTGCTACCTGTGCCTGCCATCCT |
| | | CTCGCAGCGGACTGCTGCAGCGGAGAAGGAAGTGGAGATCCCAG |
| | | CTGAAGGCCTTCTATGATAGGGAGTCTGAGAACCCCCTGGAGATG |
| | | TTTGAGACCGTGCCAGTGTGGCGCCGGCAGCCCGTGAGGGTGCTG |
| | | AGCCTGTTCGAGGATATCAAGAAGGAGCTGACATCCCTGGGCTTT |
| | | CTGGAGTCCGGCTCTGACCCCGGACAGCTGAAGCACGTGGTGGAT |
| | | GTGACCGACACAGTGCGGAAGGATGTGGAGGAGTGGGGCCCTTT |
| | | CGACCTGGTGTACGGAGCAACCCCTCCACTGGGACACACATGCGA |
| | | CAGACCCCCTTCTTGGTACCTGTTCCAGTTTCACCGCCTGCTGCAGT |
| | | ATGCAAGGCCAAAGCCAGGCAGCCCTAGACCATTCTTTTGGATGTT |
| | | CGTGGATAATCTGGTGCTGAACAAGGAGGATCTGGACGTGGCCA |
| | | GCAGGTTTCTGGAGATGGAGCCAGTGACCATCCCAGACGTGCACG |
| | | GCGGCTCCCTGCAGAATGCCGTGCGCGTGTGGTCTAACATCCCTGC |
| | | CATCAGAAGCAGGCACTGGGCACTGGTGAGCGAGGAGGAGCTGT |
| | | CCCTGCTGGCCCAGAATAAGCAGAGCAGCAAGCTGGCCGCCAAGT |
| | | GGCCTACAAAGCTGGTGAAGAACTGCTTCCTGCCACTGCGGGAGT |
| | | ACTTCAAGTATTTTTCCACCGAGCTGACATCTAGCCTGGGAGGACC |
| | | CTCCTCTGGCGCCCCACCACCTAGCGGCGGCTCCCCTGCCGGCTCT |
| | | CCAACCAGCACAGAGGAGGGCACCAGCGAGTCCGCCACACCAGA |
| | | GTCTGGACCTGGCACCAGCACAGAGCCATCCGAGGGCTCTGCCCC |
| | | AGGCTCTCCTGCAGGCAGCCCTACCTCCACCGAAGAGGGCACCAG |
| | | CACAGAGCCTTCTGAGGGCAGCGCCCCAGGCACCTCTACAGAGCC |
| | | AAGCGAGCTCGAGGACAAGAAGTACAGCATCGGCCTGGCCATCG |
| | | GCACCAACTCTGTGGGCTGGGCCGTGATCACCGACGAGTACAAGG |
| | | TGCCCAGCAAGAAATTCAAGGTGCTGGGCAACACCGACCGGCACA |
| | | GCATCAAGAAGAACCTGATCGGAGCCCTGCTGTTCGACAGCGGCG |
| | | AAACAGCCGAGGCCACCCGGCTGAAGAGAACCGCCAGAAGAAGA |
| | | TACACCAGACGGAAGAACCGGATCTGCTATCTGCAAGAGATCTTC |
| | | AGCAACGAGATGGCCAAGGTGGACGACAGCTTCTTCCACAGACTG |
| | | GAAGAGTCCTTCCTGGTGGAAGAGGATAAGAAGCACGAGCGGCA |
| | | CCCCATCTTCGGCAACATCGTGGACGAGGTGGCCTACCACGAGAA |
| | | GTACCCCACCATCTACCACCTGAGAAAGAAACTGGTGGACAGCAC |
| | | CGACAAGGCCGACCTGCGGCTGATCTATCTGGCCCTGGCCCACAT |
| | | GATCAAGTTCCGGGGCCACTTCCTGATCGAGGGCGACCTGAACCC |
| | | CGACAACAGCGACGTGGACAAGCTGTTCATCCAGCTGGTGCAGAC |
| | | CTACAACCAGCTGTTCGAGGAAAACCCCATCAACGCCAGCGGCGT |
| | | GGACGCCAAGGCCATCCTGTCTGCCAGACTGAGCAAGAGCAGACG |
| | | GCTGGAAAATCTGATCGCCCAGCTGCCCGGCGAGAAGAAGAATG |
| | | GCCTGTTCGGCAACCTGATTGCCCTGAGCCTGGGCCTGACCCCCAA |
| | | CTTCAAGAGCAACTTCGACCTGGCCGAGGATGCCAAACTGCAGCT |
| | | GAGCAAGGACACCTACGACGACGACCTGGACAACCTGCTGGCCCA |
| | | GATCGGCGACCAGTACGCCGACCTGTTTCTGGCCGCCAAGAACCT |
| | | GTCCGACGCCATCCTGCTGAGCGACATCCTGAGAGTGAACACCGA |
| | | GATCACCAAGGCCCCCCTGAGCGCCTCTATGATCAAGAGATACGA |
| | | CGAGCACCACCAGGACCTGACCCTGCTGAAAGCTCTCGTGCGGCA |
| | | GCAGCTGCCTGAGAAGTACAAAGAGATTTTCTTCGACCAGAGCAA |
| | | GAACGGCTACGCCGGCTACATTGACGGCGGAGCCAGCCAGGAAG |
| | | AGTTCTACAAGTTCATCAAGCCCATCCTGGAAAAGATGGACGGCA |
| | | CCGAGGAACTGCTCGTGAAGCTGAACAGAGAGGACCTGCTGCGG |
| | | AAGCAGCGGACCTTCGACAACGGCAGCATCCCCCACCAGATCCAC |
| | | CTGGGAGAGCTGCACGCCATTCTGCGGCGGCAGGAAGATTTTTAC |
| | | CCATTCCTGAAGGACAACCGGGAAAAGATCGAGAAGATCCTGACC |
| | | TTCCGCATCCCCTACTACGTGGGCCCTCTGGCCAGGGGAAACAGC |
| | | AGATTCGCCTGGATGACCAGAAAGAGCGAGGAAACCATCACCCCC |
| | | TGGAACTTCGAGGAAGTGGTGGACAAGGGCGCTTCCGCCCAGAG |
| | | CTTCATCGAGCGGATGACCAACTTCGATAAGAACCTGCCCAACGA |
| | | GAAGGTGCTGCCCAAGCACAGCCTGCTGTACGAGTACTTCACCGT |
| | | GTATAACGAGCTGACCAAAGTGAAATACGTGACCGAGGGAATGA |
| | | GAAAGCCCGCCTTCCTGAGCGGCGAGCAGAAAAAGGCCATCGTG |
| | | GACCTGCTGTTCAAGACCAACCGGAAAGTGACCGTGAAGCAGCTG |
| | | AAAGAGGACTACTTCAAGAAAATCGAGTGCTTCGACTCCGTGGAA |
| | | ATCTCCGGCGTGGAAGATCGGTTCAACGCCTCCCTGGGCACATACC |
| | | ACGATCTGCTGAAAATTATCAAGGACAAGGACTTCCTGGACAATG |
| | | AGGAAAACGAGGACATTCTGGAAGATATCGTGCTGACCCTGACAC |
| | | TGTTTGAGGACAGAGAGATGATCGAGGAACGGCTGAAAACCTAT |
| | | GCCCACCTGTTCGACGACAAAGTGATGAAGCAGCTGAAGCGGCG |
| | | GAGATACACCGGCTGGGGCAGGCTGAGCCGGAAGCTGATCAACG |
| | | GCATCCGGGACAAGCAGTCCGGCAAGACAATCCTGGATTTCCTGA |
| | | AGTCCGACGGCTTCGCCAACAGAAACTTCATGCAGCTGATCCACG |
| | | ACGACAGCCTGACCTTTAAAGAGGACATCCAGAAAGCCCAGGTGT |
| | | CCGGCCAGGGCGATAGCCTGCACGAGCACATTGCCAATCTGGCCG |
| | | GCAGCCCCGCCATTAAGAAGGGCATCCTGCAGACAGTGAAGGTG |
| | | GTGGACGAGCTCGTGAAAGTGATGGGCCGGCACAAGCCCGAGAA |
| | | CATCGTGATCGAAATGGCCAGAGAGAACCAGACCACCCAGAAGG |
| | | GACAGAAGAACAGCCGCGAGAGAATGAAGCGGATCGAAGAGGG |
| | | CATCAAAGAGCTGGGCAGCCAGATCCTGAAAGAACACCCCGTGGA |
| | | AAACACCCAGCTGCAGAACGAGAAGCTGTACCTGTACTACCTGCA |
| | | GAATGGGCGGGATATGTACGTGGACCAGGAACTGGACATCAACC |
| | | GGCTGTCCGACTACGATGTGGACGCCATCGTGCCTCAGAGCTTTCT |
| | | GAAGGACGACTCCATCGACAACAAGGTGCTGACCAGAAGCGACA |
| | | AGAACCGGGGCAAGAGCGACAACGTGCCCTCCGAAGAGGTCGTG |
| | | AAGAAGATGAAGAACTACTGGCGGCAGCTGCTGAACGCCAAGCT |
| | | GATTACCCAGAGAAAGTTCGACAATCTGACCAAGGCCGAGAGAGG |
| | | CGGCCTGAGCGAACTGGATAAGGCCGGCTTCATCAAGAGACAGCT |
| | | GGTGGAAACCCGGCAGATCACAAAGCACGTGGCACAGATCCTGG |
| | | ACTCCCGGATGAACACTAAGTACGACGAGAATGACAAGCTGATCC |
| | | GGGAAGTGAAAGTGATCACCCTGAAGTCCAAGCTGGTGTCCGATT |
| | | TCCGGAAGGATTTCCAGTTTTACAAAGTGCGCGAGATCAACAACTA |
| | | CCACCACGCCCACGACGCCTACCTGAACGCCGTCGTGGGAACCGC |
| | | CCTGATCAAAAAGTACCCTAAGCTGGAAAGCGAGTTCGTGTACGG |
| | | CGACTACAAGGTGTACGACGTGCGGAAGATGATCGCCAAGAGCG |
| | | AGCAGGAAATCGGCAAGGCTACCGCCAAGTACTTCTTCTACAGCA |
| | | ACATCATGAACTTTTTCAAGACCGAGATTACCCTGGCCAACGGCGA |
| | | GATCCGGAAGCGGCCTCTGATCGAGACAAACGGCGAAACCGGGG |
| | | AGATCGTGTGGGATAAGGGCCGGGATTTTGCCACCGTGCGGAAA |
| | | GTGCTGAGCATGCCCCAAGTGAATATCGTGAAAAAGACCGAGGTG |
| | | CAGACAGGCGGCTTCAGCAAAGAGTCTATCCTGCCCAAGAGGAAC |
| | | AGCGATAAGCTGATCGCCAGAAAGAAGGACTGGGACCCTAAGAA |
| | | GTACGGCGGCTTCGACAGCCCCACCGTGGCCTATTCTGTGCTGGTG |
| | | GTGGCCAAAGTGGAAAAGGGCAAGTCCAAGAAACTGAAGAGTGT |
| | | GAAAGAGCTGCTGGGGATCACCATCATGGAAAGAAGCAGCTTCGA |
| | | GAAGAATCCCATCGACTTTCTGGAAGCCAAGGGCTACAAAGAAGT |
| | | GAAAAAGGACCTGATCATCAAGCTGCCTAAGTACTCCCTGTTCGAG |
| | | CTGGAAAACGGCCGGAAGAGAATGCTGGCCTCTGCCGGCGAACT |
| | | GCAGAAGGGAAACGAACTGGCCCTGCCCTCCAAATATGTGAACTT |
| | | CCTGTACCTGGCCAGCCACTATGAGAAGCTGAAGGGCTCCCCCGA |
| | | GGATAATGAGCAGAAACAGCTGTTTGTGGAACAGCACAAGCACTA |
| | | CCTGGACGAGATCATCGAGCAGATCAGCGAGTTCTCCAAGAGAGT |
| | | GATCCTGGCCGACGCTAATCTGGACAAAGTGCTGTCCGCCTACAAC |
| | | AAGCACCGGGATAAGCCCATCAGAGAGCAGGCCGAGAATATCATC |
| | | CACCTGTTTACCCTGACCAATCTGGGAGCCCCTGCCGCCTTCAAGT |
| | | ACTTTGACACCACCATCGACCGGAAGAGGTACACCAGCACCAAAG |
| | | AGGTGCTGGACGCCACCCTGATCCACCAGAGCATCACCGGCCTGT |
| | | ACGAGACACGGATCGACCTGTCTCAGCTGGGAGGCGACAGCCCCA |
| | | AGAAGAAGAGAAAGGTGGGAGTCGACGGATCCAGCGGCTCCGAG |
| | | ACCCCAGGCACATCTGAGAGCGCCACCCCTGAGTCCCGGACCCTG |
| | | GTGACCTTCAAGGATGTATTTGTGGACTTCACCAGGGAGGAGTGG |
| | | AAGCTGCTGGACACTGCTCAGCAGATCGTGTACAGAAATGTGATG |
| | | CTGGAGAACTATAAGAACCTGGTTTCCTTGGGTTATCAGCTTACTA |
| | | AGCCAGATGTGATCCTCCGGTTGGAGAAGGGAGAAGAGCCCAGC |
| | | GCTGATTACAAAGATGATGACGATAAAGCCCCAAAAAAGAAGAGA |
| | | AAGGTACCGAAGAAAAAAAGAAAGGTCTGA |
| | Fusion Protein | MGTMPKKKRKVPKKKRKVYNHDQEFDPPKVYPPVPAEKRKPIRVLSL |
| | 10 Amino Acid | FDGIATGLLVLKDLGIQVDRYIASEVCEDSITVGMVRHQGKIMYVGDV |
| | Sequence | RSVTQKHIQEWGPFDLVIGGSPCNDLSIVNPARKGLYEGTGRLFFEFY |
| | NLS-NLS-3A- | RLLHDARPKEGDDRPFFWLFENVVAMGVSDKRDISRFLESNPVMIDA |
| | ADD-h3L-dCas9- | KEVSAAHRARYFWGNLPGMNRPLASTVNDKLELQECLEHGRIAKFSK |
| | ZFP-28-NLS-NLS | VRTITTRSNSIKQGKDQHFPVFMNEKEDILWCTEMERVFGFPVHYTD |
| | | VSNMSRLARQRLLGRSWSVPVIRHLFAPLKEYFACVSSGNSNANSRG |
| | | PSFSSGLVPLSLRGSHMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSLGGPSSGAPPPSGGSPAGSPTSTEEGTSES |
| | | ATPESGPGTSTEPSEGSAPGSPAGSPTSTEEGTSTEPSEGSAPGTSTEP |
| | | SELEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSSGSETPGTSESATPESTGNKKLEAVGTGIEPKAMSQ |
| | | GLVTFGDVAVDFSQEEWEWLNPIQRNLYRKVMLENYRNLASLGLCV |
| | | SKPDVISSLEQGKEPWSADYKDDDDKAPKKKRKVPKKKRKV |
| | Fusion Protein | ATGGGTACCATGCCAAAAAAGAAGAGAAAGGTACCGAAGAAAAA |
| | 10 DNA | AAGAAAGGTATACAATCACGATCAGGAGTTCGACCCCCCTAAGGT |
| | Sequence | GTACCCACCAGTGCCTGCAGAGAAGAGGAAGCCAATCCGGGTGCT |
| | | GAGCCTGTTTGATGGCATCGCCACCGGCCTGCTGGTGCTGAAGGA |
| | | TCTGGGCATCCAGGTGGACCGGTACATCGCCTCCGAGGTGTGCGA |
| | | GGATTCTATCACCGTGGGCATGGTGCGCCACCAGGGCAAGATCAT |
| | | GTATGTGGGCGACGTGCGGTCCGTGACACAGAAGCACATCCAGG |
| | | AGTGGGGCCCATTCGATCTGGTGATCGGCGGCAGCCCCTGTAATG |
| | | ACCTGTCCATCGTGAACCCTGCAAGGAAGGGACTGTACGAGGGAA |
| | | CCGGCCGGCTGTTCTTTGAGTTTTATAGACTGCTGCACGACGCCAG |
| | | GCCTAAGGAGGGCGACGATAGACCATTCTTTTGGCTGTTCGAGAA |
| | | TGTGGTGGCTATGGGCGTGAGCGATAAGAGGGACATCTCCAGGTT |
| | | TCTGGAGTCTAACCCCGTGATGATCGATGCAAAGGAGGTGTCCGC |
| | | CGCACACAGAGCCAGGTATTTCTGGGGCAATCTGCCAGGAATGAA |
| | | CAGGCCACTGGCAAGCACCGTGAATGACAAGCTGGAGCTGCAGG |
| | | AGTGCCTGGAGCACGGAAGGATCGCCAAGTTTTCCAAGGTGCGCA |
| | | CAATCACCACACGGAGCAATTCCATCAAGCAGGGCAAGGATCAGC |
| | | ACTTCCCCGTGTTCATGAACGAGAAGGAGGACATCCTGTGGTGTA |
| | | CCGAGATGGAGAGAGTGTTCGGCTTTCCAGTGCACTACACAGACG |
| | | TGTCTAACATGAGCAGGCTGGCAAGGCAGCGGCTGCTGGGCAGA |
| | | TCTTGGAGCGTGCCCGTGATCAGGCACCTGTTCGCCCCTCTGAAGG |
| | | AGTATTTTGCCTGCGTGAGCAGCGGCAACTCCAATGCCAACAGCC |
| | | GGGGCCCCTCTTTCAGCTCCGGATTGGTGCCTCTGAGCCTGAGGG |
| | | GCTCCCACATGGCAGCAATCCCCGCCCTGGACCCCGAGGCCGAGC |
| | | CTAGCATGGACGTGATCCTGGTGGGCTCTAGCGAGCTGTCCTCTA |
| | | GCGTGTCTCCAGGAACCGGAAGGGATCTGATCGCATACGAGGTGA |
| | | AGGCCAATCAGCGGAACATCGAGGACATCTGTATCTGCTGTGGCA |
| | | GCCTGCAGGTGCACACACAGCACCCACTGTTCGAGGGAGGAATCT |
| | | GCGCACCCTGTAAGGATAAGTTCCTGGACGCCCTGTTTCTGTACGA |
| | | CGATGACGGCTACCAGTCCTATTGCTCTATCTGCTGTTCCGGCGAG |
| | | ACCCTGCTGATCTGCGGCAATCCAGATTGTACAAGGTGCTATTGTT |
| | | TTGAGTGCGTGGACTCTCTGGTGGGACCAGGCACCAGCGGAAAG |
| | | GTGCACGCCATGTCCAACTGGGTGTGCTACCTGTGCCTGCCATCCT |
| | | CTCGCAGCGGACTGCTGCAGCGGAGAAGGAAGTGGAGATCCCAG |
| | | CTGAAGGCCTTCTATGATAGGGAGTCTGAGAACCCCCTGGAGATG |
| | | TTTGAGACCGTGCCAGTGTGGCGCCGGCAGCCCGTGAGGGTGCTG |
| | | AGCCTGTTCGAGGATATCAAGAAGGAGCTGACATCCCTGGGCTTT |
| | | CTGGAGTCCGGCTCTGACCCCGGACAGCTGAAGCACGTGGTGGAT |
| | | GTGACCGACACAGTGCGGAAGGATGTGGAGGAGTGGGGCCCTTT |
| | | CGACCTGGTGTACGGAGCAACCCCTCCACTGGGACACACATGCGA |
| | | CAGACCCCCTTCTTGGTACCTGTTCCAGTTTCACCGCCTGCTGCAGT |
| | | ATGCAAGGCCAAAGCCAGGCAGCCCTAGACCATTCTTTTGGATGTT |
| | | CGTGGATAATCTGGTGCTGAACAAGGAGGATCTGGACGTGGCCA |
| | | GCAGGTTTCTGGAGATGGAGCCAGTGACCATCCCAGACGTGCACG |
| | | GCGGCTCCCTGCAGAATGCCGTGCGCGTGTGGTCTAACATCCCTGC |
| | | CATCAGAAGCAGGCACTGGGCACTGGTGAGCGAGGAGGAGCTGT |
| | | CCCTGCTGGCCCAGAATAAGCAGAGCAGCAAGCTGGCCGCCAAGT |
| | | GGCCTACAAAGCTGGTGAAGAACTGCTTCCTGCCACTGCGGGAGT |
| | | ACTTCAAGTATTTTTCCACCGAGCTGACATCTAGCCTGGGAGGACC |
| | | CTCCTCTGGCGCCCCACCACCTAGCGGCGGCTCCCCTGCCGGCTCT |
| | | CCAACCAGCACAGAGGAGGGCACCAGCGAGTCCGCCACACCAGA |
| | | GTCTGGACCTGGCACCAGCACAGAGCCATCCGAGGGCTCTGCCCC |
| | | GGGCTCTCCTGCAGGCAGCCCTACCTCCACCGAAGAGGGCACCAG |
| | | CACAGAGCCTTCTGAGGGCAGCGCCCCAGGCACCTCTACAGAGCC |
| | | AAGCGAGCTCGAGGACAAGAAGTACAGCATCGGCCTGGCCATCG |
| | | GCACCAACTCTGTGGGCTGGGCCGTGATCACCGACGAGTACAAGG |
| | | TGCCCAGCAAGAAATTCAAGGTGCTGGGCAACACCGACCGGCACA |
| | | GCATCAAGAAGAACCTGATCGGAGCCCTGCTGTTCGACAGCGGCG |
| | | AAACAGCCGAGGCCACCCGGCTGAAGAGAACCGCCAGAAGAAGA |
| | | TACACCAGACGGAAGAACCGGATCTGCTATCTGCAAGAGATCTTC |
| | | AGCAACGAGATGGCCAAGGTGGACGACAGCTTCTTCCACAGACTG |
| | | GAAGAGTCCTTCCTGGTGGAAGAGGATAAGAAGCACGAGCGGCA |
| | | CCCCATCTTCGGCAACATCGTGGACGAGGTGGCCTACCACGAGAA |
| | | GTACCCCACCATCTACCACCTGAGAAAGAAACTGGTGGACAGCAC |
| | | CGACAAGGCCGACCTGCGGCTGATCTATCTGGCCCTGGCCCACAT |
| | | GATCAAGTTCCGGGGCCACTTCCTGATCGAGGGCGACCTGAACCC |
| | | CGACAACAGCGACGTGGACAAGCTGTTCATCCAGCTGGTGCAGAC |
| | | CTACAACCAGCTGTTCGAGGAAAACCCCATCAACGCCAGCGGCGT |
| | | GGACGCCAAGGCCATCCTGTCTGCCAGACTGAGCAAGAGCAGACG |
| | | GCTGGAAAATCTGATCGCCCAGCTGCCCGGCGAGAAGAAGAATG |
| | | GCCTGTTCGGCAACCTGATTGCCCTGAGCCTGGGCCTGACCCCCAA |
| | | CTTCAAGAGCAACTTCGACCTGGCCGAGGATGCCAAACTGCAGCT |
| | | GAGCAAGGACACCTACGACGACGACCTGGACAACCTGCTGGCCCA |
| | | GATCGGCGACCAGTACGCCGACCTGTTTCTGGCCGCCAAGAACCT |
| | | GTCCGACGCCATCCTGCTGAGCGACATCCTGAGAGTGAACACCGA |
| | | GATCACCAAGGCCCCCCTGAGCGCCTCTATGATCAAGAGATACGA |
| | | CGAGCACCACCAGGACCTGACCCTGCTGAAAGCTCTCGTGCGGCA |
| | | GCAGCTGCCTGAGAAGTACAAAGAGATTTTCTTCGACCAGAGCAA |
| | | GAACGGCTACGCCGGCTACATTGACGGCGGAGCCAGCCAGGAAG |
| | | AGTTCTACAAGTTCATCAAGCCCATCCTGGAAAAGATGGACGGCA |
| | | CCGAGGAACTGCTCGTGAAGCTGAACAGAGAGGACCTGCTGCGG |
| | | AAGCAGCGGACCTTCGACAACGGCAGCATCCCCCACCAGATCCAC |
| | | CTGGGAGAGCTGCACGCCATTCTGCGGCGGCAGGAAGATTTTTAC |
| | | CCATTCCTGAAGGACAACCGGGAAAAGATCGAGAAGATCCTGACC |
| | | TTCCGCATCCCCTACTACGTGGGCCCTCTGGCCAGGGGAAACAGC |
| | | AGATTCGCCTGGATGACCAGAAAGAGCGAGGAAACCATCACCCCC |
| | | TGGAACTTCGAGGAAGTGGTGGACAAGGGCGCTTCCGCCCAGAG |
| | | CTTCATCGAGCGGATGACCAACTTCGATAAGAACCTGCCCAACGA |
| | | GAAGGTGCTGCCCAAGCACAGCCTGCTGTACGAGTACTTCACCGT |
| | | GTATAACGAGCTGACCAAAGTGAAATACGTGACCGAGGGAATGA |
| | | GAAAGCCCGCCTTCCTGAGCGGCGAGCAGAAAAAGGCCATCGTG |
| | | GACCTGCTGTTCAAGACCAACCGGAAAGTGACCGTGAAGCAGCTG |
| | | AAAGAGGACTACTTCAAGAAAATCGAGTGCTTCGACTCCGTGGAA |
| | | ATCTCCGGCGTGGAAGATCGGTTCAACGCCTCCCTGGGCACATACC |
| | | ACGATCTGCTGAAAATTATCAAGGACAAGGACTTCCTGGACAATG |
| | | AGGAAAACGAGGACATTCTGGAAGATATCGTGCTGACCCTGACAC |
| | | TGTTTGAGGACAGAGAGATGATCGAGGAACGGCTGAAAACCTAT |
| | | GCCCACCTGTTCGACGACAAAGTGATGAAGCAGCTGAAGCGGCG |
| | | GAGATACACCGGCTGGGGCAGGCTGAGCCGGAAGCTGATCAACG |
| | | GCATCCGGGACAAGCAGTCCGGCAAGACAATCCTGGATTTCCTGA |
| | | AGTCCGACGGCTTCGCCAACAGAAACTTCATGCAGCTGATCCACG |
| | | ACGACAGCCTGACCTTTAAAGAGGACATCCAGAAAGCCCAGGTGT |
| | | CCGGCCAGGGCGATAGCCTGCACGAGCACATTGCCAATCTGGCCG |
| | | GCAGCCCCGCCATTAAGAAGGGCATCCTGCAGACAGTGAAGGTG |
| | | GTGGACGAGCTCGTGAAAGTGATGGGCCGGCACAAGCCCGAGAA |
| | | CATCGTGATCGAAATGGCCAGAGAGAACCAGACCACCCAGAAGG |
| | | GACAGAAGAACAGCCGCGAGAGAATGAAGCGGATCGAAGAGGG |
| | | CATCAAAGAGCTGGGCAGCCAGATCCTGAAAGAACACCCCGTGGA |
| | | AAACACCCAGCTGCAGAACGAGAAGCTGTACCTGTACTACCTGCA |
| | | GAATGGGCGGGATATGTACGTGGACCAGGAACTGGACATCAACC |
| | | GGCTGTCCGACTACGATGTGGACGCCATCGTGCCTCAGAGCTTTCT |
| | | GAAGGACGACTCCATCGACAACAAGGTGCTGACCAGAAGCGACA |
| | | AGAACCGGGGCAAGAGCGACAACGTGCCCTCCGAAGAGGTCGTG |
| | | AAGAAGATGAAGAACTACTGGCGGCAGCTGCTGAACGCCAAGCT |
| | | GATTACCCAGAGAAAGTTCGACAATCTGACCAAGGCCGAGAGAGG |
| | | CGGCCTGAGCGAACTGGATAAGGCCGGCTTCATCAAGAGACAGCT |
| | | GGTGGAAACCCGGCAGATCACAAAGCACGTGGCACAGATCCTGG |
| | | ACTCCCGGATGAACACTAAGTACGACGAGAATGACAAGCTGATCC |
| | | GGGAAGTGAAAGTGATCACCCTGAAGTCCAAGCTGGTGTCCGATT |
| | | TCCGGAAGGATTTCCAGTTTTACAAAGTGCGCGAGATCAACAACTA |
| | | CCACCACGCCCACGACGCCTACCTGAACGCCGTCGTGGGAACCGC |
| | | CCTGATCAAAAAGTACCCTAAGCTGGAAAGCGAGTTCGTGTACGG |
| | | CGACTACAAGGTGTACGACGTGCGGAAGATGATCGCCAAGAGCG |
| | | AGCAGGAAATCGGCAAGGCTACCGCCAAGTACTTCTTCTACAGCA |
| | | ACATCATGAACTTTTTCAAGACCGAGATTACCCTGGCCAACGGCGA |
| | | GATCCGGAAGCGGCCTCTGATCGAGACAAACGGCGAAACCGGGG |
| | | AGATCGTGTGGGATAAGGGCCGGGATTTTGCCACCGTGCGGAAA |
| | | GTGCTGAGCATGCCCCAAGTGAATATCGTGAAAAAGACCGAGGTG |
| | | CAGACAGGCGGCTTCAGCAAAGAGTCTATCCTGCCCAAGAGGAAC |
| | | AGCGATAAGCTGATCGCCAGAAAGAAGGACTGGGACCCTAAGAA |
| | | GTACGGCGGCTTCGACAGCCCCACCGTGGCCTATTCTGTGCTGGTG |
| | | GTGGCCAAAGTGGAAAAGGGCAAGTCCAAGAAACTGAAGAGTGT |
| | | GAAAGAGCTGCTGGGGATCACCATCATGGAAAGAAGCAGCTTCGA |
| | | GAAGAATCCCATCGACTTTCTGGAAGCCAAGGGCTACAAAGAAGT |
| | | GAAAAAGGACCTGATCATCAAGCTGCCTAAGTACTCCCTGTTCGAG |
| | | CTGGAAAACGGCCGGAAGAGAATGCTGGCCTCTGCCGGCGAACT |
| | | GCAGAAGGGAAACGAACTGGCCCTGCCCTCCAAATATGTGAACTT |
| | | CCTGTACCTGGCCAGCCACTATGAGAAGCTGAAGGGCTCCCCCGA |
| | | GGATAATGAGCAGAAACAGCTGTTTGTGGAACAGCACAAGCACTA |
| | | CCTGGACGAGATCATCGAGCAGATCAGCGAGTTCTCCAAGAGAGT |
| | | GATCCTGGCCGACGCTAATCTGGACAAAGTGCTGTCCGCCTACAAC |
| | | AAGCACCGGGATAAGCCCATCAGAGAGCAGGCCGAGAATATCATC |
| | | CACCTGTTTACCCTGACCAATCTGGGAGCCCCTGCCGCCTTCAAGT |
| | | ACTTTGACACCACCATCGACCGGAAGAGGTACACCAGCACCAAAG |
| | | AGGTGCTGGACGCCACCCTGATCCACCAGAGCATCACCGGCCTGT |
| | | ACGAGACACGGATCGACCTGTCTCAGCTGGGAGGCGACAGCCCCA |
| | | AGAAGAAGAGAAAGGTGGGAGTCGACGGATCCAGCGGCTCCGAG |
| | | ACCCCAGGCACATCTGAGAGCGCCACCCCTGAGTCCACCGGTAAC |
| | | AAAAAGCTTGAGGCCGTCGGAACCGGAATCGAACCAAAAGCAAT |
| | | GTCCCAGGGTTTGGTGACATTTGGCGACGTGGCTGTCGATTTTTCC |
| | | CAGGAAGAGTGGGAGTGGCTCAATCCTATCCAGAGGAACTTGTAC |
| | | CGGAAGGTGATGCTGGAGAATTATAGAAATTTGGCATCACTGGGG |
| | | TTGTGCGTTAGCAAACCAGATGTTATATCTTCCCTGGAACAGGGAA |
| | | AGGAGCCCTGGAGCGCTGATTACAAAGATGATGACGATAAAGCCC |
| | | CCAAGAAGAAAAGGAAGGTCCCAAAGAAAAAAAGAAAGGTGTGA |
| | Fusion Protein | MGTMPKKKRKVPKKKRKVYNHDQEFDPPKVYPPVPAEKRKPIRVLSL |
| | 11 Amino Acid | FDGIATGLLVLKDLGIQVDRYIASEVCEDSITVGMVRHQGKIMYVGDV |
| | Sequence | RSVTQKHIQEWGPFDLVIGGSPCNDLSIVNPARKGLYEGTGRLFFEFY |
| | NLS-NLS-3A- | RLLHDARPKEGDDRPFFWLFENVVAMGVSDKRDISRFLESNPVMIDA |
| | ADD-h3L-dCas9- | KEVSAAHRARYFWGNLPGMNRPLASTVNDKLELQECLEHGRIAKFSK |
| | ZIM3-NLS-NLS | VRTITTRSNSIKQGKDQHFPVFMNEKEDILWCTEMERVFGFPVHYTD |
| | | VSNMSRLARQRLLGRSWSVPVIRHLFAPLKEYFACVSSGNSNANSRG |
| | | PSFSSGLVPLSLRGSHMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSLGGPSSGAPPPSGGSPAGSPTSTEEGTSES |
| | | ATPESGPGTSTEPSEGSAPGSPAGSPTSTEEGTSTEPSEGSAPGTSTEP |
| | | SELEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSSGSETPGTSESATPESTGMNNSQGRVTFEDVTVNF |
| | | TQGEWQRLNPEQRNLYRDVMLENYSNLVSVGQGETTKPDVILRLEQ |
| | | GKEPWLEEEEVLGSGRAEKNGDIGGQIWKPKDVKESLSADYKDDDD |
| | | KAPKKKRKVPKKKRKV |
| | Fusion Protein | ATGGGTACCATGCCAAAAAAGAAGAGAAAGGTACCGAAGAAAAA |
| | 11 DNA | AAGAAAGGTATACAATCACGATCAGGAGTTCGACCCCCCTAAGGT |
| | Sequence | GTACCCACCAGTGCCTGCAGAGAAGAGGAAGCCAATCCGGGTGCT |
| | | GAGCCTGTTTGATGGCATCGCCACCGGCCTGCTGGTGCTGAAGGA |
| | | TCTGGGCATCCAGGTGGACCGGTACATCGCCTCCGAGGTGTGCGA |
| | | GGATTCTATCACCGTGGGCATGGTGCGCCACCAGGGCAAGATCAT |
| | | GTATGTGGGCGACGTGCGGTCCGTGACACAGAAGCACATCCAGG |
| | | AGTGGGGCCCATTCGATCTGGTGATCGGCGGCAGCCCCTGTAATG |
| | | ACCTGTCCATCGTGAACCCTGCAAGGAAGGGACTGTACGAGGGAA |
| | | CCGGCCGGCTGTTCTTTGAGTTTTATAGACTGCTGCACGACGCCAG |
| | | GCCTAAGGAGGGCGACGATAGACCATTCTTTTGGCTGTTCGAGAA |
| | | TGTGGTGGCTATGGGCGTGAGCGATAAGAGGGACATCTCCAGGTT |
| | | TCTGGAGTCTAACCCCGTGATGATCGATGCAAAGGAGGTGTCCGC |
| | | CGCACACAGAGCCAGGTATTTCTGGGGCAATCTGCCAGGAATGAA |
| | | CAGGCCACTGGCAAGCACCGTGAATGACAAGCTGGAGCTGCAGG |
| | | AGTGCCTGGAGCACGGAAGGATCGCCAAGTTTTCCAAGGTGCGCA |
| | | CAATCACCACACGGAGCAATTCCATCAAGCAGGGCAAGGATCAGC |
| | | ACTTCCCCGTGTTCATGAACGAGAAGGAGGACATCCTGTGGTGTA |
| | | CCGAGATGGAGAGAGTGTTCGGCTTTCCAGTGCACTACACAGACG |
| | | TGTCTAACATGAGCAGGCTGGCAAGGCAGCGGCTGCTGGGCAGA |
| | | TCTTGGAGCGTGCCCGTGATCAGGCACCTGTTCGCCCCTCTGAAGG |
| | | AGTATTTTGCCTGCGTGAGCAGCGGCAACTCCAATGCCAACAGCC |
| | | GGGGCCCCTCTTTCAGCTCCGGATTGGTGCCTCTGAGCCTGAGGG |
| | | GCTCCCACATGGCAGCAATCCCCGCCCTGGACCCCGAGGCCGAGC |
| | | CTAGCATGGACGTGATCCTGGTGGGCTCTAGCGAGCTGTCCTCTA |
| | | GCGTGTCTCCAGGAACCGGAAGGGATCTGATCGCATACGAGGTGA |
| | | AGGCCAATCAGCGGAACATCGAGGACATCTGTATCTGCTGTGGCA |
| | | GCCTGCAGGTGCACACACAGCACCCACTGTTCGAGGGAGGAATCT |
| | | GCGCACCCTGTAAGGATAAGTTCCTGGACGCCCTGTTTCTGTACGA |
| | | CGATGACGGCTACCAGTCCTATTGCTCTATCTGCTGTTCCGGCGAG |
| | | ACCCTGCTGATCTGCGGCAATCCAGATTGTACAAGGTGCTATTGTT |
| | | TTGAGTGCGTGGACTCTCTGGTGGGACCAGGCACCAGCGGAAAG |
| | | GTGCACGCCATGTCCAACTGGGTGTGCTACCTGTGCCTGCCATCCT |
| | | CTCGCAGCGGACTGCTGCAGCGGAGAAGGAAGTGGAGATCCCAG |
| | | CTGAAGGCCTTCTATGATAGGGAGTCTGAGAACCCCCTGGAGATG |
| | | TTTGAGACCGTGCCAGTGTGGCGCCGGCAGCCCGTGAGGGTGCTG |
| | | AGCCTGTTCGAGGATATCAAGAAGGAGCTGACATCCCTGGGCTTT |
| | | CTGGAGTCCGGCTCTGACCCCGGACAGCTGAAGCACGTGGTGGAT |
| | | GTGACCGACACAGTGCGGAAGGATGTGGAGGAGTGGGGCCCTTT |
| | | CGACCTGGTGTACGGAGCAACCCCTCCACTGGGACACACATGCGA |
| | | CAGACCCCCTTCTTGGTACCTGTTCCAGTTTCACCGCCTGCTGCAGT |
| | | ATGCAAGGCCAAAGCCAGGCAGCCCTAGACCATTCTTTTGGATGTT |
| | | CGTGGATAATCTGGTGCTGAACAAGGAGGATCTGGACGTGGCCA |
| | | GCAGGTTTCTGGAGATGGAGCCAGTGACCATCCCAGACGTGCACG |
| | | GCGGCTCCCTGCAGAATGCCGTGCGCGTGTGGTCTAACATCCCTGC |
| | | CATCAGAAGCAGGCACTGGGCACTGGTGAGCGAGGAGGAGCTGT |
| | | CCCTGCTGGCCCAGAATAAGCAGAGCAGCAAGCTGGCCGCCAAGT |
| | | GGCCTACAAAGCTGGTGAAGAACTGCTTCCTGCCACTGCGGGAGT |
| | | ACTTCAAGTATTTTTCCACCGAGCTGACATCTAGCCTGGGAGGACC |
| | | CTCCTCTGGCGCCCCACCACCTAGCGGCGGCTCCCCTGCCGGCTCT |
| | | CCAACCAGCACAGAGGAGGGCACCAGCGAGTCCGCCACACCAGA |
| | | GTCTGGACCTGGCACCAGCACAGAGCCATCCGAGGGCTCTGCCCC |
| | | AGGCTCTCCTGCAGGCAGCCCTACCTCCACCGAAGAGGGCACCAG |
| | | CACAGAGCCTTCTGAGGGCAGCGCCCCAGGCACCTCTACAGAGCC |
| | | AAGCGAGCTCGAGGACAAGAAGTACAGCATCGGCCTGGCCATCG |
| | | GCACCAACTCTGTGGGCTGGGCCGTGATCACCGACGAGTACAAGG |
| | | TGCCCAGCAAGAAATTCAAGGTGCTGGGCAACACCGACCGGCACA |
| | | GCATCAAGAAGAACCTGATCGGAGCCCTGCTGTTCGACAGCGGCG |
| | | AAACAGCCGAGGCCACCCGGCTGAAGAGAACCGCCAGAAGAAGA |
| | | TACACCAGACGGAAGAACCGGATCTGCTATCTGCAAGAGATCTTC |
| | | AGCAACGAGATGGCCAAGGTGGACGACAGCTTCTTCCACAGACTG |
| | | GAAGAGTCCTTCCTGGTGGAAGAGGATAAGAAGCACGAGCGGCA |
| | | CCCCATCTTCGGCAACATCGTGGACGAGGTGGCCTACCACGAGAA |
| | | GTACCCCACCATCTACCACCTGAGAAAGAAACTGGTGGACAGCAC |
| | | CGACAAGGCCGACCTGCGGCTGATCTATCTGGCCCTGGCCCACAT |
| | | GATCAAGTTCCGGGGCCACTTCCTGATCGAGGGCGACCTGAACCC |
| | | CGACAACAGCGACGTGGACAAGCTGTTCATCCAGCTGGTGCAGAC |
| | | CTACAACCAGCTGTTCGAGGAAAACCCCATCAACGCCAGCGGCGT |
| | | GGACGCCAAGGCCATCCTGTCTGCCAGACTGAGCAAGAGCAGACG |
| | | GCTGGAAAATCTGATCGCCCAGCTGCCCGGCGAGAAGAAGAATG |
| | | GCCTGTTCGGCAACCTGATTGCCCTGAGCCTGGGCCTGACCCCCAA |
| | | CTTCAAGAGCAACTTCGACCTGGCCGAGGATGCCAAACTGCAGCT |
| | | GAGCAAGGACACCTACGACGACGACCTGGACAACCTGCTGGCCCA |
| | | GATCGGCGACCAGTACGCCGACCTGTTTCTGGCCGCCAAGAACCT |
| | | GTCCGACGCCATCCTGCTGAGCGACATCCTGAGAGTGAACACCGA |
| | | GATCACCAAGGCCCCCCTGAGCGCCTCTATGATCAAGAGATACGA |
| | | CGAGCACCACCAGGACCTGACCCTGCTGAAAGCTCTCGTGCGGCA |
| | | GCAGCTGCCTGAGAAGTACAAAGAGATTTTCTTCGACCAGAGCAA |
| | | GAACGGCTACGCCGGCTACATTGACGGCGGAGCCAGCCAGGAAG |
| | | AGTTCTACAAGTTCATCAAGCCCATCCTGGAAAAGATGGACGGCA |
| | | CCGAGGAACTGCTCGTGAAGCTGAACAGAGAGGACCTGCTGCGG |
| | | AAGCAGCGGACCTTCGACAACGGCAGCATCCCCCACCAGATCCAC |
| | | CTGGGAGAGCTGCACGCCATTCTGCGGCGGCAGGAAGATTTTTAC |
| | | CCATTCCTGAAGGACAACCGGGAAAAGATCGAGAAGATCCTGACC |
| | | TTCCGCATCCCCTACTACGTGGGCCCTCTGGCCAGGGGAAACAGC |
| | | AGATTCGCCTGGATGACCAGAAAGAGCGAGGAAACCATCACCCCC |
| | | TGGAACTTCGAGGAAGTGGTGGACAAGGGCGCTTCCGCCCAGAG |
| | | CTTCATCGAGCGGATGACCAACTTCGATAAGAACCTGCCCAACGA |
| | | GAAGGTGCTGCCCAAGCACAGCCTGCTGTACGAGTACTTCACCGT |
| | | GTATAACGAGCTGACCAAAGTGAAATACGTGACCGAGGGAATGA |
| | | GAAAGCCCGCCTTCCTGAGCGGCGAGCAGAAAAAGGCCATCGTG |
| | | GACCTGCTGTTCAAGACCAACCGGAAAGTGACCGTGAAGCAGCTG |
| | | AAAGAGGACTACTTCAAGAAAATCGAGTGCTTCGACTCCGTGGAA |
| | | ATCTCCGGCGTGGAAGATCGGTTCAACGCCTCCCTGGGCACATACC |
| | | ACGATCTGCTGAAAATTATCAAGGACAAGGACTTCCTGGACAATG |
| | | AGGAAAACGAGGACATTCTGGAAGATATCGTGCTGACCCTGACAC |
| | | TGTTTGAGGACAGAGAGATGATCGAGGAACGGCTGAAAACCTAT |
| | | GCCCACCTGTTCGACGACAAAGTGATGAAGCAGCTGAAGCGGCG |
| | | GAGATACACCGGCTGGGGCAGGCTGAGCCGGAAGCTGATCAACG |
| | | GCATCCGGGACAAGCAGTCCGGCAAGACAATCCTGGATTTCCTGA |
| | | AGTCCGACGGCTTCGCCAACAGAAACTTCATGCAGCTGATCCACG |
| | | ACGACAGCCTGACCTTTAAAGAGGACATCCAGAAAGCCCAGGTGT |
| | | CCGGCCAGGGCGATAGCCTGCACGAGCACATTGCCAATCTGGCCG |
| | | GCAGCCCCGCCATTAAGAAGGGCATCCTGCAGACAGTGAAGGTG |
| | | GTGGACGAGCTCGTGAAAGTGATGGGCCGGCACAAGCCCGAGAA |
| | | CATCGTGATCGAAATGGCCAGAGAGAACCAGACCACCCAGAAGG |
| | | GACAGAAGAACAGCCGCGAGAGAATGAAGCGGATCGAAGAGGG |
| | | CATCAAAGAGCTGGGCAGCCAGATCCTGAAAGAACACCCCGTGGA |
| | | AAACACCCAGCTGCAGAACGAGAAGCTGTACCTGTACTACCTGCA |
| | | GAATGGGCGGGATATGTACGTGGACCAGGAACTGGACATCAACC |
| | | GGCTGTCCGACTACGATGTGGACGCCATCGTGCCTCAGAGCTTTCT |
| | | GAAGGACGACTCCATCGACAACAAGGTGCTGACCAGAAGCGACA |
| | | AGAACCGGGGCAAGAGCGACAACGTGCCCTCCGAAGAGGTCGTG |
| | | AAGAAGATGAAGAACTACTGGCGGCAGCTGCTGAACGCCAAGCT |
| | | GATTACCCAGAGAAAGTTCGACAATCTGACCAAGGCCGAGAGAGG |
| | | CGGCCTGAGCGAACTGGATAAGGCCGGCTTCATCAAGAGACAGCT |
| | | GGTGGAAACCCGGCAGATCACAAAGCACGTGGCACAGATCCTGG |
| | | ACTCCCGGATGAACACTAAGTACGACGAGAATGACAAGCTGATCC |
| | | GGGAAGTGAAAGTGATCACCCTGAAGTCCAAGCTGGTGTCCGATT |
| | | TCCGGAAGGATTTCCAGTTTTACAAAGTGCGCGAGATCAACAACTA |
| | | CCACCACGCCCACGACGCCTACCTGAACGCCGTCGTGGGAACCGC |
| | | CCTGATCAAAAAGTACCCTAAGCTGGAAAGCGAGTTCGTGTACGG |
| | | CGACTACAAGGTGTACGACGTGCGGAAGATGATCGCCAAGAGCG |
| | | AGCAGGAAATCGGCAAGGCTACCGCCAAGTACTTCTTCTACAGCA |
| | | ACATCATGAACTTTTTCAAGACCGAGATTACCCTGGCCAACGGCGA |
| | | GATCCGGAAGCGGCCTCTGATCGAGACAAACGGCGAAACCGGGG |
| | | AGATCGTGTGGGATAAGGGCCGGGATTTTGCCACCGTGCGGAAA |
| | | GTGCTGAGCATGCCCCAAGTGAATATCGTGAAAAAGACCGAGGTG |
| | | CAGACAGGCGGCTTCAGCAAAGAGTCTATCCTGCCCAAGAGGAAC |
| | | AGCGATAAGCTGATCGCCAGAAAGAAGGACTGGGACCCTAAGAA |
| | | GTACGGCGGCTTCGACAGCCCCACCGTGGCCTATTCTGTGCTGGTG |
| | | GTGGCCAAAGTGGAAAAGGGCAAGTCCAAGAAACTGAAGAGTGT |
| | | GAAAGAGCTGCTGGGGATCACCATCATGGAAAGAAGCAGCTTCGA |
| | | GAAGAATCCCATCGACTTTCTGGAAGCCAAGGGCTACAAAGAAGT |
| | | GAAAAAGGACCTGATCATCAAGCTGCCTAAGTACTCCCTGTTCGAG |
| | | CTGGAAAACGGCCGGAAGAGAATGCTGGCCTCTGCCGGCGAACT |
| | | GCAGAAGGGAAACGAACTGGCCCTGCCCTCCAAATATGTGAACTT |
| | | CCTGTACCTGGCCAGCCACTATGAGAAGCTGAAGGGCTCCCCCGA |
| | | GGATAATGAGCAGAAACAGCTGTTTGTGGAACAGCACAAGCACTA |
| | | CCTGGACGAGATCATCGAGCAGATCAGCGAGTTCTCCAAGAGAGT |
| | | GATCCTGGCCGACGCTAATCTGGACAAAGTGCTGTCCGCCTACAAC |
| | | AAGCACCGGGATAAGCCCATCAGAGAGCAGGCCGAGAATATCATC |
| | | CACCTGTTTACCCTGACCAATCTGGGAGCCCCTGCCGCCTTCAAGT |
| | | ACTTTGACACCACCATCGACCGGAAGAGGTACACCAGCACCAAAG |
| | | AGGTGCTGGACGCCACCCTGATCCACCAGAGCATCACCGGCCTGT |
| | | ACGAGACACGGATCGACCTGTCTCAGCTGGGAGGCGACAGCCCCA |
| | | AGAAGAAGAGAAAGGTGGGAGTCGACGGATCCAGCGGCTCCGAG |
| | | ACCCCAGGCACATCTGAGAGCGCCACCCCTGAGTCCACCGGTATG |
| | | AACAATTCACAGGGGAGAGTGACATTCGAAGACGTGACCGTGAAC |
| | | TTCACCCAGGGAGAATGGCAGCGCTTGAACCCAGAACAAAGGAAC |
| | | CTCTATCGGGACGTGATGCTGGAAAACTACTCAAATTTGGTGAGC |
| | | GTTGGGCAGGGTGAGACCACTAAGCCTGACGTGATCCTGAGATTG |
| | | GAACAGGGCAAGGAGCCTTGGCTCGAGGAAGAGGAAGTCCTGGG |
| | | CTCAGGGAGGGCCGAGAAAAACGGTGATATAGGAGGCCAGATAT |
| | | GGAAGCCTAAGGACGTCAAGGAGAGCCTGAGCGCTGATTACAAA |
| | | GATGATGACGATAAAGCCCCCAAGAAGAAAAGGAAGGTCCCAAA |
| | | GAAAAAAAGAAAGGTGTGA |
| | Fusion Protein | MGTMPKKKRKVPKKKRKVYNHDQEFDPPKVYPPVPAEKRKPIRVLSL |
| | 12 Amino Acid | FDGIATGLLVLKDLGIQVDRYIASEVCEDSITVGMVRHQGKIMYVGDV |
| | Sequence | RSVTQKHIQEWGPFDLVIGGSPCNDLSIVNPARKGLYEGTGRLFFEFY |
| | NLS-NLS-3A- | RLLHDARPKEGDDRPFFWLFENVVAMGVSDKRDISRFLESNPVMIDA |
| | ADD-h3L-dCas9- | KEVSAAHRARYFWGNLPGMNRPLASTVNDKLELQECLEHGRIAKFSK |
| | ZN627-NLS-NLS | VRTITTRSNSIKQGKDQHFPVFMNEKEDILWCTEMERVFGFPVHYTD |
| | | VSNMSRLARQRLLGRSWSVPVIRHLFAPLKEYFACVSSGNSNANSRG |
| | | PSFSSGLVPLSLRGSHMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSLGGPSSGAPPPSGGSPAGSPTSTEEGTSES |
| | | ATPESGPGTSTEPSEGSAPGSPAGSPTSTEEGTSTEPSEGSAPGTSTEP |
| | | SELEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSSGSETPGTSESATPESTGDSVAFEDVAVNFTLEEW |
| | | ALLDPSQKNLYRDVMRETFRNLASVGKQWEDQNIEDPFKIPRRNISHI |
| | | PERLCESKEGGQGEESADYKDDDDKAPKKKRKVPKKKRKV |
| | Fusion Protein | ATGGGTACCATGCCAAAAAAGAAGAGAAAGGTACCGAAGAAAAA |
| | 12 DNA | AAGAAAGGTATACAATCACGATCAGGAGTTCGACCCCCCTAAGGT |
| | Sequence | GTACCCACCAGTGCCTGCAGAGAAGAGGAAGCCAATCCGGGTGCT |
| | | GAGCCTGTTTGATGGCATCGCCACCGGCCTGCTGGTGCTGAAGGA |
| | | TCTGGGCATCCAGGTGGACCGGTACATCGCCTCCGAGGTGTGCGA |
| | | GGATTCTATCACCGTGGGCATGGTGCGCCACCAGGGCAAGATCAT |
| | | GTATGTGGGCGACGTGCGGTCCGTGACACAGAAGCACATCCAGG |
| | | AGTGGGGCCCATTCGATCTGGTGATCGGCGGCAGCCCCTGTAATG |
| | | ACCTGTCCATCGTGAACCCTGCAAGGAAGGGACTGTACGAGGGAA |
| | | CCGGCCGGCTGTTCTTTGAGTTTTATAGACTGCTGCACGACGCCAG |
| | | GCCTAAGGAGGGCGACGATAGACCATTCTTTTGGCTGTTCGAGAA |
| | | TGTGGTGGCTATGGGCGTGAGCGATAAGAGGGACATCTCCAGGTT |
| | | TCTGGAGTCTAACCCCGTGATGATCGATGCAAAGGAGGTGTCCGC |
| | | CGCACACAGAGCCAGGTATTTCTGGGGCAATCTGCCAGGAATGAA |
| | | CAGGCCACTGGCAAGCACCGTGAATGACAAGCTGGAGCTGCAGG |
| | | AGTGCCTGGAGCACGGAAGGATCGCCAAGTTTTCCAAGGTGCGCA |
| | | CAATCACCACACGGAGCAATTCCATCAAGCAGGGCAAGGATCAGC |
| | | ACTTCCCCGTGTTCATGAACGAGAAGGAGGACATCCTGTGGTGTA |
| | | CCGAGATGGAGAGAGTGTTCGGCTTTCCAGTGCACTACACAGACG |
| | | TGTCTAACATGAGCAGGCTGGCAAGGCAGCGGCTGCTGGGCAGA |
| | | TCTTGGAGCGTGCCCGTGATCAGGCACCTGTTCGCCCCTCTGAAGG |
| | | AGTATTTTGCCTGCGTGAGCAGCGGCAACTCCAATGCCAACAGCC |
| | | GGGGCCCCTCTTTCAGCTCCGGATTGGTGCCTCTGAGCCTGAGGG |
| | | GCTCCCACATGGCAGCAATCCCCGCCCTGGACCCCGAGGCCGAGC |
| | | CTAGCATGGACGTGATCCTGGTGGGCTCTAGCGAGCTGTCCTCTA |
| | | GCGTGTCTCCAGGAACCGGAAGGGATCTGATCGCATACGAGGTGA |
| | | AGGCCAATCAGCGGAACATCGAGGACATCTGTATCTGCTGTGGCA |
| | | GCCTGCAGGTGCACACACAGCACCCACTGTTCGAGGGAGGAATCT |
| | | GCGCACCCTGTAAGGATAAGTTCCTGGACGCCCTGTTTCTGTACGA |
| | | CGATGACGGCTACCAGTCCTATTGCTCTATCTGCTGTTCCGGCGAG |
| | | ACCCTGCTGATCTGCGGCAATCCAGATTGTACAAGGTGCTATTGTT |
| | | TTGAGTGCGTGGACTCTCTGGTGGGACCAGGCACCAGCGGAAAG |
| | | GTGCACGCCATGTCCAACTGGGTGTGCTACCTGTGCCTGCCATCCT |
| | | CTCGCAGCGGACTGCTGCAGCGGAGAAGGAAGTGGAGATCCCAG |
| | | CTGAAGGCCTTCTATGATAGGGAGTCTGAGAACCCCCTGGAGATG |
| | | TTTGAGACCGTGCCAGTGTGGCGCCGGCAGCCCGTGAGGGTGCTG |
| | | AGCCTGTTCGAGGATATCAAGAAGGAGCTGACATCCCTGGGCTTT |
| | | CTGGAGTCCGGCTCTGACCCCGGACAGCTGAAGCACGTGGTGGAT |
| | | GTGACCGACACAGTGCGGAAGGATGTGGAGGAGTGGGGCCCTTT |
| | | CGACCTGGTGTACGGAGCAACCCCTCCACTGGGACACACATGCGA |
| | | CAGACCCCCTTCTTGGTACCTGTTCCAGTTTCACCGCCTGCTGCAGT |
| | | ATGCAAGGCCAAAGCCAGGCAGCCCTAGACCATTCTTTTGGATGTT |
| | | CGTGGATAATCTGGTGCTGAACAAGGAGGATCTGGACGTGGCCA |
| | | GCAGGTTTCTGGAGATGGAGCCAGTGACCATCCCAGACGTGCACG |
| | | GCGGCTCCCTGCAGAATGCCGTGCGCGTGTGGTCTAACATCCCTGC |
| | | CATCAGAAGCAGGCACTGGGCACTGGTGAGCGAGGAGGAGCTGT |
| | | CCCTGCTGGCCCAGAATAAGCAGAGCAGCAAGCTGGCCGCCAAGT |
| | | GGCCTACAAAGCTGGTGAAGAACTGCTTCCTGCCACTGCGGGAGT |
| | | ACTTCAAGTATTTTTCCACCGAGCTGACATCTAGCCTGGGAGGACC |
| | | CTCCTCTGGCGCCCCACCACCTAGCGGCGGCTCCCCTGCCGGCTCT |
| | | CCAACCAGCACAGAGGAGGGCACCAGCGAGTCCGCCACACCAGA |
| | | GTCTGGACCTGGCACCAGCACAGAGCCATCCGAGGGCTCTGCCCC |
| | | AGGCTCTCCTGCAGGCAGCCCTACCTCCACCGAAGAGGGCACCAG |
| | | CACAGAGCCTTCTGAGGGCAGCGCCCCAGGCACCTCTACAGAGCC |
| | | AAGCGAGCTCGAGGACAAGAAGTACAGCATCGGCCTGGCCATCG |
| | | GCACCAACTCTGTGGGCTGGGCCGTGATCACCGACGAGTACAAGG |
| | | TGCCCAGCAAGAAATTCAAGGTGCTGGGCAACACCGACCGGCACA |
| | | GCATCAAGAAGAACCTGATCGGAGCCCTGCTGTTCGACAGCGGCG |
| | | AAACAGCCGAGGCCACCCGGCTGAAGAGAACCGCCAGAAGAAGA |
| | | TACACCAGACGGAAGAACCGGATCTGCTATCTGCAAGAGATCTTC |
| | | AGCAACGAGATGGCCAAGGTGGACGACAGCTTCTTCCACAGACTG |
| | | GAAGAGTCCTTCCTGGTGGAAGAGGATAAGAAGCACGAGCGGCA |
| | | CCCCATCTTCGGCAACATCGTGGACGAGGTGGCCTACCACGAGAA |
| | | GTACCCCACCATCTACCACCTGAGAAAGAAACTGGTGGACAGCAC |
| | | CGACAAGGCCGACCTGCGGCTGATCTATCTGGCCCTGGCCCACAT |
| | | GATCAAGTTCCGGGGCCACTTCCTGATCGAGGGCGACCTGAACCC |
| | | CGACAACAGCGACGTGGACAAGCTGTTCATCCAGCTGGTGCAGAC |
| | | CTACAACCAGCTGTTCGAGGAAAACCCCATCAACGCCAGCGGCGT |
| | | GGACGCCAAGGCCATCCTGTCTGCCAGACTGAGCAAGAGCAGACG |
| | | GCTGGAAAATCTGATCGCCCAGCTGCCCGGCGAGAAGAAGAATG |
| | | GCCTGTTCGGCAACCTGATTGCCCTGAGCCTGGGCCTGACCCCCAA |
| | | CTTCAAGAGCAACTTCGACCTGGCCGAGGATGCCAAACTGCAGCT |
| | | GAGCAAGGACACCTACGACGACGACCTGGACAACCTGCTGGCCCA |
| | | GATCGGCGACCAGTACGCCGACCTGTTTCTGGCCGCCAAGAACCT |
| | | GTCCGACGCCATCCTGCTGAGCGACATCCTGAGAGTGAACACCGA |
| | | GATCACCAAGGCCCCCCTGAGCGCCTCTATGATCAAGAGATACGA |
| | | CGAGCACCACCAGGACCTGACCCTGCTGAAAGCTCTCGTGCGGCA |
| | | GCAGCTGCCTGAGAAGTACAAAGAGATTTTCTTCGACCAGAGCAA |
| | | GAACGGCTACGCCGGCTACATTGACGGCGGAGCCAGCCAGGAAG |
| | | AGTTCTACAAGTTCATCAAGCCCATCCTGGAAAAGATGGACGGCA |
| | | CCGAGGAACTGCTCGTGAAGCTGAACAGAGAGGACCTGCTGCGG |
| | | AAGCAGCGGACCTTCGACAACGGCAGCATCCCCCACCAGATCCAC |
| | | CTGGGAGAGCTGCACGCCATTCTGCGGCGGCAGGAAGATTTTTAC |
| | | CCATTCCTGAAGGACAACCGGGAAAAGATCGAGAAGATCCTGACC |
| | | TTCCGCATCCCCTACTACGTGGGCCCTCTGGCCAGGGGAAACAGC |
| | | AGATTCGCCTGGATGACCAGAAAGAGCGAGGAAACCATCACCCCC |
| | | TGGAACTTCGAGGAAGTGGTGGACAAGGGCGCTTCCGCCCAGAG |
| | | CTTCATCGAGCGGATGACCAACTTCGATAAGAACCTGCCCAACGA |
| | | GAAGGTGCTGCCCAAGCACAGCCTGCTGTACGAGTACTTCACCGT |
| | | GTATAACGAGCTGACCAAAGTGAAATACGTGACCGAGGGAATGA |
| | | GAAAGCCCGCCTTCCTGAGCGGCGAGCAGAAAAAGGCCATCGTG |
| | | GACCTGCTGTTCAAGACCAACCGGAAAGTGACCGTGAAGCAGCTG |
| | | AAAGAGGACTACTTCAAGAAAATCGAGTGCTTCGACTCCGTGGAA |
| | | ATCTCCGGCGTGGAAGATCGGTTCAACGCCTCCCTGGGCACATACC |
| | | ACGATCTGCTGAAAATTATCAAGGACAAGGACTTCCTGGACAATG |
| | | AGGAAAACGAGGACATTCTGGAAGATATCGTGCTGACCCTGACAC |
| | | TGTTTGAGGACAGAGAGATGATCGAGGAACGGCTGAAAACCTAT |
| | | GCCCACCTGTTCGACGACAAAGTGATGAAGCAGCTGAAGCGGCG |
| | | GAGATACACCGGCTGGGGCAGGCTGAGCCGGAAGCTGATCAACG |
| | | GCATCCGGGACAAGCAGTCCGGCAAGACAATCCTGGATTTCCTGA |
| | | AGTCCGACGGCTTCGCCAACAGAAACTTCATGCAGCTGATCCACG |
| | | ACGACAGCCTGACCTTTAAAGAGGACATCCAGAAAGCCCAGGTGT |
| | | CCGGCCAGGGCGATAGCCTGCACGAGCACATTGCCAATCTGGCCG |
| | | GCAGCCCCGCCATTAAGAAGGGCATCCTGCAGACAGTGAAGGTG |
| | | GTGGACGAGCTCGTGAAAGTGATGGGCCGGCACAAGCCCGAGAA |
| | | CATCGTGATCGAAATGGCCAGAGAGAACCAGACCACCCAGAAGG |
| | | GACAGAAGAACAGCCGCGAGAGAATGAAGCGGATCGAAGAGGG |
| | | CATCAAAGAGCTGGGCAGCCAGATCCTGAAAGAACACCCCGTGGA |
| | | AAACACCCAGCTGCAGAACGAGAAGCTGTACCTGTACTACCTGCA |
| | | GAATGGGCGGGATATGTACGTGGACCAGGAACTGGACATCAACC |
| | | GGCTGTCCGACTACGATGTGGACGCCATCGTGCCTCAGAGCTTTCT |
| | | GAAGGACGACTCCATCGACAACAAGGTGCTGACCAGAAGCGACA |
| | | AGAACCGGGGCAAGAGCGACAACGTGCCCTCCGAAGAGGTCGTG |
| | | AAGAAGATGAAGAACTACTGGCGGCAGCTGCTGAACGCCAAGCT |
| | | GATTACCCAGAGAAAGTTCGACAATCTGACCAAGGCCGAGAGAGG |
| | | CGGCCTGAGCGAACTGGATAAGGCCGGCTTCATCAAGAGACAGCT |
| | | GGTGGAAACCCGGCAGATCACAAAGCACGTGGCACAGATCCTGG |
| | | ACTCCCGGATGAACACTAAGTACGACGAGAATGACAAGCTGATCC |
| | | GGGAAGTGAAAGTGATCACCCTGAAGTCCAAGCTGGTGTCCGATT |
| | | TCCGGAAGGATTTCCAGTTTTACAAAGTGCGCGAGATCAACAACTA |
| | | CCACCACGCCCACGACGCCTACCTGAACGCCGTCGTGGGAACCGC |
| | | CCTGATCAAAAAGTACCCTAAGCTGGAAAGCGAGTTCGTGTACGG |
| | | CGACTACAAGGTGTACGACGTGCGGAAGATGATCGCCAAGAGCG |
| | | AGCAGGAAATCGGCAAGGCTACCGCCAAGTACTTCTTCTACAGCA |
| | | ACATCATGAACTTTTTCAAGACCGAGATTACCCTGGCCAACGGCGA |
| | | GATCCGGAAGCGGCCTCTGATCGAGACAAACGGCGAAACCGGGG |
| | | AGATCGTGTGGGATAAGGGCCGGGATTTTGCCACCGTGCGGAAA |
| | | GTGCTGAGCATGCCCCAAGTGAATATCGTGAAAAAGACCGAGGTG |
| | | CAGACAGGCGGCTTCAGCAAAGAGTCTATCCTGCCCAAGAGGAAC |
| | | AGCGATAAGCTGATCGCCAGAAAGAAGGACTGGGACCCTAAGAA |
| | | GTACGGCGGCTTCGACAGCCCCACCGTGGCCTATTCTGTGCTGGTG |
| | | GTGGCCAAAGTGGAAAAGGGCAAGTCCAAGAAACTGAAGAGTGT |
| | | GAAAGAGCTGCTGGGGATCACCATCATGGAAAGAAGCAGCTTCGA |
| | | GAAGAATCCCATCGACTTTCTGGAAGCCAAGGGCTACAAAGAAGT |
| | | GAAAAAGGACCTGATCATCAAGCTGCCTAAGTACTCCCTGTTCGAG |
| | | CTGGAAAACGGCCGGAAGAGAATGCTGGCCTCTGCCGGCGAACT |
| | | GCAGAAGGGAAACGAACTGGCCCTGCCCTCCAAATATGTGAACTT |
| | | CCTGTACCTGGCCAGCCACTATGAGAAGCTGAAGGGCTCCCCCGA |
| | | GGATAATGAGCAGAAACAGCTGTTTGTGGAACAGCACAAGCACTA |
| | | CCTGGACGAGATCATCGAGCAGATCAGCGAGTTCTCCAAGAGAGT |
| | | GATCCTGGCCGACGCTAATCTGGACAAAGTGCTGTCCGCCTACAAC |
| | | AAGCACCGGGATAAGCCCATCAGAGAGCAGGCCGAGAATATCATC |
| | | CACCTGTTTACCCTGACCAATCTGGGAGCCCCTGCCGCCTTCAAGT |
| | | ACTTTGACACCACCATCGACCGGAAGAGGTACACCAGCACCAAAG |
| | | AGGTGCTGGACGCCACCCTGATCCACCAGAGCATCACCGGCCTGT |
| | | ACGAGACACGGATCGACCTGTCTCAGCTGGGAGGCGACAGCCCCA |
| | | AGAAGAAGAGAAAGGTGGGAGTCGACGGATCCAGCGGCTCCGAG |
| | | ACCCCAGGCACATCTGAGAGCGCCACCCCTGAGTCCACCGGTGAC |
| | | TCCGTTGCTTTCGAGGACGTGGCCGTGAACTTCACACTTGAGGAAT |
| | | GGGCCTTGCTCGACCCAAGTCAGAAGAATCTGTACAGAGACGTGA |
| | | TGCGGGAGACATTCAGGAATCTCGCCAGTGTCGGAAAGCAGTGG |
| | | GAAGACCAGAACATCGAAGATCCTTTCAAGATACCACGGCGCAAT |
| | | ATCTCCCACATTCCTGAGAGGCTGTGTGAATCTAAGGAAGGCGGA |
| | | CAAGGTGAGGAAAGCGCTGATTACAAAGATGATGACGATAAAGC |
| | | CCCCAAGAAGAAAAGGAAGGTCCCAAAGAAAAAAAGAAAGGTGT |
| | | GA |
| | Fusion Protein | MYPYDVPDYASPKKKRKVNHDQEFDPPKVYPPVPAEKRKPIRVLSLFD |
| | 13 Amino Acid | GIATGLLVLKDLGIQVDRYIASEVCEDSITVGMVRHQGKIMYVGDVRS |
| | Sequence | VTQKHIQEWGPFDLVIGGSPCNDLSIVNPARKGLYEGTGRLFFEFYRLL |
| | NLS-3A-ADD- | HDARPKEGDDRPFFWLFENVVAMGVSDKRDISRFLESNPVMIDAKE |
| | h3L-dCas9-NLS- | VSAAHRARYFWGNLPGMNRPLASTVNDKLELQECLEHGRIAKFSKVR |
| | KOX1KRAB | TITTRSNSIKQGKDQHFPVFMNEKEDILWCTEMERVFGFPVHYTDVS |
| | | NMSRLARQRLLGRSWSVPVIRHLFAPLKEYFACVSSGNSNANSRGPS |
| | | FSSGLVPLSLRGSHMAAIPALDPEAEPSMDVILVGSSELSSSVSPGTGR |
| | | DLIAYEVKANQRNIEDICICCGSLQVHTQHPLFEGGICAPCKDKFLDAL |
| | | FLYDDDGYQSYCSICCSGETLLICGNPDCTRCYCFECVDSLVGPGTSGK |
| | | VHAMSNWVCYLCLPSSRSGLLQRRRKWRSQLKAFYDRESENPLEMF |
| | | ETVPVWRRQPVRVLSLFEDIKKELTSLGFLESGSDPGQLKHVVDVTDT |
| | | VRKDVEEWGPFDLVYGATPPLGHTCDRPPSWYLFQFHRLLQYARPKP |
| | | GSPRPFFWMFVDNLVLNKEDLDVASRFLEMEPVTIPDVHGGSLQNA |
| | | VRVWSNIPAIRSRHWALVSEEELSLLAQNKQSSKLAAKWPTKLVKNCF |
| | | LPLREYFKYFSTELTSSLGGPSSGAPPPSGGSPAGSPTSTEEGTSESATP |
| | | ESGPGTSTEPSEGSAPGSPAGSPTSTEEGTSTEPSEGSAPGTSTEPSELE |
| | | DKKYSIGLAIGTNSVGWAVITDEYKVPSKKFKVLGNTDRHSIKKNLIGA |
| | | LLFDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTDKA |
| | | DLRLIYLALAHMIKFRGHFLIEGDLNPDNSDVDKLFIQLVQTYNQLFEE |
| | | NPINASGVDAKAILSARLSKSRRLENLIAQLPGEKKNGLFGNLIALSLGL |
| | | TPNFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLPEK |
| | | YKEIFFDQSKNGYAGYIDGGASQEEFYKFIKPILEKMDGTEELLVKLNRE |
| | | DLLRKQRTFDNGSIPHQIHLGELHAILRRQEDFYPFLKDNREKIEKILTF |
| | | RIPYYVGPLARGNSRFAWMTRKSEETITPWNFEEVVDKGASAQSFIER |
| | | MTNFDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFNASLG |
| | | TYHDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKGIL |
| | | QTVKVVDELVKVMGRHKPENIVIEMARENQTTQKGQKNSRERMKRI |
| | | EEGIKELGSQILKEHPVENTQLQNEKLYLYYLQNGRDMYVDQELDINR |
| | | LSDYDVDAIVPQSFLKDDSIDNKVLTRSDKNRGKSDNVPSEEVVKKMK |
| | | NYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVRE |
| | | INNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWD |
| | | KGRDFATVRKVLSMPQVNIVKKTEVQTGGFSKESILPKRNSDKLIARKK |
| | | DWDPKKYGGFDSPTVAYSVLVVAKVEKGKSKKLKSVKELLGITIMERS |
| | | SFEKNPIDFLEAKGYKEVKKDLIIKLPKYSLFELENGRKRMLASAGELQK |
| | | GNELALPSKYVNFLYLASHYEKLKGSPEDNEQKQLFVEQHKHYLDEIIE |
| | | QISEFSKRVILADANLDKVLSAYNKHRDKPIREQAENIIHLFTLTNLGAP |
| | | AAFKYFDTTIDRKRYTSTKEVLDATLIHQSITGLYETRIDLSQLGGDSPKK |
| | | KRKVGVDGSSGSETPGTSESATPESRTLVTFKDVFVDFTREEWKLLDT |
| | | AQQIVYRNVMLENYKNLVSLGYQLTKPDVILRLEKGEEPWLV |
| | Fusion Protein | ATGTACCCATACGATGTTCCAGATTACGCTTCGCCGAAGAAAAAGC |
| | 13 DNA | GCAAGGTCAATCACGATCAGGAGTTCGACCCCCCTAAGGTGTACC |
| | Sequence | CACCAGTGCCTGCAGAGAAGAGGAAGCCAATCCGGGTGCTGAGC |
| | | CTGTTTGATGGCATCGCCACCGGCCTGCTGGTGCTGAAGGATCTG |
| | | GGCATCCAGGTGGACCGGTACATCGCCTCCGAGGTGTGCGAGGAT |
| | | TCTATCACCGTGGGCATGGTGCGCCACCAGGGCAAGATCATGTAT |
| | | GTGGGCGACGTGCGGTCCGTGACACAGAAGCACATCCAGGAGTG |
| | | GGGCCCATTCGATCTGGTGATCGGCGGCAGCCCCTGTAATGACCT |
| | | GTCCATCGTGAACCCTGCAAGGAAGGGACTGTACGAGGGAACCG |
| | | GCCGGCTGTTCTTTGAGTTTTATAGACTGCTGCACGACGCCAGGCC |
| | | TAAGGAGGGCGACGATAGACCATTCTTTTGGCTGTTCGAGAATGT |
| | | GGTGGCTATGGGCGTGAGCGATAAGAGGGACATCTCCAGGTTTCT |
| | | GGAGTCTAACCCCGTGATGATCGATGCAAAGGAGGTGTCCGCCGC |
| | | ACACAGAGCCAGGTATTTCTGGGGCAATCTGCCAGGAATGAACAG |
| | | GCCACTGGCAAGCACCGTGAATGACAAGCTGGAGCTGCAGGAGT |
| | | GCCTGGAGCACGGAAGGATCGCCAAGTTTTCCAAGGTGCGCACAA |
| | | TCACCACACGGAGCAATTCCATCAAGCAGGGCAAGGATCAGCACT |
| | | TCCCCGTGTTCATGAACGAGAAGGAGGACATCCTGTGGTGTACCG |
| | | AGATGGAGAGAGTGTTCGGCTTTCCAGTGCACTACACAGACGTGT |
| | | CTAACATGAGCAGGCTGGCAAGGCAGCGGCTGCTGGGCAGATCTT |
| | | GGAGCGTGCCCGTGATCAGGCACCTGTTCGCCCCTCTGAAGGAGT |
| | | ATTTTGCCTGCGTGAGCAGCGGCAACTCCAATGCCAACAGCCGGG |
| | | GCCCCTCTTTCAGCTCCGGATTGGTGCCTCTGAGCCTGAGGGGCTC |
| | | CCACATGGCAGCAATCCCCGCCCTGGACCCCGAGGCCGAGCCTAG |
| | | CATGGACGTGATCCTGGTGGGCTCTAGCGAGCTGTCCTCTAGCGT |
| | | GTCTCCAGGAACCGGAAGGGATCTGATCGCATACGAGGTGAAGG |
| | | CCAATCAGCGGAACATCGAGGACATCTGTATCTGCTGTGGCAGCC |
| | | TGCAGGTGCACACACAGCACCCACTGTTCGAGGGAGGAATCTGCG |
| | | CACCCTGTAAGGATAAGTTCCTGGACGCCCTGTTTCTGTACGACGA |
| | | TGACGGCTACCAGTCCTATTGCTCTATCTGCTGTTCCGGCGAGACC |
| | | CTGCTGATCTGCGGCAATCCAGATTGTACAAGGTGCTATTGTTTTG |
| | | AGTGCGTGGACTCTCTGGTGGGACCAGGCACCAGCGGAAAGGTG |
| | | CACGCCATGTCCAACTGGGTGTGCTACCTGTGCCTGCCATCCTCTC |
| | | GCAGCGGACTGCTGCAGCGGAGAAGGAAGTGGAGATCCCAGCTG |
| | | AAGGCCTTCTATGATAGGGAGTCTGAGAACCCCCTGGAGATGTTT |
| | | GAGACCGTGCCAGTGTGGCGCCGGCAGCCCGTGAGGGTGCTGAG |
| | | CCTGTTCGAGGATATCAAGAAGGAGCTGACATCCCTGGGCTTTCTG |
| | | GAGTCCGGCTCTGACCCCGGACAGCTGAAGCACGTGGTGGATGTG |
| | | ACCGACACAGTGCGGAAGGATGTGGAGGAGTGGGGCCCTTTCGA |
| | | CCTGGTGTACGGAGCAACCCCTCCACTGGGACACACATGCGACAG |
| | | ACCCCCTTCTTGGTACCTGTTCCAGTTTCACCGCCTGCTGCAGTATG |
| | | CAAGGCCAAAGCCAGGCAGCCCTAGACCATTCTTTTGGATGTTCGT |
| | | GGATAATCTGGTGCTGAACAAGGAGGATCTGGACGTGGCCAGCA |
| | | GGTTTCTGGAGATGGAGCCAGTGACCATCCCAGACGTGCACGGCG |
| | | GCTCCCTGCAGAATGCCGTGCGCGTGTGGTCTAACATCCCTGCCAT |
| | | CAGAAGCAGGCACTGGGCACTGGTGAGCGAGGAGGAGCTGTCCC |
| | | TGCTGGCCCAGAATAAGCAGAGCAGCAAGCTGGCCGCCAAGTGG |
| | | CCTACAAAGCTGGTGAAGAACTGCTTCCTGCCACTGCGGGAGTAC |
| | | TTCAAGTATTTTTCCACCGAGCTGACATCTAGCCTGGGAGGACCCT |
| | | CCTCTGGCGCCCCACCACCTAGCGGCGGCTCCCCTGCCGGCTCTCC |
| | | AACCAGCACAGAGGAGGGCACCAGCGAGTCCGCCACACCAGAGT |
| | | CTGGACCTGGCACCAGCACAGAGCCATCCGAGGGCTCTGCCCCAG |
| | | GCTCTCCTGCAGGCAGCCCTACCTCCACCGAAGAGGGCACCAGCA |
| | | CAGAGCCTTCTGAGGGCAGCGCCCCAGGCACCTCTACAGAGCCAA |
| | | GCGAGCTCGAGGACAAGAAGTACAGCATCGGCCTGGCCATCGGC |
| | | ACCAACTCTGTGGGCTGGGCCGTGATCACCGACGAGTACAAGGTG |
| | | CCCAGCAAGAAATTCAAGGTGCTGGGCAACACCGACCGGCACAGC |
| | | ATCAAGAAGAACCTGATCGGAGCCCTGCTGTTCGACAGCGGCGAA |
| | | ACAGCCGAGGCCACCCGGCTGAAGAGAACCGCCAGAAGAAGATA |
| | | CACCAGACGGAAGAACCGGATCTGCTATCTGCAAGAGATCTTCAG |
| | | CAACGAGATGGCCAAGGTGGACGACAGCTTCTTCCACAGACTGGA |
| | | AGAGTCCTTCCTGGTGGAAGAGGATAAGAAGCACGAGCGGCACC |
| | | CCATCTTCGGCAACATCGTGGACGAGGTGGCCTACCACGAGAAGT |
| | | ACCCCACCATCTACCACCTGAGAAAGAAACTGGTGGACAGCACCG |
| | | ACAAGGCCGACCTGCGGCTGATCTATCTGGCCCTGGCCCACATGAT |
| | | CAAGTTCCGGGGCCACTTCCTGATCGAGGGCGACCTGAACCCCGA |
| | | CAACAGCGACGTGGACAAGCTGTTCATCCAGCTGGTGCAGACCTA |
| | | CAACCAGCTGTTCGAGGAAAACCCCATCAACGCCAGCGGCGTGGA |
| | | CGCCAAGGCCATCCTGTCTGCCAGACTGAGCAAGAGCAGACGGCT |
| | | GGAAAATCTGATCGCCCAGCTGCCCGGCGAGAAGAAGAATGGCCT |
| | | GTTCGGCAACCTGATTGCCCTGAGCCTGGGCCTGACCCCCAACTTC |
| | | AAGAGCAACTTCGACCTGGCCGAGGATGCCAAACTGCAGCTGAGC |
| | | AAGGACACCTACGACGACGACCTGGACAACCTGCTGGCCCAGATC |
| | | GGCGACCAGTACGCCGACCTGTTTCTGGCCGCCAAGAACCTGTCC |
| | | GACGCCATCCTGCTGAGCGACATCCTGAGAGTGAACACCGAGATC |
| | | ACCAAGGCCCCCCTGAGCGCCTCTATGATCAAGAGATACGACGAG |
| | | CACCACCAGGACCTGACCCTGCTGAAAGCTCTCGTGCGGCAGCAG |
| | | CTGCCTGAGAAGTACAAAGAGATTTTCTTCGACCAGAGCAAGAAC |
| | | GGCTACGCCGGCTACATTGACGGCGGAGCCAGCCAGGAAGAGTT |
| | | CTACAAGTTCATCAAGCCCATCCTGGAAAAGATGGACGGCACCGA |
| | | GGAACTGCTCGTGAAGCTGAACAGAGAGGACCTGCTGCGGAAGC |
| | | AGCGGACCTTCGACAACGGCAGCATCCCCCACCAGATCCACCTGG |
| | | GAGAGCTGCACGCCATTCTGCGGCGGCAGGAAGATTTTTACCCAT |
| | | TCCTGAAGGACAACCGGGAAAAGATCGAGAAGATCCTGACCTTCC |
| | | GCATCCCCTACTACGTGGGCCCTCTGGCCAGGGGAAACAGCAGAT |
| | | TCGCCTGGATGACCAGAAAGAGCGAGGAAACCATCACCCCCTGGA |
| | | ACTTCGAGGAAGTGGTGGACAAGGGCGCTTCCGCCCAGAGCTTCA |
| | | TCGAGCGGATGACCAACTTCGATAAGAACCTGCCCAACGAGAAGG |
| | | TGCTGCCCAAGCACAGCCTGCTGTACGAGTACTTCACCGTGTATAA |
| | | CGAGCTGACCAAAGTGAAATACGTGACCGAGGGAATGAGAAAGC |
| | | CCGCCTTCCTGAGCGGCGAGCAGAAAAAGGCCATCGTGGACCTGC |
| | | TGTTCAAGACCAACCGGAAAGTGACCGTGAAGCAGCTGAAAGAG |
| | | GACTACTTCAAGAAAATCGAGTGCTTCGACTCCGTGGAAATCTCCG |
| | | GCGTGGAAGATCGGTTCAACGCCTCCCTGGGCACATACCACGATC |
| | | TGCTGAAAATTATCAAGGACAAGGACTTCCTGGACAATGAGGAAA |
| | | ACGAGGACATTCTGGAAGATATCGTGCTGACCCTGACACTGTTTGA |
| | | GGACAGAGAGATGATCGAGGAACGGCTGAAAACCTATGCCCACCT |
| | | GTTCGACGACAAAGTGATGAAGCAGCTGAAGCGGCGGAGATACA |
| | | CCGGCTGGGGCAGGCTGAGCCGGAAGCTGATCAACGGCATCCGG |
| | | GACAAGCAGTCCGGCAAGACAATCCTGGATTTCCTGAAGTCCGAC |
| | | GGCTTCGCCAACAGAAACTTCATGCAGCTGATCCACGACGACAGC |
| | | CTGACCTTTAAAGAGGACATCCAGAAAGCCCAGGTGTCCGGCCAG |
| | | GGCGATAGCCTGCACGAGCACATTGCCAATCTGGCCGGCAGCCCC |
| | | GCCATTAAGAAGGGCATCCTGCAGACAGTGAAGGTGGTGGACGA |
| | | GCTCGTGAAAGTGATGGGCCGGCACAAGCCCGAGAACATCGTGAT |
| | | CGAAATGGCCAGAGAGAACCAGACCACCCAGAAGGGACAGAAGA |
| | | ACAGCCGCGAGAGAATGAAGCGGATCGAAGAGGGCATCAAAGAG |
| | | CTGGGCAGCCAGATCCTGAAAGAACACCCCGTGGAAAACACCCAG |
| | | CTGCAGAACGAGAAGCTGTACCTGTACTACCTGCAGAATGGGCGG |
| | | GATATGTACGTGGACCAGGAACTGGACATCAACCGGCTGTCCGAC |
| | | TACGATGTGGACGCCATCGTGCCTCAGAGCTTTCTGAAGGACGAC |
| | | TCCATCGACAACAAGGTGCTGACCAGAAGCGACAAGAACCGGGG |
| | | CAAGAGCGACAACGTGCCCTCCGAAGAGGTCGTGAAGAAGATGA |
| | | AGAACTACTGGCGGCAGCTGCTGAACGCCAAGCTGATTACCCAGA |
| | | GAAAGTTCGACAATCTGACCAAGGCCGAGAGAGGCGGCCTGAGC |
| | | GAACTGGATAAGGCCGGCTTCATCAAGAGACAGCTGGTGGAAACC |
| | | CGGCAGATCACAAAGCACGTGGCACAGATCCTGGACTCCCGGATG |
| | | AACACTAAGTACGACGAGAATGACAAGCTGATCCGGGAAGTGAA |
| | | AGTGATCACCCTGAAGTCCAAGCTGGTGTCCGATTTCCGGAAGGA |
| | | TTTCCAGTTTTACAAAGTGCGCGAGATCAACAACTACCACCACGCC |
| | | CACGACGCCTACCTGAACGCCGTCGTGGGAACCGCCCTGATCAAA |
| | | AAGTACCCTAAGCTGGAAAGCGAGTTCGTGTACGGCGACTACAAG |
| | | GTGTACGACGTGCGGAAGATGATCGCCAAGAGCGAGCAGGAAAT |
| | | CGGCAAGGCTACCGCCAAGTACTTCTTCTACAGCAACATCATGAAC |
| | | TTTTTCAAGACCGAGATTACCCTGGCCAACGGCGAGATCCGGAAG |
| | | CGGCCTCTGATCGAGACAAACGGCGAAACCGGGGAGATCGTGTG |
| | | GGATAAGGGCCGGGATTTTGCCACCGTGCGGAAAGTGCTGAGCAT |
| | | GCCCCAAGTGAATATCGTGAAAAAGACCGAGGTGCAGACAGGCG |
| | | GCTTCAGCAAAGAGTCTATCCTGCCCAAGAGGAACAGCGATAAGC |
| | | TGATCGCCAGAAAGAAGGACTGGGACCCTAAGAAGTACGGCGGC |
| | | TTCGACAGCCCCACCGTGGCCTATTCTGTGCTGGTGGTGGCCAAAG |
| | | TGGAAAAGGGCAAGTCCAAGAAACTGAAGAGTGTGAAAGAGCTG |
| | | CTGGGGATCACCATCATGGAAAGAAGCAGCTTCGAGAAGAATCCC |
| | | ATCGACTTTCTGGAAGCCAAGGGCTACAAAGAAGTGAAAAAGGAC |
| | | CTGATCATCAAGCTGCCTAAGTACTCCCTGTTCGAGCTGGAAAACG |
| | | GCCGGAAGAGAATGCTGGCCTCTGCCGGCGAACTGCAGAAGGGA |
| | | AACGAACTGGCCCTGCCCTCCAAATATGTGAACTTCCTGTACCTGG |
| | | CCAGCCACTATGAGAAGCTGAAGGGCTCCCCCGAGGATAATGAGC |
| | | AGAAACAGCTGTTTGTGGAACAGCACAAGCACTACCTGGACGAGA |
| | | TCATCGAGCAGATCAGCGAGTTCTCCAAGAGAGTGATCCTGGCCG |
| | | ACGCTAATCTGGACAAAGTGCTGTCCGCCTACAACAAGCACCGGG |
| | | ATAAGCCCATCAGAGAGCAGGCCGAGAATATCATCCACCTGTTTAC |
| | | CCTGACCAATCTGGGAGCCCCTGCCGCCTTCAAGTACTTTGACACC |
| | | ACCATCGACCGGAAGAGGTACACCAGCACCAAAGAGGTGCTGGA |
| | | CGCCACCCTGATCCACCAGAGCATCACCGGCCTGTACGAGACACG |
| | | GATCGACCTGTCTCAGCTGGGAGGCGACAGCCCCAAGAAGAAGA |
| | | GAAAGGTGGGAGTCGACGGATCCAGCGGCTCCGAGACCCCAGGC |
| | | ACATCTGAGAGCGCCACCCCTGAGTCCCGGACCCTGGTGACATTCA |
| | | AGGACGTGTTCGTGGACTTCACCCGGGAGGAGTGGAAGCTGCTG |
| | | GACACAGCCCAGCAGATCGTGTACAGGAACGTGATGCTGGAGAAC |
| | | TATAAGAATCTGGTGTCTCTGGGCTACCAGCTGACAAAGCCAGAT |
| | | GTGATCCTGCGGCTGGAGAAGGGAGAGGAGCCCTGGCTGGTGTA |
| | | G |
| | Fusion Protein | MGTMNHDQEFDPPKVYPPVPAEKRKPIRVLSLFDGIATGLLVLKDLGI |
| | 14 Amino Acid | QVDRYIASEVCEDSITVGMVRHQGKIMYVGDVRSVTQKHIQEWGPF |
| | Sequence | DLVIGGSPCNDLSIVNPARKGLYEGTGRLFFEFYRLLHDARPKEGDDRP |
| | 3A-3L-NLS- | FFWLFENVVAMGVSDKRDISRFLESNPVMIDAKEVSAAHRARYFWG |
| | dCas9-NLS- | NLPGMNRPLASTVNDKLELQECLEHGRIAKFSKVRTITTRSNSIKQGK |
| | KOX1KRAB | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGSH |
| | | MGPMEIYKTVSAWKRQPVRVLSLFRNIDKVLKSLGFLESGSGSGGGTL |
| | | KYVEDVTNVVRRDVEKWGPFDLVYGSTQPLGSSCDRCPGWYMFQF |
| | | HRILQYALPRQESQRPFFWIFMDNLLLTEDDQETTTRFLQTEAVTLQD |
| | | VRGRDYQNAMRVWSNIPGLKSKHAPLTPKEEEYLQAQVRSRSKLDA |
| | | PKVDLLVKNCLLPLREYFKYFSQNSLPLGGPSSGAPPPSGGSPAGSPTS |
| | | TEEGTSESATPESGPGTSTEPSEGSAPGSPAGSPTSTEEGTSTEPSEGS |
| | | APGTSTEPSEPKKKRKVYMDKKYSIGLAIGTNSVGWAVITDEYKVPSK |
| | | KFKVLGNTDRHSIKKNLIGALLFDSGETAEATRLKRTARRRYTRRKNRIC |
| | | YLQEIFSNEMAKVDDSFFHRLEESFLVEEDKKHERHPIFGNIVDEVAYH |
| | | EKYPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIAQL |
| | | PGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDE |
| | | HHQDLTLLKALVRQQLPEKYKEIFFDQSKNGYAGYIDGGASQEEFYKFI |
| | | KPILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETITP |
| | | WNFEEVVDKGASAQSFIERMTNFDKNLPNEKVLPKHSLLYEYFTVYN |
| | | ELTKVKYVTEGMRKPAFLSGEQKKAIVDLLFKTNRKVTVKQLKEDYFKK |
| | | IECFDSVEISGVEDRFNASLGTYHDLLKIIKDKDFLDNEENEDILEDIVLT |
| | | LTLFEDREMIEERLKTYAHLFDDKVMKQLKRRRYTGWGRLSRKLINGI |
| | | RDKQSGKTILDFLKSDGFANRNFMQLIHDDSLTFKEDIQKAQVSGQG |
| | | DSLHEHIANLAGSPAIKKGILQTVKVVDELVKVMGRHKPENIVIEMAR |
| | | ENQTTQKGQKNSRERMKRIEEGIKELGSQILKEHPVENTQLQNEKLYL |
| | | YYLQNGRDMYVDQELDINRLSDYDVDAIVPQSFLKDDSIDNKVLTRSD |
| | | KNRGKSDNVPSEEVVKKMKNYWRQLLNAKLITQRKFDNLTKAERGG |
| | | LSELDKAGFIKRQLVETRQITKHVAQILDSRMNTKYDENDKLIREVKVI |
| | | TLKSKLVSDFRKDFQFYKVREINNYHHAHDAYLNAVVGTALIKKYPKLE |
| | | SEFVYGDYKVYDVRKMIAKSEQEIGKATAKYFFYSNIMNFFKTEITLAN |
| | | GEIRKRPLIETNGETGEIVWDKGRDFATVRKVLSMPQVNIVKKTEVQT |
| | | GGFSKESILPKRNSDKLIARKKDWDPKKYGGFDSPTVAYSVLVVAKVE |
| | | KGKSKKLKSVKELLGITIMERSSFEKNPIDFLEAKGYKEVKKDLIIKLPKYS |
| | | LFELENGRKRMLASAGELQKGNELALPSKYVNFLYLASHYEKLKGSPE |
| | | DNEQKQLFVEQHKHYLDEIIEQISEFSKRVILADANLDKVLSAYNKHRD |
| | | KPIREQAENIIHLFTLTNLGAPAAFKYFDTTIDRKRYTSTKEVLDATLIHQ |
| | | SITGLYETRIDLSQLGGDPKKKRKVSGSETPGTSESATPESTGRTLVTFK |
| | | DVFVDFTREEWKLLDTAQQIVYRNVMLENYKNLVSLGYQLTKPDVILR |
| | | LEKGEEP |
| | Fusion Protein | ATGGGTACCATGAACCATGACCAGGAATTTGACCCCCCAAAGGTTT |
| | 14 DNA | ACCCACCTGTGCCAGCTGAGAAGAGGAAGCCCATCCGCGTGCTGT |
| | Sequence | CTCTCTTTGATGGGATTGCTACAGGGCTCCTGGTGCTGAAGGACCT |
| | | GGGCATCCAAGTGGACCGCTACATTGCCTCCGAGGTGTGTGAGGA |
| | | CTCCATCACGGTGGGCATGGTGCGGCACCAGGGAAAGATCATGTA |
| | | CGTCGGGGACGTCCGCAGCGTCACACAGAAGCATATCCAGGAGTG |
| | | GGGCCCATTCGACCTGGTGATTGGAGGCAGTCCCTGCAATGACCT |
| | | CTCCATTGTCAACCCTGCCCGCAAGGGACTTTATGAGGGTACTGGC |
| | | CGCCTCTTCTTTGAGTTCTACCGCCTCCTGCATGATGCGCGGCCCAA |
| | | GGAGGGAGATGATCGCCCCTTCTTCTGGCTCTTTGAGAATGTGGT |
| | | GGCCATGGGCGTTAGTGACAAGAGGGACATCTCGCGATTTCTTGA |
| | | GTCTAACCCCGTGATGATTGACGCCAAAGAAGTGTCTGCTGCACAC |
| | | AGGGCCCGTTACTTCTGGGGTAACCTTCCTGGCATGAACAGGCCTT |
| | | TGGCATCCACTGTGAATGATAAGCTGGAGCTGCAAGAGTGTCTGG |
| | | AGCACGGCAGAATAGCCAAGTTCAGCAAAGTGAGGACCATTACCA |
| | | CCAGGTCAAACTCTATAAAGCAGGGCAAAGACCAGCATTTCCCCGT |
| | | CTTCATGAACGAGAAGGAGGACATCCTGTGGTGCACTGAAATGGA |
| | | AAGGGTGTTTGGCTTCCCCGTCCACTACACAGACGTCTCCAACATG |
| | | AGCCGCTTGGCGAGGCAGAGACTGCTGGGCCGATCGTGGAGCGT |
| | | GCCGGTCATCCGCCACCTCTTCGCTCCGCTGAAGGAATATTTTGCTT |
| | | GTGTGTCTAGCGGCAATAGTAACGCTAACAGCCGCGGGCCGAGCT |
| | | TCAGCAGCGGCCTGGTGCCGTTAAGCTTGCGCGGCAGCCATATGG |
| | | GCCCTATGGAGATATACAAGACAGTGTCTGCATGGAAGAGACAGC |
| | | CAGTGCGGGTACTGAGCCTCTTCAGAAACATCGACAAGGTACTAA |
| | | AGAGTTTGGGCTTCTTGGAAAGCGGTTCTGGTTCTGGGGGAGGAA |
| | | CGCTGAAGTACGTGGAAGATGTCACAAATGTCGTGAGGAGAGAC |
| | | GTGGAGAAATGGGGCCCCTTTGACCTGGTGTACGGCTCGACGCAG |
| | | CCCCTAGGCAGCTCTTGTGATCGCTGTCCCGGCTGGTACATGTTCC |
| | | AGTTCCACCGGATCCTGCAGTATGCGCTGCCTCGCCAGGAGAGTC |
| | | AGCGGCCCTTCTTCTGGATATTCATGGACAATCTGCTGCTGACTGA |
| | | GGATGACCAAGAGACAACTACCCGCTTCCTTCAGACAGAGGCTGT |
| | | GACCCTCCAGGATGTCCGTGGCAGAGACTACCAGAATGCTATGCG |
| | | GGTGTGGAGCAACATTCCAGGGCTGAAGAGCAAGCATGCGCCCCT |
| | | GACCCCAAAGGAAGAAGAGTATCTGCAAGCCCAAGTCAGAAGCA |
| | | GGAGCAAGCTGGACGCCCCGAAAGTTGACCTCCTGGTGAAGAACT |
| | | GCCTTCTCCCGCTGAGAGAGTACTTCAAGTATTTTTCTCAAAACTCA |
| | | CTTCCTCTTGGAGGGCCGAGCTCTGGCGCACCCCCACCAAGTGGA |
| | | GGGTCTCCTGCCGGGTCCCCAACATCTACTGAAGAAGGCACCAGC |
| | | GAATCCGCAACGCCCGAGTCAGGCCCTGGTACCTCCACAGAACCA |
| | | TCTGAAGGTAGTGCGCCTGGTTCCCCAGCTGGAAGCCCTACTTCCA |
| | | CCGAAGAAGGCACGTCAACCGAACCAAGTGAAGGATCTGCCCCTG |
| | | GGACCAGCACTGAACCATCTGAGCCAAAAAAGAAGAGAAAGGTA |
| | | TACATGGACAAGAAGTACAGCATCGGCCTGGCCATCGGCACCAAC |
| | | TCTGTGGGCTGGGCCGTGATCACCGACGAGTACAAGGTGCCCAGC |
| | | AAGAAATTCAAGGTGCTGGGCAACACCGACCGGCACAGCATCAAG |
| | | AAGAACCTGATCGGCGCCCTGCTGTTCGACAGCGGAGAAACAGCC |
| | | GAGGCCACCCGGCTGAAGAGAACCGCCAGAAGAAGATACACCAG |
| | | ACGGAAGAACCGGATCTGCTATCTGCAAGAGATCTTCAGCAACGA |
| | | GATGGCCAAGGTGGACGACAGCTTCTTCCACAGACTGGAAGAGTC |
| | | CTTCCTGGTGGAAGAGGATAAGAAGCACGAGCGGCACCCCATCTT |
| | | CGGCAACATCGTGGACGAGGTGGCCTACCACGAGAAGTACCCCAC |
| | | CATCTACCACCTGAGAAAGAAACTGGTGGACAGCACCGACAAGGC |
| | | CGACCTGCGGCTGATCTATCTGGCCCTGGCCCACATGATCAAGTTC |
| | | CGGGGCCACTTCCTGATCGAGGGCGACCTGAACCCCGACAACAGC |
| | | GACGTGGACAAGCTGTTCATCCAGCTGGTGCAGACCTACAACCAG |
| | | CTGTTCGAGGAAAACCCCATCAACGCCAGCGGCGTGGACGCCAAG |
| | | GCCATCCTGTCTGCCAGACTGAGCAAGAGCAGACGGCTGGAAAAT |
| | | CTGATCGCCCAGCTGCCCGGCGAGAAGAAGAATGGCCTGTTCGGC |
| | | AACCTGATTGCCCTGAGCCTGGGCCTGACCCCCAACTTCAAGAGCA |
| | | ACTTCGACCTGGCCGAGGATGCCAAACTGCAGCTGAGCAAGGACA |
| | | CCTACGACGACGACCTGGACAACCTGCTGGCCCAGATCGGCGACC |
| | | AGTACGCCGACCTGTTTCTGGCCGCCAAGAACCTGTCCGACGCCAT |
| | | CCTGCTGAGCGACATCCTGAGAGTGAACACCGAGATCACCAAGGC |
| | | CCCCCTGAGCGCCTCTATGATCAAGAGATACGACGAGCACCACCA |
| | | GGACCTGACCCTGCTGAAAGCTCTCGTGCGGCAGCAGCTGCCTGA |
| | | GAAGTACAAAGAGATTTTCTTCGACCAGAGCAAGAACGGCTACGC |
| | | CGGCTACATCGATGGCGGAGCCAGCCAGGAAGAGTTCTACAAGTT |
| | | CATCAAGCCCATCCTGGAAAAGATGGACGGCACCGAGGAACTGCT |
| | | CGTGAAGCTGAACAGAGAGGACCTGCTGCGGAAGCAGCGGACCT |
| | | TCGACAACGGCAGCATCCCCCACCAGATCCACCTGGGAGAGCTGC |
| | | ACGCCATTCTGCGGCGGCAGGAAGATTTTTACCCATTCCTGAAGGA |
| | | CAACCGGGAAAAGATCGAGAAGATCCTGACCTTCCGCATCCCCTAC |
| | | TACGTGGGCCCTCTGGCCAGGGGAAACAGCAGATTCGCCTGGATG |
| | | ACCAGAAAGAGCGAGGAAACCATCACCCCCTGGAACTTCGAGGAA |
| | | GTGGTGGACAAGGGCGCCAGCGCCCAGAGCTTCATCGAGCGGAT |
| | | GACCAACTTCGATAAGAACCTGCCCAACGAGAAGGTGCTGCCCAA |
| | | GCACAGCCTGCTGTACGAGTACTTCACCGTGTACAACGAGCTGACC |
| | | AAAGTGAAATACGTGACCGAGGGAATGAGAAAGCCCGCCTTCCTG |
| | | AGCGGCGAGCAGAAAAAAGCCATCGTGGACCTGCTGTTCAAGACC |
| | | AACCGGAAAGTGACCGTGAAGCAGCTGAAAGAGGACTACTTCAA |
| | | GAAAATCGAGTGCTTCGACTCCGTGGAAATCTCCGGCGTGGAAGA |
| | | TCGGTTCAACGCCTCCCTGGGCACATACCACGATCTGCTGAAAATT |
| | | ATCAAGGACAAGGACTTCCTGGACAATGAGGAAAACGAGGACATT |
| | | CTGGAAGATATCGTGCTGACCCTGACACTGTTTGAGGACAGAGAG |
| | | ATGATCGAGGAACGGCTGAAAACCTATGCCCACCTGTTCGACGAC |
| | | AAAGTGATGAAGCAGCTGAAGCGGCGGAGATACACCGGCTGGGG |
| | | CAGGCTGAGCCGGAAGCTGATCAACGGCATCCGGGACAAGCAGT |
| | | CCGGCAAGACAATCCTGGATTTCCTGAAGTCCGACGGCTTCGCCAA |
| | | CAGAAACTTCATGCAGCTGATCCACGACGACAGCCTGACCTTTAAA |
| | | GAGGACATCCAGAAAGCCCAGGTGTCCGGCCAGGGCGATAGCCT |
| | | GCACGAGCACATTGCCAATCTGGCCGGCAGCCCCGCCATTAAGAA |
| | | GGGCATCCTGCAGACAGTGAAGGTGGTGGACGAGCTCGTGAAAG |
| | | TGATGGGCCGGCACAAGCCCGAGAACATCGTGATCGAAATGGCCA |
| | | GAGAGAACCAGACCACCCAGAAGGGACAGAAGAACAGCCGCGAG |
| | | AGAATGAAGCGGATCGAAGAGGGCATCAAAGAGCTGGGCAGCCA |
| | | GATCCTGAAAGAACACCCCGTGGAAAACACCCAGCTGCAGAACGA |
| | | GAAGCTGTACCTGTACTACCTGCAGAATGGGCGGGATATGTACGT |
| | | GGACCAGGAACTGGACATCAACCGGCTGTCCGACTACGATGTGGA |
| | | CGCTATCGTGCCTCAGAGCTTTCTGAAGGACGACTCCATCGATAAC |
| | | AAAGTGCTGACTCGGAGCGACAAGAACCGGGGCAAGAGCGACAA |
| | | CGTGCCCTCCGAAGAGGTCGTGAAGAAGATGAAGAACTACTGGC |
| | | GCCAGCTGCTGAATGCCAAGCTGATTACCCAGAGGAAGTTCGACA |
| | | ATCTGACCAAGGCCGAGAGAGGCGGCCTGAGCGAACTGGATAAG |
| | | GCCGGCTTCATCAAGAGACAGCTGGTGGAAACCCGGCAGATCACA |
| | | AAGCACGTGGCACAGATCCTGGACTCCCGGATGAACACTAAGTAC |
| | | GACGAGAACGACAAACTGATCCGGGAAGTGAAAGTGATCACCCTG |
| | | AAGTCCAAGCTGGTGTCCGATTTCCGGAAGGATTTCCAGTTTTACA |
| | | AAGTGCGCGAGATCAACAACTACCACCACGCCCACGACGCCTACCT |
| | | GAACGCCGTCGTGGGAACCGCCCTGATCAAAAAGTACCCTAAGCT |
| | | GGAAAGCGAGTTCGTGTACGGCGACTACAAGGTGTACGACGTGC |
| | | GGAAGATGATCGCCAAGAGCGAGCAGGAAATCGGCAAGGCTACC |
| | | GCCAAGTACTTCTTCTACAGCAACATCATGAACTTTTTCAAGACCG |
| | | AGATTACCCTGGCCAACGGCGAGATCCGGAAGCGGCCTCTGATCG |
| | | AGACAAACGGCGAAACAGGCGAGATCGTGTGGGATAAGGGCCGG |
| | | GACTTTGCCACCGTGCGGAAAGTGCTGTCTATGCCCCAAGTGAATA |
| | | TCGTGAAAAAGACCGAGGTGCAGACAGGCGGCTTCAGCAAAGAG |
| | | TCTATCCTGCCCAAGAGGAACAGCGACAAGCTGATCGCCAGAAAG |
| | | AAGGACTGGGACCCTAAGAAGTACGGCGGCTTCGACAGCCCCACC |
| | | GTGGCCTATTCTGTGCTGGTGGTGGCCAAAGTGGAAAAGGGCAA |
| | | GTCCAAGAAACTGAAGAGTGTGAAAGAGCTGCTGGGGATCACCAT |
| | | CATGGAAAGAAGCAGCTTCGAGAAGAATCCCATCGACTTTCTGGA |
| | | AGCCAAGGGCTACAAAGAAGTGAAAAAGGACCTGATCATCAAGCT |
| | | GCCTAAGTACTCCCTGTTCGAGCTGGAAAACGGCCGGAAGAGAAT |
| | | GCTGGCCTCTGCCGGCGAACTGCAGAAGGGAAACGAACTGGCCCT |
| | | GCCCTCCAAATATGTGAACTTCCTGTACCTGGCCAGCCACTATGAG |
| | | AAGCTGAAGGGCTCCCCCGAGGATAATGAGCAGAAACAGCTGTTT |
| | | GTGGAACAGCACAAACACTACCTGGACGAGATCATCGAGCAGATC |
| | | AGCGAGTTCTCCAAGAGAGTGATCCTGGCCGACGCTAATCTGGAC |
| | | AAGGTGCTGAGCGCCTACAACAAGCACAGAGACAAGCCTATCAGA |
| | | GAGCAGGCCGAGAATATCATCCACCTGTTTACCCTGACCAATCTGG |
| | | GAGCCCCTGCCGCCTTCAAGTACTTTGACACCACCATCGACCGGAA |
| | | GAGGTACACCAGCACCAAAGAGGTGCTGGACGCCACCCTGATCCA |
| | | CCAGAGCATCACCGGCCTGTACGAGACACGGATCGACCTGTCTCA |
| | | GCTGGGAGGCGACCCAAAAAAGAAGAGAAAGGTAAGCGGAAGT |
| | | GAGACCCCAGGTACATCCGAATCAGCAACGCCTGAAAGCACCGGT |
| | | CGGACACTGGTGACCTTCAAGGATGTATTTGTGGACTTCACCAGG |
| | | GAGGAGTGGAAGCTGCTGGACACTGCTCAGCAGATCGTGTACAG |
| | | AAATGTGATGCTGGAGAACTATAAGAACCTGGTTTCCTTGGGTTAT |
| | | CAGCTTACTAAGCCAGATGTGATCCTCCGGTTGGAGAAGGGAGAA |
| | | GAGCCCTGA |
| | Fusion Protein | MGTMPKKKRKVPKKKRKVYNHDQEFDPPKVYPPVPAEKRKPIRVLSL |
| | 15 Amino Acid | FDGIATGLLVLKDLGIQVDRYIASEVCEDSITVGMVRHQGKIMYVGDV |
| | Sequence | RSVTQKHIQEWGPFDLVIGGSPCNDLSIVNPARKGLYEGTGRLFFEFY |
| | NLS-NLS-3A-3L- | RLLHDARPKEGDDRPFFWLFENVVAMGVSDKRDISRFLESNPVMIDA |
| | dCas9-ZIM3-NLS- | KEVSAAHRARYFWGNLPGMNRPLASTVNDKLELQECLEHGRIAKFSK |
| | NLS | VRTITTRSNSIKQGKDQHFPVFMNEKEDILWCTEMERVFGFPVHYTD |
| | | VSNMSRLARQRLLGRSWSVPVIRHLFAPLKEYFACVSSGNSNANSRG |
| | | PSFSSGLVPLSLRGSHMGPMEIYKTVSAWKRQPVRVLSLFRNIDKVLK |
| | | SLGFLESGSGSGGGTLKYVEDVTNVVRRDVEKWGPFDLVYGSTQPLG |
| | | SSCDRCPGWYMFQFHRILQYALPRQESQRPFFWIFMDNLLLTEDDQ |
| | | ETTTRFLQTEAVTLQDVRGRDYQNAMRVWSNIPGLKSKHAPLTPKEE |
| | | EYLQAQVRSRSKLDAPKVDLLVKNCLLPLREYFKYFSQNSLPLGGPSSG |
| | | APPPSGGSPAGSPTSTEEGTSESATPESGPGTSTEPSEGSAPGSPAGSP |
| | | TSTEEGTSTEPSEGSAPGTSTEPSEMDKKYSIGLAIGTNSVGWAVITDE |
| | | YKVPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGNI |
| | | VDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRR |
| | | LENLIAQLPGEKKNGLFGNLIALSLGLTPNFKSNFDLAEDAKLQLSKDTY |
| | | DDDLDNLLAQIGDQYADLFLAAKNLSDAILLSDILRVNTEITKAPLSAS |
| | | MIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGE |
| | | LHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQ |
| | | LKEDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGWGRL |
| | | SRKLINGIRDKQSGKTILDFLKSDGFANRNFMQLIHDDSLTFKEDIQKA |
| | | QVSGQGDSLHEHIANLAGSPAIKKGILQTVKVVDELVKVMGRHKPEN |
| | | IVIEMARENQTTQKGQKNSRERMKRIEEGIKELGSQILKEHPVENTQL |
| | | QNEKLYLYYLQNGRDMYVDQELDINRLSDYDVDAIVPQSFLKDDSID |
| | | NKVLTRSDKNRGKSDNVPSEEVVKKMKNYWRQLLNAKLITQRKFDN |
| | | LTKAERGGLSELDKAGFIKRQLVETRQITKHVAQILDSRMNTKYDEND |
| | | KLIREVKVITLKSKLVSDFRKDFQFYKVREINNYHHAHDAYLNAVVGTA |
| | | LIKKYPKLESEFVYGDYKVYDVRKMIAKSEQEIGKATAKYFFYSNIMNFF |
| | | KTEITLANGEIRKRPLIETNGETGEIVWDKGRDFATVRKVLSMPQVNIV |
| | | KKTEVQTGGFSKESILPKRNSDKLIARKKDWDPKKYGGFDSPTVAYSV |
| | | LVVAKVEKGKSKKLKSVKELLGITIMERSSFEKNPIDFLEAKGYKEVKKD |
| | | LIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVL |
| | | DATLIHQSITGLYETRIDLSQLGGDSGSETPGTSESATPESTGMNNSQ |
| | | GRVTFEDVTVNFTQGEWQRLNPEQRNLYRDVMLENYSNLVSVGQG |
| | | ETTKPDVILRLEQGKEPWLEEEEVLGSGRAEKNGDIGGQIWKPKDVKE |
| | | SLSADYKDDDDKAPKKKRKVPKKKRKV |
| | Fusion Protein | ATGGGTACCATGCCAAAAAAGAAGAGAAAGGTACCGAAGAAAAA |
| | 15 DNA | AAGAAAGGTATACAACCATGACCAGGAATTCGACCCCCCAAAGGT |
| | Sequence | TTACCCACCTGTGCCAGCTGAGAAGAGGAAGCCCATCCGCGTGCT |
| | | GTCTCTCTTTGATGGGATTGCTACAGGGCTCCTGGTGCTGAAGGAC |
| | | CTGGGCATCCAAGTGGACCGCTACATTGCCTCCGAGGTGTGTGAG |
| | | GACTCCATCACGGTGGGCATGGTGCGGCACCAGGGAAAGATCAT |
| | | GTACGTCGGGGACGTCCGCAGCGTCACACAGAAGCATATCCAGGA |
| | | GTGGGGCCCATTCGACCTGGTGATTGGAGGCAGTCCCTGCAATGA |
| | | CCTCTCCATTGTCAACCCTGCCCGCAAGGGACTTTATGAGGGTACT |
| | | GGCCGCCTCTTCTTTGAGTTCTACCGCCTCCTGCATGATGCGCGGC |
| | | CCAAGGAGGGAGATGATCGCCCCTTCTTCTGGCTCTTTGAGAATGT |
| | | GGTGGCCATGGGCGTTAGTGACAAGAGGGACATCTCGCGATTTCT |
| | | TGAGTCTAACCCCGTGATGATTGACGCCAAAGAAGTGTCTGCTGC |
| | | ACACAGGGCCCGTTACTTCTGGGGTAACCTTCCTGGCATGAACAG |
| | | GCCTTTGGCATCCACTGTGAATGATAAGCTGGAGCTGCAAGAGTG |
| | | TCTGGAGCACGGCAGAATAGCCAAGTTCAGCAAAGTGAGGACCAT |
| | | TACCACCAGGTCAAACTCTATAAAGCAGGGCAAAGACCAGCATTTC |
| | | CCCGTCTTCATGAACGAGAAGGAGGACATCCTGTGGTGCACTGAA |
| | | ATGGAAAGGGTGTTTGGCTTCCCCGTCCACTACACAGACGTCTCCA |
| | | ACATGAGCCGCTTGGCGAGGCAGAGACTGCTGGGCCGATCGTGG |
| | | AGCGTGCCGGTCATCCGCCACCTCTTCGCTCCGCTGAAGGAATATT |
| | | TTGCTTGTGTGTCTAGCGGCAATAGTAACGCTAACAGCCGCGGGC |
| | | CGAGCTTCAGCAGCGGCCTGGTGCCGTTAAGCTTGCGCGGCAGCC |
| | | ATATGGGCCCTATGGAGATATACAAGACAGTGTCTGCATGGAAGA |
| | | GACAGCCAGTGCGGGTACTGAGCCTCTTCAGAAACATCGACAAGG |
| | | TACTAAAGAGTTTGGGCTTCTTGGAAAGCGGTTCTGGTTCTGGGG |
| | | GAGGAACGCTGAAGTACGTGGAAGATGTCACAAATGTCGTGAGG |
| | | AGAGACGTGGAGAAATGGGGCCCCTTTGACCTGGTGTACGGCTCG |
| | | ACGCAGCCCCTAGGCAGCTCTTGTGATCGCTGTCCCGGCTGGTACA |
| | | TGTTCCAGTTCCACCGGATCCTGCAGTATGCGCTGCCTCGCCAGGA |
| | | GAGTCAGCGGCCCTTCTTCTGGATATTCATGGACAATCTGCTGCTG |
| | | ACTGAGGATGACCAAGAGACAACTACCCGCTTCCTTCAGACAGAG |
| | | GCTGTGACCCTCCAGGATGTCCGTGGCAGAGACTACCAGAATGCT |
| | | ATGCGGGTGTGGAGCAACATTCCAGGGCTGAAGAGCAAGCATGC |
| | | GCCCCTGACCCCAAAGGAAGAAGAGTATCTGCAAGCCCAAGTCAG |
| | | AAGCAGGAGCAAGCTGGACGCCCCGAAAGTTGACCTCCTGGTGAA |
| | | GAACTGCCTTCTCCCGCTGAGAGAGTACTTCAAGTATTTTTCTCAAA |
| | | ACTCACTTCCTCTTGGAGGGCCGAGCTCTGGCGCACCCCCACCAAG |
| | | TGGAGGGTCTCCTGCCGGGTCCCCAACATCTACTGAAGAAGGCAC |
| | | CAGCGAATCCGCAACGCCCGAGTCAGGCCCTGGTACCTCCACAGA |
| | | ACCATCTGAAGGTAGTGCGCCTGGTTCCCCAGCTGGAAGCCCTACT |
| | | TCCACCGAAGAAGGCACGTCAACCGAACCAAGTGAAGGATCTGCC |
| | | CCTGGGACCAGCACTGAACCATCTGAGATGGACAAGAAGTACAGC |
| | | ATCGGCCTGGCCATCGGCACCAACTCTGTGGGCTGGGCCGTGATC |
| | | ACCGACGAGTACAAGGTGCCCAGCAAGAAATTCAAGGTGCTGGGC |
| | | AACACCGACCGGCACAGCATCAAGAAGAACCTGATCGGCGCCCTG |
| | | CTGTTCGACAGCGGAGAAACAGCCGAGGCCACCCGGCTGAAGAG |
| | | AACCGCCAGAAGAAGATACACCAGACGGAAGAACCGGATCTGCTA |
| | | TCTGCAAGAGATCTTCAGCAACGAGATGGCCAAGGTGGACGACAG |
| | | CTTCTTCCACAGACTGGAAGAGTCCTTCCTGGTGGAAGAGGATAA |
| | | GAAGCACGAGCGGCACCCCATCTTCGGCAACATCGTGGACGAGGT |
| | | GGCCTACCACGAGAAGTACCCCACCATCTACCACCTGAGAAAGAA |
| | | ACTGGTGGACAGCACCGACAAGGCCGACCTGCGGCTGATCTATCT |
| | | GGCCCTGGCCCACATGATCAAGTTCCGGGGCCACTTCCTGATCGA |
| | | GGGCGACCTGAACCCCGACAACAGCGACGTGGACAAGCTGTTCAT |
| | | CCAGCTGGTGCAGACCTACAACCAGCTGTTCGAGGAAAACCCCAT |
| | | CAACGCCAGCGGCGTGGACGCCAAGGCCATCCTGTCTGCCAGACT |
| | | GAGCAAGAGCAGACGGCTGGAAAATCTGATCGCCCAGCTGCCCG |
| | | GCGAGAAGAAGAATGGCCTGTTCGGCAACCTGATTGCCCTGAGCC |
| | | TGGGCCTGACCCCCAACTTCAAGAGCAACTTCGACCTGGCCGAGG |
| | | ATGCCAAACTGCAGCTGAGCAAGGACACCTACGACGACGACCTGG |
| | | ACAACCTGCTGGCCCAGATCGGCGACCAGTACGCCGACCTGTTTCT |
| | | GGCCGCCAAGAACCTGTCCGACGCCATCCTGCTGAGCGACATCCT |
| | | GAGAGTGAACACCGAGATCACCAAGGCCCCCCTGAGCGCCTCTAT |
| | | GATCAAGAGATACGACGAGCACCACCAGGACCTGACCCTGCTGAA |
| | | AGCTCTCGTGCGGCAGCAGCTGCCTGAGAAGTACAAAGAGATTTT |
| | | CTTCGACCAGAGCAAGAACGGCTACGCCGGCTACATCGATGGCGG |
| | | AGCCAGCCAGGAAGAGTTCTACAAGTTCATCAAGCCCATCCTGGA |
| | | AAAGATGGACGGCACCGAGGAACTGCTCGTGAAGCTGAACAGAG |
| | | AGGACCTGCTGCGGAAGCAGCGGACCTTCGACAACGGCAGCATCC |
| | | CCCACCAGATCCACCTGGGAGAGCTGCACGCCATTCTGCGGCGGC |
| | | AGGAAGATTTTTACCCATTCCTGAAGGACAACCGGGAAAAGATCG |
| | | AGAAGATCCTGACCTTCCGCATCCCCTACTACGTGGGCCCTCTGGC |
| | | CAGGGGAAACAGCAGATTCGCCTGGATGACCAGAAAGAGCGAGG |
| | | AAACCATCACCCCCTGGAACTTCGAGGAAGTGGTGGACAAGGGCG |
| | | CCAGCGCCCAGAGCTTCATCGAGCGGATGACCAACTTCGATAAGA |
| | | ACCTGCCCAACGAGAAGGTGCTGCCCAAGCACAGCCTGCTGTACG |
| | | AGTACTTCACCGTGTACAACGAGCTGACCAAAGTGAAATACGTGA |
| | | CCGAGGGAATGAGAAAGCCCGCCTTCCTGAGCGGCGAGCAGAAA |
| | | AAAGCCATCGTGGACCTGCTGTTCAAGACCAACCGGAAAGTGACC |
| | | GTGAAGCAGCTGAAAGAGGACTACTTCAAGAAAATCGAGTGCTTC |
| | | GACTCCGTGGAAATCTCCGGCGTGGAAGATCGGTTCAACGCCTCC |
| | | CTGGGCACATACCACGATCTGCTGAAAATTATCAAGGACAAGGAC |
| | | TTCCTGGACAATGAGGAAAACGAGGACATTCTGGAAGATATCGTG |
| | | CTGACCCTGACACTGTTTGAGGACAGAGAGATGATCGAGGAACGG |
| | | CTGAAAACCTATGCCCACCTGTTCGACGACAAAGTGATGAAGCAG |
| | | CTGAAGCGGCGGAGATACACCGGCTGGGGCAGGCTGAGCCGGAA |
| | | GCTGATCAACGGCATCCGGGACAAGCAGTCCGGCAAGACAATCCT |
| | | GGATTTCCTGAAGTCCGACGGCTTCGCCAACAGAAACTTCATGCAG |
| | | CTGATCCACGACGACAGCCTGACCTTTAAAGAGGACATCCAGAAA |
| | | GCCCAGGTGTCCGGCCAGGGCGATAGCCTGCACGAGCACATTGCC |
| | | AATCTGGCCGGCAGCCCCGCCATTAAGAAGGGCATCCTGCAGACA |
| | | GTGAAGGTGGTGGACGAGCTCGTGAAAGTGATGGGCCGGCACAA |
| | | GCCCGAGAACATCGTGATCGAAATGGCCAGAGAGAACCAGACCAC |
| | | CCAGAAGGGACAGAAGAACAGCCGCGAGAGAATGAAGCGGATCG |
| | | AAGAGGGCATCAAAGAGCTGGGCAGCCAGATCCTGAAAGAACAC |
| | | CCCGTGGAAAACACCCAGCTGCAGAACGAGAAGCTGTACCTGTAC |
| | | TACCTGCAGAATGGGCGGGATATGTACGTGGACCAGGAACTGGA |
| | | CATCAACCGGCTGTCCGACTACGATGTGGACGCTATCGTGCCTCAG |
| | | AGCTTTCTGAAGGACGACTCCATCGATAACAAAGTGCTGACTCGG |
| | | AGCGACAAGAACCGGGGCAAGAGCGACAACGTGCCCTCCGAAGA |
| | | GGTCGTGAAGAAGATGAAGAACTACTGGCGCCAGCTGCTGAATGC |
| | | CAAGCTGATTACCCAGAGGAAGTTCGACAATCTGACCAAGGCCGA |
| | | GAGAGGCGGCCTGAGCGAACTGGATAAGGCCGGCTTCATCAAGA |
| | | GACAGCTGGTGGAAACCCGGCAGATCACAAAGCACGTGGCACAG |
| | | ATCCTGGACTCCCGGATGAACACTAAGTACGACGAGAACGACAAA |
| | | CTGATCCGGGAAGTGAAAGTGATCACCCTGAAGTCCAAGCTGGTG |
| | | TCCGATTTCCGGAAGGATTTCCAGTTTTACAAAGTGCGCGAGATCA |
| | | ACAACTACCACCACGCCCACGACGCCTACCTGAACGCCGTCGTGG |
| | | GAACCGCCCTGATCAAAAAGTACCCTAAGCTGGAAAGCGAGTTCG |
| | | TGTACGGCGACTACAAGGTGTACGACGTGCGGAAGATGATCGCCA |
| | | AGAGCGAGCAGGAAATCGGCAAGGCTACCGCCAAGTACTTCTTCT |
| | | ACAGCAACATCATGAACTTTTTCAAGACCGAGATTACCCTGGCCAA |
| | | CGGCGAGATCCGGAAGCGGCCTCTGATCGAGACAAACGGCGAAA |
| | | CAGGCGAGATCGTGTGGGATAAGGGCCGGGACTTTGCCACCGTG |
| | | CGGAAAGTGCTGTCTATGCCCCAAGTGAATATCGTGAAAAAGACC |
| | | GAGGTGCAGACAGGCGGCTTCAGCAAAGAGTCTATCCTGCCCAAG |
| | | AGGAACAGCGACAAGCTGATCGCCAGAAAGAAGGACTGGGACCC |
| | | TAAGAAGTACGGCGGCTTCGACAGCCCCACCGTGGCCTATTCTGT |
| | | GCTGGTGGTGGCCAAAGTGGAAAAGGGCAAGTCCAAGAAACTGA |
| | | AGAGTGTGAAAGAGCTGCTGGGGATCACCATCATGGAAAGAAGC |
| | | AGCTTCGAGAAGAATCCCATCGACTTTCTGGAAGCCAAGGGCTAC |
| | | AAAGAAGTGAAAAAGGACCTGATCATCAAGCTGCCTAAGTACTCC |
| | | CTGTTCGAGCTGGAAAACGGCCGGAAGAGAATGCTGGCCTCTGCC |
| | | GGCGAACTGCAGAAGGGAAACGAACTGGCCCTGCCCTCCAAATAT |
| | | GTGAACTTCCTGTACCTGGCCAGCCACTATGAGAAGCTGAAGGGC |
| | | TCCCCCGAGGATAATGAGCAGAAACAGCTGTTTGTGGAACAGCAC |
| | | AAACACTACCTGGACGAGATCATCGAGCAGATCAGCGAGTTCTCC |
| | | AAGAGAGTGATCCTGGCCGACGCTAATCTGGACAAGGTGCTGAGC |
| | | GCCTACAACAAGCACAGAGACAAGCCTATCAGAGAGCAGGCCGA |
| | | GAATATCATCCACCTGTTTACCCTGACCAATCTGGGAGCCCCTGCC |
| | | GCCTTCAAGTACTTTGACACCACCATCGACCGGAAGAGGTACACCA |
| | | GCACCAAAGAGGTGCTGGACGCCACCCTGATCCACCAGAGCATCA |
| | | CCGGCCTGTACGAGACACGGATCGACCTGTCTCAGCTGGGAGGCG |
| | | ACAGCGGAAGTGAGACCCCAGGTACATCCGAATCAGCAACGCCTG |
| | | AAAGCACCGGTATGAACAATTCACAGGGGAGAGTGACATTCGAAG |
| | | ACGTGACCGTGAACTTCACCCAGGGAGAATGGCAGCGCTTGAACC |
| | | CAGAACAAAGGAACCTCTATCGGGACGTGATGCTGGAAAACTACT |
| | | CAAATTTGGTGAGCGTTGGGCAGGGTGAGACCACTAAGCCTGACG |
| | | TGATCCTGAGATTGGAACAGGGCAAGGAGCCTTGGCTCGAGGAA |
| | | GAGGAAGTCCTGGGCTCAGGGAGGGCCGAGAAAAACGGTGATAT |
| | | AGGAGGCCAGATATGGAAGCCTAAGGACGTCAAGGAGAGCCTGA |
| | | GCGCTGATTACAAAGATGATGACGATAAAGCCCCAAAAAAGAAGA |
| | | GAAAGGTACCGAAGAAAAAAAGAAAGGTCTGA |
| | Fusion Protein | MYPYDVPDYASPKKKRKVGGGASMGRVTFEDVTVNFTQGEWQRLN |
| | Configuration 16 | PEQRNLYRDVMLENYSNLVSVGQGETTKPDVILRLEQGKEPWLEEEE |
| | | VLGSGRAEKNGDIGGQIWKPKDVKESLGGGGSGGGGSGGGGSGGG |
| | | GSLEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSGGGMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSL |
| | Fusion Protein | MYPYDVPDYASPKKKRKVGGGASMGRVTFEDVTVNFTQGEWQRLN |
| | Configuration 17 | PEQRNLYRDVMLENYSNLVSVGQGETTKPDVILRLEQGKEPWLEEEE |
| | | VLGSGRAEKNGDIGGQIWKPKDVKESLGGGGSGGGGGGGGSGGG |
| | | GSLEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSGGGMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSL |
| | Fusion Protein | MYPYDVPDYASPKKKRKVGGGASMGRVTFEDVTVNFTQGEWQRLN |
| | Configuration 18 | PEQRNLYRDVMLENYSNLVSVGQGETTKPDVILRLEQGKEPWLEEEE |
| | | VLGSGRAEKNGDIGGQIWKPKDVKESLGGGGSGGGGSGGGGSGGG |
| | | GSLEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSGGGMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSL |
| | Fusion Protein | MYPYDVPDYASPKKKRKVGGGASMGRVTFEDVTVNFTQGEWQRLN |
| | Configuration 19 | PEQRNLYRDVMLENYSNLVSVGQGETTKPDVILRLEQGKEPWLEEEE |
| | | VLGSGRAEKNGDIGGQIWKPKDVKESLGGGGSGGGGSGGGGSGGG |
| | | GSLEDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIAL |
| | | SLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKIEK |
| | | ILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPA |
| | | FLSGEQKKAIVDLLFKTNRKVTVKQLKEDYFKKIECFDSVEISGVEDRFN |
| | | ASLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKK |
| | | GILQTVKVVDELVKVMGRHKPENIVIEMARENQTTQKGQKNSRERM |
| | | KRIEEGIKELGSQILKEHPVENTQLQNEKLYLYYLQNGRDMYVDQELDI |
| | | NRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETR |
| | | QITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGITIM |
| | | ERSSFEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYLDE |
| | | IIEQISEFSKRVILADANLDKVLSAYNKHRDKPIREQAENIIHLFTLTNLG |
| | | APAAFKYFDTTIDRKRYTSTKEVLDATLIHQSITGLYETRIDLSQLGGDS |
| | | PKKKRKVGVDGSGGGMAAIPALDPEAEPSMDVILVGSSELSSSVSPGT |
| | | GRDLIAYEVKANQRNIEDICICCGSLQVHTQHPLFEGGICAPCKDKFLD |
| | | ALFLYDDDGYQSYCSICCSGETLLICGNPDCTRCYCFECVDSLVGPGTS |
| | | GKVHAMSNWVCYLCLPSSRSGLLQRRRKWRSQLKAFYDRESENPLE |
| | | MFETVPVWRRQPVRVLSLFEDIKKELTSLGFLESGSDPGQLKHVVDVT |
| | | DTVRKDVEEWGPFDLVYGATPPLGHTCDRPPSWYLFQFHRLLQYAR |
| | | PKPGSPRPFFWMFVDNLVLNKEDLDVASRFLEMEPVTIPDVHGGSLQ |
| | | NAVRVWSNIPAIRSRHWALVSEEELSLLAQNKQSSKLAAKWPTKLVK |
| | | NCFLPLREYFKYFSTELTSSL |
| 667 | Fusion protein | MPKKKRKVPKKKRKVYNHDQEFDPPKVYPPVPAEKRK |
| | configuration 11a | PIRVLSLFDGIATGLLVLKDLGIQVDRYIASEVCEDSITVG |
| | | MVRHQGKIMYVGDVRSVTQKHIQEWGPFDLVIGGSPCN |
| | | DLSIVNPARKGLYEGTGRLFFEFYRLLHDARPKEGDDRP |
| | | FFWLFENVVAMGVSDKRDISRFLESNPVMIDAKEVSAA |
| | | HRARYFWGNLPGMNRPLASTVNDKLELQECLEHGRIAK |
| | | FSKVRTITTRSNSIKQGKDQHFPVFMNEKEDILWCTEME |
| | | RVFGFPVHYTDVSNMSRLARQRLLGRSWSVPVIRHLFAP |
| | | LKEYFACVSSGNSNANSRGPSFSSGLVPLSLRGSHMAAIP |
| | | ALDPEAEPSMDVILVGSSELSSSVSPGTGRDLIAYEVKAN |
| | | QRNIEDICICCGSLQVHTQHPLFEGGICAPCKDKFLDALF |
| | | LYDDDGYQSYCSICCSGETLLICGNPDCTRCYCFECVDS |
| | | LVGPGTSGKVHAMSNWVCYLCLPSSRSGLLQRRRKWRS |
| | | QLKAFYDRESENPLEMFETVPVWRRQPVRVLSLFEDIKK |
| | | ELTSLGFLESGSDPGQLKHVVDVTDTVRKDVEEWGPFD |
| | | LVYGATPPLGHTCDRPPSWYLFQFHRLLQYARPKPGSPR |
| | | PFFWMFVDNLVLNKEDLDVASRFLEMEPVTIPDVHGGS |
| | | LQNAVRVWSNIPAIRSRHWALVSEEELSLLAQNKQSSKL |
| | | AAKWPTKLVKNCFLPLREYFKYFSTELTSSLGGPSSGAP |
| | | PPSGGSPAGSPTSTEEGTSESATPESGPGTSTEPSEGSAPG |
| | | SPAGSPTSTEEGTSTEPSEGSAPGTSTEPSELEDKKYSIGL |
| | | AIGTNSVGWAVITDEYKVPSKKFKVLGNTDRHSIKKNLI |
| | | GALLFDSGETAEATRLKRTARRRYTRRKNRICYLQEIFSN |
| | | EMAKVDDSFFHRLEESFLVEEDKKHERHPIFGNIVDEVA |
| | | YHEKYPTIYHLRKKLVDSTDKADLRLIYLALAHMIKFRG |
| | | HFLIEGDLNPDNSDVDKLFIQLVQTYNQLFEENPINASGV |
| | | DAKAILSARLSKSRRLENLIAQLPGEKKNGLFGNLIALSL |
| | | GLTPNFKSNFDLAEDAKLQLSKDTYDDDLDNLLAQIGD |
| | | QYADLFLAAKNLSDAILLSDILRVNTEITKAPLSASMIKR |
| | | YDEHHQDLTLLKALVRQQLPEKYKEIFFDQSKNGYAGYI |
| | | DGGASQEEFYKFIKPILEKMDGTEELLVKLNREDLLRKQ |
| | | RTFDNGSIPHQIHLGELHAILRRQEDFYPFLKDNREKIEKI |
| | | LTFRIPYYVGPLARGNSRFAWMTRKSEETITPWNFEEVV |
| | | DKGASAQSFIERMTNFDKNLPNEKVLPKHSLLYEYFTVY |
| | | NELTKVKYVTEGMRKPAFLSGEQKKAIVDLLFKTNRKV |
| | | TVKQLKEDYFKKIECFDSVEISGVEDRFNASLGTYHDLL |
| | | KIIKDKDFLDNEENEDILEDIVLTLTLFEDREMIEERLKTY |
| | | AHLFDDKVMKQLKRRRYTGWGRLSRKLINGIRDKQSGK |
| | | TILDFLKSDGFANRNFMQLIHDDSLTFKEDIQKAQVSGQ |
| | | GDSLHEHIANLAGSPAIKKGILQTVKVVDELVKVMGRH |
| | | KPENIVIEMARENQTTQKGQKNSRERMKRIEEGIKELGS |
| | | QILKEHPVENTQLQNEKLYLYYLQNGRDMYVDQELDIN |
| | | RLSDYDVDAIVPQSFLKDDSIDNKVLTRSDKNRGKSDNV |
| | | PSEEVVKKMKNYWRQLLNAKLITQRKFDNLTKAERGGL |
| | | SELDKAGFIKRQLVETRQITKHVAQILDSRMNTKYDEND |
| | | KLIREVKVITLKSKLVSDFRKDFQFYKVREINNYHHAHD |
| | | AYLNAVVGTALIKKYPKLESEFVYGDYKVYDVRKMIAK |
| | | SEQEIGKATAKYFFYSNIMNFFKTEITLANGEIRKRPLIET |
| | | NGETGEIVWDKGRDFATVRKVLSMPQVNIVKKTEVQTG |
| | | GFSKESILPKRNSDKLIARKKDWDPKKYGGFDSPTVAYS |
| | | VLVVAKVEKGKSKKLKSVKELLGITIMERSSFEKNPIDFL |
| | | EAKGYKEVKKDLIIKLPKYSLFELENGRKRMLASAGELQ |
| | | KGNELALPSKYVNFLYLASHYEKLKGSPEDNEQKQLFV |
| | | EQHKHYLDEIIEQISEFSKRVILADANLDKVLSAYNKHRD |
| | | KPIREQAENIIHLFTLTNLGAPAAFKYFDTTIDRKRYTSTK |
| | | EVLDATLIHQSITGLYETRIDLSQLGGDSPKKKRKVGVD |
| | | GSSGSETPGTSESATPESTGMNNSQGRVTFEDVTVNFTQ |
| | | GEWQRLNPEQRNLYRDVMLENYSNLVSVGQGETTKPD |
| | | VILRLEQGKEPWLEEEEVLGSGRAEKNGDIGGQIWKPKD |
| | | VKESLSADYKDDDDKAPKKKRKVPKKKRKV |
| 668 | Polynucleotide | ATGCCAAAAAAGAAGAGAAAGGTACCGAAGAAAAAA |
| | Encoding Fusion | AGAAAGGTATACAATCACGATCAGGAGTTCGACCCCC |
| | Protein | CTAAGGTGTACCCACCAGTGCCTGCAGAGAAGAGGAA |
| | Configuration 11a | GCCAATCCGGGTGCTGAGCCTGTTTGATGGCATCGCC |
| | | ACCGGCCTGCTGGTGCTGAAGGATCTGGGCATCCAGG |
| | | TGGACCGGTACATCGCCTCCGAGGTGTGCGAGGATTC |
| | | TATCACCGTGGGCATGGTGCGCCACCAGGGCAAGATC |
| | | ATGTATGTGGGCGACGTGCGGTCCGTGACACAGAAGC |
| | | ACATCCAGGAGTGGGGCCCATTCGATCTGGTGATCGG |
| | | CGGCAGCCCCTGTAATGACCTGTCCATCGTGAACCCT |
| | | GCAAGGAAGGGACTGTACGAGGGAACCGGCCGGCTG |
| | | TTCTTTGAGTTTTATAGACTGCTGCACGACGCCAGGCC |
| | | TAAGGAGGGCGACGATAGACCATTCTTTTGGCTGTTC |
| | | GAGAATGTGGTGGCTATGGGCGTGAGCGATAAGAGG |
| | | GACATCTCCAGGTTTCTGGAGTCTAACCCCGTGATGAT |
| | | CGATGCAAAGGAGGTGTCCGCCGCACACAGAGCCAG |
| | | GTATTTCTGGGGCAATCTGCCAGGAATGAACAGGCCA |
| | | CTGGCAAGCACCGTGAATGACAAGCTGGAGCTGCAGG |
| | | AGTGCCTGGAGCACGGAAGGATCGCCAAGTTTTCCAA |
| | | GGTGCGCACAATCACCACACGGAGCAATTCCATCAAG |
| | | CAGGGCAAGGATCAGCACTTCCCCGTGTTCATGAACG |
| | | AGAAGGAGGACATCCTGTGGTGTACCGAGATGGAGA |
| | | GAGTGTTCGGCTTTCCAGTGCACTACACAGACGTGTCT |
| | | AACATGAGCAGGCTGGCAAGGCAGCGGCTGCTGGGC |
| | | AGATCTTGGAGCGTGCCCGTGATCAGGCACCTGTTCG |
| | | CCCCTCTGAAGGAGTATTTTGCCTGCGTGAGCAGCGG |
| | | CAACTCCAATGCCAACAGCCGGGGCCCCTCTTTCAGC |
| | | TCCGGATTGGTGCCTCTGAGCCTGAGGGGCTCCCACA |
| | | TGGCAGCAATCCCCGCCCTGGACCCCGAGGCCGAGCC |
| | | TAGCATGGACGTGATCCTGGTGGGCTCTAGCGAGCTG |
| | | TCCTCTAGCGTGTCTCCAGGAACCGGAAGGGATCTGA |
| | | TCGCATACGAGGTGAAGGCCAATCAGCGGAACATCGA |
| | | GGACATCTGTATCTGCTGTGGCAGCCTGCAGGTGCAC |
| | | ACACAGCACCCACTGTTCGAGGGAGGAATCTGCGCAC |
| | | CCTGTAAGGATAAGTTCCTGGACGCCCTGTTTCTGTAC |
| | | GACGATGACGGCTACCAGTCCTATTGCTCTATCTGCTG |
| | | TTCCGGCGAGACCCTGCTGATCTGCGGCAATCCAGAT |
| | | TGTACAAGGTGCTATTGTTTTGAGTGCGTGGACTCTCT |
| | | GGTGGGACCAGGCACCAGCGGAAAGGTGCACGCCAT |
| | | GTCCAACTGGGTGTGCTACCTGTGCCTGCCATCCTCTC |
| | | GCAGCGGACTGCTGCAGCGGAGAAGGAAGTGGAGAT |
| | | CCCAGCTGAAGGCCTTCTATGATAGGGAGTCTGAGAA |
| | | CCCCCTGGAGATGTTTGAGACCGTGCCAGTGTGGCGC |
| | | CGGCAGCCCGTGAGGGTGCTGAGCCTGTTCGAGGATA |
| | | TCAAGAAGGAGCTGACATCCCTGGGCTTTCTGGAGTC |
| | | CGGCTCTGACCCCGGACAGCTGAAGCACGTGGTGGAT |
| | | GTGACCGACACAGTGCGGAAGGATGTGGAGGAGTGG |
| | | GGCCCTTTCGACCTGGTGTACGGAGCAACCCCTCCACT |
| | | GGGACACACATGCGACAGACCCCCTTCTTGGTACCTG |
| | | TTCCAGTTTCACCGCCTGCTGCAGTATGCAAGGCCAA |
| | | AGCCAGGCAGCCCTAGACCATTCTTTTGGATGTTCGTG |
| | | GATAATCTGGTGCTGAACAAGGAGGATCTGGACGTGG |
| | | CCAGCAGGTTTCTGGAGATGGAGCCAGTGACCATCCC |
| | | AGACGTGCACGGCGGCTCCCTGCAGAATGCCGTGCGC |
| | | GTGTGGTCTAACATCCCTGCCATCAGAAGCAGGCACT |
| | | GGGCACTGGTGAGCGAGGAGGAGCTGTCCCTGCTGGC |
| | | CCAGAATAAGCAGAGCAGCAAGCTGGCCGCCAAGTG |
| | | GCCTACAAAGCTGGTGAAGAACTGCTTCCTGCCACTG |
| | | CGGGAGTACTTCAAGTATTTTTCCACCGAGCTGACATC |
| | | TAGCCTGGGAGGACCCTCCTCTGGCGCCCCACCACCT |
| | | AGCGGCGGCTCCCCTGCCGGCTCTCCAACCAGCACAG |
| | | AGGAGGGCACCAGCGAGTCCGCCACACCAGAGTCTGG |
| | | ACCTGGCACCAGCACAGAGCCATCCGAGGGCTCTGCC |
| | | CCAGGCTCTCCTGCAGGCAGCCCTACCTCCACCGAAG |
| | | AGGGCACCAGCACAGAGCCTTCTGAGGGCAGCGCCCC |
| | | AGGCACCTCTACAGAGCCAAGCGAGCTCGAGGACAA |
| | | GAAGTACAGCATCGGCCTGGCCATCGGCACCAACTCT |
| | | GTGGGCTGGGCCGTGATCACCGACGAGTACAAGGTGC |
| | | CCAGCAAGAAATTCAAGGTGCTGGGCAACACCGACCG |
| | | GCACAGCATCAAGAAGAACCTGATCGGAGCCCTGCTG |
| | | TTCGACAGCGGCGAAACAGCCGAGGCCACCCGGCTGA |
| | | AGAGAACCGCCAGAAGAAGATACACCAGACGGAAGA |
| | | ACCGGATCTGCTATCTGCAAGAGATCTTCAGCAACGA |
| | | GATGGCCAAGGTGGACGACAGCTTCTTCCACAGACTG |
| | | GAAGAGTCCTTCCTGGTGGAAGAGGATAAGAAGCACG |
| | | AGCGGCACCCCATCTTCGGCAACATCGTGGACGAGGT |
| | | GGCCTACCACGAGAAGTACCCCACCATCTACCACCTG |
| | | AGAAAGAAACTGGTGGACAGCACCGACAAGGCCGAC |
| | | CTGCGGCTGATCTATCTGGCCCTGGCCCACATGATCAA |
| | | GTTCCGGGGCCACTTCCTGATCGAGGGCGACCTGAAC |
| | | CCCGACAACAGCGACGTGGACAAGCTGTTCATCCAGC |
| | | TGGTGCAGACCTACAACCAGCTGTTCGAGGAAAACCC |
| | | CATCAACGCCAGCGGCGTGGACGCCAAGGCCATCCTG |
| | | TCTGCCAGACTGAGCAAGAGCAGACGGCTGGAAAATC |
| | | TGATCGCCCAGCTGCCCGGCGAGAAGAAGAATGGCCT |
| | | GTTCGGCAACCTGATTGCCCTGAGCCTGGGCCTGACC |
| | | CCCAACTTCAAGAGCAACTTCGACCTGGCCGAGGATG |
| | | CCAAACTGCAGCTGAGCAAGGACACCTACGACGACGA |
| | | CCTGGACAACCTGCTGGCCCAGATCGGCGACCAGTAC |
| | | GCCGACCTGTTTCTGGCCGCCAAGAACCTGTCCGACG |
| | | CCATCCTGCTGAGCGACATCCTGAGAGTGAACACCGA |
| | | GATCACCAAGGCCCCCCTGAGCGCCTCTATGATCAAG |
| | | AGATACGACGAGCACCACCAGGACCTGACCCTGCTGA |
| | | AAGCTCTCGTGCGGCAGCAGCTGCCTGAGAAGTACAA |
| | | AGAGATTTTCTTCGACCAGAGCAAGAACGGCTACGCC |
| | | GGCTACATTGACGGCGGAGCCAGCCAGGAAGAGTTCT |
| | | ACAAGTTCATCAAGCCCATCCTGGAAAAGATGGACGG |
| | | CACCGAGGAACTGCTCGTGAAGCTGAACAGAGAGGA |
| | | CCTGCTGCGGAAGCAGCGGACCTTCGACAACGGCAGC |
| | | ATCCCCCACCAGATCCACCTGGGAGAGCTGCACGCCA |
| | | TTCTGCGGCGGCAGGAAGATTTTTACCCATTCCTGAAG |
| | | GACAACCGGGAAAAGATCGAGAAGATCCTGACCTTCC |
| | | GCATCCCCTACTACGTGGGCCCTCTGGCCAGGGGAAA |
| | | CAGCAGATTCGCCTGGATGACCAGAAAGAGCGAGGA |
| | | AACCATCACCCCCTGGAACTTCGAGGAAGTGGTGGAC |
| | | AAGGGCGCTTCCGCCCAGAGCTTCATCGAGCGGATGA |
| | | CCAACTTCGATAAGAACCTGCCCAACGAGAAGGTGCT |
| | | GCCCAAGCACAGCCTGCTGTACGAGTACTTCACCGTG |
| | | TATAACGAGCTGACCAAAGTGAAATACGTGACCGAGG |
| | | GAATGAGAAAGCCCGCCTTCCTGAGCGGCGAGCAGAA |
| | | AAAGGCCATCGTGGACCTGCTGTTCAAGACCAACCGG |
| | | AAAGTGACCGTGAAGCAGCTGAAAGAGGACTACTTCA |
| | | AGAAAATCGAGTGCTTCGACTCCGTGGAAATCTCCGG |
| | | CGTGGAAGATCGGTTCAACGCCTCCCTGGGCACATAC |
| | | CACGATCTGCTGAAAATTATCAAGGACAAGGACTTCC |
| | | TGGACAATGAGGAAAACGAGGACATTCTGGAAGATAT |
| | | CGTGCTGACCCTGACACTGTTTGAGGACAGAGAGATG |
| | | ATCGAGGAACGGCTGAAAACCTATGCCCACCTGTTCG |
| | | ACGACAAAGTGATGAAGCAGCTGAAGCGGCGGAGAT |
| | | ACACCGGCTGGGGCAGGCTGAGCCGGAAGCTGATCAA |
| | | CGGCATCCGGGACAAGCAGTCCGGCAAGACAATCCTG |
| | | GATTTCCTGAAGTCCGACGGCTTCGCCAACAGAAACT |
| | | TCATGCAGCTGATCCACGACGACAGCCTGACCTTTAA |
| | | AGAGGACATCCAGAAAGCCCAGGTGTCCGGCCAGGG |
| | | CGATAGCCTGCACGAGCACATTGCCAATCTGGCCGGC |
| | | AGCCCCGCCATTAAGAAGGGCATCCTGCAGACAGTGA |
| | | AGGTGGTGGACGAGCTCGTGAAAGTGATGGGCCGGCA |
| | | CAAGCCCGAGAACATCGTGATCGAAATGGCCAGAGA |
| | | GAACCAGACCACCCAGAAGGGACAGAAGAACAGCCG |
| | | CGAGAGAATGAAGCGGATCGAAGAGGGCATCAAAGA |
| | | GCTGGGCAGCCAGATCCTGAAAGAACACCCCGTGGAA |
| | | AACACCCAGCTGCAGAACGAGAAGCTGTACCTGTACT |
| | | ACCTGCAGAATGGGCGGGATATGTACGTGGACCAGGA |
| | | ACTGGACATCAACCGGCTGTCCGACTACGATGTGGAC |
| | | GCCATCGTGCCTCAGAGCTTTCTGAAGGACGACTCCA |
| | | TCGACAACAAGGTGCTGACCAGAAGCGACAAGAACC |
| | | GGGGCAAGAGCGACAACGTGCCCTCCGAAGAGGTCGT |
| | | GAAGAAGATGAAGAACTACTGGCGGCAGCTGCTGAA |
| | | CGCCAAGCTGATTACCCAGAGAAAGTTCGACAATCTG |
| | | ACCAAGGCCGAGAGAGGCGGCCTGAGCGAACTGGAT |
| | | AAGGCCGGCTTCATCAAGAGACAGCTGGTGGAAACCC |
| | | GGCAGATCACAAAGCACGTGGCACAGATCCTGGACTC |
| | | CCGGATGAACACTAAGTACGACGAGAATGACAAGCTG |
| | | ATCCGGGAAGTGAAAGTGATCACCCTGAAGTCCAAGC |
| | | TGGTGTCCGATTTCCGGAAGGATTTCCAGTTTTACAAA |
| | | GTGCGCGAGATCAACAACTACCACCACGCCCACGACG |
| | | CCTACCTGAACGCCGTCGTGGGAACCGCCCTGATCAA |
| | | AAAGTACCCTAAGCTGGAAAGCGAGTTCGTGTACGGC |
| | | GACTACAAGGTGTACGACGTGCGGAAGATGATCGCCA |
| | | AGAGCGAGCAGGAAATCGGCAAGGCTACCGCCAAGT |
| | | ACTTCTTCTACAGCAACATCATGAACTTTTTCAAGACC |
| | | GAGATTACCCTGGCCAACGGCGAGATCCGGAAGCGGC |
| | | CTCTGATCGAGACAAACGGCGAAACCGGGGAGATCGT |
| | | GTGGGATAAGGGCCGGGATTTTGCCACCGTGCGGAAA |
| | | GTGCTGAGCATGCCCCAAGTGAATATCGTGAAAAAGA |
| | | CCGAGGTGCAGACAGGCGGCTTCAGCAAAGAGTCTAT |
| | | CCTGCCCAAGAGGAACAGCGATAAGCTGATCGCCAGA |
| | | AAGAAGGACTGGGACCCTAAGAAGTACGGCGGCTTCG |
| | | ACAGCCCCACCGTGGCCTATTCTGTGCTGGTGGTGGCC |
| | | AAAGTGGAAAAGGGCAAGTCCAAGAAACTGAAGAGT |
| | | GTGAAAGAGCTGCTGGGGATCACCATCATGGAAAGAA |
| | | GCAGCTTCGAGAAGAATCCCATCGACTTTCTGGAAGC |
| | | CAAGGGCTACAAAGAAGTGAAAAAGGACCTGATCAT |
| | | CAAGCTGCCTAAGTACTCCCTGTTCGAGCTGGAAAAC |
| | | GGCCGGAAGAGAATGCTGGCCTCTGCCGGCGAACTGC |
| | | AGAAGGGAAACGAACTGGCCCTGCCCTCCAAATATGT |
| | | GAACTTCCTGTACCTGGCCAGCCACTATGAGAAGCTG |
| | | AAGGGCTCCCCCGAGGATAATGAGCAGAAACAGCTGT |
| | | TTGTGGAACAGCACAAGCACTACCTGGACGAGATCAT |
| | | CGAGCAGATCAGCGAGTTCTCCAAGAGAGTGATCCTG |
| | | GCCGACGCTAATCTGGACAAAGTGCTGTCCGCCTACA |
| | | ACAAGCACCGGGATAAGCCCATCAGAGAGCAGGCCG |
| | | AGAATATCATCCACCTGTTTACCCTGACCAATCTGGGA |
| | | GCCCCTGCCGCCTTCAAGTACTTTGACACCACCATCGA |
| | | CCGGAAGAGGTACACCAGCACCAAAGAGGTGCTGGA |
| | | CGCCACCCTGATCCACCAGAGCATCACCGGCCTGTAC |
| | | GAGACACGGATCGACCTGTCTCAGCTGGGAGGCGACA |
| | | GCCCCAAGAAGAAGAGAAAGGTGGGAGTCGACGGAT |
| | | CCAGCGGCTCCGAGACCCCAGGCACATCTGAGAGCGC |
| | | CACCCCTGAGTCCACCGGTATGAACAATTCACAGGGG |
| | | AGAGTGACATTCGAAGACGTGACCGTGAACTTCACCC |
| | | AGGGAGAATGGCAGCGCTTGAACCCAGAACAAAGGA |
| | | ACCTCTATCGGGACGTGATGCTGGAAAACTACTCAAA |
| | | TTTGGTGAGCGTTGGGCAGGGTGAGACCACTAAGCCT |
| | | GACGTGATCCTGAGATTGGAACAGGGCAAGGAGCCTT |
| | | GGCTCGAGGAAGAGGAAGTCCTGGGCTCAGGGAGGG |
| | | CCGAGAAAAACGGTGATATAGGAGGCCAGATATGGA |
| | | AGCCTAAGGACGTCAAGGAGAGCCTGAGCGCTGATTA |
| | | CAAAGATGATGACGATAAAGCCCCCAAGAAGAAAAG |
| | | GAAGGTCCCAAAGAAAAAAAGAAAGGTG |

1. A system for repressing transcription of a human CIITA gene in a human cell, optionally a human T lymphocyte or a human NK cell, comprising
a) one or more fusion proteins that collectively comprise
a DNA methyltransferase (DNMT) domain and/or a domain that recruits a DNMT, optionally wherein the DNMT domain and/or the recruiter domain comprise a DNMT3A domain and/or
a DNMT3L domain, and optionally wherein the recruited DNMT is DNMT3A, and
a transcriptional repressor domain,
each domain being linked to a DNA-binding domain that binds to a target region in the human CIITA gene, or
b) one or more nucleic acid molecules encoding the one or more fusion proteins.

2. The system of claim 1, wherein the system comprises
a) a single fusion protein comprising the DNMT3A domain, the DNMT3L domain, the transcriptional repressor domain, and the DNA-binding domain, or
b) a nucleic acid molecule encoding the single fusion protein.

3. The system of claim 1 or 2, wherein the DNA-binding domain comprises a dead CRISPR Cas (dCas) domain, a ZFP domain, or a TALE domain.

4. The system of claim 3, wherein the DNA-binding domain comprises a dCas9 domain and the system further comprises (i) one or more guide RNAs comprising any one of SEQ ID NOs: 1034-1312, or (ii) nucleic acid molecules coding for the one or more guide RNAs.

5. The system of any one of claims 3-4, wherein the dCas domain comprises a dCas9 sequence, optionally a sequence with at least 90% identity to SEQ ID NO: 12 or 13.

6. The system of any one of claims 1-5, wherein the DNA-binding domain binds to a target sequence in SEQ ID NO: 1313 or 1314.

7. The system of claim 3, wherein the ZFP domain targets a nucleotide sequence selected from SEQ ID NOs: 700-754.

8. The system of any one of claims 1-7, wherein the DNMT3A domain comprises a sequence with at least 90% identity to SEQ ID NO: 574 or 575.

9. The system of any one of claims 1-8, wherein the DNMT3L domain comprises a sequence with at least 90% identity to a sequence selected from SEQ ID NOs: 578-581.

10. The system of any one of claims 1-8, wherein the DNMT3L domain comprises a sequence with at least 90% identity to a sequence selected from SEQ ID NOs: 582-603.

11. The system of any one of claims 1-7, wherein the DNMT domain comprises a sequence with at least 90% identity to a sequence selected from SEQ ID NOs: 601-603.

12. The system of any one of claims 1-11, wherein the transcriptional repressor domain comprises a sequence with at least 90% identity to a sequence selected from SEQ ID NOs: 33-570.

13. The system of any one of claims 1-11, wherein the transcriptional repressor domain comprises a KRAB domain derived from KOX1, ZIM3, ZFP28, or ZN627.

14. The system of claim 13, wherein the KRAB domain comprises a sequence with at least 90% identity to a sequence selected from SEQ ID NOs: 89, 116, 245, and 255.

15. The system of any one of claims 1-11, wherein the transcriptional repressor domain comprises a fusion of the N- and C-terminal regions of ZIM3 and KOX1 KRAB, and optionally comprises the amino acid sequence of SEQ ID NO: 571 or 572.

16. The system of any one of claims 1-11, wherein the transcriptional repressor domain is derived from KAP1, MECP2, HP1a/CBX5, HP1b, CBX8, CDYL2, TOX, TOX3, TOX4, EED, EZH2, RBBP4, RCOR1, or SCML2.

17. The system of any one of claims 1-16, wherein the system comprises
a) a fusion protein comprising the DNMT3A domain, the DNMT3L domain, the transcriptional repressor domain, and the DNA-binding domain,
optionally wherein one or both of the DNMT3A domain and the DNMT3L domain are human, and
optionally wherein the DNA-binding domain is a dead CRISPR Cas domain or a ZFP domain; or
b) a nucleic acid molecule encoding the fusion protein.

18. The system of claim 17, wherein the fusion protein comprises, from N-terminus to C-terminus, the DNMT3A domain, a first peptide linker, the DNMT3L domain, a second peptide linker, the DNA-binding domain, a third peptide linker, and the transcriptional repressor domain.

19. The system of claim 17, wherein the fusion protein comprises, from N-terminus to C-terminus, the DNMT3A domain, the first peptide linker, the DNMT3L domain, the second peptide linker, a first nuclear localization signal (NLS), the DNA-binding domain, a second NLS, the third peptide linker, and the transcriptional repressor domain.

20. The system of claim 17, wherein the fusion protein comprises, from N-terminus to C-terminus, a first nuclear localization signal (NLS), the DNMT3A domain, the first peptide linker, the DNMT3L domain, the second peptide linker, the DNA-binding domain, the third peptide linker, the transcriptional repressor domain, and a second NLS.

21. The system of claim 17, wherein the fusion protein comprises, from N-terminus to C-terminus, first and second nuclear localization signals (NLSs), the DNMT3A domain, the first peptide linker, the DNMT3L domain, the second peptide linker, the DNA-binding domain, the third peptide linker, the transcriptional repressor domain, and third and fourth NLSs.

22. The system of any one of claims 17-21, wherein the transcriptional repressor domain is a KRAB domain, optionally a human KOX1, ZFP28, ZN627, or ZIM3 KRAB domain.

23. The system of any one of claims 18-22, wherein one or both of the second and third peptide linkers are XTEN linkers, optionally selected from XTEN80 and XTEN16, and further optionally wherein the second peptide linker is XTEN80, and the third peptide linker is XTEN16.

24. The system of claim 17, wherein the fusion protein comprises, from N-terminus to C-terminus, a human DNMT3A domain, a first peptide linker, a human DNMT3L domain, an XTEN80 peptide linker, a first NLS, a dSpCas9 domain, a second NLS, an XTEN16 peptide linker, and a human KOX1 KRAB domain.

25. The system of claim 24, wherein the fusion protein comprises SEQ ID NO: 658 or a sequence at least 90% identical thereto.

26. The system of claim 17, wherein the fusion protein comprises, from N-terminus to C-terminus, a human DNMT3A domain, a first peptide linker, a human DNMT3L domain, an XTEN80 peptide linker, a first NLS, a ZFP domain, a second NLS, an XTEN16 linker, and a human KOX1 KRAB domain.

27. The system of claim 26, wherein the fusion protein comprises SEQ ID NO: 659 or a sequence at least 90% identical thereto.

28. The system of claim 17, wherein the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human KOX1 KRAB domain, and third and fourth NLSs.

29. The system of claim 28, wherein the fusion protein comprises SEQ ID NO: 660 or a sequence at least 90% identical thereto.

30. The system of claim 17, wherein the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human KOX1 KRAB domain, and third and fourth NLSs.

31. The system of claim 17, wherein the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human ZFP28 KRAB domain, and third and fourth NLSs.

32. The system of claim 31, wherein the fusion protein comprises SEQ ID NO: 661 or a sequence at least 90% identical thereto.

33. The system of claim 17, wherein the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human ZFP28 KRAB domain, and third and fourth NLSs.

34. The system of claim 17, wherein the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human ZN627 KRAB domain, and third and fourth NLSs.

35. The system of claim 34, wherein the fusion protein comprises SEQ ID NO: 662 or a sequence at least 90% identical thereto.

36. The system of claim 17, wherein the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human ZN627 KRAB domain, and third and fourth NLSs.

37. The system of claim 17, wherein the fusion protein comprises, from N-terminus to C-terminus, first and second NLSs, a human DNMT3A domain, a first peptide linker, a human DNMT3L domain, an XTEN80 peptide linker, a dSpCas9 domain, an XTEN16 peptide linker, a human ZIM3 KRAB domain, and third and fourth NLSs.

38. The system of claim 37, wherein the fusion protein comprises SEQ ID NO: 663 or a sequence at least 90% identical thereto.

39. The system of claim 17, wherein the fusion protein comprises, from N-terminus to C-terminus, first and second NLSs, a human DNMT3A domain, a first peptide linker, a human DNMT3L domain, an XTEN80 peptide linker, a ZFP domain, an XTEN16 peptide linker, a human ZIM3 KRAB domain, and third and fourth NLSs.

40. The system of any one of claims 19-39, wherein at least one of the NLSs is an SV40 NLS.

41. The system of any one of claims 1 and 3-16, wherein the system comprises:
a) a first fusion protein comprising a first DNA-binding domain and comprising or recruiting the DNMT3A domain,
a second fusion protein comprising a second DNA-binding domain and comprising or recruiting the DNMT3L domain, and
a third fusion protein comprising a third DNA-binding domain and comprising or recruiting the transcriptional repressor domain; or
b) one or more nucleic acid molecules encoding the fusion proteins.

42. A human cell comprising the system of any one of claims 1-41, or progeny of the cell, optionally wherein the cell is a T lymphocyte or a NK cell.

43. A human cell modified by the system of any one of claims 1-41, or progeny of the cell, optionally wherein the cell is a T lymphocyte or a NK cell, optionally wherein the cell was modified ex vivo.

44. A pharmaceutical composition comprising the system of any one of claims 1-41 and a pharmaceutically acceptable excipient, optionally wherein
the composition comprises lipid nanoparticles (LNPs) comprising the system, and/or
the DNA-binding domain is a dCas domain and the LNPs further comprise one or more gRNAs.

45. A pharmaceutical composition comprising human cells of claim 42 or 43 and a pharmaceutically acceptable excipient.

46. A method of treating a patient in need thereof, comprising administering the system of any one of claims 1-41, human cells of claim 42 or 43, or the pharmaceutical composition of claim 44 or 45 to the patient.

47. The method of claim 46, wherein the patient has cancer or autoimmune disease.

48. The system of any one of claims 1-41, human cells of claim 42 or 43, or the pharmaceutical composition of claim 44 or 45, for use in treating a patient in need thereof, optionally in the method of claim 46 or 47.

49. Use of the system of any one of claims 1-41 or the human cells of claim 42 or 43 in the manufacture of a medicament for treating a patient in need thereof, optionally in the method of claim 46 or 47.
